(19) United States
(12) Patent Application Publication
(10) Pub. No.: US 2004/0101823 A1
(43) Pub. Date: May 27, 2004

(54) HIV-1 viral variants for improved animal models of HIV-1 pathogenesis

(75) Inventors: Nay Wei Soong, San Jose, CA (US); Katja Pekrun, East Palo, CA (US); Riri Shibata, San Francisco, CA (US)

Correspondence Address: MAXYGEN, INC., INTELLECTUAL PROPERTY DEPARTMENT, 515 GALVESTON DRIVE, RED WOOD CITY, CA, 94063, US

(73) Assignee: Maxygen, Inc., Redwood City, CA

(21) Appl. No.: 10/325,468

(22) Filed: Dec. 19, 2002

Related U.S. Application Data
(60) Provisional application No. 60/343,524, filed on Dec. 21, 2001.

(51) Int. Cl. C12Q 1/70; C12Q 1/68; C07H 21/02

(52) U.S. Cl. 435/5; 435/6; 536/23.72

(57) ABSTRACT

The invention relates to HIV-1 viral variants and nucleic acids and polypeptides thereof having improved replication properties for development of suitable animal models for the study of HIV-1 pathogenesis, monkey models of HIV-1 infection comprising such variants, and methods for screening for agents that inhibit or control HIV-1 infection.

### CROSS-REFERENCE TO RELATED APPLICATIONS

This application claims the benefit of U.S. Provisional Application No. 60/343,524, filed Dec. 21, 2001, which is incorporated herein by reference in its entirety for all purposes.

### STATEMENT AS TO RIGHTS TO INVENTIONS MADE UNDER FEDERALLY SPONSORED RESEARCH AND DEVELOPMENT

The invention was made with United States Government support under Grant No. 97-01-0240 from the National Institutes of Standards and Technology's Advanced Technology Program. The United States Government has certain rights in the invention.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The invention pertains to variants of viral nucleic acids and polypeptides and virus variants, such as HIV-1 derived virus variants and HIV-1 derived nucleic acid and polypeptide variants, and animal models of viral infection, and the development of therapeutic and prophylactic strategies to viral infection and diseases.

### BACKGROUND OF THE INVENTION

The narrow host range of HIV-1 has impeded development of a suitable animal model for the study of HIV-1 pathogenesis and for testing prophylactic and therapeutic strategies to control virus infections. Although HIV-1 infects chimpanzees, the infected animals rarely develop any AIDS-like symptoms. Furthermore, chimpanzees are endangered, expensive and difficult to handle. Unlike the great apes, macaques are not endangered and are relatively easy to breed. Among the macaques tested, only the pig-tailed macaque (Macaca nemestrina) has been reported to support HIV-1 replication (Agy 1992, Science, 257: 103-106). However, HIV-1 replicates poorly in pig-tailed macaques (Gartner 1994, AIDS Res. Hum. Retroviruses, 10: S129-133; Gartner 1994, J. Med. Primatol., 23: 155-163; Agy 1997, Virology, 238: 336-343), causing only small and transient declines in CD4 cells and no disease (Bosch 1997, J. Acquir. Immune Defic. Syndr., 11: 1555-1563). In contrast, infection of macaques with SIV often results in robust replication and AIDS-like symptoms, making this a more relevant model for HIV-1 induced disease. Its utility though, for testing anti-HIV-1 drugs and vaccines is limited by the significant genetic divergence between SIV and HIV-1 (Chakrabarti 1987, Nature, 328: 543-547; Franchini 1987, Nature, 328: 539-543; Hirsch 1987, Cell, 49: 307-319; Franchini 1989, Ann. NY Acad. Sci., 554: 81-87).

The development of chimeric simian-human immunodeficiency viruses (SHIV) harboring the HIV-1 envelope and regulatory genes (rev, tat and vpu) in an SIV background has only partially solved this problem (Shibata, Kawamura et al. 1991, J. Virol., 65: 3514-3520; Li 1992, J. Acquir. Immune Defic. Syndr., 5: 639-646; Luciw 1995, Proc. Natl. Acad. Sci. USA, 92: 7490-7494; Shibata 1997, J. Infect. Dis., 176: 362-373). These SHIVs are useful for testing HIV-1 envelope based vaccine candidates. Several pathogenic SHIVs with different HIV-1 envelopes are available (Joag 1996, J. Virol., 70: 3189-3197; Reimann 1996, J. Virol., 70: 6922-6928; Igarashi, Endo et al. 1999, PNAS, 96: 14049-14054; Luciw, Mandell et al. 1999, Virology, 263:). Since the 5′ regions of their genomes are composed of SIV sequences, these SHIVs cannot be used for testing cytotoxic T-lymphocyte (CTL) inducing vaccines targeted at Gag.

There exists a need for HIV-1 viral variants having improved replication properties and suitable animal models of HIV-1 infection. The present invention fulfills this and other needs. The present invention provides HIV-1 viral variants having improved replication properties and animal models comprising such variants that are useful for the study of HIV-1 pathogenesis and for testing prophylactic and therapeutic strategies and agents for treating and controlling HIV infection.

### SUMMARY OF THE INVENTION

DNA shuffling facilitated the evolution of human immunodeficiency virus type 1 (HIV-1) variants with enhanced replication in pig-tailed macaque peripheral blood mononuclear cells (pt mPBMC). Such variants are useful for generating suitable animal models for the study of the pathogenesis of HIV-1 infection and for testing prophylactic and therapeutic strategies and agents to control and/or inhibit virus infections.

These virus variants comprise HIV-1 derived sequences with the exception of simian immunodeficiency virus (SIV) nef. Briefly, sequences spanning the gag-protease-reverse transcriptase (gag-pro-RT) region from several HIV-1 isolates were shuffled and cloned into a parental HIV-1 backbone containing SIV nef. Neither this full-length parent nor any of the unshuffled HIV-1 isolates replicated appreciably or sustainably in pt mPBMC. Upon selection of the shuffled viral libraries by serial passaging in pt mPBMC, a species emerged which replicated at substantially higher levels (50-100 ng/ml p24) than any of the HIV-1 parents and most importantly, could be continuously passaged in pt mPBMC. The parental HIV-1 isolates, when selected similarly, became extinct. Analyses of full-length improved proviral clones indicated that multiple recombination events in the shuffled region and adaptive changes in the rest of the genome contributed synergistically to the improved phenotype. These improved variants are useful in establishing a pig-tailed macaque model of HIV-1 infection.

The invention provides novel recombinant or chimeric HIV-1 nucleic acids that encode recombinant or chimeric HIV-1 viruses that exhibit enhanced replication in non-human mammalian cells, including both in vitro and in vivo. The sequences were obtained by performing DNA shuffling of the gag-pro-RT regions using several parental HIV-1 sequences. The resulting recombinant or chimeric HIV-1 sequences were screened in vitro using pig-tailed macaque monkey cells to identify the recombinant or chimeric HIV-1 sequences that encoded HIV virus that exhibited enhanced replication. The invention also provides vectors, HIV-1 viruses, cells, and compositions comprising these recombinant or chimeric HIV-1 nucleic acids.

In one aspect, the invention provides a chimeric or recombinant nucleic acid comprising a polynucleotide sequence that has at least about 80, 85, 88, 90, 91, 92, 93, 94, 95, 96, 97, 98.5, 99, 99.5% or 100% sequence identity to at least one polynucleotide sequence selected from the group of SEQ ID NO:1 to SEQ ID NO:7 or a complementary polynucleotide sequence thereof. In some aspects, such chimeric or recombinant nucleic acid produces a chimeric or recombinant human immunodeficiency virus type 1 (HIV-1) variant that exhibits enhanced replication in macaque monkey cells compared to the replication of an HIV-1 virus, such as a wild-type (WT) HIV-1 virus, in macaque monkey cells. In some aspects, the chimeric or recombinant nucleic acid of claim 2, wherein a first polynucleotide subsequence comprising an HIV-1 nef gene has been replaced with a second polynucleotide subsequence comprising a simian immunodeficiency virus (SIV) nef gene. In other aspects, the polynucleotide sequence comprises a polynucleotide sequence selected from the group of SEQ ID NOS:1-7 or a complementary polynucleotide sequence thereof.

The invention also provides a chimeric or recombinant nucleic acid comprising a polynucleotide sequence selected from the group of: (a) a polynucleotide sequence that encodes nine polypeptides, said nine polypeptides comprising the amino acid sequences of SEQ ID NO:8 to SEQ ID NO:16, or a complementary polynucleotide sequence thereof; (b) a polynucleotide sequence that encodes nine polypeptides, said nine polypeptides comprising the amino acid sequences of SEQ ID NO:17, SEQ ID NO:9, SEQ ID NO:18, SEQ ID NO:11, SEQ ID NO:12, SEQ ID NO:13, SEQ ID NO:14, SEQ ID NO:15, and SEQ ID NO:19, or a complementary polynucleotide sequence thereof; (c) a polynucleotide sequence that encodes nine polypeptides, the nine polypeptides comprising the amino acid sequences of SEQ ID NO:8, SEQ ID NO:20, SEQ ID NO:10, SEQ ID NO:11, SEQ ID NO:12, SEQ ID NO:13, SEQ ID NO:14, SEQ ID NO:14, and SEQ ID NO:21, respectively, or a complementary polynucleotide sequence thereof; (d) a polynucleotide sequence that encodes nine polypeptides, the nine polypeptides comprising the amino acid sequences of SEQ ID NO:22, SEQ ID NO:23, SEQ ID NO:10, SEQ ID NO:11, SEQ ID NO:12, SEQ ID NO:24, SEQ ID NO:14, SEQ ID NO:25, and SEQ ID NO:26, or a complementary polynucleotide sequence thereof, (e) a polynucleotide sequence that encodes nine polypeptides, the nine polypeptides comprising the amino acid sequences of SEQ ID NO:27, SEQ ID NO:9, SEQ ID NO:28, SEQ ID NO:11, SEQ ID NO:12, SEQ ID NO:13, SEQ ID NO:14, SEQ ID NO:29, and SEQ ID NO:30, or a complementary polynucleotide sequence thereof, (f) a polynucleotide sequence that encodes nine polypeptides, the nine polypeptides comprising the amino acid sequences of SEQ ID NO:8, SEQ ID NO:9, SEQ ID NO:31, SEQ ID NO:11, SEQ ID NO:12, SEQ ID NO:13, SEQ ID NO:14, SEQ ID NO:32, and SEQ ID NO:33, or a complementary polynucleotide sequence thereof, and (g) a polynucleotide sequence that encodes nine polypeptides, the nine polypeptides comprising the amino acid sequences of SEQ ID NO:34, SEQ ID NO:35, SEQ ID NO:10, SEQ ID NO:36, SEQ ID NO:12, SEQ ID NO:13, SEQ ID NO:37, SEQ ID NO:38, and SEQ ID NO:39, or a complementary polynucleotide sequence thereof. In some such aspects, the polynucleotide sequence comprises a deoxyribonucleic acid (DNA) polynucleotide sequence.

The invention also provides a chimeric or recombinant nucleic acid comprising a polynucleotide sequence comprising from at least about nucleic acid residue 530 to at least about nucleic acid residue 9859 of a chimeric or recombinant nucleic acid of the invention described above or herein (e.g., SEQ ID NO:1 to SEQ ID NO:7), wherein each thymine residue is replaced by a uracil residue, or a complementary polynucleotide sequence thereof.

In yet another aspect, the invention provides a chimeric or recombinant nucleic acid comprising a polynucleotide sequence of a chimeric or recombinant nucleic acid of the invention described above or herein (e.g., SEQ ID NO:1 to SEQ ID NO:7), wherein each thymine is replaced by a uracil, or a complementary polynucleotide sequence thereof.

Also provided is a chimeric or recombinant RNA polynucleotide sequence comprising from at least about nucleic acid residue 530 to at least about nucleic acid residue 9859 of a polynucleotide sequence selected from the group of SEQ ID NO:1 to SEQ ID NO:7, wherein each thymine is replaced by a uracil, or a complementary polynucleotide sequence thereof.

The invention further provides a chimeric or recombinant nucleic acid comprising a polynucleotide sequence comprising a polynucleotide sequence selected from the group of SEQ ID NO:1 to SEQ ID NO:7, wherein each thymine is replaced by a uracil, or a complementary sequence of said polynucleotide sequence. In another aspect, the invention provides a chimeric or recombinant ribonucleic acid (RNA) comprising an RNA polynucleotide sequence transcribed from at least about nucleic acid residue 530 to at least about nucleic acid residue 9859 of a nucleic acid comprising a polynucleotide sequence selected from the group of SEQ ID NO:1 to SEQ ID NO:7, or a complementary sequence of said RNA polynucleotide sequence.

Other aspects of the invention include isolated, chimeric or recombinant nucleic acid comprising a polynucleotide sequence comprising the gag-pro-reverse transcriptase region of the recombinant or chimeric HIV-1 nucleic acids described above. Also provided is a chimeric or recombinant nucleic acid comprising a polynucleotide sequence comprising from at least about nucleic acid residue 863 to at least about nucleic acid residue 4305 of the chimeric or recombinant nucleic acid of the invention as described above or herein (e.g., SEQ ID NO:1 to SEQ ID NO:7), or a complementary sequence thereof. In one aspect, such chimeric or recombinant nucleic acid comprising a polynucleotide sequence comprising from at least about nucleic acid residue 785 to at least about nucleic acid residue 4305 of the chimeric or recombinant nucleic acid of SEQ ID NO:1 to SEQ ID NO:7. Some such chimeric or recombinant nucleic acids comprise DNA sequences. The invention provides an HIV-1 virus, e.g., DH12, wherein the gag-pro-reverse transcriptase genes are replaced by the corresponding genes from the recombinant or chimeric HIV-1 nucleic acids of the invention. Accordingly, the invention includes a chimeric or recombinant nucleic acid comprising a nucleotide sequence of an HIV-1 virus in which a nucleotide subsequence corresponding to a gag coding region, a pro coding region, and a reverse transcriptase (RT) coding region of the HIV-1 nucleotide sequence is replaced by such chimeric or recombinant nucleic acid. In some such aspects, the HIV-1 virus comprises DH12.

Another aspect of the invention includes isolated, chimeric or recombinant nucleic acid comprising a polynucleotide sequence comprising the gag region of the recombinant or chimeric HIV-1 nucleic acids described above. The invention provides a chimeric or recombinant nucleic acid comprising a polynucleotide sequence comprising from at least about nucleic acid residue 863 to at least about nucleic acid residue 2365 of the chimeric or recombinant nucleic acid of the invention described above or herein (e.g., SEQ ID NOS:1-7). Also provided is a chimeric or recombinant nucleic acid comprising a nucleotide sequence of an HIV-1 virus in which a nucleotide subsequence corresponding to the gag coding region gene of the HIV-1 virus nucleotide sequence is replaced by such chimeric or recombinant nucleic acid. The HIV-1 virus may comprise DH12. The invention further provides an HIV-1 virus, e.g., DH12, wherein the gag gene is replaced by the corresponding gene from the recombinant or chimeric HIV-1 nucleic acids of the invention.

Other aspects of the invention include isolated, chimeric or recombinant nucleic acid comprising a polynucleotide sequence comprising the pro-reverse transcriptase region of the recombinant or chimeric HIV-1 nucleic acids described above. Accordingly, the invention includes a chimeric or recombinant nucleic acid comprising a polynucleotide sequence comprising from at least about nucleic acid residue 2158 to at least about nucleic acid residue 4305 of the chimeric or recombinant nucleic acid of the invention as described above or herein (e.g., SEQ ID NOS:1-7). Some such chimeric or recombinant nucleic acids comprise DNA sequences. In some such aspects, a chimeric or recombinant nucleic acid comprising an HIV-1 virus polynucleotide sequence in which the nucleotide subsequence corresponding to a protease and reverse transcriptase coding regions of the HIV-1 virus nucleotide sequence is replaced by such chimeric or recombinant nucleic acid. The HIV-1 virus comprises DH12. The invention further provides an HIV-1 virus, e.g., DH12, wherein the pro-reverse transcriptase genes are replaced by the corresponding genes from the recombinant or chimeric HIV-1 nucleic acids of the invention.

The invention also includes modified, recombinant, or chimeric HIV-1 viruses. Accordingly, the invention provides a modified, recombinant, or chimeric HIV-1 viruses comprising at least one recombinant or chimeric HIV-1 nucleic acid of the invention. The invention includes a modifed, recombinant, or chimeric HIV-1 virus produced by expression or translation of a recombinant or chimeric HIV-1 nucleic acid of the invention in a population of primate cells. In another aspect, the invention provides a modified or chimeric HIV-1 virus comprising at least one chimeric or recombinant nucleic acid of the invention described above or herein. Also provided is a modified or chimeric HIV-1 virus produced by expression or translation of at least one chimeric or recombinant nucleic acid of the invention as described above or herein in a population of primate cells.

In another aspect, the invention provides a modified or chimeric HIV-1 virus produced by expression or translation of an RNA nucleic acid in a population of primate cells, said RNA nucleic acid comprising from at least about nucleic acid residue 530 to at least about nucleic acid residue 9859 of a chimeric or recombinant nucleic acid of the invention described above or herein, wherein each thymine is replaced by a uracil, or a complementary sequence thereof.

Also provided is a modified or chimeric HIV-1 virus comprising an RNA polynucleotide sequence, said RNA polynucleotide sequence comprising from at least about nucleic acid residue 530 to at least about nucleic acid residue 9859 of a chimeric or recombinant nucleic acid of the invention as described herein (e.g., SEQ ID NOS:1-7), wherein each thymine is replaced by a uracil, or a complementary sequence thereof.

In yet another aspect, the invention provides a modified or chimeric HIV-1 virus comprising an RNA polynucleotide sequence, said RNA polynucleotide sequence comprising from at least about nucleic acid residue 530 to at least about nucleic acid residue 9859 of a chimeric or recombinant nucleic acid of SEQ ID NO:1 to SEQ ID NO:7, wherein each thymine is replaced by a uracil, or a complementary sequence thereof. For some such modified or chimeric HIV-1 virus, the virus replicates in macaque monkey cells in vivo or in vitro. The macaque monkey cells may comprise pig-tailed macaque monkey cells. For some such modified or chimeric HIV-1 virus, the modified or chimeric HIV-1 virus exhibits enhanced replication in a population of macaque monkey cells compared to replication of an HIV-1 virus, such as a WT HIV-1 virus, in a population of macaque monkey cells. In some instances, the modified or chimeric HIV-1 virus grows to a higher titer in a population of macaque monkey cells than does the HIV-1 virus in a population of macaque monkey cells. In some instances, the modified or chimeric HIV-1 virus replicates for a longer period of time in a population of macaque monkey cells than does the HIV-1 virus in a population of macaque monkey cells. In some aspects, the modified or chimeric HIV-1 virus grows at a faster rate in a population of macaque monkey cells than does the HIV-1 virus in a population of macaque monkey cells. In some aspects, the modified or chimeric HIV-1 virus exhibits replication in vivo in a macaque monkey, such as a pig-tailed macaque monkey. In some aspects, the modified or chimeric HIV-1 virus exhibits enhanced replication in vivo in the pig-tailed macaque monkey compared to replication of an HIV-1 virus, such as a WT HIV-1 virus in vivo in said pig-tailed macaque monkey.

The invention further provides a non-human primate comprising or infected with one or more modified or chimeric HIV-1 viruses of the invention described above or herein. In some aspects, the non-human primate is a macaque monkey, preferably a pig-tailed macaque monkey.

Also provided is a non-human primate comprising at least one nucleic acid of the invention as described above or herein. The non-human primate may be a macaque monkey, such as a pig-tailed macaque monkey. In some aspects, the modified or chimeric HIV-1 virus replicates for a longer period of time in the macaque monkey than does an HIV-1 virus in the macaque monkey. In some aspects, the macaque monkey comprising the modified or chimeric HIV-1 virus exhibits a decrease in a population of CD4+ T cells. In some aspects, the macaque monkey comprising the modified or chimeric HIV-1 virus exhibits an increase in viremia. In some aspects, the macaque monkey comprising at least one modified or chimeric HIV-1 virus exhibits at least one symptom of HIV-1 infection that is sustained for a longer period of time than the period of time the symptom of HIV-1 infection lasts in a macaque monkey comprising an HIV-1 virus. The macaque monkey comprising at least one modified, or chimeric HIV-1 virus may exhibit at least one symptom of HIV infection. In some aspects, the macaque monkey comprising the modified or chimeric HIV-1 virus exhibits at least one symptom associated with acquired immunodeficiency disease syndrome (AIDS).

The invention also provides a method for producing a non-human mammalian cell comprising a modified or chimeric HIV-1 virus that exhibits enhanced replication in a non-human mammalian cell compared to replication of an HIV-1 virus, such as a WT HIV-1 virus, in said non-human mammalian cell, said method comprising administering to the non-human mammalian cell in vitro or in vivo a modified or chimeric HIV-1 virus comprising the nucleic acid of the invention as described above or herein (e.g., SEQ ID NOS:1-7). Also provided is a non-human mammalian cell produced by such method.

In another aspect, the invention provides a method for producing a non-human mammalian cell comprising a modified or chimeric HIV-1 virus that exhibits enhanced replication in a non-human mammalian cell compared to replication of an HIV-1 virus, such as a WT HIV-1 virus strain, in said non-human mammalian cell, said method comprising administering to the non-human mammalian cell the modified or chimeric HIV-1 virus of the invention as described above or herein.

Also included is a method for producing a macaque monkey comprising a modified or chimeric HIV-1 virus that exhibits enhanced replication in a macaque monkey cell compared to replication of an HIV-1 virus, such as a wild-type HIV-1 virus, in said macaque monkey cell, said method comprising administering to a population of cells of the macaque monkey at least one modified or chimeric HIV-1 virus comprising the nucleic acid of the invention in an amount sufficient to cause an HIV infection in said population of cells of the macaque monkey. In some such methods, at least one symptom of HIV infection is produced in the macaque monkey or in a population of cells of the macaque monkey. Also provided is a macaque monkey produced by such method.

In another aspect, the invention provides a method for producing a macaque monkey comprising or infected with at least one modified or chimeric HIV-1 virus that exhibits enhanced replication in a macaque monkey cell compared to replication of an HIV-1 virus, such as a WT HIV-1 virus, in said macaque monkey cell, said method comprising administering to a population of cells of the macaque monkey at least one such modified or chimeric HIV-1 virus of the invention describe above or herein in an amount sufficient to cause an HIV infection in said population of cells of the macaque monkey.

In another aspect, the invention provides chimeric or recombinant polypeptides encoded by each of the chimeric or recombinant nucleic acids of the invention described above or herein. The invention further provides chimeric or recombinant polypeptides comprising an amino acid sequence selected from the group of: (a) an amino acid sequence that has at least about 80, 85, 88, 90, 91, 92, 93, 94, 95, 96, 97, 98.5, 99, 99.5%, or 100% sequence identity to at least one sequence from the group of SEQ ID NOS:8, 17, 22, 27, and 34; (b) an amino acid sequence that has at least about 80, 85, 88, 90, 91, 92, 93, 94, 95, 96, 97, 98.5, 99, 99.5%, or 100% sequence identity to at least one amino acid sequence of the group of SEQ ID NOS:9, 20, 23, and 35; (c)) an amino acid sequence that has at least about 80, 85, 88, 90, 91, 92, 93, 94, 95, 96, 97, 98.5, 99, 99.5%, or 100% sequence identity to at least one amino acid sequence of the group of SEQ ID NOS:10, 18, 28, and 31; (d)) an amino acid sequence that has at least about 80, 85, 88, 90, 91, 92, 93, 94, 95, 96, 97, 98.5, 99, 99.5%, or 100% sequence identity to at least one amino acid sequence of the group of SEQ ID NOS:11 and 36; (e) an amino acid sequence that has at least about 80, 85, 88, 90, 91, 92, 93, 94, 95, 96, 97, 98.5, 99, 99.5%, or 100% sequence identity to SEQ ID NO:12; (f) an amino acid sequence that has at least about 80, 85, 88, 90, 91, 92, 93, 94, 95, 96, 97, 98.5, 99, 99.5%, or 100% sequence identity to at least one amino acid sequence of the group of SEQ ID NOS:13 and 24; (g) an amino acid sequence that has at least about 80, 85, 88, 90, 91, 92, 93, 94, 95, 96, 97, 98.5, 99, 99.5%, or 100% sequence identity to at least one amino acid sequence of the group of SEQ ID NOS:14 and 37; (h) an amino acid sequence that has at least about 80, 85, 88, 90, 91, 92, 93, 94, 95, 96, 97, 98.5, 99, 99.5%, or 100% sequence identity to at least one amino acid sequence of the group of SEQ ID NOS:15, 25, 29, 32, and 38; and (i) an amino acid sequence that has at least about 80, 85, 88, 90, 91, 92, 93, 94, 95, 96, 97, 98.5, 99, 99.5%, or 100% sequence identity to at least one amino acid sequence of the group of SEQ ID NOS:16, 19, 21, 26, 30, 33, and 39. Also included are nucleic acids, including DNA and RNA polynucleotides, that encode one or more such chimeric or recombinant polypeptides. Also included are compositions comprising one or more such chimeric or recombinant polypeptides and an excipient, including a pharmaceutically acceptable excipient.

The invention further includes a cell-culture derived progeny of the modified or chimeric HIV-1 viruses of the invention described above or herein. Some such progeny exhibit replication in a macaque monkey and the macaque monkey comprising the progeny exhibits at least one symptom of HIV infection.

The invention further provides an evolved virus produced by passaging a viral isolate at least one time through macaque monkey cells, tissue, or blood, wherein the viral isolate comprises at least one modified or chimeric virus of the invention described above or herein. In some instances, the macaque monkey comprising said evolved virus develops at least one symptom of HIV infection. The evolved virus may exhibit enhanced replication in macaque monkey cells, tissue, or blood compared to replication in macaque monkey cells, tissue, or blood of the modified or chimeric virus prior to a first passage.

Also provided is a method for producing an evolved HIV-1 virus that replicates and causes at least one symptom of HIV infection in a macaque monkey, the method comprising passaging a viral isolate comprising a virus in vivo by one or more successive passages through macaque monkey cells, tissue, or blood, wherein prior to the first passage the virus comprised at least one modified or chimeric virus of the invention (e.g., SEQ ID NOS:1-7). In another aspect, the invention provides a method for producing a macaque monkey exhibiting at least one symptom of HIV infection, said method comprising administering to the macaque monkey an evolved viral isolate comprising a virus that has been passaged in vivo at least one time through macaque monkey cells, tissue or blood prior to administration to the macaque monkey, wherein prior to the first passage the comprised at least one such modified or chimeric nucleic acid of the invention (e.g., SEQ ID NOS:1-7).

Other aspects of the invention relate to a method of producing a further modified or recombinant nucleic acid that entails mutating or recombining the recombinant or chimeric HIV-1 nucleic acid of the invention. In one embodiment, the method entails recursively recombining the recombinant or chimeric HIV-1 nucleic acid of the invention with one or more additional nucleic acids. In alternate embodiments, the mutating or recombining is performed in vitro or in vivo. In a further embodiment, the method entails producing at least one library of further modified or recombinant nucleic acids, said library comprising at least one nucleic acid, wherein at least one modified or chimeric HIV-1 virus comprising said at least one nucleic acid exhibits enhanced replication in non-human mammalian cells. The invention also includes the library produced by this method and a population of cells comprising this library.

The invention also includes the further modified or recombinant nucleic acid produced by this method, wherein at least one modified or chimeric HIV-1 virus comprising said further modified or recombinant nucleic acid exhibits enhanced replication in non-human mammalian cells. In one embodiment, the enhanced replication comprises an ability to replicate at a greater rate or for a longer period in vitro in macaque monkey cells or in vivo in a macaque monkey compared to an HIV-1 virus ability to replicate in vitro in macaque monkey cells or in vivo in a macaque monkey.

The invention further provides a method of screening for an agent that inhibits and/or treats HIV infection. In some aspects, the invention provides a method of screening for an agent that inhibits and/or treats HIV infection in a non-human primate, said method comprising: (a) administering a test agent to a first non-human primate; (b) administering a modified or chimeric HIV-1 virus comprising the nucleic acid of the invention described above or herein to the first non-human primate in an amount sufficient to cause HIV infection; (c) administering the modified or chimeric HIV-1 virus comprising the nucleic acid of the invention described above or herein to a second non-human primate in said amount; (d) monitoring a level of HIV infection and/or an appearance of at least one AIDS-associated symptom in each of the first non-human primate and the second non-human primate, wherein a decrease in the level of HIV infection and/or a delay or absence of the appearance of at least one AIDS-associated symptom in the first non-human primate as compared to the level of HIV infection and/or the appearance of at least one AIDS-associated symptom in the second non-human primate indicates that the test agent inhibits HIV infection. In some such methods, the first and second non-human primates are macaque monkeys. In some such methods, the first and second non-human primates are pig-tailed macaque monkeys.

Also provided is a method for screening for an agent that treats HIV infection said method comprising: (a) providing a first macaque monkey and a second macaque monkey, each of which comprises the macaque monkey produced by the method of claim 66; (b) administering a test agent to the first macaque monkey; (c) monitoring a level of HIV infection and/or an appearance of at least one AIDS-associated symptom in each of the first and second macaque monkeys, wherein a decrease in the level of HIV infection and/or a delay or absence of the appearance of at least one AIDS-associated symptom in the first macaque monkey as compared to the level of HIV infection and/or the appearance of at least one AIDS-associated symptom in the second macaque monkey indicates that the test agent treats HIV infection.

In another aspect, the invention provides a method of screening for an agent that inhibits HIV infection, said method comprising: (a) administering a test agent to a first population of primate cells; (b) administering at least one modified or chimeric HIV-1 virus comprising the nucleic acid of the invention (e.g., SEQ ID NOS:1-7) to the first population of primate cells in an amount sufficient to cause HIV infection; (c) administering the at least one modified or chimeric HIV-1 virus comprising the nucleic acid of the invention described herein to a second population of primate cells in the amount; (d) monitoring a level of HIV infection in each of the first population of primate cells and the second population of primate cells, wherein a decrease in the level of HIV infection in the first population of primate cells as compared to the level of HIV infection in the second population of primate cells indicates that the test agent inhibits HIV infection. In some such methods, the primate cells are human cells. In some such methods, the primate cells are macaque monkey cells, such as pig-tailed macaque monkey cells. Some such methods are performed in vitro or ex vivo.

In another aspect, the invention provides a method for screening for an agent that treats HIV infection, said method comprising: (a) providing a first population of macaque monkey cells and a second population of macaque monkey cells, said first and second population of cells comprising one or more cells of the invention described above or one or more cells comprising the cell-culture derived progeny of the invention described above; (b) administering a test agent to the first population of macaque monkey cells; (c) monitoring a level of HIV infection in the first and second populations of macaque monkey cells, wherein a decrease in the level of HIV infection in the first population of macaque monkey cells as compared to the level of HIV infection in the second population of macaque monkey cells indicates that the test agent inhibits HIV infection. In some such methods, the first and second populations of macaque monkey cells are pig-tailed macaque monkey cells.

The invention further provides a chimeric nucleic acid that encodes a chimeric or modified HIV-1 virus that exhibits replication in macaque monkey cells, wherein said nucleic acid comprises a polynucleotide sequence of an HIV-1 virus in which a first nucleotide subsequence of the HIV-1 polynucleotide sequence, said nucleotide subsequence comprising a gag coding sequence, a pro coding sequence, and a reverse transcriptase sequence, is substituted with a first chimeric nucleotide subsequence of the nucleic acid sequence of any of SEQ ID NOS:1-7, said first chimeric nucleotide subsequence comprising a chimeric gag coding sequence, chimeric a pro coding sequence, and chimeric reverse transcriptase sequence. For some such chimeric nucleic acids, the first chimeric nucleotide subsequence comprises from at least about nucleic acid residue 785 to at least about nucleic acid residue 4305 of a nucleic acid sequence selected from the group of SEQ ID NOS:1-7. For some such chimeric nucleic acids, the first chimeric nucleotide subsequence comprises from at least about nucleic acid residue 785 to at least about nucleic acid residue 4305 of the nucleic acid sequence of SEQ ID NO:1. The HIV-1 virus may comprise DH12.

For some such chimeric nucleic acids, a second nucleotide subsequence of the HIV-1 polynucleotide sequence corresponding to an HIV-1 nef coding region is substituted with a nucleotide subsequence corresponding to a modified simian immunodeficiency virus (SIV) nef region or gene, said modified SIV nef region or gene comprising a SIV nef polynucleotide sequence in which a first codon encoding an arginine (R) amino acid residue at position 17 of the SIV nef amino acid sequence is substituted with a second codon encoding a tyrosine (Y) amino acid residue and a third codon encoding a glutamine (Q) amino acid residue at position 18 of the SIV nef amino acid sequence has been replaced with a fourth codon encoding a glutamic acid (E) amino acid residue. The modified or chimeric virus may exhibit enhanced replication in macaque monkey cells, including pig-tailed macaque monkey cells. In some such chimeric or recombinant nucleic acids, each thymine is substituted with a uracil. The invention also includes a polypeptide encoded by such chimeric or recombinant nucleic acid and a modified or chimeric HIV-1 virus comprising such chimeric nucleic acid.

In preferred embodiments, the modified or chimeric HIV-1 virus encoded by at least one chimeric or recombinant nucleic acid of the invention exhibits enhanced replication in macaque monkey cells, most preferably pig-tailed macaque monkey cells. The invention also provides polypeptides encoded by one or more such chimeric or recombinant nucleic acids of the invention, and a recombinant or chimeric HIV-1 virus comprising at least one chimeric or recombinant nucleic acid of the invention.

The invention provides a chimeric or recombinant nucleic acid that encodes a modified or chimeric HIV-1 virus that has the ability to replicate in macaque monkey cells, the nucleic acid comprising an HIV-1 (e.g., a wild-type HIV-1, e.g., DH-1) nucleic acid having the gag, pro, and reverse transcriptase genes replaced with the corresponding genes from a recombinant or chimeric HIV-1 nucleic acid of the invention. In one embodiment, the recombinant or chimeric HIV-1 nucleic acid of the invention includes a polynucleotide sequence selected from the group of SEQ ID NOS:1-7. In another embodiment, the corresponding genes comprise from at least about nucleic acid residue 785 to at least about nucleic acid residue 4305 of a polynucleotide sequence selected from the group of SEQ ID NOS:1-7. In a still further embodiment, the corresponding genes comprise from at least about nucleic acid residue 785 to at least about nucleic acid residue 4305 of SEQ ID NO:1.

The invention also provides a chimeric or recombinant nucleic acid that encodes at least one modified or chimeric HIV-1 virus that can replicate in macaque monkey cells, the nucleic acid comprising an HIV-1 (e.g., a wild-type HIV-1, e.g., DH-1) nucleic acid having the int vif, vpr, rev, tat, vpu, and env genes replaced with the corresponding genes from a recombinant or chimeric HIV-1 nucleic acid of the invention. The invention includes a chimeric or recombinant nucleic acid that encodes a modified or chimeric HIV-1 virus that exhibits replication in macaque monkey cells, said nucleic acid comprising a polynucleotide sequence of an HIV-1 virus in which a nucleotide subsequence of the HIV-1 virus polynucleotide sequence, said nucleotide subsequence comprising the int, vif, vpr, rev, tat, vpu, and env coding regions is substituted with a chimeric or recombinant nucleotide subsequence comprising a chimeric or recombinant int, vif, vpr, rev, tat, vpu, and env coding regions of a nucleic acid of the invention, including, e.g., a nucleic acid sequence selected from the group of SEQ ID NOS:1-7. In some aspects, the chimeric or recombinant nucleotide subsequence comprises the chimeric or recombinant int, vif, vpr, rev, tat, vpu, and env coding regions comprises from at least about nucleic acid residue 4306 to at least about nucleic acid residue 8547 of a nucleic acid sequence selected from the group of SEQ ID NOS:1-7. In some aspects, the chimeric or recombinant nucleotide subsequence comprising the chimeric or recombinant int, vif, vpr, rev, tat, vpu, and env coding regions comprises from at least about nucleic acid residue 4306 to at least about nucleic acid residue 8547 of SEQ ID NO:1. For some such chimeric or recombinant nucleic acids, the HIV-1 virus comprises DH12. In some instances, a nucleotide subsequence corresponding to an HIV-1 nef coding region is substituted with a nucleotide subsequence corresponding to a modified SIV nef coding region, said modified SIV nef coding region comprising a SIV nef polynucleotide sequence in which a codon encoding an arginine (R) amino acid residue at position 17 of the encoded SIV nef amino acid sequence is substituted with a codon encoding a tyrosine (Y) amino acid residue and a codon encoding a glutamine (Q) amino acid residue at position 18 of the encoded SIV nef amino acid sequence is substituted with a codon encoding a glutamic acid (E) amino acid residue. The modified or chimeric HIV-1 virus may exhibit enhanced replication in macaque monkey cells, such as pig-tailed macaque monkey cells, in vivo. In some such chimeric or recombinant nucleic acids, wherein each thymine is substituted with a uracil. Also provided is a polypeptide encoded by each such chimeric or recombinant nucleic acid and a modified or chimeric HIV-1 virus comprising at least one such chimeric or recombinant nucleic acid.

In another aspect, the invention provides a chimeric or recombinant nucleic acid that encodes a recombinant or chimeric HIV-1 that exhibits replication in macaque monkey cells, said nucleic acid comprising a polynucleotide sequence of an HIV-1 virus in which a nucleotide subsequence of the polynucleotide sequence that comprises a nef-LTR (long-term repeat) coding region is substituted with a chimeric or recombinant nucleotide subsequence comprising a chimeric or recombinant nef-LTR coding region of a nucleic acid selected from the group of SEQ ID NOS:1-7. In some aspects, the chimeric or recombinant nucleotide subsequence comprising the chimeric or recombinant nef-LTR coding region comprises from at least about nucleic acid residue 8548 to at least about nucleic acid residue 9942 of a polynucleotide sequence selected from the group of SEQ ID NOS:1-7. In some aspects, the chimeric or recombinant nucleotide subsequence comprising the chimeric or recombinant nef-LTR coding region comprises from at least about nucleic acid residue 8548 to at least about nucleic acid residue 9942 of SEQ ID NO:1. The HIV-1 virus may comprise a DH12 virus. In some such aspects, a nucleotide subsequence corresponding to an HIV-1 nef coding region is substituted with a nucleotide sequence corresponding to a modified SIV nef coding region, said modified SIV nef coding region comprising a SW nef nucleotide sequence in which a codon encoding an arginine (R) amino acid residue at position 17 of the SIV nef amino acid sequence is substituted with a codon encoding a tyrosine (Y) amino acid residue, and a codon encoding a glutamine (Q) amino acid residue at position 18 of the SIV nef amino acid sequence is substituted with a codon encoding a glutamic acid (E) amino acid residue. In some such aspects, the encoded modified or chimeric HIV-1 virus exhibits enhanced replication in macaque monkey cells (e.g., pig-tailed macaques) in vivo. In some such chimeric or recombinant nucleic acids, each thymine is replaced by a uracil in the polynucleotide sequence. Also provided are polypeptides encoded by such chimeric or recombinant nucleic acids and modified or chimeric HIV-1 viruses comprising at least one such chimeric or recombinant nucleic acid.

Also included in the invention are chimeric or recombinant nucleic acids that encode a modified HIV-1 virus that can replicate in macaque monkey cells, the nucleic acid comprising a recombinant or chimeric HIV-1 nucleic acid of the invention having various genes replaced with corresponding genes from an HIV-1 (e.g., a wild-type HIV-1, e.g., DH-1) virus.

In one embodiment, the HIV-1 nef gene of the chimeric or recombinant nucleic acids of the invention is replaced with a modified SIV nef gene that comprises the amino acid residue changes R17Y and Q18E.

In alternate embodiments, these chimeric or recombinant nucleic acids are RNA or DNA nucleic acids. In one embodiment, these chimeric or recombinant-nucleic acids are RNA, and each thymine residue is replaced by a uracil residue in the recombinant or chimeric HIV-1 nucleic acid. In preferred embodiments, the recombinant or chimeric HIV-1 nucleic acid comprises from about nucleic acid residue 530 to about nucleic acid residue 9859.

In one embodiment, the modified HIV-1 virus encoded by the chimeric or recombinant nucleic acid exhibits enhanced replication in macaque monkey cells in vivo. In preferred embodiments, the macaque monkey cells are pig-tailed macaque monkey cells.

In yet another aspect, the invention provides a chimeric or recombinant nucleic acid that encodes at least one modified or chimeric HIV-1 virus that can replicate in macaque monkey cells, the nucleic acid comprising a recombinant or chimeric HIV-1 nucleic acid of the invention having the gag, pro, and reverse transcriptase genes replaced with the corresponding genes from an HIV-1 (e.g., a wild-type HIV-1, e.g., DH-1) virus. In one embodiment, the invention includes a chimeric or recombinant nucleic acid that encodes a modified or chimeric HIV-1 virus that exhibits replication in macaque monkey cells, said nucleic acid comprising an RNA genome corresponding to a polynucleotide sequence selected from the group of SEQ ID NOS:1-7 in which each thymine residue is replaced by a uracil residue, wherein a first nucleotide subsequence of the first RNA genome comprising a first gag gene, a first protease gene, and a first reverse transcriptase (RT) gene is replaced by a second nucleotide subsequence comprising a second gag gene, a second protease gene, and a second reverse transcriptase gene of a second RNA genome of an HIV-1 virus. In another embodiment, the first nucleotide subsequence comprising the first gag gene, the first protease gene, and the first reverse transcriptase gene of the first RNA genome comprises from at least about nucleic acid residue 863 to at least about nucleic acid residue 4305 of the polynucleotide sequence selected from the group of SEQ ID NOS:1-7 wherein each thymine is replaced by a uracil. In a particular embodiment, the first nucleotide subsequence comprising the first gag gene, the first protease gene, and the first reverse transcriptase gene of the first RNA genome comprises from at least about nucleic acid residue 785 to at least about nucleic acid residue 4305 of the polynucleotide sequence selected from the group of SEQ ID NOS:1-7 wherein each thymine residue is replaced by a uracil residue. In some aspects, the HIV-1 virus comprises DH12. The HIV-1 virus may comprise DH12 and the second nucleotide subsequence comprising the second gag gene, the second pro gene, and the second reverse transcriptase gene of the second RNA genome of DH12 comprises from at least about nucleic acid residue 710 to at least about nucleic acid residue 4223 of the DH12 polynucleotide sequence. In another embodiment, the recombinant or chimeric HIV-1 nucleic acid comprises a DNA polynucleotide sequence corresponding to nucleic acid residues 530-9859 of a polynucleotide sequence selected from the group of SEQ ID NOS:1-7.

The invention also provides a chimeric or recombinant nucleic acid that encodes a modified or chimeric HIV-1 virus that can replicate in macaque monkey cells, the nucleic acid comprising a recombinant or chimeric HIV-1 nucleic acid of the invention having the nef-LTR gene replaced with the corresponding gene from an HIV-1 (e.g., a wild-type HIV-1, e.g., DH-1) virus. In one embodiment, the recombinant or chimeric nucleic acid comprises a recombinant DNA polynucleotide sequence corresponding to nucleic acid residues 530 to 9859 of a first polynucleotide sequence selected from the group of SEQ ID NOS:1-7, wherein a first nucleotide subsequence of the first DNA polynucleotide sequence comprising a first gag gene, a first protease gene, and a first reverse transcriptase gene is replaced by a second nucleotide subsequence comprising a second gag gene, a second protease gene, and a second reverse transcriptase gene of an HIV-1 virus DNA polynucleotide sequence. For some such chimeric or recombinant nucleic acids, the resulting modified or chimeric HIV-1 virus exhibits enhanced replication in macaque monkey cells in vivo or in vitro. Also included are polypeptides encoded by at least one such chimeric or recombinant nucleic acid and modified or chimeric HIV-1 viruses comprising at least one such chimeric or recombinant nucleic acid.

In another aspect, the invention provides a chimeric or recombinant nucleic acid that encodes a modified or chimeric HIV-1 virus variant that exhibits replication in macaque monkey cells, said nucleic acid comprising an RNA polynucleotide sequence comprising from about nucleic acid residue 530 to about nucleic acid residue 9859 of a polynucleotide sequence selected from the group of SEQ ID NOS:1-7 in which each thymine is substituted with a uracil, wherein a first nucleotide subsequence of said RNA sequence comprising first integrase (int), vif, vpr, rev, tat, vpu, and envelope (env) coding regions or genes of said RNA sequence is replaced by a second nucleotide subsequence comprising second int, vif, vpr, rev, tat, vpu, and env genes of an HIV-1 virus polynucleotide sequence. In some such chimeric or recombinant nucleic acids, the first RNA sequence comprises from at least about nucleic acid residue 4306 to at least about nucleic acid residue 8547 of a polynucleotide sequence from the group of SEQ ID NOS:1-7 in which each thymine is substituted with a uracil. The HIV-1 virus may comprise DH12. In some such aspects, the HIV-1 virus comprises DH12 and the second polynucleotide subsequence comprising the int, vif, vpr, rev, tat, vpu, and envelope genes of the DH12 polynucleotide sequence comprises from at least about nucleic acid residue 4224 to at least about nucleic acid residue 8465 of the DH12 polynucleotide sequence, wherein each thymine is replaced by a uracil.

In other aspects the invention also includes a chimeric or recombinant nucleic acid that encodes a modified or chimeric HIV-1 virus variant that exhibits replication in macaque monkey cells, said nucleic acid comprising a DNA polynucleotide sequence comprising from about nucleic acid residue 530 to about nucleic acid residue 9859 of a polynucleotide sequence selected from the group of SEQ ID NOS:1-7. In some such aspects, a first polynucleotide subsequence of said DNA polynucleotide sequence comprising first int, vif, vpr, rev, tat, vpu, and env genes is replaced by a second polynucleotide subsequence comprising second int, vif, vpr, rev, tat, vpu, and env genes of an HIV-1 virus polynucleotide sequence. In some such aspects, the modified or chimeric HIV-1 virus exhibits enhanced replication in macaque monkey cells, such as pig-tailed macaque monkey cells, in vivo or in vitro. Also included are polypeptides encoded by at least one such chimeric or recombinant nucleic acid and modified or chimeric HIV-1 virus comprising at least one such chimeric or recombinant nucleic acid of claim 141 or 145.

In other aspects, the invention provides a chimeric or recombinant nucleic acid that encodes a modified or chimeric HIV-1 virus variant that exhibits replication in macaque monkey cells, said nucleic acid comprising a RNA sequence comprising from about nucleic acid residue 530 to about nucleic acid residue 9859 of a polynucleotide selected from the group of SEQ ID NOS:1-7 wherein each thymine is replaced by a uracil, and wherein a first polynucleotide subsequence of said RNA sequence comprising a first nef-LTR gene is replaced by a second polynucleotide subsequence comprising a second nef-LTR gene of an HIV-1 virus polynucleotide sequence. In some such aspects, the first nef-LTR gene of the RNA sequence comprises from at least about nucleic acid residue 8548 to at least about nucleic acid residue 9942 of the polynucleotide sequence from the group of SEQ ID NOS:1-7, wherein each thymine is replaced by a uracil. In some such aspects, the first nef-LTR gene of the RNA sequence comprises from at least about nucleic acid residue 8548 to at least about nucleic acid residue 9942 of SEQ ID NO:1, wherein each thymine is replaced by a uracil. In some such aspects, the HIV-1 virus comprises DH12. In some aspects, the HIV-1 virus comprises DH12 and the second polynucleotide subsequence comprising the second nef-LTR gene of the DH12 polynucleotide sequence comprises from at least about nucleic acid residue 8466 to at least about nucleic acid residue 9704 of the DH12 polynucleotide sequence. The invention further provides polypeptides encoded by such chimeric or recombinant nucleic acids and modified or chimeric HIV-1 viruses comprising at least one such chimeric or recombinant nucleic acid.

In another aspect, the invention also provides a chimeric or recombinant nucleic acid that encodes a modified or chimeric virus that exhibits replication in macaque monkey cells, said nucleic acid comprising a DNA polynucleotide sequence comprising from about nucleic acid residue 530 to about nucleic acid residue 9859 of a polynucleotide sequence selected from the group of SEQ ID NOS:1-7, wherein a first polynucleotide subsequence of said DNA sequence comprising a first nef-LTR gene is replaced by a second polynucleotide subsequence comprising a second nef-LTR gene of an HIV-1 virus polynucleotide sequence.

For some such above-described chimeric or recombinant nucleic acids, a third polynucleotide subsequence corresponding to an HIV-1 nef gene is replaced with a fourth polynucleotide subsequence corresponding to a modified SIV nef gene, said modified SIV nef gene comprising a SIV nef polynucleotide sequence in which a first codon encoding an arginine (R) amino acid residue at position 17 of the SIV nef amino acid sequence is replaced with a second codon encoding a tyrosine (Y) amino acid residue and a third codon encoding a glutamine (Q) amino acid residue at position 18 of the SIV nef amino acid sequence has been replaced with a fourth codon encoding a glutamic acid (E) amino acid residue. For some such above-described chimeric or recombinant nucleic acids, the modified or chimeric HIV-1 virus exhibits enhanced replication in macaque monkey cells (e.g., pig-tailed macaque cells) in vivo. The invention further provides polypeptides encoded by such chimeric or recombinant nucleic acids and modified or chimeric HIV-1 viruses comprising at least one such chimeric or recombinant nucleic acid.

The invention also provides a chimeric or recombinant nucleic acid comprising a polynucleotide sequence that, but for the degeneracy of the genetic code, hybridizes under stringent conditions over substantially the entire length of any chimeric or recombinant nucleic acid of the invention as described above or herein. The invention also includes a chimeric or recombinant nucleic acid comprising a polynucleotide sequence that, but for the degeneracy of the genetic code, hybridizes under stringent conditions over substantially the entire length of a nucleic acid comprising a polynucleotide sequence selected from the group of SEQ ID NOS:1-7, or a complementary polynucleotide sequence thereof.

Also provided are vectors comprising at least one chimeric or recombinant nucleic acid of the invention as described above or herein. The vector may comprise a plasmid, a cosmid, or a phage, or encodes a virus or virus-like particle (VLP). In some instances, the vector comprises an expression vector. Also included are cells comprising one or more such vectors and cells comprising at least one chimeric or recombinant nucleic acid of the invention as set forth herein. In some aspects, the cell expresses at least one polypeptide encoded by the at least one nucleic acid of the invention. Compositions comprising at least one nucleic acid of the invention and an excipient are also provided. The excipient may comprise a pharmaceutically acceptable excipient.

The invention also provides one or more cells comprising a modified or chimeric HIV-1 virus of the invention as described above or herein. The cell may comprises a mammalian cell, including a non-human mammalian cell or human cell. In some aspects, the cell is a primate cell, including a macaque monkey cell. Also provided are compositions comprising the modified or chimeric HIV-1 virus of the invention as described above or herein and an excipient, such as a pharmaceutically acceptable excipient.

### BRIEF DESCRIPTION OF THE FIGURES

FIG. 1 is a graph illustrating the replication efficiency through 2 passages in pig tailed monkey peripheral blood mononuclear cells (pt mPBMC) of twelve HIV strains. Ten HIV-1 strains (AD-8, ELI, YU-2, Z2Z6, NL4-3, HXB2, MAL, LAI, and JRCSF), SHIV, and MD17 (MD17 nucleotide sequence is the known DH12 nucleotide sequence (GenBank Acc. No. AF069140) in which the nucleotide subsequence therein corresponding to HIV nef is replaced by the nucleotide sequence corresponding to SIV nef) were assayed by measuring RT activity. The maximum RT activity reached in SHIV infected cultures was in the range of 8000 cpm. The amount of input virus for the first pt mPBMC passage was normalized for RT activity. For infection of the second passage pt mPBMC supernatants from passage 1 showing peak RT activity were used. Days post-infection for each passage are labeled from the start of each infection of fresh pt mPBMC.

FIG. 2 is a schematic illustrating construction of the library of HIV-1 sequences with shuffled gag-pro-RT sequences. In this instance, these shuffled sequences were cloned into an infectious MD17 backbone. Such shuffled sequences can also be cloned into an infectious DH12 backbone.

FIG. 3 is a graph illustrating the replication efficiency in pt mPBMC as assayed by RT activity of four shuffled proviral libraries (1B3, 2B3, 2A3, 2A6) and a control mixture of the parental clones. Passages 4-7 are shown here; in the first three passages an alternating passaging regime was used where permissive huPBMC were inoculated with viral supernatants from infected pt mPBMC to rescue and amplify progeny viruses. The amplified viruses were then used to initiate the next round of infection in fresh pt mPBMC. Infection of the 4th passage of pt mPBMC was initiated using viruses amplified in huPBMC. Passages 5-7 were done without an intervening huPBMC amplification step. The maximum RT activity reached by SHIV infected cultures was in the range of 8000 cpm. For infection of pt mPBMC the supernatants from the previous passage showing peak RT activity were used. Days post-infection for each passage are labeled from the start of each infection of fresh pt mPBMC. The data show the emergence of a viral variant in library 1B3 that exhibits improved and persistent replication in pig-tailed (pt) macaque PMBC after extensive passaging of four gag-pro-RT shuffled libraries.

FIG. 4a is a graph illustrating a comparison of parallel infections of pt mPBMC of the improved viral variant 1B3, the best parent MD17, and the SHIV positive control. The maximum RT activity reached by SHIV infected cultures was in the range of 9000 cpm. The amount of input virus for the first pt mPBMC passage was normalized for RT activity. For infection of subsequent pt mPBMC passages supernatants from the previous passage showing peak RT activity were used. Days post-infection for each passage are labeled from the start of each infection of fresh pt mPBMC. The 1B3 viral variant showed improved replication in pt mPBMC compared to MD17 or DH12.

FIG. 4b illustrates the 1B3 out-competes MD17 (or DH12) during co-infection of pt mPBMC. Each lane shows the restriction pattern of the gag-pro-RT region of proviral sequences amplified from huPBMC infected with 1B3 only (lane 1), MD17 only (lane 2), co-infected with 1B3 and MD17 after passage 1 (lane 3), after passage 2 (lane 4), and after passage 3 (lane 5).

FIG. 5 illustrates a comparison of the replication kinetics of the improved clone 1.4 with that of the uncloned 1B3 virus as assayed by p24. The amount of input virus for the first pt mPBMC passage was normalized for p24. For infection of subsequent pt mPBMC passages supernatants from the previous passage showing peak virus production were used. Days post-infection for each passage are labeled from the start of each infection of fresh pt mPBMC. The results demonstrate that the cloned 1B3 (clone 1.4) virus exhibits the same replication kinetics in pt mPBMC as the uncloned 1B3 virus stock.

FIG. 6a illustrates a diagram of clone 1.4, illustrating the composition of the shuffled gag-pro-RT region in comparison to the parental strains and the non-silent point mutations in the remainder of the genome.

FIG. 6b illustrates the structure of seven recombinant chimeras comprising: 1) MD17 gag-pro-RT or DH12 gag-pro-RT in 1B3 clone 1.4 backbone (i.e., the “backbone” comprises nucleotide regions or segments other than the gag-pro-RT nucleotide segment); 2) MD17 int-env or DH12 int-env in 1B3 clone 1.4 backbone, 3) MD17 nef-LTR 1B3 clone 1.4 backbone, 4) DH12 nef-LTR in 1B3 clone 1.4 backbone, 5) 1B3 clone 1.4 gag-pro-RT in MD17 or DH12 backbone, 6) 1B3 clone 1.4 int-env in MD17 or DH12 backbone, and 7) 1B3 clone 1.4 nef-LTR in MD17 or DH12 backbone. The chimeras elucidate the relative contributions of various regions to the improved phenotype of clone 1.4.

FIG. 7a is a graph illustrating the replication efficiency in pt mPBMC for 2 passages as measured by p24 levels of recombinant or chimeric HIV-1 chimeras exchanging the gag-pro-RT region between the shuffled 1B3 clone 1.4 and the MD17 backbone. (DH12 can also serve as the backbone sequence.) The amount of input virus for the first pt mPBMC passage was normalized for p24. For infection of the second passage pt mPBMC supernatants from passage 1 showing peak virus production were used. Days post-infection for each passage are labeled from the start of each infection of fresh pt mPBMC.

FIG. 7b is a graph illustrating the replication efficiency in pt mPBMC for 2 passages as measured by p24 levels of recombinant or chimeric HIV-1 chimeras exchanging the int-env region between the shuffled 1B3 clone 1.4 and the MD17 backbone. (DH12 can also serve as the backbone sequence.) The amount of input virus for the first pt mPBMC passage was normalized for p24. For infection of the second passage pt mPBMC supernatants from passage 1 showing peak virus production were used. Days post-infection for each passage are labeled from the start of each infection of fresh pt mPBMC.

FIG. 7c is a graph illustrating the replication efficiency in pt mPBMC for 2 passages as measured by p24 levels of recombinant or chimeric HIV-1 chimeras exchanging the nef-LTR region between the shuffled 1B3 clone 1.4 and the MD17 backbone. (DH12 can also serve as the backbone.) The amount of input virus for the first pt mPBMC passage was normalized for p24. For infection of the second passage pt mPBMC supernatants from passage 1 showing peak virus production were used. Days post-infection for each passage are labeled from the start of each infection of fresh pt mPBMC.

FIG. 8 illustrates replication of the parental MD17 virus, 1B3 clone 1.4 and the MD17-1B3 chimeric viruses in huPBMC. The amount of input virus was normalized for RT activity.

FIG. 9 is a polynucleotide sequence alignment of clones 1.4 and P10.26 with the 10 parental HIV-1 sequences.

FIG. 10 is a polynucleotide sequence alignment of the seven recombinant or chimeric HIV-1 clones isolated from pool 1B3.

FIG. 11 is an amino acid sequence alignment of the nine recombinant or chimeric HIV-1 polypeptides encoded by each of the seven recombinant or chimeric HIV-1 clones isolated from pool 1B3.

### DETAILED DESCRIPTION

There exists a need for HIV-1 viral variants having improved replication properties and suitable animal models of HIV-1 infection. As noted above, present animal models, including chimpanzees and pig-tailed macaque monkeys, have limitations. For example, although HIV-1 infects chimpanzees, infected animals rarely develop any AIDS-lie symptoms. Furthermore, HIV-1 replicates poorly in pig-tailed macaque monkeys.

We addressed these limitations by using recursive sequence recombination and other diversity generation methods to produce and identify novel viruses that exhibit enhanced and efficient replication in macaque monkey cells and which comprise predominantly sequences derived from HIV-1, inclusive of all the HIV-1 structural genes. This was a formidable task, as the restrictions to productive HIV-1 replication in macaque monkey cells are poorly characterized. In rhesus mPBMC, HIV-1 replication appears to be blocked early in the viral life cycle (Shibata 1995, J. Gen. Virol., 76: 2723-2730; Himathongkham 1996, Virology, 219: 485-488). These blocks may involve the release of the virion core into the cytoplasm or occur at a step immediately prior to initiation of reverse transcription. Viral entry is not the limiting step as SHIVs can infect and replicate efficiently in macaque monkey cells. Determinants that restrict HIV-1 replication in mPBMC may reside in the sequences encompassing the 3′ half of the LTR and the gag-pol region (Shibata, Kawamura et al. 1991, J. Virol., 65: 3514-3520; Shibata 1995, J. Gen. Virol., 76: 2723-2730). Chackerian et al. (Chackerian 1997, J. Virol., 71: 3932-3939) reported that replication of HIV-1 in macaque monkey cells expressing human CD4 can be blocked at several steps; certain macrophage-tropic and primary HIV-1 isolates are restricted at a step subsequent to reverse transcription but prior to migration of the pre-integration complex to the nucleus while other isolates are blocked prior to reverse transcription. However, expressing the appropriate human co-receptor on the surface of the rhesus macaque monkey cells alleviates the replication blocks (Chackerian 1997, J. Virol., 71: 3932-3939). In contrast to the complete restriction seen in rhesus macaque monkey cells, pig-tailed macaques are semi-permissive for HIV-1 replication. Some HIV-1 strains can infect T-lymphocytes from this species but replicate at very low levels (Gartner 1994, AIDS Res. Hum. Retroviruses, 10: S129-133; Gartner 1994, J. Med. Primatol., 23: 155-163). The blocks here are also poorly defined and appear to act at stages after reverse transcription (Kimball 1998, J. Med. Primatol., 27: 99-103).

The undefined nature of the replication blocks and the intrinsic complexity of the HIV-1 genome preclude the rational ‘engineering’ of the virus to replicate efficiently in pig-tailed macaques. Attempts to adapt HIV-1 by serial passaging in pig-tailed macaques have not been successful (Agy et al., 1997, Virology, 238: 336-343) as replication is not sufficiently robust to favor the evolution of improved variants. Furthermore, efficient CTL responses in HIV-1 infected pig-tailed macaques limit virus replication and eventually clear the virus (Kent 1995, J. Clin. Invest., 95: 248-256; Kent 1997, J. Infect. Dis., 176: 1188-1197).

In light of the barriers to rational and adaptive approaches to designing novel HIV-1 viruses that replicate efficiently in pig-tailed macaques with, we applied recursive sequence recombinant (e.g., DNA shuffling) to kindle the process of virus evolution. In DNA shuffling, homologous sequences can be recombined in vitro by random fragmentation, followed by cycles of reassembly. This generates a pool of diverse, recombinant sequences that is then screened for novel and improved properties. The power of DNA shuffling to evolve dramatically improved phenotypes by efficiently permuting functional sequences from multiple parents has been demonstrated in many systems (Crameri 1998, Nature, 391: 288-291; Chang 1999, Nat. Biotechnol., 17: 793-797; Ness 1999, Nat. Biotechnol., 17: 893-896). We have also shown that shuffling can enhance the inherently high evolutionary potential of retroviruses. Under different selective pressures, a library of murine leukemia viruses (MLV) containing shuffled envelopes yielded chimeras with a new tropism (Soong 2000, Nat. Genetics, 25: 436-439), as well as dramatically increased mechanical stabilities (Powell 2000, Nat. Biotechnol., 18: 1279-1282). Here we generated diverse libraries of HIV-1 recombinants using DNA shuffling. By selectively passaging these libraries, we evolved multiple viral species that can now replicate efficiently and sustainably in pt mPBMC.

The invention provides recombinant nucleic acids, polypeptides, vectors, and compositions corresponding to novel recombinant or chimeric HIV-1 nucleic acids that encode recombinant or chimeric HIV-1 viruses that exhibit enhanced replication in non-human cells. The invention also provides recombinant or chimeric HIV-1 viruses that exhibit enhanced replication in non-human mammalian cells compared to replication of a HIV-1 virus (e.g., wild-type HIV-1 virus) in non-human mammalian cells. In particular, the invention provides recombinant or chimeric HIV-1 viruses that exhibit enhanced replication in macaque monkey cells, including, e.g., but not limited to pig-tailed macaque monkey cells, compared to replication of an HIV-1 virus in said macaque monkey cells. The invention also provides cells and compositions comprising said recombinant or chimeric HIV-1 viruses. Also provided are non-human primates that comprise these recombinant or chimeric HIV-1 viruses. In particular, the invention provides non-human primates that comprise the recombinant or chimeric HIV-1 viruses, wherein this non-human primates exhibits at least one symptom of HIV infection. The invention further provides novel recombinant or chimeric HIV-1 polypeptides, and compositions comprising these polypeptides; methods of screening for agents that inhibit and/or treat HIV infection; and chimeric or recombinant nucleic acids that comprise recombinant or chimeric HIV-1 nucleic acid and wild-type or known HIV-1 nucleic acid.

A primary advantage of the recombinant or chimeric HIV-1 viruses of the invention (e.g., an HIV-1 virus comprising at least one recombinant or chimeric HIV-1 nucleic acid of the invention or at least one chimeric or recombinant polypeptide of the invention as described above or herein) is that the recombinant viruses replicate efficiently in macaque cells and comprise HIV-1 nucleic acid and/or polypeptide sequences derived from HIV-1, including, e.g., some or all the HIV-1 structural genes. Chimeric or recombinant nucleic acids of the invention include those comprising a polynucleotide sequence that has at least about 80, 85, 88, 90, 91, 92, 93, 94, 95, 96, 97, 98.5, 99, 99.5% or 100% sequence identity to at least one polynucleotide sequence selected from the group of SEQ ID NO:1 to SEQ ID NO:7, or a complementary polynucleotide sequence thereof. Some such nucleic acids produce a chimeric or recombinant human immunodeficiency virus type 1 (HIV-1) variant that exhibits enhanced replication in macaque monkey cells compared to the replication of an HIV-1 virus in macaque monkey cells.

Chimeric or recombinant polypeptides of the invention include those encoded by a chimeric or nucleic acid of the invention. Also included are chimeric or recombinant polypeptides that comprise an amino acid sequence that has at least about 80, 85, 88, 90, 91, 92, 93, 94, 95, 96, 97, 98.5, 99, 99.5%, or 100% amino acid sequence identity to at least one amino acid sequence selected from the group of SEQ ID NOS:8-39, or to a polypeptide fragment thereof, wherein a virus comprising such polypeptide fragment that exhibits enhanced replication in macaque monkey cells compared to the replication of a WT HIV-1 virus in macaque monkey cells (e.g., pig-tailed macaque monkey cells). Also included are chimeric or recombinant polynucleotides that encode a polypeptide having at least about 80, 85, 88, 90, 91, 92, 93, 94, 95, 96, 97, 98.5, 99, 99.5%, or 100% amino acid sequence identity to at least one amino acid sequence selected from the group of SEQ ID NOS:8-39, or that encode a polypeptide fragment thereof, wherein a virus comprising such polypeptide fragment that exhibits enhanced replication in macaque monkey cells compared to the replication of a WT HIV-1 virus in macaque monkey cells (e.g., pig-tailed macaque monkey cells).

As explained above, there is a great need for an animal model of HIV-1 infection in humans. The recombinant or chimeric HIV-1 viruses, nucleic acids, and polypeptides (and fragments thereof) of the invention are useful in developing non-human primate models (e.g., macaque monkey models) of HIV-1 infection and AIDS. These non-human primate models are useful for studying HIV-1 infection, AIDS associated symptoms and disorders, and the AIDS process of infection. These animal models are also useful for testing and studying potential vaccines and antiviral drugs.

In addition, the recombinant or chimeric HIV-1 viruses of the invention are each useful as challenge viruses for HIV-1 vaccines, including subunit vaccines for gag, env, rev, tat, etc. Alternatively, the recombinant or chimeric HIV-1 viruses, nucleic acids, and/or polypeptides of the invention are each useful as HIV-1 vaccines, alone or in combination with one another e.g., viral, DNA, or protein vaccines, including as subunit vaccines for humans and other mammals.

Uses of the recombinant or chimeric HIV-1 viruses and nucleic acids of the invention also include uses for the development of diagnostic assays.

The recombinant or chimeric HIV-1 nucleic acids of the invention are also useful as substrates for further modification to produce, e.g., an attenuated HIV-1 version. For example, the nef gene can be deleted and the resulting attenuated HIV-1 used as a vaccine in macaque monkey models or humans. In another example, sequences from other HIV-1 clades are transplanted into the recombinant or chimeric HIV-1 nucleic acids of the invention to produce novel HIV-1 viruses that are useful in creating a vaccine, a challenge model, or macaque monkey models. In a further example, the recombinant or chimeric HIV-1 nucleic acids of the invention are evolved further in vitro and in vivo by recursive sequence recombination of nucleic acid segments or other diversity generation methods (e.g., DNA shuffling), and passaging and screening for improved robustness and/or replication and pathogenicity in non-human mammals, e.g., macaque monkeys, e.g., pig-tailed macaque monkeys.

Definitions

Unless otherwise defined herein or below in the remainder of the specification, all technical and scientific terms used herein have the same meaning as commonly understood by those of ordinary skill in the art to which the invention belongs.

Amino acid sequence: An “amino acid sequence” is the primary sequence of amino acid residues of an amino acid polymer, e.g., a polypeptide or protein or peptide, or a character string representing an amino acid polymer, depending on context.

Complementary: As used herein, the term “complementary” refers to the capacity for precise pairing between two nucleotides. Thus, if a nucleotide at a given position of a nucleic acid molecule is capable of hydrogen bonding with a nucleotide of another nucleic acid molecule, then the two nucleic acid molecules are considered to be complementary to one another at that position. The term “substantially complementary” describes sequences that are sufficiently complementary to one another to allow for specific hybridization under stringent hybridization conditions. The term “perfectly complementary” refers to sequences in which there are no mismatched nucleotides (i.e., each nucleotide in both sequences can hydrogen bond with a complementary nucleotide in the other sequence). One such sequence is said to be the “perfect complement” of the other.

DH12: The term DH12 refers to an HIV-1 virus encoded by the polynucleotide sequence with GenBank accession No. AF069140 (SEQ ID NO:40) and the amino acid sequence of GenBank Accession No. AF069139, each of which sequence is incorporated herein by reference in its entirety for all purposes. The term also refers to an HIV-1 virus encoded by the polynucleotide sequence with GenBank accession no: AF069140 (SEQ ID NO:40) and comprising the nucleotides GC at positions 8478/8479 instead of the nucleotides CG, thereby producing an amino acid residue S instead of an amino acid residue T in the Env coding region and amino acid residues EP instead of amino acid residues DA in the Rev coding region.

Amount: The term “amount sufficient” means a dosage or amount effective to produce a desired result. The desired result can comprise an objective or subjective improvement in the subject receiving the dosage or amount. “An amount sufficient to cause HIV infection” includes, e.g., a dose corresponding to 300 Tissue culture infectious dose 50% (TCID₅₀) to 5×10⁵ TCID₅₀.

Enhanced replication: In reference to a recombinant or modified HIV-1 virus that exhibits “enhanced replication,” the term refers to at least the following three phenotypes: 1) a recombinant or modified HIV-1 virus that grows to a higher titer than a WT or known HIV-1 virus; 2) a recombinant or modified HIV-1 virus that replicates for a longer period of time through one or more passages, e.g., in tissue culture and/or in whole animal, than a WT or known HIV-1 virus; and/or 3) a recombinant or modified HIV-1 virus that grows at a faster rate than a WT or known HIV-1 virus.

Excipient: An “excipient” or “carrier” is an inert substance used as a vehicle or diluent for a composition, e.g., for a drug. The term “pharmaceutically acceptable excipient” means an excipient suitable for pharmaceutical use in a subject, including an animal or human.

Gene: The term “gene” broadly refers to any segment of DNA, or portion or fragment thereof, associated with a biological function. Genes include coding regions and/or regulatory sequences required for their expression. Genes also include non-expressed DNA nucleic acid segments that, e.g., form recognition sequences for other proteins (e.g., promoter, enhancer, or other regulatory regions). Genes can be obtained from a variety of sources, including cloning from a source of interest or synthesizing from known or predicted sequence information, and can include sequences designed to have desired parameters. “Nucleic acid derived from a gene” refers to a nucleic acid for whose synthesis the gene, or a subsequence thereof, has ultimately served as a template. Thus, an mRNA, a cDNA reverse transcribed from an mRNA, an RNA transcribed from that cDNA, a DNA amplified from the cDNA, an RNA transcribed from the amplified DNA, etc., are all derived from the gene and detection of such derived products is indicative of the presence and/or abundance of the original gene and/or gene transcript in a sample.

HIV infection: As used herein the term “HIV infection” refers to infection of at least one cell by an HIV-1 virus, e.g., in vitro, in vivo, or ex vivo. HIV infection is detected by assays well-known to one of skill in the art, including, for example: the presence of viral antibodies to viral antigens; measurement of plasma RNA levels; measurement of proviral DNA; measurement of plasma antigen levels

Human immunodeficiency virus type 1: The term “Human immunodeficiency virus type 1” or “HIV-1” refers to the retrovirus recognized as the agent that induces acquired immunodeficiency syndrome (AIDS) in humans. An HIV-1 virus includes the genes for gag, pol (including protease (pro) and reverse transcriptase (RT)), vive, vapor, tat, rev, put, env, and nef.

Hybridizes: The term “hybridizes,” “hybridize,” “hybridizing,” or “hybridization” refers to the association of nucleic acids, typically in solution. Nucleic acids hybridize due to a variety of well-characterized physic-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes, part I, chapter 2, “Overview of principles of hybridization and the strategy of nucleic acid probe assays,” (Elsevier, New York), as well as in Ausubel, supra. Hames and Higgins (1995) Gene Probes 1, IRL Press at Oxford University Press, Oxford, England, (“Hames and Higgins 1”) and Hames and Higgins (1995) Gene Probes 2, IRL Press at Oxford University Press, Oxford, England (“Hames and Higgins 2”) provide details on the synthesis, labeling, detection and quantification of DNA and RNA, including oligonucleotides.

The phrase “hybridizing specifically to” refers to the binding, duplexing, or hybridizing of a molecule only to a particular nucleotide sequence under stringent conditions when that sequence is present in a complex mixture (e.g., total cellular DNA or RNA). “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. An indication that two nucleic acid sequences are substantially identical is that the two molecules hybridize to each other under at least stringent conditions.

In the context of nucleic acid hybridization “washing” refers to the removal of unhybridized nucleic acid material. This can be done with a series of washes, the stringency of which can be adjusted depending upon the desired results. Low stringency washing conditions (e.g., using higher salt and lower temperature) increase sensitivity, but can product nonspecific hybridization signals and high background signals. Higher stringency conditions (e.g., using lower salt and higher temperature that is closer to the hybridization temperature) lowers the background signal, typically with only the specific signal remaining. See Rapley, R. and Walker, J. M. eds., Molecular Biomethods Handbook (Humana Press, Inc. 1998) (hereinafter “Rapley and Walker”), which is incorporated herein by reference in its entirety for all purposes.

The “thermal melting point” or “Tm” of a nucleic acid duplex is the temperature (under defined ionic strength and pH) at which the duplex is 50% denatured under the given conditions and represents a direct measure of the stability of the nucleic acid hybrid. Thus, the Tm corresponds to the temperature corresponding to the midpoint in transition from helix to random coil; it depends on length, nucleotide composition, and ionic strength for long stretches of nucleotides. The Tm of a DNA-DNA duplex can be estimated using the following equation (1):

(1) Tm (° C.)=81.5° C.+16.6 (log10M)+0.41 (% G+C)−0.72 (% f)−500/n.

The Tm of an RNA-DNA duplex can be estimated by equation (2) as follows:

(2) Tm (° C.)=79.8° C.+18.5 (log10M)+0.58 (% G+C)−11.8(% G+C)2−0.56 (% f)−820/n.

In both equations (1) and (2), M is the molarity of the monovalent cations (usually Na+), (% G+C) is the percentage of guanosine (G) and cystosine (C) nucleotides, (% f) is the percentage of formamide and n is the number of nucleotide bases (i.e., length) of the hybrid. See Rapley and Walker, supra. Equations 1 and 2 typically accurate only for hybrid duplexes longer than about 100-200 nucleotides. Id. The Tm of nucleic acid sequences shorter than 50 nucleotides can be calculated as follows: Tm (° C.)=4(G+C)+2(A+T), where A (adenine), C, T (thymine), and G are the numbers of the corresponding nucleotides.

Isolated: A nucleic acid, polypeptide, or other object species is “isolated” when it is partially or completely separated from components with which it is normally associated (other peptides, polypeptides, proteins (including complexes, e.g., polymerases and ribosomes which can accompany a native sequence), nucleic acids, cells, synthetic reagents, cellular contaminants, cellular components, etc.), e.g., such as from other components with which it is normally associated in the cell from which it was originally derived. A nucleic acid, polypeptide, or other object species is isolated when it is partially or completely recovered or separated from other components of its natural environment such that it is the predominant species present in a composition, mixture, or collection of components (i.e., on a molar basis it is more abundant than any other individual species in the composition). In preferred embodiments, the preparation comprises of more than about 70% or 75%, typically more than about 80%, or preferably more than about 90% of the isolated species.

In one aspect, a “substantially pure” or “isolated” nucleic acid, polypeptide, protein, or other object species also means where the object species comprises at least about 50, 60, or 70 percent by weight (on a molar basis) of all macromolecular species present. A substantially pure or isolated other object species can also comprise at least about 80, 90, or 95 percent by weight of all macromolecular species present in the composition. An isolated object species can also be purified to essential homogeneity (contaminant species cannot be detected in the composition by conventional detection methods) wherein the composition comprises essentially of derivatives of a single macromolecular species.

The term “purified” generally denotes that a nucleic acid, polypeptide, or protein gives rise to essentially one band in an electrophoretic gel. It typically means that the nucleic acid, polypeptide, or protein is at least about 50% pure, 60% pure, 70% pure, 75% pure, more preferably at least about 85% pure, and most preferably at least about 99% pure. The term “isolated nucleic acid” can refer to a nucleic acid that is not immediately contiguous with both of the sequences with which it is immediately contiguous (i.e., one at the 5′ and one at the 3′ end) in the naturally occurring genome of the organism from which the nucleic acid is derived. Thus, this term includes, e.g., a cDNA or a genomic DNA fragment produced by polymerase chain reaction (PCR) or restriction endonuclease treatment, whether such cDNA or genomic DNA fragment is incorporated into a vector, integrated into the genome of the same or a different species than the organism, including, e.g., a virus, from which it was originally derived, linked to an additional coding region to form a hybrid gene encoding a chimeric polypeptide, or independent of any other DNA sequences. The DNA can be double-stranded or single-stranded, sense or antisense.

Library: A “library” of nucleic acids includes at least 2 different nucleic acids, and preferably at least about 5, 10, 20, 50, 10², 10³, 10⁴, 10⁵, 10⁶, 10⁷ or more different nucleic acids.

Non-human mammal: As used herein includes the term “non-human mammal” includes, but is not limited to, a non-human primate, pig, cow, goat, cat, rabbit, rat, guinea pig, hamster, horse, monkey, or sheep. A non-human primate includes, but is not limited to, a chimpanzee, baboon, orangutan, monkey, etc. A monkey includes, but is not limited to, all species of macaque monkey, e.g., pig-tailed macaque, rhesus macaque, and cyngologous macaque.

Nucleic acid: The term “nucleic acid” includes a polymer of nucleic acid residues or nucleotides (A, C, T, U, G, etc. or naturally occurring or artificial nucleotide analogues), or a character string representing a nucleic acid, depending on context. Nucleic acids includes deoxyribonucleotides (DNA) or ribonucleotides (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. The term nucleic acid is used interchangeably with the term “polynucleotides” and encompasses genes, cDNA, and mRNA encoded by a gene.

Polynucleotide sequence: A “polynucleotide sequence” is the primary sequence of nucleotides, e.g., nucleic acid residues, of a nucleic acid, e.g., a DNA or an RNA, or a character string representing a nucleic acid, depending on context. Unless otherwise indicated, the polynucleotide sequence of a particular nucleic acid also implicitly encompasses conservatively modified variants thereof (e.g., degenerate codon substitutions) and complementary sequences and as well as the sequence explicitly indicated. Either the given nucleic acid or the complementary nucleic acid can be determined from any specified polynucleotide sequence.

Polypeptide: A “polypeptide” is a polymer of amino acid residues (alanine, threonine, etc. and/or naturally occurring or artificial amino acid analogues), including, e.g., a protein, peptide, polypeptide, etc. The nucleic acid that encodes a polypeptide, or the complementary nucleic acid thereof, can be determined from any specified amino acid sequence. Given the degeneracy of the genetic code, one or more nucleic acids, or the complementary nucleic acids thereof, can encode a specific polypeptide.

Recombinant: The term “recombinant” when used with reference, e.g., to a virus, cell, vector, nucleic acid, or polypeptide typically indicates that the virus, cell, vector, nucleic acid has been modified by the introduction of a heterologous (or foreign) nucleic acid or the alteration of a native nucleic acid, or that the polypeptide has been modified by the introduction of a heterologous amino acid or the alteration of a native amino acid, or that the cell is derived from a cell so modified.

In another aspect, the term “recombinant” when used with reference to a cell indicates that the cell replicates a heterologous nucleic acid, or expresses a polypeptide encoded by a heterologous nucleic acid. “Recombinant cells” express nucleic acid sequences (e.g., genes) that are not found in the native (non-recombinant) form of the cell or express native nucleic acid sequences (e.g., genes) that would be abnormally expressed, under-expressed, or not expressed at all. Recombinant cells can contain genes that are not found within the native (non-recombinant) form of the cell. Recombinant cells can also contain genes found in the native form of the cell wherein the genes are modified and re-introduced into the cell by artificial means. The term “recombinant cells” also encompasses cells that contain a nucleic acid endogenous to the cell that has been modified without removing the nucleic acid from the cell; such modifications include those obtained by gene replacement, site-specific mutation, and related techniques.

The terms “recombinant nucleic acid” or a “recombinant polypeptide” encompass a non-naturally occurring nucleic acid or polypeptide that includes polynucleotide or amino acid sequences, respectively, from more than one source nucleic acid or polypeptide, which source nucleic acid or polypeptide can be a naturally occurring nucleic acid or polypeptide, or can itself have been subjected to mutagenesis or other type of modification. A nucleic acid or polypeptide can be deemed “recombinant” when it is artificial or engineered, or derived from an artificial or engineered polypeptide or nucleic acid. A recombinant nucleic acid can be made by the combination (e.g., artificial combination) of at least two segments of sequence that are not typically included together, not typically associated with one another, or are otherwise typically separated from one another. A recombinant nucleic acid can comprise a nucleic acid molecule formed by the joining together or combination of nucleic acid segments from different sources and/or artificially synthesized. A recombinant polypeptide often refers to a polypeptide (or protein) that results from a cloned or recombinant nucleic acid or gene. The source nucleic acids or polypeptides from which the different polynucleotide or amino acid sequences are derived are sometimes homologous (i.e., have, or encode a polypeptide that encodes, the same or a similar structure and/or function), and are often from different isolates, serotypes, strains, species, of organism or from different disease states, for example.

The term “recombinantly produced” refers to an artificial combination usually accomplished by either chemical synthesis means, recursive sequence recombination of nucleic acid segments or other diversity generation methods (such as, e.g., shuffling) of nucleotides, or manipulation of isolated segments of nucleic acids, e.g., by genetic engineering techniques known to those of ordinary skill in the art. “Recombinantly expressed” typically refers to techniques for the production of a recombinant nucleic acid in vitro and transfer of the recombinant nucleic acid into cells in vivo, in vitro, or ex vivo where it can be expressed or propagated.

Sequence identity: With regard to polynucleotide sequences, the term “sequence identity” means that two polynucleotide sequences are identical (i.e., on a nucleotide-by-nucleotide basis) over a window of comparison. The term “percentage of sequence identity” or “percentage of sequence similarity” is calculated by comparing two optimally aligned sequences over the window of comparison, determining the number of positions at which the identical residues occur in both nucleotide sequences to yield the number of matched positions, dividing the number of matched positions by the total number of positions in the window of comparison (i.e., the window size), and multiplying the result by 100 to yield the percentage of sequence identity (or percentage of sequence similarity). With regard to amino acid sequences, the term sequence identity likewise means that two amino acid sequences are identical (on an amino acid-by-amino acid basis) over a window of comparison, and a percentage of amino acid residue sequence identity (or percentage of amino acid residue sequence similarity), also can be calculated. Maximum correspondence can be determined by using one of the sequence algorithms described herein (or other algorithms available to those of ordinary skill in the art) or by visual inspection. The term sequence identity is described in further detail below.

Simian immunodeficiency virus: A “simian immunodeficiency virus” (SIV) is a lentivirus that induces acquired immunodeficiency syndrome in monkeys and apes (SAIDS). SIV includes the subgroups SIV-1 and SIV-2.

Stringent conditions: “Stringent” hybridization and wash conditions in the context of nucleic acid hybridization experiments, such as Southern and northern hybridizations, are sequence dependent and are different under different environmental parameters. An extensive guide to the hybridization of nucleic acids is found in Tijssen (1993), supra, and in Hames and Higgins 1 and Hames and Higgins 2, supra. Target sequences that are closely related or identical to the nucleotide sequence of interest (e.g., “probes”) can be identified under highly stringent conditions. Lower stringency conditions are appropriate for sequences that are less complementary. See, e.g., Rapley and Walker, supra. An example of stringent hybridization conditions for hybridization of complementary nucleic acids which have more than 100 complementary residues on a filter in a Southern or northern blot is 50% formamide (or formalin) with 1 mg of heparin at 42° C., with the hybridization being carried out overnight. An example of stringent wash conditions is a 0.2×SSC wash at 65° C. for 15 minutes (see Sambrook, supra, for a description of SSC buffer). Often, a high stringency wash is preceded by a low stringency wash to remove background probe signal. An example low stringency wash is 2×SSC at 40° C. for 15 minutes. In general, a signal to noise ratio of 5× (or higher) than that observed for an unrelated probe in the particular hybridization assays indicates detection of a specific hybridization.

For purposes of the present invention, generally, “highly stringent” hybridization and wash conditions are selected to be about 5° C. lower than the Tm for the specific sequence at a defined ionic strength and pH (as noted below, highly stringent conditions can also be referred to in comparative terms). “Very stringent” hybridization and wash conditions are selected to be equal to the Tm for a particular probe. Stringent hybridization (and e.g., highly stringent, “ultra-high stringency”, or “ultra-ultra high stringency” conditions) and wash conditions can easily be determined empirically for any test nucleic acid. For example, in determining highly stringent hybridization and wash conditions, the hybridization and wash conditions are gradually increased (e.g., by increasing temperature, decreasing salt concentration, increasing detergent concentration and/or increasing the concentration of organic solvents, such as formalin, in the hybridization or wash), until a selected set of criteria are met. Additionally, for distinguishing between duplexes with sequences of less than about 100 nucleotides, a TMACl hybridization procedure known to those of ordinary skill in the art can be used. See, e.g., Sorg, U. et al., 1 Nucleic Acids Res (Sep. 11, 1991) 19(17), incorporated herein by reference in its entirety for all purposes.

Subsequence: A “subsequence” or “fragment” is any portion of an entire sequence (e.g., polynucleotide or amino acid sequence), up to and including the complete sequence.

Symptom of HIV infection: As used herein, the term “symptom of HIV infection” includes the broad range of symptoms commonly associated with HIV-1 infection of humans, including the onset of immunodeficiency and/or symptoms associated with AIDS, and are well-known to one of skill in the art. Symptoms of HIV-1 infection are assessed by measurement over time of various virological, immunological and clinical parameters. Symptoms include, but are not limited to, e.g., high levels of viral load, loss of CD4 positive T cells, and a variety of clinical symptoms, such as wasting, opportunistic infections, and neurological disorders.

Substantially an entire length of a polynucleotide or amino acid sequence: “Substantially an entire length of a polynucleotide or amino acid sequence” refers to at least 50%, at least 60%, generally at least 70%, generally at least 80% or 85%, or typically at least 90%, 91%, 92%, 93%, 94%, 95%, 96%, 96.5%, 97%, 97.5%, 98%, 98.5%, 99%, or 99.5% or more of a polynucleotide or amino acid sequence.

Vector: A “vector” is a component or composition for facilitating transfer e.g., transduction, transfection, or infection, of a selected nucleic acid in a cell. Vectors include, e.g., plasmids, cosmids, phage, viruses, YACs, bacteria, poly-lysine, etc. An “expression vector” is a nucleic acid construct or sequence, generated recombinantly or synthetically, with a series of specific nucleic acid elements that permit transcription of a particular nucleic acid in a host cell. The expression vector can be part of a plasmid, virus, or nucleic acid fragment. The expression vector typically comprises a nucleic acid to be transcribed (i.e., a transgene) operably linked to a promoter.

A nucleic acid is “operably linked” when it is placed into a functional relationship with another nucleic acid sequence. For instance, a promoter or enhancer is operably linked to a coding sequence if it directs or increases the transcription of the coding sequence. A nucleic acid is said to “promote the expression” of an operably linked coding sequence if the nucleic acid acts as a promoter (i.e., directs transcription) or as an enhancer (i.e., increases transcription). “Operably linked” means that the DNA sequences being linked are optionally contiguous and, where necessary to join two protein coding regions, contiguous and in reading frame. However, since enhancers generally function when separated from the promoter by several kilobases and intronic sequences can be of variable lengths, some polynucleotide elements can be operably linked but not contiguous.

Viremia: The term “viremia” refers to the presence of viruses in the bloodstream.

Virus-like particle: A “virus like particle” or “VLP” is an assembly if one or more viral proteins that is devoid of a viral genome. For example, a cell expressing, e.g., an HIV-1 gag gene alone can produce a VLP.

Discussion

Generally, the nomenclature used hereafter and the laboratory procedures in cell culture, molecular genetics, molecular biology, nucleic acid chemistry, and protein chemistry described below are those well known and commonly employed by those of ordinary skill in the art. Standard techniques, such as described in Sambrook et al., Molecular Cloning—A Laboratory Manual (2nd Ed.), Vols. 1-3, Cold Spring Harbor Laboratory, Cold Spring Harbor, N.Y., 1989 (hereinafter “Sambrook”) and Current Protocols in Molecular Biology, F. M. Ausubel et al., eds., Current Protocols, a joint venture between Greene Publishing Associates, Inc. and John Wiley & Sons, Inc. (1994, supplemented through 1999) (hereinafter “Ausubel”), are used for recombinant nucleic acid methods, nucleic acid synthesis, cell culture methods, and transgene incorporation, e.g., electroporation, injection, gene gun, impressing through the skin, and lipofection. Generally, oligonucleotide synthesis and purification steps are performed according to specifications. The techniques and procedures are generally performed according to conventional methods in the art and various general references that are provided throughout this document. The procedures therein are believed to be well known to those of ordinary skill in the art and are provided for the convenience of the reader.

Recombinant or Chimeric HIV-1 Polynucleotide Sequences

The invention provides isolated, chimeric or recombinant or chimeric HIV-1 polynucleotide sequences that encode recombinant or chimeric HIV-1 viruses and polypeptides, fragments of HIV-1 polypeptides, related fusion polypeptides or proteins, or functional equivalents thereof, and are collectively referred to herein as “recombinant or chimeric HIV-1 nucleic acids.” As described in greater detail below, the recombinant or chimeric HIV-1 nucleic acids of the invention are useful in a variety of applications, including, but not limited to, in recombinant production of the recombinant or chimeric HV-1 polypeptides of the invention; to evolve further recombinant or chimeric HIV-1 nucleic acids; and as diagnostic probes for the presence of complementary or partially complementary nucleic acids (including for detection of natural HIV-1 nucleic acids). The recombinant or chimeric HIV-1 nucleic acids are useful for producing recombinant or chimeric HIV-1 viruses that exhibit enhanced replication in pig tailed macaque cells. The recombinant or chimeric HIV-1 nucleic acids are also useful for producing animal models for HIV-1 infection.

As described in more detail in the Examples below, the recombinant or chimeric HIV-1 nucleic acids of the invention were obtained by performing DNA shuffling on the gag-pro-RT region of the HIV-1 genome, using 11 parental HIV-1 genomes as the starting polynucleotide sequences. The resulting pools of gag-pro-RT fragments were subcloned into a modified DH12 (SEQ ID NO:40) backbone, where the modification includes a change of CG to GC at nucleotide residue positions 8478 and 8479. One pool of virus (1B3) demonstrated enhance replication in pig-tailed macaque monkey cells. Seven clones were isolated from pool 1B3; the polynucleotide sequences of these seven clones are described in SEQ ID NO:1-7. In a preferred embodiment, the isolated, chimeric or recombinant nucleic acid comprises a polynucleotide sequence selected from the group of SEQ ID NO:1 to SEQ ID NO:7 or a complementary polynucleotide sequence thereof.

The invention also comprises nucleic acid fragments of these recombinant or chimeric HIV-1 polynucleotide sequences, as well as variants including an insertion, substitution, and/or deletion of one or more nucleotides and nucleic acids that are otherwise modified. Preferably, recombinant or chimeric HIV-1 viruses comprising these fragments, nucleotide sequence variants, and modified forms of the disclosed polynucleotide sequences exhibit enhanced replication as described herein.

Accordingly, one aspect of the invention is an isolated, chimeric or recombinant nucleic acid comprising a polynucleotide sequence that has at least about 90%, at least about 91%, at least about 92%, at least about 93%, at least about 94%, at least about 95%, at least about 96%, at least about 97%, at least about 98% at least about 99%, at least about 99.5% sequence identity to at least one polynucleotide sequence selected from the group of SEQ ID NO:1 to SEQ ID NO:7 or to a complementary polynucleotide sequence thereof. In one embodiment, this isolated, chimeric or recombinant nucleic acid produces a recombinant or modified human immunodeficiency virus type 1 (HIV-1) that exhibits enhanced replication in macaque monkey cells compared to replication of an HIV-1 (e.g., a WT or known) virus in said macaque monkey cells. Enhanced replication includes, e.g., growth to a higher titer than a wild-type or known HIV-1 virus; replication for a longer period of time through one or more passages, e.g., in tissue culture and/or in whole animal, than a WT or known HIV-1 virus; and/or growth at a faster rate than a WT or known HIV-1 virus. Any species of macaque monkey is included, e.g., rhesus macaque, pig-tailed macaque, and the like.

In another embodiment, this isolated, chimeric or recombinant nucleic acid comprises a first polynucleotide subsequence comprising an HIV-1 nef gene replaced with second polynucleotide subsequence comprising a simian immunodeficiency virus (SIV) nef gene.

In another embodiment, the invention provides recombinant or chimeric HIV-1 polynucleotides encoding novel recombinant or chimeric HIV-1 polypeptides. Accordingly, the invention provides an isolated, chimeric or recombinant nucleic acid comprising a polynucleotide sequence selected from the group of: (a) a polynucleotide sequence that encodes nine polypeptides, said nine polypeptides comprising the amino acid sequences of SEQ ID NO:8 to SEQ ID NO:16, respectively, or a complementary polynucleotide sequence thereof; (b) a polynucleotide sequence that encodes nine polypeptides, said nine polypeptides comprising the amino acid sequences of SEQ ID NO:17, SEQ ID NO:9, SEQ ID NO:18, SEQ ID NO:11, SEQ ID NO:12, SEQ ID NO:13, SEQ ID NO:15, SEQ ID NO:19, or a complementary polynucleotide sequence thereof; (c) a polynucleotide sequence that encodes nine polypeptides, said nine polypeptides comprising the amino acid sequences of SEQ ID NO:8, SEQ ID NO:20, SEQ ID NO:10, SEQ ID NO:1, SEQ ID NO:12, SEQ ID NO:13, SEQ ID NO:14, SEQ ID NO:14, and SEQ ID NO:21, or a complementary polynucleotide sequence thereof; (d) a polynucleotide sequence that encodes nine polypeptides, said nine polypeptides comprising the amino acid sequences of SEQ ID NO:22, SEQ ID NO:23, SEQ ID NO:10, SEQ ID NO:11, SEQ ID NO:12, SEQ ID NO:24, SEQ ID NO:14, SEQ ID NO:25, and SEQ ID NO:26, or a complementary polynucleotide sequence thereof; (e) a polynucleotide sequence that encodes nine polypeptides, said nine polypeptides comprising the amino acid sequences of SEQ ID NO:27, SEQ ID NO:9, SEQ ID NO:28, SEQ ID NO:11, SEQ ID NO:12, SEQ ID NO:13, SEQ ID NO:14, SEQ ID NO:29, and SEQ ID NO:30, or a complementary polynucleotide sequence thereof; (f) a polynucleotide sequence that encodes nine polypeptides, said nine polypeptides comprising the amino acid sequences of SEQ ID NO:8, SEQ ID NO:9, SEQ ID NO:31, SEQ ID NO:11, SEQ ID NO:12, SEQ ID NO:13, SEQ ID NO:14, SEQ ID NO:32, and SEQ ID NO:33, or a complementary polynucleotide sequence thereof; and (g) a polynucleotide sequence that encodes nine polypeptides, said nine polypeptides comprising the amino acid sequences of SEQ ID NO:34, SEQ ID NO:35, SEQ ID NO:10, SEQ ID NO:36, SEQ ID NO:12, SEQ ID NO:13, SEQ ID NO:37, SEQ ID NO:38, and SEQ ID NO:39, or a complementary polynucleotide sequence thereof.

In one embodiment, the isolated, chimeric or recombinant nucleic acids of the invention include a DNA polynucleotide sequence. In other embodiments, the isolated, chimeric or recombinant nucleic acids of the invention include replacement of every thymine with uracil. In a preferred embodiment, the isolated, chimeric or recombinant nucleic acids of the invention comprise nucleic acid residue 530 to at least about nucleic acid residue 9859, wherein each thymine is replaced by a uracil. The invention also provides an isolated, chimeric or recombinant RNA transcribed from at least about nucleic acid residue 530 to at least about nucleic acid residue 9859 of the recombinant or chimeric HIV-1 nucleic acids described above.

Other aspects of the invention include isolated, chimeric or recombinant nucleic acid comprising a polynucleotide sequence comprising the gag-pro-reverse transcriptase region of the recombinant or chimeric HIV-1 nucleic acids described above. Accordingly, the invention includes isolated, chimeric or recombinant nucleic acid comprising a polynucleotide sequence from at least about nucleic acid residue 785 or at least about nucleic acid residue 863 to at least about nucleic acid residue 4305 of the recombinant or chimeric HIV-1 nucleic acids described above. The invention further provides an HIV-1 virus, e.g., DH12, wherein the gag-pro-reverse transcriptase genes are replaced by the corresponding genes from the recombinant or chimeric HIV-1 nucleic acids of the invention.

Another aspect of the invention includes isolated, chimeric or recombinant nucleic acid comprising a polynucleotide sequence comprising the gag region of the recombinant or chimeric HIV-1 nucleic acids described above. Accordingly, the invention includes isolated, chimeric or recombinant nucleic acid comprising a polynucleotide sequence from at least about nucleic acid residue 863 to at least about nucleic acid residue 2365 of the recombinant or chimeric HIV-1 nucleic acids described above. The invention further provides an HIV-1 virus, e.g., DH12, wherein the gag gene is replaced by the corresponding gene from the recombinant or chimeric HIV-1 nucleic acids of the invention.

Other aspects of the invention include an isolated, chimeric or recombinant nucleic acid comprising a polynucleotide sequence comprising the pro-reverse transcriptase region of the recombinant or chimeric HIV-1 nucleic acids described above. Accordingly, the invention includes isolated, chimeric or recombinant nucleic acid comprising a polynucleotide sequence from at least about nucleic acid residue 2158 to at least about nucleic acid residue 4305 of the recombinant or chimeric HIV-1 nucleic acids described above. The invention further provides an HIV-1 virus, e.g., DH12, wherein the pro-reverse transcriptase genes are replaced by the corresponding genes from the recombinant or chimeric HIV-1 nucleic acids of the invention.

Chimeric or Recombinant or Chimeric HIV-1 Nucleic Acids

In another aspect, the invention includes a chimeric or recombinant or chimeric HIV-1 nucleic acid comprising known DH12 nucleic acid in which one or more recombinant or modified (e.g., shuffled) nucleotide sequences (e.g., a gag, pro, or RT nucleotide sequence, etc.) of a recombinant or chimeric HIV-1 nucleic acid of the invention (e.g., of clone 1.4) are substituted for the corresponding respective nucleotide sequences of DH12. One of ordinary skill in the art can readily determine which nucleotide region(s) or segment(s) or sequence(s) of a recombinant or chimeric HIV-1 nucleic acid comprise the gene or genes (e.g., a gag gene, a pro gene, an RT gene, etc.) in question. One can align the polynucleotide sequence of a known HIV-1 virus nucleic acid that comprises, e.g., a gag gene, a pro gene, an RT gene, etc., with the polynucleotide sequences of a recombinant or chimeric HIV-1 nucleic acid of the invention. By comparison (or via functional assays), one of skill can determine which regions of each recombinant or chimeric HIV-1 polynucleotide sequences, e.g., each of SEQ ID NOS:1-7, comprise the gene or genes (e.g., a gag gene, a pro gene, an RT gene, etc.) in question.

The invention relates to chimeric or recombinant nucleic acids that encode at least one modified, recombinant, or chimeric HIV-1 virus that can replicate in macaque monkey cells, the nucleic acids comprising an HIV-1 (e.g., a wild-type HIV-1, e.g., DH-1) nucleic acid having various genes replaced with the corresponding genes from a recombinant or chimeric HIV-1 nucleic acid of the invention.

In one embodiment, the HIV-1 nef gene of the chimeric, recombinant nucleic acids of the invention is replaced with a modified SIV nef gene that comprises the amino acid residue changes R17Y and Q18E. In an alternate embodiment, each thymine is replaced by a uracil in the polynucleotide sequence of the chimeric or recombinant nucleic acid.

In particular embodiments, the modified, recombinant, or chimeric HIV-1 virus encoded by the chimeric or recombinant nucleic acid exhibits enhanced replication in macaque monkey cells, most preferably pig-tailed macaque monkey cells. The invention also provides polypeptides encoded by the chimeric or recombinant nucleic acids, and a recombinant or chimeric HIV-1 virus comprising the chimeric or recombinant nucleic acid.

Accordingly, the invention provides a chimeric or recombinant nucleic acid that encodes at least one modified, recombinant, or chimeric HIV-1 virus that can replicate in macaque monkey cells, the nucleic acid comprising an HIV-1 (e.g., a wild-type HIV-1, e.g., DH-1) nucleic acid having the gag, pro, and reverse transcriptase genes replaced with the corresponding genes from a recombinant or chimeric HIV-1 nucleic acid of the invention. In one embodiment, the recombinant or chimeric HIV-1 nucleic acid of the invention includes a polynucleotide sequence selected from the group of SEQ ID NOS:1-7. In another embodiment, the corresponding genes comprise from at least about nucleic acid residue 785 to at least about nucleic acid residue 4305 of a polynucleotide sequence selected from the group of SEQ ID NOS:1-7. In a still further embodiment, the corresponding genes comprise from at least about nucleic acid residue 785 to at least about nucleic acid residue 4305 of SEQ ID NO:1.

The invention also provides a chimeric or recombinant nucleic acid that encodes at least one modified, recombinant, or chimeric HIV-1 virus that can replicate in macaque monkey cells, the nucleic acid comprising an HIV-1 (e.g., a wild-type HIV-1, e.g., DH-1) nucleic acid having the int, vif, vpr, rev, tat, vpu, and env genes replaced with the corresponding genes from a recombinant or chimeric HIV-1 nucleic acid of the invention. In one embodiment, the recombinant or chimeric HIV-1 nucleic acid of the invention includes a polynucleotide sequence selected from the group of SEQ ID NOS:1-7. In another embodiment, the corresponding genes comprise from at least about nucleic acid residue 4306 to at least about nucleic acid residue 8547 of a polynucleotide sequence selected from the group of SEQ ID NOS:1-7. In a still further embodiment, the corresponding genes comprise from at least about nucleic acid residue 4306 to at least about nucleic acid residue 8547 of SEQ ID NO:1.

The invention further provides a chimeric or recombinant nucleic acid that encodes at least one modified, recombinant, or chimeric HIV-1 virus that can replicate in macaque monkey cells, the nucleic acid comprising an HIV-1 (e.g., a wild-type HIV-1, e.g., DH-1) nucleic acid having nef-LTR (long-term repeat) gene replaced the corresponding genes from a recombinant or chimeric HIV-1 nucleic acid of the invention. In one embodiment, the recombinant or chimeric HIV-1 nucleic acid of the invention comprises a polynucleotide sequence selected from the group of SEQ ID NOS:1-7. In another embodiment, the corresponding genes comprise from at least about nucleic acid residue 8548 to at least about nucleic acid residue 9942 of a polynucleotide sequence selected from the group of SEQ ID NOS:1-7. In a still further embodiment, the corresponding genes comprise from at least about nucleic acid residue 8548 to at least about nucleic acid residue 9942 of SEQ ID NO:1.

Also included in the invention are chimeric or recombinant nucleic acids that encode a modified HIV-1 virus that can replicate in macaque monkey cells, the nucleic acid comprising a recombinant or chimeric HIV-1 nucleic acid of the invention having various genes replaced with corresponding genes from an HIV-1 (e.g., a wild-type HIV-1, e.g., DH-1) virus.

In one embodiment, the HIV-1 nef gene of the chimeric or recombinant nucleic acids of the invention is replaced with a modified SIV nef gene that includes the amino acid residue changes R17Y and Q18E.

In alternate embodiments, these chimeric or recombinant nucleic acids are RNA or DNA nucleic acids. In one embodiment, these chimeric or recombinant nucleic acids are RNA, and each thymine residue is replaced by a uracil residue in the recombinant or chimeric HIV-1 nucleic acid. In preferred embodiments, the recombinant or chimeric HIV-1 nucleic acid comprises from about nucleic acid residue 530 to about nucleic acid residue 9859.

In one embodiment, the modified HIV-1 virus encoded by the chimeric or recombinant nucleic acid exhibits enhanced replication in macaque monkey cells in vivo. In preferred embodiments, the macaque monkey cells are pig-tailed macaque monkey cells.

The invention also provides polypeptides encoded by the chimeric or recombinant nucleic acids of the invention, and modified HIV-1 viruses comprising the chimeric or recombinant nucleic acids of the invention.

Accordingly, the invention provides a chimeric or recombinant nucleic acid that encodes at least one modified, recombinant, or chimeric HIV-1 virus that can replicate in macaque monkey cells, the nucleic acid comprising a recombinant or chimeric HIV-1 nucleic acid of the invention having the gag, pro, and reverse transcriptase genes replaced with the corresponding genes from an HIV-1 (e.g., a wild-type HIV-1, e.g., DH-1) virus. In one embodiment, the recombinant or chimeric HIV-1 nucleic acid comprises a first RNA genome corresponding to a polynucleotide sequence selected from the group of SEQ ID NOS:1-7 in which each thymine is replaced by a uracil. In another embodiment the gag, pro, and reverse transcriptase genes comprise from at least 863 to at least about nucleic acid residue 4305 of the polynucleotide sequence selected from the group of SEQ ID NOS:1-7. In a still further embodiment, the gag, pro, and reverse transcriptase genes comprise from at least about nucleic acid residue 785 to at least about nucleic acid residue 4305 of the polynucleotide sequence selected from the group of SEQ ID NOS:1-7. In a preferred embodiment, the HIV-1 virus comprises DH12 and the corresponding genes comprise at least about nucleic acid residue 710 to at least about nucleic acid residue 4223 of the DH12 polynucleotide sequence. In another embodiment, the recombinant or chimeric HIV-1 nucleic acid comprises a DNA polynucleotide sequence corresponding to nucleic acid residues 530-9859 of a polynucleotide sequence selected from the group of SEQ ID NOS:1-7.

The invention also provides a chimeric or recombinant nucleic acid that encodes at least one modified, recombinant, or chimeric HIV-1 virus that can replicate in macaque monkey cells, the nucleic acid comprising a recombinant or chimeric HIV-1 nucleic acid of the invention having the int, vif, vpr, rev, tat, vpu, and env genes replaced with the corresponding genes from an HIV-1 (e.g., a wild-type HIV-1, e.g., DH-1) virus. In one embodiment, the recombinant or chimeric HIV-1 nucleic acid comprises from about nucleic acid residue 530 to about nucleic acid residue 9859 of a polynucleotide sequence selected from the group of SEQ ID NOS:1-7 in which each thymine is replaced by a uracil. In another embodiment, the int, vif, vpr, rev, tat, vpu, and env genes comprise from at least about nucleic acid residue 4306 to at least about nucleic acid residue 8547 of a polynucleotide sequence from the group of SEQ ID NOS:17 in which each thymine is replaced by a uracil. In a still further embodiment, the HIV-1 virus comprises DH12 and corresponding genes comprise from at least about nucleic acid residue 4224 to at least about nucleic acid residue 8465 of the DH12 polynucleotide sequence, wherein each thymine is replaced by a uracil. In another embodiment, the recombinant or chimeric HIV-1 nucleic acid comprises a DNA polynucleotide sequence corresponding to nucleic acid residues 530-9859 of a polynucleotide sequence selected from the group of SEQ ID NOS:1-7.

The invention also provides a chimeric or recombinant nucleic acid that encodes at least one modified, recombinant, or chimeric HIV-1 virus that can replicate in macaque monkey cells, the nucleic acid comprising a recombinant or chimeric HIV-1 nucleic acid of the invention having the nef-LTR gene replaced with the corresponding gene from an HIV-1 (e.g., a wild-type HIV-1, e.g., DH-1) virus. In one embodiment, the recombinant or chimeric HIV-1 nucleic acid comprises from about nucleic acid residue 530 to about nucleic acid residue 9859 of a polynucleotide selected from the group of SEQ ID NOS:1-7 wherein each thymine is replaced by a uracil. In another embodiment, the nef-LTR gene comprises from at least about nucleic acid residue 8548 to at least about nucleic acid residue 9942 of a polynucleotide sequence from the group of SEQ ID NOS:1-7 in which each thymine is replaced by a uracil. In another embodiment, the nef-LTR gene comprises from at least about nucleic acid residue 8548 to at least about nucleic acid residue 9942 of SEQ ID NO:1, in which each thymine is replaced by a uracil. In a still further embodiment, the HIV-1 virus comprises DH12 and corresponding gene comprises from at least about nucleic acid residue 8466 to at least about nucleic acid residue 9704 of the DH12 polynucleotide sequence, wherein each thymine is replaced by a uracil. In another embodiment, the recombinant or chimeric HIV-1 nucleic acid comprises a DNA polynucleotide sequence corresponding to nucleic acid residues 530-9859 of a polynucleotide sequence selected from the group of SEQ ID NOS:1-7.

As described in more detail herein, the nucleic acids of the invention include sequences which encode HIV-1 polypeptides and sequences complementary to the coding sequences, and novel fragments of coding sequences and complements thereof. The nucleic acids of the invention can be in the form of RNA or in the form of DNA, and include mRNA, cRNA, synthetic RNA and DNA, and cDNA. The nucleic acids can be double-stranded or single-stranded, and if single-stranded, can be the coding strand or the non-coding (anti-sense, complementary) strand. The nucleic acids optionally include the coding sequence of an HIV-1 polypeptide (i) in isolation, (ii) in combination with additional coding sequence, so as to encode, e.g., a fusion protein, a pre-protein, a prepro-protein, or the like, (iii) in combination with non-coding sequences, such as introns, control elements such as a promoter, a terminator element, or 5′ and/or 3′ untranslated regions effective for expression of the coding sequence in a suitable host, and/or (iv) in a vector or host environment in which the HIV-1 polypeptide coding sequence is a heterologous nucleic acid sequence or gene. Sequences can also be found in combination with typical compositional formulations of nucleic acids, including in the presence of carriers, buffers, adjuvants, excipients, and the like.

The term DNA or RNA includes any oligodeoxyribonucleotide or oligoribonucleotide sequence that, upon expression in an appropriate host cell, results in production of an HIV-1 polypeptide of the invention. The DNA or RNA can be produced in an appropriate host cell or can be produced synthetically (e.g., by an amplification technique such as PCR) or chemically.

Variants of Recombinant or Chimeric HIV-1 Nucleic Acids

The invention provides each and every possible variation of polynucleotide sequence encoding a polypeptide of the invention that could be made by selecting combinations based on possible codon choices. These combinations are made in accordance with the standard triplet genetic code as applied to the polynucleotide sequence encoding recombinant or chimeric HIV-1 polypeptides of the invention. All such variations of every nucleic acid herein are specifically provided and described by consideration of the sequence in combination with the genetic code. It will be appreciated by those skilled in the art that due to the degeneracy of the genetic code, a multitude of nucleic acids encoding recombinant or chimeric HIV-1 polypeptides of the invention can be produced, some which may bear minimal sequence homology to the recombinant or chimeric HIV-1 polynucleotide sequences explicitly disclosed herein. Also contemplated by the present invention are those nucleic acids that, due to the inherent degeneracy of the genetic code, encode substantially the same or a functionally equivalent recombinant or chimeric HIV-1 polypeptides of the invention.

Additionally, the present invention includes a nucleic acid that encodes a recombinant or chimeric HIV-1 polypeptide having an amino acid sequence of SEQ ID NO:8 to SEQ ID NO:39 that contains one or more conservatively modified variations, e.g., one or more substitutions, additions, or deletions that alter, add, or delete a single amino acid or a small percentage of amino acids (typically less than about 5%, more typically less than about 4%, 2%, or 1%), as described in greater detail below.

The term “conservatively modified variations” or, simply, “conservative variations” or “conservative substitutions” of a particular nucleic acid sequence refers to those nucleic acids which encode identical or essentially identical amino acid sequences, or, where the nucleic acid does not encode an amino acid sequence, to essentially identical sequences. One of ordinary skill in the art will recognize that individual substitutions, deletions or additions which alter, add or delete a single amino acid or a small percentage of amino acids (typically less than 5%, more typically less than 4%, 2% or 1%, or less) in an encoded sequence are “conservatively modified variations” where the alterations result in the deletion of an amino acid, addition of an amino acid, or substitution of an amino acid with a chemically similar amino acid.

Conservative substitution tables providing functionally similar amino acids are well known those of ordinary skill in the art. For example, amino acids can be grouped by similar function or chemical structure or composition (e.g., acidic, basic, aliphatic, aromatic, sulfur-containing). See also Creighton (1984) Proteins, W.H. Freeman and Company, for additional groupings of amino acids.

Thus, “conservatively substituted variations” of a listed polypeptide sequence of the present invention include substitutions of a small percentage, typically less than 5%, more typically less than 2% or 1%, or less, of the amino acids of the polypeptide sequence, with a conservatively selected amino acid of the same conservative substitution group. Listing of a protein sequence herein, in conjunction with the above substitution table, provides an express listing of all conservatively substituted proteins.

The addition of one or more nucleic acids or sequences that do not alter the encoded activity of a nucleic acid molecule of the invention, such as the addition of a nonfunctional sequence, is a conservative variation of the basic nucleic acid molecule, and the addition of one or more amino acid residues that do not alter the activity of a polypeptide of the invention is a conservative variation of the basic polypeptide. Both such types of additions are features of the invention.

One of ordinary skill in the art will appreciate that many conservative variations of the nucleic acid constructs that are disclosed yield a functionally identical construct. For example, as discussed above, owing to the degeneracy of the genetic code, “silent substitutions” (i.e., substitutions in a nucleic acid sequence which do not result in an alteration in an encoded polypeptide) are an implied feature of every nucleic acid sequence that encodes an amino acid. Similarly, “conservative amino acid substitutions,” in one or a few amino acids in an amino acid sequence are substituted with different amino acids with highly similar properties, are also readily identified as being highly similar to a disclosed construct. Such conservative variations of each disclosed sequence are a feature of the present invention.

Target nucleic acids which hybridize to the polynucleotide sequences represented by SEQ ID NOS:1-7, or nucleic acids encoding the polypeptides of SEQ ID NOS:8-39, or complementary sequences thereof, including fragments and subsequences, under stringent conditions and high, ultra-high and ultra-ultra high stringency conditions are a feature of the invention. Examples of such nucleic acids include those with one or a few silent or conservative nucleic acid substitutions as compared to a given nucleic acid sequence.

Detection of stringent hybridization in the context of the present invention indicates relatively strong structural similarity/homology to, e.g., the nucleic acids provided in the sequence listings herein. Detection of highly stringent hybridization between two polynucleotide sequences demonstrates a degree of similarity or homology of structure, nucleotide base composition, arrangement or order that is greater than that detected by stringent hybridization conditions.

Comparative hybridization can be used to identify target nucleic acids of the invention, and this comparative hybridization method is a preferred method of distinguishing nucleic acids of the invention. For example, it is desirable to identify target nucleic acids that hybridize to the exemplar nucleic acids herein under stringent conditions. Stringent (including, e.g. highly stringent, ultra highly stringent, and ultra-ultra highly stringent) hybridization and wash conditions can be determined empirically for any target nucleic acid, as described above.

A target nucleic acid is said to specifically hybridize to a probe nucleic acid when it hybridizes at least ½ as well to the probe as to the perfectly matched complementary target, i.e., with a signal to noise ratio at least ½ as high as hybridization of the probe to the target under conditions in which the perfectly matched probe binds to the perfectly matched complementary target with a signal to noise ratio that is at least about 2.5×-10×, typically 5×-10× as high as that observed for hybridization to any of the unmatched target nucleic acids.

Ultra high-stringency hybridization and wash conditions are those in which the stringency of hybridization and wash conditions are increased until the signal to noise ratio for binding of the probe to the perfectly matched complementary target nucleic acid is at least 10× as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or 500× or more as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-ultra-high stringency conditions.

For the purposes of this invention, the unmatched target nucleic acid is one corresponding to e.g. a known HIV-1 nucleic acid sequence, e.g., an HIV-1 sequence that is present in a public database such as GenBank at the time of filing of the subject application. Examples of such unmatched target nucleic acids include, e.g., nucleic acid sequences represented by GenBank accession Nos.: M19921; AF069140; K03455, M38432; K02013; AF004394; M93258; M38429; M22639; K03454, X04414; X04415, K03456 or by other similar molecules found in any public database. Additional such sequences can be identified in GenBank by one of ordinary skill in the art.

Target nucleic acids that do not hybridize to each other under stringent conditions are still substantially identical if the polypeptides that they encode are substantially identical; these target nucleic acids are also a feature of the invention. This occurs, e.g., when a copy of a nucleic acid is created using the maximum codon degeneracy permitted by the genetic code, or when antiserum or antisera generated against one or more of SEQ ID NOS:8-39 which has been subtracted using the polypeptides encoded by known HIV-1 polynucleotide sequences, including, e.g., the following HIV-1 sequences in GenBank: M19921; AF069140; K03455, M38432; K02013; AF004394; M93258; M38429; M22639; K03454, X04414; X04415, K03456 or other similar HIV-1 sequences presented in GenBank. Further details on immunological identification of polypeptides of the invention are found below.

Nucleic Acid Compositions

The invention also contemplates standard manipulations of the nucleic acids of the invention and therefore includes compositions that represent the intermediates or end-products of standard recombinant DNA techniques. Thus, for example, the invention includes a composition produced by the cleaving of one or more the nucleic acids, e.g., by mechanical, chemical, or enzymatic means. Examples of enzymes suitable for enzymatic cleavage include a restriction endonuclease, an RNAse or a DNAse, and the like. The invention also includes a composition produced by a process comprising incubating one or more of the nucleic acids in the presence of deoxyribonucleotide triphosphates and a nucleic acid polymerase.

In an exemplary embodiment, the nucleic acid polymerase is a thermostable polymerase, such as those useful in amplification methods. Examples of in vitro amplification methods, including the polymerase chain reaction (PCR) the ligase chain reaction (LCR), Qβ-replicase amplification and other RNA polymerase mediated techniques are found in Berger, Sambrook, and Ausubel, as well as Mullis et al. (1987) U.S. Pat. No. 4,683,202; PCR Protocols A Guide to Methods and Applications (Innis et al. eds.) Academic Press Inc., San Diego, Calif. (1990) (“Innis”); Arrheim & Levinson (Oct. 1, 1990) C&EN 36-47; The Journal Of NIH Research (1991) 3:81-94; Kwoh et al. (1989) Proc. Natl. Acad. Sci. USA 86:1173; Guatelli et al. (1990) Proc. Natl Acad. Sci. USA 87:1874; Lomell et al. (1989) J. Clin. Chem. 35:1826; Landegren et al. (1988) Science 241:1077-1080; Van Brunt (1990) Biotechnology 8:291-294; Wu and Wallace (1989) Gene 4:560; and Barringer et al. (1990) Gene 89:117. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369:684-685 and the references therein, in which PCR amplicons of up to 40 kb are generated.

One of ordinary skill in the art will appreciate that essentially any RNA can be converted into a double stranded DNA suitable for restriction digestion, PCR expansion and sequencing using reverse transcriptase and a polymerase. See Ausubel, Sambrook and Berger, all supra.

Vectors, Promoters, and Expression Systems

The present invention also includes vectors comprising one or more of the polynucleotide sequences as broadly described above (e.g., recombinant or chimeric HIV-1 nucleic acids of the invention or nucleic acids encoding a recombinant or chimeric HIV-1 polypeptide of the invention or a nucleic acid fragment thereof). In some embodiments, the vector comprises a plasmid, a cosmid, or a phage, or encodes a virus or virus-like particle (VLP). In another embodiment, the vector comprises an expression vector. The invention also provides one or more cells or a population of cells including these vectors. Also provided are one or more cells or population of cells that comprise the recombinant or chimeric HIV-1 nucleic acids of the invention. In a preferred embodiment, such cell or cells express at least one polypeptide encoded by the nucleic acid.

The constructs comprise a vector, such as a plasmid, a cosmid, a phage, a virus (including a retrovirus), a bacterial artificial chromosome (BAC), a yeast artificial chromosome (YAC), and the like, into which a polynucleotide sequence of the invention has been inserted, in a forward or reverse orientation. General texts that describe molecular biological techniques useful herein, including the use of vectors, promoters and many other relevant topics, include Sambrook, Ausubel, and Berger, supra.

In an aspect of this embodiment, the construct further comprises regulatory sequences, including, for example, a promoter, operably linked to the sequence. Large numbers of suitable vectors and promoters are known to those of ordinary skill in the art, and are commercially available. Mutated, recombinant, or recursively recombined (e.g., shuffled) promoters, including those derived from HIV-1 promoters, can be used with polynucleotide sequences of the invention.

The nucleic acids of the present invention can be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others. Any vector that transduces genetic material into a cell, and, if replication is desired, that is replicable and viable in the relevant host can be used. The nucleic acid sequence in the expression vector is operatively linked to an appropriate transcription control sequence (promoter) to direct mRNA synthesis. Examples of such promoters include: LTR or SV40 promoter, E. coli lac or trp promoter, phage lambda PL promoter, and other promoters known to control expression of genes in prokaryotic or eukaryotic cells or their viruses. The expression vector also contains a ribosome binding site for translation initiation, and a transcription terminator. The vector optionally comprises appropriate sequences for amplifying expression. In addition, the expression vectors optionally comprise one or more selectable marker genes to provide a phenotypic trait for selection of transformed host cells, such as dihydrofolate reductase or neomycin resistance for eukaryotic cell culture, or such as tetracycline or ampicillin resistance in E. coli.

Host cells are genetically engineered (i.e., transduced, transformed or transfected) with the vectors of this invention, which can be, for example, a cloning vector or an expression vector. The vector can be, for example, in the form of a plasmid, a viral particle, a phage, etc. The engineered host cells can be cultured in conventional nutrient media modified as appropriate for activating promoters, selecting transformants, or amplifying the relevant HIV-1 gene. The culture conditions, such as temperature, pH and the like, are those previously used with the host cell selected for expression, and will be apparent to those skilled in the art and in the references cited herein.

The vector containing the appropriate DNA sequence as described above, as well as an appropriate promoter or control sequence, can be employed to transform an appropriate host to permit the host to express the protein. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as 293T, CHO, COS, BHK, HEK or Bowes melanoma; plant cells, etc. It is understood that not all cells or cell lines need to be capable of producing fully functional HIV-1 proteins. The invention is not limited by the host cells employed.

In bacterial systems, a number of expression vectors can be selected depending upon the use intended for the recombinant or chimeric HIV-1 polypeptides. For example, when large quantities of HIV-1 polypeptides or fragments thereof are needed for the induction of antibodies, vectors that direct high level expression of fusion proteins that are readily purified can be desirable. Such vectors include, but are not limited to, multifunctional E. coli cloning and expression vectors such as BLUESCRIPT (Stratagene), in which the HIV-1 polypeptide coding sequence can be ligated into the vector in-frame with sequences for the amino-terminal Met and the subsequent 7 residues of beta-galactosidase so that a hybrid protein is produced; pIN vectors (Van Heeke & Schuster (1989) J. Biol. Chem. 264:5503-5509); pET vectors (Novagen, Madison, Wis.); and the like.

Similarly, in the yeast Saccharomyces cerevisiae a number of vectors containing constitutive or inducible promoters such as alpha factor, alcohol oxidase and PGH can be used for production of the HIV-1 polypeptides of the invention. For reviews, see Ausubel (supra) and Grant et al. (1987) Methods in Enzymology 153:516-544.

In mammalian host cells, a number expression systems, such as viral-based systems, can be utilized. In cases where an adenovirus is used as an expression vector, a coding sequence is optionally ligated into an adenovirus transcription/translation complex comprising of the late promoter and tripartite leader sequence. Insertion in a nonessential E1 or E3 region of the viral genome will result in a viable virus capable of expressing the HIV-1 polypeptide in infected host cells (Logan and Shenk (1984) Proc. Nat'l Acad. Sci. 81:3655-3659). In addition, transcription enhancers, such as the rous sarcoma virus (RSV) enhancer, can be used to increase expression in mammalian host cells.

Modified Coding Sequences

Another aspect of the invention are nucleic acids encoding a recombinant or chimeric HIV-1 polypeptide of the invention, wherein the coding sequence has been modified to optimize expression. As will be understood by those of ordinary skill in the art, it can be advantageous to modify a coding sequence (including, e.g., a polynucleotide sequence encoding an recombinant or chimeric HIV-1 polypeptide of the invention or a fragment thereof) to enhance its expression in a particular host. The genetic code is redundant with 64 possible codons, but most organisms preferentially use a subset of these codons. The codons that are utilized most often in a species are called optimal codons, and those not utilized very often are classified as rare or low-usage codons (see, e.g., Zhang, S. P. et al. (1991) Gene 105:61-72). Codons can be substituted to reflect the preferred codon usage of the host, a process called “codon optimization” or “controlling for species codon bias.”

Optimized coding sequence containing codons preferred by a particular prokaryotic or eukaryotic host (see also Murray, E. et al. (1989) Nuc. Acids. Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, preferred stop codons for S. cerevisiae and mammals are UAA and UGA respectively. The preferred stop codon for monocotyledonous plants is UGA, whereas insects and E. coli prefer to use UAA as the stop codon (Dalphin, M. E. et al. (1996) Nuc. Acids. Res. 24:216-218). The polynucleotide sequences of the present invention can be engineered in order to alter a recombinant or chimeric HIV-1 polypeptide coding sequence for a variety of reasons, including but not limited to, alterations which modify the cloning, processing and/or expression of the gene product. For example, alterations can be introduced using techniques which are well known in the art, e.g., site-directed mutagenesis, to insert new restriction sites, to alter glycosylation patterns, to change codon preference, to introduce splice sites, etc.

Additional Expression Elements

Specific initiation signals can aid in efficient translation of a recombinant or chimeric HIV-1 polypeptide coding sequence. These signals can include, e.g., the ATG initiation codon and adjacent sequences. In cases where a recombinant or chimeric HIV-1 polypeptide coding sequence, its initiation codon and upstream sequences are inserted into the appropriate expression vector, no additional translational control signals may be needed. However, in cases where only coding sequence (e.g., a mature protein coding sequence), or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression can be enhanced by the inclusion of enhancers appropriate to the cell system in use (Scharf, D. et al. (1994) Results Probl. Cell Differ. 20:125-62; Bittner et al. (1987) Results Probl. Cell Differ. 153:516-544).

Polynucleotides of the invention can also be fused, for example, in-frame to nucleic acid encoding a secretion/localization sequence, to target polypeptide expression to a desired cellular compartment, membrane, or organelle, or to direct polypeptide secretion to the periplasmic space or into the cell culture media. Such sequences are known to those of ordinary skill, and include secretion leader peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like.

The polynucleotides of the present invention can also comprise a coding sequence fused in-frame to a marker sequence that, e.g., facilitates purification of the encoded polypeptide. Such purification facilitating domains include, but are not limited to, metal chelating peptides such as histidine-tryptophan modules that allow purification on immobilized metals, a sequence (e.g., GST) which binds glutathione, a hemagglutinin (HA) tag (corresponding to an epitope derived from the influenza hemagglutinin protein; Wilson et al. (1984) Cell 37:767), maltose binding protein sequences, the FLAG epitope utilized in the FLAGS extension/affinity purification system (Immunex Corp, Seattle, Wash.), and the like. The inclusion of a protease-cleavable polypeptide linker sequence between the purification domain and the HIV-1 polypeptide sequence is useful to facilitate purification.

One expression vector contemplated for use in the compositions and methods described herein provides for expression of a fusion protein comprising a polypeptide of the invention fused to a polyhistidine region separated by an enterokinase cleavage site. The histidine residues facilitate purification on IMIAC (immobilized metal ion affinity chromatography, as described in Porath et al. (1992) Protein Expression and Purification 3:263-281) while the enterokinase cleavage site provides a means for separating the HIV-1 polypeptide from the fusion protein. Vectors such as pGEX (Promega; Madison, Wis.) can also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Expression Hosts

In a further embodiment, the present invention relates to host cells containing the above-described constructs. The host cell can be a eukaryotic cell, such as a mammalian cell, a yeast cell, or a plant cell, or the host cell can be a prokaryotic cell, such as a bacterial cell. Introduction of the construct into the host cell can be effected by calcium phosphate transfection, DEAE-Dextran mediated transfection, electroporation, or other common techniques (Davis, L., Dibner, M., and Battey, I. (1986) Basic Methods in Molecular Biology).

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing that cleaves a “pre” or a “prepro” form of the protein may also be important for correct insertion, folding and/or function. Different host cells such as 293T, CHO, HeLa, BHK, MDCK, WI38, etc. have specific cellular machinery and characteristic mechanisms for such post-translational activities and can be chosen to ensure the correct modification and processing of the introduced, foreign protein.

For long-term, high-yield production of recombinant proteins, stable expression can be used. For example, cell lines that stably express a polypeptide of the invention are transduced using expression vectors which contain viral origins of replication or endogenous expression elements and a selectable marker gene. Following the introduction of the vector, cells can be allowed to grow for 1-2 days in an enriched media before they are switched to selective media. The purpose of the selectable marker is to confer resistance to selection, and its presence allows growth and recovery of cells that successfully express the introduced sequences. For example, resistant clumps of stably transformed cells can be proliferated using tissue culture techniques appropriate to the cell type.

Host cells transformed with a nucleotide sequence encoding a polypeptide of the invention are optionally cultured under conditions suitable for the expression and recovery of the encoded protein from cell culture. The protein or fragment thereof produced by a recombinant cell can be secreted, membrane-bound, or contained intracellularly, depending on the sequence and/or the vector used. As will be understood by those of ordinary skill in the art, expression vectors containing polynucleotides encoding the retrovirus HIV-1 polypeptides of the invention can be designed with signal sequences that direct secretion of the mature polypeptides through a prokaryotic or eukaryotic cell membrane.

Making Nucleic Acids of the Invention

Nucleic acids of the invention can be prepared by standard solid-phase methods, according to known synthetic methods. Typically, fragments of up to about 20, 30, 40, 50, 60, 70, 80, 90, and/or 100 bases are individually synthesized, then joined (e.g., by enzymatic or chemical ligation methods, or polymerase mediated recombination methods) to form essentially any desired continuous sequence. In another aspect, fragments of greater than 100 bases (e.g., 150, 200, 300, 400, 500, 550, 600, 650 bases) are individually synthesized, then joined (e.g., by enzymatic or chemical ligation methods, or polymerase mediated recombination methods) to form essentially any desired continuous sequence.

For example, the nucleic acids of the invention, including fragments thereof (and those as described above), can be prepared by chemical synthesis using, e.g., the classical phosphoramidite method described by Beaucage et al. (1981) Tetrahedron Letters 22:1859-69, or the method described by Matthes et al. (1984) EMBO J. 3:801-05., e.g., as is typically practiced in automated synthetic methods. According to the phosphoramidite method, oligonucleotides are synthesized, e.g., in an automatic DNA synthesizer, purified, annealed, ligated and cloned in appropriate vectors.

In addition, essentially any nucleic acid can be custom ordered from any of a variety of commercial sources, such as The Midland Certified Reagent Company (mcrc@oligos.com), The Great American Gene Company (http://www.genco.com), ExpressGen Inc. (www.expressgen.com), Operon Technologies Inc. (Alameda, Calif.) and many others. Similarly, peptides and antibodies can be custom ordered from any of a variety of sources, such as PeptidoGenic (pkim@ccnet.com), HTI Bio-Products, Inc. (http://www.htibio.com), BMA Biomedicals Ltd (U.K.), Bio.Synthesis, Inc., and many others.

Certain nucleic acids of the invention can also be obtained by screening cDNA libraries (e.g., libraries generated by recursive sequence recombination of nucleic acid segments or other diversity generation methods (e.g., DNA shuffling)) using oligonucleotide probes which can hybridize to or PCR-amplify nucleic acids of the invention. Procedures for screening and isolating cDNA clones are well-known to those of ordinary skill in the art. Such techniques are described in, for example, Sambrook and Ausubel, supra.

Recombinant or Chimeric HIV-1 Polypeptides

The invention provides recombinant or chimeric HIV-1 polypeptides, fragments of recombinant or chimeric HIV-1 polypeptides, related fusion recombinant or chimeric HIV-1 polypeptides, or functional equivalents thereof. A recombinant or chimeric HIV-1 polypeptide of the invention includes a polypeptide comprising an amino acid sequence selected from SEQ ID NOS:8-39, and variations thereof, e.g., conservatively modified variations thereof.

The invention provides an isolated, chimeric or recombinant polypeptide comprising an amino acid sequence selected from the group of: (a) an amino acid sequence that has at least about 95% sequence identity to at least one sequence from the group of SEQ ID NOS:8, 17, 22, 27, and 34; (b) an amino acid sequence that has at least about 95% sequence identity to at least one amino acid sequence of the group of SEQ ID NOS:9, 20, 23, and 35; (c) SEQ ID NOS:10, 18, 28, and 31; (d) SEQ ID NOS:11 and 36; (e) SEQ ID NO:12; (f) SEQ ID NOS:13 and 24; (g) SEQ ID NOS:14 and 37; (h) SEQ ID NOS:15, 25, 29, 32, and 38; and (i) SEQ ID NOS:16, 19, 21, 26, 30, 33, and 39.

The invention also includes polypeptide fragments of the recombinant or chimeric HIV-1 polypeptides of the present invention. In one aspect, recombinant or chimeric HIV-1 viruses comprising said polypeptide fragments exhibit enhanced replication as described herein. Polypeptide fragments can also be made using the techniques described herein.

Gag-Pol Fusion Proteins

The invention also provides fusion proteins comprising fusions of the recombinant gag and pol polypeptides described herein. In one aspect, a gag-pol fusion protein comprises a fusion protein comprising from about amino acid residue 1 to about amino acid residue 432 of a recombinant gag polypeptide described herein (e.g., any of SEQ ID NOS:8, 17, 22, 27, or 34) and about amino acid residue 1 to about amino acid residue 1003 of a recombinant pol polypeptide (e.g., any of SEQ ID NOS:9, 20, 23, and 35) described herein.

Modified Amino Acids

Polypeptides of the invention can contain one or more modified amino acids. The presence of modified amino acids can be advantageous in, for example, (a) increasing polypeptide serum half-life, (b) reducing polypeptide antigenicity, and/or (c) increasing polypeptide storage stability. Amino acid(s) are modified, for example, co-translationally or post-translationally during recombinant production (e.g., N-linked glycosylation at N-X-S/T motifs during expression in mammalian cells) or modified by synthetic means.

Non-limiting examples of a modified amino acid include a glycosylated amino acid, a sulfated amino acid, a prenlyated (e.g., farnesylated, geranylgeranylated) amino acid, an acetylated amino acid, an acylated amino acid, a PEG-ylated amino acid, a biotinylated amino acid, a carboxylated amino acid, a phosphorylated amino acid, and the like. References adequate to guide one of ordinary skill in the art in the modification of amino acids are replete throughout the literature. Example protocols are found in Walker (1998) Protein Protocols on CD-ROM Human Press, Towata, N.J.

Defining Polypeptides by Immunoreactivity

Because the recombinant polypeptides of the invention provide a variety of new polypeptide sequences as compared to other HIV-1 polypeptides, the polypeptides also provide a new structural features that can be recognized, e.g., in immunological assays. The generation of antiserum or antisera that specifically binds the polypeptides of the invention, and the polypeptides which are bound by such antiserum or antisera, are features of the present invention.

The phrase “specifically (or selectively) binds,” “specifically (or selectively bound,” or “specifically (or selectively) immunoreactive with,” when referring to a polypeptide, refers to a binding reaction with an antibody which is determinative of the presence of the polypeptide, or an epitope from the polypeptide, in the presence of a heterogeneous population of polypeptides and other biologics. Specific binding between an antibody or other binding agent and an antigen generally means a binding affinity of at least about 10⁵ to 10⁶ M⁻¹.

Thus, under designated immunoassay conditions, the specified antibodies bind to a particular polypeptide and do not bind in a significant amount to other polypeptides present in the sample. The antibodies raised against a multivalent antigenic polypeptide will generally bind to the polypeptides from which one or more of the epitopes were obtained. Specific binding to an antibody under such conditions may require an antibody that is selected for its specificity for a particular polypeptide. A variety of immunoassay formats can be used to select antibodies specifically immunoreactive with a particular polypeptide. For example, solid-phase ELISA immunoassays, Western blots, or immunohistochemistry are routinely used to select monoclonal antibodies specifically immunoreactive with a protein. See Harlow and Lane (1988) Antibodies, A Laboratory Manual, Cold Spring Harbor Publications, New York (hereinafter “Harlow and Lane”), for a description of immunoassay formats and conditions that can be used to determine specific immunoreactivity. Typically, a specific or selective reaction is at least twice background signal or noise and more typically 2.5×-5× or more than 10 to 100 times background.

The polypeptides of the invention provide structural features that can be recognized, e.g., in immunological assays. The generation of antisera containing antibodies (for at least one antigen) that specifically bind the polypeptides of the invention, as well as the polypeptides that are bound by such antisera, are a feature of the invention. Preferred binding agents, including antibodies described herein, bind polypeptides of the invention and fragments thereof with affinities of at least about 10⁶ to 10⁷ M⁻¹, and preferably 10⁸ M⁻¹ to 10⁹ M⁻¹ or 10¹⁰ M⁻¹. Conventional hybridoma technology can be used to produce antibodies having affinities of up to about 10⁹ M−1. However, new technologies, including phage display and transgenic mice, can be used to achieve higher affinities (e.g., up to at least about 10¹² M⁻¹). In general, a higher binding affinity is advantageous.

The invention includes HIV-1 polypeptides that bind or specifically bind to or that are immunoreactive or specifically immunoreactive with an antibody or antisera (or antiserum) generated against an immunogen comprising an amino acid sequence selected from one or more of SEQ ID NO:8 to SEQ ID NO:14. To eliminate cross-reactivity with other retrovirus HIV-1 polypeptides, e.g., known HIV-1 polypeptides, the antibody or antisera (or antiserum) is subtracted with available known HIV-1 sequences, such as those represented at GenBank accession numbers AAB34096; AAB34095; AAB34094; S80869; S77017; S77015; S77012; J01998, AF169256; and AH000833 (the control HIV-1 polypeptides) and those GenBank accession numbers or GENSEQ database numbers identified above. Where the accession number or database number corresponds to a nucleic acid, a polypeptide encoded by the nucleic acid is generated and used for antibody/antisera (antiserum) subtraction purposes. Where the nucleic acid corresponds to a non-coding sequence, e.g., a pseudo gene, an amino acid that corresponds to the reading frame of the nucleic acid is generated (e.g. synthetically), or is minimally modified to include a start codon for recombinant production.

In one typical format, the immunoassay uses a polyclonal antisera or antiserum which was raised against one or more polypeptides comprising one or more of the sequences corresponding to one or more of: SEQ ID NO:8 to SEQ ID NO:39, or a substantial subsequence or fragment thereof (i.e., at least about 30%, 40%, 50%, 60%, 70%, 80%, 90%, 95%, or 99% of the full length sequence provided). The full set of potential polypeptide immunogens derived from SEQ ID NO:8 to SEQ ID NO:39 are collectively referred to below as “the immunogenic polypeptides.” The resulting antisera or antiserum is optionally selected to have low cross-reactivity against the control HIV-1 polypeptides, and/or other known retrovirus HIV-1 polypeptides, and any such cross-reactivity is removed by immunoabsorption with one or more of the control HIV-1 polypeptides, prior to use of the polyclonal antisera or antiserum in the immunoassay.

In another aspect, the invention provides an antibody or antisera produced by administering a polypeptide of the invention to a mammal, which antibody specifically binds one or more antigen, the antigen comprising a polypeptide comprising one or more of the amino acid sequences SEQ ID NOS:8-39, which antibody does not specifically bind to a known polypeptide or a polypeptide encoded by one or more of GenBank Nucleotide Accession Numbers above.

Also included is an antibody or antisera which specifically binds a polypeptide comprising a sequence selected from SEQ ID NOS:8-39, wherein the antibody does not specifically bind to a known polypeptide or a polypeptide encoded by one or more of GenBank Nucleotide Accession Nos. set forth above.

In order to produce antisera (or antiserum) for use in an immunoassay, one or more of the immunogenic polypeptides is produced and purified as described herein. For example, recombinant protein can be produced in a mammalian cell line. An inbred strain of mice (used in this assay because results are more reproducible due to the virtual genetic identity of the mice) is immunized with the immunogenic protein(s) in combination with a standard adjuvant, such as Freund's adjuvant, and a standard mouse immunization protocol (see Harlow and Lane (1988) Antibodies, A Laboratory Manual, Cold Spring Harbor Publications, New York, for a standard description of antibody generation, immunoassay formats and conditions that can be used to determine specific immunoreactivity). Alternatively, one or more synthetic or recombinant polypeptides derived from the sequences disclosed herein is conjugated to a carrier protein and used as an immunogen.

Polyclonal sera are collected and titered against the immunogenic polypeptide in an immunoassay, for example, a solid phase immunoassay with one or more of the immunogenic proteins immobilized on a solid support. Polyclonal antisera (or antiserum) with a titer of 106 or greater are selected, pooled and subtracted with the control HIV-1 polypeptide polypeptides to produce subtracted pooled titered polyclonal antisera (or antiserum).

The subtracted pooled titered polyclonal antisera (or antiserum) are tested for cross reactivity against the control HIV-1 polypeptides. Preferably at least two of the immunogenic HIV-1 polypeptides are used in this determination, preferably in conjunction with at least two of the control HIV-1 polypeptides, to identify antibodies that are specifically bound by the immunogenic protein(s).

In this comparative assay, discriminatory binding conditions are determined for the subtracted titered polyclonal antisera (or antiserum) which result in at least about a 5-10 fold higher signal to noise ratio for binding of the titered polyclonal antisera (or antiserum) to the immunogenic HIV-1 polypeptides as compared to binding to the control HIV-1 polypeptides. That is, the stringency of the binding reaction is adjusted by the addition of non-specific competitors such as albumin or non-fat dry milk, or by adjusting salt conditions, temperature, or the like. These binding conditions are used in subsequent assays for determining whether a test polypeptide is specifically bound by the pooled subtracted polyclonal antisera (or antiserum). In particular, test polypeptides which show at least a 2-5× higher signal to noise ratio than the control polypeptides under discriminatory binding conditions, and at least about a ½ signal to noise ratio as compared to the immunogenic polypeptide(s), shares substantial structural similarity with the immunogenic polypeptide as compared to known retrovirus HIV-1 polypeptides, and is, therefore a polypeptide of the invention.

In another example, immunoassays in the competitive binding format are used for detection of a test polypeptide. For example, as noted, cross-reacting antibodies are removed from the pooled antisera (or antiserum) mixture by immunoabsorption with the control HIV-1 polypeptide polypeptides. The immunogenic polypeptide(s) are then immobilized to a solid support that is exposed to the subtracted pooled antisera (or antiserum). Test proteins are added to the assay to compete for binding to the pooled subtracted antisera (or antiserum). The ability of the test protein(s) to compete for binding to the pooled subtracted antisera (or antiserum) as compared to the immobilized protein(s) is compared to the ability of the immunogenic polypeptide(s) added to the assay to compete for binding (the immunogenic polypeptides compete effectively with the immobilized immunogenic polypeptides for binding to the pooled antisera (or antiserum)). The percent cross-reactivity for the test proteins is calculated, using standard calculations.

In a parallel assay, the ability of the control proteins to compete for binding to the pooled subtracted antisera (or antiserum) is determined as compared to the ability of the immunogenic polypeptide(s) to compete for binding to the antisera (or antiserum). Again, the percent cross-reactivity for the control polypeptides is calculated, using standard calculations. Where the percent cross-reactivity is at least 5-10× as high for the test polypeptides, the test polypeptides are said to specifically bind the pooled subtracted antisera (or antiserum).

In general, the immunoabsorbed and pooled antisera (or antiserum) can be used in a competitive binding immunoassay as described herein to compare any test polypeptide to the immunogenic polypeptide(s). In order to make this comparison, the two polypeptides are each assayed at a wide range of concentrations and the amount of each polypeptide required to inhibit 50% of the binding of the subtracted antisera (or antiserum) to the immobilized protein is determined using standard techniques. If the amount of the test polypeptide required is less than twice the amount of the immunogenic polypeptide that is required, then the test polypeptide is said to specifically bind to an antibody generated to the immunogenic protein, provided the amount is at least about 5-10× as high as for a control polypeptide.

As a final determination of specificity, the pooled antisera (or antiserum) is optionally fully immunoabsorbed with the immunogenic polypeptide(s) (rather than the control polypeptides) until little or no binding of the resulting immunogenic polypeptide subtracted pooled antisera (or antiserum) to the immunogenic polypeptide(s) used in the immunoabsorption is detectable. This fully immunoabsorbed antisera (or antiserum) is then tested for reactivity with the test polypeptide. If little or no reactivity is observed (i.e., no more than 2× the signal to noise ratio observed for binding of the fully immunoabsorbed antisera (or antiserum) to the immunogenic polypeptide), then the test polypeptide is specifically bound by the antisera (or antiserum) elicited by the immunogenic protein.

Making Polypeptides of the Invention

Recombinant methods for producing and isolating HIV-1 polypeptide polypeptides of the invention are described above. In addition to recombinant production, the polypeptides can be produced by direct peptide synthesis using solid-phase techniques (cf. Stewart et al. (1969) Solid-Phase Peptide Synthesis, W.H. Freeman Co, San Francisco; Merrifield J. (1963) J. Am. Chem. Soc. 85:2149-2154). Peptide synthesis can be performed using manual techniques or by automation. Automated synthesis can be achieved, for example, using Applied Biosystems 431A Peptide Synthesizer (Perkin Elmer, Foster City, Calif.) in accordance with the instructions provided by the manufacturer. For example, subsequences can be chemically synthesized separately and combined using chemical methods to provide full-length HIV-1 polypeptides.

Additional methods for producing the polypeptides of the invention are included. One such method comprises introducing into a population of cells any nucleic acid of the invention described herein, which is operatively linked to a regulatory sequence effective to produce the encoded polypeptide, culturing the cells in a culture medium to produce the polypeptide, and isolating the polypeptide from the cells or from the culture medium. An amount of nucleic acid sufficient to facilitate uptake by the cells (transfection) and/or expression of the polypeptide is utilized. The culture medium can be any described herein and in the Examples. The nucleic acid is introduced into such cells by any delivery method described herein, including, e.g., injection, gene gun, passive uptake, etc. The nucleic acid can be part of a vector, such as a recombinant expression vector, including a DNA plasmid vector, or any vector described herein. The nucleic acid or vector comprising a nucleic acid of the invention described herein can be prepared and formulated as described herein. Such a nucleic acid or expression vector can be introduced into a population of cells of a mammal in vivo, or selected cells of the mammal (e.g., tumor cells) can be removed from the mammal and the nucleic acid expression vector introduced ex vivo into the population of such cells in an amount sufficient such that uptake and expression of the encoded polypeptide results. Or, a nucleic acid or vector comprising a nucleic acid is produced using cultured cells in vitro. In one aspect, the method of producing a polypeptide comprises introducing into a population of cells a recombinant expression vector comprising any nucleic acid described herein in an amount and formula such that uptake of the vector and expression of the polypeptide will result; administering the expression vector into a mammal by any introduction/delivery format described herein; and isolating the polypeptide from the mammal or from a byproduct of the mammal.

In another aspect, the invention provides for the use of any retrovirus envelope polypeptide or nucleic acid (or vector or cell comprising such nucleic acid) or composition thereof for the manufacture of a medicament, prophylactic, therapeutic, drug, or vaccine, including for any therapeutic or prophylactic application relating to treatment of a disease or disorder as described herein.

Substantially Identical Nucleic Acids and Polypeptides

As noted above, the polypeptides and nucleic acids employed in the subject invention need not be identical, but can be substantially identical (or substantially similar), to the corresponding sequence of a recombinant or chimeric HIV-1 nucleic acid or polypeptide or related molecule. The polypeptides can be subject to various changes, such as insertions, deletions, and substitutions, either conservative or non-conservative, where such changes might provide for certain advantages in their use. The polypeptides of the invention can be modified in a number of ways so long as they comprise a sequence substantially similar or substantially identical (as defined below) to a sequence in a recombinant or chimeric HIV-1 nucleic acid or polypeptide.

Alignment and comparison of relatively short amino acid sequences (less than about 30 residues) is typically straightforward. Comparison of longer sequences can require more sophisticated methods to achieve optimal alignment of two sequences. Optimal alignment of sequences for aligning a comparison window can be conducted by the local homology algorithm of Smith and Waterman (1981) Adv Appl Math 2:482, by the homology alignment algorithm of Needleman and Wunsch (1970) J Mol Biol 48:443, by the search for similarity method of Pearson and Lipman (1988) Proc Natl Acad Sci USA 85:2444, by computerized implementations of these algorithms (GAP, BESTFIT, FASTA and TFASTA in the Wisconsin Genetics Software Package Release 7.0, Genetics Computer Group, 575 Science Dr., Madison, Wis.; and BLAST, see, e.g., Altschul et al., (1977) Nuc Acids Res 25:3389-3402 and Altschul et al., (1990) J Mol Biol 215:403-410), or by inspection, with the best alignment (i.e., resulting in the highest percentage of sequence similarity over the comparison window) generated by the various methods being selected.

As applied to polypeptides, the term substantial identity or substantial similarity means that two peptide sequences, when optimally aligned, such as by the programs BLAST, GAP or BESTFIT using default gap weights (described in detail below) or by visual inspection, share at least about 60 percent, 70 percent, or 80 percent sequence identity or sequence similarity, preferably at least about 90 percent amino acid residue sequence identity or sequence similarity, more preferably at least about 95 percent sequence identity or sequence similarity, or more (including, e.g., about 96, 97, 98, 98.5, 99, or more percent amino acid residue sequence identity or sequence similarity). Similarly, as applied in the context of two nucleic acids, the term substantial identity or substantial similarity means that the two nucleic acid sequences, when optimally aligned, such as by the programs BLAST, GAP or BESTFIT using default gap weights (described in detail below) or by visual inspection, share at least about 60 percent, 70 percent, or 80 percent sequence identity or sequence similarity, preferably at least about 90 percent amino acid residue sequence identity or sequence similarity, more preferably at least about 95 percent sequence identity or sequence similarity, or more (including, e.g., about 96, 97, 98, 98.5, 99, or more percent nucleotide sequence identity or sequence similarity).

In one aspect, the present invention provides recombinant or chimeric HIV-1 homologue nucleic acids having at least about 70%, 75%, 80%, 85%, 86%, 87%, 88%, 90%, 91%, 92%, 93%, 94%, 95%, 95.5%, 96%, 96.5%, 97%, 97.5%, 98%, 98.5%, 99%, 99.5%, or more percent sequence identity or sequence similarity with the nucleic acid sequences of any of SEQ ID NOS:1-7, or complementary nucleotide sequences thereof, or any nucleotide fragments of any such nucleotide sequences, which fragments encode a chimeric or recombinant virus or virus-like particle that replicates in non-human mammals (e.g., pig-tailed macaque monkeys). In another aspect, the present invention provides recombinant or chimeric HIV-1 homologue polypeptides having at least about 70%, 75%, 80%, 85%, 86%, 87%, 88%, 90%, 91%, 92%, 93%, 94%, 95%, 95.5%, 96%, 96.5%, 97%, 97.5%, 98%, 98.5%, 99%, 99.5%, or more percent sequence identity or sequence similarity with the amino acid sequences of any of SEQ ID NOS:8-39, or fragments thereof such that a recombinant or chimeric HIV-1 virus comprising said polypeptides replicates in non-human mammals.

Alternatively, parameters are set such that one or more sequences of the invention are identified by alignment to a query sequence selected from among SEQ ID NO:1 to SEQ ID NO:8, while sequences corresponding to unrelated polypeptides, e.g., those encoded by nucleic acid sequence represented by GenBank accession numbers: M19921; AF069140; K03455, M38432; K02013; AF004394; M93258; M38429; M22639; K03454, X04414; X04415, and K03456 are not identified.

Preferably, residue positions that are not identical differ by conservative amino acid substitutions. Conservative amino acid substitution refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

A preferred example of an algorithm that is suitable for determining percent sequence identity or sequence similarity is the FASTA algorithm, which is described in Pearson, W. R. & Lipman, D. J., (1988) Proc Natl Acad Sci USA 85:2444. See also, W. R. Pearson, (1996) Methods Enzymology 266:227-258. Preferred parameters used in a FASTA alignment of DNA sequences to calculate percent identity or percent similarity are optimized, BL50 Matrix 15: −5, k-tuple=2; joining penalty=40, optimization=28; gap penalty −12, gap length penalty=−2; and width 16.

Other preferred examples of algorithms that are suitable for determining percent sequence identity or sequence similarity are the BLAST and BLAST 2.0 algorithms, which are described in Altschul et al., (1977) Nuc Acids Res 25:3389-3402 and Altschul et al., (1990) J Mol Biol 215:403-410, respectively. BLAST and BLAST 2.0 are used, with the parameters described herein, to determine percent sequence identity or percent sequence similarity for the nucleic acids and polypeptides and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see, Henikoff & Henikoff, (1989) Proc Natl Acad Sci USA 89:10915) uses alignments (B) of 50, expectation (E) of 10, M=5, N-4, and a comparison of both strands. Again, as with other suitable algorithms, the stringency of comparison can be increased until the program identifies only sequences that are more closely related to those in the sequence listings herein (i.e., SEQ ID NO:1 to SEQ ID NO:7 or, alternatively, SEQ ID NO:8 to SEQ ID NO:39), rather than sequences that are more closely related to other similar sequences such as, e.g., those nucleic acid sequences represented by GenBank accession numbers: M19921; AF069140; K03455, M38432; K02013; AF004394; M93258; M38429; M22639; K03454, X04414; X04415, and K03456 or other similar molecules found in, e.g., GenBank. In other words, the stringency of comparison of the algorithms can be increased so that all known prior art (e.g., those represented by GenBank accession numbers: M19921; AF069140; K03455, M38432; K02013; AF004394; M93258; M38429; M22639; K03454, X04414; X04415, and K03456 or other similar molecules found in, e.g., GenBank) is excluded.

The BLAST algorithm also performs a statistical analysis of the similarity or identity between two sequences (see, e.g., Karlin & Altschul, (1993) Proc Natl Acad Sci USA 90:5873-5787). One measure of similarity or identity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity or percent sequence similarity. It also plots a tree or dendogram showing the clustering relationships used to create the alignment. PILEUP uses a simplification of the progressive alignment method of Feng & Doolittle, (1987) J Mol Evol 35:351-360. The method used is similar to the method described by Higgins & Sharp, (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using the following parameters: default gap weight (3.00), default gap length weight (0.10), and weighted end gaps. PILEUP can be obtained from the GCG sequence analysis software package, e.g., version 7.0 (Devereaux et al., (1984) Nuc Acids Res 12:387-395).

Another preferred example of an algorithm that is suitable for multiple DNA and amino acid sequence alignments is the CLUSTALW program (Thompson, J. D. et al., (1994) Nuc Acids Res 22:4673-4680). CLUSTALW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, (1992) Proc Natl Acad Sci USA 89:10915-10919). Another example of an algorithm suitable for multiple DNA and amino acid sequence alignments is the Jotun Hein method, Hein (1990), from within the MegaLine™ DNASTAR package (MegaLine™ Version 4.03, manufactured by DNASTAR, Inc.) used according to the manufacturer's instructions and default values specified in the program.

It will be understood by one of ordinary skill in the art, that the above discussion of search and alignment algorithms also applies to identification and evaluation of polynucleotide sequences, with the substitution of query sequences comprising nucleotide sequences, and where appropriate, selection of nucleic acid databases.

Uses of Recombinant or Chimeric Nucleic Acids of the Invention

The nucleic acids of the invention have a variety of uses in, for example: recombinant production (i.e., expression) of the HIV-1 polypeptides invention; as therapeutics or prophylactics, e.g., for use in gene therapy methods, treatment regimens and related applications, and diagnostic assays; as immunogens; as diagnostic probes for the presence of complementary or partially complementary nucleic acids (including for detection of natural retroviral coding nucleic acids); as substrates for further reactions, e.g., recursive sequence recombination of nucleic acid segments or other diversity generation methods (e.g., DNA shuffling, mutation reactions) to produce new and/or improved HIV-1 polypeptides; and the like. In addition, the nucleic acids of the invention are useful for producing HIV-1 particles that exhibit enhanced replication in non-human animal cells both ex vivo and in vivo.

The recombinant or chimeric HIV-1 nucleic acids and viruses encoded therefrom are useful in producing animal models of HIV-1 infection (e.g., a macaque monkey model); these animal models are useful for the study of HIV-1 pathogenesis and for testing prophylactic and therapeutic strategies and agents for treating and controlling HIV infection. In addition, recombinant or chimeric HIV-1 nucleic acids, viruses encoded therefrom, and polypeptides of the invention, and compositions comprising one or more of these, are useful as attenuated vaccines and subunit vaccines for mammals for, e.g., non-humans and human mammals.

Substrates and Formats for Sequence Recombination

The polynucleotides of the invention are particularly useful in the development and production of HIV-1 viruses with that exhibit enhanced replication in non-human mammalian cells. For example, the recombinant or chimeric HIV-1 nucleic acids are used in any number and/or in any combination as substrates for a variety of recombination and recursive recombination (e.g., DNA shuffling) reactions, as well as other diversity generating techniques, including mutagenesis techniques and standard cloning methods as set forth in, e.g., Ausubel, Berger and Sambrook, i.e., to produce additional recombinant or chimeric HIV-1 polypeptides with desired properties. Based on the screening or selection protocols employed, recombinant, e.g., shuffled, recombinant or chimeric HIV-1 polypeptides can be generated and isolated that confer a variety of desirable characteristics, e.g., enhanced replication in non-human mammalian cells, etc.

Other aspects of the invention relate to a method of producing a further modified or recombinant nucleic acid that entails mutating or recombining the recombinant or chimeric HIV-1 nucleic acid of the invention. In one embodiment, the method entails recursively recombining the recombinant or chimeric HIV-1 nucleic acid of the invention with one or more additional nucleic acids. In alternate embodiments, the mutating or recombining is performed in vitro or in vivo. In a further embodiment, the method entails producing at least one library of further modified or recombinant nucleic acids, said library comprising at least one nucleic acid, wherein at least one modified, recombinant, or chimeric HIV-1 virus comprising said at least one nucleic acid exhibits enhanced replication in non-human mammalian cells. The invention also includes the library produced by this method and a population of cells comprising this library.

The invention also includes the further modified or recombinant nucleic acid produced by this method, wherein at least one modified, recombinant, or chimeric HIV-1 virus comprising said further modified or recombinant nucleic acid exhibits enhanced replication in non-human mammalian cells. In a preferred embodiment, the enhanced replication includes an ability to replicate at a greater rate or for a longer period in vitro in macaque monkey cells or in vivo in a macaque monkey compared to an HIV-1 virus ability to replicate in vitro in macaque monkey cells or in vivo in a macaque monkey.

A variety of diversity generating protocols are available and described in the art. The procedures can be used separately, and/or in combination to produce one or more variants of a nucleic acid or set of nucleic acids, as well variants of encoded polypeptides. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids with or which confer desirable properties, or that encode proteins with or which confer desirable properties. Following diversification by one or more of the methods herein, or otherwise available to one of skill, any nucleic acids that are produced can be selected for a desired activity or property, e.g. an HIV-1 virus encoded by the recombinant or chimeric HIV-1 nucleic acid exhibits enhanced replication in non-human mammalian cells. This can include identifying any activity that can be detected, for example, in an automated or automatable format, by any of the assays in the art, e.g., see Examples. A variety of related (or even unrelated) properties can be evaluated, in serial or in parallel, at the discretion of the practitioner.

Descriptions of a variety of diversity generating procedures for generating modified nucleic acid sequences, e.g., recombinant or chimeric HIV-1 nucleic acids, are found in the following publications and the references cited therein: Soong, N. et al. (2000) “Molecular breeding of viruses” Nat Genet 25(4):436-439; Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nuc. Acids Res. 14: 6361-6372; Wells et al. (1985) “Cassette mutagenesis: an efficient method for generation of multiple mutations at defined sites” Gene 34:315-323; and Grundström et al. (1985) “Oligonucleotide-directed mutagenesis by microscale ‘shot-gun’ gene synthesis” Nucl. Acids Res. 13: 3305-3316), double-strand break repair (Mandecki (1986) “Oligonucleotide-directed double-strand break repair in plasmids of Escherichia coli: a method for site-specific mutagenesis” Proc. Natl. Acad. Sci. USA, 83:7177-7181; and Arnold (1993) “Protein engineering for unusual environments” Current Opinion in Biotechnology 4:450-455). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications and applications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination;” WO 00/18906 by Patten et al., “Shuffling of Codon-Altered Genes;” WO 00/04190 by del Cardayre et al. “Evolution of Whole Cells and Organisms by Recursive Recombination;” WO 00/42561 by Crameri et al., “Oligonucleotide Mediated Nucleic Acid Recombination;” WO 00/42559 by Selifonov and Stemmer “Methods of Populating Data Structures for Use in Evolutionary Simulations;” WO 00/42560 by Selifonov et al., “Methods for Making Character Strings, Polynucleotides & Polypeptides Having Desired Characteristics;” WO 01/23401 by Welch et al., “Use of Codon-Varied Oligonucleotide Synthesis for Synthetic Shuffling;” and PCT/US01/06775 “Single-Stranded Nucleic Acid Template-Mediated Recombination and Nucleic Acid Fragment Isolation” by Affholter.

In brief, several different general classes of sequence modification methods, such as mutation, recombination, etc. are applicable to the present invention and set forth, e.g., in the references above. That is, the recombinant or chimeric HIV-1 nucleic acids of the invention can be modified to produce further recombinant or chimeric HIV-1 nucleic acids.

The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., WO 00/04190 by del Cardayre et al., also entitled “Evolution of Whole Cells and Organisms by Recursive Sequence Recombination.”

Synthetic recombination methods can also be used, in which oligonucleotides corresponding to targets of interest are synthesized and reassembled in PCR or ligation reactions that include oligonucleotides that correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., WO 00/42561 by Crameri et al., “Oligonucleotide Mediated Nucleic Acid Recombination;” WO 01/23401 by Welch et al., “Use of Codon-Varied Oligonucleotide Synthesis for Synthetic Shuffling;” WO 00/42560 by Selifonov et al., “Methods for Making Character Strings, Polynucleotides and Polypeptides Having Desired Characteristics;” and WO 00/42559 by Selifonov and Stemmer “Methods of Populating Data Structures for Use in Evolutionary Simulations.”

In silico methods of recombination can be effected in which genetic algorithms are used in a computer to recombine sequence strings that correspond to homologous (or even non-homologous) nucleic acids. The resulting recombined sequence strings are optionally converted into nucleic acids by synthesis of nucleic acids that correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in WO 00/42560 by Selifonov et al., “Methods for Making Character Strings, Polynucleotides and Polypeptides Having Desired Characteristics” and WO 00/42559 by Selifonov and Stemmer “Methods of Populating Data Structures for Use in Evolutionary Simulations.” Extensive details regarding in silico recombination methods are found in these applications. This methodology is generally applicable to the present invention in providing for recombination of the recombinant or chimeric HIV-1 nucleic acids in silico and/or the generation of corresponding nucleic acids or proteins.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “Single-Stranded Nucleic Acid Template-Mediated Recombination and Nucleic Acid Fragment Isolation” by Affholter, PCT/US01/06775.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences that hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other interculators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants that can optionally serve as a substrate for one or more in vitro or in vivo recombination methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods that result in the alteration of individual nucleotides or groups of contiguous or non-contiguous nucleotides can be favorably employed to introduce nucleotide diversity. The recombinant or chimeric HIV-1 nucleic acids of the invention can be used as substrates for these mutational methods, to produced further recombinant or chimeric HIV-1 nucleic acids. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process that involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids that lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, comprising genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence-similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “Methods for Generating and Screening Novel Metabolic Pathways,” and Thompson, et al. (1998) U.S. Pat. No. 5,824,485 Methods for Generating and Screening Novel Metabolic Pathways) and their use to identify protein activities of interest has been proposed (In addition to the references noted above, see, Short (1999) U.S. Pat. No. 5,958,672 “Protein Activity Screening of Clones Having DNA from Uncultivated Microorganisms”). Multispecies expression libraries include, in general, libraries comprising cDNA or genomic sequences from a plurality of species or strains, operably linked to appropriate regulatory sequences, in an expression cassette. The cDNA and/or genonic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to diversifying according to any of the methods described herein.

Libraries can be biased towards nucleic acids that encode proteins with desirable enzyme activities. For example, after identifying a clone from a library that exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “Production of Enzymes Having Desired Activities by Mutagenesis.” Desired activities can be identified by any method known in the art. For example, WO 99/10539 proposes that gene libraries can be screened by combining extracts from the gene library with components obtained from metabolically rich cells and identifying combinations that exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids that have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 51F to lambda repressor” J. Biol. Chem. 264:13355-60); and “Walk-Through Mutagenesis” (Crea, R; U.S. Pat. Nos. 5,830,650 and 5,798,208, and EP Patent 0527809 B1.

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

Nucleic Acids Used For Expression of Polypeptides

In accordance with the present invention, polynucleotide sequences which encode recombinant or chimeric HIV-1 polypeptides, fragments of HIV-1 polypeptides, related fusion polypeptides or proteins, or functional equivalents thereof, collectively referred to herein as “recombinant or chimeric HIV-1 polypeptides,” are used in recombinant DNA molecules that direct the expression of the recombinant or chimeric HIV-1 polypeptides in appropriate host cells.

Following transduction of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well known to those skilled in the art.

As noted, many references are available for the culture and production of many cells, including cells of bacterial, plant, animal (especially mammalian) and archebacterial origin. See, e.g., Sambrook, Ausubel, and Berger (all supra), as well as Freshney (1994) Culture of Animal Cells, a Manual of Basic Technique, 3^{rd} ed., Wiley-Liss, New York and the references cited therein; Doyle and Griffiths (1997) Mammalian Cell Culture: Essential Techniques, John Wiley and Sons, NY; Humason (1979) Animal Tissue Techniques, 4^{th} ed. W.H. Freeman and Company; and Ricciardelli et al. (1989) In vitro Cell Dev. Biol. 25:1016-1024. For plant cell culture and regeneration, Payne et al. (1992) Plant Cell and Tissue Culture in Liquid Systems, John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York) and Plant Molecular Biology (1993) R. R. D. Croy, Ed. Bios Scientific Publishers, Oxford, U.K. ISBN 0 12 198370 6. Cell culture media in general are set forth in Atlas and Parks (eds.) The Handbook of Microbiological Media (1993) CRC Press, Boca Raton, Fla. Additional information for cell culture is found in available commercial literature such as the Life Science Research Cell Culture Catalogue (1998) from Sigma-Aldrich, Inc (St Louis, Mo.) (“Sigma-LSRCCC”) and, e.g., the Plant Culture Catalogue and supplement (1997) also from Sigma-Aldrich, Inc. (St. Louis, Mo.) (“Sigma-PCCS”).

Polypeptides of the invention can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature protein. Finally, high performance liquid chromatography (HPLC) can be employed in the final purification steps. In addition to the references noted supra, a variety of purification methods are well known in the art, including, e.g., those set forth in Sandana (1997) Bioseparation of Proteins, Academic Press, Inc.; and Bollag et al. (1996) Protein Methods, 2nd Edition Wiley-Liss, NY; Walker (1996) The Protein Protocols Handbook, Humana Press, NJ, Harris and Angal (1990) Protein Purification Applications: A Practical Approach, IRL Press at Oxford, Oxford, England; Harris and Angal, Protein Purification Methods: A Practical Approach, IRL Press at Oxford, Oxford, England; Scopes (1993) Protein Purification: Principles and Practice, 3 ed. Springer Verlag, NY; Janson and Ryden (1998) Protein Purification: Principles, High Resolution Methods and Applications, 2d ed. Wiley-VCH, NY; and Walker (1998) Protein Protocols on CD-ROM Humana Press, NJ.

Cell-free transcription/translation systems can also be employed to produce polypeptides using DNAs or RNAs of the present invention. Several such systems are commercially available. A general guide to in vitro transcription and translation protocols is found in Tymms (1995) In vitro Transcription and Translation Protocols: Methods in Molecular Biology, Volume 37, Garland Publishing, NY.

Antisense Technology/RNA Suppression

The nucleic acids encoding any of the chimeric or recombinant polypeptides of the invention, including, e.g., SEQ ID NOS:8-39 or a polypeptide sequence having at least about 80%, 85%, 90%, 95%, 96%, 97%, 98%, or 99% more amino acid sequence identity to any one of SEQ ID NOS:8-39, are also useful for sense and anti-sense suppression of expression or, e.g. to down-regulate expression of a nucleic acid of the invention, once expression of the nucleic acid is no-longer desired in the cell, and/or for interfering RNA silencing technology in which an RNA sequence (e.g., a chimeric or recombinant RNA sequence of the invention) is used to suppress or prevent replication in any cell type. Similarly, the nucleic acids of the invention, or subsequences or anti-sense sequences thereof, can also be used to block expression of naturally occurring homologous nucleic acids. A variety of sense and anti-sense technologies are known in the art, e.g., as set forth in Lichtenstein and Nellen (1997) Antisense Technology: A Practical Approach, IRL Press at Oxford University, Oxford, England, and in Agrawal (1996) Antisense Therapeutics, Humana Press, NJ, and the references cited therein.

Probes

Also contemplated are uses of polynucleotides, also referred to herein as oligonucleotides, typically having at least 12 bases, preferably at least 15, more preferably at least 20, 30, or 50 bases, which hybridize under highly stringent conditions to a recombinant or chimeric HIV-1 polynucleotide sequence described above. The polynucleotides can be used as probes, primers, sense and antisense agents, and the like, according to methods as noted supra.

Uses of Recombinant or Chimeric Polypeptides of the Invention

Adjuvants

In one aspect, the recombinant or chimeric HIV-1 polypeptides of the present invention or fragments thereof are useful as adjuvants to stimulate or augment an immune response related to an antigen delivered by a retroviral vector (e.g., DNA vaccine) incorporating the recombinant or chimeric HIV-1 polypeptide to a target tissue, cell, or organ. In another aspect, the invention provides methods for administering one or more of the invention described above to a subject, including an organism or mammal, including, e.g., a human, primate, mouse, pig, cow, goat, rabbit, rat, guinea pig, hamster, horse, sheep; or a non-mammalian vertebrate such as a bird (e.g., a chicken or duck) or a fish, or invertebrate.

Antibodies

In another aspect of the invention, a recombinant or chimeric HIV-1 polypeptide, or subsequence thereof, of the invention is used to produce antibodies which have, e.g., diagnostic and therapeutic and/or prophylactic uses, e.g., related to the activity, distribution, and expression of retrovirus sequences.

Antibodies to recombinant or chimeric HIV-1 polypeptides of the invention can be generated by methods well known in the art. Such antibodies can include, but are not limited to, polyclonal, monoclonal, chimeric, humanized, single chain, Fab fragments and fragments produced by an Fab expression library. Antibodies, i.e., those that block receptor binding, are especially preferred for therapeutic and/or prophylactic use.

Recombinant or chimeric HIV-1 polypeptides for antibody induction do not require biological activity; however, the polypeptide or oligopeptide must be antigenic. Peptides used to induce specific antibodies can have an amino acid sequence comprising at least 10 amino acids, preferably at least 15 or 20 amino acids. S hort stretches of a recombinant or chimeric HIV-1 polypeptide can be fused with another protein, such as keyhole limpet hemocyanin, and antibody produced against the chimeric molecule.

Methods of producing polyclonal and monoclonal antibodies are known to those of ordinary skill in the art, and many antibodies are available. See, e.g., Coligan (1991) Current Protocols in Immunology Wiley/Greene, NY; and Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, N.Y.; and Kohler and Milstein (1975) Nature 256:495-497. Other suitable techniques for antibody preparation include selection of libraries of recombinant antibodies in phage or similar vectors. See Huse et al. (1989) Science 246:1275-1281; and Ward et al. (1989) Nature 341:544-546. Specific monoclonal and polyclonal antibodies and antisera (or antiserum) will usually bind with a K_{D} of at least about 0.1 μM, preferably at least about 0.01 μM or better, and most typically and preferably, 0.001 μM or better.

Detailed methods for preparation of chimeric (humanized) antibodies can be found in U.S. Pat. No. 5,482,856. Additional details on humanization and other antibody production and engineering techniques can be found in Borrebaeck (ed) (1995) Antibody Engineering, 2^{nd} Edition Freeman and Company, NY (Borrebaeck); McCafferty et al. (1996) Antibody Engineering, A Practical Approach IRL at Oxford Press, Oxford, England (McCafferty), and Paul (1995) Antibody Engineering Protocols Humana Press, Towata, N.J. (Paul).

In one useful embodiment, this invention provides for fully humanized antibodies against the recombinant or chimeric HIV-1 polypeptides of the invention. Humanized antibodies are especially desirable in applications where the antibodies are used as therapeutics or prophylactics in vivo in human patients (or ex vivo in cells obtained from human patients). Human antibodies consist of characteristically human immunoglobulin sequences. The human antibodies of this invention can be produced in using a wide variety of methods (see, e.g., Larrick et al., U.S. Pat. No. 5,001,065, and Borrebaeck McCafferty and Paul, supra, for a review). In one embodiment, the human antibodies of the present invention are produced initially in trioma cells. Genes encoding the antibodies are then cloned and expressed in other cells, such as non-human mammalian cells. The general approach for producing human antibodies by trioma technology is described by Ostberg et al. (1983), Hybridoma 2:361-367, Ostberg, U.S. Pat. No. 4,634,664, and Engelman et al., U.S. Pat. No. 4,634,666. The antibodyproducing cell lines obtained by this method are called triomas because they are descended from three cells; two human and one mouse. Triomas have been found to produce antibody more stably than ordinary hybridomas made from human cells.

Therapeutic and Prophylactic Compositions

Other aspects of the invention relate to compositions comprising a recombinant or chimeric HIV-1 nucleic acid and/or virus and/or polypeptide of the invention and an excipient. In one embodiment, the excipient is a pharmaceutically acceptable excipient. These compositions are useful for, e.g., the development and/or study of vaccines, e.g., attenuated vaccines and subunit vaccines, in humans and other mammals.

The polynucleotides and/or polypeptides of the invention can be employed for therapeutic and/or prophylactic uses and treatment methods in combination with a carrier or excipient, including a suitable pharmaceutical excipient. Such compositions comprise, respectively, a therapeutically or prophylactically effective amount of at least one polynucleotide and/or polypeptide of the invention, and a carrier or excipient. The invention also provides pharmaceutical compositions that comprise therapeutically or prophylactically effective amount of at least one polynucleotide and/or polypeptide of the invention, and a pharmaceutically acceptable carrier or pharmaceutically acceptable excipient. The carrier or excipient includes, but is not limited to, saline, buffered saline, dextrose, water, glycerol, ethanol, and combinations thereof. The formulation should suit the mode of administration. Methods of administering nucleic acids, polypeptides and proteins are well known in the art, and further discussed below.

Therapeutic and/or prophylactic compositions comprising one or more retroviruses comprising an HIV-1 polypeptide(s) of the invention, or retrovirus HIV-1 polypeptide(s) of the invention are tested in appropriate in vitro, ex vivo, and in vivo animal models of disease, to confirm efficacy, tissue metabolism, and to estimate dosages, according to methods well known in the art. In particular, dosages can be determined by activity comparison of the retroviruses comprising the HIV-1 polypeptides of the invention, e.g., to existing retroviral vectors, i.e., in a relevant assay. Typically, retroviruses having the HIV-1 polypeptides of the invention and further comprising a therapeutic and/or prophylactic gene construct are administered, e.g., for gene therapy.

A “therapeutic treatment” is a treatment administered to a subject who displays symptoms or signs of a pathology, disease, or disorder, in which treatment is administered to the subject for the purpose of diminishing or eliminating those signs or symptoms of the pathology, disease, or disorder. A “therapeutic activity” is an activity of an agent, such as a nucleic acid, vector, gene, polypeptide, protein, substance, or composition thereof, that eliminates or diminishes signs or symptoms of a pathology, disease or disorder, when administered to a subject suffering from such signs or symptoms. This effect is termed a “therapeutic effect.” A “therapeutically useful” agent or compound (e.g., nucleic acid or polypeptide) indicates that an agent or compound is useful in diminishing, treating, or eliminating such signs or symptoms of a pathology, disease or disorder.

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as a nucleic acid, vector, gene, polypeptide, protein, substance, or composition thereof that, when administered to a subject who does not display signs or symptoms of a pathology, disease or disorder, or who displays only early signs or symptoms of a pathology, disease, or disorder, diminishes, prevents, or decreases the risk of the subject developing the pathology, disease, or disorder. This effect is termed a “prophylactic effect.” A “prophylactically useful” agent or compound (e.g., nucleic acid or polypeptide) refers to an agent or compound that is useful in diminishing, preventing, treating, or decreasing development of a pathology, disease or disorder.

Genetic Vectors

Gene therapy and genetic vaccine vectors are useful for treating and/or preventing various diseases and other conditions. The following discussion focuses on the on the use of vectors because gene therapy and genetic vaccine method typically employ vectors, but persons of skill in the art appreciate that the nucleic acids of the invention can, depending on the particular application, be employed in the absence of vector sequences. Accordingly, references in the following discussion to vectors should be understood as also relating to nucleic acids of the invention that lack vector sequences.

Vectors can be delivered to a subject to induce an immune response or other therapeutic or prophylactic response. Suitable subjects include, but are not limited to, a mammal, including, e.g., a human, primate, mouse, monkey, orangutan, baboon, mouse, pig, cow, cat, goat, rabbit, rat, guinea pig, hamster, horse, sheep; or a non-mammalian vertebrate such as a bird (e.g., a chicken or duck) or a fish, or invertebrate.

Vectors can be delivered in vivo by administration to an individual patient, typically by local (direct) administration or by systemic administration (e.g., intravenous, intraperitoneal, intramuscular, subdermal, intracranial, anal, vaginal, oral, buccal route or they can be inhaled) or they can be administered by topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

In local (direct) administration formats, the nucleic acid or vector is typically administered or transferred directly to the cells to be treated or to the tissue site of interest (e.g., tumor cells, tumor tissue sample, organ cells, blood cells, cells of the skin, lung, heart, muscle, brain, mucosae, liver, intestine, spleen, stomach, lymphatic system, cervix, vagina, prostate, mouth, tongue, etc.) by any of a variety of formats, including topical administration, injection (e.g., by using a needle or syringe), or vaccine or gene gun delivery, pushing into a tissue, organ, or skin-site. For standard gene gun administration, the vector or nucleic acid of interest is precipitated onto the surface of microscopic metal beads. The microprojectiles are accelerated with a shock wave or expanding helium gas, and penetrate tissues to a depth of several cell layers. For example, the Accel™ Gene Delivery Device manufactured by Agacetus, Inc. Middleton Wis. is suitable for use in this embodiment. The nucleic acid or vector can be delivered, for example, intramuscularly, intradermally, subdermally, subcutaneously, orally, intraperitoneally, intrathecally, intravenously, or placed within a cavity of the body (including, e.g., during surgery), or by inhalation or vaginal or rectal administration.

In in vivo indirect contact/administration formats, the nucleic acid or vector is typically administered or transferred indirectly to the cells to be treated or to the tissue site of interest, including those described above (such as, e.g., skin cells, organ systems, lymphatic system, or blood cell system, etc.), by contacting or administering the nucleic acid or vector of the invention directly to one or more cells or population of cells from which treatment can be facilitated. For example, tumor cells within the body of the subject can be treated by contacting cells of the blood or lymphatic system, skin, or an organ with a sufficient amount of the polypeptide such that delivery of the nucleic acid or vector to the site of interest (e.g., tissue, organ, or cells of interest or blood or lymphatic system within the body) occurs and effective prophylactic or therapeutic treatment results. Such contact, administration, or transfer is typically made by using one or more of the routes or modes of administration described above.

A large number of delivery methods are well known to those of skill in the art. Such methods include, for example liposome-based gene delivery (Debs and Zhu (1993) WO 93/24640; Mannino and Gould-Fogerite (1988) BioTechniques 6(7):682-691; Rose U.S. Pat. No. 5,279,833; Brigham (1991) WO 91/06309; and Felgner et al. (1987) Proc. Natl Acad. Sci. USA 84:7413-7414), as well as use of viral vectors (e.g., adenoviral (see, e.g., Berns et al. (1995) Ann. NY Acad. Sci. 772:95-104; Ali et al. (1994) Gene Ther. 1:367-384; and Haddada et al. (1995) Curr. Top. Microbiol. Immunol. 199 (Pt 3):297-306 for review), papillomaviral, retroviral (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al., Gene Therapy (1994) supra.), and adeno-associated viral vectors (see, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 and Samulski (supra) for an overview of AAV vectors; see also, Lebkowski, U.S. Pat. No. 5,173,414; Tratschin et al. (1985) Mol. Cell. Biol. 5(11):3251-3260; Tratschin, et al. (1984) Mol. Cell. Biol., 4:2072-2081; Hermonat and Muzyczka (1984) Proc. Natl Acad. Sci. USA, 81:6466-6470; McLaughlin et al. (1988) and Samulski et al. (1989) J. Virol., 63:03822-3828), and the like.

“Naked” DNA and/or RNA that comprises a genetic vaccine can also be introduced directly into a tissue, such as muscle, by injection using a needle or other similar device. See, e.g., U.S. Pat. No. 5,580,859. Other methods such as “biolistic” or particle-mediated transformation (see, e.g., Sanford et al., U.S. Pat. No. 4,945,050; U.S. Pat. No. 5,036,006) are also suitable for introduction of genetic vaccines into cells of a mammal according to the invention. These methods are useful not only for in vivo introduction of DNA into a subject, such as a mammal, but also for ex vivo modification of cells for reintroduction into a mammal. DNA is conveniently introduced directly into the cells of a mammal or other subject using, e.g., injection, such as via a needle, or a “gene gun.” As for other methods of delivering genetic vaccines, if necessary, vaccine administration is repeated in order to maintain the desired level of immunomodulation, such as the level or response of T cell activation or T cell proliferation, or antibody titer level. Alternatively, nucleotides can be impressed into the skin of the subject.

Gene therapy and genetic vaccine vectors (e.g., DNA, plasmids, expression vectors, adenoviruses, liposomes, papillomaviruses, retroviruses, etc.) comprising at least one nucleic acid sequence of the invention can be administered directly to the subject (usually a mammal) for transduction of cells in vivo. The vectors can be formulated as pharmaceutical compositions for administration in any suitable manner, including parenteral (e.g., subcutaneous, intramuscular, intradermal, or intravenous), topical, oral, rectal, vaginal, intrathecal, buccal (e.g., sublingual), or local administration, such as by aerosol or transdermally, for immunotherapeutic or other prophylactic and/or therapeutic treatment. Pretreatment of skin, for example, by use of hair-removing agents, can be useful in transdermal delivery. Suitable methods of administering such packaged nucleic acids are available and well known to those of skill in the art, and, although more than one route can be used to administer a particular composition, a particular route can often provide a more immediate and more effective reaction than another route.

Ex Vivo Applications

Ex vivo methods for introducing a therapeutic or prophylactic gene into a cell or organism frequently involve transducing the cell ex vivo with a therapeutic or prophylactic nucleic acid or gene construct of this invention, respectively, and introducing the cell into the organism. Target cells include CD4+ cells such as CD4+ T cells or macrophage isolated or cultured from a patient, stem cells, or the like. See, e.g., Freshney et al., supra, and the references cited therein for a discussion of how to isolate and culture cells from patients. Alternatively, the cells can be those stored in a cell bank (e.g., a blood bank). In one class of embodiments, the packageable nucleic acid encodes an anti-viral therapeutic agent (e.g., suicide gene, trans-dominant gene, anti-viral ribozyme, anti-sense gene, or decoy gene) which inhibits the growth or replication of a cell infected with a virus (e.g., HIV), or a virus, under the control of an activated or constitutive promoter. The cell transformation vector inhibits viral replication in any of those cells already infected with the subject virus, in addition to conferring a protective effect to cells that are not infected with the virus. Thus, the present invention provides a method of protecting cells in vitro, ex vivo or in vivo, even when the cells are already infected with the virus against which protection is sought. Alternatively, the packageable nucleic acid encodes a therapeutic or prophylactic gene construct directed against a non-viral infectious agent. In other embodiments the packageable gene construct is selected to provide anti-oncogenic, or other anticancer, e.g., anti-metastatic, effects. In yet other embodiments, the therapeutic gene construct provides remediation or prophylaxis for a congenital or inborn error, e.g., of metabolism.

In some embodiments, stem cells (which are typically not CD4+) are used in ex vivo procedures for cell transformation and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-g and TNF-a are known (see Inaba et al. (1992) J. Exp. Med. 176, 1693-1702, and Szabolcs et al. (1995), J. Immunol. 154:5851-5861). Methods of pseudotyping retrovirus vectors so that they can transform stem cells are described above. An affinity column isolation procedure can be used to isolate cells that bind to CD34, or to antibodies bound to CD34. See Ho et al. (1995) Stem Cells 13 (suppl. 3):100-105. See also Brenner (1993) Journal of Hematotherapy 2:7-17. In another embodiment, hematopoietic stem cells are isolated from fetal cord blood. Yu et al. (1995) Proc. Nat'l Acad. Sci. USA 92:699-703 describe a preferred method of transducing CD34+ cells from human fetal cord blood using retroviral vectors. Rather than using stem cells, T cells can also be transduced in ex vivo procedures. Several techniques are known for isolating T cells. In one method, Ficoll-Hypaque density gradient centrifugation is used to separate PBMC from red blood cells and neutrophils according to established procedures. Cells are washed with modified AIM-V (which comprises AIM-V (GIBCO) with 2 mM glutamine, 10 mg/ml gentamicin sulfate, 50 mg/ml streptomycin) supplemented with 1% fetal bovine serum (FBS). Enrichment for T cells is performed by negative or positive selection with appropriate monoclonal antibodies coupled to columns or magnetic beads according to standard techniques. An aliquot of cells is analyzed for desired cell surface phenotype (e.g., CD4, CD8, CD3, CD14, etc.).

In general, the expression of surface markers facilitates identification and purification of T cells. Methods of identification and isolation of T cells include FACS, column chromatography, panning with magnetic beads, western blots, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunoabsorbent assays (ELISAs), immunofluorescent assays, and the like. For a review of immunological and immunoassay procedures in general, see Stites and Terr (eds.) 1991 Basic and Clinical Immunology (7th ed.) and Paul, supra. For a discussion of how to make antibodies to selected antigens see, e.g., Coligan (1991) Current Protocols in Immunology Wiley/Greene, NY; and Harlow and Lane (1989) Antibodies: A Laboratory Manual Cold Spring Harbor Press, NY; Stites et al. (eds.) Basic and Clinical Immunology (4th ed.).

In addition to the ex vivo uses described above, the packaging cell lines of the invention and the packageable nucleic acids of the invention are useful generally in cloning methods. Packageable nucleic acids are packaged in an retrovirus particle and used to transform an infectible cell (e.g., 293T cells) in vitro, or ex vivo or in vivo. This provides one of ordinary skill in the art with a technique and vectors for transforming cells with a nucleic acid of choice, e.g., in drug discovery assays, or as a tool in the study of gene regulation, or as a general cloning vector.

In Vivo or Ex Vivo Transformation

Non-primate retroviral particles containing therapeutic or prophylactic nucleic acids can be administered directly to a cell or an organism for transduction of cells in vivo or to cells of the organism ex vivo. In one aspect, the invention provides methods comprising administering one or more nucleotides of the invention described above to a mammal, including, e.g., a human, primate, mouse, pig, cow, goat, rabbit, rat, guinea pig, hamster, horse, sheep; or a non-mammalian vertebrate such as a bird (e.g., a chicken or duck) or a fish, or invertebrate.

Administration is by any of the routes normally used for introducing a molecule into ultimate contact with blood or tissue cells. The polypeptides and polynucleotides of the invention, and vectors, cells, and compositions comprising such molecules, are administered in any suitable manner, including, in some aspects, with a pharmaceutically acceptable carrier. Suitable methods of administering such molecules, in the context of the present invention, to a subject are available, and, although more than one route can be used to administer a particular composition, a particular route can often provide a more immediate and more effective reaction than another route. Preferred routes are readily ascertained by those of skill in the art.

Packageable nucleic acids, e.g., recombinant or chimeric HIV-1 nucleic acids of the invention are used to treat and prevent a variety of diseases, including virally mediated diseases, cancer, and other genetic disorders, in animals and human patients. The packaged nucleic acids are administered in any suitable manner, preferably with pharmaceutically acceptable carriers. Suitable methods of administering such packaged nucleic acids in the context of the present invention to a subject, e.g., patient, are available, and, although more than one route can be used to administer a particular composition, a particular route can often provide a more immediate and more effective reaction than another route.

Compositions comprising cells expressing at least one full length form of a polypeptide of the invention or a fragment thereof are also a feature of the invention. Such cells are readily prepared as described herein by transfection with DNA plasmid vector encoding at least one of the polypeptide of the invention. Compositions of such cells can comprise a pharmaceutically composition comprising a pharmaceutically acceptable carrier or excipient.

Pharmaceutical compositions of the invention can, but need not, include a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention. A variety of aqueous carriers can be used, e.g., buffered saline, such as PBS, and the like. These solutions are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of gene therapy or genetic vaccine vector in these formulations can vary widely, and will be selected primarily based on fluid volumes, viscosities, body weight and the like in accordance with the particular mode of administration selected and the patient's needs.

Compositions comprising polypeptides and polynucleotides, and vectors, cells, and other formulations comprising these and other components of the invention, can be administered by a number of routes including, but not limited to oral, intranasal, intravenous, intraperitoneal, intramuscular, transdermal, subcutaneous, intradermal, topical, sublingual, vaginal, or rectal means. Polypeptide and nucleic acid compositions can also be administered via liposomes. Such administration routes and appropriate formulations are generally known to those of skill in the art.

The polypeptide or polynucleotide of the invention or fragment thereof, or vector comprising a nucleic acid of the invention, alone or in combination with other suitable components, can also be made into aerosol formulations (e.g., they can be “nebulized”) to be administered via inhalation. Aerosol formulations can be placed into pressurized acceptable propellants, such as dichlorodifluoromethane, propane, nitrogen, and the like.

Formulations suitable for oral administration can comprise (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, tragacanth, microcrystalline cellulose, acacia, gelatin, colloidal silicon dioxide, croscarmellose sodium, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art. It is recognized that the gene therapy vectors and genetic vaccines, when administered orally, must be protected from digestion. This is typically accomplished either by complexing the vector with a composition to render it resistant to acidic and enzymatic hydrolysis or by packaging the vector in an appropriately resistant carrier such as a liposome. Means of protecting vectors from digestion are well known in the art. The pharmaceutical compositions can be encapsulated, e.g., in liposomes, or in a formulation that provides for slow release of the active ingredient. Suitable formulations for rectal administration include, for example, suppositories, which comprise the packaged nucleic acid with a suppository base. Suitable suppository bases include natural or synthetic triglycerides or paraffin hydrocarbons. In addition, it is also possible to use gelatin rectal capsules that comprise a combination of the packaged nucleic acid with a base, including, for example, liquid triglycerides, polyethylene glycols, and paraffin hydrocarbons.

Pharmaceutically acceptable carriers are determined in part by the particular composition being administered, as well as by the particular method used to administer the composition. Accordingly, there is a wide variety of suitable formulations of pharmaceutical compositions of the present invention.

Retrovirus particles incorporating therapeutic or prophylactic gene constructs can be administered by a number of routes including, but not limited to oral, intravenous, intraperitoneal, intramuscular, transdermal, subcutaneous, topical, sublingual, or rectal means. Such administration routes and appropriate formulations are generally known to those of ordinary skill in the art.

The packaged nucleic acids, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations of packaged nucleic acid can be presented in unit-dose or multidose sealed containers, such as ampules and vials.

Injection solutions and suspensions can be prepared from sterile powders, granules, and tablets of the kind previously described. Cells transduced by the packaged nucleic acid can also be administered intravenously or parenterally.

Cells transduced with the nucleic acids of the invention as described herein in the context of ex vivo or in vivo therapy can also be administered intravenously or parenterally. It will be appreciated that the delivery of cells to patients is routine, e.g., delivery of cells to the blood via intravenous, intramuscular, or intraperitoneal administration or other common route. Cells transduced as described above in the context of ex vivo therapy can also be administered intravenously or parenterally as described above. It will be appreciated that the delivery of cells to subject, e.g., humans (patients), mammals, or other animals, is routine, e.g., delivery of cells to the blood via intravenous or intraperitoneal administration.

The dose administered to a patient, in the context of the present invention is sufficient to effect a beneficial therapeutic or prophylactic response in the patient over time. The dose will be determined by the efficacy of the particular therapeutic or prophylactic gene construct, or formulation, and the titer or infectivity of the retroviruses employed and the condition of the patient, as well as the body weight or surface area of the patient to be treated. The size of the dose also will be determined by the existence, nature, and extent of any adverse side-effects that accompany the administration of a particular vector, formulation, transduced cell type or the like in a particular patient. Dosages to be used for therapeutic or prophylactic treatment of a particular disease or disorder can be determined by one of skill by comparison to those dosages used for existing therapeutic or prophylactic treatment protocols for the same disease or disorder.

In determining the effective amount of the vector, cell type, or formulation to be administered in the treatment or prophylaxis of inborn errors of metabolism, cancers, or infections, the physician evaluates circulating plasma levels, vector/cell/formulation/toxicities, progression of the disease, and the production of antibodies directed against the vector or other aspect of the therapeutic or prophylactic composition.

The dose administered, e.g., to a 70 kilogram patient, will be in the range equivalent to dosages of currently-used retroviral gene therapies, and doses are calculated to yield an equivalent amount of therapeutic or prophylactic nucleic acid or expressed protein. In addition to remediating hereditary disorders, the vectors of this invention can be used to supplement treatment of cancers and virally-mediated conditions by any known conventional therapy, including cytotoxic agents, nucleotide analogues (e.g., when used for treatment of HIV infection), biologic response modifiers, and the like.

In one aspect, for example, in determining the effective amount of the vector to be administered in the treatment or prophylaxis of an infection or other condition, wherein the vector comprises any nucleic acid sequence of the invention described herein or encodes any polypeptide of the invention described herein, the physician evaluates vector toxicities, progression of the disease, and the production of anti-vector antibodies, if any. In one aspect, the dose equivalent of a naked nucleic acid from a vector for a typical 70 kilogram patient can range from about 10 ng to about 1 g, about 100 ng to about 100 mg, about 1 μg to about 10 mg, about 10 μg to about 1 mg, or from about 30-400 μg. Doses of vectors used to deliver the nucleic acid are calculated to yield an equivalent amount of therapeutic nucleic acid. Administration can be accomplished via single or divided doses.

In therapeutic applications, compositions are administered to a patient suffering from a disease in an amount sufficient to cure or at least partially arrest or ameliorate the disease or at least one of its complications. An amount adequate to accomplish this is defined as a “therapeutically effective dose.” Amounts effective for this use will depend upon the severity of the disease and the general state of the patient's health. Single or multiple administrations of the compositions can be administered depending on the dosage and frequency as required and tolerated by the patient. In any event, the composition should provide a sufficient quantity of protein to effectively treat the patient.

In prophylactic applications, compositions are administered to a human or other mammal to induce an immune or other prophylactic response that can help protect against the establishment of an infectious disease, cancer, autoimmune disorder, or other condition.

For administration, the retroviral vectors and transduced cells of the present invention can be administered at a rate determined by the LD-50 of the therapeutic or prophylactic gene construct, vector, or transduced cell type, and the side-effects of the therapeutic or prophylactic compositions, vector or cell type at various concentrations, as applied to the mass and overall health of the patient. Administration can be accomplished via single or divided doses.

The toxicity and therapeutic efficacy of the vectors that include molecules of the invention are determined using standard pharmaceutical procedures in cell cultures or experimental animals. One can determine the LD₅₀ (the dose lethal to 50% of the population) and the ED₅₀ (the dose therapeutically effective in 50% of the population) using procedures presented herein and those otherwise known to those of skill in the art. Nucleic acids, polypeptides, proteins, fusion proteins, transduced cells and other formulations of the present invention can be administered at a rate determined, e.g., by the LD₅₀ of the formulation, and the side-effects thereof at various concentrations, as applied to the mass and overall health of the patient. Again, administration can be accomplished via single or divided doses.

A typical pharmaceutical composition for intravenous administration would be about 0.1 to 10 mg per patient per day. Dosages from 0.1 up to about −100 mg per patient per day can be used, particularly when the drug is administered to a secluded site and not into the blood stream, such as into a body cavity or into a lumen of an organ. Substantially higher dosages are possible in topical administration. For recombinant promoters of the invention that express the linked transgene at high levels, it can be possible to achieve the desired effect using lower doses, e.g., on the order of about 1 μg or 10 μg per patient per day. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

For introduction of recombinant retrovirus infected cells into a patient, blood samples are obtained prior to infusion, and saved for analysis. Between 1×10⁶ and 1×10¹² transduced cells are infused intravenously over 60-200 minutes. Vital signs and oxygen saturation by pulse oximetry are closely monitored. Blood samples are obtained 5 minutes and 1 hour following infusion and saved for subsequent analysis. Leukopheresis, transduction and reinfusion are optionally repeated every 2 to 3 months for a total of 4 to 6 treatments in a one year period. After the first treatment, infusions can be performed on a outpatient basis at the discretion of the clinician. If the reinfusion is given as an outpatient, the participant is monitored for at least 4, and preferably 8 hours following the therapy. Transduced cells are prepared for reinfusion according to established methods. See Abrahamsen et al. (1991) J. Clin. Apheresis 6:48-53; Carter et al. (1988) J. Clin. Arpheresis 4:113-117; Aebersold et al. (1988), J. Immunol. Methods 112:1-7; Muul et al. (1987) J. Immunol. Methods 101:171-181 and Carter et al. (1987) Transfusion 27:362-365. After a period of about 2-4 weeks in culture, the cells should number between 1×10⁶ and 1×10¹². In this regard, the growth characteristics of cells vary from patient to patient and from cell type to cell type. About 72 hours prior to reinfusion of the transduced cells, an aliquot is taken for analysis of phenotype, and percentage of cells expressing the therapeutic or prophylactic agent.

If a patient undergoing infusion of a vector or transduced cell or protein formulation develops fevers, chills, or muscle aches, he/she receives the appropriate dose of aspirin, ibuprofen, acetaminophen or other pain/fever controlling drug. Patients who experience reactions to the infusion such as fever, muscle aches, and chills are premedicated 30 minutes prior to the future infusions with either aspirin, acetaminophen, or, e.g., diphenhydramine. Meperidine is used for more severe chills and muscle aches that do not quickly respond to antipyretics and antihistamines. Cell infusion is slowed or discontinued depending upon the severity of the reaction.

The polypeptides and nucleic acids of the invention, and cells, vectors, transgenic animals, and compositions that comprise these molecules of the invention can be packaged in packs, dispenser devices, and kits for administration to a subject, such as a mammal. For example, packs or dispenser devices that contain one or more unit dosage forms are provided. Typically, instructions for administration of the compounds will be provided with the packaging, along with a suitable indication on the label that the compound is suitable for treatment of an indicated condition. For example, the label may state that the active compound within the packaging is useful for treating a particular infectious disease, autoimmune disorder, tumor, or for preventing or treating other diseases or conditions that are mediated by, or potentially susceptible to, a subject's or mammalian immune response.

Recombinant, Modified or Chimeric Viruses

The invention also relates to modified, recombinant, or chimeric viruses, including, e.g., HIV-1 virus variants. Accordingly, the invention provides a modified, recombinant, or chimeric virus comprising a recombinant or chimeric nucleic acid of the invention. In one embodiment, the invention provides a modifed or chimeric HIV-1 virus produced by expression or translation of a recombinant or chimeric HIV-1 nucleic acid of the invention in a population of primate cells. In a preferred embodiment, the invention provides a modified, recombinant, or chimeric virus produced by expression or translation of an RNA nucleic acid in a population of primate cells, the RNA nucleic acid comprising an RNA polynucleotide sequence comprising from at least about nucleic acid residue 530 to at least about nucleic acid residue 9859 of the polynucleotide sequence of a recombinant or chimeric nucleic acid of the invention, wherein each thymine is replaced by a uracil, or a complementary sequence of said RNA polynucleotide sequence. In another embodiment, the invention provides a modified, recombinant, or chimeric virus comprising an RNA polynucleotide sequence, said RNA polynucleotide sequence comprising from at least about nucleic acid residue 530 to at least about nucleic acid residue 9859 of the polynucleotide sequence of a recombinant or chimeric nucleic acid of the invention, wherein each thymine is replaced by a uracil, or a complementary sequence of said RNA polynucleotide sequence. In one variation of this embodiment, the invention provides a modified, recombinant, or chimeric virus comprising an RNA polynucleotide sequence, said RNA polynucleotide sequence comprising from at least about nucleic acid residue 530 to at least about nucleic acid residue 9859 of the polynucleotide sequence of the isolated, chimeric or recombinant nucleic acid of SEQ ID NO:1 to SEQ ID NO:7, each thymine is replaced by a uracil, or a complementary sequence of said RNA polynucleotide sequence.

In one aspect, modified or chimeric virus was produced using standard transfection of mammalian cells (e.g., 293 cells or the like) with proviral DNA encoding one or more recombinant, modified or chimeric polypeptides of the invention. Standard transfection techniques known to those of ordinary skill in the art were employed (see, Current Protocols in Molecular Biology, F. M. Ausubel et al., eds., Current Protocols, ajoint venture between Greene Publishing Associates, Inc. and John Wiley & Sons, Inc. (1994, supplemented through 1999) (hereinafter “Ausubel”)). Alternatively, modified or chimeric virus was produced from primate or other non-human mammalian cell lines infected with proviral DNA encoding one or more recombinant, modified or chimeric polypeptides of the invention.

In one embodiment, the modified or chimeric HIV-1 viruses described herein exhibit replication in macaque monkey cells in vivo. In a preferred embodiment, the macaque monkey cells comprise pig-tailed macaque monkey cells. In another embodiment, the modified or chimeric HIV-1 virus exhibits enhanced replication in macaque monkey cells compared to replication of an HIV-1 virus in macaque monkey cells. In preferred embodiments, the enhanced replication comprises growth to a higher titer and/or replication for a longer period of time and/or growth at a faster rate.

In another embodiment, the modified or chimeric viruses described herein, e.g., HIV-1 virus variants, exhibit replication in vivo in a macaque monkey. In preferred embodiments, the macaque monkey includes a pig-tailed macaque monkey. In further embodiments, the modified or chimeric HIV-1 viruses exhibit enhanced replication in vivo in the pig-tailed macaque monkey compared to replication of an HIV-1 virus in vivo in said macaque monkey.

The invention also provides cells comprising the modified or chimeric viruses described herein. In various embodiments, the cells include primate cells, human cells, or macaque monkey cells.

The invention also provides cell-culture derived recombinant or chimeric virus progeny of at least one recombinant or chimeric virus of the invention. To produce such virus progeny, a population of permissive primate or other mammalian cells was contacted with at least one recombinant or chimeric nucleic acid of the invention described above (e.g., that encodes at least one chimeric or recombinant polypeptide of the invention) so as to infect the population of cells. The cells were amplified and produced recombinant or chimeric virus progeny, which progeny viruses were then isolated from the cells or cell culture.

In one embodiment, the cell-culture derived progeny exhibit replication in a macaque monkey and the macaque monkey comprising the progeny exhibits at least one symptom of HIV infection.

Another aspect of the invention is an evolved HIV-1 virus. In one embodiment, the invention provides an evolved HIV-1 virus produced by passaging a viral isolate at least one time through macaque monkey cells, tissue, or blood, wherein the viral isolate comprises the recombinant or chimeric HIV-1 virus of the invention. In a preferred embodiment, a macaque monkey comprising the evolved HIV-1 virus develops at least one symptom of HIV infection. In another embodiment, the evolved HIV-1 virus exhibits enhanced replication in macaque monkey cells, tissue, or blood compared to replication in macaque monkey cells, tissue, or blood of the modified, recombinant, or chimeric HIV-1 virus prior to a first passage.

The invention also provides a method for producing an evolved HIV-1 virus that replicates and causes at least one symptom of HIV infection in a macaque monkey. The method entails passaging a viral isolate at least one time through macaque monkey cells, tissue, or blood, wherein the viral isolate comprises a recombinant or chimeric HIV-1 virus of the invention, wherein prior to a first passage the recombinant or chimeric HIV-1 virus of the invention includes a recombinant or chimeric HIV-1 nucleic acid of the invention.

In some aspects, recombinant or chimeric HIV-1 viruses of the invention are useful in therapeutic or prophylactic compositions, as described in detail below, and for use in evolving new recombinant or chimeric HIV-1 viruses, for use in recursive sequence recombination of nucleic acid segments or other diversity generation methods (e.g., DNA shuffling) reactions, and in methods of the invention, including gene therapy and ex vivo, in vivo, and in vitro applications, as well as other aspects, as described supra and below. In another aspect, recombinant or chimeric HIV-1 viruses are useful as adjuvants to stimulate or augment an immune response to related to an antigen delivered by a retroviral vector (e.g., DNA vaccine) incorporating a recombinant or chimeric HIV-1 polypeptide to a target tissue, cell, or organ. In another aspect, recombinant or chimeric HIV-1 viruses of the invention are useful to produce antibodies that have, e.g., diagnostic and therapeutic and/or prophylactic uses, e.g., related to the activity, distribution, and expression of HIV-1 sequences.

Cells and Animals Infected with Recombinant or Chimeric Virus

In another aspect, the invention provides mammals, including non-human primates, comprising at least one modified, recombinant, or chimeric virus, nucleic acid, and/or polypeptide of the invention. The invention also provides a non-human primate comprising at least one recombinant or chimeric nucleic acid of the invention. In preferred embodiments, the non-human primate is a macaque monkey. Most preferably, the macaque monkey is a pig-tailed macaque monkey.

The invention also provides a macaque monkey comprising at least one modified or chimeric HIV-1 virus of the invention, wherein the at least one modified or chimeric HIV-1 virus replicates for a longer period of time in the macaque monkey than does an HIV-1 virus in the macaque monkey and/or wherein the macaque monkey comprising the modified, recombinant, or chimeric HIV-1 virus exhibits a decrease in a population of CD4+ T cells and/or wherein the macaque monkey comprising at least one modified or chimeric HIV-1 virus exhibits an increase in viremia and/or wherein the macaque monkey comprising at least one modified or chimeric HIV-1 virus exhibits at least one symptom of HIV infection. In one embodiment, the modified or chimeric HIV-1 virus causes prolonged increase in viremia. In a preferred embodiment, the macaque monkey comprising the modified or chimeric HIV-1 virus of the invention exhibits at least one symptom associated with acquired immunodeficiency disease syndrome (AIDS). In another embodiment, the macaque monkey comprising at least one modified or chimeric HIV-1 virus of the invention exhibits at least one symptom of HIV infection that is sustained for a longer period of time than does a macaque monkey comprising an HIV-1 (WT, e.g., DH12) virus.

The invention also provides methods for producing a non-human mammalian cell comprising a modified, recombinant, or chimeric HIV-1 virus that exhibits enhanced replication in a non-human mammalian cell compared to replication of an HIV-1 virus in said non-human mammalian cell. Accordingly, the method entails administering to the non-human mammalian cell a modified, recombinant, or chimeric HIV-1 virus of the invention. In one embodiment, the non-human mammalian cell is a macaque monkey cell. The invention also provides the non-human mammalian cell produced by this method.

The invention further provides a method for producing a non-human mammalian cell comprising a modified, recombinant, or chimeric HIV-1 virus that exhibits enhanced replication in a non-human mammalian cell compared to replication of an HIV-1 virus in said non-human mammalian cell. The method entails administering to the non-human mammalian cell the modified, recombinant, or chimeric HIV-1 virus of the invention.

In addition, the invention provides methods for producing a macaque monkey comprising a modified, recombinant, or chimeric HIV-1 virus that exhibits enhanced replication in a macaque monkey cell compared to replication of an HIV-1 virus in said macaque monkey cell. In one embodiment, the method entails administering to a population of cells of the macaque monkey a modified, recombinant, or chimeric HIV-1 virus comprising at least one recombinant or chimeric HIV-1 nucleic acid of the invention in an amount sufficient to cause an HIV infection in said population of cells of the macaque monkey. In another embodiment, the method entails administering to a population of cells of the macaque monkey at least one modified or chimeric HIV-1 virus of the invention in an amount sufficient to cause an HIV infection in said population of cells of the macaque monkey. In one embodiment of the method of the invention, at least one symptom of HIV infection is produced in the macaque monkey. The invention also provides the macaque monkey produced by the method.

The invention further provides a method for producing a macaque monkey with at least one symptom of HIV infection. The method entails administering to the macaque monkey an evolved viral isolate comprising an evolved HIV-1 virus (described herein), wherein the evolved viral isolate is produced by passaging a first viral isolate at least one time through macaque monkey cells, tissue, or blood, wherein the first viral isolate comprises a recombinant or chimeric HIV-1 virus of the invention comprising one or more recombinant or chimeric HIV-1 nucleic acids of the invention.

Screening Methods

The invention also provides methods of screening for agents that inhibit and/or treat HIV infection. Accordingly, the invention provides a method of screening for an agent that inhibits HIV infection in a non-human primate that entails administering a test agent to a first non-human primate; administering a modified, recombinant, or chimeric HIV-1 virus comprising the recombinant or chimeric HIV-1 nucleic acid the invention to the first non-human primate in an amount sufficient to cause HIV infection; administering this modified, recombinant, or chimeric HIV-1 virus to a second non-human primate in the same amount; monitoring a level of HIV infection and/or an appearance of at least one AIDS-associated symptom in each of the first non-human primate and the second non-human primate, wherein a decrease in the level of HIV infection and/or a delay or absence of the appearance of at least one AIDS-associated symptom in the first non-human primate as compared to the level of HIV infection and/or the appearance of at least one AIDS-associated symptom in the second non-human primate indicates that the test agent inhibits HIV infection. In one embodiment, the first and second non-human primates are macaque monkeys. In a preferred embodiment, the first and second non-human primates are pig-tailed macaque monkeys.

The invention also provides a method for screening for an agent that treats HIV infection that entails providing a first macaque monkey and a second macaque monkey, each of which includes a macaque monkey of the invention; administering a test agent to the first macaque monkey; monitoring a level of HIV infection and/or an appearance of at least one AIDS-associated symptom in each of the first and second macaque monkeys, wherein a decrease in the level of HIV infection and/or a delay or absence of the appearance of at least one AIDS-associated symptom in the first macaque monkey as compared to the level of HIV infection and/or the appearance of at least one AIDS-associated symptom in the second macaque monkey indicates that the test agent treats HIV infection.

The invention further provides a method of screening for an agent that inhibits HIV infection that entails administering a test agent to a first population of primate cells; administering a modified, recombinant, or chimeric HIV-1 virus comprising a recombinant or chimeric HIV-1 nucleic acid of the invention to the first population of primate cells in an amount sufficient to cause HIV infection; administering this modified, recombinant, or chimeric HIV-1 virus to a second population of primate cells in the same amount; monitoring a level of HIV infection in each of the first population of primate cells and the second population of primate cells, wherein a decrease in the level of HIV infection in the first population of primate cells as compared to the level of HIV infection in the second population of primate cells indicates that the test agent inhibits HIV infection. In alternate embodiments, the method is preformed in vitro or ex vivo. In one embodiment, the primate cells are human cells. In another embodiment, the primate cells are macaque monkey cells. In a preferred embodiment, the macaque monkey cells are pig-tailed macaque cells.

In addition the invention provides a method for screening for an agent that treats HIV infection that entails providing a first population of macaque monkey cells and a second population of macaque monkey cells, said first and second population of cells comprising one or more cells of the invention or one or more cells comprising the cell-culture derived progeny of the invention; administering a test agent to the first population of macaque monkey cells; monitoring a level of HIV infection in the first and second populations of macaque monkey cells, wherein a decrease in the level of HIV infection in the first population of macaque monkey cells as compared to the level of HIV infection in the second population of macaque monkey cells indicates that the test agent inhibits HIV infection. In a preferred embodiment, the first and second populations of macaque monkey cells are pig-tailed macaque monkey cells.

### EXAMPLE 1

Materials and Methods

Cell culture. Human and pt mPBMC were purified from heparinized whole blood by centrifugation through Ficoll-Hypaque (Histopaque; Sigma, St. Louis, Mo.) density gradients and either used fresh (for pt mPBMC) or cryopreserved in 90% fetal bovine serum (FBS)/10% dimethylsulfoxide. Pig-tailed macaque PBMC were stimulated with complete media (RPMI 1640 supplemented with 10% FBS, 0.2 mM L-glutamine, 100 U/ml penicillin-streptomycin; all reagents from Gibco BRL, Gaithersburg, Md.) containing 1 ug/ml of phytohemagglutinin (PHA; Sigma) for 3 days and then maintained in the same medium containing 100 U/ml of human recombinant interleukin-2 (IL-2), without PHA. To stimulate human PBMC (huPBMC), recombinant IL-2 was added together with PHA during the initial 3 day treatment.

The human T-cell line MT-4 (Harada 1986, Microbiol. Immunol., 30: 533-544) was maintained in RPMI 1640 supplemented with 10% FBS, glutamine, and antibiotics. Human 293 cells (ATCC) were maintained in Dulbecco's minimal essential medium (DMEM; Gibco BRL, Gaithersburg, Md.) supplemented with 10% FBS and antibiotics.

In vitro infections. MT-4 cells, activated huPBMC (day 5 after PHA stimulation) and pt mPBMC (day 3 after PHA stimulation) were used for in vitro virus infections. Virus inocula were normalized for reverse transcriptase (RT) activity, p24 content or infectious titers as indicated. Infectious titers were determined by performing a limiting dilution infection of MT-4 cells in quadruplicates and calculating the 50% tissue culture infectious dose (TCID₅₀)(Reed 1938, Am. J. Hyg., 27: 493-497). Supernatants from infected PBMC cultures were collected at 2 day or 3 day intervals and monitored for virus associated RT activity or for p24 levels as indicated. RT activity was measured using [32P] dTTP (>400 Ci/mmol; Amersham-Pharmacia, Piscataway, N.J.), as previously described (Willey 1988, J. Virol., 62: 139-147). RT activity was reported as counts of [32P] TTP per minute incorporated in 10 ul (containing 1.67 ul of infected culture supernatant) of the reaction mixture. Levels of HIV-1 p24 in the supernatants were measured using an in-house p24 antigen ELISA standardized with known amounts of p24 antigen. For quantification of SHIV a commercial p27 antigen ELISA (Beckman-Coulter, Miami, Fla.) was used.

Viruses. Infectious full-length proviral clones of 7 clade B [NL4-3 (Adachi 1986, J. Virol., 59: 284-291), HXB2 (Wong-Staal 1985, Nature, 313: 277-284), LAI (Wain-Hobson 1985, Cell, 40: 9-17), JRCSF (O'Brien 1990, Nature, 348: 69-73), YU-2 (Li 1991, J. Virol., 65: 3973-3985), AD-8 (Theodore 1996, AIDS Res. Hum. Retroviruses, 12: 191-194), and DH12 (Shibata 1995, J. Virol., 69: 4453-4462)], 2 clade D [Z2z6 (Srinivasan 1987, Gene, 52: 71-82) and ELI (Alizon 1986, Cell, 46: 63-74)] and the recombinant (clade A/D/I) MAL (Alizon 1986, Cell, 46: 63-74), provided parental sequences for shuffling. A partial clone, UG-15 (unpublished), containing the gag and pol genes of a primary subtype D isolate was also used.

The full-length SHIV molecular clone used as a positive control in this study has been described previously (MD14YE; (Shibata 1997, J. Infect. Dis., 176: 362-373). Briefly, this SHIV comprises the DH12 env, tat, rev, and vpu genes inserted into the backbone of SIVmac239. The premature stop codon within the SIV nef sequence has been repaired and positions 17 and 18 have been changed from RQ to YE. These changes are responsible for the high lymphocyte stimulatory activity of an SIV variant (Du Z. 1995, Cell, 82: 665-674). MD17 is essentially DH12 in which the nef sequence has been replaced by the YE version of the SIVmac239 nef. In the proviral clone of MD17, the 3′ LTR is chimeric; most of the U3 region is derived from SIVmac239, the remainder of U3 as well as the R and the U5 regions are derived from DH12. After reverse transcription, both LTRs will become chimeric. MD17 provided the infectious backbone into which the shuffled sequences were cloned.

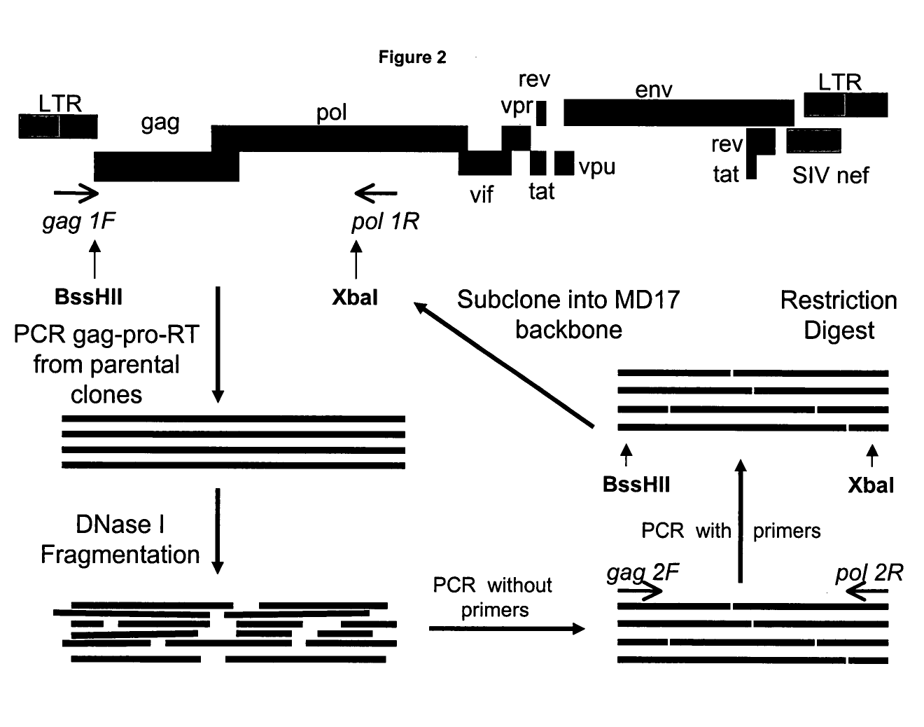

Shuffling of viral sequences and library construction. The shuffling procedure has been described previously (Crameri 1996, Nat. Biotechnol., 14: 315-319) and is shown schematically in FIG. 2. Primer sequences are denoted according to the DH12 sequence nucleotide (nt) positions. Briefly, a 3.5 kb fragment encompassing the entire gag gene, as well as the protease and reverse transcriptase coding sequences of the pol gene, was amplified from each of the 11 parental HIV-1 strains using primer gag 1F (TCT CTC GAC GCA GGA CTC GGC TTG C, nucleotides 680-704) and primer pol 1R (TCA CTA GCC ATT GCT CTC C, nt 4294-4276). PCR products from the 11 parents were mixed together in equimolar amounts and subjected to random fragmentation by DNAseI (Sigma). Fragments ranging from 0.5-1 kb in size were eluted from an agarose gel and reassembled through cycles of denaturation, annealing and extension in the absence of primers. Assembled fragments were then amplified using primer gag 2F (CGG CTT GCT GAA GCG CGC ACG GCA A, nt 697-721) and primer pol 2R (TCT ATT CCA TCT AGA AAT AGT ACT CTC CTG ATT C, nt 4237-4204), digested with BssHII and XbaI and ligated into the MD17 backbone that had been digested with the same enzymes. Ligation mixtures were ethanol-precipitated and electroporated into XL-1 Blue competent cells (Stratagene, La Jolla, Calif.). The entire transformation mixtures were plated on selective agar plates, the colonies were scraped from the plates and plasmid DNA was isolated from the pooled bacteria without further growth in liquid culture. To assess the quality of the generated libraries, 20 sample clones from each library were analyzed for recombination frequency and viability. A rough estimate of recombination frequency was obtained through restriction fragment analysis of the gag-pro-RT region amplified from each clone. Viability was assessed by the ability of the clones to produce infectious virus. This was determined by transfecting single clones into 293 cells (FuGENE 6; Roche, Indianapolis, Ind.) followed by infection of MT-4 cells with the transfection supernatants and monitoring for virus replication by p24 antigen assay.

Library transfection and serial passage. Library DNA was transfected into 293 cells by the calcium phosphate precipitation method (reagents obtained from 5′→3′ Inc., Boulder, Colo.). A mixture comprising all full-length parental HIV-1 molecular clones was transfected in parallel and served as wildtype control. As a positive control for replication in Pt mPBMC, SHIV (MD14YE) was also transfected. Briefly, each 100 mm plate containing 5×106 293 cells was transfected with 30 ug proviral DNA. Sixteen hours after transfection, cells were washed with PBS, and RPMI supplemented with FBS, antibiotics, and IL-2 was added. Supernatants containing virus particles were collected 48 hours later and used to infect freshly stimulated pt mPBMC. To remove input virus PBMC were washed extensively 16 h post infection and were maintained in complete medium supplemented with IL-2 thereafter. Every 2 to 3 days about 90% of the medium was collected for storage at −80 □C and replaced with fresh medium. An aliquot was kept at −20 □C for p24 ELISA or RT activity assay. Two to three weeks after infection, a new passage was initiated by inoculating fresh Pt mPBMC with supernatants collected on the day of peak virus production. Stimulated huPBMC were infected with the pt mPBMC derived virus containing supernatants where indicated. All manipulations of infectious virus were performed under BL3 conditions.

Cloning of full-length HIV-1 and construction of chimeras. The 1B3 virus was propagated in a short-term culture in MT-4 cells. Proviral DNA was PCR-amplified from genomic DNA in two pieces. The 5′ portion of the genome (4.2 kb) was amplified using primers 5′ F (TGG AAG GGA TTT ATT ACA GTG C) and pol 2R. The 3′ portion (5.5 kb) was amplified using primers pol 2F (GAA TCA GGA AAG TAC TAT TTC TAG ATG GAA TAG A) and 3′ R (TGC TAG AGA TTT TCC ACA C, nt 9704-9686). The separate PCR products were then digested at a unique XbaI restriction site (nt 4223) located in the pol gene, ligated together, and subsequently cloned into the pCR-XL-TOPO vector (Invitrogen, Carlsbad, Calif.).

Gag-pro-RT chimeras were generated by exchanging the BssHII (nt 709) to XbaI (nt 4223) fragments between MD17 and 1B3 clone 1.4. Exchange of the region spanning integrase through envelope was accomplished by swapping the pieces between the XbaI site (nt 4223) and the SalI site (nt 8465). For construction of the nef-LTR chimeras, the pieces between the SalI site (nt 8465) and a BamHI site in the vector multiple cloning site were exchanged between 1B3 clone 1.4 and MD17 or between 1B3 clone 1.4 and DH12. The correct composition of each chimera was confirmed by sequence analysis. All chimeric clones were tested for viability by infection of human cells prior to use in the macaque tropism experiments.

Results

Parental HIV-1 Strains Replicate Poorly on pt mPBMC.

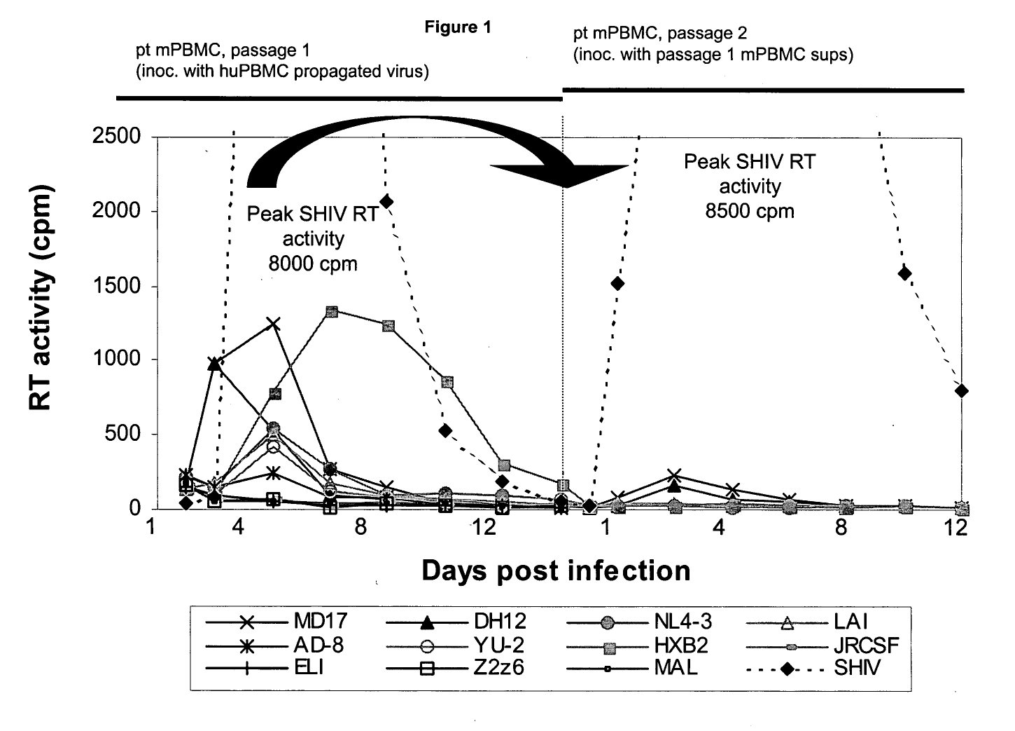

We evaluated the 10 full-length, parental HIV-1 clones for their ability to replicate on pt mPBMC. These viruses were first propagated by short-term passage on huPBMC. RT-normalized amounts of the viruses were then used to infect fresh pt mPBMC. FIG. 1 shows that the HIV-1 strains replicated poorly or did not replicate at all, consistent with previous reports (Agy 1992, Science, 257: 103-106; Frumkin 1993, Virology, 195: 422-431; Gartner 1994, J. Med. Primatol., 23: 155-163; Otten 1994, AIDS, 8: 297-306; Kimball 1998, J. Med. Primatol., 27: 99-103). HXB2, DH12 and its SIV nef-containing derivative MD17, produced the highest RT levels although these were still less than 20% that of SHIV positive control. Virus production peaked at day 4 post-infection and sharply decreased thereafter.

We attempted to passage the viruses by inoculating fresh pt mPBMC with peak RT supernatants from the first passage. Only DH12 and MD17 survived the transfer and replicated slightly above background levels. A third passage resulted in no detectable viral production. No infectious virus could be rescued from this passage even by co-cultivation with permissive human cells (data not shown). In contrast to the HIV-1 strains tested, SHIV showed high and sustainable replication on pt mPBMC throughout several passages. Thus, our results confirm previous studies reporting that HIV-1 replicates poorly and cannot sustain a continuous infection in pig-tailed macaque cells (Gartner 1994, J. Med. Primatol., 23: 155-163; Kimball 1998, J. Med. Primatol., 27: 99-103).

Generating Infectious HIV-1 Libraries Containing Shuffled gag-pro-RT Sequences.

Our approach comprised shuffling a 3.5 kb region between conserved BssHII and XbaI sites, which encompassed the entire gag, protease and reverse transcriptase sequences. Determinants that restrict macaque cell tropism have been mapped to this region (Shibata 1995, J. Gen. Virol., 76: 2723-2730). We reasoned that recursive sequence recombination or other diversity generation methods (e.g., DNA shuffling) of different HIV-1 sequences in this region might generate favorable recombinants that would alleviate this restriction. Ten full-length and one partial HIV-1 clone from several clades provided the starting sequence diversity for shuffling. The homology within the shuffled region for these parent sequences ranged from 90% to 99%.

FIG. 2 outlines the scheme for generating shuffled gag-pro-RT libraries. The shuffled sequences were cloned into an infectious MD17 backbone. MD17 is a derivative of the dualtropic DH12 strain (Shibata 1995, J. Virol., 69: 4453-4462) and except for nef, consists entirely of HIV-1 genes. In the MD17 nucleotide sequence, the HIV-1 nef nucleotide sequence has been replaced with the SIV-derived nef nucleotide sequence containing the ‘YE’ mutations. This nef mutant induces strong proliferation of resting mPBMC in vitro and a highly pathogenic acute infection in vivo (Du Z. 1995, Cell, 82: 665-674). The presence of the SIV “YE” nef alone does not confer a significant replicative advantage to MD17; like DH12, MD17 still replicated poorly in pt mPBMC and could not sustain a productive infection (FIG. 1).

We generated 4 shuffled HIV-1 libraries (1B3, 2B3, 2A3, 2A6), each containing between 4×10⁴ and 8×10⁴ clones. Restriction fragment analysis using DraI and HinfI of several independent clones from each library revealed that 79% to 100% of the clones were recombinant within the shuffled region. Between 25% and 45% of the clones produced virus that productively infected human MT-4 cells (data not shown). This served as a measure of the viability of the libraries.

Emergence of an Improved Variant After Serial Passaging of Shuffled HIV-1 Libraries in pt mPBMC.

Transient transfection of the 4 shuffled proviral libraries and a control mixture of the parental clones into 293 cells produced the virus pools that were used to infect separate pt mPBMC cultures. All 4 libraries and the parental mixture replicated poorly in this first passage (data not shown). None of these viral cultures survived a second passage in fresh pt mPBMC. This result was not surprising, since clones exhibiting an augmented replication phenotype might be expected to be a minor component of each library. Also, we reasoned that additional mutations may be required to manifest incremental advantages conferred by shuffling. In a previous study, in which we applied DNA shuffling to evolve MLV for a new cell tropism (Soong 2000, Nat. Genetics, 25: 436-439), we selected the shuffled library for 4 passages before the new activity became evident. To enable the enrichment process to proceed in this previous study, the selection cultures contained a small number of semi-permissive cells mixed with a majority of target cells.

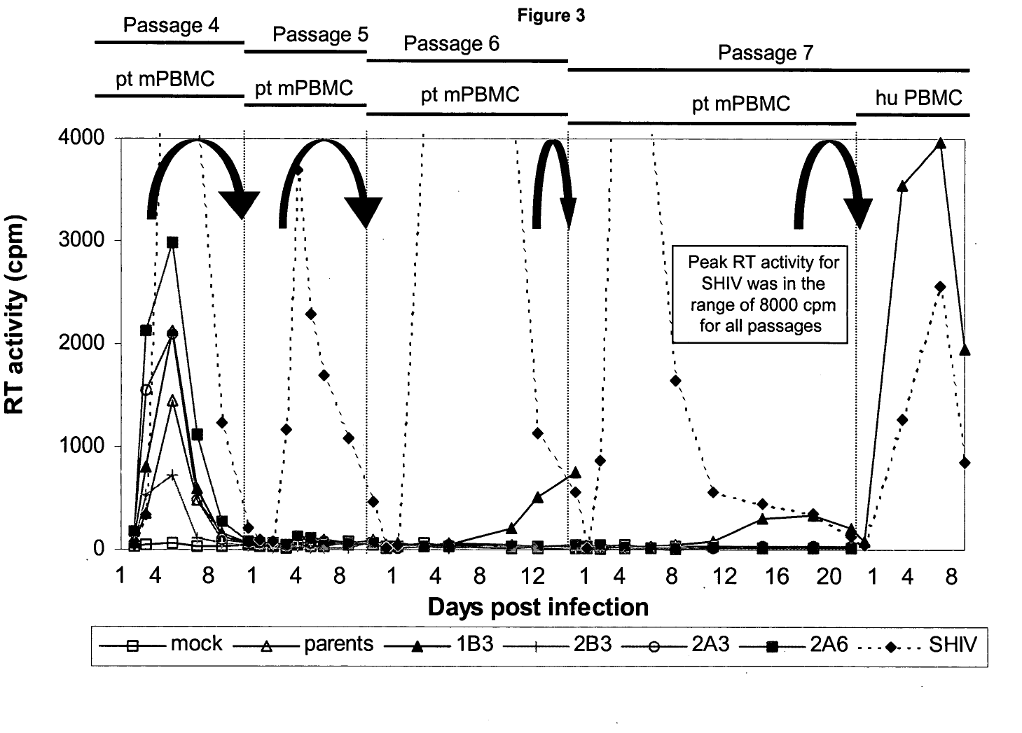

We thus used a variation of this strategy here by inoculating permissive huPBMC with viral supernatants from infected pt mPBMC to rescue and amplify progeny viruses. The amplified viruses were then used to initiate the next round of infection in fresh pt mPBMC. We performed 3 cycles of this alternating passaging regime; all 4 libraries as well as the parental culture recovered and replicated to high levels when huPBMC were inoculated in the first 3 passages (data not shown). An infection of a 4th passage of pt mPBMC was initiated using viruses amplified in huPBMC. From this point (passage 5 and above), we increased the stringency of the selection by directly infecting fresh pt mPBMC with viral supernatants derived from the previous pt mPBMC passage, without an intervening huPBMC amplification step. Among the HIV-1 cultures, only the 1B3 and 2A6 libraries showed marginal levels (70 cpm above background of 40 cpm) of virus replication during passage 5 (FIG. 3). Another direct infection of fresh pt mPBMC was performed for passage 6 where RT levels increased only in the 1B3 culture after a 10 day delay. This pattern was repeated in passage 7.

After 4 successive passages (passages 4-7) in pt mPBMC, we attempted to rescue viruses from the cultures using huPBMC. This was successful only with the 1B3 library and SHIV control cultures. In contrast, virus could not be recovered from the other 3 libraries and the parental HIV-1 cultures at passage 6 and passage 7, showing that these viral populations had not survived the stringent selection. Restriction digests of proviral gag-pro-RT sequences, amplified by PCR from the genomic DNA of 1B3 infected huPBMC, revealed a pattern distinct from any of the parental clones (data not shown). This suggests that a dominant, recombinant species had emerged from the 1B3 library.

Viral Variant 1B3 Shows Improved pt mPBMC Replication.

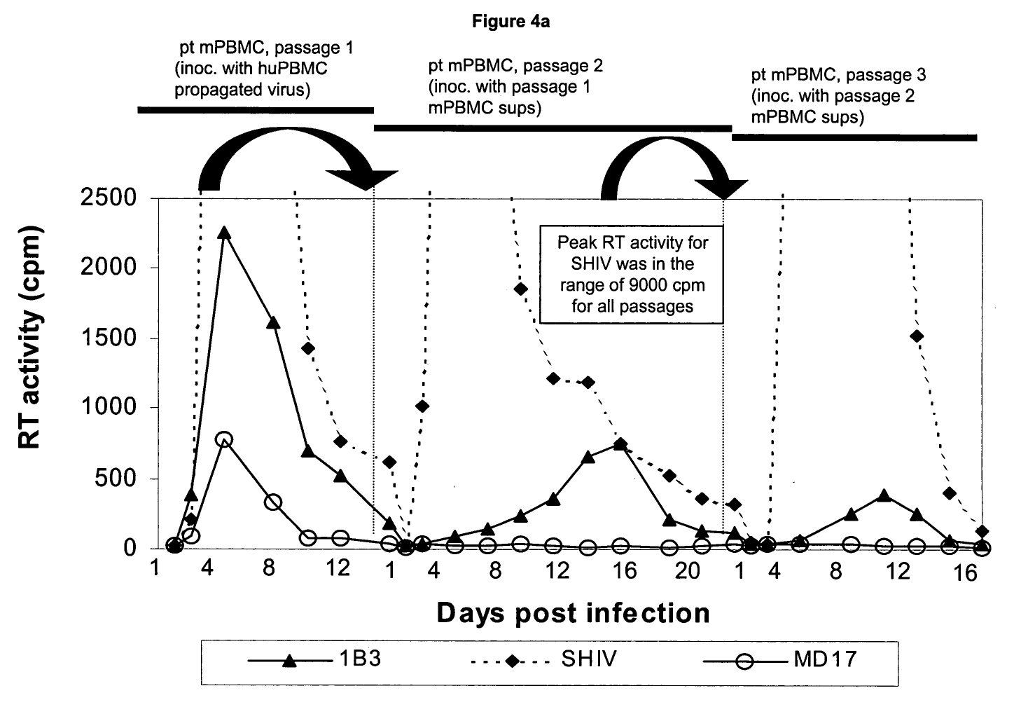

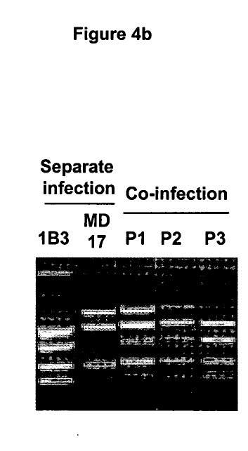

Next, we directly compared the replication kinetics of the evolved 1B3 virus to that of MD17, the parental strain that replicated best in pt mPBMC. SHIV was included as a positive control. The viruses were propagated by short-term culture in huPBMC, normalized for RT activity and used to infect pt mPBMC. Virus production peaked on day 4 and declined thereafter (FIG. 4a). The peak RT activity of 1B3 was 3 fold higher than MD17 and 4.5 fold lower than SHIV. We performed a second pt mPBMC passage using day 4 supernatants from passage 1 pt mPBMC cultures normalized for RT activity. While no virus replication was detected in the MD17 infected cells, RT activity increased in the 1B3 culture and peaked on day 13. A 3rd passage of all 3 viruses on pt mPBMC inoculated with passage 2 derived supernatants resulted in similar patterns of replication. Several attempts to rescue MD17 from passage 3 derived supernatants with permissive huPBMC failed. In contrast, infectious virus was readily recovered from 3rd passage 1B3 infected pt mPBMC. 1B3 could continue to replicate similarly through at least 2 more consecutive pt mPBMC passages (passage 5) before the experiment was terminated (data not shown). These data demonstrate that the evolved 1B3 virus has adapted to replicate better in pt mPBMC than the best HIV-1 parent, MD17. It is still not as fit as SHIV, which contains the entire gag-pol region from SIV. Further evidence of the improved fitness of 1B3 was obtained by directly competing 1B3 and MD17 in a co-infection experiment. For the first passage, pt mPBMC were co-infected with equal amounts (normalized for infectivity) of both viruses.

Infections of fresh pt mPBMC were continued for 2 more passages. Virus from each passage was recovered using huPBMC. Proviral sequences amplified from these infected huPBMC were then analyzed to monitor the existing viral population at each passage. The distinctive restriction patterns of the gag-pro-RT regions of 1B3 and MD17 allowed us to ascertain the composition of the existing population. FIG. 4b shows the progress of the competition. At passage 1 and 2, both viruses were present but by passage 3, 1B3 had essentially gained complete dominance.

Molecular Cloning and Sequence Analysis of 1B3.

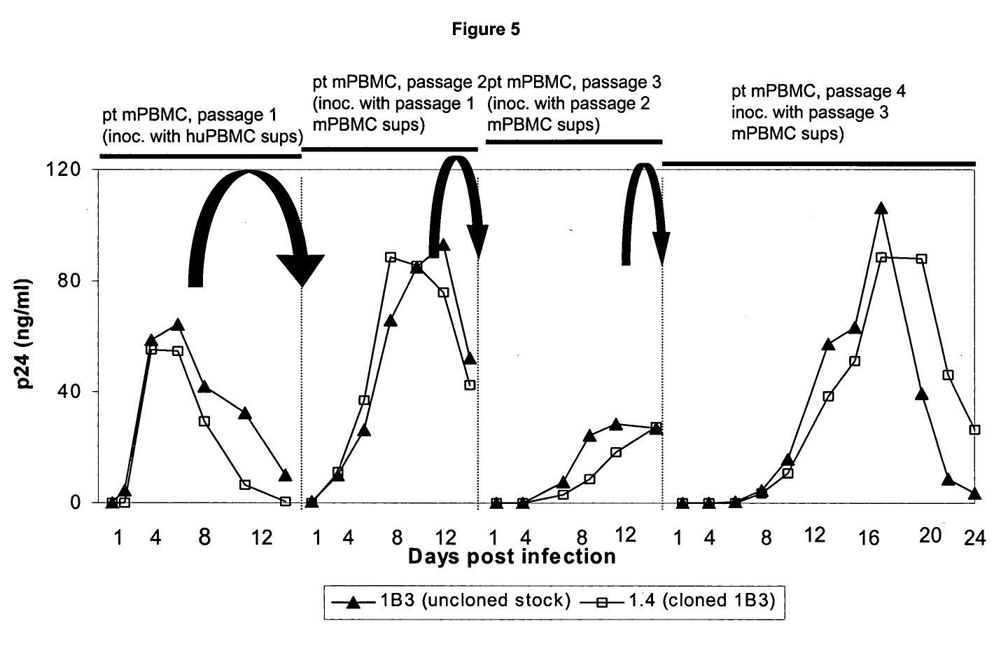

For generation of full-length molecular clones of the 1B3 virus the proviral genome was amplified from infected MT-4 cells in two pieces, ligated together using a unique XbaI site and then cloned into a TA cloning vector. Of 100 clones tested, 13 generated virus that could productively infect human MT-4 cells; of these, 5 were able to persist through several consecutive passages in pt PBMC. In a side-by-side comparison, the replication kinetics of the improved clone 1.4 was indistinguishable from that of the uncloned 1B3 virus. (FIG. 5) Similar to the uncloned 1B3 virus, clone 1.4 also out-competed the parental MD17 in a co-infection of pt mPBMC (data not shown). Thus, clone 1.4 possessed the improved replication phenotype observed for the original evolved 1B3 virus.

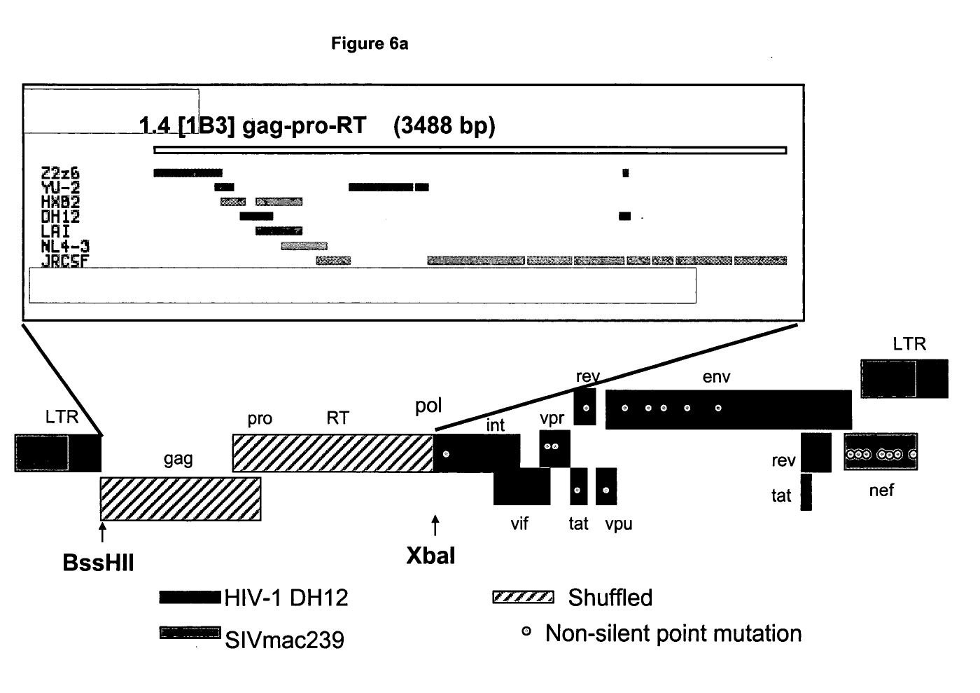

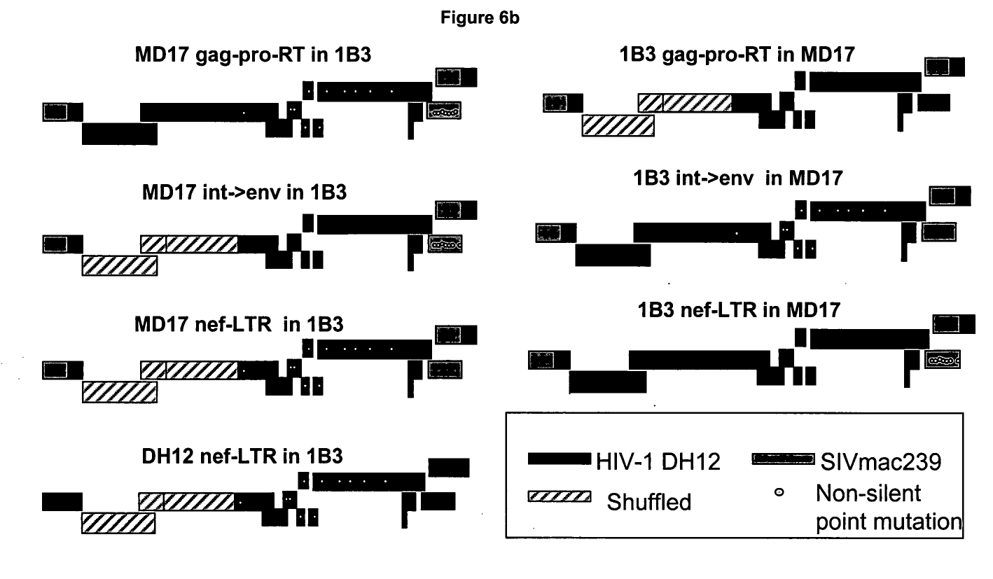

We sequenced the entire genomes of clone 1.4 (sequence will be deposited in Genbank with accession number listed here at the proof stage) and 3 other clones that also exhibited the improved pt mPBMC replication phenotype. These clones all shared a similar structure for the shuffled gag-pro-RT region and several similar mutations in the rest of the genome. The composition of the shuffled region and non-silent point mutations for clone 1.4 are shown diagrammatically in FIG. 6a. Analysis of the shuffled gag-pro-RT region revealed that sequences of at least 7 parental HIV-1 strains recombined to generate the gag sequence of 1B3. The protease and RT coding region were most likely derived from the JRCSF parent.

In the non-shuffled regions of the genome, we observed several amino acid changes relative to the original MD17 or DH12 (SEQ ID NO:40; GenBank Acc. No. AF069140) sequence that were common to all the sequenced clones. These are denoted from the start of each protein. In the integrase coding region of Pol, a Ser to Asn change (position 730) was consistently found. Changes resulting in the substitutions of Glu to Lys at position 21, and of Gly to Glu at position 51, were observed in the vpr sequence. Tat contained an Arg to Lys substitution at position 53; rev contained a Glu to Lys substitution at position 7, and in vpu, the Arg at position 49 was changed to Lys. Several similar amino acid changes were also found throughout the gp120 coding portion of env (Met to Ile at positions 150 and 468, Glu to Lys at positions 320 and 346, Val to Ile at position 358). There were also a number of consistent changes in the SIVmac239 derived nef sequence. These were: Asp to Asn at position 15, Arg to Lys at position 30 and 245, and Glu to Lys at positions 36, 75 and 92. Clone 1.4 and another improved clone contained an additional Glu to Lys mutation at position 147.

Contribution of Sequence Changes to Improved pt mPBMC Tropism

The changes in the sequences of the 1B3-derived clones from the original MD17 parent can be attributed to shuffling in the gag-pro-RT region as well as adaptive changes in the rest of the genome. To elucidate the relative contributions of these regions to the improved phenotype, we generated six reciprocal chimeras (FIG. 6b) between clone 1.4 and the MD17 parent and assayed their ability to replicate in pt mPBMC using normalized amounts of input virus in each experiment.

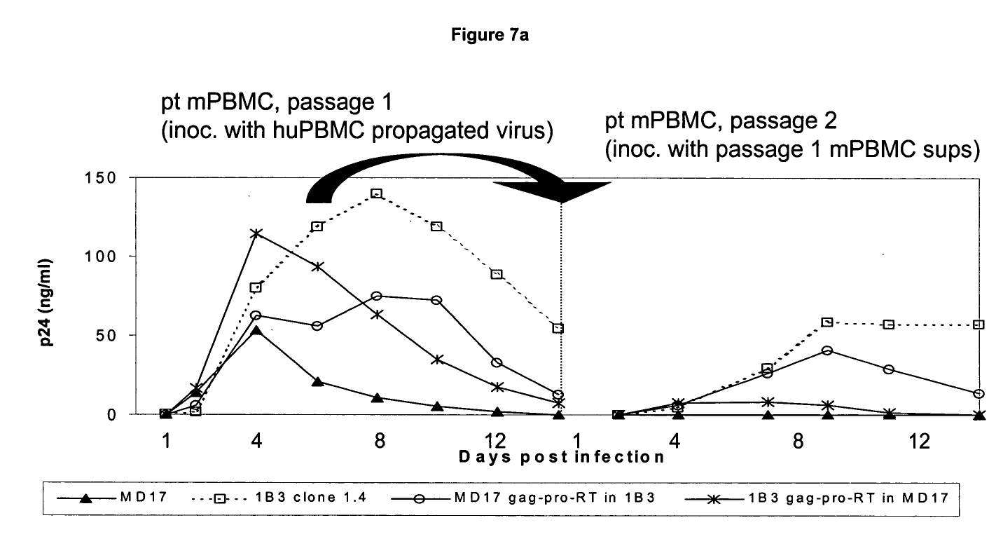

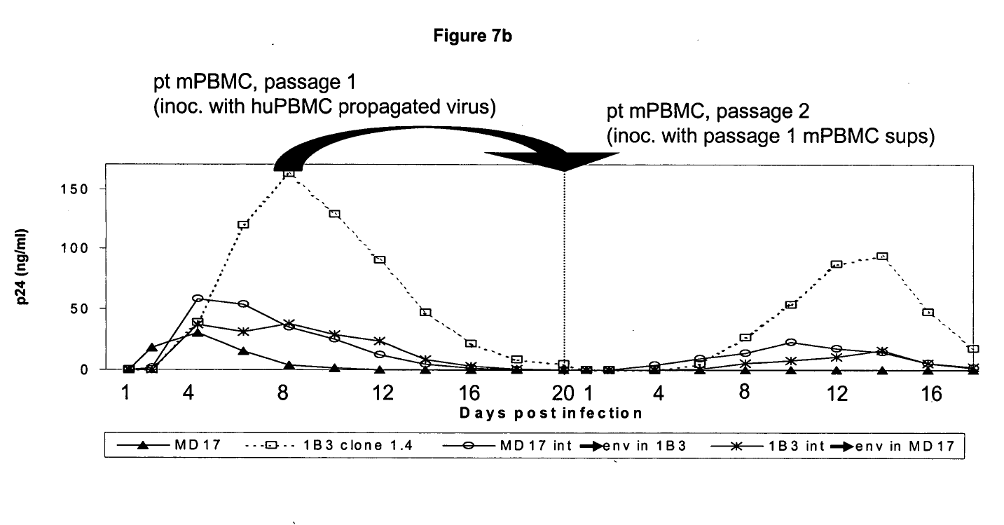

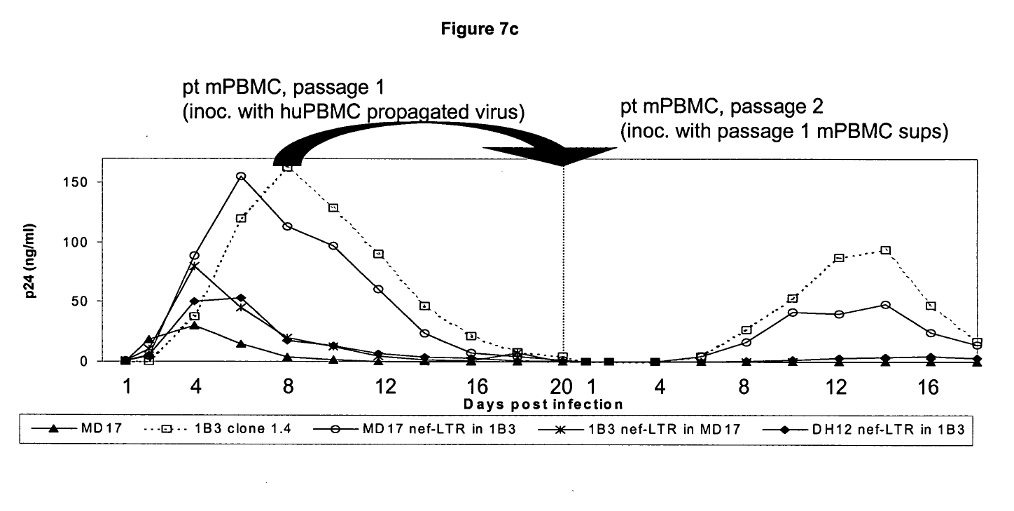

First, when the shuffled 1B3 gag-pro-RT region from clone 1.4 was transplanted into the MD17 backbone, improved replication as assayed by p24 production, compared to the parent MD17 was observed (FIG. 7a). Conversely, when the shuffled region was replaced by MD17 gag-pro-RT sequences, p24 levels decreased compared to clone 1.4.

These observations suggest that the shuffled gag-pro-RT region did impart a replicative advantage, although it was by itself insufficient to confer the full, improved phenotype observed for 1B3.

Next, the 4.2 kb fragment (int→env) containing integrase, envelope as well as the regulatory genes vif, vpr, vpu, tat and rev, was exchanged between 1B3 clone 1.4 and the MD17 parent. This region was not shuffled, but had acquired several adaptive changes. Transplanting 1B3 int→env sequences into MD17 increased p24 production and persistence compared to MD17 (FIG. 7b). Replacing this region in clone 1.4 with MD17 sequences decreased replicative ability. Thus, adaptive changes in this non-shuffled region contributed to the augmented replication phenotype but were not sufficient to confer the full improvement observed for 1B3.

Finally we examined whether changes in the unshuffled nef-LTR region had any effect on pt mPBMC replication (FIG. 7c). The chimera containing 1B3 nef-LTR in the MD17 backbone was slightly improved in the first passage, but only replicated marginally in the second passage (<1 ng/ml). The reciprocal chimera, harboring MD17 nef-LTR in clone 1.4 replicated similarly to clone 1.4 at passage 1, and at half the levels of clone 1.4 during the second passage. Collectively, the data suggest that the changes in nef-LTR also confer improvements. Replacing the SIV nef-LTR sequences in clone 1.4 with HIV-1 nef-LTR sequences resulted in a chimera (DH12 nef-LTR in 1.4) that lost the improved phenotype and that replicated similarly to MD17 (FIG. 7c). This suggested that SIV nef sequences are required for the beneficial changes in the other regions to be manifested.

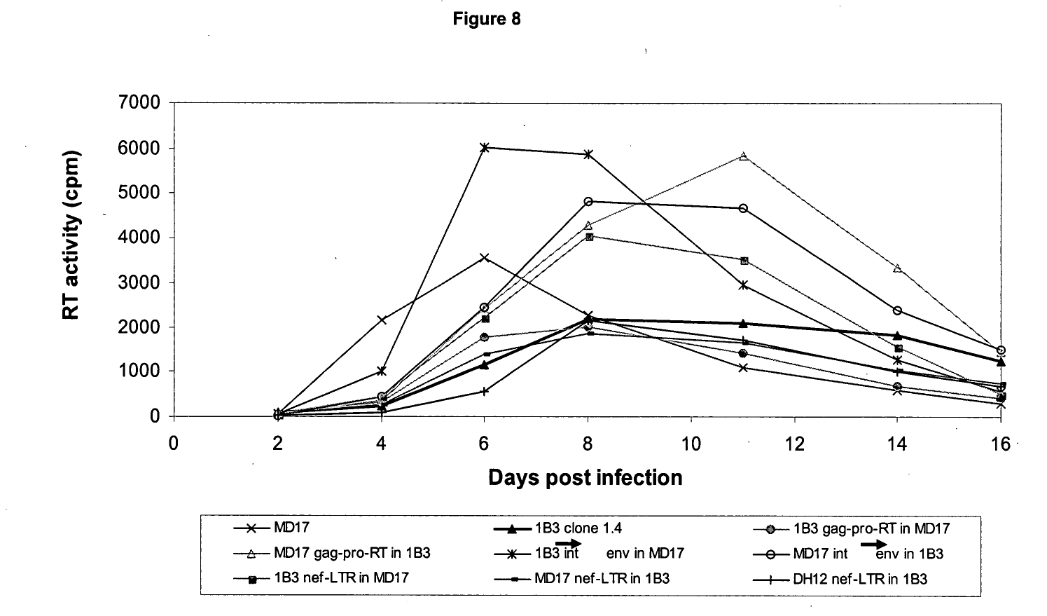

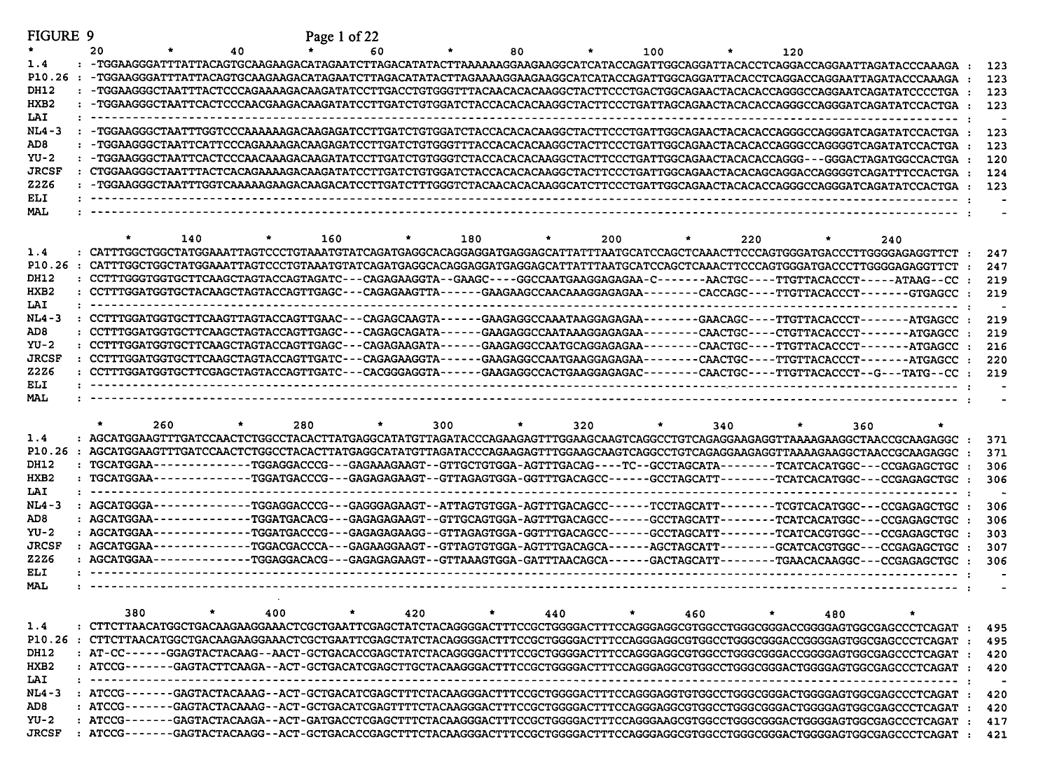

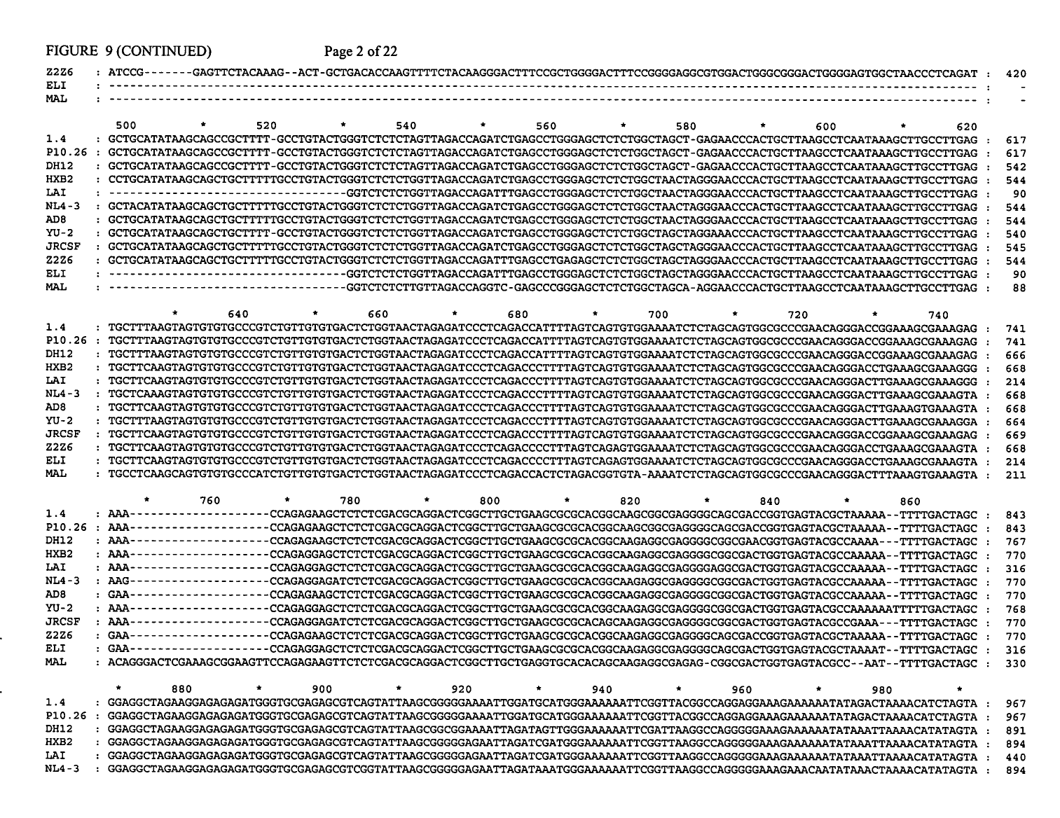

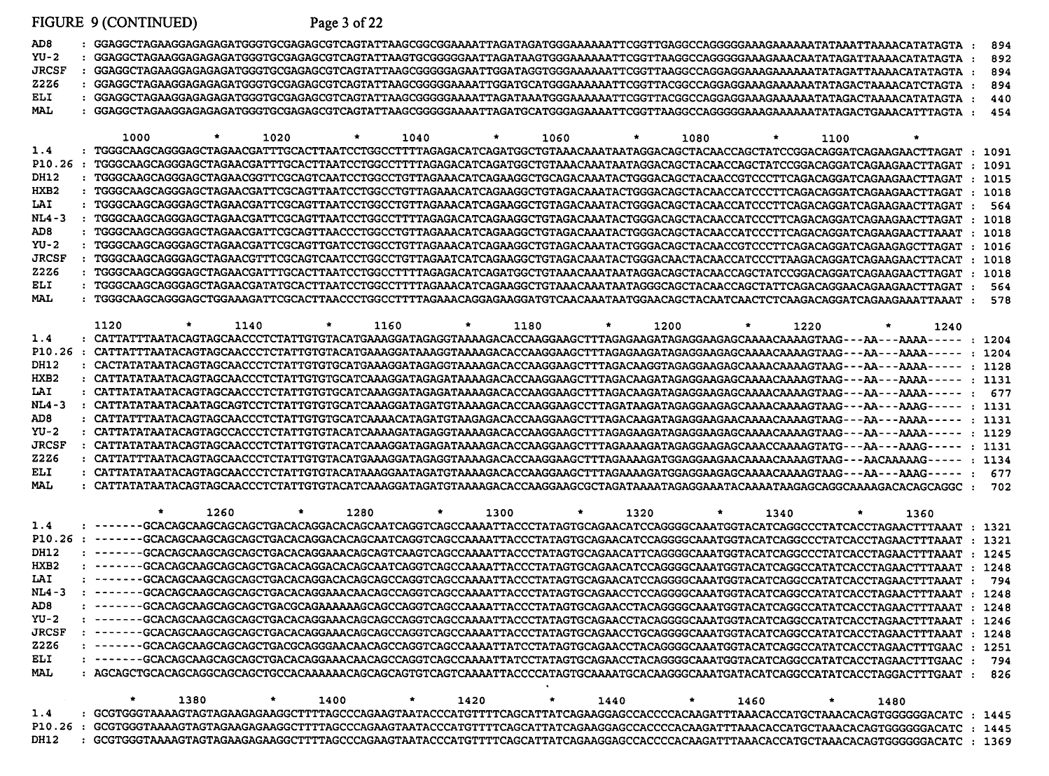

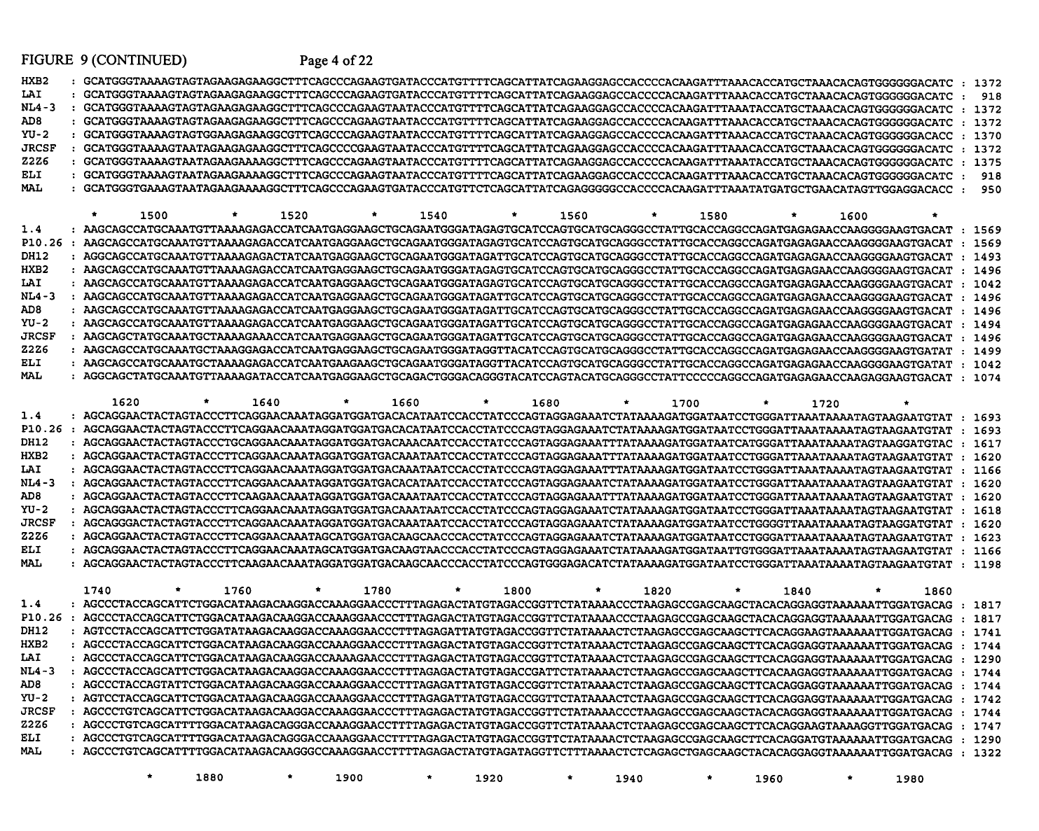

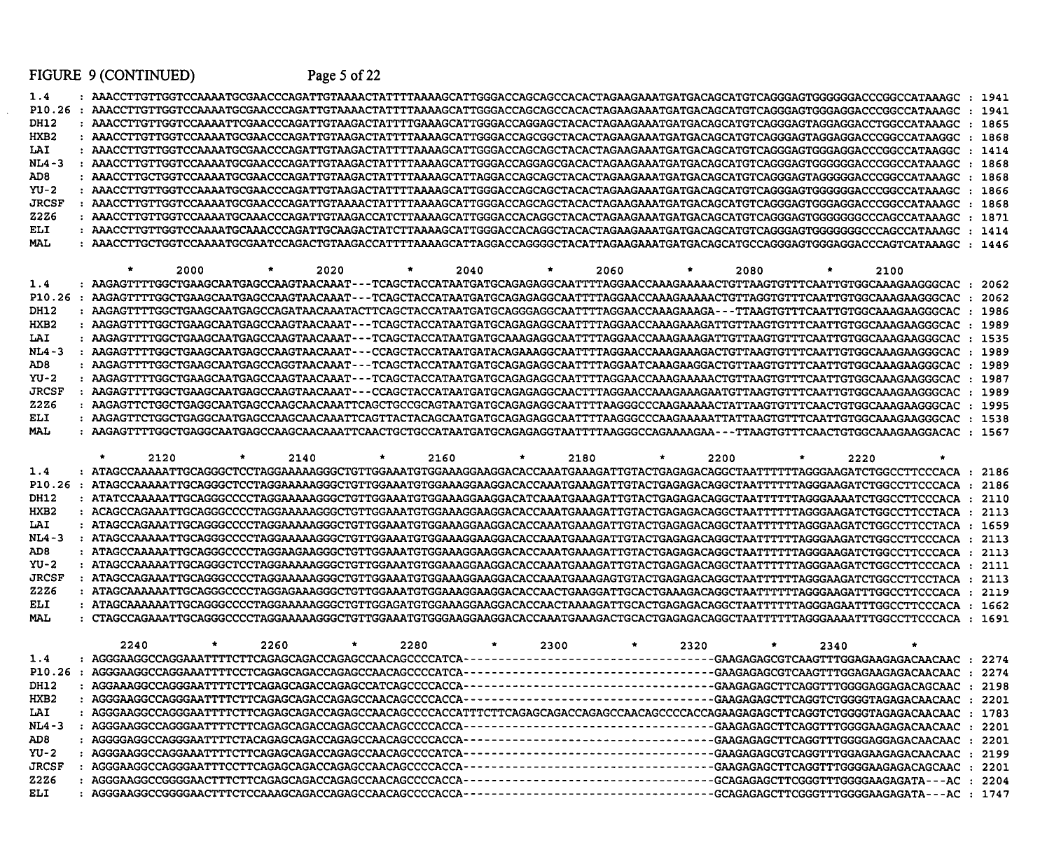

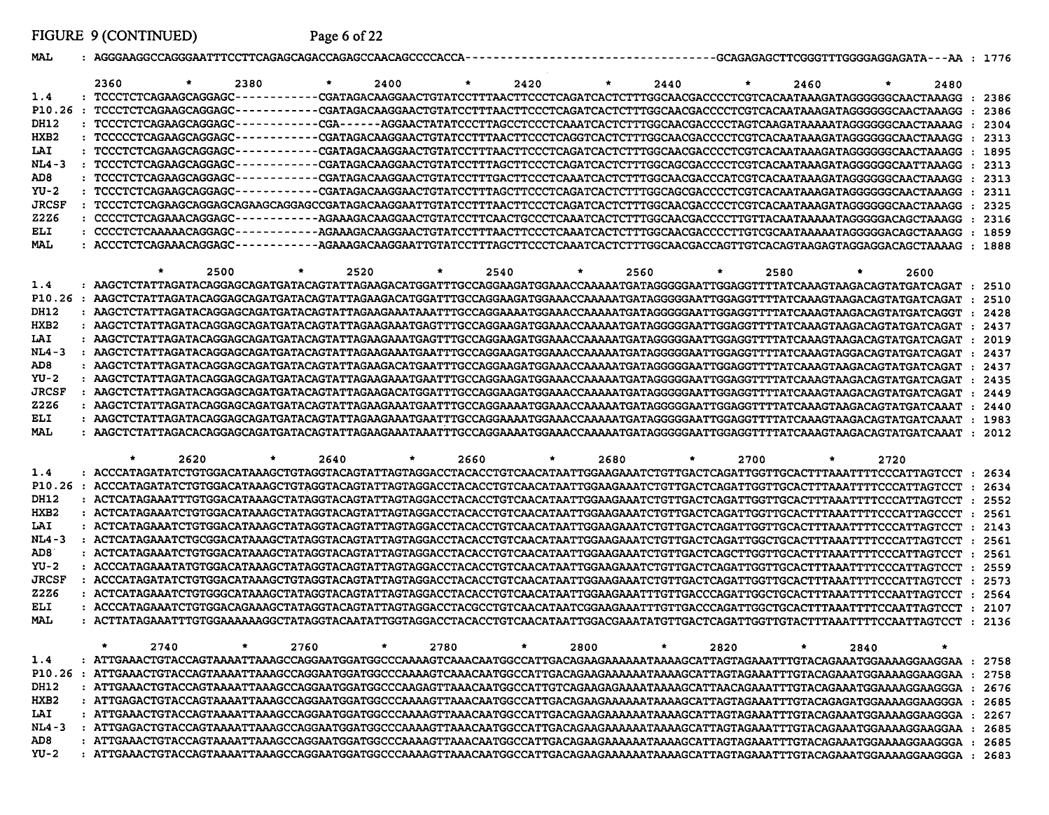

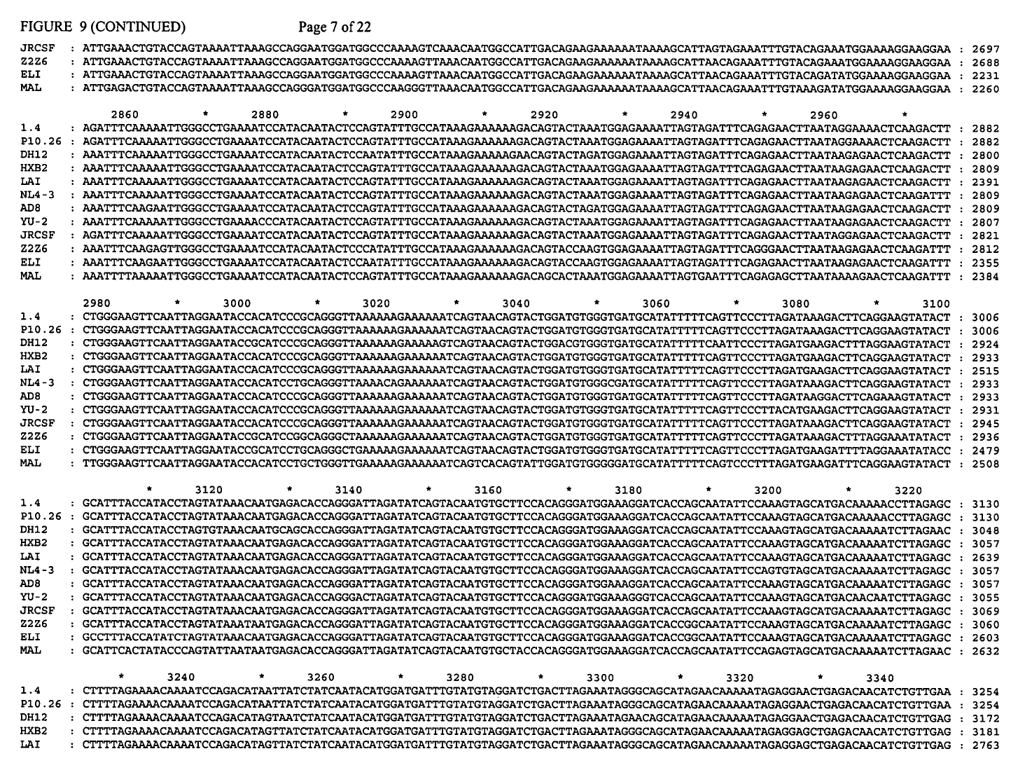

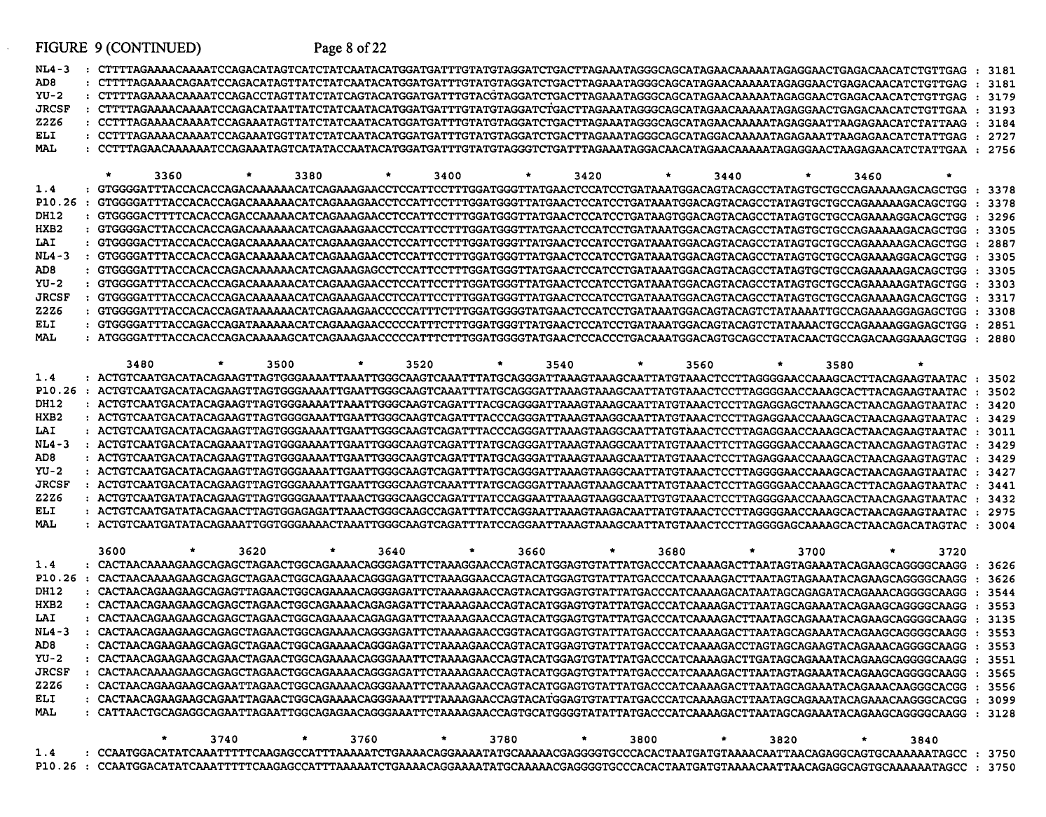

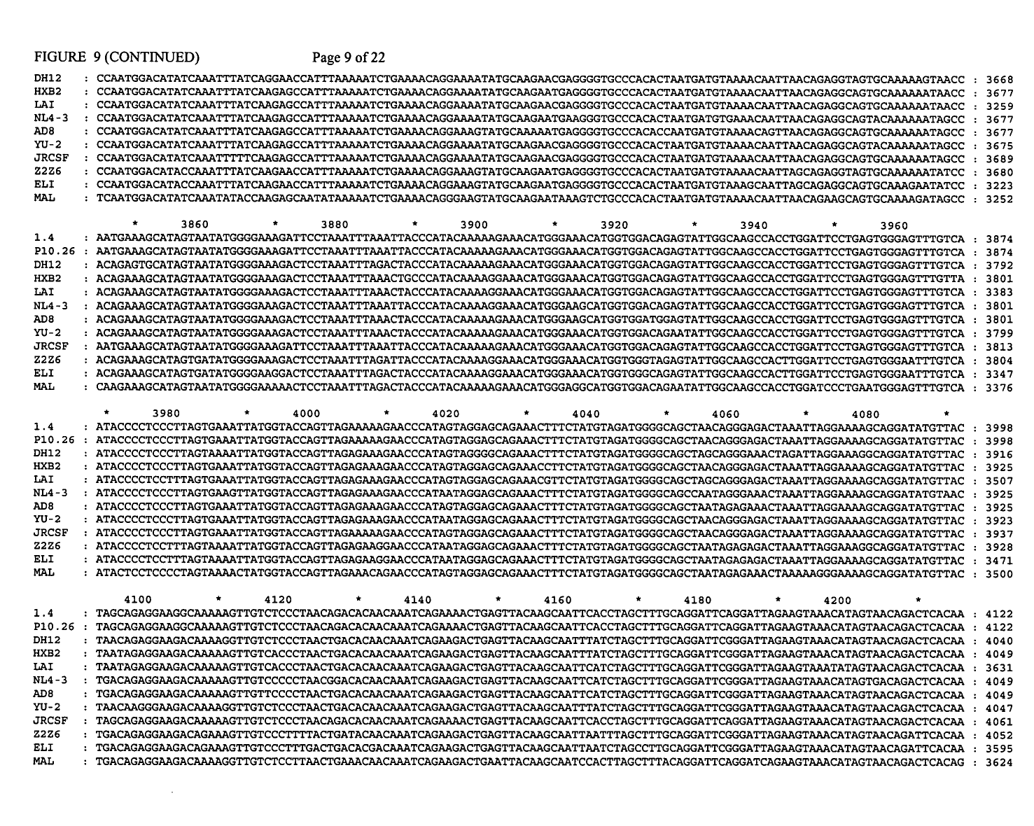

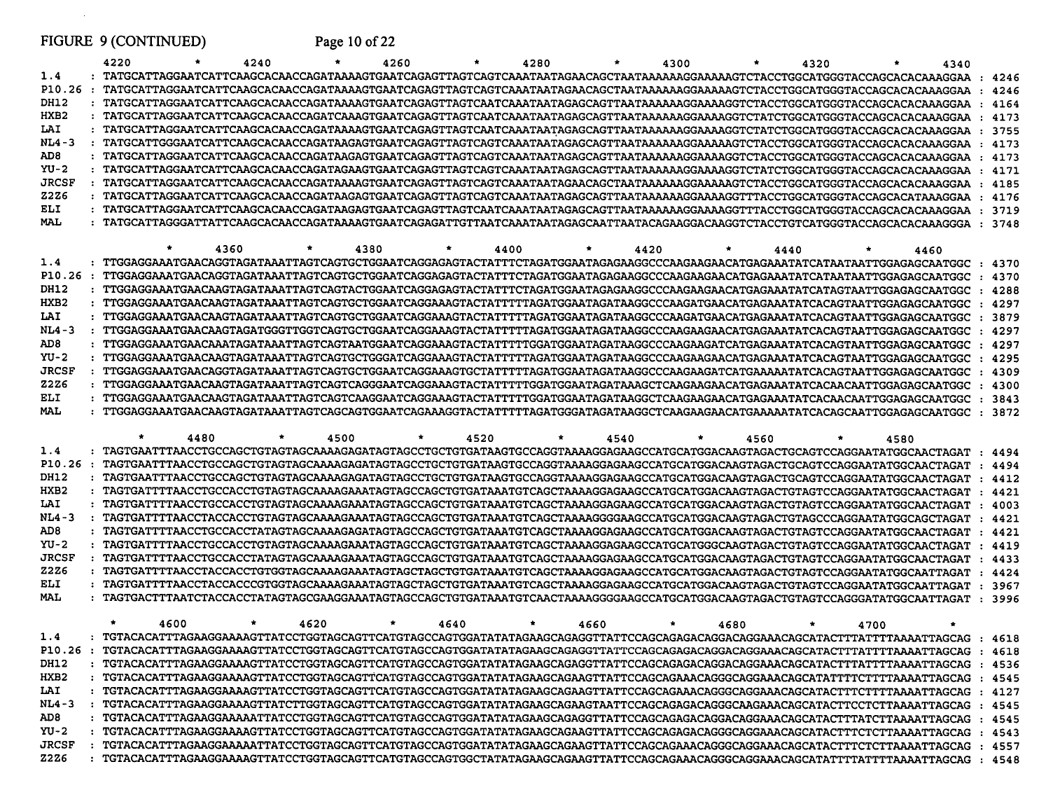

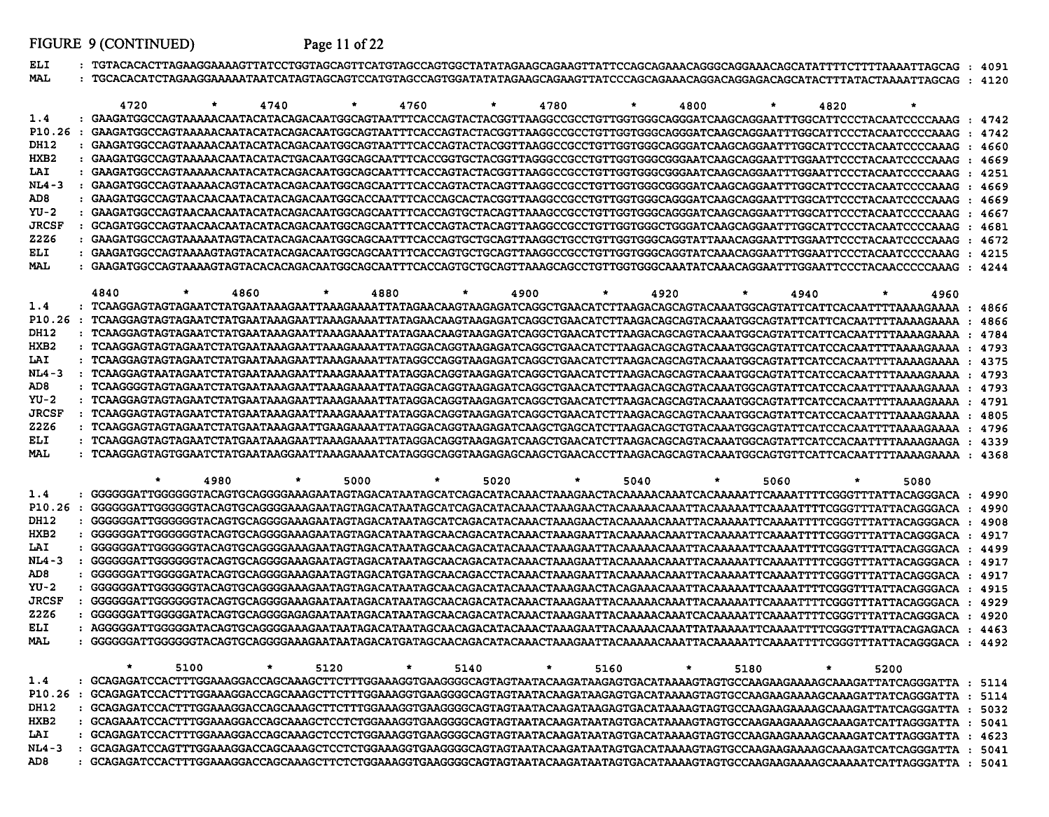

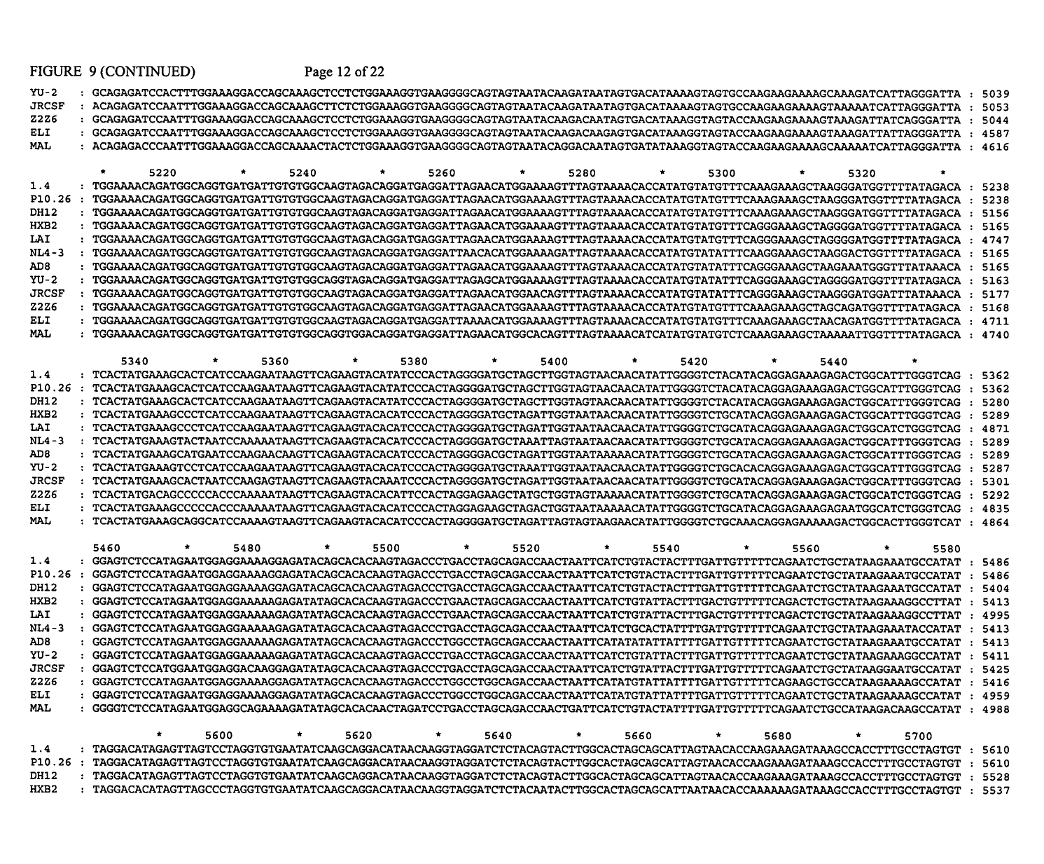

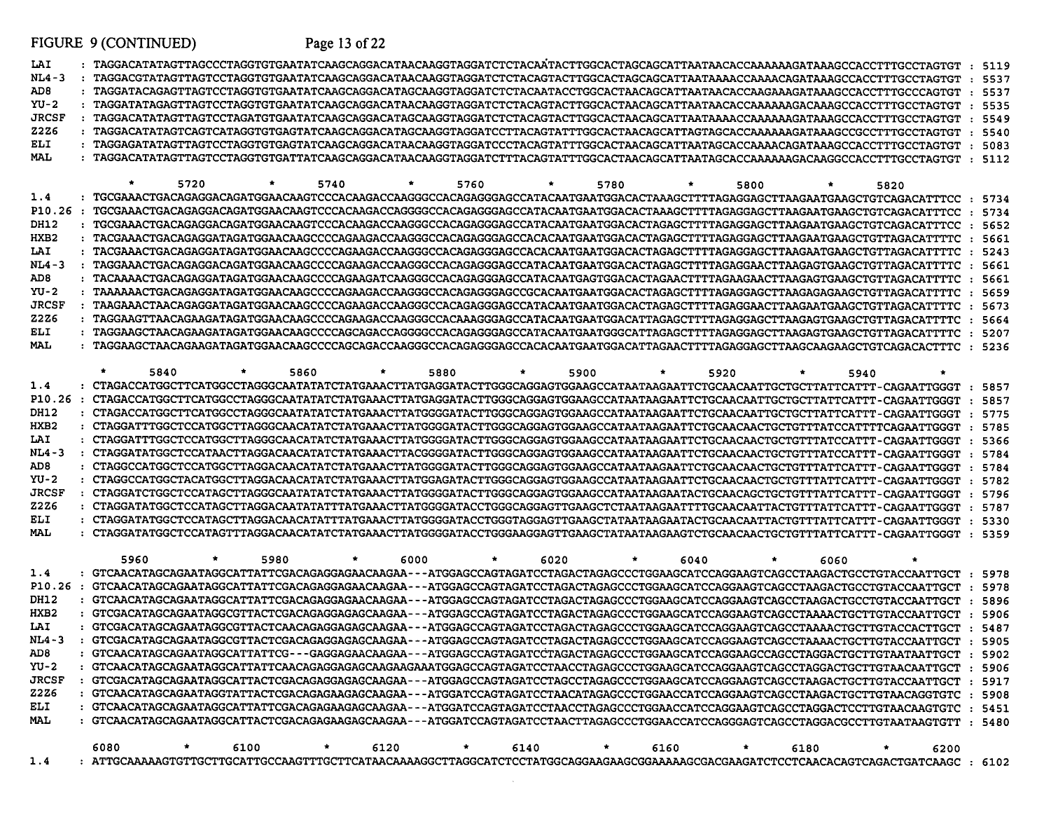

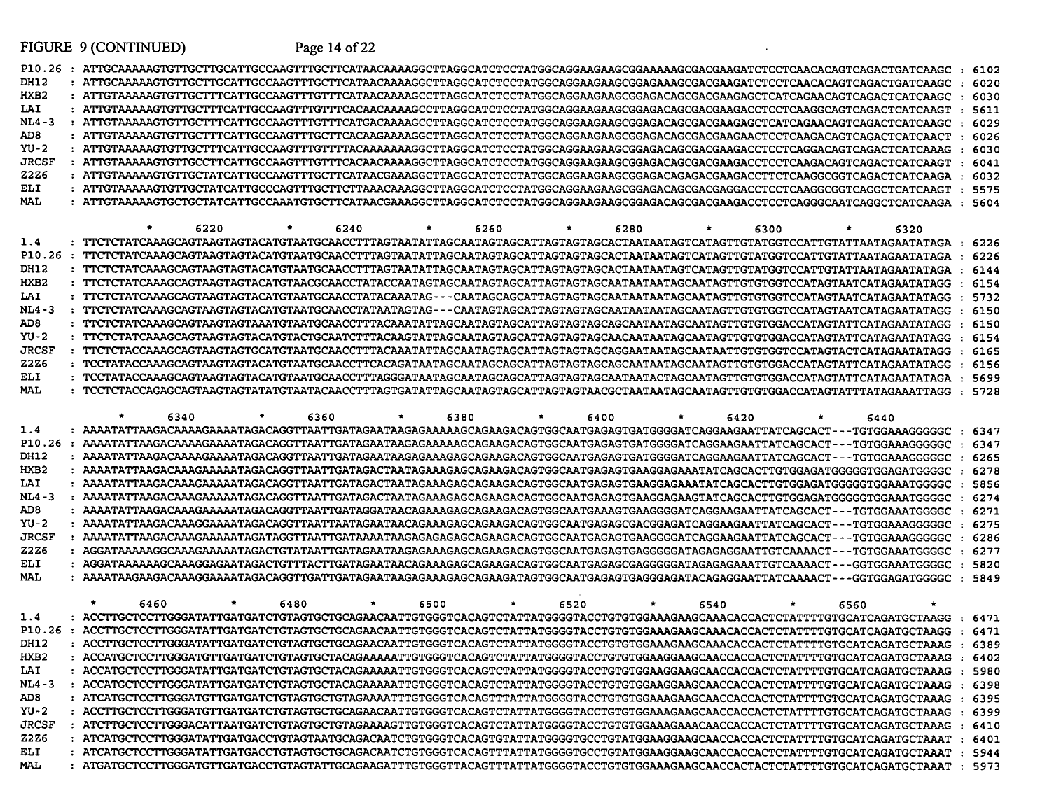

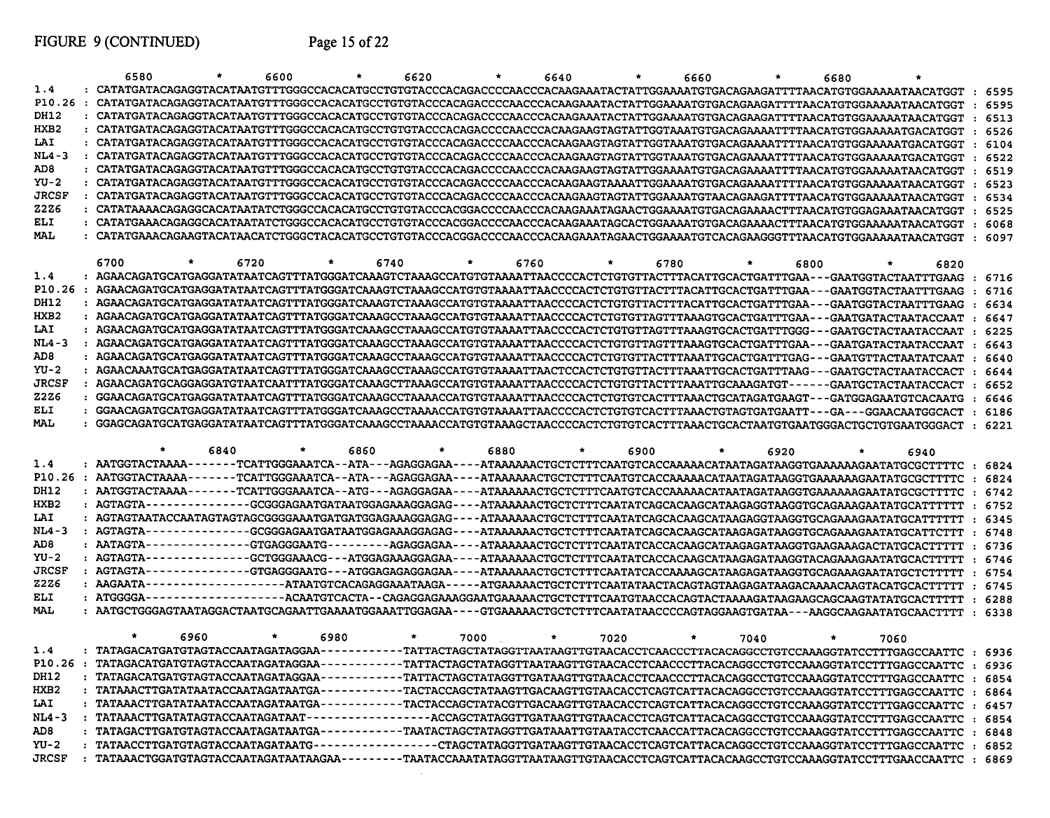

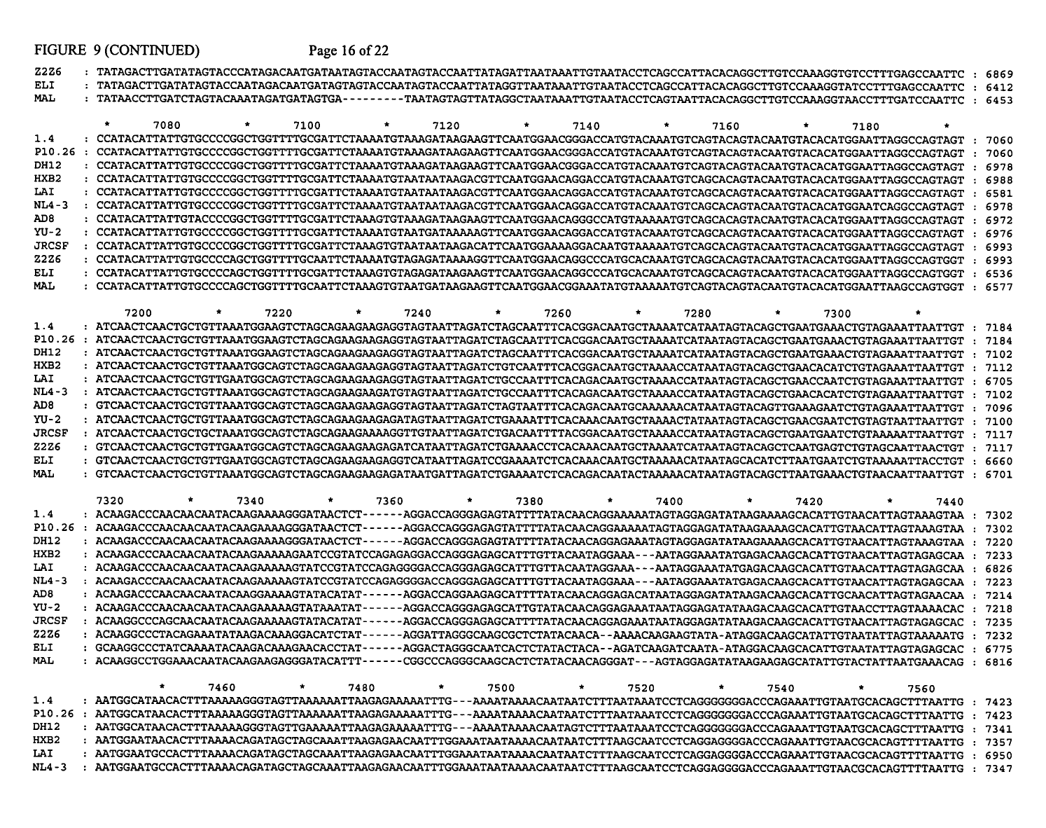

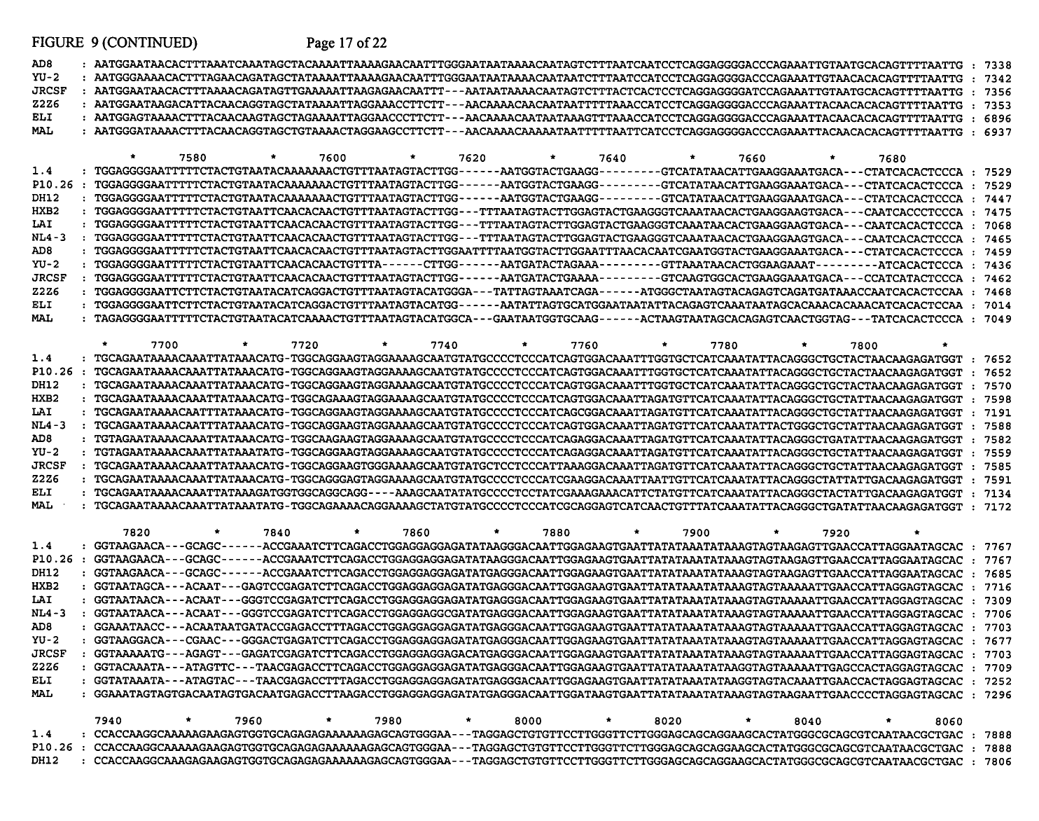

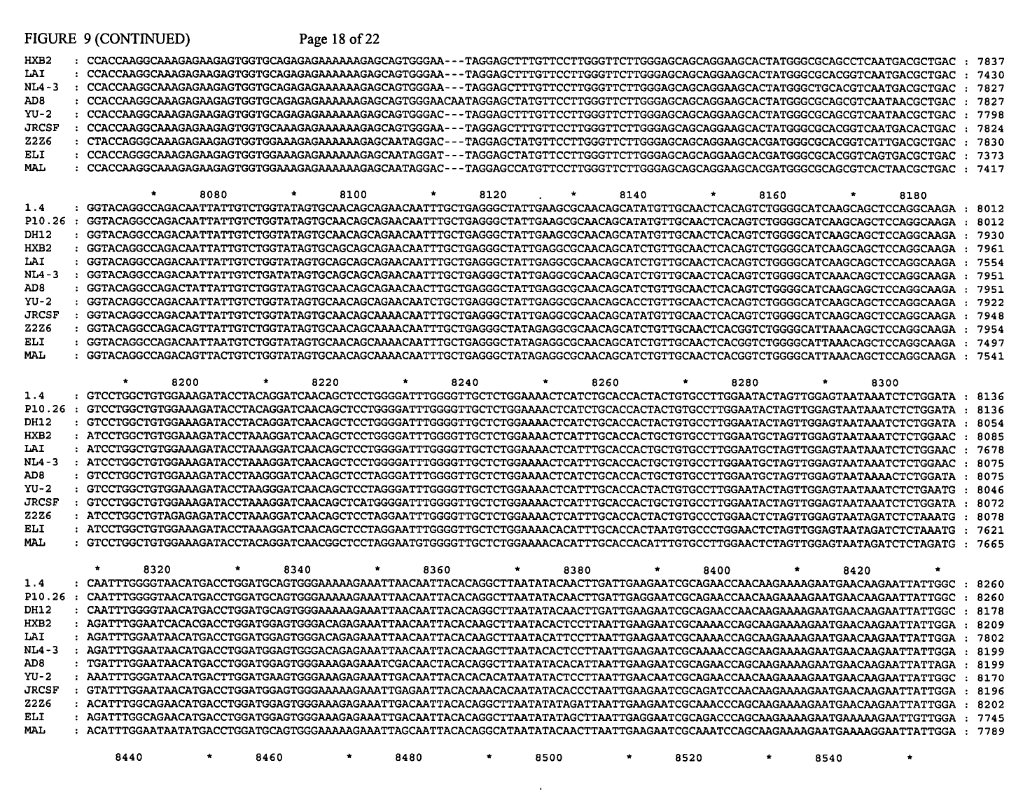

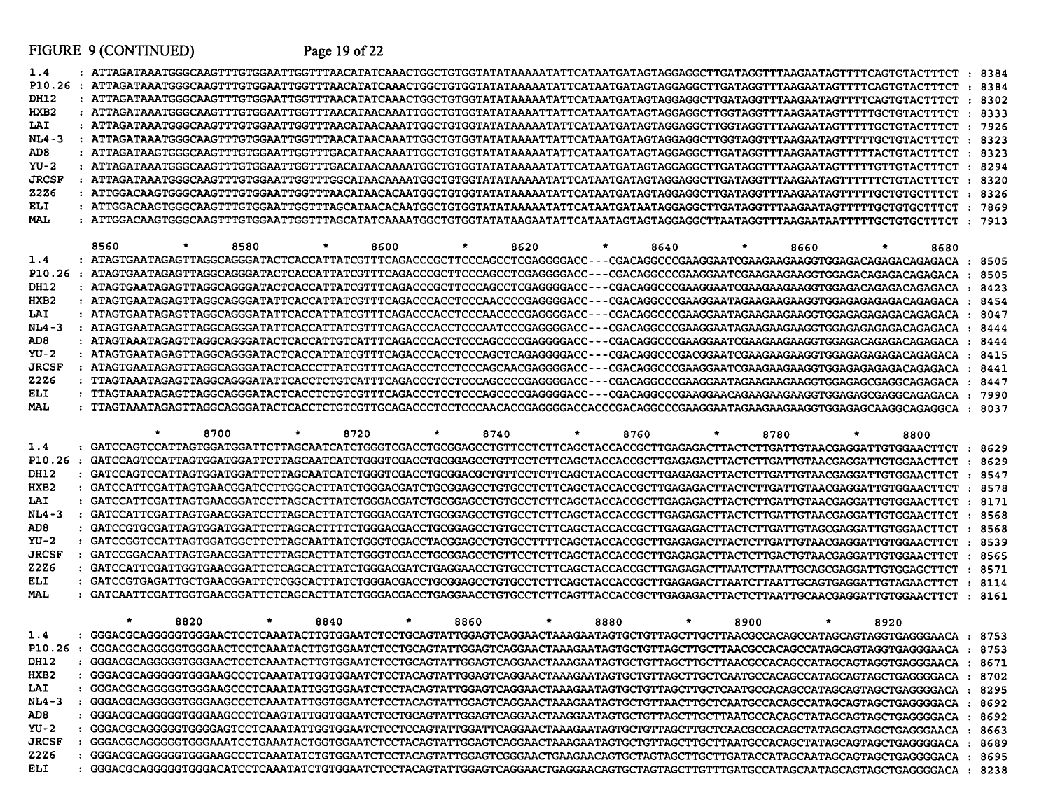

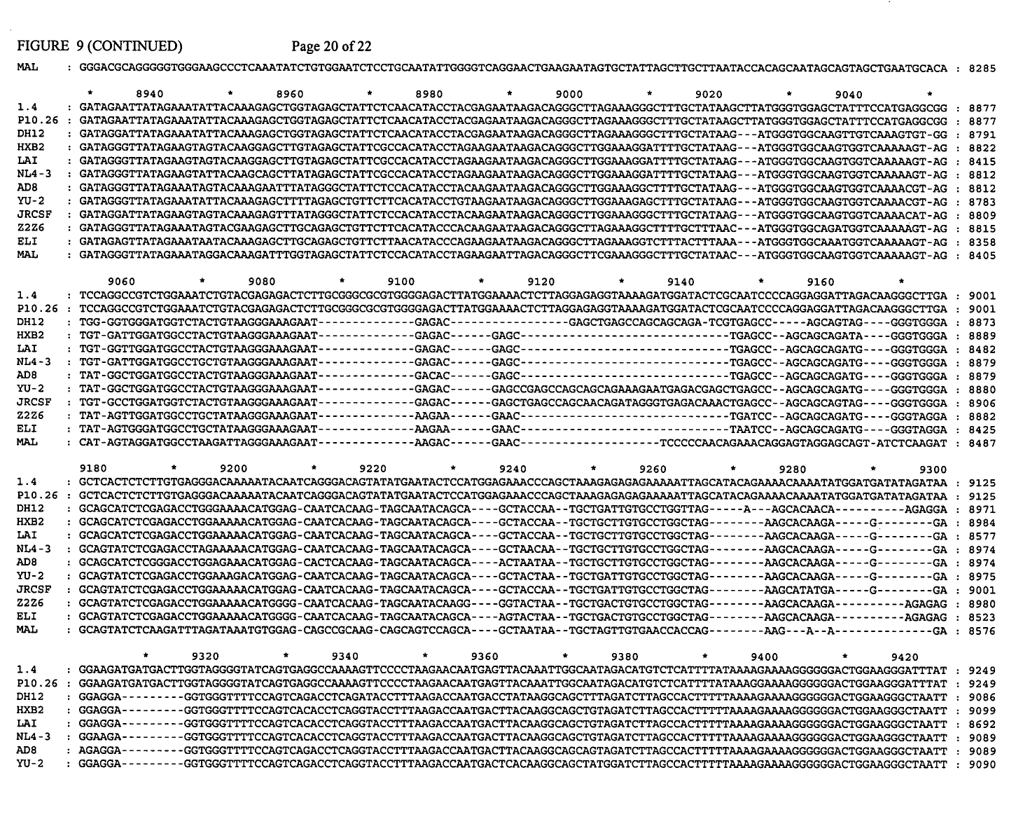

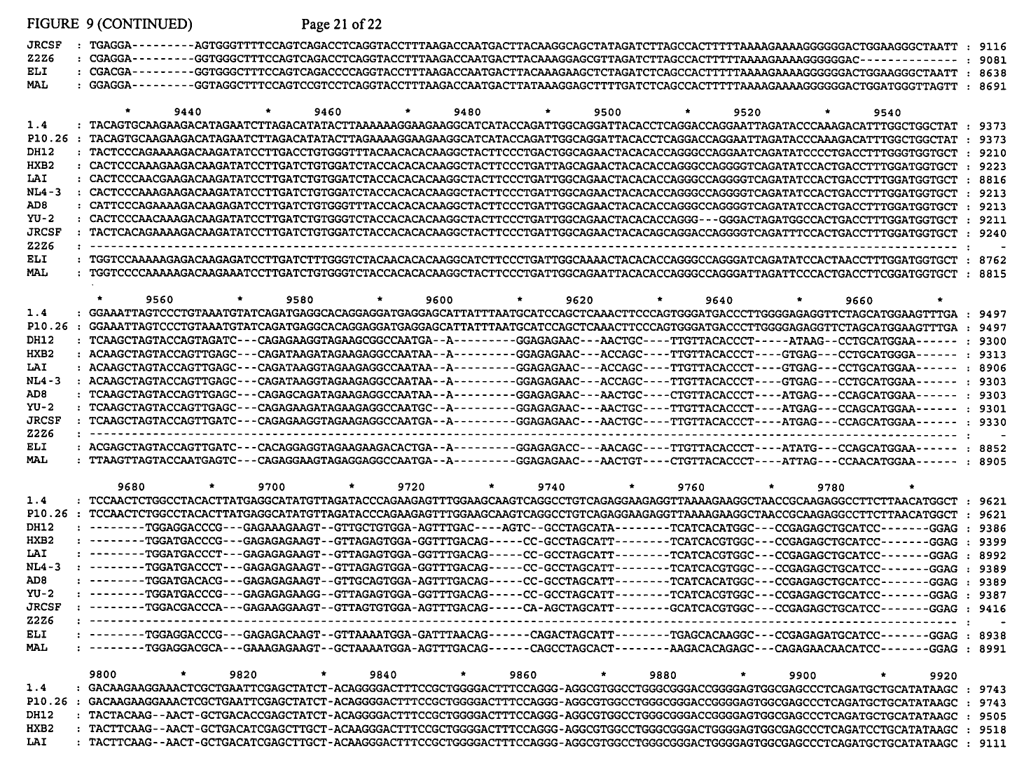

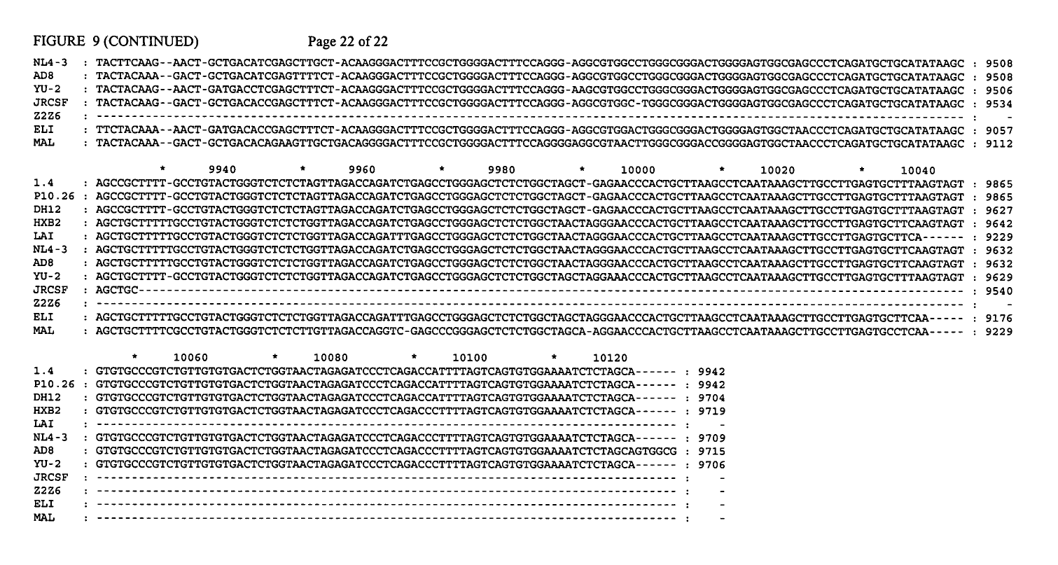

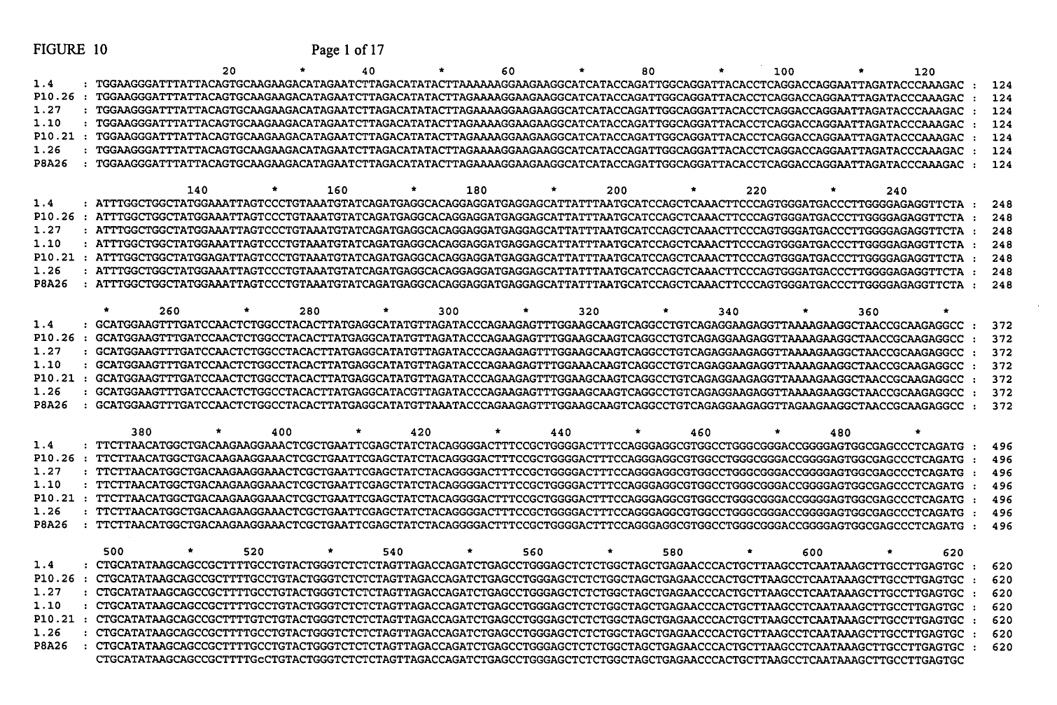

An increase in non-species specific replicative fitness conferred by the sequence changes to 1B3 and the chimeric viruses may account for the observed improvements in pt mPBMC. To investigate this possibility, we compared the replication of these viruses in huPBMC with MD17, using RT normalized viral supernatants to initiate the infections. All the viruses replicated robustly in huPBMC (FIG. 8). Importantly, 1B3 clone 1.4 did not exhibit any increase in viral production over MD17. Although some of the chimeras showed some small improvements (<2 fold), these were not significant as there was no correlation of their replication activities in huPBMC and pt mPBMC. Thus, we conclude that the improvements in pt mPBMC replication were not due to an overall increase in replicative fitness but to specific adaptation to pt mPBMC.

Discussion

HIV-1 variants that can replicate efficiently in macaques will fill an important niche for in vivo models for AIDS (Levy 1996, J. Med. Primatol., 25: 163-174; Nathanson 1999, AIDS, 13 (Suppl A): S113-S120; Joag 2000, Microbes and Infection, 2: 223-229; Nath 2000, Trends Microbiol., 8: 426-431). Such models would enable the testing of drug candidates without many of the concerns arising from the genetic differences between SIV and HIV-1. Furthermore, vaccines based on multiple HIV-1 genes, not just on envelope sequences, can be evaluated with these models. However, the lack of information pertaining to the blocks to HIV-1 replication in macaque cells precludes structure-based, rationally designed solutions. Adaptive approaches rely on the natural ability of virus to generate diversity from which advantageous mutations can be selected and amplified. Although there have been encouraging reports of HIV-1 infection in pig-tailed macaques (Agy 1992, Science, 257: 103-106; Frumkin 1993, Virology, 195: 422-431; Gartner 1994, AIDS Res. Hum. Retroviruses, 10: S129-133; Gartner 1994, J. Med. Primatol., 23: 155-163), the level of virus replication is generally too low to favor successful adaptation to a robustly replicating strain within a reasonable time frame.

With these challenges in mind, we reasoned that supplying sufficient diversity initially would increase the probability that some variants would possess enough of a replicative advantage to ‘spark’ the adaptation process. To accomplish this, DNA shuffling was employed. We shuffled the 3.5 kb fragment encompassing the gag, pro and RT coding regions to which sequences that restrict productive HIV-1 infection of macaque cells have been mapped (Shibata 1995, J. Gen. Virol., 76: 2723-2730). Sequences from 11 HIV-1 isolates, gathered from different clades to increase diversity, were used. The pool of shuffled sequences was cloned into aninfectious MD17 backbone, which was derived from the dualtropic, primary isolate, DH12, and contains the pathogenic “YE” mutant SIV nef (Du Z. 1995, Cell, 82: 665-674). Consistent with previous studies (Agy 1992, Science, 257: 103-106; Frumkin 1993, Virology, 195: 422-431; Gartner 1994, J. Med. Primatol., 23: 155-163; Otten 1994, AIDS, 8: 297-306; Kimball 1998, J. Med. Primatol., 27: 99-103), MD17 and other parental HIV-1 strains replicated marginally and did not persist beyond 2 passages in pt mPBMC (FIG. 1). We subjected 4 gag-pro-RT shuffled libraries to 3 cycles of alternating passages in pt mPBMC and huPBMC, to allow for selection, amplification and adaptation of rare viral species that have acquired replicative advantage. The surviving populations were then stringently selected by performing 4 successive pt mPBMC passages. Of the 4 libraries, only the 1B3 library yielded a variant that survived the selection. This variant was clearly improved over all the HIV-1 parents including MD17, although it still replicated less efficiently than SHIV (SIV genome with HIV-1 env). The 1B3 variant could replicate to high levels (>100 ng/ml p24) and most importantly, it could be passaged continuously in fresh pt mPBMC.

Several full-length proviral clones of 1B3 that exhibited the improved replication phenotype were obtained. They all possessed a similar structure in the shuffled region, which was predicted to result from recombination between sequence fragments of at least 7 of the parents (FIG. 6a). Several changes were found in the unshuffled regions of the genome, many of which were shared among the different clones. These clones likely arose from a single founder, generated by shuffling, which then acquired further adaptive mutations. Functional analyses of reciprocal chimeras between a representative improved 1B3 clone and the parental MD17 demonstrated that both the shuffled and unshuffled regions of 1B3 contributed synergistically to the augmented replication phenotype in pt mPBMC. The shuffled gag-pro-RT and int→env regions of 1B3, when transplanted individually into the original MD17 background, resulted in observable improvements in pt mPBMC replication, although replication levels were significantly lower than that for the complete 1B3 clone. Similarly, when these sequences in 1B3 were replaced by MD17 sequences, replication was adversely affected. The evolved nef-LTR of 1B3 had a smaller effect. The enhanced replication of 1B3 and the chimeric viruses were not due to an overall increase in replicative fitness since they exhibited no significant improvement in huPBMC compared to MD17.

The observation that many changes in different regions were required may reflect multiple limiting steps to HIV-1 replication in pig-tailed macaque cells. Additionally, it may reflect the requirement for different HIV-1 functions that interact with one another to be coordinately altered. This highlights the complexity involved to successfully evolve HIV-1 for pig-tailed macaque cell tropism. That only one shuffled species from the 4 libraries, containing an estimated 2.4×10⁵ total number of clones, successfully survived the selection regime and adapted, suggests that improved solutions are rare and complex. Thus, natural mechanisms of retroviral recombination and mutation are unlikely to generate these improved variants easily. The control mixture of HIV-1 parents, when subjected to the same selection became extinct (FIG. 3). Similarly, we have never achieved any observable improvements by extensive passaging of the best parent, MD17, alternately in pt mPBMC and huPBMC (data not shown). By screening larger libraries of shuffled HIV-1, other improved recombinants may be recovered.

It cannot be ruled out that unshuffled gag-pro-RT sequences from a HIV-1 parent, when transplanted into the 1B3 backbone, may also result in the fully improved replication phenotype of the complete 1B3 virus. However, in the absence of a priori information on which gag-pro-RT sequences are favorable, DNA shuffling provides an efficient means to rapidly generate and screen many permutations for the best solution. Furthermore, the fragmentation of parental sequences during the shuffling process rarely proceeds to completion. Thus, it is likely that residual, full-length parental sequences were constituents of the screened libraries. That a recombinant gag-pro-RT sequence was ultimately selected over all the other constituents in the libraries, suggests that it conferred the best advantage.

In contrast to other SHIV constructs that are composed of greater than 50% SIV sequences, all the sequences in 1B3 except for SIV nef were HIV-1 derived. Although 1B3 accumulated numerous point-mutations, these changes did not shift its composition towards a more SIV-like sequence. Gartner et al. (Gartner 1994, J. Med. Primatol., 23: 155-163) reported that a HIV-1 strain, CH69, derived from a chimpanzee persistently infected with HIV-1 strain IIIB, showed improved viral production in pigtailed macaque cells. This variant acquired changes during its adaptation in the chimpanzee host in vivo that likely conferred replicative advantages in pigtailed macaques as well. CH69 thus underwent a different evolutionary route compared to 1B3. The activity of the evolved 1B3 virus is currently being assessed in vivo. This improved variant should be useful in establishing a macaque model of HIV-1 infection.

### EXAMPLE 2

### Isolation of Additional Clones

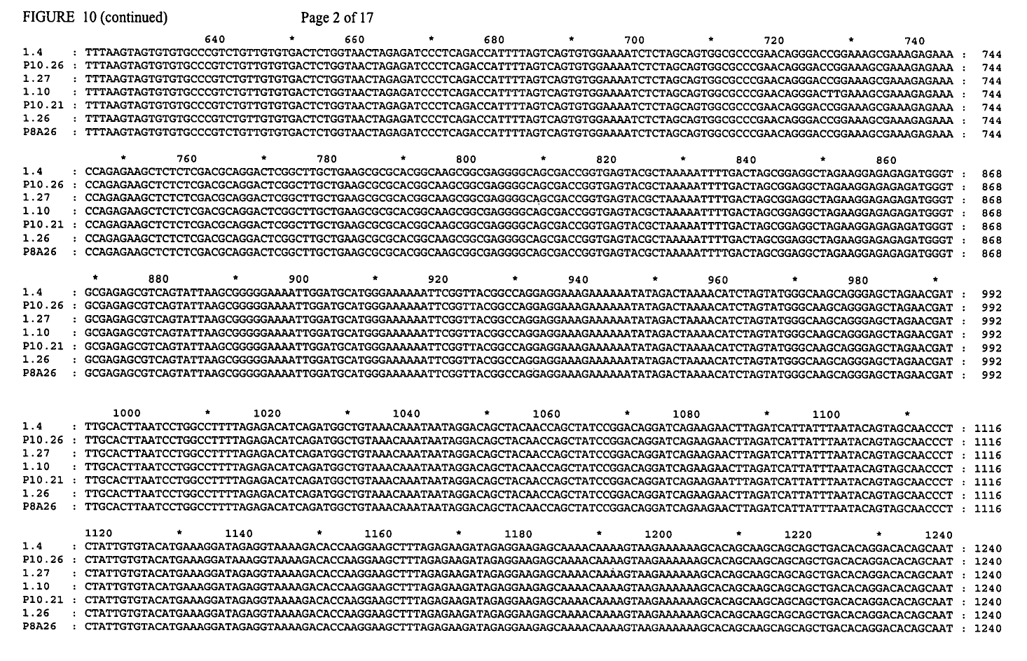

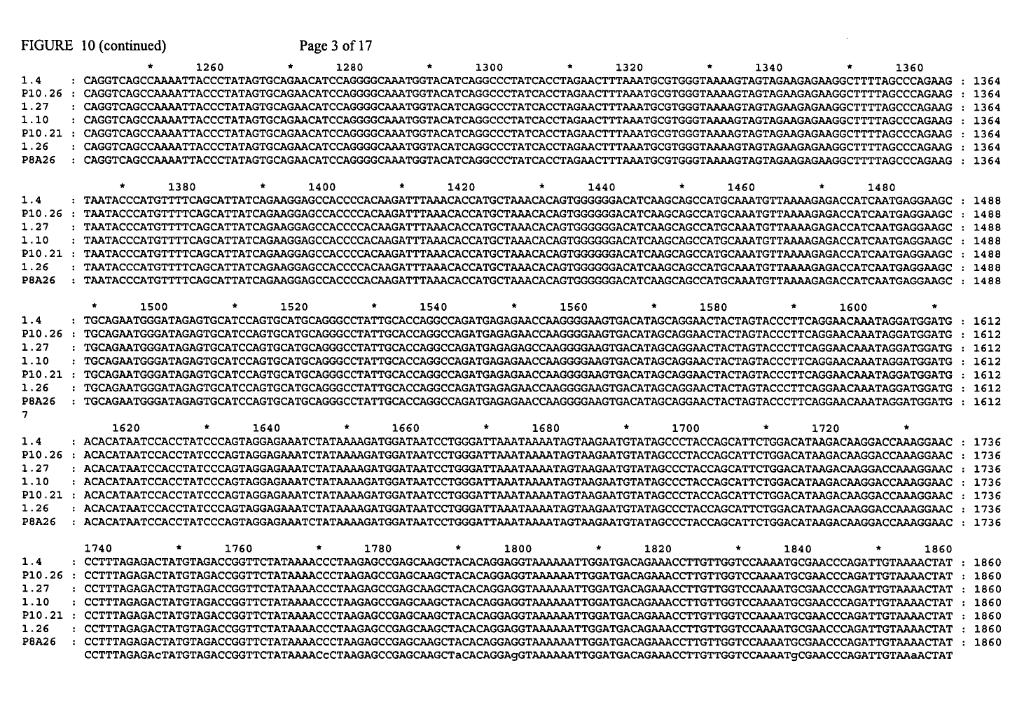

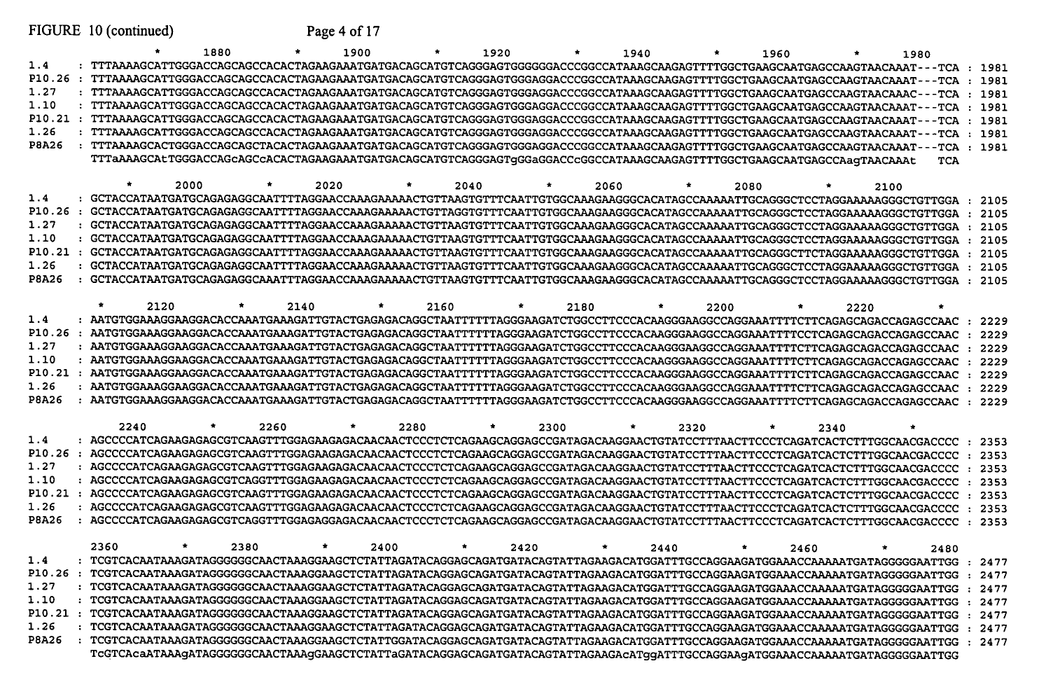

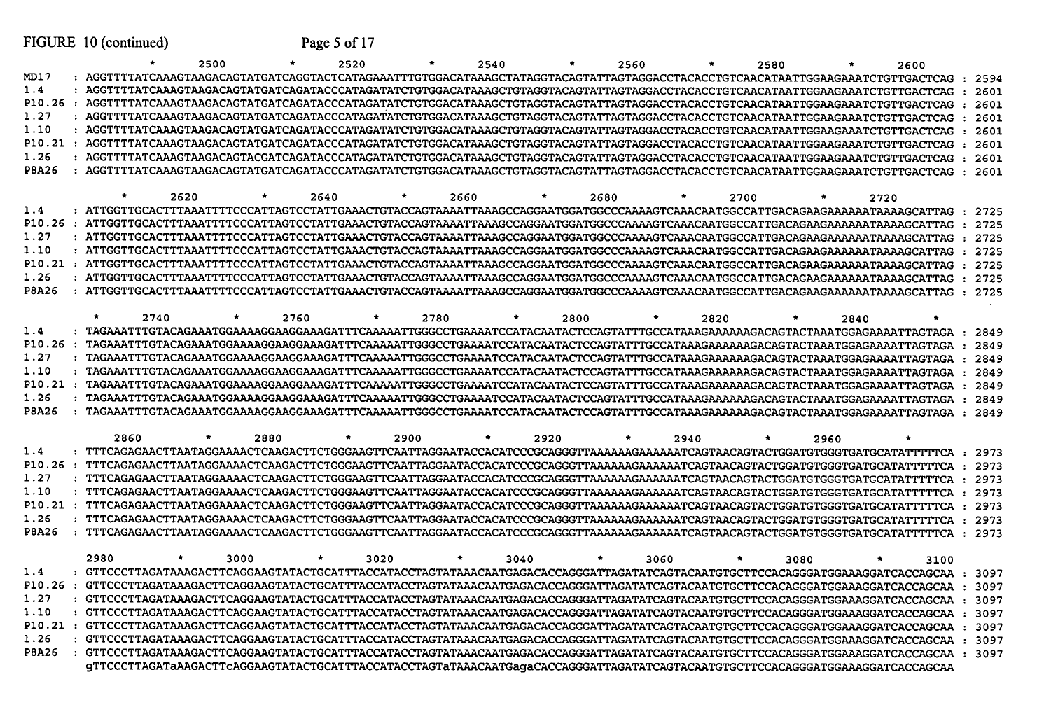

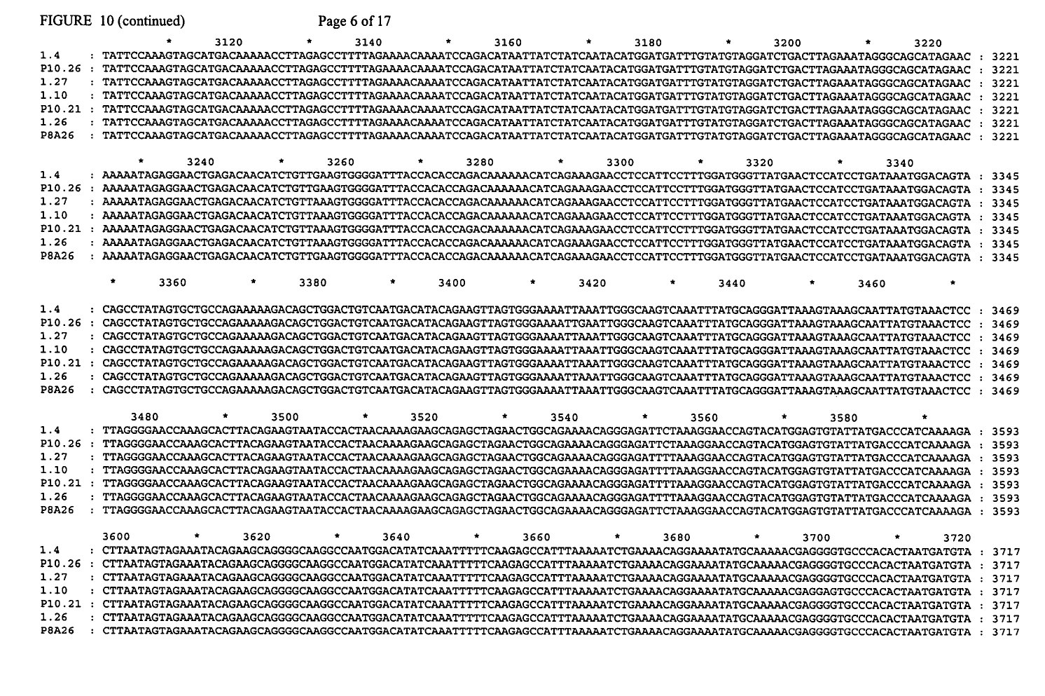

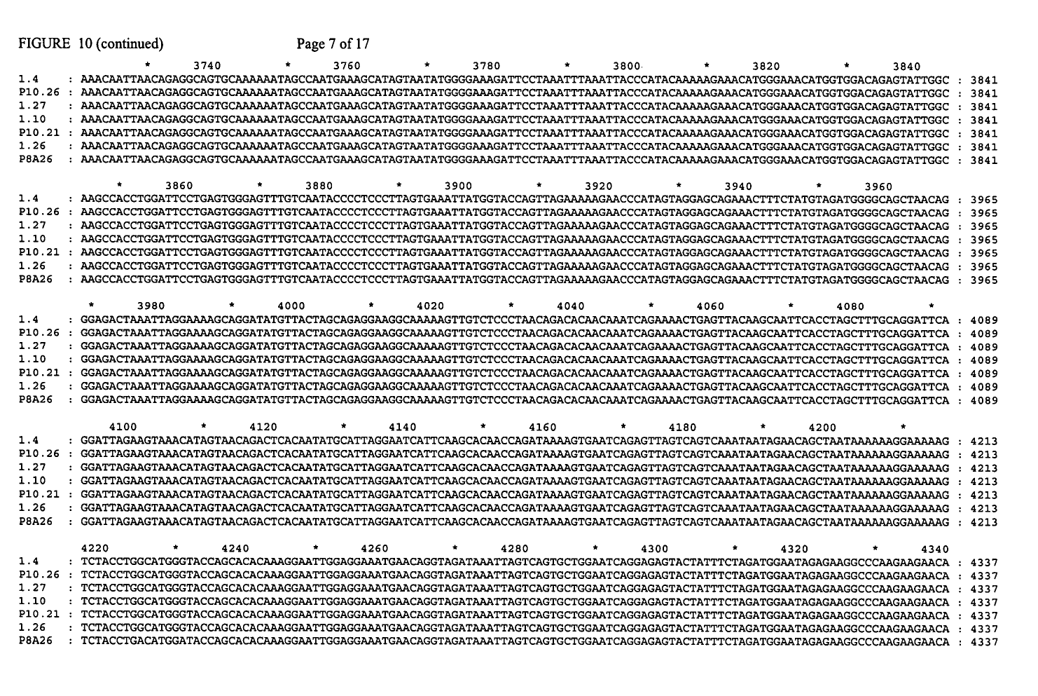

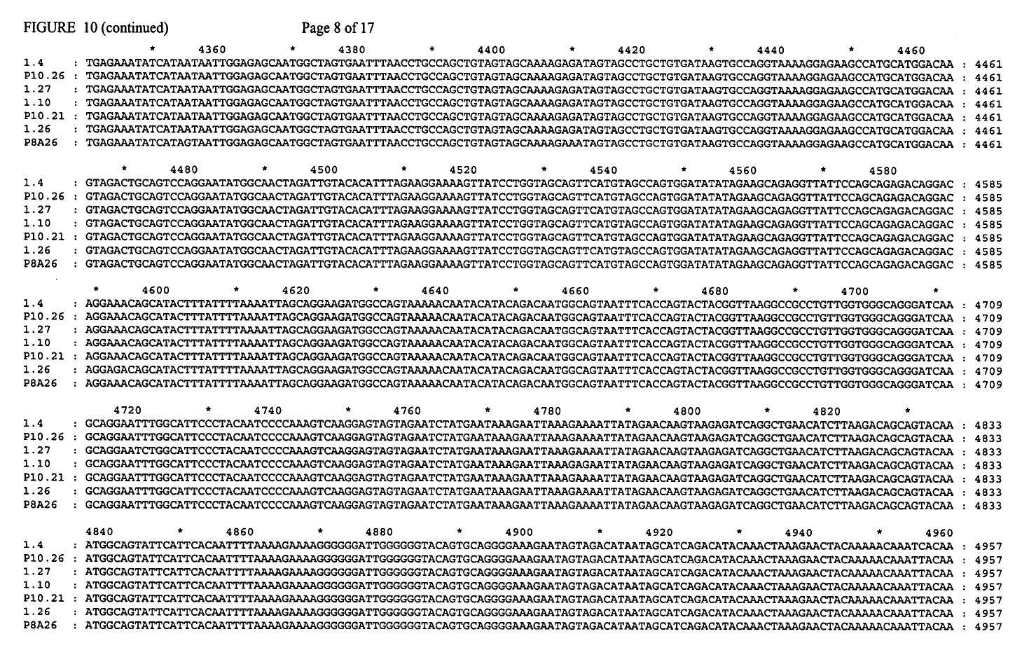

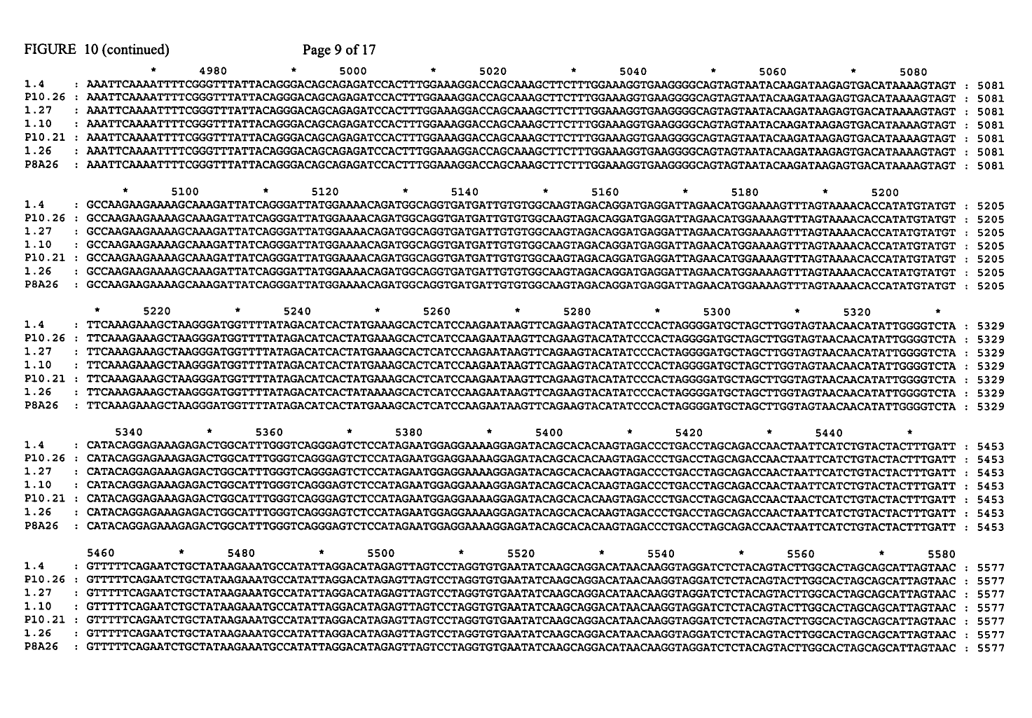

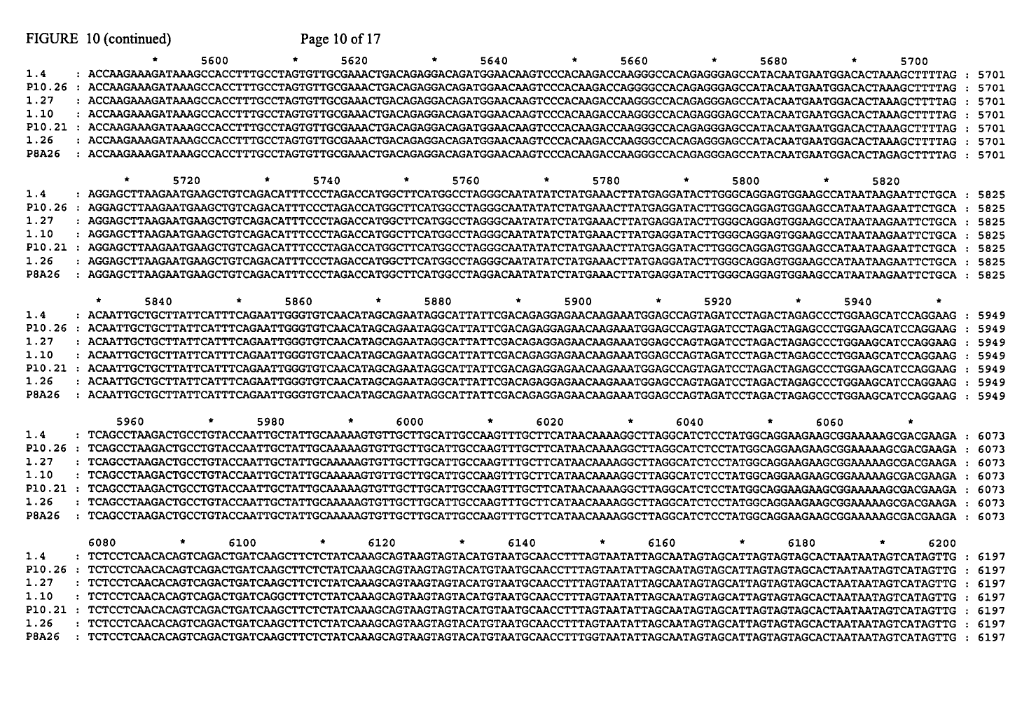

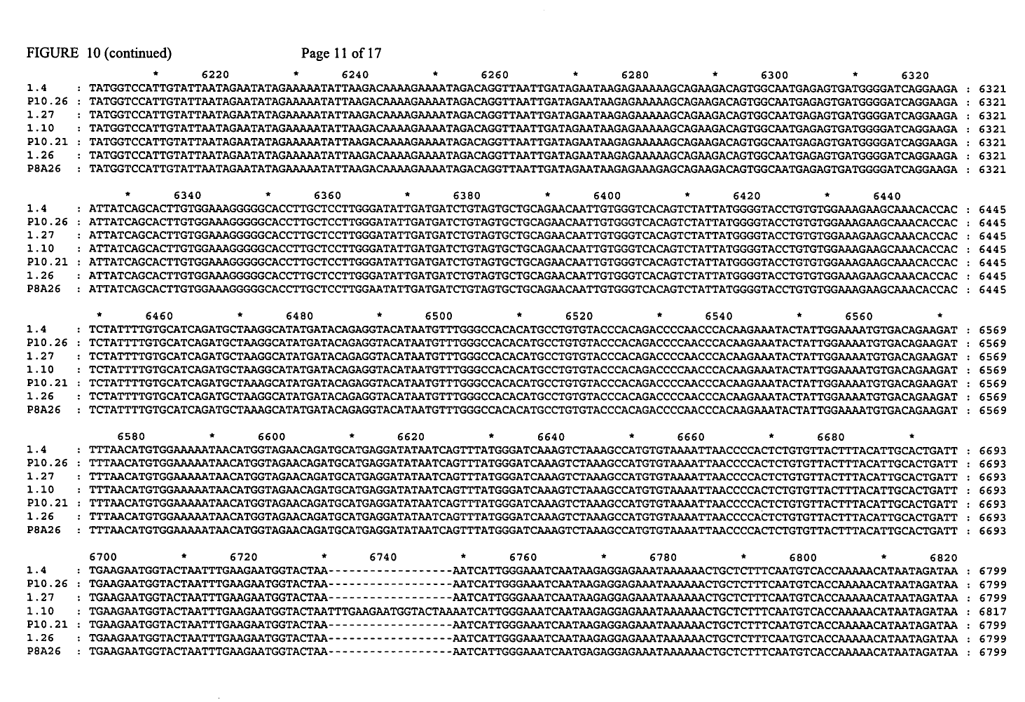

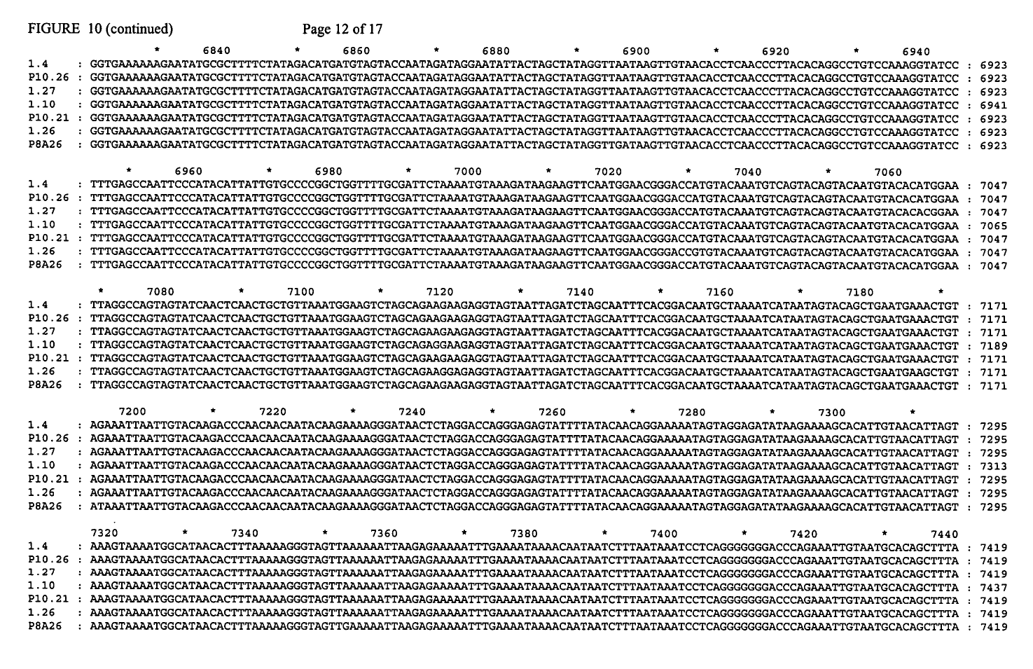

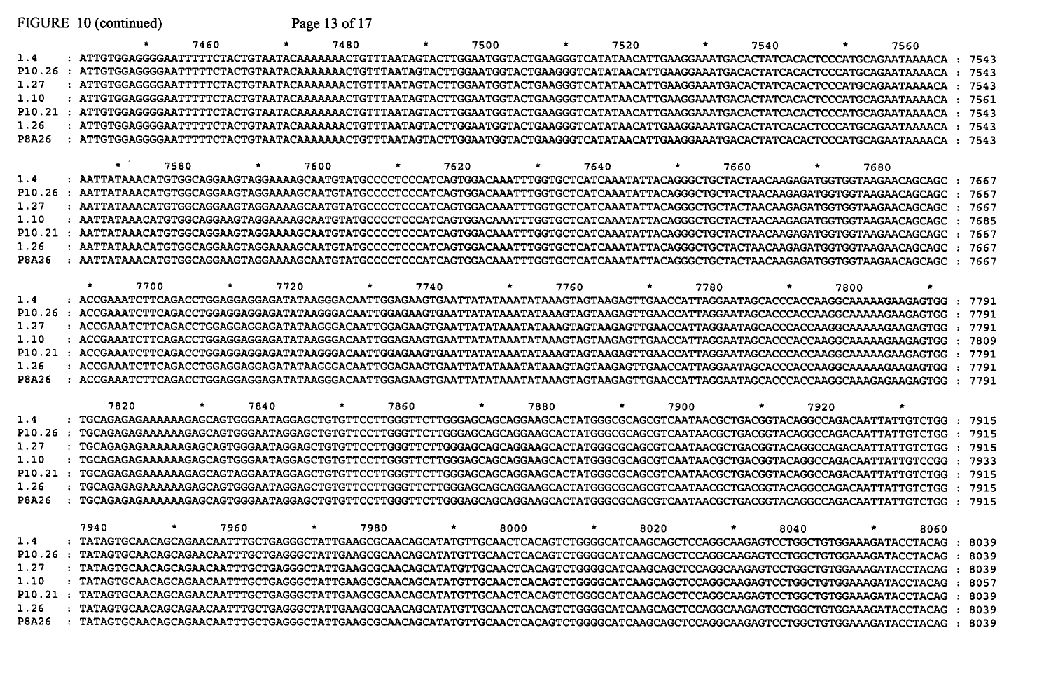

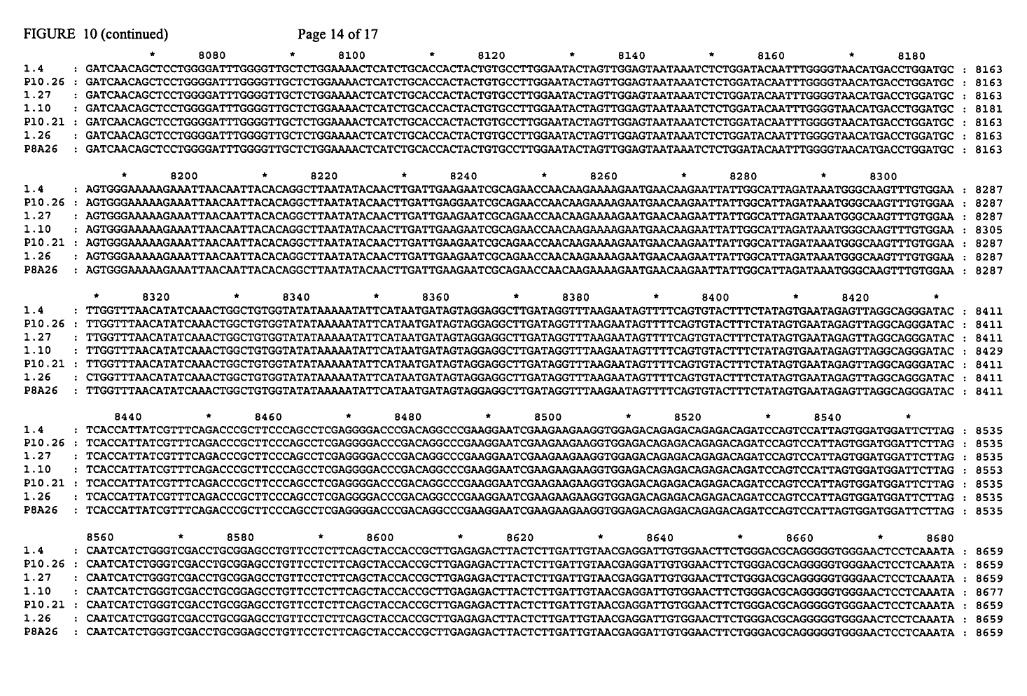

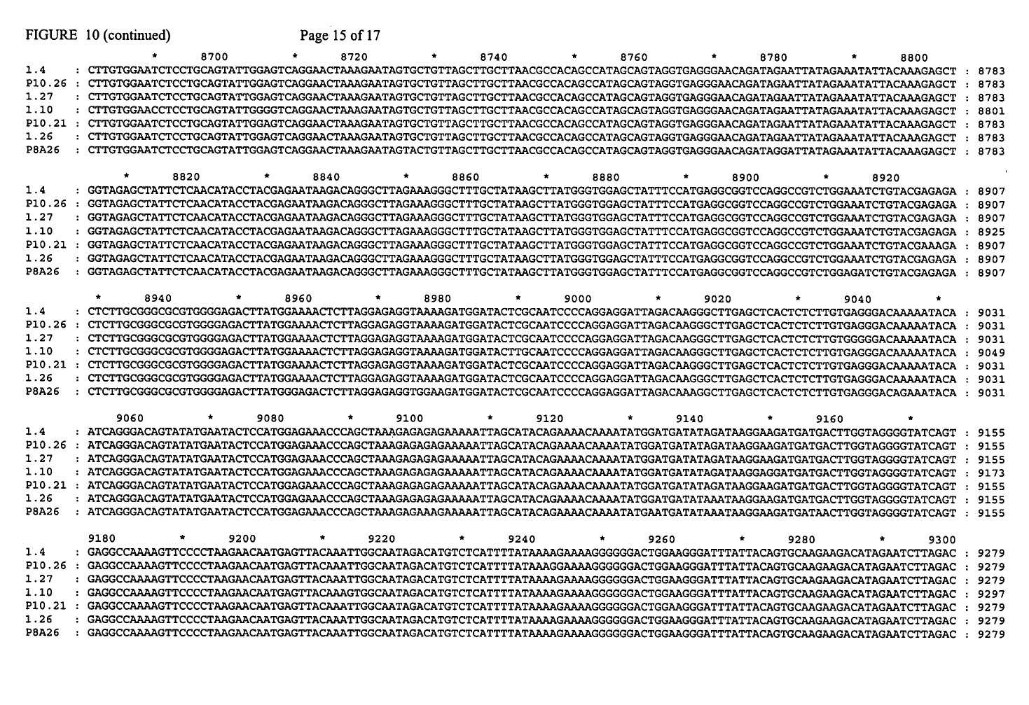

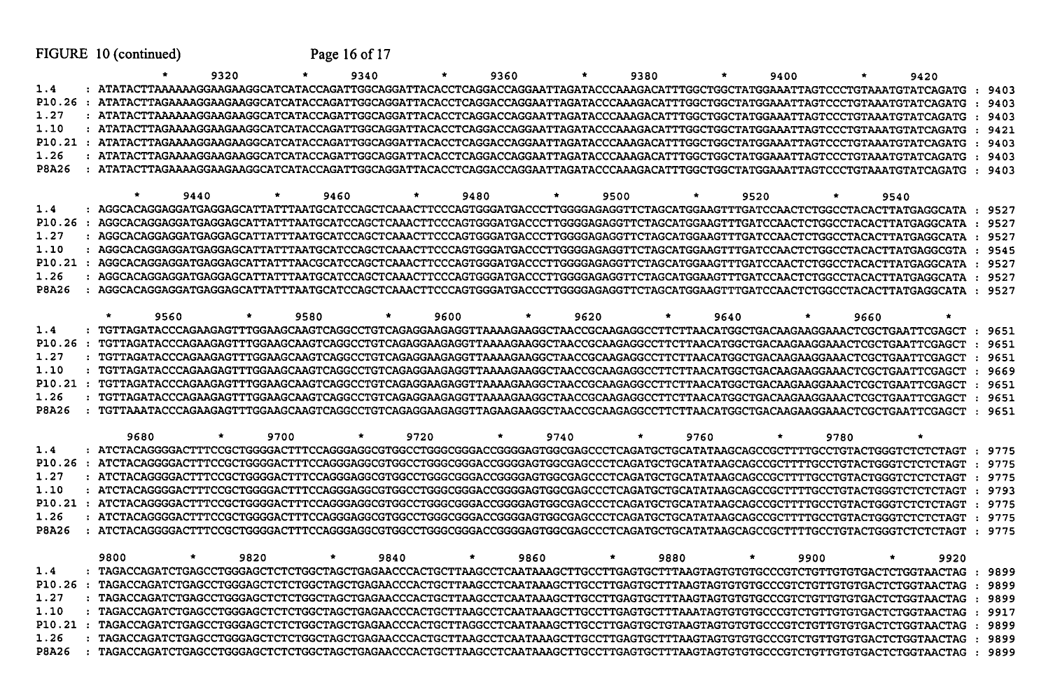

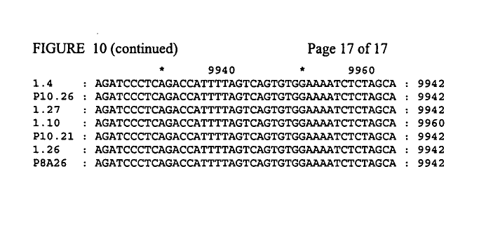

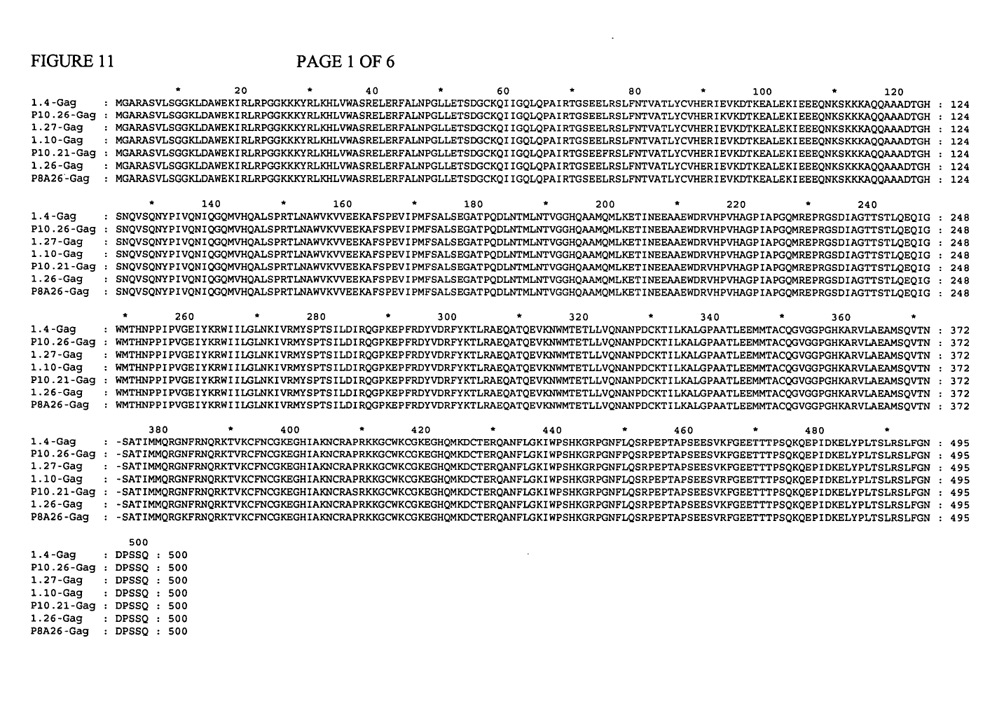

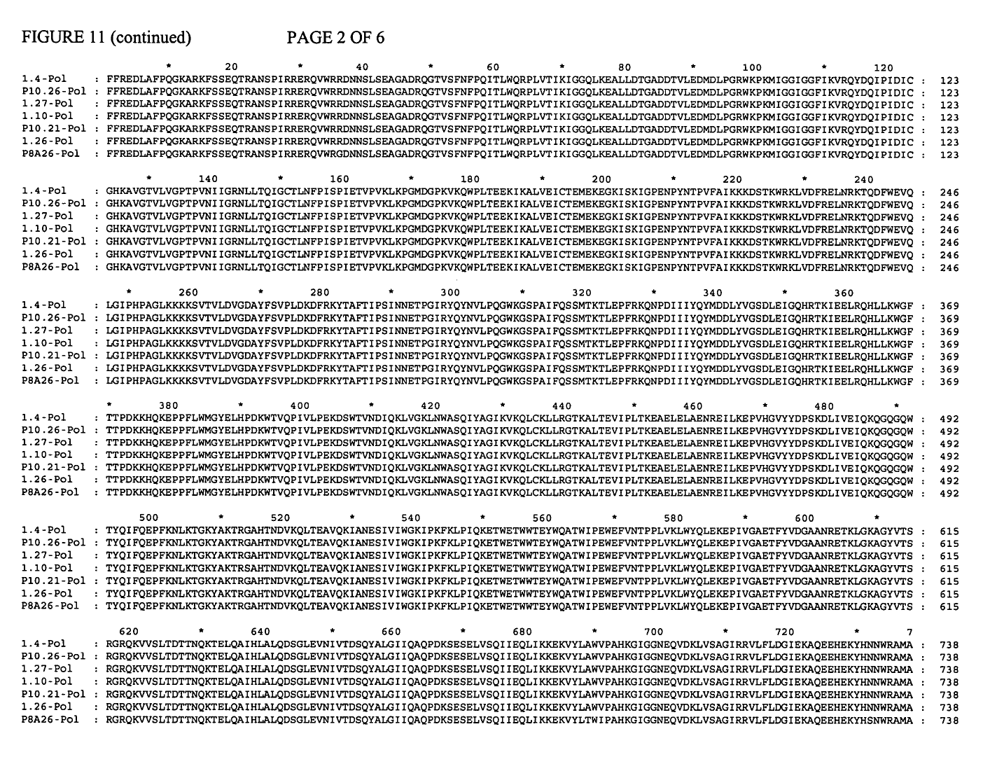

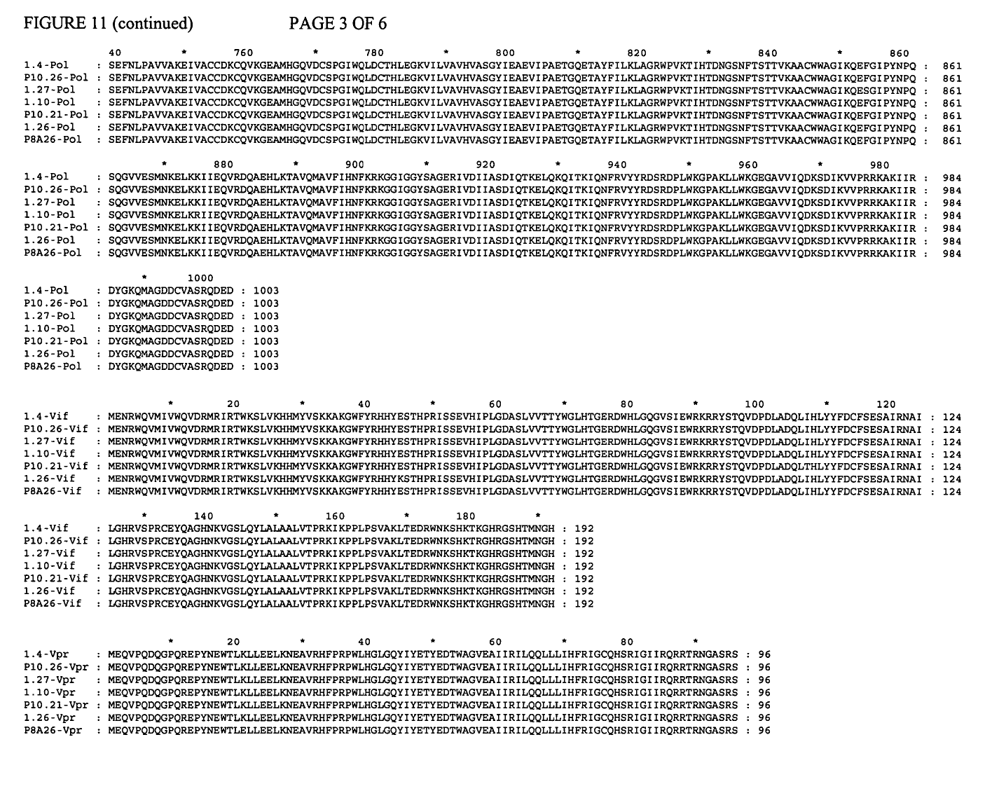

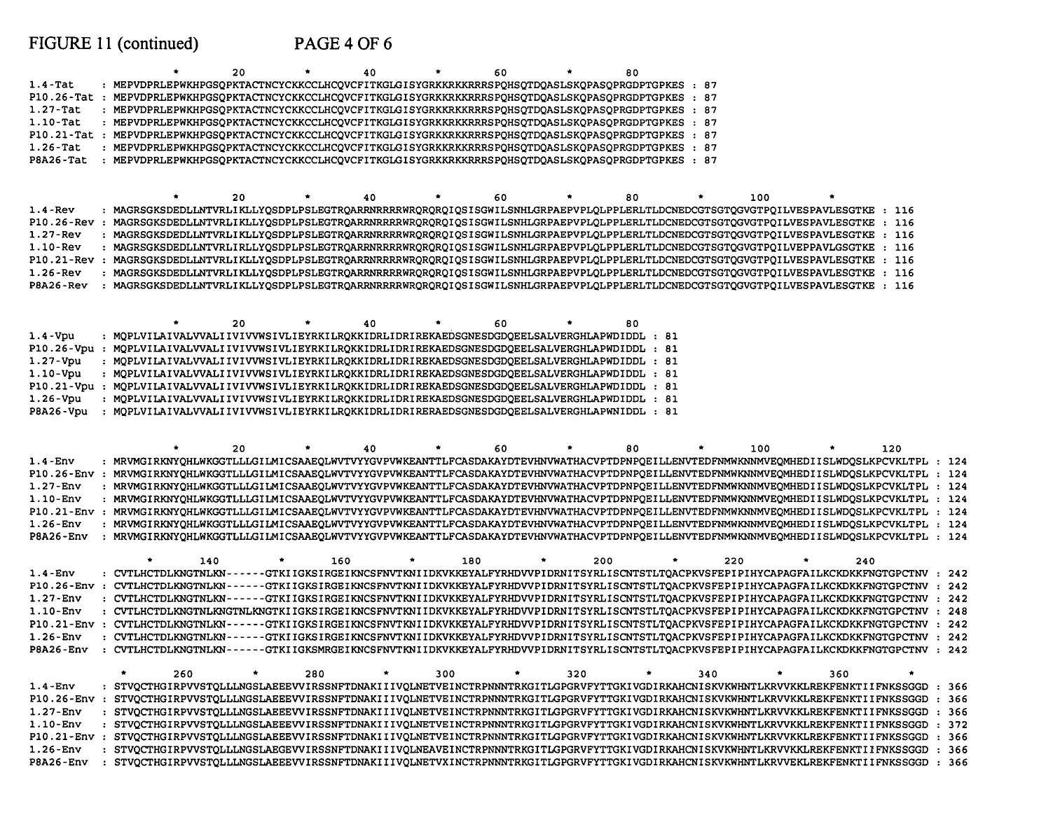

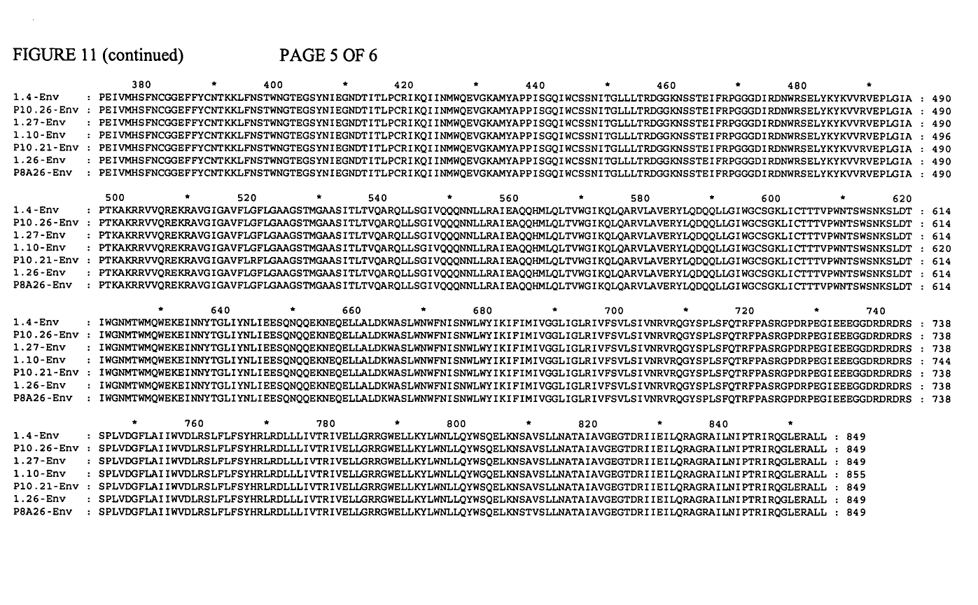

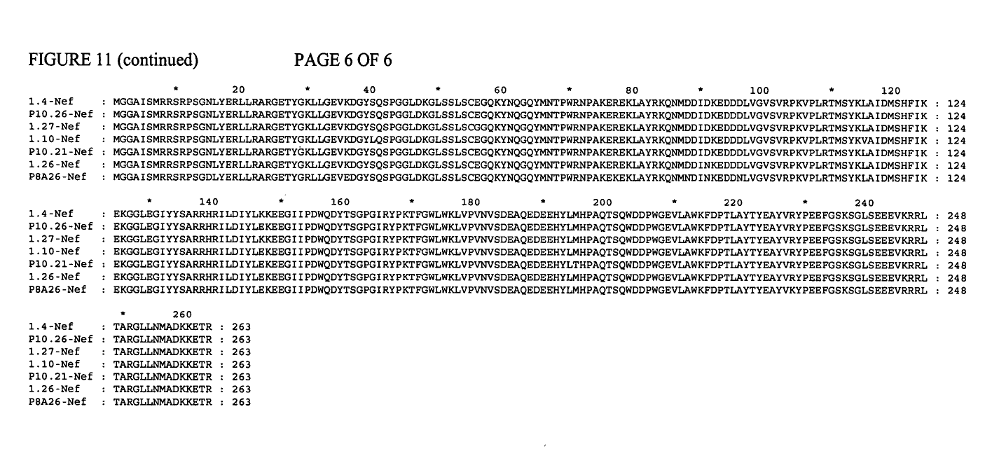

Several full-length clones of the 1B3 pool were isolated following the strategy for isolating clone 1.4. The polynucleotides sequences of the seven isolated clones (1.4, P10.26, 1.27, 1.10, P10.21, 1.26, and P8A26) are aligned in FIG. 10. The amino acid sequences of the nine HIV-1 polypeptides of each clone are aligned in FIG. 11.

### EXAMPLE 3

### Preparation of the Modified or Recombinant Virus Inoculum for Animal Challenge

In order to inoculate a pig-tailed macaque (Macaca nemestrina) with a high-titered recombinant or modified virus of the invention (e.g., 1B3 virus; a modified virus comprising the nucleic acid of claim 1, 2, 5, 7, or 9, or another chimeric virus variant of the invention), the virus is first propagated in vitro, in cultured monkey peripheral blood mononuclear cells (PBMC) prepared from the monkey that is to be challenged with the virus (or propagated virus). A procedure for making the cultured monkey PBMCs is set forth below.

At the peak of virus replication, about 5 milliliters (ml) of the inoculum is injected intravenously into the blood or into the spleen or bone marrow of a pig-tailed macaque monkey. The inoculum comprises virus-infected cells and/or viral supernatant (e.g., supernatant into which the cells have secreted the virus). If desired, a larger or smaller volume of inoculum can be used for inoculation. The volume of inoculum (virus-infected cells and/or viral supernatant) to be used depends on the pathogenicity of the chimeric virus. The greater the pathogenicity of the virus, the less number of virus particles (or cells infected with the virus) that need to be delivered to the animal to cause infection.

In this example, the cells and virus-containing supernatant are injected intravenously into the blood of the monkey. The inoculum typically contains, e.g., from about 1 million to about 5 million infected cells and/or 10,000 to about 500,000 infectious virus particles per milliliter. The timing of in vitro infection and in vitro challenge is determined by a preliminary experiment, so that the maximum amount of virus is delivered to the animal. To obtain higher titers of virus in supernatant, the virus can also be cultured using phytohemmaglutinin (PHA)-stimulated human PBMCs. However, in this instance, only the resulting high-titer supernatant and not infected human PBMC are used for inoculating the macaque monkey.

Procedure for making cultured PBMCs. Cryopreserved PBMCs are thawed and stimulated with phytohemmaglutinin (PHA). (Alternatively, freshly purified PBMCs stimulated with PHA can be used.) Three days later, 10 million stimulated PBMC are inoculated with the modified or recombinant virus of the present invention (e.g., a 1B3 virus, such as 1.4 viral clone, or another chimeric virus variant of the invention). To determine time required for maximum virus production, the amount of virus released into the culture medium is monitored daily using a HIV p24 antigen kit (Beckman-Coulter) according to the manufacturer's instructions. Typically, the peak viral production occurs between 7 and 14 days after infection (referred to as Time “Day X” in Table 1 below), and such a culture can contain 1-5 million of infected cells and 10,000 to 500,000 infectious virus particles per milliliter (ml).

TABLE 1
Preparation of challenge virus and the timing of challenge

| Time | Action |
|---|---|
| Day X | Infect stimulated PBMC with modified or |
| | recombinant virus (e.g., a 1B3 virus, such |
| | as 1.4 viral clone, or another chimeric virus |
| | variant of the invention) |
| Day 0 | Inoculate the monkey with the infected |
| | PBMC culture and/or viral supernatant |

### EXAMPLE 4

### Animal to Animal Passage

The present invention also provides methods of further improving the in vitro replication properties of the chimeric viruses of the invention in pig-tailed macaque monkeys. For example, if desired, the modified or recombinant virus (e.g., 1B3 virus, such as 1.4 viral clone, or other chimeric virus variant of the invention described herein) can be made further pathogenic by additional adaptation of the virus in vitro in such monkeys. Replication of immunodeficient viruses in vitro is most efficient during the acute phase of infection that typically lasts for a few days to a few weeks, until the specific immune response is developed. Experimental animal models and clinical studies have shown that high virus loads during this period result in sustained high virus loads during the chronic infection period and earlier development of immune deficiency. Two factors determine robustness of acute infection in vitro: replication capability of the virus itself and the ability of the host to mount specific immune responses.

The shuffled chimeric viruses described herein can be made to replicate more robustly in vitro in a macaque monkey by suppressing the monkey's (host) immune system during the acute infection period using an anti-CD8 monoclonal antibody that suppresses cytotoxic T cell response. The immune suppression allows the virus to replicate to high titers, such that the frequency of adaptive mutation and recombination increases; resulting virus population is then analyzed for one or more altered (mutant) viruses that cause immunodeficiency in the host monkey without anti-CD8 monoclonal antibody treatment. This is tested by performing animal to animal passage of the recombinant, chimeric virus with and/or without immunosuppressive treatment (described below).

In the first experiment, one pig-tailed macaque monkey is inoculated with the original 1B3 virus (or other chimeric virus variant of the invention) under anti-CD8 monoclonal antibodies (mAb) treatment. If this first monkey shows sustained loss of CD4 positive cells (<200 CD4 positive cells/microliter (ul) blood for 3 consecutive time points) associated with high virus loads (1,000,000 RNA copies/ml plasma), the virus isolated from the latest blood sample and 5 ml of freshly collected whole blood are used to inoculate four naïve monkeys (passage 2). Two of the 4 monkeys are treated with anti-CD8 mAb as described above. If a monkey without anti-CD8 mAb treatment shows a loss of CD4 positive cells in the passage 2, the virus isolated from that monkey is used to inoculate three naive monkeys without anti-CD8 mAb antibodies (passage 3). If 3 out of the 3 monkeys show a loss in CD4+ T cell count, a pathogenic virus has likely been generated, and further genetic and functional analysis of the virus is performed. If only anti-CD8 mAb-treated monkeys show a CD4+ T cell count loss in the passage 2, the virus isolated from the latest blood sample and 5 ml of freshly collected whole blood are used to inoculate four naive monkeys; two of the 4 monkeys are treated with anti-CD8 mAb as described above (passage 2+).

Passaging is continued until a CD4+ T cell count loss is observed in 3 out of 3 monkeys without anti-CD8 mAb treatment. If the first monkey does not show sustained a loss in CD4+ T cell counts until month 6, another naive monkey is injected with a mixture of the original 1B3 virus and viruses isolated from the first monkey at various time points, under anti-CD8 mAb treatment as described above. If neither monkey shows a loss in CD4+ T cells in the subsequent 6 months, the third monkey is used. The procedure is continued until 6 months after the fourth monkey is inoculated.

An alternative method for increasing the diversity of virus inoculum and/or to boost viral titers involves recovering viruses from infected pig-tailed macaque monkeys, culturing the recovered viruses in vitro to boost titers, and then re-inoculating this high titer virus into the next pig-tailed macaque monkey. Combinations of direct monkey to monkey passages and intervening culturing of virus can be used. Passaging of virus between monkeys may also be expanded to include other tissues, e.g., bone marrow, spleen, and purified PBMC in addition to whole blood.

### EXAMPLE 5

### Immunosuppressive Treatment of Monkeys

In one method, a variant of a chimeric virus of the invention (e.g., 1B3 virus, a modified virus comprising the nucleic acid of claim 1, 2, 5, 7, or 9, or other chimeric virus variant of the invention described herein) that is more pathogenic in pig-tailed macaque monkeys (e.g., replicates more robustly) is made by treating, e.g., a 1B3 virus-infected monkey (e.g., infected with a modified virus comprising the nucleic acid of claim 1, 2, 5, 7, or 9) with an immunosuppressive agent, anti-CD8 monoclonal antibody (anti-CD8 mAb), as described by Igarashi et al., Proc. Nat'l Acad. Sci. 96:14049-14054 (1999), which is incorporated herein by reference in its entirety for all purposes. The presence of anti-CD8 mAb suppresses an anti-HIV immune response mediated by CD8 positive cytotoxic T-lymphocytes and assists in sustaining high levels of virus replication. Table 2 shows the schedule of anti-CD8 mAb administration. Viral load and disease development are monitored in the infected animal (e.g., such as during the acute infection phase). Anti-CD8 mAb administration can be withdrawn after a determined time period (see, e.g., Igarashi et al., Proc. Nat'l Acad. Sci. 96:14049-14054 (1999), in which the animal developed immunodeficiency 7 months after anti-CD8 mAb treatment was withdrawn).

Following treatment, the virus is isolated from the animal using standard procedures and sequenced. The isolated virus is a variant of the original chimeric virus if it comprises a sequence that is a variant of the original chimeric virus. The variant virus is then tested for increased pathogenicity by inoculating one or more additional monkeys that have not undergone anti-CD8 mAb treatment with the virus variant. The pathogenicity of the virus variant has increased if monkeys to which the variant virus is administered develop disease symptoms more readily or to a greater extent than does a monkey to which the original chimeric variant is administered without anti-CD8 mAb-treatment.

TABLE 2
Immunosuppressive treatment of monkeys

| Time | Action |
|---|---|
| Day −1 | Intravenous administration of anti-CD8 |
| | mAb (2.5 mg) |
| Day 0 | Virus challenge, anti-CD8 mAb |
| | administration (0.5 mg) |
| Day 2 | Anti-CD8 mAb administration (0.5 mg) |
| Day 4 | Anti-CD8 mAb administration (0.5 mg) |
| Day 6 | Anti-CD8 mAb administration (0.5 mg) |

### EXAMPLE 6

### Macaque Monkey Model and Measurement of Virological, Immunological and Clinical Parameters

The modified or recombinant sequences of the invention are adapted to replicate in macaque monkey (Macaca nemestrina) primary lymphocytes in vitro as described above. In some embodiments, the modified or recombinant sequences replicate in a population of macaque monkey cells (e.g., pigtailed macaque monkey cells) in vitro at a greater rate or for a longer period of time than that of a parental or known HIV-1 virus. Each of these chimeric viruses is used to generate a macaque monkey model of HIV-1 infection and/or AIDS-associated disease. Briefly, an amount of a chimeric virus sufficient to induce immunodeficiency and/or disease is administered to a macaque monkey by intravenous injection. The amount of virus administered is that which is, for example, sufficient to cause immunodeficiency after the injection from about 10 TCID₅₀ (or TCID50) to about 5×10⁵ TCID₅₀ (50% tissue culture infectious dose), from about 10 TCID₅₀ to about 10⁵ TCID₅₀, from about 300 TCID₅₀ to about 10⁴ TCID₅₀, from about 300 TCID₅₀ to about 10⁴ TCID₅₀, from about 500 TCID₅₀ to about 10⁴ TCID₅₀, as described by the methods and procedures in Reed, L. J. et al., Am. J. Hyg. 27:493-497 (1938), which is incorporated herein by reference in its entirety. The onset of immunodeficiency and/or disease symptoms in the infected monkey is assessed by measurement over time of various virological, immunological and clinical parameters. Human AIDS patients typically show, e.g., high levels of HIV infection, loss of CD4 positive T cells, and a variety of clinical symptoms, such as wasting, opportunistic infections, and neurological disorders. To examine the pathogenicity of the chimeric virus in the monkey, the infected monkey is closely monitored for plasma recombinant virus load (e.g., 1B3 virus load), CD4 T cell count, anti-HIV antibody response, and clinical symptoms, as described in, e.g., Shibata et al., J. Infectious Disease 176:362-373 (1997), which is incorporated herein by reference in its entirety for all purposes. Using 2-10 ml blood obtained from the infected monkey, a variety of assays are performed, including flow cytometry to measure CD4 positive T cell counts, ELISA to measure the presence of anti-HIV antibodies, reverse transcriptase polymerase chain reaction (RT-PCR) to quantify plasma virus load, DNA PCR to quantify PBMC virus load, and isolation of infectious virus (see Section 5 below), as described in, e.g., Shibata et al., J. Infectious Disease 176:362-373 (1997).

Table 3 shows the schedule of blood collection from the injected monkey. Clinical examination is performed at times of blood collection.

TABLE 3
Blood collection schedule and diagnostic assays

| | Blood | |
|---|---|---|
| Time | Vol. | ASSAY |
| Day −14 | 2 ml | CD4+ T cell count, plasma viral RNA |
| Day −7 | 2 ml | CD4+ T cell count |
| Day 0 | 5 ml | CD4+ T cell count, anti-HIV antibody |
| | | levels, plasma viral RNA, |
| | | PBMC viral DNA |
| Day 3 | 2 ml | CD4+ T count, plasma viral RNA |
| Day 7 | 5 ml | CD4+ T cell count, anti-HIV antibody |
| | | levels, plasma viral RNA, |
| | | PBMC viral DNA |
| Week 2 | 5 ml | CD4+ T cell count, anti-HIV antibody |
| | | levels, plasma viral RNA, |
| | | PBMC viral DNA |
| Week 3 | 5 ml | CD4+ T cell count, anti-HIV antibody |
| | | levels, plasma viral RNA, |
| | | PBMC viral DNA |
| Week 5 | 10 ml | CD4+ T cell count, anti-HIV antibody |
| | | levels, plasma viral RNA, |
| | | PBMC viral DNA, virus isolation |
| Week 7 | 10 ml | CD4 count, antibody, plasma viral |
| | | RNA, PBMC viral DNA, virus isolation |
| Months 2, | | The same blood collection volumes |
| 3, 4, 5, 6 | | drawn and assays performed |
| | | as described above |

The development of AIDS is observed by the onset of AIDS-associated disease symptoms or AIDS-like syndrome. AIDS-associated disease is evidenced by, e.g., a loss of CD4+ T cells, development of plasma viremia, pneumocystis carinii pneumonia, cryptococcal meningoencephalitis (CME), Trichuris trichuria infection, mycotic gastritis, other opportunistic infections, and/or neurological disorders.

The resulting animal model is useful for testing the efficacy of anti-HIV drugs and determining whether immune responses induced by HIV-1 proteins (including, e.g., HIV vaccines, such as HIV vaccines made from envelope proteins) can protect against either infection or disease induced by the virus.

### EXAMPLE 7

### Virus Isolation from Infected Monkeys

Virus is subsequently isolated from infected monkeys as described in Shibata et al., J. Infectious Disease 176:362-373 (1997), which is incorporated herein by reference in its entirety for all purposes. Briefly, one million PBMC from the infected monkey are depleted of CD8 positive cells using a magnetic bead selection method (e.g., Stanciu L A et al., J. Immunol. Met. 189: 107-115, which is incorporated herein by reference in its entirety for all purposes) and are mixed with 5 million PBMC prepared from an uninfected monkey, stimulated with PHA, and cultured for 4 weeks. Culture supernatants are collected twice a week and those strongly positive by HIV p24 antigen assay are stored at −80° C. The isolated virus(es) may be used for passage to a naïve monkeys (see Section 2 above). As shown in Table 3, virus isolation is performed by-weekly or monthly.

The purpose of passaging the viruses from monkey to monkey in vitro or via in vitro methods is to increase the pathogenicity of the shuffled chimeric viruses of the invention. Animal to animal passage has been used to generate a pathogenic strain of simian HIV (SHIV) (see, e.g., Joag et al., J. Virology 70:3189-3197 (1996); Reimann et al., J. Virology 70:6922-6928 (1996).

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated herein by reference in their entirety for all purposes to the same extent as if each individual publication, patent, patent application, and/or other document were individually indicated to be incorporated herein by reference in its entirety for all purposes.

SEQUENCE LISTING

| SEQ ID | Clone | |
|---|---|---|
| NO: | Name/Type | Sequence |
| SEQ ID | clone 1.4 | TGGAAGGGATTTATTACAGTGCAAGAAGACATAGAATCTTAGACATATAC |
| NO: 1 | DNA | TTAAAAAAGGAAGAAGGCATCATACCAGATTGGCAGGATTACACCTCAGG |
| | | ACCAGGAATTAGATACCCAAAGACATTTGGCTGGCTATGGAAATTAGTCC |
| | | CTGTAAATGTATCAGATGAGGCACAGGAGGATGAGGAGCATTATTTAATG |
| | | CATCCAGCTCAAACTTCCCAGTGGGATGACCCTTGGGGAGAGGTTCTAGC |
| | | ATGGAAGTTTGATCCAACTCTGGCCTACACTTATGAGGCATATGTTAGAT |
| | | ACCCAGAAGAGTTTGGAAGCAAGTCAGGCCTGTCAGAGGAAGAGGTTAAA |
| | | AGAAGGCTAACCGCAAGAGGCCTTCTTAACATGGCTGACAAGAAGGAAAC |
| | | TCGCTGAATTCGAGCTATCTACAGGGGACTTTCCGCTGGGGACTTTCCAG |
| | | GGAGGCGTGGCCTGGGCGGGACCGGGGAGTGGCGAGCCCTCAGATGCTGC |
| | | ATATAAGCAGCCGCTTTTGCCTGTACTGGGTCTCTCTAGTTAGACCAGAT |
| | | CTGAGCCTGGGAGCTCTCTGGCTAGCTGAGAACCCACTGCTTAAGCCTCA |
| | | ATAAAGCTTGCCTTGAGTGCTTTAAGTAGTGTGTGCCCGTCTGTTGTGTG |
| | | ACTCTGGTAACTAGAGATCCCTCAGACCATTTTAGTCAGTGTGGAAAATC |
| | | TCTAGCAGTGGCGCCCGAACAGGGACCGGAAAGCGAAAGAGAAACCAGAG |
| | | AAGCTCTCTCGACGCAGGACTCGGCTTGCTGAAGCGCGCACGGCAAGCGG |
| | | CGAGGGGCAGCGACCGGTGAGTACGCTAAAAATTTTGACTAGCGGAGGCT |
| | | AGAAGGAGAGAGATGGGTGCGAGAGCGTCAGTATTAAGCGGGGGAAAATT |
| | | GGATGCATGGGAAAAAATTCGGTTACGGCCAGGAGGAAAGAAAAAATATA |
| | | GACTAAAACATCTAGTATGGGCAAGCAGGGAGCTAGAACGATTTGCACTT |
| | | AATCCTGGCCTTTTAGAGACATCAGATGGCTGTAAACAAATAATAGGACA |
| | | GCTACAACCAGCTATCCGGACAGGATCAGAAGAACTTAGATCATTATTTA |
| | | ATACAGTAGCAACCCTCTATTGTGTACATGAAAGGATAGAGGTAAAAGAC |
| | | ACCAAGGAAGCTTTAGAGAAGATAGAGGAAGAGCAAAACAAAAGTAAGAA |
| | | AAAAGCACAGCAAGCAGCAGCTGACACAGGACACAGCAATCAGGTCAGCC |
| | | AAAATTACCCTATAGTGCAGAACATCCAGGGGCAAATGGTACATCAGGCC |
| | | CTATCACCTAGAACTTTAAATGCGTGGGTAAAAGTAGTAGAAGAGAAGGC |
| | | TTTTAGCCCAGAAGTAATACCCATGTTTTCAGCATTATCAGAAGGAGCCA |
| | | CCCCACAAGATTTAAACACCATGCTAAACACAGTGGGGGGACATCAAGCA |
| | | GCCATGCAAATGTTAAAAGAGACCATCAATGAGGAAGCTGCAGAATGGGA |
| | | TAGAGTGCATCCAGTGCATGCAGGGCCTATTGCACCAGGCCAGATGAGAG |
| | | AACCAAGGGGAAGTGACATAGCAGGAACTACTAGTACCCTTCAGGAACAA |
| | | ATAGGATGGATGACACATAATCCACCTATCCCAGTAGGAGAAATCTATAA |
| | | AAGATGGATAATCCTGGGATTAAATAAAATAGTAAGAATGTATAGCCCTA |
| | | CCAGCATTCTGGACATAAGACAAGGACCAAAGGAACCCTTTAGAGACTAT |
| | | GTAGACCGGTTCTATAAAACCCTAAGAGCCGAGCAAGCTACACAGGAGGT |
| | | AAAAAATTGGATGACAGAAACCTTGTTGGTCCAAAATGCGAACCCAGATT |
| | | GTAAAACTATTTTAAAAGCATTGGGACCAGCAGCCACACTAGAAGAAATG |
| | | ATGACAGCATGTCAGGGAGTGGGGGGACCCGGCCATAAAGCAAGAGTTTT |
| | | GGCTGAAGCAATGAGCCAAGTAACAAATTCAGCTACCATAATGATGCAGA |
| | | GAGGCAATTTTAGGAACCAAAGAAAAACTGTTAAGTGTTTCAATTGTGGC |
| | | AAAGAAGGGCACATAGCCAAAAATTGCAGGGCTCCTAGGAAAAAGGGCTG |
| | | TTGGAAATGTGGAAAGGAAGGACACCAAATGAAAGATTGTACTGAGAGAC |
| | | AGGCTAATTTTTTAGGGAAGATCTGGCCTTCCCACAAGGGAAGGCCAGGA |
| | | AATTTTCTTCAGAGCAGACCAGAGCCAACAGCCCCATCAGAAGAGAGCGT |
| | | CAAGTTTGGAGAAGAGACAACAACTCCCTCTCAGAAGCAGGAGCCGATAG |
| | | ACAAGGAACTGTATCCTTTAACTTCCCTCAGATCACTCTTTGGCAACGAC |
| | | CCCTCGTCACAATAAAGATAGGGGGGCAACTAAAGGAAGCTCTATTAGAT |
| | | ACAGGAGCAGATGATACAGTATTAGAAGACATGGATTTGCCAGGAAGATG |
| | | GAAACCAAAAATGATAGGGGGAATTGGAGGTTTTATCAAAGTAAGACAGT |
| | | ATGATCAGATACCCATAGATATCTGTGGACATAAAGCTGTAGGTACAGTA |
| | | TTAGTAGGACCTACACCTGTCAACATAATTGGAAGAAATCTGTTGACTCA |
| | | GATTGGTTGCACTTTAAATTTTCCCATTAGTCCTATTGAAACTGTACCAG |
| | | TAAAATTAAAGCCAGGAATGGATGGCCCAAAAGTCAAACAATGGCCATTG |
| | | ACAGAAGAAAAAATAAAAGCATTAGTAGAAATTTGTACAGAAATGGAAAA |
| | | GGAAGGAAAGATTTCAAAAATTGGGCCTGAAAATCCATACAATACTCCAG |
| | | TATTTGCCATAAAGAAAAAAGACAGTACTAAATGGAGAAAATTAGTAGAT |
| | | TTCAGAGAACTTAATAGGAAAACTCAAGACTTCTGGGAAGTTCAATTAGG |
| | | AATACCACATCCCGCAGGGTTAAAAAAGAAAAAATCAGTAACAGTACTGG |
| | | ATGTGGGTGATGCATATTTTTCAGTTCCCTTAGATAAAGACTTCAGGAAG |
| | | TATACTGCATTTACCATACCTAGTATAAACAATGAGACACCAGGGATTAG |
| | | ATATCAGTACAATGTGCTTCCACAGGGATGGAAAGGATCACCAGCAATAT |
| | | TCCAAAGTAGCATGACAAAAACCTTAGAGCCTTTTAGAAAACAAAATCCA |
| | | GACATAATTATCTATCAATACATGGATGATTTGTATGTAGGATCTGACTT |
| | | AGAAATAGGGCAGCATAGAACAAAAATAGAGGAACTGAGACAACATCTGT |
| | | TGAAGTGGGGATTTACCACACCAGACAAAAAACATCAGAAAGAACCTCCA |
| | | TTCCTTTGGATGGGTTATGAACTCCATCCTGATAAATGGACAGTACAGCC |
| | | TATAGTGCTGCCAGAAAAAGACAGCTGGACTGTCAATGACATACAGAAGT |
| | | TAGTGGGAAAATTAAATTGGGCAAGTCAAATTTATGCAGGGATTAAAGTA |
| | | AAGCAATTATGTAAACTCCTTAGGGGAACCAAAGCACTTACAGAAGTAAT |
| | | ACCACTAACAAAAGAAGCAGAGCTAGAACTGGCAGAAAACAGGGAGATTC |
| | | TAAAGGAACCAGTACATGGAGTGTATTATGACCCATCAAAAGACTTAATA |
| | | GTAGAAATACAGAAGCAGGGGCAAGGCCAATGGACATATCAAATTTTTCA |
| | | AGAGCCATTTAAAAATCTGAAAACAGGAAAATATGCAAAAACGAGGGGTG |
| | | CCCACACTAATGATGTAAAACAATTAACAGAGGCAGTGCAAAAAATAGCC |
| | | AATGAAAGCATAGTAATATGGGGAAAGATTCCTAAATTTAAATTACCCAT |
| | | ACAAAAAGAAACATGGGAAACATGGTGGACAGAGTATTGGCAAGCCACCT |
| | | GGATTCCTGAGTGGGAGTTTGTCAATACCCCTCCCTTAGTGAAATTATGG |
| | | TACCAGTTAGAAAAAGAACCCATAGTAGGAGCAGAAACTTTCTATGTAGA |
| | | TGGGGCAGCTAACAGGGAGACTAAATTAGGAAAAGCAGGATATGTTACTA |
| | | GCAGAGGAAGGCAAAAAGTTGTCTCCCTAACAGACACAACAAATCAGAAA |
| | | ACTGAGTTACAAGCAATTCACCTAGCTTTGCAGGATTCAGGATTAGAAGT |
| | | AAACATAGTAACAGACTCACAATATGCATTAGGAATCATTCAAGCACAAC |
| | | CAGATAAAAGTGAATCAGAGTTAGTCAGTCAAATAATAGAACAGCTAATA |
| | | AAAAAGGAAAAAGTCTACCTGGCATGGGTACCAGCACACAAAGGAATTGG |
| | | AGGAAATGAACAGGTAGATAAATTAGTCAGTGCTGGAATCAGGAGAGTAC |
| | | TATTTCTAGATGGAATAGAGAAGGCCCAAGAAGAACATGAGAAATATCAT |
| | | AATAATTGGAGAGCAATGGCTAGTGAATTTAACCTGCCAGCTGTAGTAGC |
| | | AAAAGAGATAGTAGCCTGCTGTGATAAGTGCCAGGTAAAAGGAGAAGCCA |
| | | TGCATGGACAAGTAGACTGCAGTCCAGGAATATGGCAACTAGATTGTACA |
| | | CATTTAGAAGGAAAAGTTATCCTGGTAGCAGTTCATGTAGCCAGTGGATA |
| | | TATAGAAGCAGAGGTTATTCCAGCAGAGACAGGACAGGAAACAGCATACT |
| | | TTATTTTAAAATTAGCAGGAAGATGGCCAGTAAAAACAATACATACAGAC |
| | | AATGGCAGTAATTTCACCAGTACTACGGTTAAGGCCGCCTGTTGGTGGGC |
| | | AGGGATCAAGCAGGAATTTGGCATTCCCTACAATCCCCAAAGTCAAGGAG |
| | | TAGTAGAATCTATGAATAAAGAATTAAAGAAAATTATAGAACAAGTAAGA |
| | | GATCAGGCTGAACATCTTAAGACAGCAGTACAAATGGCAGTATTCATTCA |
| | | CAATTTTAAAAGAAAAGGGGGGATTGGGGGGTACAGTGCAGGGGAAAGAA |
| | | TAGTAGACATAATAGCATCAGACATACAAACTAAAGAACTACAAAAACAA |
| | | ATCACAAAAATTCAAAATTTTCGGGTTTATTACAGGGACAGCAGAGATCC |
| | | ACTTTGGAAAGGACCAGCAAAGCTTCTTTGGAAAGGTGAAGGGGCAGTAG |
| | | TAATACAAGATAAGAGTGACATAAAAGTAGTGCCAAGAAGAAAAGCAAAG |
| | | ATTATCAGGGATTATGGAAAACAGATGGCAGGTGATGATTGTGTGGCAAG |
| | | TAGACAGGATGAGGATTAGAACATGGAAAAGTTTAGTAAAACACCATATG |
| | | TATGTTTCAAAGAAAGCTAAGGGATGGTTTTATAGACATCACTATGAAAG |
| | | CACTCATCCAAGAATAAGTTCAGAAGTACATATCCCACTAGGGGATGCTA |
| | | GCTTGGTAGTAACAACATATTGGGGTCTACATACACGAGAAAGAGACTGG |
| | | CATTTGGGTCAGGGAGTCTCCATAGAATGGAGGAAAAGGAGATACAGCAC |
| | | ACAAGTAGACCCTGACCTAGCAGACCAACTAATTCATCTGTACTACTTTG |
| | | ATTGTTTTTCAGAATCTGCTATAAGAAATGCCATATTAGGACATAGAGTT |
| | | AGTCCTAGGTGTGAATATCAAGCAGGACATAACAAGGTAGGATCTCTACA |
| | | GTACTTGGCACTAGCAGCATTAGTAACACCAAGAAAGATAAAGCCACCTT |
| | | TGCCTAGTGTTGCGAAACTGACAGAGGACAGATGGAACAAGTCCCACAAG |
| | | ACCAAGGGCCACAGAGGGAGCCATACAATGAATGGACACTAAAGCTTTTA |
| | | GAGGAGCTTAAGAATGAAGCTGTCAGACATTTCCCTAGACCATGGCTTCA |
| | | TGGCCTAGGGCAATATATCTATGAAACTTATGAGGATACTTGGGCAGGAG |
| | | TGGAAGCCATAATAAGAATTCTGCAACAATTGCTGCTTATTCATTTCAGA |
| | | ATTGGGTGTCAACATAGCAGAATAGGCATTATTCGACAGAGGAGAACAAG |
| | | AAATGGAGCCAGTAGATCCTAGACTAGAGCCCTGGAAGCATCCAGGAAGT |
| | | CAGCCTAAGACTGCCTGTACCAATTGCTATTGCAAAAAGTGTTGCTTGCA |
| | | TTGCCAAGTTTGCTTCATAACAAAAGGCTTAGGCATCTCCTATGGCAGGA |
| | | AGAAGCGGAAAAAGCGACGAAGATCTCCTCAACACAGTCAGACTGATCAA |
| | | GCTTCTCTATCAAAGCAGTAAGTAGTACATGTAATGCAACCTTTAGTAAT |
| | | ATTAGCAATAGTAGCATTAGTAGTAGCACTAATAATAGTCATAGTTGTAT |
| | | GGTCCATTGTATTAATAGAATATAGAAAAATATTAAGACAAAAGAAAATA |
| | | GACAGGTTAATTGATAGAATAAGAGAAAAAGCAGAAGACAGTGGCAATGA |
| | | GAGTGATGGGGATCAGGAAGAATTATCAGCACTTGTGGAAAGGGGGCACC |
| | | TTGCTCCTTGGGATATTGATGATCTGTAGTGCTGCAGAACAATTGTGGGT |
| | | CACAGTCTATTATGGGGTACCTGTGTGGAAAGAAGCAAACACCACTCTAT |
| | | TTTGTGCATCAGATGCTAAGGCATATGATACAGAGGTACATAATGTTTGG |
| | | GCCACACATGCCTGTGTACCCACAGACCCCAACCCACAAGAAATACTATT |
| | | GGAAAATGTGACAGAAGATTTTAACATGTGGAAAAATAACATGGTAGAAC |
| | | AGATGCATGAGGATATAATCAGTTTATGGGATCAAAGTCTAAAGCCATGT |
| | | GTAAAATTAACCCCACTCTGTGTTACTTTACATTGCACTGATTTGAAGAA |
| | | TGGTACTAATTTGAAGAATGGTACTAAAATCATTGGGAAATCAATAAGAG |
| | | GAGAAATAAAAAACTGCTCTTTCAATGTCACCAAAAACATAATAGATAAG |
| | | GTGAAAAAAGAATATGCGCTTTTCTATAGACATGATGTAGTACCAATAGA |
| | | TAGGAATATTACTAGCTATAGGTTAATAAGTTGTAACACCTCAACCCTTA |
| | | CACAGGCCTGTCCAAAGGTATCCTTTGAGCCAATTCCCATACATTATTGT |
| | | GCCCCGGCTGGTTTTGCGATTCTAAAATGTAAAGATAAGAAGTTCAATGG |
| | | AACGGGACCATGTACAAATGTCAGTACAGTACAATGTACACATGGAATTA |
| | | GGCCAGTAGTATCAACTCAACTGCTGTTAAATGGAAGTCTAGCAGAAGAA |
| | | GAGGTAGTAATTAGATCTAGCAATTTCACGGACAATGCTAAAATCATAAT |
| | | AGTACAGCTGAATGAAACTGTAGAAATTAATTGTACAAGACCCAACAACA |
| | | ATACAAGAAAAGGGATAACTCTAGGACCAGGGAGAGTATTTTATACAACA |
| | | GGAAAAATAGTAGGAGATATAAGAAAAGCACATTGTAACATTAGTAAAGT |
| | | AAAATGGCATAACACTTTAAAAAGGGTAGTTAAAAAATTAAGAGAAAAAT |
| | | TTGAAAATAAAACAATAATCTTTAATAAATCCTCAGGGGGGGACCCAGAA |
| | | ATTGTAATGCACAGCTTTAATTGTGGAGGGGAATTTTTCTACTGTAATAC |
| | | AAAAAAACTGTTTAATAGTACTTGGAATGGTACTGAAGGGTCATATAACA |
| | | TTGAAGGAAATGACACTATCACACTCCCATGCAGAATAAAACAAATTATA |
| | | AACATGTGGCAGGAAGTAGGAAAAGCAATGTATGCCCCTCCCATCAGTGG |
| | | ACAAATTTGGTGCTCATCAAATATTACAGGGCTGCTACTAACAAGAGATG |
| | | GTGGTAAGAACAGCAGCACCGAAATCTTCAGACCTGGAGGAGGAGATATA |
| | | AGGGACAATTGGAGAAGTGAATTATATAAATATAAAGTAGTAAGAGTTGA |
| | | ACCATTAGGAATAGCACCCACCAAGGCAAAAAGAAGAGTGGTGCAGAGAG |
| | | AAAAAAGAGCAGTGGGAATAGGAGCTGTGTTCCTTGGGTTCTTGGGAGCA |
| | | GCAGGAAGCACTATGGGCGCAGCGTCAATAACGCTGACGGTACAGGCCAG |
| | | ACAATTATTGTCTGGTATAGTGCAACAGCAGAACAATTTGCTGAGGGCTA |
| | | TTGAAGCGCAACAGCATATGTTGCAACTCACAGTCTGGGGCATCAAGCAG |
| | | CTCCAGGCAAGAGTCCTGGCTGTGGAAAGATACCTACAGGATCAACAGCT |
| | | CCTGGGGATTTGGGGTTGCTCTGGAAAACTCATCTGCACCACTACTGTGC |
| | | CTTGGAATACTAGTTGGAGTAATAAATCTCTGGATACAATTTGGGGTAAC |
| | | ATGACCTGGATGCAGTGGGAAAAAGAAATTAACAATTACACAGGCTTAAT |
| | | ATACAACTTGATTGAAGAATCGCAGAACCAACAAGAAAAGAATGAACAAG |
| | | AATTATTGGCATTAGATAAATGGGCAAGTTTGTGGAATTGGTTTAACATA |
| | | TCAAACTGGCTGTGGTATATAAAAATATTCATAATGATAGTAGGAGGCTT |
| | | GATAGGTTTAAGAATAGTTTTCAGTGTACTTTCTATAGTGAATAGAGTTA |
| | | GGCAGGGATACTCACCATTATCGTTTCAGACCCGCTTCCCAGCCTCGAGG |
| | | GGACCCGACAGGCCCGAAGGAATCGAAGAAGAAGGTGGAGACAGAGACAG |
| | | AGACAGATCCAGTCCATTAGTGGATGGATTCTTAGCAATCATCTGGGTCG |
| | | ACCTGCGGAGCCTGTTCCTCTTCAGCTACCACCGCTTGAGAGACTTACTC |
| | | TTGATTGTAACGAGGATTGTGGAACTTCTGGGACGCAGGGGGTGGGAACT |
| | | CCTCAAATACTTGTGGAATCTCCTGCAGTATTGGAGTCAGGAACTAAAGA |
| | | ATAGTGCTGTTAGCTTGCTTAACGCCACAGCCATAGCAGTAGGTGAGGGA |
| | | ACAGATAGAATTATAGAAATATTACAAAGAGCTGGTAGAGCTATTCTCAA |
| | | CATACCTACGAGAATAAGACAGGGCTTAGAAAGGGCTTTGCTATAAGCTT |
| | | ATGGGTGGAGCTATTTCCATGAGGCGGTCCAGGCCGTCTGGAAATCTGTA |
| | | CGAGAGACTCTTGCGGGCGCGTGGGGAGACTTATGGAAAACTCTTAGGAG |
| | | AGGTAAAAGATGGATACTCGCAATCCCCAGGAGGATTAGACAAGGGCTTG |
| | | AGCTCACTCTCTTGTGAGGGACAAAAATACAATCAGGGACAGTATATGAA |
| | | TACTCCATGGAGAAACCCAGCTAAAGAGAGAGAAAAATTAGCATACAGAA |
| | | AACAAAATATGGATGATATAGATAAGGAAGATGATGACTTGGTAGGGGTA |
| | | TCAGTGAGGCCAAAAGTTCCCCTAAGAACAATGAGTTACAAATTGGCAAT |
| | | AGACATGTCTCATTTTATAAAAGAAAAGGGGGGACTGGAAGGGATTTATT |
| | | ACAGTGCAAGAAGACATAGAATCTTAGACATATACTTAAAAAAGGAAGAA |
| | | GGCATCATACCAGATTGGCAGGATTACACCTCAGGACCAGGAATTAGATA |
| | | CCCAAAGACATTTGGCTGGCTATGGAAATTAGTCCCTGTAAATGTATCAG |
| | | ATGAGGCACAGGAGGATGAGGAGCATTATTTAATGCATCCAGCTCAAACT |
| | | TCCCAGTGGGATGACCCTTGGGGAGAGGTTCTAGCATGGAAGTTTGATCC |
| | | AACTCTGGCCTACACTTATGAGGCATATGTTAGATACCCAGAAGAGTTTG |
| | | GAAGCAAGTCAGGCCTGTCAGAGGAAGAGGTTAAAAGAAGGCTAACCGCA |
| | | AGAGGCCTTCTTAACATGGCTGACAAGAAGGAAACTCGCTGAATTCGAGC |
| | | TATCTACAGGGGACTTTCCGCTGGGGACTTTCCAGGGAGGCGTGGCCTGG |
| | | GCGGGACCGGGGAGTGGCGAGCCCTCAGATGCTGCATATAAGCAGCCGCT |
| | | TTTGCCTGTACTGGGTCTCTCTAGTTAGACCAGATCTGAGCCTGGGAGCT |
| | | CTCTGGCTAGCTGAGAACCCACTGCTTAAGCCTCAATAAAGCTTGCCTTG |
| | | AGTGCTTTAAGTAGTGTGTGCCCGTCTGTTGTGTGACTCTGGTAACTAGA |
| | | GATCCCTCAGACCATTTTAGTCAGTGTGGAAAATCTCTAGCA |
| SEQ ID | clone | TGGAAGGGATTTATTACAGTGCAAGAAGACATAGAATCTTAGACATATAC |
| NO: 2 | P10.26 DNA | TTAGAAAAGGAAGAAGGCATCATACCAGATTGGCAGGATTACACCTCAGG |
| | | ACCAGGAATTAGATACCCAAAGACATTTGGCTGGCTATGGAAATTAGTCC |
| | | CTGTAAATGTATCAGATGAGGCACAGGAGGATGAGGAGCATTATTTAATG |
| | | CATCCAGCTCAAACTTCCCAGTGGGATGACCCTTGGGGAGAGGTTCTAGC |
| | | ATGGAAGTTTGATCCAACTCTGGCCTACACTTATGAGGCATATGTTAGAT |
| | | ACCCAGAAGAGTTTGGAAGCAAGTCAGGCCTGTCAGAGGAAGAGGTTAAA |
| | | AGAAGGCTAACCGCAAGAGGCCTTCTTAACATGGCTGACAAGAAGGAAAC |
| | | TCGCTGAATTCGAGCTATCTACAGGGGACTTTCCGCTGGGGACTTTCCAG |
| | | GGAGGCGTGGCCTGGGCGGGACCGGGGAGTGGCGAGCCCTCAGATGCTGC |
| | | ATATAAGCAGCCGCTTTTGCCTGTACTGGGTCTCTCTAGTTAGACCAGAT |
| | | CTGAGCCTGGGAGCTCTCTGGCTAGCTGAGAACCCACTGCTTAAGCCTCA |
| | | ATAAAGCTTGCCTTGAGTGCTTTAAGTAGTGTGTGCCCGTCTGTTGTGTG |
| | | ACTCTGGTAACTAGAGATCCCTCAGACCATTTTAGTCAGTGTGGAAAATC |
| | | TCTAGCAGTGGCGCCCGAACAGGGACCGGAAAGCGAAAGAGAAACCAGAG |
| | | AAGCTCTCTCGACGCAGGACTCGGCTTGCTGAAGCGCGCACGGCAAGCGG |
| | | CGAGGGGCAGCGACCGGTGAGTACGCTAAAAATTTTGACTAGCGGAGGCT |
| | | AGAAGGAGAGAGATGGGTGCGAGAGCGTCAGTATTAAGCGGGGGAAAATT |
| | | GGATGCATGGGAAAAAATTCGGTTACGGCCAGGAGGAAAGAAAAAATATA |
| | | GACTAAAACATCTAGTATGGGCAAGCAGGGAGCTAGAACGATTTGCACTT |
| | | AATCCTGGCCTTTTAGAGACATCAGATGGCTGTAAACAAATAATAGGACA |
| | | GCTACAACCAGCTATCCGGACAGGATCAGAAGAACTTAGATCATTATTTA |
| | | ATACAGTAGCAACCCTCTATTGTGTACATGAAAGGATAAAGGTAAAAGAC |
| | | ACCAAGGAAGCTTTAGAGAAGATAGAGGAAGAGCAAAACAAAAGTAAGAA |
| | | AAAAGCACAGCAAGCAGCAGCTGACACAGGACACAGCAATCAGGTCAGCC |
| | | AAAATTACCCTATAGTGCAGAACATCCAGGGGCAAATGGTACATCAGGCC |
| | | CTATCACCTAGAACTTTAAATGCGTGGGTAAAAGTAGTAGAAGAGAAGGC |
| | | TTTTAGCCCAGAAGTAATACCCATGTTTTCAGCATTATCAGAAGGAGCCA |
| | | CCCCACAAGATTTAAACACCATGCTAAACACAGTGGGGGGACATCAAGCA |
| | | GCCATGCAAATGTTAAAAGAGACCATCAATGAGGAAGCTGCAGAATGGGA |
| | | TAGAGTGCATCCAGTGCATGCAGGGCCTATTGCACCAGGCCAGATGAGAG |
| | | AACCAAGGGGAAGTGACATAGCAGGAACTACTAGTACCCTTCAGGAACAA |
| | | ATAGGATGGATGACACATAATCCACCTATCCCAGTAGGAGAAATCTATAA |
| | | AAGATGGATAATCCTGGGATTAAATAAAATAGTAAGAATGTATAGCCCTA |
| | | CCAGCATTCTGGACATAAGACAAGGACCAAAGGAACCCTTTAGAGACTAT |
| | | GTAGACCGGTTCTATAAAACCCTAAGAGCCGAGCAAGCTACACAGGAGGT |
| | | AAAAAATTGGATGACAGAAACCTTGTTGGTCCAAAATGCGAACCCAGATT |
| | | GTAAAACTATTTTAAAAGCATTGGGACCAGCAGCCACACTAGAAGAAATG |
| | | ATGACAGCATGTCAGGGAGTGGGAGGACCCGGCCATAAAGCAAGAGTTTT |
| | | GGCTGAAGCAATGAGCCAAGTAACAAATTCAGCTACCATAATGATGCAGA |
| | | GAGGCAATTTTAGGAACCAAAGAAAAACTGTTAGGTGTTTCAATTGTGGC |
| | | AAAGAAGGGCACATAGCCAAAAATTGCAGGGCTCCTAGGAAAAAGGGCTG |
| | | TTGGAAATGTGGAAAGGAAGGACACCAAATGAAAGATTGTACTGAGAGAC |
| | | AGGCTAATTTTTTAGGGAAGATCTGGCCTTCCCACAAGGGAAGGCCAGGA |
| | | AATTTTCCTCAGAGCAGACCAGAGCCAACAGCCCCATCAGAAGAGAGCGT |
| | | CAAGTTTGGAGAAGAGACAACAACTCCCTCTCAGAAGCAGGAGCCGATAG |
| | | ACAAGGAACTGTATCCTTTAACTTCCCTCAGATCACTCTTTGGCAACGAC |
| | | CCCTCGTCACAATAAAGATAGGGGGGCAACTAAAGGAAGCTCTATTAGAT |
| | | ACAGGAGCAGATGATACAGTATTAGAAGACATGGATTTGCCAGGAAGATG |
| | | GAAACCAAAAATGATAGGGGGAATTGGAGGTTTTATCAAAGTAAGACAGT |
| | | ATGATCAGATACCCATAGATATCTGTGGACATAAAGCTGTAGGTACAGTA |
| | | TTAGTAGGACCTACACCTGTCAACATAATTGGAAGAAATCTGTTGACTCA |
| | | GATTGGTTGCACTTTAAATTTTCCCATTAGTCCTATTGAAACTGTACCAG |
| | | TAAAATTAAAGCCAGGAATGGATGGCCCAAAAGTCAAACAATGGCCATTG |
| | | ACAGAAGAAAAAATAAAAGCATTAGTAGAAATTTGTACAGAAATGGAAAA |
| | | GGAAGGAAAGATTTCAAAAATTGGGCCTGAAAATCCATACAATACTCCAG |
| | | TATTTGCCATAAAGAAAAAAGACAGTACTAAATGGAGAAAATTAGTAGAT |
| | | TTCAGAGAACTTAATAGGAAAACTCAAGACTTCTGGGAAGTTCAATTAGG |
| | | AATACCACATCCCGCAGGGTTAAAAAAGAAAAAATCAGTAACAGTACTGG |
| | | ATGTGGGTGATGCATATTTTTCAGTTCCCTTAGATAAAGACTTCAGGAAG |
| | | TATACTGCATTTACCATACCTAGTATAAACAATGAGACACCAGGGATTAG |
| | | ATATCAGTACAATGTGCTTCCACAGGGATGGAAAGGATCACCAGCAATAT |
| | | TCCAAAGTAGCATGACAAAAACCTTAGAGCCTTTTAGAAAACAAAATCCA |
| | | GACATAATTATCTATCAATACATGGATGATTTGTATGTAGGATCTGACTT |
| | | AGAAATAGGGCAGCATAGAACAAAAATAGAGGAACTGAGACAACATCTGT |
| | | TGAAGTGGGGATTTACCACACCAGACAAAAAACATCAGAAAGAACCTCCA |
| | | TTCCTTTGGATGGGTTATGAACTCCATCCTGATAAATGGACAGTACAGCC |
| | | TATAGTGCTGCCAGAAAAAGACAGCTGGACTGTCAATGACATACAGAAGT |
| | | TAGTGGGAAAATTGAATTGGGCAAGTCAAATTTATGCAGGGATTAAAGTA |
| | | AAGCAATTATGTAAACTCCTTAGGGGAACCAAAGCACTTACAGAAGTAAT |
| | | ACCACTAACAAAAGAAGCAGAGCTAGAACTGGCAGAAAACAGGGAGATTC |
| | | TAAAGGAACCAGTACATGGAGTGTATTATGACCCATCAAAAGACTTAATA |
| | | GTAGAAATACAGAAGCAGGGGCAAGGCCAATGGACATATCAAATTTTTCA |
| | | AGAGCCATTTAAAAATCTGAAAACAGGAAAATATGCAAAAACGAGGGGTG |
| | | CCCACACTAATGATGTAAAACAATTAACAGAGGCAGTGCAAAAAATAGCC |
| | | AATGAAAGCATAGTAATATGGGGAAAGATTCCTAAATTTAAATTACCCAT |
| | | ACAAAAAGAAACATGGGAAACATGGTGGACAGAGTATTGGCAAGCCACCT |
| | | GGATTCCTGAGTGGGAGTTTGTCAATACCCCTCCCTTAGTGAAATTATGG |
| | | TACCAGTTAGAAAAAGAACCCATAGTAGGAGCAGAAACTTTCTATGTAGA |
| | | TGGGGCAGCTAACAGGGAGACTAAATTAGGAAAAGCAGGATATGTTACTA |
| | | GCAGAGGAAGGCAAAAAGTTGTCTCCCTAACAGACACAACAAATCAGAAA |
| | | ACTGAGTTACAAGCAATTCACCTAGCTTTGCAGGATTCAGGATTAGAAGT |
| | | AAACATAGTAACAGACTCACAATATGCATTAGGAATCATTCAAGCACAAC |
| | | CAGATAAAAGTGAATCAGAGTTAGTCAGTCAAATAATAGAACAGCTAATA |
| | | AAAAAGGAAAAAGTCTACCTGGCATGGGTACCAGCACACAAAGGAATTGG |
| | | AGGAAATGAACAGGTAGATAAATTAGTCAGTGCTGGAATCAGGAGAGTAC |
| | | TATTTCTAGATGGAATAGAGAAGGCCCAAGAAGAACATGAGAAATATCAT |
| | | AATAATTGGAGAGCAATGGCTAGTGAATTTAACCTGCCAGCTGTAGTAGC |
| | | AAAAGAGATAGTAGCCTGCTGTGATAAGTGCCAGGTAAAAGGAGAAGCCA |
| | | TGCATGGACAAGTAGACTGCAGTCCAGGAATATGGCAACTAGATTGTACA |
| | | CATTTAGAAGGAAAAGTTATCCTGGTAGCAGTTCATGTAGCCAGTGGATA |
| | | TATAGAAGCAGAGGTTATTCCAGCAGAGACAGGACAGGAAACAGCATACT |
| | | TTATTTTAAAATTAGCAGGAAGATGGCCAGTAAAAACAATACATACAGAC |
| | | AATGGCAGTAATTTCACCAGTACTACGGTTAAGGCCGCCTGTTGGTGGGC |
| | | AGGGATCAAGCAGGAATTTGGCATTCCCTACAATCCCCAAAGTCAAGGAG |
| | | TAGTAGAATCTATGAATAAAGAATTAAAGAAAATTATAGAACAAGTAAGA |
| | | GATCAGGCTGAACATCTTAAGACAGCAGTACAAATGGCAGTATTCATTCA |
| | | CAATTTTAAAAGAAAAGGGGGGATTGGGGGGTACAGTGCAGGGGAAAGAA |
| | | TAGTAGACATAATAGCATCAGACATACAAACTAAAGAACTACAAAAACAA |
| | | ATTACAAAAATTCAAAATTTTCGGGTTTATTACAGGGACAGCAGAGATCC |
| | | ACTTTGGAAAGGACCAGCAAAGCTTCTTTGGAAAGGTGAAGGGGCAGTAG |
| | | TAATACAAGATAAGAGTGACATAAAAGTAGTGCCAAGAAGAAAAGCAAAG |
| | | ATTATCAGGGATTATGGAAAACAGATGGCAGGTGATGATTGTGTGGCAAG |
| | | TAGACAGGATGAGGATTAGAACATGGAAAAGTTTAGTAAAACACCATATG |
| | | TATGTTTCAAAGAAAGCTAAGGGATGGTTTTATAGACATCACTATGAAAG |
| | | CACTCATCCAAGAATAAGTTCAGAAGTACATATCCCACTAGGGGATGCTA |
| | | GCTTGGTAGTAACAACATATTGGGGTCTACATACAGGAGAAAGAGACTGG |
| | | CATTTGGGTCAGGGAGTCTCCATAGAATGGAGGAAAAGGAGATACAGCAC |
| | | ACAAGTAGACCCTGACCTAGCAGACCAACTAATTCATCTGTACTACTTTG |
| | | ATTGTTTTTCAGAATCTGCTATAAGAAATGCCATATTAGGACATAGAGTT |
| | | AGTCCTAGGTGTGAATATCAAGCAGGACATAACAAGGTAGGATCTCTACA |
| | | GTACTTGGCACTAGCAGCATTAGTAACACCAAGAAAGATAAAGCCACCTT |
| | | TGCCTAGTGTTGCGAAACTGACAGAGGACAGATGGAACAAGTCCCACAAG |
| | | ACCAGGGGCCACAGAGGGAGCCATACAATGAATGGACACTAAAGCTTTTA |
| | | GAGGAGCTTAAGAATGAAGCTGTCAGACATTTCCCTAGACCATGGCTTCA |
| | | TGGCCTAGGGCAATATATCTATGAAACTTATGAGGATACTTGGGCAGGAG |
| | | TGGAAGCCATAATAAGAATTCTGCAACAATTGCTGCTTATTCATTTCAGA |
| | | ATTGGGTGTCAACATAGCAGAATAGGCATTATTCGACAGAGGAGAACAAG |
| | | AAATGGAGCCAGTAGATCCTAGACTAGAGCCCTGGAAGCATCCAGGAAGT |
| | | CAGCCTAAGACTGCCTGTACCAATTGCTATTGCAAAAAGTGTTGCTTGCA |
| | | TTGCCAAGTTTGCTTCATAACAAAAGGCTTAGGCATCTCCTATGGCAGGA |
| | | AGAAGCGGAAAAAGCGACGAAGATCTCCTCAACACAGTCAGACTGATCAA |
| | | GCTTCTCTATCAAAGCAGTAAGTAGTACATGTAATGCAACCTTTAGTAAT |
| | | ATTAGCAATAGTAGCATTAGTAGTAGCACTAATAATAGTCATAGTTGTAT |
| | | GGTCCATTGTATTAATAGAATATAGAAAAATATTAAGACAAAAGAAAATA |
| | | GACAGGTTAATTGATAGAATAAGAGAAAAAGCAGAAGACAGTGGCAATGA |
| | | GAGTGATGGGGATCAGGAAGAATTATCAGCACTTGTGGAAAGGGGGCACC |
| | | TTGCTCCTTGGGATATTGATGATCTGTAGTGCTGCAGAACAATTGTGGGT |
| | | CACAGTCTATTATGGGGTACCTGTGTGGAAAGAAGCAAACACCACTCTAT |
| | | TTTGTGCATCAGATGCTAAGGCATATGATACAGAGGTACATAATGTTTGG |
| | | GCCACACATGCCTGTGTACCCACAGACCCCAACCCACAAGAAATACTATT |
| | | GGAAAATGTGACAGAAGATTTTAACATGTGGAAAAATAACATGGTAGAAC |
| | | AGATGCATGAGGATATAATCAGTTTATGGGATCAAAGTCTAAAGCCATGT |
| | | GTAAAATTAACCCCACTCTGTGTTACTTTACATTGCACTGATTTGAAGAA |
| | | TGGTACTAATTTGAAGAATGGTACTAAAATCATTGGGAAATCAATAAGAG |
| | | GAGAAATAAAAAACTGCTCTTTCAATGTCACCAAAAACATAATAGATAAG |
| | | GTGAAAAAAGAATATGCGCTTTTCTATAGACATGATGTAGTACCAATAGA |
| | | TAGGAATATTACTAGCTATAGGTTAATAAGTTGTAACACCTCAACCCTTA |
| | | CACAGGCCTGTCCAAAGGTATCCTTTGAGCCAATTCCCATACATTATTGT |
| | | GCCCCGGCTGGTTTTGCGATTCTAAAATGTAAAGATAAGAAGTTCAATGG |
| | | AACGGGACCATGTACAAATGTCAGTACAGTACAATGTACACATGGAATTA |
| | | GGCCAGTAGTATCAACTCAACTGCTGTTAAATGGAAGTCTAGCAGAAGAA |
| | | GAGGTAGTAATTAGATCTAGCAATTTCACGGACAATGCTAAAATCATAAT |
| | | AGTACAGCTGAATGAAACTGTAGAAATTAATTGTACAAGACCCAACAACA |
| | | ATACAAGAAAAGGGATAACTCTAGGACCAGGGAGAGTATTTTATACAACA |
| | | GGAAAAATAGTAGGAGATATAAGAAAAGCACATTGTAACATTAGTAAAGT |
| | | AAAATGGCATAACACTTTAAAAAGGGTAGTTAAAAAATTAAGAGAAAAAT |
| | | TTGAAAATAAAACAATAATCTTTAATAAATCCTCAGGGGGGGACCCAGAA |
| | | ATTGTAATGCACAGCTTTAATTGTGGAGGGGAATTTTTCTACTGTAATAC |
| | | AAAAAAACTGTTTAATAGTACTTGGAATGGTACTGAAGGGTCATATAACA |
| | | TTGAAGGAAATGACACTATCACACTCCCATGCAGAATAAAACAAATTATA |
| | | AACATGTGGCAGGAAGTAGGAAAAGCAATGTATGCCCCTCCCATCAGTGG |
| | | ACAAATTTGGTGCTCATCAAATATTACAGGGCTGCTACTAACAAGAGATG |
| | | GTGGTAAGAACAGCAGCACCGAAATCTTCAGACCTGGAGGAGGAGATATA |
| | | AGGGACAATTGGAGAAGTGAATTATATAAATATAAAGTAGTAAGAGTTGA |
| | | ACCATTAGGAATAGCACCCACCAAGGCAAAAAGAAGAGTGGTGCAGAGAG |
| | | AAAAAAGAGCAGTGGGAATAGGAGCTGTGTTCCTTGGGTTCTTGGGAGCA |
| | | GCAGGAAGCACTATGGGCGCAGCGTCAATAACGCTGACGGTACAGGCCAG |
| | | ACAATTATTGTCTGGTATAGTGCAACAGCAGAACAATTTGCTGAGGGCTA |
| | | TTGAAGCGCAACAGCATATGTTGCAACTCACAGTCTGGGGCATCAAGCAG |
| | | CTCCAGGCAAGAGTCCTGGCTGTGGAAAGATACCTACAGGATCAACAGCT |
| | | CCTGGGGATTTGGGGTTGCTCTGGAAAACTCATCTGCACCACTACTGTGC |
| | | CTTGGAATACTAGTTGGAGTAATAAATCTCTGGATACAATTTGGGGTAAC |
| | | ATGACCTGGATGCAGTGGGAAAAAGAAATTAACAATTACACAGGCTTAAT |
| | | ATACAACTTGATTGAGGAATCGCAGAACCAACAAGAAAAGAATGAACAAG |
| | | AATTATTGGCATTAGATAAATGGGCAAGTTTGTGGAATTGGTTTAACATA |
| | | TCAAACTGGCTGTGGTATATAAAAATATTCATAATGATAGTAGGAGGCTT |
| | | GATAGGTTTAAGAATAGTTTTCAGTGTACTTTCTATAGTGAATAGAGTTA |
| | | GGCAGGGATACTCACCATTATCGTTTCAGACCCGCTTCCCAGCCTCGAGG |
| | | GGACCCGACAGGCCCGAAGGAATCGAAGAAGAAGGTGGAGACAGAGACAG |
| | | AGACAGATCCAGTCCATTAGTGGATGGATTCTTAGCAATCATCTGGGTCG |
| | | ACCTGCGGAGCCTGTTCCTCTTCAGCTACCACCGCTTGAGAGACTTACTC |
| | | TTGATTGTAACGAGGATTGTGGAACTTCTGGGACGCAGGGGGTGGGAACT |
| | | CCTCAAATACTTGTGGAATCTCCTGCAGTATTGGAGTCAGGAACTAAAGA |
| | | ATAGTGCTGTTAGCTTGCTTAACGCCACAGCCATAGCAGTAGGTGAGGGA |
| | | ACAGATAGAATTATAGAAATATTACAAAGAGCTGGTAGAGCTATTCTCAA |
| | | CATACCTACGAGAATAAGACAGGGCTTAGAAAGGGCTTTGCTATAAGCTT |
| | | ATGGGTGGAGCTATTTCCATGAGGCGGTCCAGGCCGTCTGGAAATCTGTA |
| | | CGAGAGACTCTTGCGGGCGCGTGGGGAGACTTATGGAAAACTCTTAGGAG |
| | | AGGTAAAAGATGGATACTCGCAATCCCCAGGAGGATTAGACAAGGGCTTG |
| | | AGCTCACTCTCTTGTGAGGGACAAAAATACAATCAGGGACAGTATATGAA |
| | | TACTCCATGGAGAAACCCAGCTAAAGAGAGAGAAAAATTAGCATACAGAA |
| | | AACAAAATATGGATGATATAGATAAGGAAGATGATGACTTGGTAGGGGTA |
| | | TCAGTGAGGCCAAAAGTTCCCCTAAGAACAATGAGTTACAAATTGGCAAT |
| | | AGACATGTCTCATTTTATAAAGGAAAAGGGGGGACTGGAAGGGATTTATT |
| | | ACAGTGCAAGAAGACATAGAATCTTAGACATATACTTAGAAAAGGAAGAA |
| | | GGCATCATACCAGATTGGCAGGATTACACCTCAGGACCAGGAATTAGATA |
| | | CCCAAAGACATTTGGCTGGCTATGGAAATTAGTCCCTGTAAATGTATCAG |
| | | ATGAGGCACAGGAGGATGAGGAGCATTATTTAATGCATCCAGCTCAAACT |
| | | TCCCAGTGGGATGACCCTTGGGGAGAGGTTCTAGCATGGAAGTTTGATCC |
| | | AACTCTGGCCTACACTTATGAGGCATATGTTAGATACCCAGAAGAGTTTG |
| | | GAAGCAAGTCAGGCCTGTCAGAGGAAGAGGTTAAAAGAAGGCTAACCGCA |
| | | AGAGGCCTTCTTAACATGGCTGACAAGAAGGAAACTCGCTGAATTCGAGC |
| | | TATCTACAGGGGACTTTCCGCTGGGGACTTTCCAGGGAGGCGTGGCCTGG |
| | | GCGGGACCGGGGAGTGGCGAGCCCTCAGATGCTGCATATAAGCAGCCGCT |
| | | TTTGCCTGTACTGGGTCTCTCTAGTTAGACCAGATCTGAGCCTGGGAGCT |
| | | CTCTGGCTAGCTGAGAACCCACTGCTTAAGCCTCAATAAAGCTTGCCTTG |
| | | AGTGCTTTAAGTAGTGTGTGCCCGTCTGTTGTGTGACTCTGGTAACTAGA |
| | | GATCCCTCAGACCATTTTAGTCAGTGTGGAAAATCTCTAGCA |
| SEQ ID | clone 1.27 | TGGAAGGGATTTATTACAGTGCAAGAAGACATAGAATCTTAGACATATAC |
| NO: 3 | DNA | TTAGAAAAGGAAGAAGGCATCATACCAGATTGGCAGGATTACACCTCAGG |
| | | ACCAGGAATTAGATACCCAAAGACATTTGGCTGGCTATGGAAATTAGTCC |
| | | CTGTAAATGTATCAGATGAGGCACAGGAGGATGAGGAGCATTATTTAATG |
| | | CATCCAGCTCAAACTTCCCAGTGGGATGACCCTTGGGGAGAGGTTCTAGC |
| | | ATGGAAGTTTGATCCAACTCTGGCCTACACTTATGAGGCATATGTTAGAT |
| | | ACCCAGAAGAGTTTGGAAGCAAGTCAGGCCTGTCAGAGGAAGAGGTTAAA |
| | | AGAAGGCTAACCGCAAGAGGCCTTCTTAACATGGCTGACAAGAAGGAAAC |
| | | TCGCTGAATTCGAGCTATCTACAGGGGACTTTCCGCTGGGGACTTTCCAG |
| | | GGAGGCGTGGCCTGGGCGGGACCGGGGAGTGGCGAGCCCTCAGATGCTGC |
| | | ATATAAGCAGCCGCTTTTGCCTGTACTGGGTCTCTCTAGTTAGACCAGAT |
| | | CTGAGCCTGGGAGCTCTCTGGCTAGCTGAGAACCCACTGCTTAAGCCTCA |
| | | ATAAAGCTTGCCTTGAGTGCTTTAAGTAGTGTGTGCCCGTCTGTTGTGTG |
| | | ACTCTGGTAACTAGAGATCCCTCAGACCATTTTAGTCAGTGTGGAAAATC |
| | | TCTAGCAGTGGCGCCCGAACAGGGACCGGAAAGCGAAAGAGAAACCAGAG |
| | | AAGCTCTCTCGACGCAGGACTCGGCTTGCTGAAGCGCGCACGGCAAGCGG |
| | | CGAGGGGCAGCGACCGGTGAGTACGCTAAAAATTTTGACTAGCGGAGGCT |
| | | AGAAGGAGAGAGATGGGTGCGAGAGCGTCAGTATTAAGCGGGGGAAAATT |
| | | GGATGCATGGGAAAAAATTCGGTTACGGCCAGGAGGAAAGAAAAAATATA |
| | | GACTAAAACATCTAGTATGGGCAAGCAGGGAGCTAGAACGATTTGCACTT |
| | | AATCCTGGCCTTTTAGAGACATCAGATGGCTGTAAACAAATAATAGGACA |
| | | GCTACAACCAGCTATCCGGACAGGATCAGAAGAACTTAGATCATTATTTA |
| | | ATACAGTAGCAACCCTCTATTGTGTACATGAAAGGATAGAGGTAAAAGAC |
| | | ACCAAGGAAGCTTTAGAGAAGATAGAGGAAGAGCAAAACAAAAGTAAGAA |
| | | AAAAGCACAGCAAGCAGCAGCTGACACAGGACACAGCAATCAGGTCAGCC |
| | | AAAATTACCCTATAGTGCAGAACATCCAGGGGCAAATGGTACATCAGGCC |
| | | CTATCACCTAGAACTTTAAATGCGTGGGTAAAAGTAGTAGAAGAGAAGGC |
| | | TTTTAGCCCAGAAGTAATACCCATGTTTTCAGCATTATCAGAAGGAGCCA |
| | | CCCCACAAGATTTAAACACCATGCTAAACACAGTGGGGGGACATCAAGCA |
| | | GCCATGCAAATGTTAAAAGAGACCATCAATGAGGAAGCTGCAGAATGGGA |
| | | TAGAGTGCATCCAGTGCATGCAGGGCCTATTGCACCAGGCCAGATGAGAG |
| | | AGCCAAGGGGAAGTGACATAGCAGGAACTACTAGTACCCTTCAGGAACAA |
| | | ATAGGATGGATGACACATAATCCACCTATCCCAGTAGGAGAAATCTATAA |
| | | AAGATGGATAATCCTGGGATTAAATAAAATAGTAAGAATGTATAGCCCTA |
| | | CCAGCATTCTGGACATAAGACAAGGACCAAAGGAACCCTTTAGAGACTAT |
| | | GTAGACCGGTTCTATAAAACCCTAAGAGCCGAGCAAGCTACACAGGAGGT |
| | | AAAAAATTGGATGACAGAAACCTTGTTGGTCCAAAATGCGAACCCAGATT |
| | | GTAAAACTATTTTAAAAGCATTGGGACCAGCAGCCACACTAGAAGAAATG |
| | | ATGACAGCATGTCAGGGAGTGGGAGGACCCGGCCATAAAGCAAGAGTTTT |
| | | GGCTGAAGCAATGAGCCAAGTAACAAACTCAGCTACCATAATGATGCAGA |
| | | GAGGCAATTTTAGGAACCAAAGAAAAACTGTTAAGTGTTTCAATTGTGGC |
| | | AAAGAAGGGCACATAGCCAAAAATTGCAGGGCTCCTAGGAAAAAGGGCTG |
| | | TTGGAAATGTGGAAAGGAAGGACACCAAATGAAAGATTGTACTGAGAGAC |
| | | AGGCTAATTTTTTAGGGAAGATCTGGCCTTCCCACAAGGGAAGGCCAGGA |
| | | AATTTTCTTCAGAGCAGACCAGAGCCAACAGCCCCATCAGAAGAGAGCGT |
| | | CAAGTTTGGAGAAGAGACAACAACTCCCTCTCAGAAGCAGGAGCCGATAG |
| | | ACAAGGAACTGTATCCTTTAACTTCCCTCAGATCACTCTTTGGCAACGAC |
| | | CCCTCGTCACAATAAAGATAGGGGGGCAACTAAAGGAAGCTCTATTAGAT |
| | | ACAGGAGCAGATGATACAGTATTAGAAGACATGGATTTGCCAGGAAGATG |
| | | GAAACCAAAAATGATAGGGGGAATTGGAGGTTTTATCAAAGTAAGACAGT |
| | | ATGATCAGATACCCATAGATATCTGTGGACATAAAGCTGTAGGTACAGTA |
| | | TTAGTAGGACCTACACCTGTCAACATAATTGGAAGAAATCTGTTGACTCA |
| | | GATTGGTTGCACTTTAAATTTTCCCATTAGTCCTATTGAAACTGTACCAG |
| | | TAAAATTAAAGCCAGGAATGGATGGCCCAAAAGTCAAACAATGGCCATTG |
| | | ACAGAAGAAAAAATAAAAGCATTAGTAGAAATTTGTACAGAAATGGAAAA |
| | | GGAAGGAAAGATTTCAAAAATTGGGCCTGAAAATCCATACAATACTCCAG |
| | | TATTTGCCATAAAGAAAAAAGACAGTACTAAATGGAGAAAATTAGTAGAT |
| | | TTCAGAGAACTTAATAGGAAAACTCAAGACTTCTGGGAAGTTCAATTAGG |
| | | AATACCACATCCCGCAGGGTTAAAAAAGAAAAAATCAGTAACAGTACTGG |
| | | ATGTGGGTGATGCATATTTTTCAGTTCCCTTAGATAAAGACTTCAGGAAG |
| | | TATACTGCATTTACCATACCTAGTATAAACAATGAGACACCAGGGATTAG |
| | | ATATCAGTACAATGTGCTTCCACAGGGATGGAAAGGATCACCAGCAATAT |
| | | TCCAAAGTAGCATGACAAAAACCTTAGAGCCTTTTAGAAAACAAAATCCA |
| | | GACATAATTATCTATCAATACATGGATGATTTGTATGTAGGATCTGACTT |
| | | AGAAATAGGGCAGCATAGAACAAAAATAGAGGAACTGAGACAACATCTGT |
| | | TAAAGTGGGGATTTACCACACCAGACAAAAAACATCAGAAAGAACCTCCA |
| | | TTCCTTTGGATGGGTTATGAACTCCATCCTGATAAATGGACAGTACAGCC |
| | | TATAGTGCTGCCAGAAAAAGACAGCTGGACTGTCAATGACATACAGAAGT |
| | | TAGTGGGAAAATTAAATTGGGCAAGTCAAATTTATGCAGGGATTAAAGTA |
| | | AAGCAATTATGTAAACTCCTTAGGGGAACCAAAGCACTTACAGAAGTAAT |
| | | ACCACTAACAAAAGAAGCAGAGCTAGAACTGGCAGAAAACAGGGAGATTT |
| | | TAAAGGAACCAGTACATGGAGTGTATTATGACCCATCAAAAGACTTAATA |
| | | GTAGAAATACAGAAGCAGGGGCAAGGCCAATGGACATATCAAATTTTTCA |
| | | AGAGCCATTTAAAAATCTGAAAACAGGAAAATATGCAAAAACGAGGGGTG |
| | | CCCACACTAATGATGTAAAACAATTAACAGAGGCAGTGCAAAAAATAGCC |
| | | AATGAAAGCATAGTAATATGGGGAAAGATTCCTAAATTTAAATTACCCAT |
| | | ACAAAAAGAAACATGGGAAACATGGTGGACAGAGTATTGGCAAGCCACCT |
| | | GGATTCCTGAGTGGGAGTTTGTCAATACCCCTCCCTTAGTGAAATTATGG |
| | | TACCAGTTAGAAAAAGAACCCATAGTAGGAGCAGAAACTTTCTATGTAGA |
| | | TGGGGCAGCTAACAGGGAGACTAAATTAGGAAAAGCAGGATATGTTACTA |
| | | GCAGAGGAAGGCAAAAAGTTGTCTCCCTAACAGACACAACAAATCAGAAA |
| | | ACTGAGTTACAAGCAATTCACCTAGCTTTGCAGGATTCAGGATTAGAAGT |
| | | AAACATAGTAACAGACTCACAATATGCATTAGGAATCATTCAAGCACAAC |
| | | CAGATAAAAGTGAATCAGAGTTAGTCAGTCAAATAATAGAACAGCTAATA |
| | | AAAAAGGAAAAAGTCTACCTGGCATGGGTACCAGCACACAAAGGAATTGG |
| | | AGGAAATGAACAGGTAGATAAATTAGTCAGTGCTGGAATCAGGAGAGTAC |
| | | TATTTCTAGATGGAATAGAGAAGGCCCAAGAAGAACATGAGAAATATCAT |
| | | AATAATTGGAGAGCAATGGCTAGTGAATTTAACCTGCCAGCTGTAGTAGC |
| | | AAAAGAGATAGTAGCCTGCTGTGATAAGTGCCAGGTAAAAGGAGAAGCCA |
| | | TGCATGGACAAGTAGACTGCAGTCCAGGAATATGGCAACTAGATTGTACA |
| | | CATTTAGAAGGAAAAGTTATCCTGGTAGCAGTTCATGTAGCCAGTGGATA |
| | | TATAGAAGCAGAGGTTATTCCAGCAGAGACAGGACAGGAAACAGCATACT |
| | | TTATTTTAAAATTAGCAGGAAGATGGCCAGTAAAAACAATACATACAGAC |
| | | AATGGCAGTAATTTCACCAGTACTACGGTTAAGGCCGCCTGTTGGTGGGC |
| | | AGGGATCAAGCAGGAATCTGGCATTCCCTACAATCCCCAAAGTCAAGGAG |
| | | TAGTAGAATCTATGAATAAAGAATTAAAGAAAATTATAGAACAAGTAAGA |
| | | GATCAGGCTGAACATCTTAAGACAGCAGTACAAATGGCAGTATTCATTCA |
| | | CAATTTTAAAAGAAAAGGGGGGATTGGGGGGTACAGTGCAGGGGAAAGAA |
| | | TAGTAGACATAATAGCATCAGACATACAAACTAAAGAACTACAAAAACAA |
| | | ATTACAAAAATTCAAAATTTTCGGGTTTATTACAGGGACAGCAGAGATCC |
| | | ACTTTGGAAAGGACCAGCAAAGCTTCTTTGGAAAGGTGAAGGGGCAGTAG |
| | | TAATACAAGATAAGAGTGACATAAAAGTAGTGCCAAGAAGAAAAGCAAAG |
| | | ATTATCAGGGATTATGGAAAACAGATGGCAGGTGATGATTGTGTGGCAAG |
| | | TAGACAGGATGAGGATTAGAACATGGAAAAGTTTAGTAAAACACCATATG |
| | | TATGTTTCAAAGAAAGCTAAGGGATGGTTTTATAGACATCACTATGAAAG |
| | | CACTCATCCAAGAATAAGTTCAGAAGTACATATCCCACTAGGGGATGCTA |
| | | GCTTGGTAGTAACAACATATTGGGGTCTACATACAGGAGAAAGAGACTGG |
| | | CATTTGGGTCAGGGAGTCTCCATAGAATGGAGGAAAAGGAGATACAGCAC |
| | | ACAAGTAGACCCTGACCTAGCAGACCAACTAATTCATCTGTACTACTTTG |
| | | ATTGTTTTTCAGAATCTGCTATAAGAAATGCCATATTAGGACATAGAGTT |
| | | AGTCCTAGGTGTGAATATCAAGCAGGACATAACAAGGTAGGATCTCTACA |
| | | GTACTTGGCACTAGCAGCATTAGTAACACCAAGAAAGATAAAGCCACCTT |
| | | TGCCTAGTGTTGCGAAACTGACAGAGGACAGATGGAACAAGTCCCACAAG |
| | | ACCAAGGGCCACAGAGGGAGCCATACAATGAATGGACACTAAAGCTTTTA |
| | | GAGGAGCTTAAGAATGAAGCTGTCAGACATTTCCCTAGACCATGGCTTCA |
| | | TGGCCTAGGGCAATATATCTATGAAACTTATGAGGATACTTGGGCAGGAG |
| | | TGGAAGCCATAATAAGAATTCTGCAACAATTGCTGCTTATTCATTTCAGA |
| | | ATTGGGTGTCAACATAGCAGAATAGGCATTATTCGACAGAGGAGAACAAG |
| | | AAATGGAGCCAGTAGATCCTAGACTAGAGCCCTGGAAGCATCCAGGAAGT |
| | | CAGCCTAAGACTGCCTGTACCAATTGCTATTGCAAAAAGTGTTGCTTGCA |
| | | TTGCCAAGTTTGCTTCATAACAAAAGGCTTAGGCATCTCCTATGGCAGGA |
| | | AGAAGCGGAAAAAGCGACGAAGATCTCCTCAACACAGTCAGACTGATCAA |
| | | GCTTCTCTATCAAAGCAGTAAGTAGTACATGTAATGCAACCTTTAGTAAT |
| | | ATTAGCAATAGTAGCATTAGTAGTAGCACTAATAATAGTCATAGTTGTAT |
| | | GGTCCATTGTATTAATAGAATATAGAAAAATATTAAGACAAAAGAAAATA |
| | | GACAGGTTAATTGATAGAATAAGAGAAAAAGCAGAAGACAGTGGCAATGA |
| | | GAGTGATGGGGATCAGGAAGAATTATCAGCACTTGTGGAAAGGGGGCACC |
| | | TTGCTCCTTGGGATATTGATGATCTGTAGTGCTGCAGAACAATTGTGGGT |
| | | CACAGTCTATTATGGGGTACCTGTGTGGAAAGAAGCAAACACCACTCTAT |
| | | TTTGTGCATCAGATGCTAAGGCATATGATACAGAGGTACATAATGTTTGG |
| | | GCCACACATGCCTGTGTACCCACAGACCCCAACCCACAAGAAATACTATT |
| | | GGAAAATGTGACAGAAGATTTTAACATGTGGAAAAATAACATGGTAGAAC |
| | | AGATGCATGAGGATATAATCAGTTTATGGGATCAAAGTCTAAAGCCATGT |
| | | GTAAAATTAACCCCACTCTGTGTTACTTTACATTGCACTGATTTGAAGAA |
| | | TGGTACTAATTTGAAGAATGGTACTAAAATCATTGGGAAATCAATAAGAG |
| | | GAGAAATAAAAAACTGCTCTTTCAATGTCACCAAAAACATAATAGATAAG |
| | | GTGAAAAAAGAATATGCGCTTTTCTATAGACATGATGTAGTACCAATAGA |
| | | TAGGAATATTACTAGCTATAGGTTAATAAGTTGTAACACCTCAACCCTTA |
| | | CACAGGCCTGTCCAAAGGTATCCTTTGAGCCAATTCCCATACATTATTGT |
| | | GCCCCGGCTGGTTTTGCGATTCTAAAATGTAAAGATAAGAAGTTCAATGG |
| | | AACGGGACCATGTACAAATGTCAGTACAGTACAATGTACACACGGAATTA |
| | | GGCCAGTAGTATCAACTCAACTGCTGTTAAATGGAAGTCTAGCAGAAGAA |
| | | GAGGTAGTAATTAGATCTAGCAATTTCACGGACAATGCTAAAATCATAAT |
| | | AGTACAGCTGAATGAAACTGTAGAAATTAATTGTACAAGACCCAACAACA |
| | | ATACAAGAAAAGGGATAACTCTAGGACCAGGGAGAGTATTTTATACAACA |
| | | GGAAAAATAGTAGGAGATATAAGAAAAGCACATTGTAACATTAGTAAAGT |
| | | AAAATGGCATAACACTTTAAAAAGGGTAGTTAAAAAATTAAGAGAAAAAT |
| | | TTGAAAATAAAACAATAATCTTTAATAAATCCTCAGGGGGGGACCCAGAA |
| | | ATTGTAATGCACAGCTTTAATTGTGGAGGGGAATTTTTCTACTGTAATAC |
| | | AAAAAAACTGTTTAATAGTACTTGGAATGGTACTGAAGGGTCATATAACA |
| | | TTGAAGGAAATGACACTATCACACTCCCATGCAGAATAAAACAAATTATA |
| | | AACATGTGGCAGGAAGTAGGAAAAGCAATGTATGCCCCTCCCATCAGTGG |
| | | ACAAATTTGGTGCTCATCAAATATTACAGGGCTGCTACTAACAAGAGATG |
| | | GTGGTAAGAACAGCAGCACCGAAATCTTCAGACCTGGAGGAGGAGATATA |
| | | AGGGACAATTGGAGAAGTGAATTATATAAATATAAAGTAGTAAGAGTTGA |
| | | ACCATTAGGAATAGCACCCACCAAGGCAAAAAGAAGAGTGGTGCAGAGAG |
| | | AAAAAAGAGCAGTGGGAATAGGAGCTGTGTTCCTTGGGTTCTTGGGAGCA |
| | | GCAGGAAGCACTATGGGCGCAGCGTCAATAACGCTGACGGTACAGGCCAG |
| | | ACAATTATTGTCTGGTATAGTGCAACAGCAGAACAATTTGCTGAGGGCTA |
| | | TTGAAGCGCAACAGCATATGTTGCAACTCACAGTCTGGGGCATCAAGCAG |
| | | CTCCAGGCAAGAGTCCTGGCTGTGGAAAGATACCTACAGGATCAACAGCT |
| | | CCTGGGGATTTGGGGTTGCTCTGGAAAACTCATCTGCACCACTACTGTGC |
| | | CTTGGAATACTAGTTGGAGTAATAAATCTCTGGATACAATTTGGGGTAAC |
| | | ATGACCTGGATGCAGTGGGAAAAAGAAATTAACAATTACACAGGCTTAAT |
| | | ATACAACTTGATTGAAGAATCGCAGAACCAACAAGAAAAGAATGAACAAG |
| | | AATTATTGGCATTAGATAAATGGGCAAGTTTGTGGAATTGGTTTAACATA |
| | | TCAAACTGGCTGTGGTATATAAAAATATTCATAATGATAGTAGGAGGCTT |
| | | GATAGGTTTAAGAATAGTTTTCAGTGTACTTTCTATAGTGAATAGAGTTA |
| | | GGCAGGGATACTCACCATTATCGTTTCAGACCCGCTTCCCAGCCTCGAGG |
| | | GGACCCGACAGGCCCGAAGGAATCGAAGAAGAAGGTGGAGACAGAGACAG |
| | | AGACAGATCCAGTCCATTAGTGGATGGATTCTTAGCAATCATCTGGGTCG |
| | | ACCTGCGGAGCCTGTTCCTCTTCAGCTACCACCGCTTGAGAGACTTACTC |
| | | TTGATTGTAACGAGGATTGTGGAACTTCTGGGACGCAGGGGGTGGGAACT |
| | | CCTCAAATACTTGTGGAATCTCCTGCAGTATTGGAGTCAGGAACTAAAGA |
| | | ATAGTGCTGTTAGCTTGCTTAACGCCACAGCCATAGCAGTAGGTGAGGGA |
| | | ACAGATAGAATTATAGAAATATTACAAAGAGCTGGTAGAGCTATTCTCAA |
| | | CATACCTACGAGAATAAGACAGGGCTTAGAAAGGGCTTTGCTATAAGCTT |
| | | ATGGGTGGAGCTATTTCCATGAGGCGGTCCAGGCCGTCTGGAAATCTGTA |
| | | CGAGAGACTCTTGCGGGCGCGTGGGGAGACTTATGGAAAACTCTTAGGAG |
| | | AGGTAAAAGATGGATACTCGCAATCCCCAGGAGGATTAGACAAGGGCTTG |
| | | AGCTCACTCTCTTGTGGGGGACAAAAATACAATCAGGGACAGTATATGAA |
| | | TACTCCATGGAGAAACCCAGCTAAAGAGAGAGAAAAATTAGCATACAGAA |
| | | AACAAAATATGGATGATATAGATAAGGAAGATGATGACTTGGTAGGGGTA |
| | | TCAGTGACGCCAAAAGTTCCCCTAAGAACAATGAGTTACAAATTGGCAAT |
| | | AGACATGTCTCATTTTATAAAAGAAAAGGGGGGACTGGAAGGGATTTATT |
| | | ACAGTGCAAGAAGACATAGAATCTTAGACATATACTTAAAAAAGGAAGAA |
| | | GGCATCATACCAGATTGGCAGGATTACACCTCAGGACCAGGAATTAGATA |
| | | CCCAAAGACATTTGGCTGGCTATGGAAATTAGTCCCTGTAAATGTATCAG |
| | | ATGAGGCACAGGAGGATGAGGAGCATTATTTAATGCATCCAGCTCAAACT |
| | | TCCCAGTGGGATGACCCTTGGGGAGAGGTTCTAGCATGGAAGTTTGATCC |
| | | AACTCTGGCCTACACTTATGAGGCATATGTTAGATACCCAGAAGAGTTTG |
| | | GAAGCAAGTCAGGCCTGTCAGAGGAAGAGGTTAAAAGAAGGCTAACCGCA |
| | | AGAGGCCTTCTTAACATGGCTGACAAGAAGGAAACTCGCTGAATTCGAGC |
| | | TATCTACAGGGGACTTTCCGCTGGGGACTTTCCAGGGAGGCGTGGCCTGG |
| | | GCGGGACCGGGGAGTGGCGAGCCCTCAGATGCTGCATATAAGCAGCCGCT |
| | | TTTGCCTGTACTGGGTCTCTCTAGTTAGACCAGATCTGAGCCTGGGAGCT |
| | | CTCTGGCTAGCTGAGAACCCACTGCTTAAGCCTCAATAAAGCTTGCCTTG |
| | | AGTGCTTTAAGTAGTGTGTGCCCGTCTGTTGTGTGACTCTGGTAACTAGA |
| | | GATCCCTCAGACCATTTTAGTCAGTGTGGAAAATCTCTAGCA |
| SEQ ID | clone 1.10 | TGGAAGGGATTTATTACAGTGCAAGAAGACATAGAATCTTAGACATATAC |
| NO: 4 | DNA | TTAGAAAAGGAAGAAGGCATCATACCAGATTGGCAGGATTACACCTCAGG |
| | | ACCAGGAATTAGATACCCAAAGACATTTGGCTGGCTATGGAAATTAGTCC |
| | | CTGTAAATGTATCAGATGAGGCACAGGAGGATGAGGAGCATTATTTAATG |
| | | CATCCAGCTCAAACTTCCCAGTGGGATGACCCTTGGGGAGAGGTTCTAGC |
| | | ATGGAAGTTTGATCCAACTCTGGCCTACACTTATGAGGCATATGTTAGAT |
| | | ACCCAGAAGAGTTTGGAAACAAGTCAGGCCTGTCAGAGGAAGAGGTTAAA |
| | | AGAAGGCTAACCGCAAGAGGCCTTCTTAACATGGCTGACAAGAAGGAAAC |
| | | TCGCTGAATTCGAGCTATCTACAGGGGACTTTCCGCTGGGGACTTTCCAG |
| | | GGAGGCGTGGCCTGGGCGGGACCGGGGAGTGGCGAGCCCTCAGATGCTGC |
| | | ATATAAGCAGCCGCTTTTGCCTGTACTGGGTCTCTCTAGTTAGACCAGAT |
| | | CTGAGCCTGGGAGCTCTCTGGCTAGCTGAGAACCCACTGCTTAAGCCTCA |
| | | ATAAAGCTTGCCTTGAGTGCTTTAAGTAGTGTGTGCCCGTCTGTTGTGTG |
| | | ACTCTGGTAACTAGAGATCCCTCAGACCATTTTAGTCAGTGTGGAAAATC |
| | | TCTAGCAGTGGCGCCCGAACAGGGACTTGAAAGCGAAAGAGAAACCAGAG |
| | | AAGCTCTCTCGACGCAGGACTCGGCTTGCTGAAGCGCGCACGGCAAGCGG |
| | | CGAGGGGCAGCGACCGGTGAGTACGCTAAAAATTTTGACTAGCGGAGGCT |
| | | AGAAGGAGAGAGATGGGTGCGAGAGCGTCAGTATTAAGCGGGGGAAAATT |
| | | GGATGCATGGGAAAAAATTCGGTTACGGCCAGGAGGAAAGAAAAAATATA |
| | | GACTAAAACATCTAGTATGGGCAAGCAGGGAGCTAGAACGATTTGCACTT |
| | | AATCCTGGCCTTTTAGAGACATCAGATGGCTGTAAACAAATAATAGGACA |
| | | GCTACAACCAGCTATCCGGACAGGATCAGAAGAACTTAGATCATTATTTA |
| | | ATACAGTAGCAACCCTCTATTGTGTACATGAAAGGATAGAGGTAAAAGAC |
| | | ACCAAGGAAGCTTTAGAGAAGATAGAGGAAGAGCAAAACAAAAGTAAGAA |
| | | AAAAGCACAGCAAGCAGCAGCTGACACAGGACACAGCAATCAGGTCAGCC |
| | | AAAATTACCCTATAGTGCAGAACATCCAGGGGCAAATGGTACATCAGGCC |
| | | CTATCACCTAGAACTTTAAATGCGTGGGTAAAAGTAGTAGAAGAGAAGGC |
| | | TTTTAGCCCAGAAGTAATACCCATGTTTTCAGCATTATCAGAAGGAGCCA |
| | | CCCCACAAGATTTAAACACCATGCTAAACACAGTGGGGGGACATCAAGCA |
| | | GCCATGCAAATGTTAAAAGAGACCATCAATGAGGAAGCTGCAGAATGGGA |
| | | TAGAGTGCATCCAGTGCATGCAGGGCCTATTGCACCAGGCCAGATGAGAG |
| | | AACCAAGGGGAAGTGACATAGCAGGAACTACTAGTACCCTTCAGGAACAA |
| | | ATAGGATGGATGACACATAATCCACCTATCCCAGTAGGAGAAATCTATAA |
| | | AAGATGGATAATCCTGGGATTAAATAAAATAGTAAGAATGTATAGCCCTA |
| | | CCAGCATTCTGGACATAAGACAAGGACCAAAGGAACCCTTTAGAGACTAT |
| | | GTAGACCGGTTCTATAAAACCCTAAGAGCCGAGCAAGCTACACAGGAGGT |
| | | AAAAAATTGGATGACAGAAACCTTGTTGGTCCAAAATGCGAACCCAGATT |
| | | GTAAAACTATTTTAAAAGCATTGGGACCAGCAGCCACACTAGAAGAAATG |
| | | ATGACAGCATGTCAGGGAGTGGGAGGACCCGGCCATAAAGCAAGAGTTTT |
| | | GGCTGAAGCAATGAGCCAAGTAACAAATTCAGCTACCATAATGATGCAGA |
| | | GAGGCAATTTTAGGAACCAAAGAAAAACTGTTAAGTGTTTCAATTGTGGC |
| | | AAAGAAGGGCACATAGCCAAAAATTGCAGGGCTCCTAGGAAAAAGGGCTG |
| | | TTGGAAATGTGGAAAGGAAGGACACCAAATGAAAGATTGTACTGAGAGAC |
| | | AGGCTAATTTTTTAGGGAAGATCTGGCCTTCCCACAAGGGAAGGCCAGGA |
| | | AATTTTCTTCAGAGCAGACCAGAGCCAACAGCCCCATCAGAAGAGAGCGT |
| | | CAGGTTTGGAGAAGAGACAACAACTCCCTCTCAGAAGCAGGAGCCGATAG |
| | | ACAAGGAACTGTATCCTTTAACTTCCCTCAGATCACTCTTTGGCAACGAC |
| | | CCCTCGTCACAATAAAGATAGGGGGGCAACTAAAGGAAGCTCTATTAGAT |
| | | ACAGGAGCAGATGATACAGTATTAGAAGACATGGATTTGCCAGGAAGATG |
| | | GAAACCAAAAATGATAGGGGGAATTGGAGGTTTTATCAAAGTAAGACAGT |
| | | ATGATCAGATACCCATAGATATCTGTGGACATAAAGCTGTAGGTACAGTA |
| | | TTAGTAGGACCTACACCTGTCAACATAATTGGAAGAAATCTGTTGACTCA |
| | | GATTGGTTGCACTTTAAATTTTCCCATTAGTCCTATTGAAACTGTACCAG |
| | | TAAAATTAAAGCCAGGAATGGATGGCCCAAAAGTCAAACAATGGCCATTG |
| | | ACAGAAGAAAAAATAAAAGCATTAGTAGAAATTTGTACAGAAATGGAAAA |
| | | GGAAGGAAAGATTTCAAAAATTGGGCCTGAAAATCCATACAATACTCCAG |
| | | TATTTGCCATAAAGAAAAAAGACAGTACTAAATGGAGAAAATTAGTAGAT |
| | | TTCAGAGAACTTAATAGGAAAACTCAAGACTTCTGGGAAGTTCAATTAGG |
| | | AATACCACATCCCGCAGGGTTAAAAAAGAAAAAATCAGTAACAGTACTGG |
| | | ATGTGGGTGATGCATATTTTTCAGTTCCCTTAGATAAAGACTTCAGGAAG |
| | | TATACTGCATTTACCATACCTAGTATAAACAATGAGACACCAGGGATTAG |
| | | ATATCAGTACAATGTGCTTCCACAGGGATGGAAAGGATCACCAGCAATAT |
| | | TCCAAAGTAGCATGACAAAAACCTTAGAGCCTTTTAGAAAACAAAATCCA |
| | | GACATAATTATCTATCAATACATGGATGATTTGTATGTAGGATCTGACTT |
| | | AGAAATAGGGCAGCATAGAACAAAAATAGAGGAACTGAGACAACATCTGT |
| | | TAAAGTGGGGATTTACCACACCAGACAAAAAACATCAGAAAGAACCTCCA |
| | | TTCCTTTGGATGGGTTATGAACTCCATCCTGATAAATGGACAGTACAGCC |
| | | TATAGTGCTGCCAGAAAAAGACAGCTGGACTGTCAATGACATACAGAAGT |
| | | TAGTGGGAAAATTAAATTGGGCAAGTCAAATTTATGCAGGGATTAAAGTA |
| | | AAGCAATTATGTAAACTCCTTAGGGGAACCAAAGCACTTACAGAAGTAAT |
| | | ACCACTAACAAAAGAAGCAGAGCTAGAACTGGCAGAAAACAGGGAGATTT |
| | | TAAAGGAACCAGTACATGGAGTGTATTATGACCCATCAAAAGACTTAATA |
| | | GTAGAAATACAGAAGCAGGGGCAAGGCCAATGGACATATCAAATTTTTCA |
| | | AGAGCCATTTAAAAATCTGAAAACAGGAAAATATGCAAAAACGAGGAGTG |
| | | CCCACACTAATGATGTAAAACAATTAACAGAGGCAGTGCAAAAAATAGCC |
| | | AATGAAAGCATAGTAATATGGGGAAAGATTCCTAAATTTAAATTACCCAT |
| | | ACAAAAAGAAACATGGGAAACATGGTGGACAGAGTATTGGCAAGCCACCT |
| | | GGATTCCTGAGTGGGAGTTTGTCAATACCCCTCCCTTAGTGAAATTATGG |
| | | TACCAGTTAGAAAAAGAACCCATAGTAGGAGCAGAAACTTTCTATGTAGA |
| | | TGGGGCAGCTAACAGGGAGACTAAATTAGGAAAAGCAGGATATGTTACTA |
| | | GCAGAGGAAGGCAAAAAGTTGTCTCCCTAACAGACACAACAAATCAGAAA |
| | | ACTGAGTTACAAGCAATTCACCTAGCTTTGCAGGATTCAGGATTAGAAGT |
| | | AAACATAGTAACAGACTCACAATATGCATTAGGAATCATTCAAGCACAAC |
| | | CAGATAAAAGTGAATCAGAGTTAGTCAGTCAAATAATAGAACAGCTAATA |
| | | AAAAAGGAAAAAGTCTACCTGGCATGGGTACCAGCACACAAAGGAATTGG |
| | | AGGAAATGAACAGGTAGATAAATTAGTCAGTGCTGGAATCAGGAGAGTAC |
| | | TATTTCTAGATGGAATAGAGAAGGCCCAAGAAGAACATGAGAAATATCAT |
| | | AATAATTGGAGAGCAATGGCTAGTGAATTTAACCTGCCAGCTGTAGTAGC |
| | | AAAAGAGATAGTAGCCTGCTGTGATAAGTGCCAGGTAAAAGGAGAAGCCA |
| | | TGCATGGACAAGTAGACTGCAGTCCAGGAATATGGCAACTAGATTGTACA |
| | | CATTTAGAAGGAAAAGTTATCCTGGTAGCAGTTCATGTAGCCAGTGGATA |
| | | TATAGAAGCAGAGGTTATTCCAGCAGAGACAGGACAGGAAACAGCATACT |
| | | TTATTTTAAAATTAGCAGGAAGATGGCCAGTAAAAACAATACATACAGAC |
| | | AATGGCAGTAATTTCACCAGTACTACGGTTAAGGCCGCCTGTTGGTGGGC |
| | | AGGGATCAAGCAGGAATTTGGCATTCCCTACAATCCCCAAAGTCAAGGAG |
| | | TAGTAGAATCTATGAATAAAGAATTAAAGAGAATTATAGAACAAGTAAGA |
| | | GATCAGGCTGAACATCTTAAGACAGCAGTACAAATGGCAGTATTCATTCA |
| | | CAATTTTAAAAGAAAAGGGGGGATTGGGGGGTACAGTGCAGGGGAAAGAA |
| | | TAGTAGACATAATAGCATCAGACATACAAACTAAAGAACTACAAAAACAA |
| | | ATTACAAAAATTCAAAATTTTCGGGTTTATTACAGGGACAGCAGAGATCC |
| | | ACTTTGGAAAGGACCAGCAAAGCTTCTTTGGAAAGGTGAAGGGGCAGTAG |
| | | TAATACAAGATAAGAGTGACATAAAAGTAGTGCCAAGAAGAAAAGCAAAG |
| | | ATTATCAGGGATTATGGAAAACAGATGGCAGGTGATGATTGTGTGGCAAG |
| | | TAGACAGGATGAGGATTAGAACATGGAAAAGTTTAGTAAAACACCATATG |
| | | TATGTTTCAAAGAAAGCTAAGGGATGGTTTTATAGACATCACTATGAAAG |
| | | CACTCATCCAAGAATAAGTTCAGAAGTACATATCCCACTAGGGGATGCTA |
| | | GCTTGGTAGTAACAACATATTGGGGTCTACATACAGGAGAAAGAGACTGG |
| | | CATTTGGGTCAGGGAGTCTCCATAGAATGGAGGAAAAGGAGATACAGCAC |
| | | ACAAGTAGACCCTGACCTAGCAGACCAACTAATTCATCTGTACTACTTTG |
| | | ATTGTTTTTCAGAATCTGCTATAAGAAATGCCATATTAGGACATAGAGTT |
| | | AGTCCTAGGTGTGAATATCAAGCAGGACATAACAAGGTAGGATCTCTACA |
| | | GTACTTGGCACTAGCAGCATTAGTAACACCAAGAAAGATAAAGCCACCTT |
| | | TGCCTAGTGTTGCGAAACTGACAGAGGACAGATGGAACAAGTCCCACAAG |
| | | ACCAAGGGCCACAGAGGGAGCCATACAATGAATGGACACTAAAGCTTTTA |
| | | GAGGAGCTTAAGAATGAAGCTGTCAGACATTTCCCTAGACCATGGCTTCA |
| | | TGGCCTAGGGCAATATATCTATGAAACTTATGAGGATACTTGGGCAGGAG |
| | | TGGAAGCCATAATAAGAATTCTGCAACAATTGCTGCTTATTCATTTCAGA |
| | | ATTGGGTGTCAACATAGCAGAATAGGCATTATTCGACAGAGGAGAACAAG |
| | | AAATGGAGCCAGTAGATCCTAGACTAGAGCCCTGGAAGCATCCAGGAAGT |
| | | CAGCCTAAGACTGCCTGTACCAATTGCTATTGCAAAAAGTGTTGCTTGCA |
| | | TTGCCAAGTTTGCTTCATAACAAAAGGCTTAGGCATCTCCTATGGCAGGA |
| | | AGAAGCGGAAAAAGCGACGAAGATCTCCTCAACACAGTCAGACTGATCAG |
| | | GCTTCTCTATCAAAGCAGTAAGTAGTACATGTAATGCAACCTTTAGTAAT |
| | | ATTAGCAATAGTAGCATTAGTAGTAGCACTAATAATAGTCATAGTTGTAT |
| | | GGTCCATTGTATTAATAGAATATAGAAAAATATTAAGACAAAAGAAAATA |
| | | GACAGGTTAATTGATAGAATAAGAGAAAAAGCAGAAGACAGTGGCAATGA |
| | | GAGTGATGGGGATCAGGAAGAATTATCAGCACTTGTGGAAAGGGGGCACC |
| | | TTGCTCCTTGGGATATTGATGATCTGTAGTGCTGCAGAACAATTGTGGGT |
| | | CACAGTCTATTATGGGGTACCTGTGTGGAAAGAAGCAAACACCACTCTAT |
| | | TTTGTGCATCAGATGCTAAGGCATATGATACAGAGGTACATAATGTTTGG |
| | | GCCACACATGCCTGTGTACCCACAGACCCCAACCCACAAGAAATACTATT |
| | | GGAAAATGTGACAGAAGATTTTAACATGTGGAAAAATAACATGGTAGAAC |
| | | AGATGCATGAGGATATAATCAGTTTATGGGATCAAAGTCTAAAGCCATGT |
| | | GTAAAATTAACCCCACTCTGTGTTACTTTACATTGCACTGATTTGAAGAA |
| | | TGGTACTAATTTGAAGAATGGTACTAATTTGAAGAATGGTACTAAAATCA |
| | | TTGGGAAATCAATAAGAGGAGAAATAAAAAACTGCTCTTTCAATGTCACC |
| | | AAAAACATAATAGATAAGGTGAAAAAAGAATATGCGCTTTTCTATAGACA |
| | | TGATGTAGTACCAATAGATAGGAATATTACTAGCTATAGGTTAATAAGTT |
| | | GTAACACCTCAACCCTTACACAGGCCTGTCCAAAGGTATCCTTTGAGCCA |
| | | ATTCCCATACATTATTGTGCCCCGGCTGGTTTTGCGATTCTAAAATGTAA |
| | | AGATAAGAAGTTCAATGGAACGGGACCATGTACAAATGTCAGTACAGTAC |
| | | AATGTACACATGGAATTAGGCCAGTAGTATCAACTCAACTGCTGTTAAAT |
| | | GGAAGTCTAGCAGAGGAAGAGGTAGTAATTAGATCTAGCAATTTCACGGA |
| | | CAATGCTAAAATCATAATAGTACAGCTGAATGAAACTGTAGAAATTAATT |
| | | GTACAAGACCCAACAACAATACAAGAAAAGGGATAACTCTAGGACCAGGG |
| | | AGAGTATTTTATACAACAGGAAAAATAGTAGGAGATATAAGAAAAGCACA |
| | | TTGTAACATTAGTAAAGTAAAATGGCATAACACTTTAAAAAGGGTAGTTA |
| | | AAAAATTAAGAGAAAAATTTGAAAATAAAACAATAATCTTTAATAAATCC |
| | | TCAGGGGGGGACCCAGAAATTGTAATGCACAGCTTTAATTGTGGAGGGGA |
| | | ATTTTTCTACTGTAATACAAAAAAACTGTTTAATAGTACTTGGAATGGTA |
| | | CTGAAGGGTCATATAACATTGAAGGAAATGACACTATCACACTCCCATGC |
| | | AGAATAAAACAAATTATAAACATGTGGCAGGAAGTAGGAAAAGCAATGTA |
| | | TGCCCCTCCCATCAGTGGACAAATTTGGTGCTCATCAAATATTACAGGGC |
| | | TGCTACTAACAAGAGATGGTGGTAAGAACAGCAGCACCGAAATCTTCAGA |
| | | CCTGGAGGAGGAGATATAAGGGACAATTGGAGAAGTGAATTATATAAATA |
| | | TAAAGTAGTAAGAGTTGAACCATTAGGAATAGCACCCACCAAGGCAAAAA |
| | | GAAGAGTGGTGCAGAGAGAAAAAAGAGCAGTGGGAATAGGAGCTGTGTTC |
| | | CTTGGGTTCTTGGGAGCAGCAGGAAGCACTATGGGCGCAGCGTCAATAAC |
| | | GCTGACGGTACAGGCCAGACAATTATTGTCCGGTATAGTGCAACAGCAGA |
| | | ACAATTTGCTGAGGGCTATTGAAGCGCAACAGCATATGTTGCAACTCACA |
| | | GTCTGGGGCATCAAGCAGCTCCAGGCAAGAGTCCTGGCTGTGGAAAGATA |
| | | CCTACAGGATCAACAGCTCCTGGGGATTTGGGGTTGCTCTGGAAAACTCA |
| | | TCTGCACCACTACTGTGCCTTGGAATACTAGTTGGAGTAATAAATCTCTG |
| | | GATACAATTTGGGGTAACATGACCTGGATGCAGTGGGAAAAAGAAATTAA |
| | | CAATTACACAGGCTTAATATACAACTTGATTGAAGAATCGCAGAACCAAC |
| | | AAGAAAAGAATGAACAAGAATTATTGGCATTAGATAAATGGGCAAGTTTG |
| | | TGGAATTGGTTTAACATATCAAACTGGCTGTGGTATATAAAAATATTCAT |
| | | AATGATAGTAGGAGGCTTGATAGGTTTAAGAATAGTTTTCAGTGTACTTT |
| | | CTATAGTGAATAGAGTTAGGCAGGGATACTCACCATTATCGTTTCAGACC |
| | | CGCTTCCCAGCCTCGAGGGGACCCGACAGGCCCGAAGGAATCGAAGAAGA |
| | | AGGTGGAGACAGAGACAGAGACAGATCCAGTCCATTAGTGGATGGATTCT |
| | | TAGCAATCATCTGGGTCGACCTGCGGAGCCTGTTCCTCTTCAGCTACCAC |
| | | CGCTTGAGAGACTTACTCTTGATTGTAACGAGGATTGTGGAACTTCTGGG |
| | | ACGCAGGGGGTGGGAACTCCTCAAATACTTGTGGAACCTCCTGCAGTATT |
| | | GGGGTCAGGAACTAAAGAATAGTGCTGTTAGCTTGCTTAACGCCACAGCC |
| | | ATAGCAGTAGGTGAGGGAACAGATAGAATTATAGAAATATTACAAAGAGC |
| | | TGGTAGAGCTATTCTCAACATACCTACGAGAATAAGACAGGGCTTAGAAA |
| | | GGGCTTTGCTATAAGCTTATGGGTGGAGCTATTTCCATGAGGCGGTCCAG |
| | | GCCGTCTGGAAATCTGTACGAGAGACTCTTGCGGGCGCGTGGGGAGACTT |
| | | ATGGAAAACTCTTAGGAGAGGTAAAAGATGGATACTTGCAATCCCCAGGA |
| | | GGATTAGACAAGGGCTTGAGCTCACTCTCTTGTGAGGGACAAAAATACAA |
| | | TCAGGGACAGTATATGAATACTCCATGGAGAAACCCAGCTAAAGAGAGAG |
| | | AAAAATTAGCATACAGAAAACAAAATATGGATGATATAGATAAGGAGGAT |
| | | GATGACTTGGTAGGGGTATCAGTGAGGCCAAAAGTTCCCCTAAGAACAAT |
| | | GAGTTACAAAGTGGCAATAGACATGTCTCATTTTATAAAAGAAAAGGGGG |
| | | GACTGGAAGGGATTTATTACAGTGCAAGAAGACATAGAATCTTAGACATA |
| | | TACTTAGAAAAGGAAGAAGGCATCATACCAGATTGGCAGGATTACACCTC |
| | | AGGACCAGGAATTAGATACCCAAAGACATTTGGCTGGCTATGGAAATTAG |
| | | TCCCTGTAAATGTATCAGATGAGGCACAGGAGGATGAGGAGCATTATTTA |
| | | ATGCATCCAGCTCAAACTTCCCAGTGGGATGACCCTTGGGGAGAGGTTCT |
| | | AGCATGGAAGTTTGATCCAACTCTGGCCTACACTTATGAGGCGTATGTTA |
| | | GATACCCAGAAGAGTTTGGAAGCAAGTCAGGCCTGTCAGAGGAAGAGGTT |
| | | AAAAGAAGGCTAACCGCAAGAGGCCTTCTTAACATGGCTGACAAGAAGGA |
| | | AACTCGCTGAATTCGAGCTATCTACAGGGGACTTTCCGCTGGGGACTTTC |
| | | CAGGGAGGCGTGGCCTGGGCGGGACCGGGGAGTGGCGAGCCCTCAGATGC |
| | | TGCATATAAGCAGCCGCTTTTGCCTGTACTGGGTCTCTCTAGTTAGACCA |
| | | GATCTGAGCCTGGGAGCTCTCTGGCTAGCTGAGAACCCACTGCTTAAGCC |
| | | TCAATAAAGCTTGCCTTGAGTGCTTTAAATAGTGTGTGCCCGTCTGTTGT |
| | | GTGACTCTGGTAACTAGAGATCCCTCAGACCATTTTAGTCAGTGTGGAAA |
| | | ATCTCTAGCA |
| SEQ ID | clone | TGGAAGGGATTTATTACAGTGCAAGAAGACATAGAATCTTAGACATATAC |
| NO: 5 | P10.21 DNA | TTAGAAAAGGAAGAAGGCATCATACCAGATTGGCAGGATTACACCTCAGG |
| | | ACCAGGAATTAGATACCCAAAGACATTTGGCTGGCTATGGAGATTAGTCC |
| | | CTGTAAATGTATCAGATGAGGCACAGGAGGATGAGGAGCATTATTTAATG |
| | | CATCCAGCTCAAACTTCCCAGTGGGATGACCCTTGGGGAGAGGTTCTAGC |
| | | ATGGAAGTTTGATCCAACTCTGGCCTACACTTATGAGGCATATGTTAGAT |
| | | ACCCAGAAGAGTTTGGAAGCAAGTCAGGCCTGTCAGAGGAAGAGGTTAAA |
| | | AGAAGGCTAACCGCAAGAGGCCTTCTTAACATGGCTGACAAGAAGGAAAC |
| | | TCGCTGAATTCGAGCTATCTACAGGGGACTTTCCGCTGGGGACTTTCCAG |
| | | GGAGGCGTGGCCTGGGCGGGACCGGGGAGTGGCGAGCCCTCAGATGCTGC |
| | | ATATAAGCAGCCGCTTTTGTCTGTACTGGGTCTCTCTAGTTAGACCAGAT |
| | | CTGAGCCTGGGAGCTCTCTGGCTAGCTGAGAACCCACTGCTTAAGCCTCA |
| | | ATAAAGCTTGCCTTGAGTGCTTTAAGTAGTGTGTGCCCGTCTGTTGTGTG |
| | | ACTCTGGTAACTAGAGATCCCTCAGACCATTTTAGTCAGTGTGGAAAATC |
| | | TCTAGCAGTGGCGCCCGAACAGGGACCGGAAAGCGAAAGAGAAACCAGAG |
| | | AAGCTCTCTCGACGCAGGACTCGGCTTGCTGAAGCGCGCACGGCAAGCGG |
| | | CGAGGGGCAGCGACCGGTGAGTACGCTAAAAATTTTGACTAGCGGAGGCT |
| | | AGAAGGAGAGAGATGGGTGCGAGAGCGTCAGTATTAAGCGGGGGAAAATT |
| | | GGATGCATGGGAAAAAATTCGGTTACGGCCAGGAGGAAAGAAAAAATATA |
| | | GACTAAAACATCTAGTATGGGCAAGCAGGGAGCTAGAACGATTTGCACTT |
| | | AATCCTGGCCTTTTAGAGACATCAGATGGCTGTAAACAAATAATAGGACA |
| | | GCTACAACCAGCTATCCGGACAGGATCAGAAGAATTTAGATCATTATTTA |
| | | ATACAGTAGCAACCCTCTATTGTGTACATGAAAGGATAGAGGTAAAAGAC |
| | | ACCAAGGAAGCTTTAGAGAAGATAGAGGAAGAGCAAAACAAAAGTAAGAA |
| | | AAAAGCACAGCAAGCAGCAGCTGACACAGGACACAGCAATCAGGTCAGCC |
| | | AAAATTACCCTATAGTGCAGAACATCCAGGGGCAAATGGTACATCAGGCC |
| | | CTATCACCTAGAACTTTAAATGCGTGGGTAAAAGTAGTAGAAGAGAAGGC |
| | | TTTTAGCCCAGAAGTAATACCCATGTTTTCAGCATTATCAGAAGGAGCCA |
| | | CCCCACAAGATTTAAACACCATGCTAAACACAGTGGGGGGACATCAAGCA |
| | | GCCATGCAAATGTTAAAAGAGACCATCAATGAGGAAGCTGCAGAATGGGA |
| | | TAGAGTGCATCCAGTGCATGCAGGGCCTATTGCACCAGGCCAGATGAGAG |
| | | AACCAAGGGGAAGTGACATAGCAGGAACTACTAGTACCCTTCAGGAACAA |
| | | ATAGGATGGATGACACATAATCCACCTATCCCAGTAGGAGAAATCTATAA |
| | | AAGATGGATAATCCTGGGATTAAATAAAATAGTAAGAATGTATAGCCCTA |
| | | CCAGCATTCTGGACATAAGACAAGGACCAAAGGAACCCTTTAGAGACTAT |
| | | GTAGACCGGTTCTATAAAACCCTAAGAGCCGAGCAAGCTACACAGGAGGT |
| | | AAAAAATTGGATGACAGAAACCTTGTTGGTCCAAAATGCGAACCCAGATT |
| | | GTAAAACTATTTTAAAAGCATTGGGACCAGCAGCCACACTAGAAGAAATG |
| | | ATGACAGCATGTCAGGGAGTGGGAGGACCCGGCCATAAAGCAAGAGTTTT |
| | | GGCTGAAGCAATGAGCCAAGTAACAAATTCAGCTACCATAATGATGCAGA |
| | | GAGGCAATTTTAGGAACCAAAGAAAAACTGTTAAGTGTTTCAATTGTGGC |
| | | AAAGAAGGGCACATAGCCAAAAATTGCAGGGCTTCTAGGAAAAAGGGCTG |
| | | TTGGAAATGTGGAAAGGAAGGACACCAAATGAAAGATTGTACTGAGAGAC |
| | | AGGCTAATTTTTTAGGGAAGATCTGGCCTTCCCACAAGGGAAGGCCAGGA |
| | | AATTTTCTTCAGAGCAGACCAGAGCCAACAGCCCCATCAGAAGAGAGCGT |
| | | CAAGTTTGGAGAAGAGACAACAACTCCCTCTCAGAAGCAGGAGCCGATAG |
| | | ACAAGGAACTGTATCCTTTAACTTCCCTCAGATCACTCTTTGGCAACGAC |
| | | CCCTCGTCACAATAAAGATAGGGGGGCAACTAAAGGAAGCTCTATTAGAT |
| | | ACAGGAGCAGATGATACAGTATTAGAAGACATGGATTTGCCAGGAAGATG |
| | | GAAACCAAAAATGATAGGGGGAATTGGAGGTTTTATCAAAGTAAGACAGT |
| | | ATGATCAGATACCCATAGATATCTGTGGACATAAAGCTGTAGGTACAGTA |
| | | TTAGTAGGACCTACACCTGTCAACATAATTGGAAGAAATCTGTTGACTCA |
| | | GATTGGTTGCACTTTAAATTTTCCCATTAGTCCTATTGAAACTGTACCAG |
| | | TAAAATTAAAGCCAGGAATGGATGGCCCAAAAGTCAAACAATGGCCATTG |
| | | ACAGAAGAAAAAATAAAAGCATTAGTAGAAATTTGTACAGAAATGGAAAA |
| | | GGAAGGAAAGATTTCAAAAATTGGGCCTGAAAATCCATACAATACTCCAG |
| | | TATTTGCCATAAAGAAAAAAGACAGTACTAAATGGAGAAAATTAGTAGAT |
| | | TTCAGAGAACTTAATAGGAAAACTCAAGACTTCTGGGAAGTTCAATTAGG |
| | | AATACCACATCCCGCAGGGTTAAAAAAGAAAAAATCAGTAACAGTACTGG |
| | | ATGTGGGTGATGCATATTTTTCAGTTCCCTTAGATAAAGACTTCAGGAAG |
| | | TATACTGCATTTACCATACCTAGTATAAACAATGAGACACCAGGGATTAG |
| | | ATATCAGTACAATGTGCTTCCACAGGGATGGAAAGGATCACCAGCAATAT |
| | | TCCAAAGTAGCATGACAAAAACCTTAGAGCCTTTTAGAAAACAAAATCCA |
| | | GACATAATTATCTATCAATACATGGATGATTTGTATGTAGGATCTGACTT |
| | | AGAAATAGGGCAGCATAGAACAAAAATAGAGGAACTGAGACAACATCTGT |
| | | TAAAGTGGGGATTTACCACACCAGACAAAAAACATCAGAAAGAACCTCCA |
| | | TTCCTTTGGATGGGTTATGAACTCCATCCTGATAAATGGACAGTACAGCC |
| | | TATAGTGCTGCCAGAAAAAGACAGCTGGACTGTCAATGACATACAGAAGT |
| | | TAGTGGGAAAATTAAATTGGGCAAGTCAAATTTATGCAGGGATTAAAGTA |
| | | AAGCAATTATGTAAACTCCTTAGGGGAACCAAAGCACTTACAGAAGTAAT |
| | | ACCACTAACAAAAGAAGCAGAGCTAGAACTGGCAGAAAACAGGGAGATTT |
| | | TAAAGGAACCAGTACATGGAGTGTATTATGACCCATCAAAAGACTTAATA |
| | | GTAGAAATACAGAAGCAGGGGCAAGGCCAATGGACATATCAAATTTTTCA |
| | | AGAGCCATTTAAAAATCTGAAAACAGGAAAATATGCAAAAACGAGGGGTG |
| | | CCCACACTAATGATGTAAAACAATTAACAGAGGCAGTGCAAAAAATAGCC |
| | | AATGAAAGCATAGTAATATGGGGAAAGATTCCTAAATTTAAATTACCCAT |
| | | ACAAAAAGAAACATGGGAAACATGGTGGACAGAGTATTGGCAAGCCACCT |
| | | GGATTCCTGAGTGGGAGTTTGTCAATACCCCTCCCTTAGTGAAATTATGG |
| | | TACCAGTTAGAAAAAGAACCCATAGTAGGAGCAGAAACTTTCTATGTAGA |
| | | TGGGGCAGCTAACAGGGAGACTAAATTAGGAAAAGCAGGATATGTTACTA |
| | | GCAGAGGAAGGCAAAAAGTTGTCTCCCTAACAGACACAACAAATCAGAAA |
| | | ACTGAGTTACAAGCAATTCACCTAGCTTTGCAGGATTCAGGATTAGAAGT |
| | | AAACATAGTAACAGACTCACAATATGCATTAGGAATCATTCAAGCACAAC |
| | | CAGATAAAAGTGAATCAGAGTTAGTCAGTCAAATAATAGAACAGCTAATA |
| | | AAAAAGGAAAAAGTCTACCTGGCATGGGTACCAGCACACAAAGGAATTGG |
| | | AGGAAATGAACAGGTAGATAAATTAGTCAGTGCTGGAATCAGGAGAGTAC |
| | | TATTTCTAGATGGAATAGAGAAGGCCCAAGAAGAACATGAGAAATATCAT |
| | | AATAATTGGAGAGCAATGGCTAGTGAATTTAACCTGCCAGCTGTAGTAGC |
| | | AAAAGAGATAGTAGCCTGCTGTGATAAGTGCCAGGTAAAAGGAGAAGCCA |
| | | TGCATGGACAAGTAGACTGCAGTCCAGGAATATGGCAACTAGATTGTACA |
| | | CATTTAGAAGGAAAAGTTATCCTGGTAGCAGTTCATGTAGCCAGTGGATA |
| | | TATAGAAGCAGAGGTTATTCCAGCAGAGACAGGACAGGAAACAGCATACT |
| | | TTATTTTAAAATTAGCAGGAAGATGGCCAGTAAAAACAATACATACAGAC |
| | | AATGGCAGTAATTTCACCAGTACTACGGTTAAGGCCGCCTGTTGGTGGGC |
| | | AGGGATCAAGCAGGAATTTGGCATTCCCTACAATCCCCAAAGTCAAGGAG |
| | | TAGTAGAATCTATGAATAAAGAATTAAAGAAAATTATAGAACAAGTAAGA |
| | | GATCAGGCTGAACATCTTAAGACAGCAGTACAAATGGCAGTATTCATTCA |
| | | CAATTTTAAAAGAAAAGGGGGGATTGGGGGGTACAGTGCAGGGGAAAGAA |
| | | TAGTAGACATAATAGCATCAGACATACAAACTAAAGAACTACAAAAACAA |
| | | ATTACAAAAATTCAAAATTTTCGGGTTTATTACAGGGACAGCAGAGATCC |
| | | ACTTTGGAAAGGACCAGCAAAGCTTCTTTGGAAAGGTGAAGGGGCAGTAG |
| | | TAATACAAGATAAGAGTGACATAAAAGTAGTGCCAAGAAGAAAAGCAAAG |
| | | ATTATCAGGGATTATGGAAAACAGATGGCAGGTGATGATTGTGTGGCAAG |
| | | TAGACAGGATGAGGATTAGAACATGGAAAAGTTTAGTAAAACACCATATG |
| | | TATGTTTCAAAGAAAGCTAAGGGATGGTTTTATAGACATCACTATGAAAG |
| | | CACTCATCCAAGAATAAGTTCAGAAGTACATATCCCACTAGGGGATGCTA |
| | | GCTTGGTAGTAACAACATATTGGGGTCTACATACAGGAGAAAGAGACTGG |
| | | CATTTGGGTCAGGGAGTCTCCATAGAATGGAGGAAAAGGAGATACAGCAC |
| | | ACAAGTAGACCCTGACCTAGCAGACCAACTAACTCATCTGTACTACTTTG |
| | | ATTGTTTTTCAGAATCTGCTATAAGAAATGCCATATTAGGACATAGAGTT |
| | | AGTCCTAGGTGTGAATATCAAGCAGGACATAACAAGGTAGGATCTCTACA |
| | | GTACTTGGCACTAGCAGCATTAGTAACACCAAGAAAGATAAAGCCACCTT |
| | | TGCCTAGTGTTGCGAAACTGACAGAGGACAGATGGAACAAGTCCCACAAG |
| | | ACCAAGGGCCACAGAGGGAGCCATACAATGAATGGACACTAAAGCTTTTA |
| | | GAGGAGCTTAAGAATGAAGCTGTCAGACATTTCCCTAGACCATGGCTTCA |
| | | TGGCCTAGGGCAATATATCTATGAAACTTATGAGGATACTTGGGCAGGAG |
| | | TGGAAGCCATAATAAGAATTCTGCAACAATTGCTGCTTATTCATTTCAGA |
| | | ATTGGGTGTCAACATAGCAGAATAGGCATTATTCGACAGAGGAGAACAAG |
| | | AAATGGAGCCAGTAGATCCTAGACTAGAGCCCTGGAAGCATCCAGGAAGT |
| | | CAGCCTAAGACTGCCTGTACCAATTGCTATTGCAAAAAGTGTTGCTTGCA |
| | | TTGCCAAGTTTGCTTCATAACAAAAGGCTTAGGCATCTCCTATGGCAGGA |
| | | AGAAGCGGAAAAAGCGACGAAGATCTCCTCAACACAGTCAGACTGATCAA |
| | | GCTTCTCTATCAAAGCAGTAAGTAGTACATGTAATGCAACCTTTAGTAAT |
| | | ATTAGCAATAGTAGCATTAGTAGTAGCACTAATAATAGTCATAGTTGTAT |
| | | GGTCCATTGTATTAATAGAATATAGAAAAATATTAAGACAAAAGAAAATA |
| | | GACAGGTTAATTGATAGAATAAGAGAAAAAGCAGAAGACAGTGGCAATGA |
| | | GAGTGATGGGGATCAGGAAGAATTATCAGCACTTGTGGAAAGGGGGCACC |
| | | TTGCTCCTTGGGATATTGATGATCTGTAGTGCTGCAGAACAATTGTGGGT |
| | | CACAGTCTATTATGGGGTACCTGTGTGGAAAGAAGCAAACACCACTCTAT |
| | | TTTGTGCATCAGATGCTAAAGCATATGATACAGAGGTACATAATGTTTGG |
| | | GCCACACATGCCTGTGTACCCACAGACCCCAACCCACAAGAAATACTATT |
| | | GGAAAATGTGACAGAAGATTTTAACATGTGGAAAAATAACATGGTAGAAC |
| | | AGATGCATGAGGATATAATCAGTTTATGGGATCAAAGTCTAAAGCCATGT |
| | | GTAAAATTAACCCCACTCTGTGTTACTTTACATTGCACTGATTTGAAGAA |
| | | TGGTACTAATTTGAAGAATGGTACTAAAATCATTGGGAAATCAATAAGAG |
| | | GAGAAATAAAAAACTGCTCTTTCAATGTCACCAAAAACATAATAGATAAG |
| | | GTGAAAAAAGAATATGCGCTTTTCTATAGACATGATGTAGTACCAATAGA |
| | | TAGGAATATTACTAGCTATAGGTTAATAAGTTGTAACACCTCAACCCTTA |
| | | CACAGGCCTGTCCAAAGGTATCCTTTGAGCCAATTCCCATACATTATTGT |
| | | GCCCCGGCTGGTTTTGCGATTCTAAAATGTAAAGATAAGAAGTTCAATGG |
| | | AACGGGACCATGTACAAATGTCAGTACAGTACAATGTACACATGGAATTA |
| | | GGCCAGTAGTATCAACTCAACTGCTGTTAAATGGAAGTCTAGCAGAAGAA |
| | | GAGGTAGTAATTAGATCTAGCAATTTCACGGACAATGCTAAAATCATAAT |
| | | AGTACAGCTGAATGAAACTGTAGAAATTAATTGTACAAGACCCAACAACA |
| | | ATACAAGAAAAGGGATAACTCTAGGACCAGGGAGAGTATTTTATACAACA |
| | | GGAAAAATAGTAGGAGATATAAGAAAAGCACATTGTAACATTAGTAAAGT |
| | | AAAATGGCATAACACTTTAAAAAGGGTAGTTAAAAAATTAAGAGAAAAAT |
| | | TTGAAAATAAAACAATAATCTTTAATAAATCCTCAGGGGGGGACCCAGAA |
| | | ATTGTAATGCACAGCTTTAATTGTGGAGGGGAATTTTTCTACTGTAATAC |
| | | AAAAAAACTGTTTAATAGTACTTGGAATGGTACTGAAGGGTCATATAACA |
| | | TTGAAGGAAATGACACTATCACACTCCCATGCAGAATAAAACAAATTATA |
| | | AACATGTGGCAGGAAGTAGGAAAAGCAATGTATGCCCCTCCCATCAGTGG |
| | | ACAAATTTGGTGCTCATCAAATATTACAGGGCTGCTACTAACAAGAGATG |
| | | GTGGTAAGAACAGCAGCACCGAAATCTTCAGACCTGGAGGAGGAGATATA |
| | | AGGGACAATTGGAGAAGTGAATTATATAAATATAAAGTAGTAAGAGTTGA |
| | | ACCATTAGGAATAGCACCCACCAAGGCAAAAAGAAGAGTGGTGCAGAGAG |
| | | AAAAAAGAGCAGTAGGAATAGGAGCTGTGTTCCTTGGGTTCTTGGGAGCA |
| | | GCAGGAAGCACTATGGGCGCAGCGTCAATAACGCTGACGGTACAGGCCAG |
| | | ACAATTATTGTCTGGTATAGTGCAACAGCAGAACAATTTGCTGAGGGCTA |
| | | TTGAAGCGCAACAGCATATGTTGCAACTCACAGTCTGGGGCATCAAGCAG |
| | | CTCCAGGCAAGAGTCCTGGCTGTGGAAAGATACCTACAGGATCAACAGCT |
| | | CCTGGGGATTTGGGGTTGCTCTGGAAAACTCATCTGCACCACTACTGTGC |
| | | CTTGGAATACTAGTTGGAGTAATAAATCTCTGGATACAATTTGGGGTAAC |
| | | ATGACCTCGATGCAGTGGGAAAAAGAAATTAACAATTACACAGGCTTAAT |
| | | ATACAACTTGATTGAAGAATCGCAGAACCAACAAGAAAAGAATGAACAAG |
| | | AATTATTGGCATTAGATAAATGGGCAAGTTTGTGGAATTGGTTTAACATA |
| | | TCAAACTGGCTGTGGTATATAAAAATATTCATAATGATAGTAGGAGGCTT |
| | | GATAGGTTTAAGAATAGTTTTCAGTGTACTTTCTATAGTGAATAGAGTTA |
| | | GGCAGGGATACTCACCATTATCGTTTCAGACCCGCTTCCCAGCCTCGAGG |
| | | GGACCCGACAGGCCCGAAGGAATCGAAGAAGAAGGTGGAGACAGAGACAG |
| | | AGACAGATCCAGTCCATTAGTGGATGGATTCTTAGCAATCATCTGGGTCG |
| | | ACCTGCGGAGCCTGTTCCTCTTCAGCTACCACCGCTTGAGAGACTTACTC |
| | | TTGATTGTAACGAGGATTGTGGAACTTCTGGGACGCAGGGGGTGGGAACT |
| | | CCTCAAATACTTGTGGAATCTCCTGCAGTATTGGAGTCAGGAACTAAAGA |
| | | ATAGTGCTGTTAGCTTGCTTAACGCCACAGCCATAGCAGTAGGTGAGGGA |
| | | ACAGATAGAATTATAGAAATATTACAAAGAGCTGGTAGAGCTATTCTCAA |
| | | CATACCTACGAGAATAAGACAGGGCTTAGAAAGGGCTTTGCTATAAGCTT |
| | | ATGGGTGGAGCTATTTCCATGAGGCGGTCCAGGCCGTCTGGAAATCTGTA |
| | | CGAAAGACTCTTGCGGGCGCGTGGGGAGACTTATGGAAAACTCTTAGGAG |
| | | AGGTAAAAGATGGATACTCGCAATCCCCAGGAGGATTAGACAAGGGCTTG |
| | | AGCTCACTCTCTTGTGAGGGACAAAAATACAATCAGGGACAGTATATGAA |
| | | TACTCCATGGAGAAACCCAGCTAAAGAGAGAGAAAAATTAGCATACAGAA |
| | | AACAAAATATGGATGATATAGATAAGGAAGATGATGACTTGGTAGGGGTA |
| | | TCAGTGAGGCCAAAAGTTCCCCTAAGAACAATGAGTTACAAATTGGCAAT |
| | | AGACATGTCTCATTTTATAAAAGAAAAGGGGGGACTGGAAGGGATTTATT |
| | | ACAGTGCAAGAAGACATAGAATCTTAGACATATACTTAGAAAAGGAAGAA |
| | | GGCATCATACCAGATTGGCAGGATTACACCTCAGGACCAGGAATTAGATA |
| | | CCCAAAGACATTTGGCTGGCTATGGAAATTAGTCCCTGTAAATGTATCAG |
| | | ATGAGGCACAGGAGGATGAGGAGCATTATTTAACGCATCCAGCTCAAACT |
| | | TCCCAGTGGGATGACCCTTGGGGAGAGGTTCTAGCATGGAAGTTTGATCC |
| | | AACTCTGGCCTACACTTATGAGGCATATGTTAGATACCCAGAAGAGTTTG |
| | | GAAGCAAGTCAGGCCTGTCAGAGGAAGAGGTTAAAAGAAGGCTAACCGCA |
| | | AGAGGCCTTCTTAACATGGCTGACAAGAAGGAAACTCGCTGAATTCGAGC |
| | | TATCTACAGGGGACTTTCCGCTGGGGACTTTCCAGGGAGGCGTGGCCTGG |
| | | GCGGGACCGGGGAGTGGCGAGCCCTCAGATGCTGCATATAAGCAGCCGCT |
| | | TTTGCCTGTACTGGGTCTCTCTAGTTAGACCAGATCTGAGCCTGGGAGCT |
| | | CTCTGGCTAGCTGAGAACCCACTGCTTAGGCCTCAATAAAGCTTGCCTTG |
| | | AGTGCTGTAAGTAGTGTGTGCCCGTCTGTTGTGTGACTCTGGTAACTAGA |
| | | GATCCCTCAGACCATTTTAGTCAGTGTGGAAAATCTCTAGCA |
| SEQ ID | clone 1.26 | TGGAAGGGATTTATTACAGTGCAAGAAGACATAGAATCTTAGACATATAC |
| NO: 6 | DNA | TTAGAAAAGGAAGAAGGCATCATACCAGATTGGCAGGATTACACCTCAGG |
| | | ACCAGGAATTAGATACCCAAAGACATTTGGCTGGCTATGGAAATTAGTCC |
| | | CTGTAAATGTATCAGATGAGGCACAGGAGGATGAGGAGCATTATTTAATG |
| | | CATCCAGCTCAAACTTCCCAGTGGGATGACCCTTGGGGAGAGGTTCTAGC |
| | | ATGGAAGTTTGATCCAACTCTGGCCTACACTTATGAGGCATACGTTAGAT |
| | | ACCCAGAAGAGTTTGGAAGCAAGTCAGGCCTGTCAGAGGAAGAGGTTAAA |
| | | AGAAGGCTAACCGCAAGAGGCCTTCTTAACATGGCTGACAAGAAGGAAAC |
| | | TCGCTGAATTCGAGCTATCTACAGGGGACTTTCCGCTGGGGACTTTCCAG |
| | | GGAGGCGTGGCCTGGGCGGGACCGGGGAGTGGCGAGCCCTCAGATGCTGC |
| | | ATATAAGCAGCCGCTTTTGCCTGTACTGGGTCTCTCTAGTTAGACCAGAT |
| | | CTGAGCCTGGGAGCTCTCTGGCTAGCTGAGAACCCACTGCTTAAGCCTCA |
| | | ATAAAGCTTGCCTTGAGTGCTTTAAGTAGTGTGTGCCCGTCTGTTGTGTG |
| | | ACTCTGGTAACTAGAGATCCCTCAGACCATTTTAGTCAGTGTGGAAAATC |
| | | TCTAGCAGTGGCGCCCGAACAGGGACCGGAAAGCGAAAGAGAAACCAGAG |
| | | AAGCTCTCTCGACGCAGGACTCGGCTTGCTGAAGCGCGCACGGCAAGCGG |
| | | CGAGGGGCAGCGACCGGTGAGTACGCTAAAAATTTTGACTAGCGGAGGCT |
| | | AGAAGGAGAGAGATGGGTGCGAGAGCGTCAGTATTAAGCGGGGGAAAATT |
| | | GGATGCATGGGAAAAAATTCGGTTACGGCCAGGAGGAAAGAAAAAATATA |
| | | GACTAAAACATCTAGTATGGGCAAGCAGGGAGCTAGAACGATTTGCACTT |
| | | AATCCTGGCCTTTTAGAGACATCAGATGGCTGTAAACAAATAATAGGACA |
| | | GCTACAACCAGCTATCCGGACAGGATCAGAAGAACTTAGATCATTATTTA |
| | | ATACAGTAGCAACCCTCTATTGTGTACATGAAAGGATAGAGGTAAAAGAC |
| | | ACCAAGGAAGCTTTAGAGAAGATAGAGGAAGAGCAAAACAAAAGTAAGAA |
| | | AAAAGCACAGCAAGCAGCAGCTGACACAGGACACAGCAATCAGGTCAGCC |
| | | AAAATTACCCTATAGTGCAGAACATCCAGGGGCAAATGGTACATCAGGCC |
| | | CTATCACCTAGAACTTTAAATGCGTGGGTAAAAGTAGTAGAAGAGAAGGC |
| | | TTTTAGCCCAGAAGTAATACCCATGTTTTCAGCATTATCAGAAGGAGCCA |
| | | CCCCACAAGATTTAAACACCATGCTAAACACAGTGGGGGGACATCAAGCA |
| | | GCCATGCAAATGTTAAAAGAGACCATCAATGAGGAAGCTGCAGAATGGGA |
| | | TAGAGTGCATCCAGTGCATGCAGGGCCTATTGCACCAGGCCAGATGAGAG |
| | | AACCAAGGGGAAGTGACATAGCAGGAACTACTAGTACCCTTCAGGAACAA |
| | | ATAGGATGGATGACACATAATCCACCTATCCCAGTAGGAGAAATCTATAA |
| | | AAGATGGATAATCCTGGGATTAAATAAAATAGTAAGAATGTATAGCCCTA |
| | | CCAGCATTCTGGACATAAGACAAGGACCAAAGGAACCCTTTAGAGACTAT |
| | | GTAGACCGGTTCTATAAAACCCTAAGAGCCGAGCAAGCTACACAGGAGGT |
| | | AAAAAATTGGATGACAGAAACCTTGTTGGTCCAAAATGCGAACCCAGATT |
| | | GTAAAACTATTTTAAAAGCATTGGGACCAGCAGCCACACTAGAAGAAATG |
| | | ATGACAGCATGTCAGGGAGTGGGAGGACCCGGCCATAAAGCAAGAGTTTT |
| | | GGCTGAAGCAATGAGCCAAGTAACAAATTCAGCTACCATAATGATGCAGA |
| | | GAGGCAATTTTAGGAACCAAAGAAAAACTGTTAAGTGTTTCAATTGTGGC |
| | | AAAGAAGGGCACATAGCCAAAAATTGCAGGGCTCCTAGGAAAAAGGGCTG |
| | | TTGGAAATGTGGAAAGGAAGGACACCAAATGAAAGATTGTACTGAGAGAC |
| | | AGGCTAATTTTTTAGGGAAGATCTGGCCTTCCCACAAGGGAAGGCCAGGA |
| | | AATTTTCTTCAGAGCAGACCAGAGCCAACAGCCCCATCAGAAGAGAGCGT |
| | | CAAGTTTGGAGAAGAGACAACAACTCCCTCTCAGAAGCAGGAGCCGATAG |
| | | ACAAGGAACTGTATCCTTTAACTTCCCTCAGATCACTCTTTGGCAACGAC |
| | | CCCTCGTCACAATAAAGATAGGGGGGCAACTAAAGGAAGCTCTATTAGAT |
| | | ACAGGAGCAGATGATACAGTATTAGAAGACATGGATTTGCCAGGAAGATG |
| | | GAAACCAAAAATGATAGGGGGAATTGGAGGTTTTATCAAAGTAAGACAGT |
| | | ACGATCAGATACCCATAGATATCTGTGGACATAAAGCTGTAGGTACAGTA |
| | | TTAGTAGGACCTACACCTGTCAACATAATTGGAAGAAATCTGTTGACTCA |
| | | GATTGGTTGCACTTTAAATTTTCCCATTAGTCCTATTGAAACTGTACCAG |
| | | TAAAATTAAAGCCAGGAATGGATGGCCCAAAAGTCAAACAATGGCCATTG |
| | | ACAGAAGAAAAAATAAAAGCATTAGTAGAAATTTGTACAGAAATGGAAAA |
| | | GGAAGGAAAGATTTCAAAAATTGGGCCTGAAAATCCATACAATACTCCAG |
| | | TATTTGCCATAAAGAAAAAAGACAGTACTAAATGGAGAAAATTAGTAGAT |
| | | TTCAGAGAACTTAATAGGAAAACTCAAGACTTCTGGGAAGTTCAATTAGG |
| | | AATACCACATCCCGCAGGGTTAAAAAAGAAAAAATCAGTAACAGTACTGG |
| | | ATGTGGGTGATGCATATTTTTCAGTTCCCTTAGATAAAGACTTCAGGAAG |
| | | TATACTGCATTTACCATACCTAGTATAAACAATGAGACACCAGGGATTAG |
| | | ATATCAGTACAATGTGCTTCCACAGGGATGGAAAGGATCACCAGCAATAT |
| | | TCCAAAGTAGCATGACAAAAACCTTAGAGCCTTTTAGAAAACAAAATCCA |
| | | GACATAATTATCTATCAATACATGGATGATTTGTATGTAGGATCTGACTT |
| | | AGAAATAGGGCAGCATAGAACAAAAATAGAGGAACTGAGACAACATCTGT |
| | | TAAAGTGGGGATTTACCACACCAGACAAAAAACATCAGAAAGAACCTCCA |
| | | TTCCTTTGGATGGGTTATGAACTCCATCCTGATAAATGGACAGTACAGCC |
| | | TATAGTGCTGCCAGAAAAAGACAGCTGGACTGTCAATGACATACAGAAGT |
| | | TAGTGGGAAAATTAAATTGGGCAAGTCAAATTTATGCAGGGATTAAAGTA |
| | | AAGCAATTATGTAAACTCCTTAGGGGAACCAAAGCACTTACAGAAGTAAT |
| | | ACCACTAACAAAAGAAGCAGAGCTAGAACTGGCAGAAAACAGGGAGATTT |
| | | TAAAGGAACCAGTACATGGAGTGTATTATGACCCATCAAAAGACTTAATA |
| | | GTAGAAATACAGAAGCAGGGGCAAGGCCAATGGACATATCAAATTTTTCA |
| | | AGAGCCATTTAAAAATCTGAAAACAGGAAAATATGCAAAAACGAGGGGTG |
| | | CCCACACTAATGATGTAAAACAATTAACAGAGGCAGTGCAAAAAATAGCC |
| | | AATGAAAGCATAGTAATATGGGGAAAGATTCCTAAATTTAAATTACCCAT |
| | | ACAAAAAGAAACATGGGAAACATGGTGGACAGAGTATTGGCAAGCCACCT |
| | | GGATTCCTGAGTGGGAGTTTGTCAATACCCCTCCCTTAGTGAAATTATGG |
| | | TACCAGTTAGAAAAAGAACCCATAGTAGGAGCAGAAACTTTCTATGTAGA |
| | | TGGGGCAGCTAACAGGGAGACTAAATTAGGAAAAGCAGGATATGTTACTA |
| | | GCAGAGGAAGGCAAAAAGTTGTCTCCCTAACAGACACAACAAATCAGAAA |
| | | ACTGAGTTACAAGCAATTCACCTAGCTTTGCAGGATTCAGGATTAGAAGT |
| | | AAACATAGTAACAGACTCACAATATGCATTAGGAATCATTCAAGCACAAC |
| | | CAGATAAAAGTGAATCAGAGTTAGTCAGTCAAATAATAGAACAGCTAATA |
| | | AAAAAGGAAAAAGTCTACCTGGCATGGGTACCAGCACACAAAGGAATTGG |
| | | AGGAAATGAACAGGTAGATAAATTAGTCAGTGCTGGAATCAGGAGAGTAC |
| | | TATTTCTAGATGGAATAGAGAAGGCCCAAGAAGAACATGAGAAATATCAT |
| | | AATAATTGGAGAGCAATGGCTAGTGAATTTAACCTGCCAGCTGTAGTAGC |
| | | AAAAGAGATAGTAGCCTGCTGTGATAAGTGCCAGGTAAAAGGAGAAGCCA |
| | | TGCATGGACAAGTAGACTGCAGTCCAGGAATATGGCAACTAGATTGTACA |
| | | CATTTAGAAGGAAAAGTTATCCTGGTAGCAGTTCATGTAGCCAGTGGATA |
| | | TATAGAAGCAGAGGTTATTCCAGCAGAGACAGGACAGGAGACAGCATACT |
| | | TTATTTTAAAATTAGCAGGAAGATGGCCAGTAAAAACAATACATACAGAC |
| | | AATGGCAGTAATTTCACCAGTACTACGGTTAAGGCCGCCTGTTGGTGGGC |
| | | AGGGATCAAGCAGGAATTTGGCATTCCCTACAATCCCCAAAGTCAAGGAG |
| | | TAGTAGAATCTATGAATAAAGAATTAAAGAAAATTATAGAACAAGTAAGA |
| | | GATCAGGCTGAACATCTTAAGACAGCAGTACAAATGGCAGTATTCATTCA |
| | | CAATTTTAAAAGAAAAGGGGGGATTGGGGGGTACAGTGCAGGGGAAAGAA |
| | | TAGTAGACATAATAGCATCAGACATACAAACTAAAGAACTACAAAAACAA |
| | | ATTACAAAAATTCAAAATTTTCGGGTTTATTACAGGGACAGCAGAGATCC |
| | | ACTTTGGAAAGGACCAGCAAAGCTTCTTTGGAAAGGTGAAGGGGCAGTAG |
| | | TAATACAAGATAAGAGTGACATAAAAGTAGTGCCAAGAAGAAAAGCAAAG |
| | | ATTATCAGGGATTATGGAAAACAGATGGCAGGTGATGATTGTGTGGCAAG |
| | | TAGACAGGATGAGGATTAGAACATGGAAAAGTTTAGTAAAACACCATATG |
| | | TATGTTTCAAAGAAAGCTAAGGGATGGTTTTATAGACATCACTATAAAAG |
| | | CACTCATCCAAGAATAAGTTCAGAAGTACATATCCCACTAGGGGATGCTA |
| | | GCTTGGTAGTAACAACATATTGGGGTCTACATACAGGAGAAAGAGACTGG |
| | | CATTTGGGTCAGGGAGTCTCCATAGAATGGAGGAAAAGGAGATACAGCAC |
| | | ACAAGTAGACCCTGACCTAGCAGACCAACTAATTCATCTGTACTACTTTG |
| | | ATTGTTTTTCAGAATCTGCTATAAGAAATGCCATATTAGGACATAGAGTT |
| | | AGTCCTAGGTGTGAATATCAAGCAGGACATAACAAGGTAGGATCTCTACA |
| | | GTACTTGGCACTAGCAGCATTAGTAACACCAAGAAAGATAAAGCCACCTT |
| | | TGCCTAGTGTTGCGAAACTGACAGAGGACAGATGGAACAAGTCCCACAAG |
| | | ACCAAGGGCCACAGAGGGAGCCATACAATGAATGGACACTAAAGCTTTTA |
| | | GAGGAGCTTAAGAATGAAGCTGTCAGACATTTCCCTAGACCATGGCTTCA |
| | | TGGCCTAGGGCAATATATCTATGAAACTTATGAGGATACTTGGGCAGGAG |
| | | TGGAAGCCATAATAAGAATTCTGCAACAATTGCTGCTTATTCATTTCAGA |
| | | ATTGGGTGTCAACATAGCAGAATAGGCATTATTCGACAGAGGAGAACAAG |
| | | AAATGGAGCCAGTAGATCCTAGACTAGAGCCCTGGAAGCATCCAGGAAGT |
| | | CAGCCTAAGACTGCCTGTACCAATTGCTATTGCAAAAAGTGTTGCTTGCA |
| | | TTGCCAAGTTTGCTTCATAACAAAAGGCTTAGGCATCTCCTATGGCAGGA |
| | | AGAAGCGGAAAAAGCGACGAAGATCTCCTCAACACAGTCAGACTGATCAA |
| | | GCTTCTCTATCAAAGCAGTAAGTAGTACATGTAATGCAACCTTTAGTAAT |
| | | ATTAGCAATAGTAGCATTAGTAGTAGCACTAATAATAGTCATAGTTGTAT |
| | | GGTCCATTGTATTAATAGAATATAGAAAAATATTAAGACAAAAGAAAATA |
| | | GACAGGTTAATTGATAGAATAAGAGAAAAAGCAGAAGACAGTGGCAATGA |
| | | GAGTGATGGGGATCAGGAAGAATTATCAGCACTTGTGGAAAGGGGGCACC |
| | | TTGCTCCTTGGGATATTGATGATCTGTAGTGCTGCAGAACAATTGTGGGT |
| | | CACAGTCTATTATGGGGTACCTGTGTGGAAAGAAGCAAACACCACTCTAT |
| | | TTTGTGCATCAGATGCTAAGGCATATGATACAGAGGTACATAATGTTTGG |
| | | GCCACACATGCCTGTGTACCCACAGACCCCAACCCACAAGAAATACTATT |
| | | GGAAAATGTGACAGAAGATTTTAACATGTGGAAAAATAACATGGTAGAAC |
| | | AGATGCATGAGGATATAATCAGTTTATGGGATCAAAGTCTAAAGCCATGT |
| | | GTAAAATTAACCCCACTCTGTGTTACTTTACATTGCACTGATTTGAAGAA |
| | | TGGTACTAATTTGAAGAATGGTACTAAAATCATTGGGAAATCAATAAGAG |
| | | GAGAAATAAAAAACTGCTCTTTCAATGTCACCAAAAACATAATAGATAAG |
| | | GTGAAAAAAGAATATGCGCTTTTCTATAGACATGATGTAGTACCAATAGA |
| | | TAGGAATATTACTAGCTATAGGTTAATAAGTTGTAACACCTCAACCCTTA |
| | | CACAGGCCTGTCCAAAGGTATCCTTTGAGCCAATTCCCATACATTATTGT |
| | | GCCCCGGCTGGTTTTGCGATTCTAAAATGTAAAGATAAGAAGTTCAATGG |
| | | AACGGGACCGTGTACAAATGTCAGTACAGTACAATGTACACATGGAATTA |
| | | GGCCAGTAGTATCAACTCAACTGCTGTTAAATGGAAGTCTAGCAGAAGGA |
| | | GAGGTAGTAATTAGATCTAGCAATTTCACGGACAATGCTAAAATCATAAT |
| | | AGTACAGCTGAATGAAGCTGTAGAAATTAATTGTACAAGACCCAACAACA |
| | | ATACAAGAAAAGGGATAACTCTAGGACCAGGGAGAGTATTTTATACAACA |
| | | GGAAAAATAGTAGGAGATATAAGAAAAGCACATTGTAACATTAGTAAAGT |
| | | AAAATGGCATAACACTTTAAAAAGGGTAGTTAAAAAATTAAGAGAAAAAT |
| | | TTGAAAATAAAACAATAATCTTTAATAAATCCTCAGGGGGGGACCCAGAA |
| | | ATTGTAATGCACAGCTTTAATTGTGGAGGGGAATTTTTCTACTGTAATAC |
| | | AAAAAAACTGTTTAATAGTACTTGGAATGGTACTGAAGGGTCATATAACA |
| | | TTGAAGGAAATGACACTATCACACTCCCATGCAGAATAAAACAAATTATA |
| | | AACATGTGGCAGGAAGTAGGAAAAGCAATGTATGCCCCTCCCATCAGTGG |
| | | ACAAATTTGGTGCTCATCAAATATTACAGGGCTGCTACTAACAAGAGATG |
| | | GTGGTAAGAACAGCAGCACCGAAATCTTCAGACCTGGAGGAGGAGATATA |
| | | AGGGACAATTGGAGAAGTGAATTATATAAATATAAAGTAGTAAGAGTTGA |
| | | ACCATTAGGAATAGCACCCACCAAGGCAAAAAGAAGAGTGGTGCAGAGAG |
| | | AAAAAAGAGCAGTGGGAATAGGAGCTGTGTTCCTTGGGTTCTTGGGAGCA |
| | | GCAGGAAGCACTATGGGCGCAGCGTCAATAACGCTGACGGTACAGGCCAG |
| | | ACAATTATTGTCTGGTATAGTGCAACAGCAGAACAATTTGCTGAGGGCTA |
| | | TTGAAGCGCAACAGCATATGTTGCAACTCACAGTCTGGGGCATCAAGCAG |
| | | CTCCAGGCAAGAGTCCTGGCTGTGGAAAGATACCTACAGGATCAACAGCT |
| | | CCTGGGGATTTGGGGTTGCTCTGGAAAACTCATCTGCACCACTACTGTGC |
| | | CTTGGAATACTAGTTGGAGTAATAAATCTCTGGATACAATTTGGGGTAAC |
| | | ATGACCTGGATGCAGTGGGAAAAAGAAATTAACAATTACACAGGCTTAAT |
| | | ATACAACTTGATTGAAGAATCGCAGAACCAACAAGAAAAGAATGAACAAG |
| | | AATTATTGGCATTAGATAAATGGGCAAGTTTGTGGAACTGGTTTAACATA |
| | | TCAAACTGGCTGTGGTATATAAAAATATTCATAATGATAGTAGGAGGCTT |
| | | GATAGGTTTAAGAATAGTTTTCAGTGTACTTTCTATAGTGAATAGAGTTA |
| | | GGCAGGGATACTCACCATTATCGTTTCAGACCCGCTTCCCAGCCTCGAGG |
| | | GGACCCGACAGGCCCGAAGGAATCGAAGAAGAAGGTGGAGACAGAGACAG |
| | | AGACAGATCCAGTCCATTAGTGGATGGATTCTTAGCAATCATCTGGGTCG |
| | | ACCTGCGGAGCCTGTTCCTCTTCAGCTACCACCGCTTGAGAGACTTACTC |
| | | TTGATTGTAACGAGGATTGTGGAACTTCTGGGACGCAGGGGGTGGGAACT |
| | | CCTCAAATACTTGTGGAATCTCCTGCAGTATTGGAGTCAGGAACTAAAGA |
| | | ATAGTGCTGTTAGCTTGCTTAACGCCACAGCCATAGCAGTAGGTGAGGGA |
| | | ACAGATAGAATTATAGAAATATTACAAAGAGCTGGTAGAGCTATTCTCAA |
| | | CATACCTACGAGAATAAGACAGGGCTTAGAAAGGGCTTTGCTATAAGCTT |
| | | ATGGGTGGAGCTATTTCCATGAGGCGGTCCAGGCCGTCTGGAAATCTGTA |
| | | CGAGAGACTCTTGCGGGCGCGTGGGGAGACTTATGGAAAACTCTTAGGAG |
| | | AGGTAAAAGATGGATACTCGCAATCCCCAGGAGGATTAGACAAGGGCTTG |
| | | AGCTCACTCTCTTGTGAGGGACAAAAATACAATCAGGGACAGTATATGAA |
| | | TACTCCATGGAGAAACCCAGCTAAAGAGAGAGAAAAATTAGCATACAGAA |
| | | AACAAAATATGGATGATATAAATAAGGAAGATGATGACTTGGTAGGGGTA |
| | | TCAGTGAGGCCAAAAGTTCCCCTAAGAACAATGAGTTACAAATTGGCAAT |
| | | AGACATGTCTCATTTTATAAAAGAAAAGGGGGGACTGGAAGGGATTTATT |
| | | ACAGTGCAAGAAGACATAGAATCTTAGACATATACTTAGAAAAGGAAGAA |
| | | GGCATCATACCAGATTGGCAGGATTACACCTCAGGACCAGGAATTAGATA |
| | | CCCAAAGACATTTGGCTGGCTATGGAAATTAGTCCCTGTAAATGTATCAG |
| | | ATGAGGCACAGGAGGATGAGGAGCATTATTTAATGCATCCAGCTCAAACT |
| | | TCCCAGTGGGATGACCCTTGGGGAGAGGTTCTAGCATGGAAGTTTGATCC |
| | | AACTCTGGCCTACACTTATGAGGCATATGTTAGATACCCAGAAGAGTTTG |
| | | GAAGCAAGTCAGGCCTGTCAGAGGAAGAGGTTAAAAGAAGGCTAACCGCA |
| | | AGAGGCCTTCTTAACATGGCTGACAAGAAGGAAACTCGCTGAATTCGAGC |
| | | TATCTACAGGGGACTTTCCGCTGGGGACTTTCCAGGGAGGCGTGGCCTGG |
| | | GCGGGACCGGGGAGTGGCGAGCCCTCAGATGCTGCATATAAGCAGCCGCT |
| | | TTTGCCTGTACTGGGTCTCTCTAGTTAGACCAGATCTGAGCCTGGGAGCT |
| | | CTCTGGCTAGCTGAGAACCCACTGCTTAAGCCTCAATAAAGCTTGCCTTG |
| | | AGTGCTTTAAGTAGTGTGTGCCCGTCTGTTGTGTGACTCTGGTAACTAGA |
| | | GATCCCTCAGACCATTTTAGTCAGTGTGGAAAATCTCTAGCA |
| SEQ ID | clone | TGGAAGGGATTTATTACAGTGCAAGAAGACATAGAATCTTAGACATATAC |
| NO: 7 | P8A26 DNA | TTAGAAAAGGAAGAAGGCATCATACCAGATTGGCAGGATTACACCTCAGG |
| | | ACCAGGAATTAGATACCCAAAGACATTTGGCTGGCTATGGAAATTAGTCC |
| | | CTGTAAATGTATCAGATGAGGCACAGGAGGATGAGGAGCATTATTTAATG |
| | | CATCCAGCTCAAACTTCCCAGTGGGATGACCCTTGGGGAGAGGTTCTAGC |
| | | ATGGAAGTTTGATCCAACTCTGGCCTACACTTATGAGGCATATGTTAAAT |
| | | ACCCAGAAGAGTTTGGAAGCAAGTCAGGCCTGTCAGAGGAAGAGGTTAGA |
| | | AGAAGGCTAACCGCAAGAGGCCTTCTTAACATGGCTGACAAGAAGGAAAC |
| | | TCGCTGAATTCGAGCTATCTACAGGGGACTTTCCGCTGGGGACTTTCCAG |
| | | GGAGGCGTGGCCTGGGCGGGACCGGGGAGTGGCGAGCCCTCAGATGCTGC |
| | | ATATAAGCAGCCGCTTTTGCCTGTACTGGGTCTCTCTAGTTAGACCAGAT |
| | | CTGAGCCTGGGAGCTCTCTGGCTAGCTGAGAACCCACTGCTTAAGCCTCA |
| | | ATAAAGCTTGCCTTGAGTGCTTTAAGTAGTGTGTGCCCGTCTGTTGTGTG |
| | | ACTCTGGTAACTAGAGATCCCTCAGACCATTTTAGTCAGTGTGGAAAATC |
| | | TCTAGCAGTGGCGCCCGAACAGGGACCGGAAAGCGAAAGAGAAACCAGAG |
| | | AAGCTCTCTCGACGCAGGACTCGGCTTGCTGAAGCGCGCACGGCAAGCGG |
| | | CGAGGGGCAGCGACCGGTGAGTACGCTAAAAATTTTGACTAGCGGAGGCT |
| | | AGAAGGAGAGAGATGGGTGCGAGAGCGTCAGTATTAAGCGGGGGAAAATT |
| | | GGATGCATGGGAAAAAATTCGGTTACGGCCAGGAGGAAAGAAAAAATATA |
| | | GACTAAAACATCTAGTATGGGCAAGCAGGGAGCTAGAACGATTTGCACTT |
| | | AATCCTGGCCTTTTAGAGACATCAGATGGCTGTAAACAAATAATAGGACA |
| | | GCTACAACCAGCTATCCGGACAGGATCAGAAGAACTTAGATCATTATTTA |
| | | ATACAGTAGCAACCCTCTATTGTGTACATGAAAGGATAGAGGTAAAAGAC |
| | | ACCAAGGAAGCTTTAGAGAAGATAGAGGAAGAGCAAAACAAAAGTAAGAA |
| | | AAAAGCACAGCAAGCAGCAGCTGACACAGGACACAGCAATCAGGTCAGCC |
| | | AAAATTACCCTATAGTGCAGAACATCCAGGGGCAAATGGTACATCAGGCC |
| | | CTATCACCTAGAACTTTAAATGCGTGGGTAAAAGTAGTAGAAGAGAAGGC |
| | | TTTTAGCCCAGAAGTAATACCCATGTTTTCAGCATTATCAGAAGGAGCCA |
| | | CCCCACAAGATTTAAACACCATGCTAAACACAGTGGGGGGACATCAAGCA |
| | | GCCATGCAAATGTTAAAAGAGACCATCAATGAGGAAGCTGCAGAATGGGA |
| | | TAGAGTGCATCCAGTGCATGCAGGGCCTATTGCACCAGGCCAGATGAGAG |
| | | AACCAAGGGGAAGTGACATAGCAGGAACTACTAGTACCCTTCAGGAACAA |
| | | ATAGGATGGATGACACATAATCCACCTATCCCAGTAGGAGAAATCTATAA |
| | | AAGATGGATAATCCTGGGATTAAATAAAATAGTAAGAATGTATAGCCCTA |
| | | CCAGCATTCTGGACATAAGACAAGGACCAAAGGAACCCTTTAGAGACTAT |
| | | GTAGACCGGTTCTATAAAACCCTAAGAGCCGAGCAAGCTACACAGGAGGT |
| | | AAAAAATTGGATGACAGAAACCTTGTTGGTCCAAAATGCGAACCCAGATT |
| | | GTAAAACTATTTTAAAAGCACTGGGACCAGCAGCTACACTAGAAGAAATG |
| | | ATGACAGCATGTCAGGGAGTGGGAGGACCCGGCCATAAAGCAAGAGTTTT |
| | | GGCTGAAGCAATGAGCCAAGTAACAAATTCAGCTACCATAATGATGCAGA |
| | | GAGGCAAATTTAGGAACCAAAGAAAAACTGTTAAGTGTTTCAATTGTGGC |
| | | AAAGAAGGGCACATAGCCAAAAATTGCAGGGCTCCTAGGAAAAAGGGCTG |
| | | TTGGAAATGTGGAAAGGAAGGACACCAAATGAAAGATTGTACTGAGAGAC |
| | | AGGCTAATTTTTTAGGGAAGATCTGGCCTTCCCACAAGGGAAGGCCAGGA |
| | | AATTTTCTTCAGAGCAGACCAGAGCCAACAGCCCCATCAGAAGAGAGCGT |
| | | CAGGTTTGGAGAGGAGACAACAACTCCCTCTCAGAAGCAGGAGCCGATAG |
| | | ACAAGGAACTGTATCCTTTAACTTCCCTCAGATCACTCTTTGGCAACGAC |
| | | CCCTCGTCACAATAAAGATAGGGGGGCAACTAAAGGAAGCTCTATTGGAT |
| | | ACAGGAGCAGATGATACAGTATTAGAAGACATGGATTTGCCAGGAAGATG |
| | | GAAACCAAAAATGATAGGGGGAATTGGAGGTTTTATCAAAGTAAGACAGT |
| | | ATGATCAGATACCCATAGATATCTGTGGACATAAAGCTGTAGGTACAGTA |
| | | TTAGTAGGACCTACACCTGTCAACATAATTGGAAGAAATCTGTTGACTCA |
| | | GATTGGTTGCACTTTAAATTTTCCCATTAGTCCTATTGAAACTGTACCAG |
| | | TAAAATTAAAGCCAGGAATGGATGGCCCAAAAGTCAAACAATGGCCATTG |
| | | ACAGAAGAAAAAATAAAAGCATTAGTAGAAATTTGTACAGAAATGGAAAA |
| | | GGAAGGAAAGATTTCAAAAATTGGGCCTGAAAATCCATACAATACTCCAG |
| | | TATTTGCCATAAAGAAAAAAGACAGTACTAAATGGAGAAAATTAGTAGAT |
| | | TTCAGAGAACTTAATAGGAAAACTCAAGACTTCTGGGAAGTTCAATTAGG |
| | | AATACCACATCCCGCAGGGTTAAAAAAGAAAAAATCAGTAACAGTACTGG |
| | | ATGTGGGTGATGCATATTTTTCAGTTCCCTTAGATAAAGACTTCAGGAAG |
| | | TATACTGCATTTACCATACCTAGTATAAACAATGAGACACCAGGGATTAG |
| | | ATATCAGTACAATGTGCTTCCACAGGGATGGAAAGGATCACCAGCAATAT |
| | | TCCAAAGTAGCATGACAAAAACCTTAGAGCCTTTTAGAAAACAAAATCCA |
| | | GACATAATTATCTATCAATACATGGATGATTTGTATGTAGGATCTGACTT |
| | | AGAAATAGGGCAGCATAGAACAAAAATAGAGGAACTGAGACAACATCTGT |
| | | TGAAGTGGGGATTTACCACACCAGACAAAAAACATCAGAAAGAACCTCCA |
| | | TTCCTTTGGATGGGTTATGAACTCCATCCTGATAAATGGACAGTACAGCC |
| | | TATAGTGCTGCCAGAAAAAGACAGCTGGACTGTCAATGACATACAGAAGT |
| | | TAGTGGGAAAATTAAATTGGGCAAGTCAAATTTATGCAGGGATTAAAGTA |
| | | AAGCAATTATGTAAACTCCTTAGGGGAACCAAAGCACTTACAGAAGTAAT |
| | | ACCACTAACAAAAGAAGCAGAGCTAGAACTGGCAGAAAACAGGGAGATTC |
| | | TAAAGGAACCAGTACATGGAGTGTATTATGACCCATCAAAAGACTTAATA |
| | | GTAGAAATACAGAAGCAGGGGCAAGGCCAATGGACATATCAAATTTTTCA |
| | | AGAGCCATTTAAAAATCTGAAAACAGGAAAATATGCAAAAACGAGGGGTG |
| | | CCCACACTAATGATGTAAAACAATTAACAGAGGCAGTGCAAAAAATAGCC |
| | | AATGAAAGCATAGTAATATGGGGAAAGATTCCTAAATTTAAATTACCCAT |
| | | ACAAAAAGAAACATGGGAAACATGGTGGACAGAGTATTGGCAAGCCACCT |
| | | GGATTCCTGAGTGGGAGTTTGTCAATACCCCTCCCTTAGTGAAATTATGG |
| | | TACCAGTTAGAAAAAGAACCCATAGTAGGAGCAGAAACTTTCTATGTAGA |
| | | TGGGGCAGCTAACAGGGAGACTAAATTAGGAAAAGCAGGATATGTTACTA |
| | | GCAGAGGAAGGCAAAAAGTTGTCTCCCTAACAGACACAACAAATCAGAAA |
| | | ACTGAGTTACAAGCAATTCACCTAGCTTTGCAGGATTCAGGATTAGAAGT |
| | | AAACATAGTAACAGACTCACAATATGCATTAGGAATCATTCAAGCACAAC |
| | | CAGATAAAAGTGAATCAGAGTTAGTCAGTCAAATAATAGAACAGCTAATA |
| | | AAAAAGGAAAAAGTCTACCTGACATGGATACCAGCACACAAAGGAATTGG |
| | | AGGAAATGAACAGGTAGATAAATTAGTCAGTGCTGGAATCAGGAGAGTAC |
| | | TATTTCTAGATGGAATAGAGAAGGCCCAAGAAGAACATGAGAAATATCAT |
| | | AGTAATTGGAGAGCAATGGCTAGTGAATTTAACCTGCCAGCTGTAGTAGC |
| | | AAAAGAAATAGTAGCCTGCTGTGATAAGTGCCAGGTAAAAGGAGAAGCCA |
| | | TGCATGGACAAGTAGACTGCAGTCCAGGAATATGGCAACTAGATTGTACA |
| | | CATTTAGAAGGAAAAGTTATCCTGGTAGCAGTTCATGTAGCCAGTGGATA |
| | | TATAGAAGCAGAGGTTATTCCAGCAGAGACAGGACAGGAAACAGCATACT |
| | | TTATTTTAAAATTAGCAGGAAGATGGCCAGTAAAAACAATACATACAGAC |
| | | AATGGCAGTAATTTCACCAGTACTACGGTTAAGGCCGCCTGTTGGTGGGC |
| | | AGGGATCAAGCAGGAATTTGGCATTCCCTACAATCCCCAAAGTCAAGGAG |
| | | TAGTAGAATCTATGAATAAAGAATTAAAGAAAATTATAGAACAAGTAAGA |
| | | GATCAGGCTGAACATCTTAAGACAGCAGTACAAATGGCAGTATTCATTCA |
| | | CAATTTTAAAAGAAAAGGGGGGATTGGGGGGTACAGTGCAGGGGAAAGAA |
| | | TAGTAGACATAATAGCATCAGACATACAAACTAAAGAACTACAAAAACAA |
| | | ATTACAAAAATTCAAAATTTTCGGGTTTATTACAGGGACAGCAGAGATCC |
| | | ACTTTGGAAAGGACCAGCAAAGCTTCTTTGGAAAGGTGAAGGGGCAGTAG |
| | | TAATACAAGATAAGAGTGACATAAAAGTAGTGCCAAGAAGAAAAGCAAAG |
| | | ATTATCAGGGATTATGGAAAACAGATGGCAGGTGATGATTGTGTGGCAAG |
| | | TAGACAGGATGAGGATTAGAACATGGAAAAGTTTAGTAAAACACCATATG |
| | | TATGTTTCAAAGAAAGCTAAGGGATGGTTTTATAGACATCACTATGAAAG |
| | | CACTCATCCAAGAATAAGTTCAGAAGTACATATCCCACTAGGGGATGCTA |
| | | GCTTGGTAGTAACAACATATTGGGGTCTACATACAGGAGAAAGAGACTGG |
| | | CATTTGGGTCAGGGAGTCTCCATAGAATGGAGGAAAAGGAGATACAGCAC |
| | | ACAAGTAGACCCTGACCTAGCAGACCAACTAATTCATCTGTACTACTTTG |
| | | ATTGTTTTTCAGAATCTGCTATAAGAAATGCCATATTAGGACATAGAGTT |
| | | AGTCCTAGGTGTGAATATCAAGCAGGACATAACAAGGTAGGATCTCTACA |
| | | GTACTTGGCACTAGCAGCATTAGTAACACCAAGAAAGATAAAGCCACCTT |
| | | TGCCTAGTGTTGCGAAACTGACAGAGGACAGATGGAACAAGTCCCACAAG |
| | | ACCAAGGGCCACAGAGGGAGCCATACAATGAATGGACACTAGAGCTTTTA |
| | | GAGGAGCTTAAGAATGAAGCTGTCAGACATTTCCCTAGACCATGGCTTCA |
| | | TGGCCTAGGACAATATATCTATGAAACTTATGAGGATACTTGGGCAGGAG |
| | | TGGAAGCCATAATAAGAATTCTGCAACAATTGCTGCTTATTCATTTCAGA |
| | | ATTGGGTGTCAACATAGCAGAATAGGCATTATTCGACAGAGGAGAACAAG |
| | | AAATGGAGCCAGTAGATCCTAGACTAGAGCCCTGGAAGCATCCAGGAAGT |
| | | CAGCCTAAGACTGCCTGTACCAATTGCTATTGCAAAAAGTGTTGCTTGCA |
| | | TTGCCAAGTTTGCTTCATAACAAAAGGCTTAGGCATCTCCTATGGCAGGA |
| | | AGAAGCGGAAAAAGCGACGAAGATCTCCTCAACACAGTCAGACTGATCAA |
| | | GCTTCTCTATCAAAGCAGTAAGTAGTACATGTAATGCAACCTTTGGTAAT |
| | | ATTAGCAATAGTAGCATTAGTAGTAGCACTAATAATAGTCATAGTTGTAT |
| | | GGTCCATTGTATTAATAGAATATAGAAAAATATTAAGACAAAAGAAAATA |
| | | GACAGGTTAATTGATAGAATAAGAGAAAGAGCAGAAGACAGTGGCAATGA |
| | | GAGTGATGGGGATCAGGAAGAATTATCAGCACTTGTGGAAAGGGGGCACC |
| | | TTGCTCCTTGGAATATTGATGATCTGTAGTGCTGCAGAACAATTGTGGGT |
| | | CACAGTCTATTATGGGGTACCTGTGTGGAAAGAAGCAAACACCACTCTAT |
| | | TTTGTGCATCAGATGCTAAAGCATATGATACAGAGGTACATAATGTTTGG |
| | | GCCACACATGCCTGTGTACCCACAGACCCCAACCCACAAGAAATACTATT |
| | | GGAAAATGTGACAGAAGATTTTAACATGTGGAAAAATAACATGGTAGAAC |
| | | AGATGCATGAGGATATAATCAGTTTATGGGATCAAAGTCTAAAGCCATGT |
| | | GTAAAATTAACCCCACTCTGTGTTACTTTACATTGCACTGATTTGAAGAA |
| | | TGGTACTAATTTGAAGAATGGTACTAAAATCATTGGGAAATCAATGAGAG |
| | | GAGAAATAAAAAACTGCTCTTTCAATGTCACCAAAAACATAATAGATAAG |
| | | GTGAAAAAAGAATATGCGCTTTTCTATAGACATGATGTAGTACCAATAGA |
| | | TAGGAATATTACTAGCTATAGGTTGATAAGTTGTAACACCTCAACCCTTA |
| | | CACAGGCCTGTCCAAAGGTATCCTTTGAGCCAATTCCCATACATTATTGT |
| | | GCCCCGGCTGGTTTTGCGATTCTAAAATGTAAAGATAAGAAGTTCAATGG |
| | | AACGGGACCATGTACAAATGTCAGTACAGTACAATGTACACATGGAATTA |
| | | GGCCAGTAGTATCAACTCAACTGCTGTTAAATGGAAGTCTAGCAGAAGAA |
| | | GAGGTAGTAATTAGATCTAGCAATTTCACGGACAATGCTAAAATCATAAT |
| | | AGTACAGCTGAATGAAACTGTAGAAATTAATTGTACAAGACCCAACAACA |
| | | ATACAAGAAAAGGGATAACTCTAGGACCAGGGAGAGTATTTTATACAACA |
| | | GGAAAAATAGTAGGAGATATAAGAAAAGCACATTGTAACATTAGTAAAGT |
| | | AAAATGGCATAACACTTTAAAAAGGGTAGTTGAAAAATTAAGAGAAAAAT |
| | | TTGAAAATAAAACAATAATCTTTAATAAATCCTCAGGGGGGGACCCAGAA |
| | | ATTGTAATGCACAGCTTTAATTGTGGAGGGGAATTTTTCTACTGTAATAC |
| | | AAAAAAACTGTTTAATAGTACTTGGAATGGTACTGAAGGGTCATATAACA |
| | | TTGAAGGAAATGACACTATCACACTCCCATGCAGAATAAAACAAATTATA |
| | | AACATGTGGCAGGAAGTAGGAAAAGCAATGTATGCCCCTCCCATCAGTGG |
| | | ACAAATTTGGTGCTCATCAAATATTACAGGGCTGCTACTAACAAGAGATG |
| | | GTGGTAAGAACAGCAGCACCGAAATCTTCAGACCTGGAGGAGGAGATATA |
| | | AGGGACAATTGGAGAAGTGAATTATATAAATATAAAGTAGTAAGAGTTGA |
| | | ACCATTAGGAATAGCACCCACCAAGGCAAAGAGAAGAGTGGTGCAGAGAG |
| | | AAAAAAGAGCAGTGGGAATAGGAGCTGTGTTCCTTGGGTTCTTGGGAGCA |
| | | GCAGGAAGCACTATGGGCGCAGCGTCAATAACGCTGACGGTACAGGCCAG |
| | | ACAATTATTGTCTGGTATAGTGCAACAGCAGAACAATTTGCTGAGGGCTA |
| | | TTGAAGCGCAACAGCATATGTTGCAACTCACAGTCTGGGGCATCAAGCAG |
| | | CTCCAGGCAAGAGTCCTGGCTGTGGAAAGATACCTACAGGATCAACAGCT |
| | | CCTGGGGATTTGGGGTTGCTCTGGAAAACTCATCTGCACCACTACTGTGC |
| | | CTTGGAATACTAGTTGGAGTAATAAATCTCTGGATACAATTTGGGGTAAC |
| | | ATGACCTGGATGCAGTGGGAAAAAGAAATTAACAATTACACAGGCTTAAT |
| | | ATACAACTTGATTGAAGAATCGCAGAACCAACAAGAAAAGAATGAACAAG |
| | | AATTATTGGCATTAGATAAATGGGCAAGTTTGTGGAATTGGTTTAACATA |
| | | TCAAACTGGCTGTGGTATATAAAAATATTCATAATGATAGTAGGAGGCTT |
| | | GATAGGTTTAAGAATAGTTTTCAGTGTACTTTCTATAGTGAATAGAGTTA |
| | | GGCAGGGATACTCACCATTATCGTTTCAGACCCGCTTCCCAGCCTCGAGG |
| | | GGACCCGACAGGCCCGAAGGAATCGAAGAAGAAGGTGGAGACAGAGACAG |
| | | AGACAGATCCAGTCCATTAGTGGATGGATTCTTAGCAATCATCTGGGTCG |
| | | ACCTGCGGAGCCTGTTCCTCTTCAGCTACCACCGCTTGAGAGACTTACTC |
| | | TTGATTGTAACGAGGATTGTGGAACTTCTGGGACGCAGGGGGTGGGAACT |
| | | CCTCAAATACTTGTGGAATCTCCTGCAGTATTGGAGTCAGGAACTAAAGA |
| | | ATAGTACTGTTAGCTTGCTTAACGCCACAGCCATAGCAGTAGGTGAGGGA |
| | | ACAGATAGGATTATAGAAATATTACAAAGAGCTGGTAGAGCTATTCTCAA |
| | | CATACCTACGAGAATAAGACAGGGCTTAGAAAGGGCTTTGCTATAAGCTT |
| | | ATGGGTGGAGCTATTTCCATGAGGCGGTCCAGGCCGTCTGGAGATCTGTA |
| | | CGAGAGACTCTTGCGGGCGCGTGGGGAGACTTATGGGAGACTCTTAGGAG |
| | | AGGTGGAAGATGGATACTCGCAATCCCCAGGAGGATTAGACAAAGGCTTG |
| | | AGCTCACTCTCTTGTGAGGGACAGAAATACAATCAGGGACAGTATATGAA |
| | | TACTCCATGGAGAAACCCAGCTAAAGAGAAAGAAAAATTAGCATACAGAA |
| | | AACAAAATATGAATGATATAAATAAGGAAGATGATAACTTGGTAGGGGTA |
| | | TCAGTGAGGCCAAAAGTTCCCCTAAGAACAATGAGTTACAAATTGGCAAT |
| | | AGACATGTCTCATTTTATAAAAGAAAAGGGGGGACTGGAAGGGATTTATT |
| | | ACAGTGCAAGAAGACATAGAATCTTAGACATATACTTAGAAAAGGAAGAA |
| | | GGCATCATACCAGATTGGCAGGATTACACCTCAGGACCAGGAATTAGATA |
| | | CCCAAAGACATTTGGCTGGCTATGGAAATTAGTCCCTGTAAATGTATCAG |
| | | ATGAGGCACAGGAGGATGAGGAGCATTATTTAATGCATCCAGCTCAAACT |
| | | TCCCAGTGGGATGACCCTTGGGGAGAGGTTCTAGCATGGAAGTTTGATCC |
| | | AACTCTGGCCTACACTTATGAGGCATATGTTAAATACCCAGAAGAGTTTG |
| | | GAAGCAAGTCAGGCCTGTCAGAGGAAGAGGTTAGAAGAAGGCTAACCGCA |
| | | AGAGGCCTTCTTAACATGGCTGACAAGAAGGAAACTCGCTGAATTCGAGC |
| | | TATCTACAGGGGACTTTCCGCTGGGGACTTTCCAGGGAGGCGTGGCCTGG |
| | | GCGGGACCGGGGAGTGGCGAGCCCTCAGATGCTGCATATAAGCAGCCGCT |
| | | TTTGCCTGTACTGGGTCTCTCTAGTTAGACCAGATCTGAGCCTGGGAGCT |
| | | CTCTGGCTAGCTGAGAACCCACTGCTTAAGCCTCAATAAAGCTTGCCTTG |
| | | AGTGCTTTAAGTAGTGTGTGCCCGTCTGTTGTGTGACTCTGGTAACTAGA |
| | | GATCCCTCAGACCATTTTAGTCAGTGTGGAAAATCTCTAGCA |
| SEQ ID | clone 1.4 | MGARASVLSGGKLDAWEKIRLRPGGKKKYRLKHLVWASRELERFALNPGL |
| NO: 8 | protein | LETSDGCKQIIGQLQPAIRTGSEELRSLFNTVATLYCVHERIEVKDTKEA |
| | Gag; | LEKIEEEQNKSKKKAQQAAADTGHSNQVSQNYPIVQNIQGQMVHQALSPR |
| | clone 1.26 | TLNAWVKVVEEKAFSPEVIPMFSALSEGATPQDLNTMLNTVGGHQAAMQM |
| | protein | LKETINEEAAEWDRVHPVHAGPIAPGQMREPRGSDIAGTTSTLQEQIGWM |
| | Gag; | THNPPIPVGEIYKRWIILGLNKIVRMYSPTSILDIRQGPKEPFRDYVDRF |
| | clone 1.27 | YKTLRAEQATQEVKNWMTETLLVQNANPDCKTILKALGPAATLEEMMTAC |
| | protein | QGVGGPGHKARVLAEAMSQVTNSATIMMQRGNFRNQRKTVKCFNCGKEGH |
| | Gag | IAKNCRAPRKKGCWKCGKEGHQMKDCTERQANFLGKIWPSHKGRPGNFLQ |
| | | SRPEPTAPSEESVKFGEETTTPSQKQEPIDKELYPLTSLRSLFGNDPSSQ |
| SEQ ID | clone 1.4 | FFREDLAFPQGKARKFSSEQTRANSPIRRERQVWRRDNNSLSEAGADRQG |
| NO: 9 | protein | TVSFNFPQITLWQRPLVTIKIGGQLKEALLDTGADDTVLEDMDLPGRWKP |
| | Pol; | KMIGGIGGFIKVRQYDQIPIDICGHKAVGTVLVGPTPVNIIGRNLLTQIG |
| | clone 1.26 | CTLNFPISPIETVPVKLKPGMDGPKVKQWPLTEEKIKALVEICTEMEKEG |
| | protein | KISKIGPENPYNTPVFAIKKKDSTKWRKLVDFRELNRKTQDFWEVQLGIP |
| | Pol; | HPAGLKKKKSVTVLDVGDAYFSVPLDKDFRKYTAFTIPSINNETPGIRYQ |
| | clone | YNVLPQGWKGSPAIFQSSMTKTLEPFRKQNPDIIIYQYMDDLYVGSDLEI |
| | P10.21 | GQHRTKIEELRQHLLKWGFTTPDKKHQKEPPFLWMGYELHPDKWTVQPIV |
| | protein | LPEKDSWTVNDIQKLVGKLNWASQIYAGIKVKQLCKLLRGTKALTEVIPL |
| | Pol; | TKEAELELAENREILKEPVHGVYYDPSKDLIVEIQKQGQGQWTYQIFQEP |
| | clone | FKNLKTGKYAKTRGAHTNDVKQLTEAVQKIANESIVIWGKIPKFKLPIQK |
| | P10.26 | ETWETWWTEYWQATWIPEWEFVNTPPLVKLWYQLEKEPIVGAETFYVDGA |
| | protein | ANRETKLGKAGYVTSRGRQKVVSLTDTTNQKTELQAIHLALQDSGLEVNI |
| | Pol | VTDSQYALGIIQAQPDKSESELVSQIIEQLIKKEKVYLAWVPAHKGIGGN |
| | | EQVDKLVSAGIRRVLFLDGIEKAQEEHEKYHNNWRAMASEFNLPAVVAKE |
| | | IVACCDKCQVKGEAMHGQVDCSPGIWQLDCTHLEGKVILVAVHVASGYIE |
| | | AEVIPAETGQETAYFILKLAGRWPVKTIHTDNGSNFTSTTVKAACWWAGI |
| | | KQEFGIPYNPQSQGVVESMNKELKKIIEQVRDQAEHLKTAVQMAVFIHNF |
| | | KRKGGIGGYSAGERIVDIIASDIQTKELQKQITKIQNFRVYYRDSRDPLW |
| | | KGPAKLLWKGEGAVVIQDKSDIKVVPRRKAKIIRDYGKQMAGDDCVASRQ |
| | | DED |
| SEQ ID | clone 1.4 | MENRWQVMIVWQVDRMRIRTWKSLVKHHMYVSKKAKGWFYRHHYESTHPR |
| NO: 10 | protein | ISSEVHIPLGDASLVVTTYWGLHTGERDWHLGQGVSIEWRKRRYSTQVDP |
| | Vif; | DLADQLIHLYYFDCFSESAIRNAILGHRVSPRCEYQAGHNKVGSLQYLAL |
| | clone 1.10 | AALVTPRKIKPPLPSVAKLTEDRWNKSHKTKGHRGSHTMNGH |
| | protein | |
| | Vif; | |
| | clone 1.27 | |
| | protein | |
| | Vif; | |
| | clone | |
| | P8A26 | |
| | protein | |
| | Vif | |
| SEQ ID | clone 1.4 | MEQVPQDQGPQREPYNEWTLKLLEELKNEAVRHFPRPWLHGLGQYIYETY |
| NO: 11 | protein | EDTWAGVEAIIRILQQLLLIHFRIGCQHSRIGIIRQRRTRNGASRS |
| | Vpr; | |
| | clone | |
| | P10.21 | |
| | protein | |
| | Vpr; | |
| | clone 1.26 | |
| | protein | |
| | Vpr; | |
| | clone 1.10 | |
| | protein | |
| | Vpr; | |
| | clone 1.27 | |
| | protein | |
| | Vpr; | |
| | clone | |
| | P10.26 | |
| | protein | |
| | Vpr | |
| SEQ ID | clone 1.4 | MEPVDPRLEPWKHPGSQPKTACTNCYCKKCCLHCQVCFITKGLGISYGRK |
| NO: 12 | protein | KRKKRRRSPQHSQTDQASLSKQPASQPRGDPTGPKESKKKVETETETDPVH |
| | Tat | |
| | clone | |
| | P10.21 | |
| | protein | |
| | Tat | |
| | clone 1.26 | |
| | protein | |
| | Tat | |
| | clone 1.10 | |
| | protein | |
| | Tat | |
| | clone 1.27 | |
| | protein | |
| | Tat | |
| | clone | |
| | P10.26 | |
| | protein | |
| | Tat | |
| | clone | |
| | P8A26 | |
| | protein | |
| | Tat | |
| SEQ ID | clone 1.4 | MAGRSGKSDEDLLNTVRLIKLLYQSNPLPSLEGTRQARRNRRRRWRQRQR |
| NO: 13 | protein | QIQSISGWILSNHLGRPAEPVPLQLPPLERLTLDCNEDCGTSGTQGVGTP |
| | Rev | QILVESPAVLESGTKE |
| | clone | |
| | P10.21 | |
| | protein | |
| | Rev | |
| | clone 1.26 | |
| | protein | |
| | Rev | |
| | clone 1.27 | |
| | protein | |
| | Rev | |
| | clone | |
| | P10.26 | |
| | protein | |
| | Rev | |
| | clone | |
| | P8A26 | |
| | protein | |
| | Rev | |
| SEQ ID | clone 1.4 | MQPLVILAIVALVVALIIVIVVWSIVLIEYRKILRQKKIDRLIDRIREKA |
| NO: 14 | protein | EDSGNESDGDQEELSALVERGHLAPWDIDDL |
| | Vpu; | |
| | clone | |
| | P10.21 | |
| | protein | |
| | Vpu; | |
| | clone 1.26 | |
| | protein | |
| | Vpu; | |
| | clone 1.10 | |
| | protein | |
| | Vpu; | |
| | clone 1.27 | |
| | protein | |
| | Vpu; | |
| | clone | |
| | P10.26 | |
| | protein | |
| | Vpu | |
| SEQ ID | clone 1.4 | MRVMGIRKNYQHLWKGGTLLLGILMICSAAEQLWVTVYYGVPVWKEANTT |
| NO: 15 | protein | LFCASDAKAYDTEVHNVWATHACVPTDPNPQEILLENVTEDFNMWKNNMV |
| | Env; | EQMHEDIISLWDQSLKPCVKLTPLCVTLHCTDLKNGTNLKNGTKIIGKSI |
| | clone 1.27 | RGEIKNCSFNVTKNIIDKVKKEYALFYRHDVVPIDRNITSYRLISCNTST |
| | protein | LTQACPKVSFEPIPIHYCAPAGFAILKCKDKKFNGTGPCTNVSTVQCTHG |
| | Env; | IRPVVSTQLLLNGSLAEEEVVIRSSNFTDNAKIIIVQLNETVEINCTRPN |
| | clone | NNTRKGITLGPGRVFYTTGKIVGDIRKAHCNISKVKWHNTLKRVVKKLRE |
| | P10.26 | KFENKTIIFNKSSGGDPEIVMHSFNCGGEFFYCNTKKLFNSTWNGTEGSY |
| | protein | NIEGNDTITLPCRIKQIINMWQEVGKAMYAPPISGQIWCSSNITGLLLTR |
| | Env | DGGKNSSTEIFRPGGGDIRDNWRSELYKYKVVRVEPLGIAPTKAKRRVVQ |
| | | REKRAVGIGAVFLGFLGAAGSTMGAASITLTVQARQLLSGIVQQQNNLLR |
| | | AIEAQQHMLQLTVWGIKQLQARVLAVERYLQDQQLLGIWGCSGKLICTTT |
| | | VPWNTSWSNKSLDTIWGNMTWMQWEKEINNYTGLIYNLIEESQNQQEKNE |
| | | QELLALDKWASLWNWFNISNWLWYIKIFIMIVGGLIGLRIVFSVLSIVNR |
| | | VRQGYSPLSFQTRFPASRGPDRPEGIEEEGGDRDRDRSSPLVDGFLAIIW |
| | | VDLRSLFLFSYHRLRDLLLIVTRIVELLGRRGWELLKYLWNLLQYWSQEL |
| | | KNSAVSLLNATAIAVGEGTDRIIEILQRAGRAILNIPTRIRQGLERALL |
| SEQ ID | clone 1.4 | MGGAISMRRSRPSGNLYERLLRARGETYGKLLGEVKDGYSQSPGGLDKGL |
| NO: 16 | protein | SSLSCEGQKYNQGQYMNTPWRNPAKEREKLAYRKQNMDDIDKEDDDLVGV |
| | Nef | SVRPKVPLRTMSYKLAIDMSHFIKEKGGLEGIYYSARRHRILDIYLKKEE |
| | | GIIPDWQDYTSGPGIRYPKTFGWLWKLVPVNVSDEAQEDEEHYLMHPAQT |
| | | SQWDDPWGEVLAWKFDPTLAYTYEAYVRYPEEFGSKSGLSEEEVKRRLTA |
| | | RGLLNMADKKETR |
| SEQ ID | clone | MGARASVLSGGKLDAWEKIRLRPGGKKKYRLKHLVWASRELERFALNPGL |
| NO: 17 | P10.26 | LETSDGCKQIIGQLQPAIRTGSEELRSLFNTVATLYCVHERIKVKDTKEA |
| | protein | LEKIEEEQNKSKKKAQQAAADTGHSNQVSQNYPIVQNIQGQMVHQALSPR |
| | Gag | TLNAWVKVVEEKAFSPEVIPMFSALSEGATPQDLNTMLNTVGGHQAAMQM |
| | | LKETINEEAAEWDRVHPVHAGPIAPGQMREPRGSDIAGTTSTLQEQIGWM |
| | | THNPPIPVGEIYKRWIILGLNKIVRMYSPTSILDIRQGPKEPFRDYVDRF |
| | | YKTLRAEQATQEVKNWMTETLLVQNANPDCKTILKALGPAATLEEMMTAC |
| | | QGVGGPGHKARVLAEAMSQVTNSATIMMQRGNFRNQRKTVRCFNCGKEGH |
| | | IAKNCRAPRKKGCWKCGKEGHQMKDCTERQANFLGKIWPSHKGRPGNFPQ |
| | | SRPEPTAPSEESVKFGEETTTPSQKQEPIDKELYPLTSLRSLFGNDPSSQ |
| SEQ ID | clone | MENRWQVMIVWQVDRMRIRTWKSLVKHHMYVSKKAKGWFYRHHYESTHPR |
| NO: 18 | P10.26 | ISSEVHIPLGDASLVVTTYWGLHTGERDWHLGQGVSIEWRKRRYSTQVDP |
| | protein | DLADQLIHLYYFDCFSESAIRNAILGHRVSPRCEYQAGHNKVGSLQYLAL |
| | Vif | AALVTPRKIKPPLPSVAKLTEDRWNKSHKTRGHRGSHTMNGH |
| SEQ ID | clone | MGGAISMRRSRPSGNLYERLLRARGETYGKLLGEVKDGYSQSPGGLDKGL |
| NO: 19 | P10.26 | SSLSCEGQKYNQGQYMNTPWRNPAKEREKLAYRKQNMDDIDKEDDDLVGV |
| | protein | SVRPKVPLRTMSYKLAIDMSHFIKEKGGLEGIYYSARRHRILDIYLEKEE |
| | Net | GIIPDWQDYTSGPGIRYPKTFGWLWKLVPVNVSDEAQEDEEHYLMHPAQT |
| | | SQWDDPWGEVLAWKFDPTLAYTYEAYVRYPEEFGSKSGLSEEEVKRRLTA |
| | | RGLLNMADKKETR |
| SEQ ID | clone 1.27 | FFREDLAFPQGKARKFSSEQTRANSPIRRERQVWRRDNNSLSEAGADRQG |
| NO: 20 | protein | TVSFNFPQITLWQRPLVTIKIGGQLKEALLDTGADDTVLEDMDLPGRWKP |
| | Pol | KMIGGIGGFIKVRQYDQIPIDICGHKAVGTVLVGPTPVNIIGRNLLTQIG |
| | | CTLNFPISPIETVPVKLKPGMDGPKVKQWPLTEEKIKALVEICTEMEKEG |
| | | KISKIGPENPYNTPVFAIKKKDSTKWRKLVDFRELNRKTQDFWEVQLGIP |
| | | HPAGLKKKKSVTVLDVGDAYFSVPLDKDFRKYTAFTIPSINNETPGIRYQ |
| | | YNVLPQGWKGSPAIFQSSMTKTLEPFRKQNPDIIIYQYMDDLYVGSDLEI |
| | | GQHRTKIEELRQHLLKWGFTTPDKKHQKEPPFLWMGYELHPDKWTVQPIV |
| | | LPEKDSWTVNDIQKLVGKLNWASQIYAGIKVKQLCKLLRGTKALTEVIPL |
| | | TKEAELELAENREILKEPVHGVYYDPSKDLIVEIQKQGQGQWTYQIFQEP |
| | | FKNLKTGKYAKTRGAHTNDVKQLTEAVQKIANESIVIWGKIPKFKLPIQK |
| | | ETWETWWTEYWQATWIPEWEFVNTPPLVKLWYQLEKEPIVGAETFYVDGA |
| | | ANRETKLGKAGYVTSRGRQKVVSLTDTTNQKTELQAIHLALQDSGLEVNI |
| | | VTDSQYALGIIQAQPDKSESELVSQIIEQLIKKEKVYLAWVPAHKGIGGN |
| | | EQVDKLVSAGIRRVLFLDGIEKAQEEHEKYHNNWRAMASEFNLPAVVAKE |
| | | IVACCDKCQVKGEAMHGQVDCSPGIWQLDCTHLEGKVILVAVHVASGYIE |
| | | AEVIPAETGQETAYFILKLAGRWPVKTIHTDNGSNFTSTTVKAACWWAGI |
| | | KQESGIPYNPQSQGVVESMNKELKKIIEQVRDQAEHLKTAVQMAVFIHNF |
| | | KRKGGIGGYSAGERIVDIIASDIQTKELQKQITKIQNFRVYYRDSRDPLW |
| | | KGPAKLLWKGEGAVVIQDKSDIKVVPRRKAKIIRDYGKQMAGDDCVASRQ |
| | | DED |
| SEQ ID | clone 1.27 | MGGAISMRRSRPSGNLYERLLRARGETYGKLLGEVKDGYSQSPGGLDKGL |
| NO: 21 | protein | SSLSCGGQKYNQGQYMNTPWRNPAKEREKLAYRKQNMDDIDKEDDDLVGV |
| | Nef | SVRPKVPLRTMSYKLAIDMSHFIKEKGGLEGIYYSARRHRILDIYLKKEE |
| | | GIIPDWQDYTSGPGIRYPKTFGWLWKLVPVNVSDEAQEDEEHYLMHPAQT |
| | | SQWDDPWGEVLAWKFDPTLAYTYEAYVRYPEEFGSKSGLSEEEVKRRLTA |
| | | RGLLNMADKKETR |
| SEQ ID | clone 1.10 | MGARASVLSGGKLDAWEKIRLRPGGKKKYRLKHLVWASRELERFALNPGL |
| NO: 22 | protein | LETSDGCKQIIGQLQPAIRTGSEELRSLFNTVATLYCVHERIEVKDTKEA |
| | Gag | LEKIEEEQNKSKKKAQQAAADTGHSNQVSQNYPIVQNIQGQMVHQALSPR |
| | | TLNAWVKVVEEKAFSPEVIPMFSALSEGATPQDLNTMLNTVGGHQAAMQM |
| | | LKETINEEAAEWDRVHPVHAGPIAPGQMREPRGSDIAGTTSTLQEQIGWM |
| | | THNPPIPVGEIYKRWIILGLNKIVRMYSPTSILDIRQGPKEPFRDYVDRF |
| | | YKTLRAEQATQEVKNWMTETLLVQNANPDCKTILKALGPAATLEEMMTAC |
| | | QGVGGPGHKARVLAEAMSQVTN.SATIMMQRGNFRNQRKTVKCFNCGKEG |
| | | HIAKNCRAPRKKGCWKCGKEGHQMKDCTERQANFLGKIWPSHKGRPGNFL |
| | | QSRPEPTAPSEESVRFGEETTTPSQKQEPIDKELYPLTSLRSLFGNDPSSQ |
| SEQ ID | clone 1.10 | FFREDLAFPQGKARKFSSEQTRANSPIRRERQVWRRDNNSLSEAGADRQG |
| NO: 23 | protein | TVSFNFPQITLWQRPLVTIKIGGQLKEALLDTGADDTVLEDMDLPGRWKP |
| | Pol | KMIGGIGGFIKVRQYDQIPIDICGHKAVGTVLVGPTPVNIIGRNLLTQIG |
| | | CTLNFPISPIETVPVKLKPGMDGPKVKQWPLTEEKIKALVEICTEMEKEG |
| | | KISKIGPENPYNTPVFAIKKKDSTKWRKLVDFRELNRKTQDFWEVQLGIP |
| | | HPAGLKKKKSVTVLDVGDAYFSVPLDKDFRKYTAFTIPSINNETPGIRYQ |
| | | YNVLPQGWKGSPAIFQSSMTKTLEPFRKQNPDIIIYQYMDDLYVGSDLEI |
| | | GQHRTKIEELRQHLLKWGFTTPDKKHQKEPPFLWMGYELHPDKWTVQPIV |
| | | LPEKDSWTVNDIQKLVGKLNWASQIYAGIKVKQLCKLLRGTKALTEVIPL |
| | | TKEAELELAENREILKEPVHGVYYDPSKDLIVEIQKQGQGQWTYQIFQEP |
| | | FKNLKTGKYAKTRSAHTNDVKQLTEAVQKIANESIVIWGKIPKFKLPIQK |
| | | ETWETWWTEYWQATWIPEWEFVNTPPLVKLWYQLEKEPIVGAETFYVDGA |
| | | ANRETKLGKAGYVTSRGRQKVVSLTDTTNQKTELQAIHLALQDSGLEVNI |
| | | VTDSQYALGIIQAQPDKSESELVSQIIEQLIKKEKVYLAWVPAHKGIGGN |
| | | EQVDKLVSAGIRRVLFLDGIEKAQEEHEKYHNNWRAMASEFNLPAVVAKE |
| | | IVACCDKCQVKGEAMHGQVDCSPGIWQLDCTHLEGKVILVAVHVASGYIE |
| | | AEVIPAETGQETAYFILKLAGRWPVKTIHTDNGSNFTSTTVKAACWWAGI |
| | | KQEFGIPYNPQSQGVVESMNKELKRIIEQVRDQAEHLKTAVQMAVFIHNF |
| | | KRKGGIGGYSAGERIVDIIASDIQTKELQKQITKIQNFRVYYRDSRDPLW |
| | | KGPAKLLWKGEGAVVIQDKSDIKVVPRRKAKIIRDYGKQMAGDDCVASRQ |
| | | DED |
| SEQ ID | clone 1.10 | MAGRSGKSDEDLLNTVRLIRLLYQSNPLPSLEGTRQARRNRRRRWRQRQR |
| NO: 24 | protein | QIQSISGWILSNHLGRPAEPVPLQLPPLERLTLDCNEDCGTSGTQGVGTP |
| | Rev | QILVEPPAVLGSGTKE |
| SEQ ID | clone 1.10 | MRVMGIRKNYQHLWKGGTLLLGILMICSAAEQLWVTVYYGVPVWKEANTT |
| NO: 25 | protein | LFCASDAKAYDTEVHNVWATHACVPTDPNPQEILLENVTEDFNMWKNNMV |
| | Env | EQMHEDIISLWDQSLKPCVKLTPLCVTLHCTDLKNGTNLKNGTNLKNGTK |
| | | IIGKSIRGEIKNCSFNVTKNIIDKVKKEYALFYRHDVVPIDRNITSYRLI |
| | | SCNTSTLTQACPKVSFEPIPIHYCAPAGFAILKCKDKKFNGTGPCTNVST |
| | | VQCTHGIRPVVSTQLLLNGSLAEEEVVIRSSNFTDNAKIIIVQLNETVEI |
| | | NCTRPNNNTRKGITLGPGRVFYTTGKIVGDIRKAHCNISKVKWHNTLKRV |
| | | VKKLREKFENKTIIFNKSSGGDPEIVMHSFNCGGEFFYCNTKKLFNSTWN |
| | | GTEGSYNIEGNDTITLPCRIKQIINMWQEVGKAMYAPPISGQIWCSSNIT |
| | | GLLLTRDGGKNSSTEIFRPGGGDIRDNWRSELYKYKVVRVEPLGIAPTKA |
| | | KRRVVQREKRAVGIGAVFLGFLGAAGSTMGAASITLTVQARQLLSGIVQQ |
| | | QNNLLRAIEAQQHMLQLTVWGIKQLQARVLAVERYLQDQQLLGIWGCSGK |
| | | LICTTTVPWNTSWSNKSLDTIWGNMTWMQWEKEINNYTGLIYNLIEESQN |
| | | QQEKNEQELLALDKWASLWNWFNISNWLWYIKIFIMIVGGLIGLRIVFSV |
| | | LSIVNRVRQGYSPLSFQTRFPASRGPDRPEGIEEEGGDRDRDRSSPLVDG |
| | | FLAIIWVDLRSLFLFSYHRLRDLLLIVTRIVELLGRRGWELLKYLWNLLQ |
| | | YWGQELKNSAVSLLNATAIAVGEGTDRIIEILQRAGRAILNIPTRIRQGL |
| | | ERALL |
| SEQ ID | clone 1.10 | MGGAISMRRSRPSGNLYERLLRARGETYGKLLGEVKDGYLQSPGGLDKGL |
| NO: 26 | protein | SSLSCEGQKYNQGQYMNTPWRNPAKEREKLAYRKQNMDDIDKEDDDLVGV |
| | Nef | SVRPKVPLRTMSYKVAIDMSHFIKEKGGLEGIYYSARRHRILDIYLEKEE |
| | | GIIPDWQDYTSGPGIRYPKTFGWLWKLVPVNVSDEAQEDEEHYLMHPAQT |
| | | SQWDDPWGEVLAWKFDPTLAYTYEAYVRYPEEFGSKSGLSEEEVKRRLTA |
| | | RGLLNMADKKETR |
| SEQ ID | clone | MGARASVLSGGKLDAWEKIRLRPGGKKKYRLKHLVWASRELERFALNPGL |
| NO: 27 | P10.21 | LETSDGCKQIIGQLQPAIRTGSEEFRSLFNTVATLYCVHERIEVKDTKEA |
| | protein | LEKIEEEQNKSKKKAQQAAADTGHSNQVSQNYPIVQNIQGQMVHQALSPR |
| | Gag | TLNAWVKVVEEKAFSPEVIPMFSALSEGATPQDLNTMLNTVGGHQAAMQM |
| | | LKETINEEAAEWDRVHPVHAGPIAPGQMREPRGSDIAGTTSTLQEQIGWM |
| | | THNPPIPVGEIYKRWIILGLNKIVRMYSPTSILDIRQGPKEPFRDYVDRF |
| | | YKTLRAEQATQEVKNWMTETLLVQNANPDCKTILKALGPAATLEEMMTAC |
| | | QGVGGPGHKARVLAEAMSQVTN.SATIMMQRGNFRNQRKTVKCFNCGKEG |
| | | HIAKNCRASRKKGCWKCGKEGHQMKDCTERQANFLGKIWPSHKGRPGNFL |
| | | QSRPEPTAPSEESVKFGEETTTPSQKQEPIDKELYPLTSLRSLFGNDPSSQ |
| SEQ ID | clone | MENRWQVMIVWQVDRMRIRTWKSLVKHHMYVSKKAKGWFYRHHYESTHPR |
| NO: 28 | P10.21 | ISSEVHIPLGDASLVVTTYWGLHTGERDWHLGQGVSIEWRKRRYSTQVDP |
| | protein | DLADQLTHLYYFDCFSESAIRNAILGHRVSPRCEYQAGHNKVGSLQYLAL |
| | Vif | AALVTPRKIKPPLPSVAKLTEDRWNKSHKTKGHRGSHTMNGH |
| SEQ ID | clone | MRVMGIRKNYQHLWKGGTLLLGILMICSAAEQLWVTVYYGVPVWKEANTT |
| NO: 29 | P10.21 | LFCASDAKAYDTEVHNVWATHACVPTDPNPQEILLENVTEDFNMWKNNMV |
| | protein | EQMHEDIISLWDQSLKPCVKLTPLCVTLHCTDLKNGTNLKNGTKIIGKSI |
| | Env | RGEIKNCSFNVTKNIIDKVKKEYALFYRHDVVPIDRNITSYRLISCNTST |
| | | LTQACPKVSFEPIPIHYCAPAGFAILKCKDKKFNGTGPCTNVSTVQCTHG |
| | | IRPVVSTQLLLNGSLAEEEVVIRSSNFTDNAKIIIVQLNETVEINCTRPN |
| | | NNTRKGITLGPGRVFYTTGKIVGDIRKAHCNISKVKWHNTLKRVVKKLRE |
| | | KFENKTIIFNKSSGGDPEIVMHSFNCGGEFFYCNTKKLFNSTWNGTEGSY |
| | | NIEGNDTITLPCRIKQIINMWQEVGKAMYAPPISGQIWCSSNITGLLLTR |
| | | DGGKNSSTEIFRPGGGDIRDNWRSELYKYKVVRVEPLGIAPTKAKRRVVQ |
| | | REKRAVGIGAVFLRFLGAAGSTMGAASITLTVQARQLLSGIVQQQNNLLR |
| | | AIEAQQHMLQLTVWGIKQLQARVLAVERYLQDQQLLGIWGCSGKLICTTT |
| | | VPWNTSWSNKSLDTIWGNMTWMQWEKEINNYTGLIYNLIEESQNQQEKNE |
| | | QELLALDKWASLWNWFNISNWLWYIKIFIMIVGGLIGLRIVFSVLSIVNR |
| | | VRQGYSPLSFQTRFPASRGPDRPEGIEEEGGDRDRDRSSPLVDGFLAIIW |
| | | VDLRSLFLFSYHRLRDLLLIVTRIVELLGRRGWELLKYLWNLLQYWSQEL |
| | | KNSAVSLLNATAIAVGEGTDRIIEILQRAGRAILNIPTRIRQGLERALL |
| SEQ ID | clone | MGGAISMRRSRPSGNLYERLLRARGETYGKLLGEVKDGYSQSPGGLDKGL |
| NO: 30 | P10.21 | SSLSCEGQKYNQGQYMNTPWRNPAKEREKLAYRKQNMDDIDKEDDDLVGV |
| | protein | SVRPKVPLRTMSYKLAIDMSHFIKEKGGLEGIYYSARRHRILDIYLEKEE |
| | Nef | GIIPDWQDYTSGPGIRYPKTFGWLWKLVPVNVSDEAQEDEEHYLTHPAQT |
| | | SQWDDPWGEVLAWKFDPTLAYTYEAYVRYPEEFGSKSGLSEEEVKRRLTA |
| | | RGLLNMADKKETR |
| SEQ ID | clone 1.26 | MENRWQVMIVWQVDRMRIRTWKSLVKHHMYVSKKAKGWFYRHHYKSTHPR |
| NO: 31 | protein | ISSEVHIPLGDASLVVTTYWGLHTGERDWHLGQGVSIEWRKRRYSTQVDP |
| | Vif | DLADQLIHLYYFDCFSESAIRNAILGHRVSPRCEYQAGHNKVGSLQYLAL |
| | | AALVTPRKIKPPLPSVAKLTEDRWNKSHKTKGHRGSHTMNGH |
| SEQ ID | clone 1.26 | MRVMGIRKNYQHLWKGGTLLLGILMICSAAEQLWVTVYYGVPVWKEANTT |
| NO: 32 | protein | LFCASDAKAYDTEVHNVWATHACVPTDPNPQEILLENVTEDFNMWKNNMV |
| | Env | EQMHEDIISLWDQSLKPCVKLTPLCVTLHCTDLKNGTNLKNGTKIIGKSI |
| | | RGEIKNCSFNVTKNIIDKVKKEYALFYRHDVVPIDRNITSYRLISCNTST |
| | | LTQACPKVSFEPIPIHYCAPAGFAILKCKDKKFNGTGPCTNVSTVQCTHG |
| | | IRPVVSTQLLLNGSLAEGEVVIRSSNFTDNAKIIIVQLNEAVEINCTRPN |
| | | NNTRKGITLGPGRVFYTTGKIVGDIRKAHCNISKVKWHNTLKRVVKKLRE |
| | | KFENKTIIFNKSSGGDPEIVMHSFNCGGEFFYCNTKKLFNSTWNGTEGSY |
| | | NIEGNDTITLPCRIKQIINMWQEVGKAMYAPPISGQIWCSSNITGLLLTR |
| | | DGGKNSSTEIFRPGGGDIRDNWRSELYKYKVVRVEPLGIAPTKAKRRVVQ |
| | | REKRAVGIGAVFLGFLGAAGSTMGAASITLTVQARQLLSGIVQQQNNLLR |
| | | AIEAQQHMLQLTVWGIKQLQARVLAVERYLQDQQLLGIWGCSGKLICTTT |
| | | VPWNTSWSNKSLDTIWGNMTWMQWEKEINNYTGLIYNLIEESQNQQEKNE |
| | | QELLALDKWASLWNWFNISNWLWYIKIFIMIVGGLIGLRIVFSVLSIVNR |
| | | VRQGYSPLSFQTRFPASRGPDRPEGIEEEGGDRDRDRSSPLVDGFLAIIW |
| | | VDLRSLFLFSYHRLRDLLLIVTRIVELLGRRGWELLKYLWNLLQYWSQEL |
| | | KNSAVSLLNATAIAVGEGTDRIIEILQRAGRAILNIPTRIRQGLERALL |
| SEQ ID | clone 1.26 | MGGAISMRRSRPSGNLYERLLRARGETYGKLLGEVKDGYSQSPGGLDKGL |
| NO: 33 | protein | SSLSCEGQKYNQGQYMNTPWRNPAKEREKLAYRKQNMDDINKEDDDLVGV |
| | Nef | SVRPKVPLRTMSYKLAIDMSHFIKEKGGLEGIYYSARRHRILDIYLEKEE |
| | | GIIPDWQDYTSGPGIRYPKTFGWLWKLVPVNVSDEAQEDEEHYLMHPAQT |
| | | SQWDDPWGEVLAWKFDPTLAYTYEAYVRYPEEFGSKSGLSEEEVKRRLTA |
| | | RGLLNMADKKETR |
| SEQ ID | clone | MGARASVLSGGKLDAWEKIRLRPGGKKKYRLKHLVWASRELERFALNPGL |
| NO: 34 | P8A26 | LETSDGCKQIIGQLQPAIRTGSEELRSLFNTVATLYCVHERIEVKDTKEA |
| | protein | LEKIEEEQNKSKKKAQQAAADTGHSNQVSQNYPIVQNIQGQMVHQALSPR |
| | Gag | TLNAWVKVVEEKAFSPEVIPMFSALSEGATPQDLNTMLNTVGGHQAAMQM |
| | | LKETINEEAAEWDRVHPVHAGPIAPGQMREPRGSDIAGTTSTLQEQIGWM |
| | | THNPPIPVGEIYKRWIILGLNKIVRMYSPTSILDIRQGPKEPFRDYVDRF |
| | | YKTLRAEQATQEVKNWMTETLLVQNANPDCKTILKALGPAATLEEMMTAC |
| | | QGVGGPGHKARVLAEAMSQVTNSATIMMQRGKFRNQRKTVKCFNCGKEGH |
| | | IAKNCRAPRKKGCWKCGKEGHQMKDCTERQANFLGKIWPSHKGRPGNFLQ |
| | | SRPEPTAPSEESVRFGEETTTPSQKQEPIDKELYPLTSLRSLFGNDPSSQ |
| SEQ ID | clone | FFREDLAFPQGKARKFSSEQTRANSPIRRERQVWRGDNNSLSEAGADRQG |
| NO: 35 | P8A26 | TVSFNFPQITLWQRPLVTIKIGGQLKEALLDTGADDTVLEDMDLPGRWKP |
| | protein | KMIGGIGGFIKVRQYDQIPIDICGHKAVGTVLVGPTPVNIIGRNLLTQIG |
| | Pol | CTLNFPISPIETVPVKLKPGMDGPKVKQWPLTEEKIKALVEICTEMEKEG |
| | | KISKIGPENPYNTPVFAIKKKDSTKWRKLVDFRELNRKTQDFWEVQLGIP |
| | | HPAGLKKKKSVTVLDVGDAYFSVPLDKDFRKYTAFTIPSINNETPGIRYQ |
| | | YNVLPQGWKGSPAIFQSSMTKTLEPFRKQNPDIIIYQYMDDLYVGSDLEI |
| | | GQHRTKIEELRQHLLKWGFTTPDKKHQKEPPFLWMGYELHPDKWTVQPIV |
| | | LPEKDSWTVNDIQKLVGKLNWASQIYAGIKVKQLCKLLRGTKALTEVIPL |
| | | TKEAELELAENREILKEPVHGVYYDPSKDLIVEIQKQGQGQWTYQIFQEP |
| | | FKNLKTGKYAKTRGAHTNDVKQLTEAVQKIANESIVIWGKIPKFKLPIQK |
| | | ETWETWWTEYWQATWIPEWEFVNTPPLVKLWYQLEKEPIVGAETFYVDGA |
| | | ANRETKLGKAGYVTSRGRQKVVSLTDTTNQKTELQAIHLALQDSGLEVNI |
| | | VTDSQYALGIIQAQPDKSESELVSQIIEQLIKKEKVYLTWIPAHKGIGGN |
| | | EQVDKLVSAGIRRVLFLDGIEKAQEEHEKYHSNWRAMASEFNLPAVVAKE |
| | | IVACCDKCQVKGEAMHGQVDCSPGIWQLDCTHLEGKVILVAVHVASGYIE |
| | | AEVIPAETGQETAYFILKLAGRWPVKTIHTDNGSNFTSTTVKAACWWAGI |
| | | KQEFGIPYNPQSQGVVESMNKELKKIIEQVRDQAEHLKTAVQMAVFIHNF |
| | | KRKGGIGGYSAGERIVDIIASDIQTKELQKQITKIQNFRVYYRDSRDPLW |
| | | KGPAKLLWKGEGAVVIQDKSDIKVVPRRKAKIIRDYGKQMAGDDCVASRQ |
| | | DED |
| SEQ ID | clone | MEQVPQDQGPQREPYNEWTLELLEELKNEAVRHFPRPWLHGLGQYIYETY |
| NO: 36 | P8A26 | EDTWAGVEAIIRILQQLLLIHFRIGCQHSRIGIIRQRRTRNGASRS |
| | protein | |
| | Vpr | |
| SEQ ID | clone | MQPLVILAIVALVVALIIVIVVWSIVLIEYRKILRQKKIDRLIDRIRERA |
| NO: 37 | P8A26 | EDSGNESDGDQEELSALVERGHLAPWNIDDL |
| | protein | |
| | Vpu | |
| SEQ ID | clone | MRVMGIRKNYQHLWKGGTLLLGILMICSAAEQLWVTVYYGVPVWKEANTT |
| NO: 38 | P8A26 | LFCASDAKAYDTEVHNVWATHACVPTDPNPQEILLENVTEDFNMWKNNMV |
| | protein | EQMHEDIISLWDQSLKPCVKLTPLCVTLHCTDLKNGTNLKNGTKIIGKSM |
| | Env | RGEIKNCSFNVTKNIIDKVKKEYALFYRHDVVPIDRNITSYRLISCNTST |
| | | LTQACPKVSFEPIPIHYCAPAGFAILKCKDKKFNGTGPCTNVSTVQCTHG |
| | | IRPVVSTQLLLNGSLAEEEVVIRSSNFTDNAKIIIVQLNETV*INCTRPN |
| | | NNTRKGITLGPGRVFYTTGKIVGDIRKAHCNISKVKWHNTLKRVVEKLRE |
| | | KFENKTIIFNKSSGGDPEIVMHSFNCGGEFFYCNTKKLFNSTWNGTEGSY |
| | | NIEGNDTITLPCRIKQIINMWQEVGKAMYAPPISGQIWCSSNITGLLLTR |
| | | DGGKNSSTEIFRPGGGDIRDNWRSELYKYKVVRVEPLGIAPTKAKRRVVQ |
| | | REKRAVGIGAVFLGFLGAAGSTMGAASITLTVQARQLLSGIVQQQNNLLR |
| | | AIEAQQHMLQLTVWGIKQLQARVLAVERYLQDQQLLGIWGCSGKLICTTT |
| | | VPWNTSWSNKSLDTIWGNMTWMQWEKEINNYTGLIYNLIEESQNQQEKNE |
| | | QELLALDKWASLWNWFNISNWLWYIKIFIMIVGGLIGLRIVFSVLSIVNR |
| | | VRQGYSPLSFQTRFPASRGPDRPEGIEEEGGDRDRDRSSPLVDGFLAIIW |
| | | VDLRSLFLFSYHRLRDLLLIVTRIVELLGRRGWELLKYLWNLLQYWSQEL |
| | | KNSTVSLLNATAIAVGEGTDRIIEILQRAGRAILNIPTRIRQGLERALL |
| SEQ ID | clone | MGGAISMRRSRPSGDLYERLLRARGETYGRLLGEVEDGYSQSPGGLDKGL |
| NO: 39 | P8A26 | SSLSCEGQKYNQGQYMNTPWRNPAKEKEKLAYRKQNMNDINKEDDNLVGV |
| | protein | SVRPKVPLRTMSYKLAIDMSHFIKEKGGLEGIYYSARRHRILDIYLEKEE |
| | Nef | GIIPDWQDYTSGPGIRYPKTFGWLWKLVPVNVSDEAQEDEEHYLMHPAQT |
| | | SQWDDPWGEVLAWKFDPTLAYTYEAYVKYPEEFGSKSGLSEEEVRRRLTA |
| | | RGLLNMADKKETR |
| SEQ ID | parent | TGGAAGGGCTAATTTACTCCCAGAAAAGACAAGATATCCTTGACCTGTGG |
| NO: 40 | DH12 DNA | GTTTACAACACACAAGGCTACTTCCCTGACTGGCAGAACTACACACCAGG |
| | (GenBank | GCCAGGAATCAGATATCCCCTGACCTTTGGGTGGTGCTTCAAGCTAGTAC |
| | Accession | CAGTAGATCCAGAGAAGGTAGAAGCGGCCAATGAAGGAGAGAACAACTGC |
| | No.: | TTGTTACACCCTATAAGCCTGCATGGAATGGAGGACCCGGAGAAAGAAGT |
| | AF069140) | GTTGCTGTGGAAGTTTGACAGTCGCCTAGCATATCATCACATGGCCCGAG |
| | | AGCTGCATCCGGAGTACTACAAGAACTGCTGACACCGAGCTATCTACAGG |
| | | GGACTTTCCGCTGGGGACTTTCCAGGGAGGCGTGGCCTGGGCGGGACCGG |
| | | GGAGTGGCGAGCCCTCAGATGCTGCATATAAGCAGCCGCTTTTGCCTGTA |
| | | CTGGGTCTCTCTAGTTAGACCAGATCTGAGCCTGGGAGCTCTCTGGCTAG |
| | | CTGAGAACCCACTGCTTAAGCCTCAATAAAGCTTGCCTTGAGTGCTTTAA |
| | | GTAGTGTGTGCCCGTCTGTTGTGTGACTCTGGTAACTAGAGATCCCTCAG |
| | | ACCATTTTAGTCAGTGTGGAAAATCTCTAGCAGTGGCGCCCGAACAGGGA |
| | | CCGGAAAGCGAAAGAGAAACCAGAGAAGCTCTCTCGACGCAGGACTCGGC |
| | | TTGCTGAAGCGCGCACGGCAAGAGGCGAGGGGCGGCGAACGGTGAGTACG |
| | | CCAAAATTTTGACTAGCGGAGGCTAGAAGGAGAGAGATGGGTGCGAGAGC |
| | | GTCAGTATTAAGCGGCGGAAAATTAGATAGTTGGGAAAAAATTCGATTAA |
| | | GGCCAGGGGGAAAGAAAAAATATAAATTAAAACATATAGTATGGGCAAGC |
| | | AGGGAGCTAGAACGGTTCGCAGTCAATCCTGGCCTGTTAGAAACATCAGA |
| | | AGGCTGCAGACAAATACTGGGACAGCTACAACCGTCCCTTCAGACAGGAT |
| | | CAGAAGAACTTAGATCACTATATAATACAGTAGCAACCCTCTATTGTGTG |
| | | CATGAAAGGATAGAGGTAAAAGACACCAAGGAAGCTTTAGACAAGGTAGA |
| | | GGAAGAGCAAAACAAAAGTAAGAAAAAAGCACAGCAAGCAGCAGCTGACA |
| | | CAGGAAACAGCAGTCAAGTCAGCCAAAATTACCCTATAGTGCAGAACATT |
| | | CAGGGGCAAATGGTACATCAGGCCCTATCACCTAGAACTTTAAATGCGTG |
| | | GGTAAAAGTAGTAGAAGAGAAGGCTTTTAGCCCAGAAGTAATACCCATGT |
| | | TTTCAGCATTATCAGAAGGAGCCACCCCACAAGATTTAAACACCATGCTA |
| | | AACACAGTGGGGGGACATCAGGCAGCCATGCAAATGTTAAAAGAGACTAT |
| | | CAATGAGGAAGCTGCAGAATGGGATAGATTGCATCCAGTGCATGCAGGGC |
| | | CTATTGCACCAGGCCAGATGAGAGAACCAAGGGGAAGTGACATAGCAGGA |
| | | ACTACTAGTACCCTGCAGGAACAAATAGGATGGATGACAAACAATCCACC |
| | | TATCCCAGTAGGAGAAATTTATAAAAGATGGATAATCATGGGATTAAATA |
| | | AAATAGTAAGGATGTACAGTCCTACCAGCATTCTGGATATAAGACAAGGA |
| | | CCAAAGGAACCCTTTAGAGATTATGTAGACCGGTTCTATAAAACTCTAAG |
| | | AGCCGAGCAAGCTTCACAGGAAGTAAAAAATTGGATGACAGAAACCTTGT |
| | | TGGTCCAAAATTCGAACCCAGATTGTAAGACTATTTTGAAAGCATTGGGA |
| | | CCAGGAGCTACACTAGAAGAAATGATGACAGCATGTCAGGGAGTAGGAGG |
| | | ACCTGGCCATAAAGCAAGAGTTTTGGCTGAAGCAATGAGCCAGATAACAA |
| | | ATACTTCAGCTACCATAATGATGCAGGGAGGCAATTTTAGGAACCAAAGA |
| | | AAGATTAAGTGTTTCAATTGTGGCAAAGAAGGGCACATATCCAAAAATTG |
| | | CAGGGCCCCTAGGAAAAAGGGCTGTTGGAAATGTGGAAAGGAAGGACATC |
| | | AAATGAAAGATTGTACTGAGAGACAGGCTAATTTTTTAGGGAAAATCTGG |
| | | CCTTCCCACAAGGAAAGGCCAGGGAATTTTCTTCAGAGCAGACCAGAGCC |
| | | ATCAGCCCCACCAGAAGAGAGCTTCAGGTTTGGGGAGGAGACAGCAACTC |
| | | CCTCTCAGAAGCAGGAGCCGAAGGAACTATATCCCTTAGCCTCCCTCAAA |
| | | TCACTCTTTGGCAACGACCCCTAGTCAAGATAAAAATAGGGGGGCAACTA |
| | | AAAGAAGCTCTATTAGATACAGGAGCAGATGATACAGTATTAGAAGAAAT |
| | | AAATTTGCCAGGAAAATGGAAACCAAAAATGATAGGGGGAATTGGAGGTT |
| | | TTATCAAAGTAAGACAGTATGATCAGGTACTCATAGAAATTTGTGGACAT |
| | | AAAGCTATAGGTACAGTATTAGTAGGACCTACACCTGTCAACATAATTGG |
| | | AAGAAATCTGTTGACTCAGATTGGTTGCACTTTAAATTTTCCCATTAGTC |
| | | CTATTGAAACTGTACCAGTAAAATTAAAGCCAGGAATGGATGGCCCAAGA |
| | | GTTAAACAATGGCCATTGTCAGAAGAGAAAATAAAAGCATTAACAGAAAT |
| | | TTGTACAGAAATGGAAAAGGAAGGGAAAATTTCAAAAATTGGGCCTGAAA |
| | | ATCCATACAATACTCCAATATTTGCCATAAAGAAAAAGAACAGTACTAGA |
| | | TGGAGAAAATTAGTAGATTTCAGAGAACTTAATAAGAGAACTCAAGACTT |
| | | CTGGGAAGTTCAATTAGGAATACCGCATCCCGCAGGGTTAAAAAAGAAAA |
| | | AGTCAGTAACAGTACTGGACGTGGGTGATGCATATTTTTCAATTCCCTTA |
| | | GATGAAGACTTTAGGAAGTATACTGCATTTACCATACCTAGTGTAAACAA |
| | | TGCAGCACCAGGGATTAGATATCAGTACAATGTGCTTCCACAGGGATGGA |
| | | AAGGATCACCAGCAATATTCCAAAGTAGCATGACAAAAATCTTAGAACCT |
| | | TTTAGAAAACAAAATCCAGACATAGTAATCTATCAATACATGGATGATTT |
| | | GTATGTAGGATCTGACTTAGAAATAGAACAGCATAGAACAAAAATAGAGG |
| | | AACTGAGACAACATCTGTTGAGGTGGGGACTTTTCACACCAGACCAAAAA |
| | | CATCAGAAAGAACCTCCATTCCTTTGGATGGGTTATGAACTCCATCCTGA |
| | | TAAGTGGACAGTACAGCCTATAGTGCTGCCAGAAAAGGACAGCTGGACTG |
| | | TCAATGACATACAGAAGTTAGTGGGAAAATTAAATTGGGCAAGTCAGATT |
| | | TACGCAGGGATTAAAGTAAAGCAATTATGTAAACTCCTTAGAGGAGCTAA |
| | | AGCACTAACAGAAGTAATACCACTAACAGAAGAAGCAGAGTTAGAACTGG |
| | | CAGAAAACAGGGAGATTCTAAAAGAACCAGTACATGGAGTGTATTATGAC |
| | | CCATCAAAAGACATAATAGCAGAGATACAGAAACAGGGGCAAGGCCAATG |
| | | GACATATCAAATTTATCAGGAACCATTTAAAAATCTGAAAACAGGAAAAT |
| | | ATGCAAGAACGAGGGGTGCCCACACTAATGATGTAAAACAATTAACAGAG |
| | | GTAGTGCAAAAAGTAACCACAGAGTGCATAGTAATATGGGGAAAGACTCC |
| | | TAAATTTAGACTACCCATACAAAAAGAAACATGGGAAACATGGTGGACAG |
| | | AGTATTGGCAAGCCACCTGGATTCCTGAGTGGGAGTTTGTCAATACCCCT |
| | | CCCTTAGTAAAATTATGGTACCAGTTAGAGAAAGAACCCATAGTAGGGGC |
| | | AGAAACTTTCTATGTAGATGGGGCAGCTAGCAGGGAAACTAGATTAGGAA |
| | | AGGCAGGATATGTTACTAACAGAGGAAGACAAAAGGTTGTCTCCCTAACT |
| | | GACACAACAAATCAGAAGACTGAGTTACAAGCAATTTATCTAGCTTTGCA |
| | | GGATTCGGGATTAGAAGTAAACATAGTAACAGACTCACAATATGCATTAG |
| | | GAATCATTCAAGCACAACCAGATAAAAGTGAATCAGAGTTAGTCAATCAA |
| | | ATAATAGAGCAGTTAATAAAAAAGGAAAAGGTCTACCTGGCATGGGTACC |
| | | AGCACACAAAGGAATTGGAGGAAATGAACAAGTAGATAAATTAGTCAGTA |
| | | CTGGAATCAGGAGAGTACTATTTCTAGATGGAATAGAGAAGGCCCAAGAA |
| | | GAACATGAGAAATATCATAGTAATTGGAGAGCAATGGCTAGTGAATTTAA |
| | | CCTGCCAGCTGTAGTAGCAAAAGAGATAGTAGCCTGCTGTGATAAGTGCC |
| | | AGGTAAAAGGAGAAGCCATGCATGGACAAGTAGACTGCAGTCCAGGAATA |
| | | TGGCAACTAGATTGTACACATTTAGAAGGAAAAGTTATCCTGGTAGCAGT |
| | | TCATGTAGCCAGTGGATATATAGAAGCAGAGGTTATTCCAGCAGAGACAG |
| | | GACAGGAAACAGCATACTTTATTTTAAAATTAGCAGGAAGATGGCCAGTA |
| | | AAAACAATACATACAGACAATGGCAGTAATTTCACCAGTACTACGGTTAA |
| | | GGCCGCCTGTTGGTGGGCAGGGATCAAGCAGGAATTTGGCATTCCCTACA |
| | | ATCCCCAAAGTCAAGGAGTAGTAGAATCTATGAATAAAGAATTAAAGAAA |
| | | ATTATAGAACAAGTAAGAGATCAGGCTGAACATCTTAAGACAGCAGTACA |
| | | AATGGCAGTATTCATTCACAATTTTAAAAGAAAAGGGGGGATTGGGGGGT |
| | | ACAGTGCAGGGGAAAGAATAGTAGACATAATAGCATCAGACATACAAACT |
| | | AAAGAACTACAAAAACAAATTACAAAAATTCAAAATTTTCGGGTTTATTA |
| | | CAGGGACAGCAGAGATCCACTTTGGAAAGGACCAGCAAAGCTTCTTTGGA |
| | | AAGGTGAAGGGGCAGTAGTAATACAAGATAAGAGTGACATAAAAGTAGTG |
| | | CCAAGAAGAAAAGCAAAGATTATCAGGGATTATGGAAAACAGATGGCAGG |
| | | TGATGATTGTGTGGCAAGTAGACAGGATGAGGATTAGAACATGGAAAAGT |
| | | TTAGTAAAACACCATATGTATGTTTCAAAGAAAGCTAAGGGATGGTTTTA |
| | | TAGACATCACTATGAAAGCACTCATCCAAGAATAAGTTCAGAAGTACATA |
| | | TCCCACTAGGGGATGCTAGCTTGGTAGTAACAACATATTGGGGTCTACAT |
| | | ACAGGAGAAAGAGACTGGCATTTGGGTCAGGGAGTCTCCATAGAATGGAG |
| | | GAAAAGGAGATACAGCACACAAGTAGACCCTGACCTAGCAGACCAACTAA |
| | | TTCATCTGTACTACTTTGATTGTTTTTCAGAATCTGCTATAAGAAATGCC |
| | | ATATTAGGACATAGAGTTAGTCCTAGGTGTGAATATCAAGCAGGACATAA |
| | | CAAGGTAGGATCTCTACAGTACTTGGCACTAGCAGCATTAGTAACACCAA |
| | | GAAAGATAAAGCCACCTTTGCCTAGTGTTGCGAAACTGACAGAGGACAGA |
| | | TGGAACAAGTCCCACAAGACCAAGGGCCACAGAGGGAGCCATACAATGAA |
| | | TGGACACTAGAGCTTTTAGAGGAGCTTAAGAATGAAGCTGTCAGACATTT |
| | | CCCTAGACCATGGCTTCATGGCCTAGGGCAATATATCTATGAAACTTATG |
| | | GGGATACTTGGGCAGGAGTGGAAGCCATAATAAGAATTCTGCAACAATTG |
| | | CTGCTTATTCATTTCAGAATTGGGTGTCAACATAGCAGAATAGGCATTAT |
| | | TCGACAGAGGAGAACAAGAAATGGAGCCAGTAGATCCTAGACTAGAGCCC |
| | | TGGAAGCATCCAGGAAGTCAGCCTAAGACTGCCTGTACCAATTGCTATTG |
| | | CAAAAAGTGTTGCTTGCATTGCCAAGTTTGCTTCATAACAAAAGGCTTAG |
| | | GCATCTCCTATGGCAGGAAGAAGCGGAGAAAGCGACGAAGATCTCCTCAA |
| | | CACAGTCAGACTGATCAAGCTTCTCTATCAAAGCAGTAAGTAGTACATGT |
| | | AATGCAACCTTTAGTAATATTAGCAATAGTAGCATTAGTAGTAGCACTAA |
| | | TAATAGTCATAGTTGTATGGTCCATTGTATTAATAGAATATAGAAAAATA |
| | | TTAAGACAAAAGAAAATAGACAGGTTAATTGATAGAATAAGAGAAAGAGC |
| | | AGAAGACAGTGGCAATGAGAGTGATGGGGATCAGGAAGAATTATCAGCAC |
| | | TTGTGGAAAGGGGGCACCTTGCTCCTTGGGATATTGATGATCTGTAGTGC |
| | | TGCAGAACAATTGTGGGTCACAGTCTATTATGGGGTACCTGTGTGGAAAG |
| | | AAGCAAACACCACTCTATTTTGTGCATCAGATGCTAAAGCATATGATACA |
| | | GAGGTACATAATGTTTGGGCCACACATGCCTGTGTACCCACAGACCCCAA |
| | | CCCACAAGAAATACTATTGGAAAATGTGACAGAAGATTTTAACATGTGGA |
| | | AAAATAACATGGTAGAACAGATGCATGAGGATATAATCAGTTTATGGGAT |
| | | CAAAGTCTAAAGCCATGTGTAAAATTAACCCCACTCTGTGTTACTTTACA |
| | | TTGCACTGATTTGAAGAATGGTACTAATTTGAAGAATGGTACTAAAATCA |
| | | TTGGGAAATCAATGAGAGGAGAAATAAAAAACTGCTCTTTCAATGTCACC |
| | | AAAAACATAATAGATAAGGTGAAAAAAGAATATGCGCTTTTCTATAGACA |
| | | TGATGTAGTACCAATAGATAGGAATATTACTAGCTATAGGTTGATAAGTT |
| | | GTAACACCTCAACCCTTACACAGGCCTGTCCAAAGGTATCCTTTGAGCCA |
| | | ATTCCCATACATTATTGTGCCCCGGCTGGTTTTGCGATTCTAAAATGTAA |
| | | AGATAAGAAGTTCAATGGAACGGGACCATGTACAAATGTCAGTACAGTAC |
| | | AATGTACACATGGAATTAGGCCAGTAGTATCAACTCAACTGCTGTTAAAT |
| | | GGAAGTCTAGCAGAAGAAGAGGTAGTAATTAGATCTAGCAATTTCACGGA |
| | | CAATGCTAAAATCATAATAGTACAGCTGAATGAAACTGTAGAAATTAATT |
| | | GTACAAGACCCAACAACAATACAAGAAAAGGGATAACTCTAGGACCAGGG |
| | | AGAGTATTTTATACAACAGGAGAAATAGTAGGAGATATAAGAAAAGCACA |
| | | TTGTAACATTAGTAAAGTAAAATGGCATAACACTTTAAAAAGGGTAGTTG |
| | | AAAAATTAAGAGAAAAATTTGAAAATAAAACAATAGTCTTTAATAAATCC |
| | | TCAGGGGGGGACCCAGAAATTGTAATGCACAGCTTTAATTGTGGAGGGGA |
| | | ATTTTTCTACTGTAATACAAAAAAACTGTTTAATAGTACTTGGAATGGTA |
| | | CTGAAGGGTCATATAACATTGAAGGAAATGACACTATCACACTCCCATGC |
| | | AGAATAAAACAAATTATAAACATGTGGCAGGAAGTAGGAAAAGCAATGTA |
| | | TGCCCCTCCCATCAGTGGACAAATTTGGTGCTCATCAAATATTACAGGGC |
| | | TGCTACTAACAAGAGATGGTGGTAAGAACAGCAGCACCGAAATCTTCAGA |
| | | CCTGGAGGAGGAGATATGAGGGACAATTGGAGAAGTGAATTATATAAATA |
| | | TAAAGTAGTAAGAGTTGAACCATTAGGAATAGCACCCACCAAGGCAAAGA |
| | | GAAGAGTGGTGCAGAGAGAAAAAAGAGCAGTGGGAATAGGAGCTGTGTTC |
| | | CTTGGGTTCTTGGGAGCAGCAGGAAGCACTATGGGCGCAGCGTCAATAAC |
| | | GCTGACGGTACAGGCCAGACAATTATTGTCTGGTATAGTGCAACAGCAGA |
| | | ACAATTTGCTGAGGGCTATTGAAGCGCAACAGCATATGTTGCAACTCACA |
| | | GTCTGGGGCATCAAGCAGCTCCAGGCAAGAGTCCTGGCTGTGGAAAGATA |
| | | CCTACAGGATCAACAGCTCCTGGGGATTTGGGGTTGCTCTGGAAAACTCA |
| | | TCTGCACCACTACTGTGCCTTGGAATACTAGTTGGAGTAATAAATCTCTG |
| | | GATACAATTTGGGGTAACATGACCTGGATGCAGTGGGAAAAAGAAATTAA |
| | | CAATTACACAGGCTTAATATACAACTTGATTGAAGAATCGCAGAACCAAC |
| | | AAGAAAAGAATGAACAAGAATTATTGGCATTAGATAAATGGGCAAGTTTG |
| | | TGGAATTGGTTTAACATATCAAACTGGCTGTGGTATATAAAAATATTCAT |
| | | AATGATAGTAGGAGGCTTGATAGGTTTAAGAATAGTTTTCAGTGTACTTT |
| | | CTATAGTGAATAGAGTTAGGCAGGGATACTCACCATTATCGTTTCAGACC |
| | | CGCTTCCCAGCCTCGAGGGGACCCGACAGGCCCGAAGGAATCGAAGAAGA |
| | | AGGTGGAGACAGAGACAGAGACAGATCCAGTCCATTAGTGGATGGATTCT |
| | | TAGCAATCATCTGGGTCGACCTGCGGACGCTGTTCCTCTTCAGCTACCAC |
| | | CGCTTGAGAGACTTACTCTTGATTGTAACGAGGATTGTGGAACTTCTGGG |
| | | ACGCAGGGGGTGGGAACTCCTCAAATACTTGTGGAATCTCCTGCAGTATT |
| | | GGAGTCAGGAACTAAAGAATAGTGCTGTTAGCTTGCTTAACGCCACAGCC |
| | | ATAGCAGTAGGTGAGGGAACAGATAGGATTATAGAAATATTACAAAGAGC |
| | | TGGTAGAGCTATTCTCAACATACCTACGAGAATAAGACAGGGCTTAGAAA |
| | | GGGTTTGCTATAAGATGGGTGGCAAGTTGTCAAAGTGTGGTGGGGTGGG |
| | | ATGGTCTACTGTAAGGGAAAGAATGAGACGAGCTGAGCCAGCAGCAGATC |
| | | GTGAGCCAGCAGTAGGGGTGGGAGCAGCATCTCGAGACCTGGGAAAACAT |
| | | GGAGCAATCACAAGTAGCAATACAGCAGCTACCAATGCTGATTGTGCCTG |
| | | GTTAGAAGCACAACAAGAGGAGGAGGAGGTGGGTTTTCCAGTCAGACCTC |
| | | AGATACCTTTAAGACCAATGACCTATAAGGCAGCTTTAGATCTTAGCCAC |
| | | TTTTTAAAAGAAAAGGGGGGACTGGAAGGGCTAATTTACTCCCAGAAAAG |
| | | ACAAGATATCCTTGACCTGTGGGTTTACAACACACAAGGCTACTTCCCTG |
| | | ACTGGCAGAACTACACACCAGGGCCAGGAATCAGATATCCCCTGACCTTT |
| | | GGGTGGTGCTTCAAGCTAGTACCAGTAGATCCAGAGAAGGTAGAAGCGGC |
| | | CAATGAAGGAGAGAACAACTGCTTGTTACACCCTATAAGCCTGCATGGAA |
| | | TGGAGGACCCGGAGAAAGAAGTGTTGCTGTGGAAGTTTGACAGTCGCCTA |
| | | GCATATCATCACATGGCCCGAGAGCTGCATCCGGAGTACTACAAGAACTG |
| | | CTGACACCGAGCTATCTACAGGGGACTTTCCGCTGGGGACTTTCCAGGGA |
| | | GGCGTGGCCTGGGCGGGACCGGGGAGTGGCGAGCCCTCAGATGCTGCATA |
| | | TAAGCAGCCGCTTTTGCCTGTACTGGGTCTCTCTAGTTAGACCAGATCTG |
| | | AGCCTGGGAGCTCTCTGGCTAGCTGAGAACCCACTGCTTAAGCCTCAATA |
| | | AAGCTTGCCTTGAGTGCTTTAAGTAGTGTGTGCCCGTCTGTTGTGTGACT |
| | | CTGGTAACTAGAGATCCCTCAGACCATTTTAGTCAGTGTGGAAAATCTCT |
| | | AGCA |
| SEQ ID | parent | GenBank Accession No. K03455, M38432 |
| NO: 41 | HXB2 DNA | |
| SEQ ID | parent LAI | GenBank Accession No. K02013 |
| NO: 42 | DNA | |
| SEQ ID | parent | GenBank Accession No. M19921 |
| NO: 43 | NL4-3 DNA | |
| SEQ ID | parent AD- | GenBank Accession No. AF004394 |
| NO: 44 | 8 DNA | |
| SEQ ID | parent YU- | GenBank Accession No. M93258 |
| NO: 45 | 2 DNA | |
| SEQ ID | parent | GenBank Accession No. M38429 |
| NO: 46 | JRCSF DNA | |
| SEQ ID | parent | GenBank Accession No. M22639 |
| NO: 47 | Z2Z6 DNA | |
| SEQ ID | parent ELI | GenBank Accession No. K03454, X04414 |
| NO: 48 | DNA | |
| SEQ ID | parent MAL | GenBank Accession No. X04415, K03456 |
| NO: 49 | DNA | |
| SEQ ID | clone 1.4 | UGGAAGGGAUUUAUUACAGUGCAAGAAGACAUAGAAUCUUAGACAUAUAC |
| NO: 50 | RNA | UUAAAAAAGGAAGAAGGCAUCAUACCAGAUUGGCAGGAUUACACCUCAGG |
| | | ACCAGGAAUUAGAUACCCAAAGACAUUUGGCUGGCUAUGGAAAUUAGUCC |
| | | CUGUAAAUGUAUCAGAUGAGGCACAGGAGGAUGAGGAGCAUUAUUUAAUG |
| | | CAUCCAGCUCAAACUUCCCAGUGGGAUGACCCUUGGGGAGAGGUUCUAGC |
| | | AUGGAAGUUUGAUCCAACUCUGGCCUACACUUAUGAGGCAUAUGUUAGAU |
| | | ACCCAGAAGAGUUUGGAAGCAAGUCAGGCCUGUCAGAGGAAGAGGUUAAA |
| | | AGAAGGCUAACCGCAAGAGGCCUUCUUAACAUGGCUGACAAGAAGGAAAC |
| | | UCGCUGAAUUCGAGCUAUCUACAGGGGACUUUCCGCUGGGGACUUUCCAG |
| | | GGAGGCGUGGCCUGGGCGGGACCGGGGAGUGGCGAGCCCUCAGAUGCUGC |
| | | AUAUAAGCAGCCGCUUUUGCCUGUACUGGGUCUCUCUAGUUAGACCAGAU |
| | | CUGAGCCUGGGAGCUCUCUGGCUAGCUGAGAACCCACUGCUUAAGCCUCA |
| | | AUAAAGCUUGCCUUGAGUGCUUUAAGUAGUGUGUGCCCGUCUGUUGUGUG |
| | | ACUCUGGUAACUAGAGAUCCCUCAGACCAUUUUAGUCAGUGUGGAAAAUC |
| | | UCUAGCAGUGGCGCCCGAACAGGGACCGGAAAGCGAAAGAGAAACCAGAG |
| | | AAGCUCUCUCGACGCAGGACUCGGCUUGCUGAAGCGCGCACGGCAAGCGG |
| | | CGAGGGGCAGCGACCGGUGAGUACGCUAAAAAUUUUGACUAGCGGAGGCU |
| | | AGAAGGAGAGAGAUGGGUGCGAGAGCGUCAGUAUUAAGCGGGGGAAAAUU |
| | | GGAUGCAUGGGAAAAAAUUCGGUUACGGCCAGGAGGAAAGAAAAAAUAUA |
| | | GACUAAAACAUCUAGUAUGGGCAAGCAGGGAGCUAGAACGAUUUGCACUU |
| | | AAUCCUGGCCUUUUAGAGACAUCAGAUGGCUGUAAACAAAUAAUAGGACA |
| | | GCUACAACCAGCUAUCCGGACAGGAUCAGAAGAACUUAGAUCAUUAUUUA |
| | | AUACAGUAGCAACCCUCUAUUGUGUACAUGAAAGGAUAGAGGUAAAAGAC |
| | | ACCAAGGAAGCUUUAGAGAAGAUAGAGGAAGAGCAAAACAAAAGUAAGAA |
| | | AAAAGCACAGCAAGCAGCAGCUGACACAGGACACAGCAAUCAGGUCAGCC |
| | | AAAAUUACCCUAUAGUGCAGAACAUCCAGGGGCAAAUGGUACAUCAGGCC |
| | | CUAUCACCUAGAACUUUAAAUGCGUGGGUAAAAGUAGUAGAAGAGAAGGC |
| | | UUUUAGCCCAGAAGUAAUACCCAUGUUUUCAGCAUUAUCAGAAGGAGCCA |
| | | CCCCACAAGAUUUAAACACCAUGCUAAACACAGUGGGGGGACAUCAAGCA |
| | | GCCAUGCAAAUGUUAAAAGAGACCAUCAAUGAGGAAGCUGCAGAAUGGGA |
| | | UAGAGUGCAUCCAGUGCAUGCAGGGCCUAUUGCACCAGGCCAGAUGAGAG |
| | | AACCAAGGGGAAGUGACAUAGCAGGAACUACUAGUACCCUUCAGGAACAA |
| | | AUAGGAUGGAUGACACAUAAUCCACCUAUCCCAGUAGGAGAAAUCUAUAA |
| | | AAGAUGGAUAAUCCUGGGAUUAAAUAAAAUAGUAAGAAUGUAUAGCCCUA |
| | | CCAGCAUUCUGGACAUAAGACAAGGACCAAAGGAACCCUUUAGAGACUAU |
| | | GUAGACCGGUUCUAUAAAACCCUAAGAGCCGAGCAAGCUACACAGGAGGU |
| | | AAAAAAUUGGAUGACAGAAACCUUGUUGGUCCAAAAUGCGAACCCAGAUU |
| | | GUAAAACUAUUUUAAAAGCAUUGGGACCAGCAGCCACACUAGAAGAAAUG |
| | | AUGACAGCAUGUCAGGGAGUGGGGGGACCCGGCCAUAAAGCAAGAGUUUU |
| | | GGCUGAAGCAAUGAGCCAAGUAACAAAUUCAGCUACCAUAAUGAUGCAGA |
| | | GAGGCAAUUUUAGGAACCAAAGAAAAACUGUUAAGUGUUUCAAUUGUGGC |
| | | AAAGAAGGGCACAUAGCCAAAAAUUGCAGGGCUCCUAGGAAAAAGGGCUG |
| | | UUGGAAAUGUGGAAAGGAAGGACACCAAAUGAAAGAUUGUACUGAGAGAC |
| | | AGGCUAAUUUUUUAGGGAAGAUCUGGCCUUCCCACAAGGGAAGGCCAGGA |
| | | AAUUUUCUUCAGAGCAGACCAGAGCCAACAGCCCCAUCAGAAGAGAGCGU |
| | | CAAGUUUGGAGAAGAGACAACAACUCCCUCUCAGAAGCAGGAGCCGAUAG |
| | | ACAAGGAACUGUAUCCUUUAACUUCCCUCAGAUCACUCUUUGGCAACGAC |
| | | CCCUCGUCACAAUAAAGAUAGGGGGGCAACUAAAGGAAGCUCUAUUAGAU |
| | | ACAGGAGCAGAUGAUACAGUAUUAGAAGACAUGGAUUUGCCAGGAAGAUG |
| | | GAAACCAAAAAUGAUAGGGGGAAUUGGAGGUUUUAUCAAAGUAAGACAGU |
| | | AUGAUCAGAUACCCAUAGAUAUCUGUGGACAUAAAGCUGUAGGUACAGUA |
| | | UUAGUAGGACCUACACCUGUCAACAUAAUUGGAAGAAAUCUGUUGACUCA |
| | | GAUUGGUUGCACUUUAAAUUUUCCCAUUAGUCCUAUUGAAACUGUACCAG |
| | | UAAAAUUAAAGCCAGGAAUGGAUGGCCCAAAAGUCAAACAAUGGCCAUUG |
| | | ACAGAAGAAAAAAUAAAAGCAUUAGUAGAAAUUUGUACAGAAAUGGAAAA |
| | | GGAAGGAAAGAUUUCAAAAAUUGGGCCUGAAAAUCCAUACAAUACUCCAG |
| | | UAUUUGCCAUAAAGAAAAAAGACAGUACUAAAUGGAGAAAAUUAGUAGAU |
| | | UUCAGAGAACUUAAUAGGAAAACUCAAGACUUCUGGGAAGUUCAAUUAGG |
| | | AAUACCACAUCCCGCAGGGUUAAAAAAGAAAAAAUCAGUAACAGUACUGG |
| | | AUGUGGGUGAUGCAUAUUUUUCAGUUCCCUUAGAUAAAGACUUCAGGAAG |
| | | UAUACUGCAUUUACCAUACCUAGUAUAAACAAUGAGACACCAGGGAUUAG |
| | | AUAUCAGUACAAUGUGCUUCCACAGGGAUGGAAAGGAUCACCAGCAAUAU |
| | | UCCAAAGUAGCAUGACAAAAACCUUAGAGCCUUUUAGAAAACAAAAUCCA |
| | | GACAUAAUUAUCUAUCAAUACAUGGAUGAUUUGUAUGUAGGAUCUGACUU |
| | | AGAAAUAGGGCAGCAUAGAACAAAAAUAGAGGAACUGAGACAACAUCUGU |
| | | UGAAGUGGGGAUUUACCACACCAGACAAAAAACAUCAGAAAGAACCUCCA |
| | | UUCCUUUGGAUGGGUUAUGAACUCCAUCCUGAUAAAUGGACAGUACAGCC |
| | | UAUAGUGCUGCCAGAAAAAGACAGCUGGACUGUCAAUGACAUACAGAAGU |
| | | UAGUGGGAAAAUUAAAUUGGGCAAGUCAAAUUUAUGCAGGGAUUAAAGUA |
| | | AAGCAAUUAUGUAAACUCCUUAGGGGAACCAAAGCACUUACAGAAGUAAU |
| | | ACCACUAACAAAAGAAGCAGAGCUAGAACUGGCAGAAAACAGGGAGAUUC |
| | | UAAAGGAACCAGUACAUGGAGUGUAUUAUGACCCAUCAAAAGACUUAAUA |
| | | GUAGAAAUACAGAAGCAGGGGCAAGGCCAAUGGACAUAUCAAAUUUUUCA |
| | | AGAGCCAUUUAAAAAUCUGAAAACAGGAAAAUAUGCAAAAACGAGGGGUG |
| | | CCCACACUAAUGAUGUAAAACAAUUAACAGAGGCAGUGCAAAAAAUAGCC |
| | | AAUGAAAGCAUAGUAAUAUGGGGAAAGAUUCCUAAAUUUAAAUUACCCAU |
| | | ACAAAAAGAAACAUGGGAAACAUGGUGGACAGAGUAUUGGCAAGCCACCU |
| | | GGAUUCCUGAGUGGGAGUUUGUCAAUACCCCUCCCUUAGUGAAAUUAUGG |
| | | UACCAGUUAGAAAAAGAACCCAUAGUAGGAGCAGAAACUUUCUAUGUAGA |
| | | UGGGGCAGCUAACAGGGAGACUAAAUUAGGAAAAGCAGGAUAUGUUACUA |
| | | GCAGAGGAAGGCAAAAAGUUGUCUCCCUAACAGACACAACAAAUCAGAAA |
| | | ACUGAGUUACAAGCAAUUCACCUAGCUUUGCAGGAUUCAGGAUUAGAAGU |
| | | AAACAUAGUAACAGACUCACAAUAUGCAUUAGGAAUCAUUCAAGCACAAC |
| | | CAGAUAAAAGUGAAUCAGAGUUAGUCAGUCAAAUAAUAGAACAGCUAAUA |
| | | AAAAAGGAAAAAGUCUACCUGGCAUGGGUACCAGCACACAAAGGAAUUGG |
| | | AGGAAAUGAACAGGUAGAUAAAUUAGUCAGUGCUGGAAUCAGGAGAGUAC |
| | | UAUUUCUAGAUGGAAUAGAGAAGGCCCAAGAAGAACAUGAGAAAUAUCAU |
| | | AAUAAUUGGAGAGCAAUGGCUAGUGAAUUUAACCUGCCAGCUGUAGUAGC |
| | | AAAAGAGAUAGUAGCCUGCUGUGAUAAGUGCCAGGUAAAAGGAGAAGCCA |
| | | UGCAUGGACAAGUAGACUGCAGUCCAGGAAUAUGGCAACUAGAUUGUACA |
| | | CAUUUAGAAGGAAAAGUUAUCCUGGUAGCAGUUCAUGUAGCCAGUGGAUA |
| | | UAUAGAAGCAGAGGUUAUUCCAGCAGAGACAGGACAGGAAACAGCAUACU |
| | | UUAUUUUAAAAUUAGCAGGAAGAUGGCCAGUAAAAACAAUACAUACAGAC |
| | | AAUGGCAGUAAUUUCACCAGUACUACGGUUAAGGCCGCCUGUUGGUGGGC |
| | | AGGGAUCAAGCAGGAAUUUGGCAUUCCCUACAAUCCCCAAAGUCAAGGAG |
| | | UAGUAGAAUCUAUGAAUAAAGAAUUAAAGAAAAUUAUAGAACAAGUAAGA |
| | | GAUCAGGCUGAACAUCUUAAGACAGCAGUACAAAUGGCAGUAUUCAUUCA |
| | | CAAUUUUAAAAGAAAAGGGGGGAUUGGGGGGUACAGUGCAGGGGAAAGAA |
| | | UAGUAGACAUAAUAGCAUCAGACAUACAAACUAAAGAACUACAAAAACAA |
| | | AUCACAAAAAUUCAAAAUUUUCGGGUUUAUUACAGGGACAGCAGAGAUCC |
| | | ACUUUGGAAAGGACCAGCAAAGCUUCUUUGGAAAGGUGAAGGGGCAGUAG |
| | | UAAUACAAGAUAAGAGUGACAUAAAAGUAGUGCCAAGAAGAAAAGCAAAG |
| | | AUUAUCAGGGAUUAUGGAAAACAGAUGGCAGGUGAUGAUUGUGUGGCAAG |
| | | UAGACAGGAUGAGGAUUAGAACAUGGAAAAGUUUAGUAAAACACCAUAUG |
| | | UAUGUUUCAAAGAAAGCUAAGGGAUGGUUUUAUAGACAUCACUAUGAAAG |
| | | CACUCAUCCAAGAAUAAGUUCAGAAGUACAUAUCCCACUAGGGGAUGCUA |
| | | GCUUGGUAGUAACAACAUAUUGGGGUCUACAUACAGGAGAAAGAGACUGG |
| | | CAUUUGGGUCAGGGAGUCUCCAUAGAAUGGAGGAAAAGGAGAUACAGCAC |
| | | ACAAGUAGACCCUGACCUAGCAGACCAACUAAUUCAUCUGUACUACUUUG |
| | | AUUGUUUUUCAGAAUCUGCUAUAAGAAAUGCCAUAUUAGGACAUAGAGUU |
| | | AGUCCUAGGUGUGAAUAUCAAGCAGGACAUAACAAGGUAGGAUCUCUACA |
| | | GUACUUGGCACUAGCAGCAUUAGUAACACCAAGAAAGAUAAAGCCACCUU |
| | | UGCCUAGUGUUGCGAAACUGACAGAGGACAGAUGGAACAAGUCCCACAAG |
| | | ACCAAGGGCCACAGAGGGAGCCAUACAAUGAAUGGACACUAAAGCUUUUA |
| | | GAGGAGCUUAAGAAUGAAGCUGUCAGACAUUUCCCUAGACCAUGGCUUCA |
| | | UGGCCUAGGGCAAUAUAUCUAUGAAACUUAUGAGGAUACUUGGGCAGGAG |
| | | UGGAAGCCAUAAUAAGAAUUCUGCAACAAUUGCUGCUUAUUCAUUUCAGA |
| | | AUUGGGUGUCAACAUAGCAGAAUAGGCAUUAUUCGACAGAGGAGAACAAG |
| | | AAAUGGAGCCAGUAGAUCCUAGACUAGAGCCCUGGAAGCAUCCAGGAAGU |
| | | CAGCCUAAGACUGCCUGUACCAAUUGCUAUUGCAAAAAGUGUUGCUUGCA |
| | | UUGCCAAGUUUGCUUCAUAACAAAAGGCUUAGGCAUCUCCUAUGGCAGGA |
| | | AGAAGCGGAAAAAGCGACGAAGAUCUCCUCAACACAGUCAGACUGAUCAA |
| | | GCUUCUCUAUCAAAGCAGUAAGUAGUACAUGUAAUGCAACCUUUAGUAAU |
| | | AUUAGCAAUAGUAGCAUUAGUAGUAGCACUAAUAAUAGUCAUAGUUGUAU |
| | | GGUCCAUUGUAUUAAUAGAAUAUAGAAAAAUAUUAAGACAAAAGAAAAUA |
| | | GACAGGUUAAUUGAUAGAAUAAGAGAAAAAGCAGAAGACAGUGGCAAUGA |
| | | GAGUGAUGGGGAUCAGGAAGAAUUAUCAGCACUUGUGGAAAGGGGGCACC |
| | | UUGCUCCUUGGGAUAUUGAUGAUCUGUAGUGCUGCAGAACAAUUGUGGGU |
| | | CACAGUCUAUUAUGGGGUACCUGUGUGGAAAGAAGCAAACACCACUCUAU |
| | | UUUGUGCAUCAGAUGCUAAGGCAUAUGAUACAGAGGUACAUAAUGUUUGG |
| | | GCCACACAUGCCUGUGUACCCACAGACCCCAACCCACAAGAAAUACUAUU |
| | | GGAAAAUGUGACAGAAGAUUUUAACAUGUGGAAAAAUAACAUGGUAGAAC |
| | | AGAUGCAUGAGGAUAUAAUCAGUUUAUGGGAUCAAAGUCUAAAGCCAUGU |
| | | GUAAAAUUAACCCCACUCUGUGUUACUUUACAUUGCACUGAUUUGAAGAA |
| | | UGGUACUAAUUUGAAGAAUGGUACUAAAAUCAUUGGGAAAUCAAUAAGAG |
| | | GAGAAAUAAAAAACUGCUCUUUCAAUGUCACCAAAAACAUAAUAGAUAAG |
| | | GUGAAAAAAGAAUAUGCGCUUUUCUAUAGACAUGAUGUAGUACCAAUAGA |
| | | UAGGAAUAUUACUAGCUAUAGGUUAAUAAGUUGUAACACCUCAACCCUUA |
| | | CACAGGCCUGUCCAAAGGUAUCCUUUGAGCCAAUUCCCAUACAUUAUUGU |
| | | GCCCCGGCUGGUUUUGCGAUUCUAAAAUGUAAAGAUAAGAAGUUCAAUGG |
| | | AACGGGACCAUGUACAAAUGUCAGUACAGUACAAUGUACACAUGGAAUUA |
| | | GGCCAGUAGUAUCAACUCAACUGCUGUUAAAUGGAAGUCUAGCAGAAGAA |
| | | GAGGUAGUAAUUAGAUCUAGCAAUUUCACGGACAAUGCUAAAAUCAUAAU |
| | | AGUACAGCUGAAUGAAACUGUAGAAAUUAAUUGUACAAGACCCAACAACA |
| | | AUACAAGAAAAGGGAUAACUCUAGGACCAGGGAGAGUAUUUUAUACAACA |
| | | GGAAAAAUAGUAGGAGAUAUAAGAAAAGCACAUUGUAACAUUAGUAAAGU |
| | | AAAAUGGCAUAACACUUUAAAAAGGGUAGUUAAAAAAUUAAGAGAAAAAU |
| | | UUGAAAAUAAAACAAUAAUCUUUAAUAAAUCCUCAGGGGGGGACCCAGAA |
| | | AUUGUAAUGCACAGCUUUAAUUGUGGAGGGGAAUUUUUCUACUGUAAUAC |
| | | AAAAAAACUGUUUAAUAGUACUUGGAAUGGUACUGAAGGGUCAUAUAACA |
| | | UUGAAGGAAAUGACACUAUCACACUCCCAUGCAGAAUAAAACAAAUUAUA |
| | | AACAUGUGGCAGGAAGUAGGAAAAGCAAUGUAUGCCCCUCCCAUCAGUGG |
| | | ACAAAUUUGGUGCUCAUCAAAUAUUACAGGGCUGCUACUAACAAGAGAUG |
| | | GUGGUAAGAACAGCAGCACCGAAAUCUUCAGACCUGGAGGAGGAGAUAUA |
| | | AGGGACAAUUGGAGAAGUGAAUUAUAUAAAUAUAAAGUAGUAAGAGUUGA |
| | | ACCAUUAGGAAUAGCACCCACCAAGGCAAAAAGAAGAGUGGUGCAGAGAG |
| | | AAAAAAGAGCAGUGGGAAUAGGAGCUGUGUUCCUUGGGUUCUUGGGAGCA |
| | | GCAGGAAGCACUAUGGGCGCAGCGUCAAUAACGCUGACGGUACAGGCCAG |
| | | ACAAUUAUUGUCUGGUAUAGUGCAACAGCAGAACAAUUUGCUGAGGGCUA |
| | | UUGAAGCGCAACAGCAUAUGUUGCAACUCACAGUCUGGGGCAUCAAGCAG |
| | | CUCCAGGCAAGAGUCCUGGCUGUGGAAAGAUACCUACAGGAUCAACAGCU |
| | | CCUGGGGAUUUGGGGUUGCUCUGGAAAACUCAUCUGCACCACUACUGUGC |
| | | CUUGGAAUACUAGUUGGAGUAAUAAAUCUCUGGAUACAAUUUGGGGUAAC |
| | | AUGACCUGGAUGCAGUGGGAAAAAGAAAUUAACAAUUACACAGGCUUAAU |
| | | AUACAACUUGAUUGAAGAAUCGCAGAACCAACAAGAAAAGAAUGAACAAG |
| | | AAUUAUUGGCAUUAGAUAAAUGGGCAAGUUUGUGGAAUUGGUUUAACAUA |
| | | UCAAACUGGCUGUGGUAUAUAAAAAUAUUCAUAAUGAUAGUAGGAGGCUU |
| | | GAUAGGUUUAAGAAUAGUUUUCAGUGUACUUUCUAUAGUGAAUAGAGUUA |
| | | GGCAGGGAUACUCACCAUUAUCGUUUCAGACCCGCUUCCCAGCCUCGAGG |
| | | GGACCCGACAGGCCCGAAGGAAUCGAAGAAGAAGGUGGAGACAGAGACAG |
| | | AGACAGAUCCAGUCCAUUAGUGGAUGGAUUCUUAGCAAUCAUCUGGGUCG |
| | | ACCUGCGGAGCCUGUUCCUCUUCAGCUACCACCGCUUGAGAGACUUACUC |
| | | UUGAUUGUAACGAGGAUUGUGGAACUUCUGGGACGCAGGGGGUGGGAACU |
| | | CCUCAAAUACUUGUGGAAUCUCCUGCAGUAUUGGAGUCAGGAACUAAAGA |
| | | AUAGUGCUGUUAGCUUGCUUAACGCCACAGCCAUAGCAGUAGGUGAGGGA |
| | | ACAGAUAGAAUUAUAGAAAUAUUACAAAGAGCUGGUAGAGCUAUUCUCAA |
| | | CAUACCUACGAGAAUAAGACAGGGCUUAGAAAGGGCUUUGCUAUAAGCUU |
| | | AUGGGUGGAGCUAUUUCCAUGAGGCGGUCCAGGCCGUCUGGAAAUCUGUA |
| | | CGAGAGACUCUUGCGGGCGCGUGGGGAGACUUAUGGAAAACUCUUAGGAG |
| | | AGGUAAAAGAUGGAUACUCGCAAUCCCCAGGAGGAUUAGACAAGGGCUUG |
| | | AGCUCACUCUCUUGUGAGGGACAAAAAUACAAUCAGGGACAGUAUAUGAA |
| | | UACUCCAUGGAGAAACCCAGCUAAAGAGAGAGAAAAAUUAGCAUACAGAA |
| | | AACAAAAUAUGGAUGAUAUAGAUAAGGAAGAUGAUGACUUGGUAGGGGUA |
| | | UCAGUGAGGCCAAAAGUUCCCCUAAGAACAAUGAGUUACAAAUUGGCAAU |
| | | AGACAUGUCUCAUUUUAUAAAAGAAAAGGGGGGACUGGAAGGGAUUUAUU |
| | | ACAGUGCAAGAAGACAUAGAAUCUUAGACAUAUACUUAAAAAAGGAAGAA |
| | | GGCAUCAUACCAGAUUGGCACGAUUACACCUCAGGACCAGGAAUUAGAUA |
| | | CCCAAAGACAUUUGGCUGGCUAUGGAAAUUAGUCCCUGUAAAUGUAUCAG |
| | | AUGAGGCACAGGAGGAUGAGGAGCAUUAUUUAAUGCAUCCAGCUCAAACU |
| | | UCCCAGUGGGAUGACCCUUGGGGAGAGGUUCUAGCAUGGAAGUUUGAUCC |
| | | AACUCUGGCCUACACUUAUGAGGCAUAUGUUAGAUACCCAGAAGAGUUUG |
| | | GAAGCAAGUCAGGCCUGUCAGAGGAAGAGGUUAAAAGAAGGCUAACCGCA |
| | | AGAGGCCUUCUUAACAUGGCUGACAAGAAGGAAACUCGCUGAAUUCGAGC |
| | | UAUCUACAGGGGACUUUCCGCUGGGGACUUUCCAGGGAGGCGUGGCCGCU |
| | | GCGGGACCGGGGAGUGGCGAGCCCUCAGAUGCUGCAUAUAAGCAGCCGCU |
| | | UUUGCCUGUACUGGGUCUCUCUAGUUAGACCAGAUCUGAGCCUGGGAGCU |
| | | CUCUGGCUAGCUGAGAACCCACUGCUUAAGCCUCAAUAAAGCUUGCCUUG |
| | | AGUGCUUUAAGUAGUGUGUGCCCGUCUGUUGUGUGACUCUGGUAACUAGA |
| | | GAUCCCUCAGACCAUUUUAGUCAGUGUGGAAAAUCUCUAGCA |
| SEQ ID | clone | UGGAAGGGAUUUAUUACAGUGCAAGAAGACAUAGAAUCUUAGACAUAUAC |
| NO: 51 | P10.26 RNA | UUAGAAAAGGAAGAAGGCAUCAUACCAGAUUGGCAGGAUUACACCUCAGG |
| | | ACCAGGAAUUAGAUACCCAAAGACAUUUGGCUGGCUAUGGAAAUUAGUCC |
| | | CUGUAAAUGUAUCAGAUGAGGCACAGGAGGAUGAGGAGCAUUAUUUAAUG |
| | | CAUCCAGCUCAAACUUCCCAGUGGGAUGACCCUUGGGGAGAGGUUCUAGC |
| | | AUGGAAGUUUGAUCCAACUCUGGCCUACACUUAUGAGGCAUAUGUUCUGAU |
| | | ACCCAGAAGAGUUUGGAAGCAAGUCAGGCCUGUCAGAGGAAGAGGUUAAA |
| | | AGAAGGCUAACCGCAAGAGGCCUUCUUAACAUGGCUGACAAGAAGGAAAC |
| | | UCGCUGAAUUCGAGCUAUCUACAGGGGACUUUCCGCUGGGGACUUUCCAG |
| | | GGAGGCGUGGCCUGGGCGGGACCGGGGAGUGGCGAGCCCUCAGAUGCUGC |
| | | AUAUAAGCAGCCGCUUUUGCCUGUACUGGGUCUCUCUAGUUAGACCAGAU |
| | | CUGAGCCUGGGAGCUCUCUGGCUAGCUGAGAACCCACUGCUUAAGCCUCA |
| | | AUAAAGCUUGCCUUGAGUGCUUUAAGUAGUGUGUGCCCGUCUGUUGUGUG |
| | | ACUCUGGUAACUAGAGAUCCCUCAGACCAUUUUAGUCAGUGUGGAAAAUC |
| | | UCUAGCAGUGGCGCCCGAACAGGGACCGGAAAGCGAAAGAGAAACCAGAG |
| | | AAGCUCUCUCGACGCAGGACUCGGCUUGCUGAAGCGCGCACGGCAAGCGG |
| | | CGAGGGGCAGCGACCGGUGAGUACGCUAAAAAUUUUGACUAGCGGAGGCU |
| | | AGAAGGAGAGAGAUGGGUGCGAGAGCGUCAGUAUUAAGCGGGGGAAAAUU |
| | | GGAUGCAUGGGAAAAAAUUCGGUUACGGCCAGGAGGAAAGAAAAAAUAUA |
| | | GACUAAAACAUCUAGUAUGGGCAAGCAGGGAGCUAGAACGAUUUGCACUU |
| | | AAUCCUGGCCUUUUAGAGACAUCAGAUGGCUGUAAACAAAUAAUAGGACA |
| | | GCUACAACCAGCUAUCCGGACAGGAUCAGAAGAACUUAGAUCAUUAUUUA |
| | | AUACAGUAGCAACCCUCUAUUGUGUACAUGAAAGGAUAAAGGUAAAAGAC |
| | | ACCAAGGAAGCUUUAGAGAAGAUAGAGGAAGAGCAAAACAAAAGUAAGAA |
| | | AAAAGCACAGCAAGCAGCAGCUGACACAGGACACAGCAAUCAGGUCAGCC |
| | | AAAAUUACCCUAUAGUGCAGAACAUCCAGGGGCAAAUGGUACAUCAGGCC |
| | | CUAUCACCUAGAACUUUAAAUGCGUGGGUAAAAGUAGUAGAAGAGAAGGC |
| | | UUUUAGCCCAGAAGUAAUACCCAUGUUUUCAGCAUUAUCAGAAGGAGCCA |
| | | CCCCACAAGAUUUAAACACCAUGCUAAACACAGUGGGGGGACAUCAAGCA |
| | | GCCAUGCAAAUGUUAAAAGAGACCAUCAAUGAGGAAGCUGCAGAAUGGGA |
| | | UAGAGUGCAUCCAGUGCAUGCAGGGCCUAUUGCACCAGGCCAGAUGAGAG |
| | | AACCAAGGGGAAGUGACAUAGCAGGAACUACUAGUACCCUUCAGGAACAA |
| | | AUAGGAUGGAUGACACAUAAUCCACCUAUCCCAGUAGGAGAAAUCUAUAA |
| | | AAGAUGGAUAAUCCUGGGAUUAAAUAAAAUAGUAAGAAUGUAUAGCCCUA |
| | | CCAGCAUUCUGGACAUAAGACAAGGACCAAAGGAACCCUUUAGAGACUAU |
| | | GUAGACCGGUUCUAUAAAACCCUAAGAGCCGAGCAAGCUACACAGGAGGU |
| | | AAAAAAUUGGAUGACAGAAACCUUGUUGGUCCAAAAUGCGAACCCAGAUU |
| | | GUAAAACUAUUUUAAAAGCAUUGGGACCAGCAGCCACACUAGAAGAAAUG |
| | | AUGACAGCAUGUCAGGGAGUGGGAGGACCCGGCCAUAAAGCAAGAGUUUU |
| | | GGCUGAAGCAAUGAGCCAAGUAACAAAUUCAGCUACCAUAAUGAUGCAGA |
| | | GAGGCAAUUUUAGGAACCAAAGAAAAACUGUUAGGUGUUUCAAUUGUGGC |
| | | AAAGAAGGGCACAUAGCCAAAAAUUGCAGGGCUCCUAGGAAAAAGGGCUG |
| | | UUGGAAAUGUGGAAAGGAAGGACACCAAAUGAAAGAUUGUACUGAGAGAC |
| | | AGGCUAAUUUUUUAGGGAAGAUCUGGCCUUCCCACAAGGGAAGGCCAGGA |
| | | AAUUUUCCUCAGAGCAGACCAGAGCCAACAGCCCCAUCAGAAGAGAGCGU |
| | | CAAGUUUGGAGAAGAGACAACAACUCCCUCUCAGAAGCAGGAGCCGAUAG |
| | | ACAAGGAACUGUAUCCUUUAACUUCCCUCAGAUCACUCUUUGGCAACGAC |
| | | CCCUCGUCACAAUAAAGAUAGGGGGGCAACUAAAGGAAGCUCUAUUAGAU |
| | | ACAGGAGCAGAUGAUACAGUAUUAGAAGACAUGGAUUUGCCAGGAAGAUG |
| | | GAAACCAAAAAUGAUAGGGGGAAUUGGAGGUUUUAUCAAAGUAAGACAGU |
| | | AUGAUCAGAUACCCAUAGAUAUCUGUGGACAUAAAGCUGUAGGUACAGUA |
| | | UUAGUAGGACCUACACCUGUCAACAUAAUUGGAAGAAAUCUGUUGACUCA |
| | | GAUUGGUUGCACUUUAAAUUUUCCCAUUAGUCCUAUUGAAACUGUACCAG |
| | | UAAAAUUAAAGCCAGGAAUGGAUGGCCCAAAAGUCAAACAAUGGCCAUUG |
| | | ACAGAAGAAAAAAUAAAAGCAUUAGUAGAAAUUUGUACAGAAAUGGAAAA |
| | | GGAAGGAAAGAUUUCAAAAAUUGGGCCUGAAAAUCCAUACAAUACUCCAG |
| | | UAUUUGCCAUAAAGAAAAAAGACAGUACUAAAUGGAGAAAAUUAGUAGAU |
| | | UUCAGAGAACUUAAUAGGAAAACUCAAGACUUCUGGGAAGUUCAAUUAGG |
| | | AAUACCACAUCCCGCAGGGUUAAAAAAGAAAAAAUCAGUAACAGUACUGG |
| | | AUGUGGGUGAUGCAUAUUUUUCAGUUCCCUUAGAUAAAGACUUCAGGAAG |
| | | UAUACUGCAUUUACCAUACCUAGUAUAAACAAUGAGACACCAGGGAUUAG |
| | | AUAUCAGUACAAUGUGCUUCCACAGGGAUGGAAAGGAUCACCAGCAAUAU |
| | | UCCAAAGUAGCAUGACAAAAACCUUAGAGCCUUUUAGAAAACAAAAUCCA |
| | | GACAUAAUUAUCUAUCAAUACAUGGAUGAUUUGUAUGUAGGAUCUGACUU |
| | | AGAAAUAGGGCAGCAUAGAACAAAAAUAGAGGAACUGAGACAACAUCUGU |
| | | UGAAGUGGGGAUUUACCACACCAGACAAAAAACAUCAGAAAGAACCUCCA |
| | | UUCCUUUGGAUGGGUUAUGAACUCCAUCCUGAUAAAUGGACAGUACAGCC |
| | | UAUAGUGCUGCCAGAAAAAGACAGCUGGACUGUCAAUGACAUACAGAAGU |
| | | UAGUGGGAAAAUUGAAUUGGGCAAGUCAAAUUUAUGCAGGGAUUAAAGUA |
| | | AAGCAAUUAUGUAAACUCCUUAGGGGAACCAAAGCACUUACAGAAGUAAU |
| | | ACCACUAACAAAAGAAGCAGAGCUAGAACUGGCAGAAAACAGGGAGAUUC |
| | | UAAAGGAACCAGUACAUGGAGUGUAUUAUGACCCAUCAAAAGACUUAAUA |
| | | GUAGAAAUACAGAAGCAGGGGCAAGGCCAAUGGACAUAUCAAAUUUUUCA |
| | | AGAGCCAUUUAAAAAUCUGAAAACAGGAAAAUAUGCAAAAACGAGGGGUG |
| | | CCCACACUAAUGAUGUAAAACAAUUAACAGAGGCAGUGCAAAAAAUAGCC |
| | | AAUGAAAGCAUAGUAAUAUGGGGAAAGAUUCCUAAAUUUAAAUUACCCAU |
| | | ACAAAAAGAAACAUGGGAAACAUGGUGGACAGAGUAUUGGCAAGCCACCU |
| | | GGAUUCCUGAGUGGGAGUUUGUCAAUACCCCUCCCUUAGUGAAAUUAUGG |
| | | UACCAGUUAGAAAAAGAACCCAUAGUAGGAGCAGAAACUUUCUAUGUAGA |
| | | UGGGGCAGCUAACAGGGAGACUAAAUUAGGAAAAGCAGGAUAUGUUACUA |
| | | GCAGAGGAAGGCAAAAAGUUGUCUCCCUAACAGACACAACAAAUCAGAAA |
| | | ACUGAGUUACAAGCAAUUCACCUAGCUUUGCAGGAUUCAGGAUUAGAAGU |
| | | AAACAUAGUAACAGACUCACAAUAUGCAUUAGGAAUCAUUCAAGCACAAC |
| | | CAGAUAAAAGUGAAUCAGAGUUAGUCAGUCAAAUAAUAGAACAGCUAAUA |
| | | AAAAAGGAAAAAGUCUACCUGGCAUGGGUACCAGCACACAAAGGAAUUGG |
| | | AGGAAAUGAACAGGUAGAUAAAUUAGUCAGUGCUGGAAUCAGGAGAGUAC |
| | | UAUUUCUAGAUGGAAUAGAGAAGGCCCAAGAAGAACAUGAGAAAUAUCAU |
| | | AAUAAUUGGAGAGCAAUGGCUAGUGAAUUUAACCUGCCAGCUGUAGUAGC |
| | | AAAAGAGAUAGUAGCCUGCUGUGAUAAGUGCCAGGUAAAAGGAGAAGCCA |
| | | UGCAUGGACAAGUAGACUGCAGUCCAGGAAUAUGGCAACUAGAUUGUACA |
| | | CAUUUAGAAGGAAAAGUUAUCCUGGUAGCAGUUCAUGUAGCCAGUGGAUA |
| | | UAUAGAAGCAGAGGUUAUUCCAGCAGAGACAGGACAGGAAACAGCAUACU |
| | | UUAUUUUAAAAUUAGCAGGAAGAUGGCCAGUAAAAACAAUACAUACAGAC |
| | | AAUGGCAGUAAUUUCACCAGUACUACGGUUAAGGCCGCCUGUUGGUGGGC |
| | | AGGGAUCAAGCAGGAAUUUGGCAUUCCCUACAAUCCCCAAAGUCAAGGAG |
| | | UAGUAGAAUCUAUGAAUAAAGAAUUAAAGAAAAUUAUAGAACAAGUAAGA |
| | | GAUCAGGCUGAACAUCUUAAGACAGCAGUACAAAUGGCAGUAUUCAUUCA |
| | | CAAUUUUAAAAGAAAAGGGGGGAUUGGGGGGUACAGUGCAGGGGAAAGAA |
| | | UAGUAGACAUAAUAGCAUCAGACAUACAAACUAAAGAACUACAAAAACAA |
| | | AUUACAAAAAUUCAAAAUUUUCGGGUUUAUUACAGGGACAGCAGAGAUCC |
| | | ACUUUGGAAAGGACCAGCAAAGCUUCUUUGGAAAGGUGAAGGGGCAGUAG |
| | | UAAUACAAGAUAAGAGUGACAUAAAAGUAGUGCCAAGAAGAAAAGCAAAG |
| | | AUUAUCAGGGAUUAUGGAAAACAGAUGGCAGGUGAUGAUUGUGUGGCAAG |
| | | UAGACAGGAUGAGGAUUAGAACAUGGAAAAGUUUAGUAAAACACCAUAUG |
| | | UAUGUUUCAAAGAAAGCUAAGGGAUGGUUUUAUAGACAUCACUAUGAAAG |
| | | CACUCAUCCAAGAAUAAGUUCAGAAGUACAUAUCCCACUAGGGGAUGCUA |
| | | GCUUGGUAGUAACAACAUAUUGGGGUCUACAUACAGGAGAAAGAGACUGG |
| | | CAUUUGGGUCAGGGAGUCUCCAUAGAAUGGAGGAAAAGGAGAUACAGCAC |
| | | ACAAGUAGACCCUGACCUAGCAGACCAACUAAUUCAUCUGUACUACUUUG |
| | | AUUGUUUUUCAGAAUCUGCUAUAAGAAAUGCCAUAUUAGGACAUAGAGUU |
| | | AGUCCUAGGUGUGAAUAUCAAGCAGGACAUAACAAGGUAGGAUCUCUACA |
| | | GUACUUGGCACUAGCAGCAUUAGUAACACCAAGAAAGAUAAAGCCACCUU |
| | | UGCCUAGUGUUGCGAAACUGACAGAGGACAGAUGGAACAAGUCCCACAAG |
| | | ACCAGGGGCCACAGAGGGAGCCAUACAAUGAAUGGACACUAAAGCUUUUA |
| | | GAGGAGCUUAAGAAUGAAGCUGUCAGACAUUUCCCUAGACCAUGGCUUCA |
| | | UGGCCUAGGGCAAUAUAUCUAUGAAACUUAUGAGGAUACUUGGGCAGGAG |
| | | UGGAAGCCAUAAUAAGAAUUCUGCAACAAUUGCUGCUUAUUCAUUUCAGA |
| | | AUUGGGUGUCAACAUAGCAGAAUAGGCAUUAUUCGACAGAGGAGAACAAG |
| | | AAAUGGAGCCAGUAGAUCCUAGACUAGAGCCCUGGAAGCAUCCAGGAAGU |
| | | CAGCCUAAGACUGCCUGUACCAAUUGCUAUUGCAAAAAGUGUUGCUUGCA |
| | | UUGCCAAGUUUGCUUCAUAACAAAAGGCUUAGGCAUCUCCUAUGGCAGGA |
| | | AGAAGCGGAAAAAGCGACGAAGAUCUCCUCAACACAGUCAGACUGAUCAA |
| | | GCUUCUCUAUCAAAGCAGUAAGUAGUACAUGUAAUGCAACCUUUAGUAAU |
| | | AUUAGCAAUAGUAGCAUUAGUAGUAGCACUAAUAAUAGUCAUAGUUGUAU |
| | | GGUCCAUUGUAUUAAUAGAAUAUAGAAAAAUAUUAAGACAAAAGAAAAUA |
| | | GACAGGUUAAUUGAUAGAAUAAGAGAAAAAGCAGAAGACAGUGGCAAUGA |
| | | GAGUGAUGGGGAUCAGGAAGAAUUAUCAGCACUUGUGGAAAGGGGGCACC |
| | | UUGCUCCUUGGGAUAUUGAUGAUCUGUAGUGCUGCAGAACAAUUGUGGGU |
| | | CACAGUCUAUUAUGGGGUACCUGUGUGGAAAGAAGCAAACACCACUCUAU |
| | | UUUGUGCAUCAGAUGCUAAGGCAUAUGAUACAGAGGUACAUAAUGUUUGG |
| | | GCCACACAUGCCUGUGUACCCACAGACCCCAACCCACAAGAAAUACUAUU |
| | | GGAAAAUGUGACAGAAGAUUUUAACAUGUGGAAAAAUAACAUGGUAGAAC |
| | | AGAUGCAUGAGGAUAUAAUCAGUUUAUGGGAUCAAAGUCUAAAGCCAUGU |
| | | GUAAAAUUAACCCCACUCUGUGUUACUUUACAUUGCACUGAUUUGAAGAA |
| | | UGGUACUAAUUUGAAGAAUGGUACUAAAAUCAUUGGGAAAUCAAUAAGAG |
| | | GAGAAAUAAAAAACUGCUCUUUCAAUGUCACCAAAAACAUAAUAGAUAAG |
| | | GUGAAAAAAGAAUAUGCGCUUUUCUAUAGACAUGAUGUAGUACCAAUAGA |
| | | UAGGAAUAUUACUAGCUAUAGGUUAAUAAGUUGUAACACCUCAACCCUUA |
| | | CACAGGCCUGUCCAAAGGUAUCCUUUGAGCCAAUUCCCAUACAUUAUUGU |
| | | GCCCCGGCUGGUUUUGCGAUUCUAAAAUGUAAAGAUAAGAAGUUCAAUGG |
| | | AACGGGACCAUGUACAAAUGUCAGUACAGUACAAUGUACACAUGGAAUUA |
| | | GGCCAGUAGUAUCAACUCAACUGCUGUUAAAUGGAAGUCUAGCAGAAGAA |
| | | GAGGUAGUAAUUAGAUCUAGCAAUUUCACGGACAAUGCUAAAAUCAUAAU |
| | | AGUACAGCUGAAUGAAACUGUAGAAAUUAAUUGUACAAGACCCAACAACA |
| | | AUACAAGAAAAGGGAUAACUCUAGGACCAGGGAGAGUAUUUUAUACAACA |
| | | GGAAAAAUAGUAGGAGAUAUAAGAAAAGCACAUUGUAACAUUAGUAAAGU |
| | | AAAAUGGCAUAACACUUUAAAAAGGGUAGUUAAAAAAUUAAGAGAAAAAU |
| | | UUGAAAAUAAAACAAUAAUCUUUAAUAAAUCCUCAGGGGGGGACCCAGAA |
| | | AUUGUAAUGCACAGCUUUAAUUGUGGAGGGGAAUUUUUCUACUGUAAUAC |
| | | AAAAAAACUGUUUAAUAGUACUUGGAAUGGUACUGAAGGGUCAUAUAACA |
| | | UUGAAGGAAAUGACACUAUCACACUCCCAUGCAGAAUAAAACAAAUUAUA |
| | | AACAUGUGGCAGGAAGUAGGAAAAGCAAUGUAUGCCCCUCCCAUCAGUGG |
| | | ACAAAUUUGGUGCUCAUCAAAUAUUACAGGGCUGCUACUAACAAGAGAUG |
| | | GUGGUAAGAACAGCAGCACCGAAAUCUUCAGACCUGGAGGAGGAGAUAUA |
| | | AGGGACAAUUGGAGAAGUGAAUUAUAUAAAUAUAAAGUAGUAAGAGUUGA |
| | | ACCAUUAGGAAUAGCACCCACCAAGGCAAAAAGAAGAGUGGUGCAGAGAG |
| | | AAAAAAGAGCAGUGGGAAUAGGAGCUGUGUUCCUUGGGUUCUUGGGAGCA |
| | | GCAGGAAGCACUAUGGGCGCAGCGUCAAUAACGCUGACGGUACAGGCCAG |
| | | ACAAUUAUUGUCUGGUAUAGUGCAACAGCAGAACAAUUUGCUGAGGGCUA |
| | | UUGAAGCGCAACAGCAUAUGUUGCAACUCACAGUCUGGGGCAUCAAGCAG |
| | | CUCCAGGCAAGAGUCCUGGCUGUGGAAAGAUACCUACAGGAUCAACAGCU |
| | | CCUGGGGAUUUGGGGUUGCUCUGGAAAACUCAUCUGCACCACUACUGUGC |
| | | CUUGGAAUACUAGUUGGAGUAAUAAAUCUCUGGAUACAAUUUGGGGUAAC |
| | | AUGACCUGGAUGCAGUGGGAAAAAGAAAUUAACAAUUACACAGGCUUAAU |
| | | AUACAACUUGAUUGAGGAAUCGCAGAACCAACAAGAAAAGAAUGAACAAG |
| | | AAUUAUUGGCAUUAGAUAAAUGGGCAAGUUUGUGGAAUUGGUUUAACAUA |
| | | UCAAACUGGCUGUGGUAUAUAAAAAUAUUCAUAAUGAUAGUAGGAGGCUU |
| | | GAUAGGUUUAAGAAUAGUUUUCAGUGUACUUUCUAUAGUGAAUAGAGUUA |
| | | GGCAGGGAUACUCACCAUUAUCGUUUCAGACCCGCUUCCCAGCCUCGAGG |
| | | GGACCCGACAGGCCCGAAGGAAUCGAAGAAGAAGGUGGAGACAGAGACAG |
| | | AGACAGAUCCAGUCCAUUAGUGGAUGGAUUCUUAGCAAUCAUCUGGGUCG |
| | | ACCUGCGGAGCCUGUUCCUCUUCAGCUACCACCGCUUGAGAGACUUACUC |
| | | UUGAUUGUAACGAGGAUUGUGGAACUUCUGGGACGCAGGGGGUGGGAACU |
| | | CCUCAAAUACUUGUGGAAUCUCCUGCAGUAUUGGAGUCAGGAACUAAAGA |
| | | AUAGUGCUGUUAGCUUGCUUAACGCCACAGCCAUAGCAGUAGGUGAGGGA |
| | | ACAGAUAGAAUUAUAGAAAUAUUACAAAGAGCUGGUAGAGCUAUUCUCAA |
| | | CAUACCUACGAGAAUAAGACAGGGCUUAGAAAGGGCUUUGCUAUAAGCUU |
| | | AUGGGUGGAGCUAUUUCCAUGAGGCGGUCCAGGCCGUCUGGAAAUCUGUA |
| | | CGAGAGACUCUUGCGGGCGCGUGGGGAGACUUAUGGAAAACUCUUAGGAG |
| | | AGGUAAAAGAUGGAUACUCGCAAUCCCCAGGAGGAUUAGACAAGGGCUUG |
| | | AGCUCACUCUCUUGUGAGGGACAAAAAUACAAUCAGGGACAGUAUAUGAA |
| | | UACUCCAUGGAGAAACCCAGCUAAAGAGAGAGAAAAAUUAGCAUACAGAA |
| | | AACAAAAUAUGGAUGAUAUAGAUAAGGAAGAUGAUGACUUGGUAGGGGUA |
| | | UCAGUGAGGCCAAAAGUUCCCCUAAGAACAAUGAGUUACAAAUUGGCAAU |
| | | AGACAUGUCUCAUUUUAUAAAGGAAAAGGGGGGACUGGAAGGGAUUUAUU |
| | | ACAGUGCAAGAAGACAUAGAAUCUUAGACAUAUACUUAGAAAAGGAAGAA |
| | | GGCAUCAUACCAGAUUGGCAGGAUUACACCUCAGGACCAGGAAUUAGAUA |
| | | CCCAAAGACAUUUGGCUGGCUAUGGAAAUUAGUCCCUGUAAAUGUAUCAG |
| | | AUGAGGCACAGGAGGAUGAGGAGCAUUAUUUAAUGCAUCCAGCUCAAACU |
| | | UCCCAGUGGGAUGACCCUUGGGGAGAGGUUCUAGCAUGGAAGUUUGAUCC |
| | | AACUCUGGCCUACACUUAUGAGGCAUAUGUUAGAUACCCAGAAGAGUUUG |
| | | GAAGCAAGUCAGGCCUGUCAGAGGAAGAGGUUAAAAGAAGGCUAACCGCA |
| | | AGAGGCCUUCUUAACAUGGCUGACAAGAAGGAAACUCGCUGAAUUCGAGC |
| | | UAUCUACAGGGGACUUUCCGCUGGGGACUUUCCAGGGAGGCGUGGCCUGG |
| | | GCGGGACCGGGGAGUGGCGAGCCCUCAGAUGCUGCAUAUAAGCAGCCGCU |
| | | UUUGCCUGUACUGGGUCUCUCUAGUUAGACCAGAUCUGAGCCUGGGAGCU |
| | | CUCUGGCUAGCUGAGAACCCACUGCUUAAGCCUCAAUAAAGCUUGCCUUG |
| | | AGUGCUUUAAGUAGUGUGUGCCCGUCUGUUGUGUGACUCUGGUAACUAGA |
| | | GAUCCCUCAGACCAUUUUAGUCAGUGUGGAAAAUCUCUAGCA |
| SEQ ID | clone 1.27 | UGGAAGGGAUUUAUUACAGUGCAAGAAGACAUAGAAUCUUAGACAUAUAC |
| NO: 52 | RNA | UUAGAAAAGGAAGAAGGCAUCAUACCAGAUUGGCAGGAUUACACCUCAGG |
| | | ACCAGGAAUUAGAUACCCAAAGACAUUUGGCUGGCUAUGGAAAUUAGUCC |
| | | CUGUAAAUGUAUCAGAUGAGGCACAGGAGGAUGAGGAGCAUUAUUUAAUG |
| | | CAUCCAGCUCAAACUUCCCAGUGGGAUGACCCUUGGGGAGAGGUUCUAGC |
| | | AUGGAAGUUUGAUCCAACUCUGGCCUACACUUAUGAGGCAUAUGUUAGAU |
| | | ACCCAGAAGAGUUUGGAAGCAAGUCAGGCCUGUCAGAGGAAGAGGUUAAA |
| | | AGAAGGCUAACCGCAAGAGGCCUUCUUAACAUGGCUGACAAGAAGGAAAC |
| | | UCGCUGAAUUCGAGCUAUCUACAGGGGACUUUCCGCUGGGGACUUUCCAG |
| | | GGAGGCGUGGCCUGGGCGGGACCGGGGAGUGGCGAGCCCUCAGAUGCUGC |
| | | AUAUAAGCAGCCGCUUUUGCCUGUACUGGGUCUCUCUAGUUAGACCAGAU |
| | | CUGAGCCUGGGAGCUCUCUGGCUAGCUGAGAACCCACUGCUUAAGCCUCA |
| | | AUAAAGCUUGCCUUGAGUGCUUUAAGUAGUGUGUGCCCGUCUGUUGUGUG |
| | | ACUCUGGUAACUAGAGAUCCCUCAGACCAUUUUAGUCAGUGUGGAAAAUC |
| | | UCUAGCAGUGGCGCCCGAACAGGGACCGGAAAGCGAAAGAGAAACCAGAG |
| | | AAGCUCUCUCGACGCAGGACUCGGCUUGCUGAAGCGCGCACGGCAAGCGG |
| | | CGAGGGGCAGCGACCGGUGAGUACGCUAAAAAUUUUGACUAGCGGAGGCU |
| | | AGAAGGAGAGAGAUGGGUGCGAGAGCGUCAGUAUUAAGCGGGGGAAAAUU |
| | | GGAUGCAUGGGAAAAAAUUCGGUUACGGCCAGGAGGAAAGAAAAAAUAUA |
| | | GACUAAAACAUCUAGUAUGGGCAAGCAGGGAGCUAGAACGAUUUGCACUU |
| | | AAUCCUGGCCUUUUAGAGACAUCAGAUGGCUGUAAACAAAUAAUAGGACA |
| | | GCUACAACCAGCUAUCCGGACAGGAUCAGAAGAACUUAGAUCAUUAUUUA |
| | | AUACAGUAGCAACCCUCUAUUGUGUACAUGAAAGGAUAGAGGUAAAAGAC |
| | | ACCAAGGAAGCUUUAGAGAAGAUAGAGGAAGAGCAAAACAAAAGUAAGAA |
| | | AAAAGCACAGCAAGCAGCAGCUGACACAGGACACAGCAAUCAGGUCAGCC |
| | | AAAAUUACCCUAUAGUGCAGAACAUCCAGGGGCAAAUGGUACAUCAGGCC |
| | | CUAUCACCUAGAACUUUAAAUGCGUGGGUAAAAGUAGUAGAAGAGAAGGC |
| | | UUUUAGCCCAGAAGUAAUACCCAUGUUUUCAGCAUUAUCAGAAGGAGCCA |
| | | CCCCACAAGAUUUAAACACCAUGCUAAACACAGUGGGGGGACAUCAAGCA |
| | | GCCAUGCAAAUGUUAAAAGAGACCAUCAAUGAGGAAGCUGCAGAAUGGGA |
| | | UAGAGUGCAUCCAGUGCAUGCAGGGCCUAUUGCACCAGGCCAGAUGAGAG |
| | | AGCCAAGGGGAAGUGACAUAGCAGGAACUACUAGUACCCUUCAGGAACAA |
| | | AUAGGAUGGAUGACACAUAAUCCACCUAUCCCAGUAGGAGAAAUCUAUAA |
| | | AAGAUGGAUAAUCCUGGGAUUAAAUAAAAUAGUAAGAAUGUAUAGCCCUA |
| | | CCAGCAUUCUGGACAUAAGACAAGGACCAAAGGAACCCUUUAGAGACUAU |
| | | GUAGACCGGUUCUAUAAAACCCUAAGAGCCGAGCAAGCUACACAGGAGGU |
| | | AAAAAAUUGGAUGACAGAAACCUUGUUGGUCCAAAAUGCGAACCCAGAUU |
| | | GUAAAACUAUUUUAAAAGCAUUGGGACCAGCAGCCACACUAGAAGAAAUG |
| | | AUGACAGCAUGUCAGGGAGUGGGAGGACCCGGCCAUAAAGCAAGAGUUUU |
| | | GGCUGAAGCAAUGAGCCAAGUAACAAACUCAGCUACCAUAAUGAUGCAGA |
| | | GAGGCAAUUUUAGGAACCAAAGAAAAACUGUUAAGUGUUUCAAUUGUGGC |
| | | AAAGAAGGGCACAUAGCCAAAAAUUGCAGGGCUCCUAGGAAAAAGGGCUG |
| | | UUGGAAAUGUGGAAAGGAAGGACACCAAAUGAAAGAUUGUACUGAGAGAC |
| | | AGGCUAAUUUUUUAGGGAAGAUCUGGCCUUCCCACAAGGGAAGGCCAGGA |
| | | AAUUUUCUUCAGAGCAGACCAGAGCCAACAGCCCCAUCAGAAGAGAGCGU |
| | | CAAGUUUGGAGAAGAGACAACAACUCCCUCUCAGAAGCAGGAGCCGAUAG |
| | | ACAAGGAACUGUAUCCUUUAACUUCCCUCAGAUCACUCUUUGGCAACGAC |
| | | CCCUCGUCACAAUAAAGAUAGGGGGGCAACUAAAGGAAGCUCUAUUAGAU |
| | | ACAGGAGCAGAUGAUACAGUAUUAGAAGACAUGGAUUUGCCAGGAAGAUG |
| | | GAAACCAAAAAUGAUAGGGGGAAUUGGAGGUUUUAUCAAAGUAAGACAGU |
| | | AUGAUCAGAUACCCAUAGAUAUCUGUGGACAUAAAGCUGUAGGUACAGUA |
| | | UUAGUAGGACCUACACCUGUCAACAUAAUUGGAAGAAAUCUGUUGACUCA |
| | | GAUUGGUUGCACUUUAAAUUUUCCCAUUAGUCCUAUUGAAACUGUACCAG |
| | | UAAAAUUAAAGCCAGGAAUGGAUGGCCCAAAAGUCAAACAAUGGCCAUUG |
| | | ACAGAAGAAAAAAUAAAAGCAUUAGUAGAAAUUUGUACAGAAAUGGAAAA |
| | | GGAAGGAAAGAUUUCAAAAAUUGGGCCUGAAAAUCCAUACAAUACUCCAG |
| | | UAUUUGCCAUAAAGAAAAAAGACAGUACUAAAUGGAGAAAAUUAGUAGAU |
| | | UUCAGAGAACUUAAUAGGAAAACUCAAGACUUCUGGGAAGUUCAAUUAGG |
| | | AAUACCACAUCCCGCAGGGUUAAAAAAGAAAAAAUCAGUAACAGUACUGG |
| | | AUGUGGGUGAUGCAUAUUUUUCAGUUCCCUUAGAUAAAGACUUCAGGAAG |
| | | UAUACUGCAUUUACCAUACCUAGUAUAAACAAUGAGACACCAGGGAUUAG |
| | | AUAUCAGUACAAUGUGCUUCCACAGGGAUGGAAAGGAUCACCAGCAAUAU |
| | | UCCAAAGUAGCAUGACAAAAACCUUAGAGCCUUUUAGAAAACAAAAUCCA |
| | | GACAUAAUUAUCUAUCAAUACAUGGAUGAUUUGUAUGUAGGAUCUGACUU |
| | | AGAAAUAGGGCAGCAUAGAACAAAAAUAGAGGAACUGAGACAACAUCUGU |
| | | UAAAGUGGGGAUUUACCACACCAGACAAAAAACAUCAGAAAGAACCUCCA |
| | | UUCCUUUGGAUGGGUUAUGAACUCCAUCCUGAUAAAUGGACAGUACAGCC |
| | | UAUAGUGCUGCCAGAAAAAGACAGCUGGACUGUCAAUGACAUACAGAAGU |
| | | UAGUGGGAAAAUUAAAUUGGGCAAGUCAAAUUUAUGCAGGGAUUAAAGUA |
| | | AAGCAAUUAUGUAAACUCCUUAGGGGAACCAAAGCACUUACAGAAGUAAU |
| | | ACCACUAACAAAAGAAGCAGAGCUAGAACUGGCAGAAAACAGGGAGAUUU |
| | | UAAAGGAACCAGUACAUGGAGUGUAUUAUGACCCAUCAAAAGACUUAAUA |
| | | GUAGAAAUACAGAAGCAGGGGCAAGGCCAAUGGACAUAUCAAAUUUUUCA |
| | | AGAGCCAUUUAAAAAUCUGAAAACAGGAAAAUAUGCAAAAACGAGGGGUG |
| | | CCCACACUAAUGAUGUAAAACAAUUAACAGAGGCAGUGCAAAAAAUAGCC |
| | | AAUGAAAGCAUAGUAAUAUGGGGAAAGAUUCCUAAAUUUAAAUUACCCAU |
| | | ACAAAAAGAAACAUGGGAAACAUGGUGGACAGAGUAUUGGCAAGCCACCU |
| | | GGAUUCCUGAGUGGGAGUUUGUCAAUACCCCUCCCUUAGUGAAAUUAUGG |
| | | UACCAGUUAGAAAAAGAACCCAUAGUAGGAGCAGAAACUUUCUAUGUAGA |
| | | UGGGGCAGCUAACAGGGAGACUAAAUUAGGAAAAGCAGGAUAUGUUACUA |
| | | GCAGAGGAAGGCAAAAAGUUGUCUCCCUAACAGACACAACAAAUCAGAAA |
| | | ACUGAGUUACAAGCAAUUCACCUAGCUUUGCAGGAUUCAGGAUUAGAAGU |
| | | AAACAUAGUAACAGACUCACAAUAUGCAUUAGGAAUCAUUCAAGCACAAC |
| | | CAGAUAAAAGUGAAUCAGAGUUAGUCAGUCAAAUAAUAGAACAGCUAAUA |
| | | AAAAAGGAAAAAGUCUACCUGGCAUGGGUACCAGCACACAAAGGAAUUGG |
| | | AGGAAAUGAACAGGUAGAUAAAUUAGUCAGUGCUGGAAUCAGGAGAGUAC |
| | | UAUUUCUAGAUGGAAUAGAGAAGGCCCAAGAAGAACAUGAGAAAUAUCAU |
| | | AAUAAUUGGAGAGCAAUGGCUAGUGAAUUUAACCUGCCAGCUGUAGUAGC |
| | | AAAAGAGAUAGUAGCCUGCUGUGAUAAGUGCCAGGUAAAAGGAGAAGCCA |
| | | UGCAUGGACAAGUAGACUGCAGUCCAGGAAUAUGGCAACUAGAUUGUACA |
| | | CAUUUAGAAGGAAAAGUUAUCCUGGUAGCAGUUCAUGUAGCCAGUGGAUA |
| | | UAUAGAAGCAGAGGUUAUUCCAGCAGAGACAGGACAGGAAACAGCAUACU |
| | | UUAUUUUAAAAUUAGCAGGAAGAUGGCCAGUAAAAACAAUACAUACAGAC |
| | | AAUGGCAGUAAUUUCACCAGUACUACGGUUAAGGCCGCCUGUUGGUGGGC |
| | | AGGGAUCAAGCAGGAAUCUGGCAUUCCCUACAAUCCCCAAAGUCAAGGAG |
| | | UAGUAGAAUCUAUGAAUAAAGAAUUAAAGAAAAUUAUAGAACAAGUAAGA |
| | | GAUCAGGCUGAACAUCUUAAGACAGCAGUACAAAUGGCAGUAUUCAUUCA |
| | | CAAUUUUAAAAGAAAAGGGGGGAUUGGGGGGUACAGUGCAGGGGAAAGAA |
| | | UAGUAGACAUAAUAGCAUCAGACAUACAAACUAAAGAACUACAAAAACAA |
| | | AUUACAAAAAUUCAAAAUUUUCGGGUUUAUUACAGGGACAGCAGAGAUCC |
| | | ACUUUGGAAAGGACCAGCAAAGCUUCUUUGGAAAGGUGAAGGGGCAGUAG |
| | | UAAUACAAGAUAAGAGUGACAUAAAAGUAGUGCCAAGAAGAAAAGCAAAG |
| | | AUUAUCAGGGAUUAUGGAAAACAGAUGGCAGGUGAUGAUUGUGUGGCAAG |
| | | UAGACAGGAUGAGGAUUAGAACAUGGAAAAGUUUAGUAAAACACCAUAUG |
| | | UAUGUUUCAAAGAAAGCUAAGGGAUGGUUUUAUAGACAUCACUAUGAAAG |
| | | CACUCAUCCAAGAAUAAGUUCAGAAGUACAUAUCCCACUAGGGGAUGCUA |
| | | GCUUGGUAGUAACAACAUAUUGGGGUCUACAUACAGGAGAAAGAGACUGG |
| | | CAUUUGGGUCAGGGAGUCUCCAUAGAAUGGAGGAAAAGGAGAUACAGCAC |
| | | ACAAGUAGACCCUGACCUAGCAGACCAACUAAUUCAUCUGUACUACUUUG |
| | | AUUGUUUUUCAGAAUCUGCUAUAAGAAAUGCCAUAUUAGGACAUAGAGUU |
| | | AGUCCUAGGUGUGAAUAUCAAGCAGGACAUAACAAGGUAGGAUCUCUACA |
| | | GUACUUGGCACUAGCAGCAUUAGUAACACCAAGAAAGAUAAAGCCACCUU |
| | | UGCCUAGUGUUGCGAAACUGACAGAGGACAGAUGGAACAAGUCCCACAAG |
| | | ACCAAGGGCCACAGAGGGAGCCAUACAAUGAAUGGACACUAAAGCUUUUA |
| | | GAGGAGCUUAAGAAUGAAGCUGUCAGACAUUUCCCUAGACCAUGGCUUCA |
| | | UGGCCUAGGGCAAUAUAUCUAUGAAACUUAUGAGGAUACUUGGGCAGGAG |
| | | UGGAAGCCAUAAUAAGAAUUCUGCAACAAUUGCUGCUUAUUCAUUUCAGA |
| | | AUUGGGUGUCAACAUAGCAGAAUAGGCAUUAUUCGACAGAGGAGAACAAG |
| | | AAAUGGAGCCAGUAGAUCCUAGACUAGAGCCCUGGAAGCAUCCAGGAAGU |
| | | CAGCCUAAGACUGCCUGUACCAAUUGCUAUUGCAAAAAGUGUUGCUUGCA |
| | | UUGCCAAGUUUGCUUCAUAACAAAAGGCUUAGGCAUCUCCUAUGGCAGGA |
| | | AGAAGCGGAAAAAGCGACGAAGAUCUCCUCAACACAGUCAGACUGAUCAA |
| | | GCUUCUCUAUCAAAGCAGUAAGUAGUACAUGUAAUGCAACCUUUAGUAAU |
| | | AUUAGCAAUAGUAGCAUUAGUAGUAGCACUAAUAAUAGUCAUAGUUGUAU |
| | | GGUCCAUUGUAUUAAUAGAAUAUAGAAAAAUAUUAAGACAAAAGAAAAUA |
| | | GACAGGUUAAUUGAUAGAAUAAGAGAAAAAGCAGAAGACAGUGGCAAUGA |
| | | GAGUGAUGGGGAUCAGGAAGAAUUAUCAGCACUUGUGGAAAGGGGGCACC |
| | | UUGCUCCUUGGGAUAUUGAUGAUCUGUAGUGCUGCAGAACAAUUGUGGGU |
| | | CACAGUCUAUUAUGGGGUACCUGUGUGGAAAGAAGCAAACACCACUCUAU |
| | | UUUGUGCAUCAGAUGCUAAGGCAUAUGAUACAGAGGUACAUAAUGUUUGG |
| | | GCCACACAUGCCUGUGUACCCACAGACCCCAACCCACAAGAAAUACUAUU |
| | | GGAAAAUGUGACAGAAGAUUUUAACAUGUGGAAAAAUAACAUGGUAGAAC |
| | | AGAUGCAUGAGGAUAUAAUCAGUUUAUGGGAUCAAAGUCUAAAGCCAUGU |
| | | GUAAAAUUAACCCCACUCUGUGUUACUUUACAUUGCACUGAUUUGAAGAA |
| | | UGGUACUAAUUUGAAGAAUGGUACUAAAAUCAUUGGGAAAUCAAUAAGAG |
| | | GAGAAAUAAAAAACUGCUCUUUCAAUGUCACCAAAAACAUAAUAGAUAAG |
| | | GUGAAAAAAGAAUAUGCGCUUUUCUAUAGACAUGAUGUAGUACCAAUAGA |
| | | UAGGAAUAUUACUAGCUAUAGGUUAAUAAGUUGUAACACCUCAACCCUUA |
| | | CACAGGCCUGUCCAAAGGUAUCCUUUGAGCCAAUUCCCAUACAUUAUUGU |
| | | GCCCCGGCUGGUUUUGCGAUUCUAAAAUGUAAAGAUAAGAAGUUCAAUGG |
| | | AACGGGACCAUGUACAAAUGUCAGUACAGUACAAUGUACACACGGAAUUA |
| | | GGCCAGUAGUAUCAACUCAACUGCUGUUAAAUGGAAGUCUAGCAGAAGAA |
| | | GAGGUAGUAAUUAGAUCUAGCAAUUUCACGGACAAUGCUAAAAUCAUAAU |
| | | AGUACAGCUGAAUGAAACUGUAGAAAUUAAUUGUACAAGACCCAACAACA |
| | | AUACAAGAAAAGGGAUAACUCUAGGACCAGGGAGAGUAUUUUAUACAACA |
| | | GGAAAAAUAGUAGGAGAUAUAAGAAAAGCACAUUGUAACAUUAGUAAAGU |
| | | AAAAUGGCAUAACACUUUAAAAAGGGUAGUUAAAAAAUUAAGAGAAAAAU |
| | | UUGAAAAUAAAACAAUAAUCUUUAAUAAAUCCUCAGGGGGGGACCCAGAA |
| | | AUUGUAAUGCACAGCUUUAAUUGUGGAGGGGAAUUUUUCUACUGUAAUAC |
| | | AAAAAAACUGUUUAAUAGUACUUGGAAUGGUACUGAAGGGUCAUAUAACA |
| | | UUGAAGGAAAUGACACUAUCACACUCCCAUGCAGAAUAAAACAAAUUAUA |
| | | AACAUGUGGCAGGAAGUAGGAAAAGCAAUGUAUGCCCCUCCCAUCAGUGG |
| | | ACAAAUUUGGUGCUCAUCAAAUAUUACAGGGCUGCUACUAACAAGAGAUG |
| | | GUGGUAAGAACAGCAGCACCGAAAUCUUCAGACCUGGAGGAGGAGAUAUA |
| | | AGGGACAAUUGGAGAAGUGAAUUAUAUAAAUAUAAAGUAGUAAGAGUUGA |
| | | ACCAUUAGGAAUAGCACCCACCAAGGCAAAAAGAAGAGUGGUGCAGAGAG |
| | | AAAAAAGAGCAGUGGGAAUAGGAGCUGUGUUCCUUGGGUUCUUGGGAGCA |
| | | GCAGGAAGCACUAUGGGCGCAGCGUCAAUAACGCUGACGGUACAGGCCAG |
| | | ACAAUUAUUGUCUGGUAUAGUGCAACAGCAGAACAAUUUGCUGAGGGCUA |
| | | UUGAAGCGCAACAGCAUAUGUUGCAACUCACAGUCUGGGGCAUCAAGCAG |
| | | CUCCAGGCAAGAGUCCUGGCUGUGGAAAGAUACCUACAGGAUCAACAGCU |
| | | CCUGGGGAUUUGGGGUUGCUCUGGAAAACUCAUCUGCACCACUACUGUGC |
| | | CUUGGAAUACUAGUUGGAGUAAUAAAUCUCUGGAUACAAUUUGGGGUAAC |
| | | AUGACCUGGAUGCAGUGGGAAAAAGAAAUUAACAAUUACACAGGCUUAAU |
| | | AUACAACUUGAUUGAAGAAUCGCAGAACCAACAAGAAAAGAAUGAACAAG |
| | | AAUUAUUGGCAUUAGAUAAAUGGGCAAGUUUGUGGAAUUGGUUUAACAUA |
| | | UCAAACUGGCUGUGGUAUAUAAAAAUAUUCAUAAUGAUAGUAGGAGGCUU |
| | | GAUAGGUUUAAGAAUAGUUUUCAGUGUACUUUCUAUAGUGAAUAGAGUUA |
| | | GGCAGGGAUACUCACCAUUAUCGUUUCAGACCCGCUUCCCAGCCUCGAGG |
| | | GGACCCGACAGGCCCGAAGGAAUCGAAGAAGAAGGUGGAGACAGAGACAG |
| | | AGACAGAUCCAGUCCAUUAGUGGAUGGAUUCUUAGCAAUCAUCUGGGUCG |
| | | ACCUGCGGAGCCUGUUCCUCUUCAGCUACCACCGCUUGAGAGACUUACUC |
| | | UUGAUUGUAACGAGGAUUGUGGAACUUCUGGGACGCAGGGGGUGGGAACU |
| | | CCUCAAAUACUUGUGGAAUCUCCUGCAGUAUUGGAGUCAGGAACUAAAGA |
| | | AUAGUGCUGUUAGCUUGCUUAACGCCACAGCCAUAGCAGUAGGUGAGGGA |
| | | ACAGAUAGAAUUAUAGAAAUAUUACAAAGAGCUGGUAGAGCUAUUCUCAA |
| | | CAUACCUACGAGAAUAAGACAGGGCUUAGAAAGGGCUUUGCUAUAAGCUU |
| | | AUGGGUGGAGCUAUUUCCAUGAGGCGGUCCAGGCCGUCUGGAAAUCUGUA |
| | | CGAGAGACUCUUGCGGGCGCGUGGGGAGACUUAUGGAAAACUCUUAGGAG |
| | | AGGUAAAAGAUGGAUACUCGCAAUCCCCAGGAGGAUUAGACAAGGGCUUG |
| | | AGCUCACUCUCUUGUGGGGGACAAAAAUACAAUCAGGGACAGUAUAUGAA |
| | | UACUCCAUGGAGAAACCCAGCUAAAGAGAGAGAAAAAUUAGCAUACAGAA |
| | | AACAAAAUAUGGAUGAUAUAGAUAAGGAAGAUGAUGACUUGGUAGGGGUA |
| | | UCAGUGAGGCCAAAAGUUCCCCUAAGAACAAUGAGUUACAAAUUGGCAAU |
| | | AGACAUGUCUCAUUUUAUAAAAGAAAAGGGGGGACUGGAAGGGAUUUAUU |
| | | ACAGUGCAAGAAGACAUAGAAUCUUAGACAUAUACUUAAAAAAGGAAGAA |
| | | GGCAUCAUACCAGAUUGGCAGGAUUACACCUCAGGACCAGGAAUUAGAUA |
| | | CCCAAAGACAUUUGGCUGGCUAUGGAAAUUAGUCCCUGUAAAUGUAUCAG |
| | | AUGAGGCACAGGAGGAUGAGGAGCAUUAUUUAAUGCAUCCAGCUCAAACU |
| | | UCCCAGUGGGAUGACCCUUGGGGAGAGGUUCUAGCAUGGAAGUUUGAUCC |
| | | AACUCUGGCCUACACUUAUGAGGCAUAUGUUAGAUACCCAGAAGAGUUUG |
| | | GAAGCAAGUCAGGCCUGUCAGAGGAAGAGGUUAAAAGAAGGCUAACCGCA |
| | | AGAGGCCUUCUUAACAUGGCUGACAAGAAGGAAACUCGCUGAAUUCGAGC |
| | | UAUCUACAGGGGACUUUCCGCUGGGGACUUUCCAGGGAGGCGUGGCCUGG |
| | | GCGGGACCGGGGAGUGGCGAGCCCUCAGAUGCUGCAUAUAAGCAGCCGCU |
| | | UUUGCCUGUACUGGGUCUCUCUAGUUAGACCAGAUCUGAGCCUGGGAGCU |
| | | CUCUGGCUAGCUGAGAACCCACUGCUUAAGCCUCAAUAAAGCUUGCCUUG |
| | | AGUGCUUUAAGUAGUGUGUGCCCGUCUGUUGUGUGACUCUGGUAACUAGA |
| | | GAUCCCUCAGACCAUUUUAGUCAGUGUGGAAAAUCUCUAGCA |
| SEQ ID | clone 1.10 | UGGAAGGGAUUUAUUACAGUGCAAGAAGACAUAGAAUCUUAGACAUAUAC |
| NO: 53 | RNA | UUAGAAAAGGAAGAAGGCAUCAUACCAGAUUGGCAGGAUUACACCUCAGG |
| | | ACCAGGAAUUAGAUACCCAAAGACAUUUGGCUGGCUAUGGAAAUUAGUCC |
| | | CUGUAAAUGUAUCAGAUGAGGCACAGGAGGAUGAGGAGCAUUAUUUAAUG |
| | | CAUCCAGCUCAAACUUCCCAGUGGGAUGACCCUUGGGGAGAGGUUCUAGC |
| | | AUGGAAGUUUGAUCCAACUCUGGCCUACACUUAUGAGGCAUAUGUUAGAU |
| | | ACCCAGAAGAGUUUGGAAACAAGUCAGGCCUGUCAGAGGAAGAGGUUAAA |
| | | AGAAGGCUAACCGCAAGAGGCCUUCUUAACAUGGCUGACAAGAAGGAAAC |
| | | UCGCUGAAUUCGAGCUAUCUACAGGGGACUUUCCGCUGGGGACUUUCCAG |
| | | GGAGGCGUGGCCUGGGCGGGACCGGGGAGUGGCGAGCCCUCAGAUGCUGC |
| | | AUAUAAGCAGCCGCUUUUGCCUGUACUGGGUCUCUCUAGUUAGACCAGAU |
| | | CUGAGCCUGGGAGCUCUCUGGCUAGCUGAGAACCCACUGCUUAAGCCUCA |
| | | AUAAAGCUUGCCUUGAGUGCUUUAAGUAGUGUGUGCCCGUCUGUUGUGUG |
| | | ACUCUGGUAACUAGAGAUCCCUCAGACCAUUUUAGUCAGUGUGGAAAAUC |
| | | UCUAGCAGUGGCGCCCGAACAGGGACUUGAAAGCGAAAGAGAAACCAGAG |
| | | AAGCUCUCUCGACGCAGGACUCGGCUUGCUGAAGCGCGCACGGCAAGCGG |
| | | CGAGGGGCAGCGACCGGUGAGUACGCUAAAAAUUUUGACUAGCGGAGGCU |
| | | AGAAGGAGAGAGAUGGGUGCGAGAGCGUCAGUAUUAAGCGGGGGAAAAUU |
| | | GGAUGCAUGGGAAAAAAUUCGGUUACGGCCAGGAGGAAAGAAAAAAUAUA |
| | | GACUAAAACAUCUAGUAUGGGCAAGCAGGGAGCUAGAACGAUUUGCACUU |
| | | AAUCCUGGCCUUUUAGAGACAUCAGAUGGCUGUAAACAAAUAAUAGGACA |
| | | GCUACAACCAGCUAUCCGGACAGGAUCAGAAGAACUUAGAUCAUUAUUUA |
| | | AUACAGUAGCAACCCUCUAUUGUGUACAUGAAAGGAUAGAGGUAAAAGAC |
| | | ACCAAGGAAGCUUUAGAGAAGAUAGAGGAAGAGCAAAACAAAAGUAAGAA |
| | | AAAAGCACAGCAAGCAGCAGCUGACACAGGACACAGCAAUCAGGUCAGCC |
| | | AAAAUUACCCUAUAGUGCAGAACAUCCAGGGGCAAAUGGUACAUCAGGCC |
| | | CUAUCACCUAGAACUUUAAAUGCGUGGGUAAAAGUAGUAGAAGAGAAGGC |
| | | UUUUAGCCCAGAAGUAAUACCCAUGUUUUCAGCAUUAUCAGAAGGAGCCA |
| | | CCCCACAAGAUUUAAACACCAUGCUAAACACAGUGGGGGGACAUCAAGCA |
| | | GCCAUGCAAAUGUUAAAAGAGACCAUCAAUGAGGAAGCUGCAGAAUGGGA |
| | | UAGAGUGCAUCCAGUGCAUGCAGGGCCUAUUGCACCAGGCCAGAUGAGAG |
| | | AACCAAGGGGAAGUGACAUAGCAGGAACUACUAGUACCCUUCAGGAACAA |
| | | AUAGGAUGGAUGACACAUAAUCCACCUAUCCCAGUAGGAGAAAUCUAUAA |
| | | AAGAUGGAUAAUCCUGGGAUUAAAUAAAAUAGUAAGAAUGUAUAGCCCUA |
| | | CCAGCAUUCUGGACAUAAGACAAGGACCAAAGGAACCCUUUAGAGACUAU |
| | | GUAGACCGGUUCUAUAAAACCCUAAGAGCCGAGCAAGCUACACAGGAGGU |
| | | AAAAAAUUGGAUGACAGAAACCUUGUUGGUCCAAAAUGCGAACCCAGAUU |
| | | GUAAAACUAUUUUAAAAGCAUUGGGACCAGCAGCCACACUAGAAGAAAUG |
| | | AUGACAGCAUGUCAGGGAGUGGGAGGACCCGGCCAUAAAGCAAGAGUUUU |
| | | GGCUGAAGCAAUGAGCCAAGUAACAAAUUCAGCUACCAUAAUGAUGCAGA |
| | | GAGGCAAUUUUAGGAACCAAAGAAAAACUGUUAAGUGUUUCAAUUGUGGC |
| | | AAAGAAGGGCACAUAGCCAAAAAUUGCAGGGCUCCUAGGAAAAAGGGCUG |
| | | UUGGAAAUGUGGAAAGGAAGGACACCAAAUGAAAGAUUGUACUGAGAGAC |
| | | AGGCUAAUUUUUUAGGGAAGAUCUGGCCUUCCCACAAGGGAAGGCCAGGA |
| | | AAUUUUCUUCAGAGCAGACCAGAGCCAACAGCCCCAUCAGAAGAGAGCGU |
| | | CAGGUUUGGAGAAGAGACAACAACUCCCUCUCAGAAGCAGGAGCCGAUAG |
| | | ACAAGGAACUGUAUCCUUUAACUUCCCUCAGAUCACUCUUUGGCAACGAC |
| | | CCCUCGUCACAAUAAAGAUAGGGGGGCAACUAAAGGAAGCUCUAUUAGAU |
| | | ACAGGAGCAGAUGAUACAGUAUUAGAAGACAUGGAUUUGCCAGGAAGAUG |
| | | GAAACCAAAAAUGAUAGGGGGAAUUGGAGGUUUUAUCAAAGUAAGACAGU |
| | | AUGAUCAGAUACCCAUAGAUAUCUGUGGACAUAAAGCUGUAGGUACAGUA |
| | | UUAGUAGGACCUACACCUGUCAACAUAAUUGGAAGAAAUCUGUUGACUCA |
| | | GAUUGGUUGCACUUUAAAUUUUCCCAUUAGUCCUAUUGAAACUGUACCAG |
| | | UAAAAUUAAAGCCAGGAAUGGAUGGCCCAAAAGUCAAACAAUGGCCAUUG |
| | | ACAGAAGAAAAAAUAAAAGCAUUAGUAGAAAUUUGUACAGAAAUGGAAAA |
| | | GGAAGGAAAGAUUUCAAAAAUUGGGCCUGAAAAUCCAUACAAUACUCCAG |
| | | UAUUUGCCAUAAAGAAAAAAGACAGUACUAAAUGGAGAAAAUUAGUAGAU |
| | | UUCAGAGAACUUAAUAGGAAAACUCAAGACUUCUGGGAAGUUCAAUUAGG |
| | | AAUACCACAUCCCGCAGGGUUAAAAAAGAAAAAAUCAGUAACAGUACUGG |
| | | AUGUGGGUGAUGCAUAUUUUUCAGUUCCCUUAGAUAAAGACUUCAGGAAG |
| | | UAUACUGCAUUUACCAUACCUAGUAUAAACAAUGAGACACCAGGGAUUAG |
| | | AUAUCAGUACAAUGUGCUUCCACAGGGAUGGAAAGGAUCACCAGCAAUAU |
| | | UCCAAAGUAGCAUGACAAAAACCUUAGAGCCUUUUAGAAAACAAAAUCCA |
| | | GACAUAAUUAUCUAUCAAUACAUGGAUGAUUUGUAUGUAGGAUCUGACUU |
| | | AGAAAUAGGGCAGCAUAGAACAAAAAUAGAGGAACUGAGACAACAUCUGU |
| | | UAAAGUGGGGAUUUACCACACCAGACAAAAAACAUCAGAAAGAACCUCCA |
| | | UUCCUUUGGAUGGGUUAUGAACUCCAUCCUGAUAAAUGGACAGUACAGCC |
| | | UAUAGUGCUGCCAGAAAAAGACAGCUGGACUGUCAAUGACAUACAGAAGU |
| | | UAGUGGGAAAAUUAAAUUGGGCAAGUCAAAUUUAUGCAGGGAUUAAAGUA |
| | | AAGCAAUUAUGUAAACUCCUUAGGGGAACCAAAGCACUUACAGAAGUAAU |
| | | ACCACUAACAAAAGAAGCAGAGCUAGAACUGGCAGAAAACAGGGAGAUUU |
| | | UAAAGGAACCAGUACAUGGAGUGUAUUAUGACCCAUCAAAAGACUUAAUA |
| | | GUAGAAAUACAGAAGCAGGGGCAAGGCCAAUGGACAUAUCAAAUUUUUCA |
| | | AGAGCCAUUUAAAAAUCUGAAAACAGGAAAAUAUGCAAAAACGAGGAGUG |
| | | CCCACACUAAUGAUGUAAAACAAUUAACAGAGGCAGUGCAAAAAAUAGCC |
| | | AAUGAAAGCAUAGUAAUAUGGGGAAAGAUUCCUAAAUUUAAAUUACCCAU |
| | | ACAAAAAGAAACAUGGGAAACAUGGUGGACAGAGUAUUGGCAAGCCACCU |
| | | GGAUUCCUGAGUGGGAGUUUGUCAAUACCCCUCCCUUAGUGAAAUUAUGG |
| | | UACCAGUUAGAAAAAGAACCCAUAGUAGGAGCAGAAACUUUCUAUGUAGA |
| | | UGGGGCAGCUAACAGGGAGACUAAAUUAGGAAAAGCAGGAUAUGUUACUA |
| | | GCAGAGGAAGGCAAAAAGUUGUCUCCCUAACAGACACAACAAAUCAGAAA |
| | | ACUGAGUUACAAGCAAUUCACCUAGCUUUGCAGGAUUCAGGAUUAGAAGU |
| | | AAACAUAGUAACAGACUCACAAUAUGCAUUAGGAAUCAUUCAAGCACAAC |
| | | CAGAUAAAAGUGAAUCAGAGUUAGUCAGUCAAAUAAUAGAACAGCUAAUA |
| | | AAAAAGGAAAAAGUCUACCUGGCAUGGGUACCAGCACACAAAGGAAUUGG |
| | | AGGAAAUGAACAGGUAGAUAAAUUAGUCAGUGCUGGAAUCAGGAGAGUAC |
| | | UAUUUCUAGAUGGAAUAGAGAAGGCCCAAGAAGAACAUGAGAAAUAUCAU |
| | | AAUAAUUGGAGAGCAAUGGCUAGUGAAUUUAACCUGCCAGCUGUAGUAGC |
| | | AAAAGAGAUAGUAGCCUGCUGUGAUAAGUGCCAGGUAAAAGGAGAAGCCA |
| | | UGCAUGGACAAGUAGACUGCAGUCCAGGAAUAUGGCAACUAGAUUGUACA |
| | | CAUUUAGAAGGAAAAGUUAUCCUGGUAGCAGUUCAUGUAGCCAGUGGAUA |
| | | UAUAGAAGCAGAGGUUAUUCCAGCAGAGACAGGACAGGAAACAGCAUACU |
| | | UUAUUUUAAAAUUAGCAGGAAGAUGGCCAGUAAAAACAAUACAUACAGAC |
| | | AAUGGCAGUAAUUUCACCAGUACUACGGUUAAGGCCGCCUGUUGGUGGGC |
| | | AGGGAUCAAGCAGGAAUUUGGCAUUCCCUACAAUCCCCAAAGUCAAGGAG |
| | | UAGUAGAAUCUAUGAAUAAAGAAUUAAAGAGAAUUAUAGAACAAGUAAGA |
| | | GAUCAGGCUGAACAUCUUAAGACAGCAGUACAAAUGGCAGUAUUCAUUCA |
| | | CAAUUUUAAAAGAAAAGGGGGGAUUGGGGGGUACAGUGCAGGGGAAAGAA |
| | | UAGUAGACAUAAUAGCAUCAGACAUACAAACUAAAGAACUACAAAAACAA |
| | | AUUACAAAAAUUCAAAAUUUUCGGGUUUAUUACAGGGACAGCAGAGAUCC |
| | | ACUUUGGAAAGGACCAGCAAAGCUUCUUUGGAAAGGUGAAGGGGCAGUAG |
| | | UAAUACAAGAUAAGAGUGACAUAAAAGUAGUGCCAAGAAGAAAAGCAAAG |
| | | AUUAUCAGGGAUUAUGGAAAACAGAUGGCAGGUGAUGAUUGUGUGGCAAG |
| | | UAGACAGGAUGAGGAUUAGAACAUGGAAAAGUUUAGUAAAACACCAUAUG |
| | | UAUGUUUCAAAGAAAGCUAAGGGAUGGUUUUAUAGACAUCACUAUGAAAG |
| | | CACUCAUCCAAGAAUAAGUUCAGAAGUACAUAUCCCACUAGGGGAUGCUA |
| | | GCUUGGUAGUAACAACAUAUUGGGGUCUACAUACAGGAGAAAGAGACUGG |
| | | CAUUUGGGUCAGGGAGUCUCCAUAGAAUGGAGGAAAAGGAGAUACAGCAC |
| | | ACAAGUAGACCCUGACCUAGCAGACCAACUAAUUCAUCUGUACUACUUUG |
| | | AUUGUUUUUCAGAAUCUGCUAUAAGAAAUGCCAUAUUAGGACAUAGAGUU |
| | | AGUCCUAGGUGUGAAUAUCAAGCAGGACAUAACAAGGUAGGAUCUCUACA |
| | | GUACUUGGCACUAGCAGCAUUAGUAACACCAAGAAAGAUAAAGCCACCUU |
| | | UGCCUAGUGUUGCGAAACUGACAGAGGACAGAUGGAACAAGUCCCACAAG |
| | | ACCAAGGGCCACAGAGGGAGCCAUACAAUGAAUGGACACUAAAGCUUUUA |
| | | GAGGAGCUUAAGAAUGAAGCUGUCAGACAUUUCCCUAGACCAUGGCUUCA |
| | | UGGCCUAGGGCAAUAUAUCUAUGAAACUUAUGAGGAUACUUGGGCAGGAG |
| | | UGGAAGCCAUAAUAAGAAUUCUGCAACAAUUGCUGCUUAUUCAUUUCAGA |
| | | AUUGGGUGUCAACAUAGCAGAAUAGGCAUUAUUCGACAGAGGAGAACAAG |
| | | AAAUGGAGCCAGUAGAUCCUAGACUAGAGCCCUGGAAGCAUCCAGGAAGU |
| | | CAGCCUAAGACUGCCUGUACCAAUUGCUAUUGCAAAAAGUGUUGCUUGCA |
| | | UUGCCAAGUUUGCUUCAUAACAAAAGGCUUAGGCAUCUCCUAUGGCAGGA |
| | | AGAAGCGGAAAAAGCGACGAAGAUCUCCUCAACACAGUCAGACUGAUCAG |
| | | GCUUCUCUAUCAAAGCAGUAAGUAGUACAUGUAAUGCAACCUUUAGUAAU |
| | | AUUAGCAAUAGUAGCAUUAGUAGUAGCACUAAUAAUAGUCAUAGUUGUAU |
| | | GGUCCAUUGUAUUAAUAGAAUAUAGAAAAAUAUUAAGACAAAAGAAAAUA |
| | | GACAGGUUAAUUGAUAGAAUAAGAGAAAAAGCAGAAGACAGUGGCAAUGA |
| | | GAGUGAUGGGGAUCAGGAAGAAUUAUCAGCACUUGUGGAAAGGGGGCACC |
| | | UUGCUCCUUGGGAUAUUGAUGAUCUGUAGUGCUGCAGAACAAUUGUGGGU |
| | | CACAGUCUAUUAUGGGGUACCUGUGUGGAAAGAAGCAAACACCACUCUAU |
| | | UUUGUGCAUCAGAUGCUAAGGCAUAUGAUACAGAGGUACAUAAUGUUUGG |
| | | GCCACACAUGCCUGUGUACCCACAGACCCCAACCCACAAGAAAUACUAUU |
| | | GGAAAAUGUGACAGAAGAUUUUAACAUGUGGAAAAAUAACAUGGUAGAAC |
| | | AGAUGCAUGAGGAUAUAAUCAGUUUAUGGGAUCAAAGUCUAAAGCCAUGU |
| | | GUAAAAUUAACCCCACUCUGUGUUACUUUACAUUGCACUGAUUUGAAGAA |
| | | UGGUACUAAUUUGAAGAAUGGUACUAAUUUGAAGAAUGGUACUAAAAUCA |
| | | UUGGGAAAUCAAUAAGAGGAGAAAUAAAAAACUGCUCUUUCAAUGUCACC |
| | | AAAAACAUAAUAGAUAAGGUGAAAAAAGAAUAUGCGCUUUUCUAUAGACA |
| | | UGAUGUAGUACCAAUAGAUAGGAAUAUUACUAGCUAUAGGUUAAUAAGUU |
| | | GUAACACCUCAACCCUUACACAGGCCUGUCCAAAGGUAUCCUUUGAGCCA |
| | | AUUCCCAUACAUUAUUGUGCCCCGGCUGGUUUUGCGAUUCUAAAAUGUAA |
| | | AGAUAAGAAGUUCAAUGGAACGGGACCAUGUACAAAUGUCAGUACAGUAC |
| | | AAUGUACACAUGGAAUUAGGCCAGUAGUAUCAACUCAACUGCUGUUAAAU |
| | | GGAAGUCUAGCAGAGGAAGAGGUAGUAAUUAGAUCUAGCAAUUUCACGGA |
| | | CAAUGCUAAAAUCAUAAUAGUACAGCUGAAUGAAACUGUAGAAAUUAAUU |
| | | GUACAAGACCCAACAACAAUACAAGAAAAGGGAUAACUCUAGGACCAGGG |
| | | AGAGUAUUUUAUACAACAGGAAAAAUAGUAGGAGAUAUAAGAAAAGCACA |
| | | UUGUAACAUUAGUAAAGUAAAAUGGCAUAACACUUUAAAAAGGGUAGUUA |
| | | AAAAAUUAAGAGAAAAAUUUGAAAAUAAAACAAUAAUCUUUAAUAAAUCC |
| | | UCAGGGGGGGACCCAGAAAUUGUAAUGCACAGCUUUAAUUGUGGAGGGGA |
| | | AUUUUUCUACUGUAAUACAAAAAAACUGUUUAAUAGUACUUGGAAUGGUA |
| | | CUGAAGGGUCAUAUAACAUUGAAGGAAAUGACACUAUCACACUCCCAUGC |
| | | AGAAUAAAACAAAUUAUAAACAUGUGGCAGGAAGUAGGAAAAGCAAUGUA |
| | | UGCCCCUCCCAUCAGUGGACAAAUUUGGUGCUCAUCAAAUAUUACAGGGC |
| | | UGCUACUAACAAGAGAUGGUGGUAAGAACAGCAGCACCGAAAUCUUCAGA |
| | | CCUGGAGGAGGAGAUAUAAGGGACAAUUGGAGAAGUGAAUUAUAUAAAUA |
| | | UAAAGUAGUAAGAGUUGAACCAUUAGGAAUAGCACCCACCAAGGCAAAAA |
| | | GAAGAGUGGUGCAGAGAGAAAAAAGAGCAGUGGGAAUAGGAGCUGUGUUC |
| | | CUUGGGUUCUUGGGAGCAGCAGGAAGCACUAUGGGCGCAGCGUCAAUAAC |
| | | GCUGACGGUACAGGCCAGACAAUUAUUGUCCGGUAUAGUGCAACAGCAGA |
| | | ACAAUUUGCUGAGGGCUAUUGAAGCGCAACAGCAUAUGUUGCAACUCACA |
| | | GUCUGGGGCAUCAAGCAGCUCCAGGCAAGAGUCCUGGCUGUGGAAAGAUA |
| | | CCUACAGGAUCAACAGCUCCUGGGGAUUUGGGGUUGCUCUGGAAAACUCA |
| | | UCUGCACCACUACUGUGCCUUGGAAUACUAGUUGGAGUAAUAAAUCUCUG |
| | | GAUACAAUUUGGGGUAACAUGACCUGGAUGCAGUGGGAAAAAGAAAUUAA |
| | | CAAUUACACAGGCUUAAUAUACAACUUGAUUGAAGAAUCGCAGAACCAAC |
| | | AAGAAAAGAAUGAACAAGAAUUAUUGGCAUUAGAUAAAUGGGCAAGUUUG |
| | | UGGAAUUGGUUUAACAUAUCAAACUGGCUGUGGUAUAUAAAAAUAUUCAU |
| | | AAUGAUAGUAGGAGGCUUGAUAGGUUUAAGAAUAGUUUUCAGUGUACUUU |
| | | CUAUAGUGAAUAGAGUUAGGCAGGGAUACUCACCAUUAUCGUUUCAGACC |
| | | CGCUUCCCAGCCUCGAGGGGACCCGACAGGCCCGAAGGAAUCGAAGAAGA |
| | | AGGUGGAGACAGAGACAGAGACAGAUCCAGUCCAUUAGUGGAUGGAUUCU |
| | | UAGCAAUCAUCUGGGUCGACCUGCGGAGCCUGUUCCUCUUCAGCUACCAC |
| | | CGCUUGAGAGACUUACUCUUGAUUGUAACGAGGAUUGUGGAACUUCUGGG |
| | | ACGCAGGGGGUGGGAACUCCUCAAAUACUUGUGGAACCUCCUGCAGUAUU |
| | | GGGGUCAGGAACUAAAGAAUAGUGCUGUUAGCUUGCUUAACGCCACAGCC |
| | | AUAGCAGUAGGUGAGGGAACAGAUAGAAUUAUAGAAAUAUUACAAAGAGC |
| | | UGGUAGAGCUAUUCUCAACAUACCUACGAGAAUAAGACAGGGCUUAGAAA |
| | | GGGCUUUGCUAUAAGCUUAUGGGUGGAGCUAUUUCCAUGAGGCGGUCCAG |
| | | GCCGUCUGGAAAUCUGUACGAGAGACUCUUGCGGGCGCGUGGGGAGACUU |
| | | AUGGAAAACUCUUAGGAGAGGUAAAAGAUGGAUACUUGCAAUCCCCAGGA |
| | | GGAUUAGACAAGGGCUUGAGCUCACUCUCUUGUGAGGGACAAAAAUACAA |
| | | UCAGGGACAGUAUAUGAAUACUCCAUGGAGAAACCCAGCUAAAGAGAGAG |
| | | AAAAAUUAGCAUACAGAAAACAAAAUAUGGAUGAUAUAGAUAAGGAGGAU |
| | | GAUGACUUGGUAGGGGUAUCAGUGAGGCCAAAAGUUCCCCUAAGAACAAU |
| | | GAGUUACAAAGUGGCAAUAGACAUGUCUCAUUUUAUAAAAGAAAAGGGGG |
| | | GACUGGAAGGGAUUUAUUACAGUGCAAGAAGACAUAGAAUCUUAGACAUA |
| | | UACUUAGAAAAGGAAGAAGGCAUCAUACCAGAUUGGCAGGAUUACACCUC |
| | | AGGACCAGGAAUUAGAUACCCAAAGACAUUUGGCUGGCUAUGGAAAUUAG |
| | | UCCCUGUAAAUGUAUCAGAUGAGGCACAGGAGGAUGAGGAGCAUUAUUUA |
| | | AUGCAUCCAGCUCAAACUUCCCAGUGGGAUGACCCUUGGGGAGAGGUUCU |
| | | AGCAUGGAAGUUUGAUCCAACUCUGGCCUACACUUAUGAGGCGUAUGUUA |
| | | GAUACCCAGAAGAGUUUGGAAGCAAGUCAGGCCUGUCAGAGGAAGAGGUU |
| | | AAAAGAAGGCUAACCGCAAGAGGCCUUCUUAACAUGGCUGACAAGAAGGA |
| | | AACUCGCUGAAUUCGAGCUAUCUACAGGGGACUUUCCGCUGGGGACUUUC |
| | | CAGGGAGGCGUGGCCUGGGCGGGACCGGGGAGUGGCGAGCCCUCAGAUGC |
| | | UGCAUAUAAGCAGCCGCUUUUGCCUGUACUGGGUCUCUCUAGUUAGACCA |
| | | GAUCUGAGCCUGGGAGCUCUCUGGCUAGCUGAGAACCCACUGCUUAAGCC |
| | | UCAAUAAAGCUUGCCUUGAGUGCUUUAAAUAGUGUGUGCCCGUCUGUUGU |
| | | GUGACUCUGGUAACUAGAGAUCCCUCAGACCAUUUUAGUCAGUGUGGAAA |
| | | AUCUCUAGCA |
| SEQ ID | clone | UGGAAGGGAUUUAUUACAGUGCAAGAAGACAUAGAAUCUUAGACAUAUAC |
| NO: 54 | P10.21 RNA | UUAGAAAAGGAAGAAGGCAUCAUACCAGAUUGGCAGGAUUACACCUCAGG |
| | | ACCAGGAAUUAGAUACCCAAAGACAUUUGGCUGGCUAUGGAGAUUAGUCC |
| | | CUGUAAAUGUAUCAGAUGAGGCACAGGAGGAUGAGGAGCAUUAUUUAAUG |
| | | CAUCCAGCUCAAACUUCCCAGUGGGAUGACCCUUGGGGAGAGGUUCUAGC |
| | | AUGGAAGUUUGAUCCAACUCUGGCCUACACUUAUGAGGCAUAUGUUAGAU |
| | | ACCCAGAAGAGUUUGGAAGCAAGUCAGGCCUGUCAGAGGAAGAGGUUAAA |
| | | AGAAGGCUAACCGCAAGAGGCCUUCUUAACAUGGCUGACAAGAAGGAAAC |
| | | UCGCUGAAUUCGAGCUAUCUACAGGGGACUUUCCGCUGGGGACUUUCCAG |
| | | GGAGGCGUGGCCUGGGCGGGACCGGGGAGUGGCGAGCCCUCAGAUGCUGC |
| | | AUAUAAGCAGCCGCUUUUGUCUGUACUGGGUCUCUCUAGUUAGACCAGAU |
| | | CUGAGCCUGGGAGCUCUCUGGCUAGCUGAGAACCCACUGCUUAAGCCUCA |
| | | AUAAAGCUUGCCUUGAGUGCUUUAAGUAGUGUGUGCCCGUCUGUUGUGUG |
| | | ACUCUGGUAACUAGAGAUCCCUCAGACCAUUUUAGUCAGUGUGGAAAAUC |
| | | UCUAGCAGUGGCGCCCGAACAGGGACCGGAAAGCGAAAGAGAAACCAGAG |
| | | AAGCUCUCUCGACGCAGGACUCGGCUUGCUGAAGCGCGCACGGCAAGCGG |
| | | CGAGGGGCAGCGACCGGUGAGUACGCUAAAAAUUUUGACUAGCGGAGGCU |
| | | AGAAGGAGAGAGAUGGGUGCGAGAGCGUCAGUAUUAAGCGGGGGAAAAUU |
| | | GGAUGCAUGGGAAAAAAUUCGGUUACGGCCAGGAGGAAAGAAAAAAUAUA |
| | | GACUAAAACAUCUAGUAUGGGCAAGCAGGGAGCUAGAACGAUUUGCACUU |
| | | AAUCCUGGCCUUUUAGAGACAUCAGAUGGCUGUAAACAAAUAAUAGGACA |
| | | GCUACAACCAGCUAUCCGGACAGGAUCAGAAGAAUUUAGAUCAUUAUUUA |
| | | AUACAGUAGCAACCCUCUAUUGUGUACAUGAAAGGAUAGAGGUAAAAGAC |
| | | ACCAAGGAAGCUUUAGAGAAGAUAGAGGAAGAGCAAAACAAAAGUAAGAA |
| | | AAAAGCACAGCAAGCAGCAGCUGACACAGGACACAGCAAUCAGGUCAGCC |
| | | AAAAUUACCCUAUAGUGCAGAACAUCCAGGGGCAAAUGGUACAUCAGGCC |
| | | CUAUCACCUAGAACUUUAAAUGCGUGGGUAAAAGUAGUAGAAGAGAAGGC |
| | | UUUUAGCCCAGAAGUAAUACCCAUGUUUUCAGCAUUAUCAGAAGGAGCCA |
| | | CCCCACAAGAUUUAAACACCAUGCUAAACACAGUGGGGGGACAUCAAGCA |
| | | GCCAUGCAAAUGUUAAAAGAGACCAUCAAUGAGGAAGCUGCAGAAUGGGA |
| | | UAGAGUGCAUCCAGUGCAUGCAGGGCCUAUUGCACCAGGCCAGAUGAGAG |
| | | AACCAAGGGGAAGUGACAUAGCAGGAACUACUAGUACCCUUCAGGAACAA |
| | | AUAGGAUGGAUGACACAUAAUCCACCUAUCCCAGUAGGAGAAAUCUAUAA |
| | | AAGAUGGAUAAUCCUGGGAUUAAAUAAAAUAGUAAGAAUGUAUAGCCCUA |
| | | CCAGCAUUCUGGACAUAAGACAAGGACCAAAGGAACCCUUUAGAGACUAU |
| | | GUAGACCGGUUCUAUAAAACCCUAAGAGCCGAGCAAGCUACACAGGAGGU |
| | | AAAAAAUUGGAUGACAGAAACCUUGUUGGUCCAAAAUGCGAACCCAGAUU |
| | | GUAAAACUAUUUUAAAAGCAUUGGGACCAGCAGCCACACUAGAAGAAAUG |
| | | AUGACAGCAUGUCAGGGAGUGGGAGGACCCGGCCAUAAAGCAAGAGUUUU |
| | | GGCUGAAGCAAUGAGCCAAGUAACAAAUUCAGCUACCAUAAUGAUGCAGA |
| | | GAGGCAAUUUUAGGAACCAAAGAAAAACUGUUAAGUGUUUCAAUUGUGGC |
| | | AAAGAAGGGCACAUAGCCAAAAAUUGCAGGGCUUCUAGGAAAAAGGGCUG |
| | | UUGGAAAUGUGGAAAGGAAGGACACCAAAUGAAAGAUUGUACUGAGAGAC |
| | | AGGCUAAUUUUUUAGGGAAGAUCUGGCCUUCCCACAAGGGAAGGCCAGGA |
| | | AAUUUUCUUCAGAGCAGACCAGAGCCAACAGCCCCAUCAGAAGAGAGCGU |
| | | CAAGUUUGGAGAAGAGACAACAACUCCCUCUCAGAAGCAGGAGCCGAUAG |
| | | ACAAGGAACUGUAUCCUUUAACUUCCCUCAGAUCACUCUUUGGCAACGAC |
| | | CCCUCGUCACAAUAAAGAUAGGGGGGCAACUAAAGGAAGCUCUAUUAGAU |
| | | ACAGGAGCAGAUGAUACAGUAUUAGAAGACAUGGAUUUGCCAGGAAGAUG |
| | | GAAACCAAAAAUGAUAGGGGGAAUUGGAGGUUUUAUCAAAGUAAGACAGU |
| | | AUGAUCAGAUACCCAUAGAUAUCUGUGGACAUAAAGCUGUAGGUACAGUA |
| | | UUAGUAGGACCUACACCUGUCAACAUAAUUGGAAGAAAUCUGUUGACUCA |
| | | GAUUGGUUGCACUUUAAAUUUUCCCAUUAGUCCUAUUGAAACUGUACCAG |
| | | UAAAAUUAAAGCCAGGAAUGGAUGGCCCAAAAGUCAAACAAUGGCCAUUG |
| | | ACAGAAGAAAAAAUAAAAGCAUUAGUAGAAAUUUGUACAGAAAUGGAAAA |
| | | GGAAGGAAAGAUUUCAAAAAUUGGGCCUGAAAAUCCAUACAAUACUCCAG |
| | | UAUUUGCCAUAAAGAAAAAAGACAGUACUAAAUGGAGAAAAUUAGUAGAU |
| | | UUCAGAGAACUUAAUAGGAAAACUCAAGACUUCUGGGAAGUUCAAUUAGG |
| | | AAUACCACAUCCCGCAGGGUUAAAAAAGAAAAAAUCAGUAACAGUACUGG |
| | | AUGUGGGUGAUGCAUAUUUUUCAGUUCCCUUAGAUAAAGACUUCAGGAAG |
| | | UAUACUGCAUUUACCAUACCUAGUAUAAACAAUGAGACACCAGGGAUUAG |
| | | AUAUCAGUACAAUGUGCUUCCACAGGGAUGGAAAGGAUCACCAGCAAUAU |
| | | UCCAAAGUAGCAUGACAAAAACCUUAGAGCCUUUUAGAAAACAAAAUCCA |
| | | GACAUAAUUAUCUAUCAAUACAUGGAUGAUUUGUAUGUAGGAUCUGACUU |
| | | AGAAAUAGGGCAGCAUAGAACAAAAAUAGAGGAACUGAGACAACAUCUGU |
| | | UAAAGUGGGGAUUUACCACACCAGACAAAAAACAUCAGAAAGAACCUCCA |
| | | UUCCUUUGGAUGGGUUAUGAACUCCAUCCUGAUAAAUGGACAGUACAGCC |
| | | UAUAGUGCUGCCAGAAAAAGACAGCUGGACUGUCAAUGACAUACAGAAGU |
| | | UAGUGGGAAAAUUAAAUUGGGCAAGUCAAAUUUAUGCAGGGAUUAAAGUA |
| | | AAGCAAUUAUGUAAACUCCUUAGGGGAACCAAAGCACUUACAGAAGUAAU |
| | | ACCACUAACAAAAGAAGCAGAGCUAGAACUGGCAGAAAACAGGGAGAUUU |
| | | UAAAGGAACCAGUACAUGGAGUGUAUUAUGACCCAUCAAAAGACUUAAUA |
| | | GUAGAAAUACAGAAGCAGGGGCAAGGCCAAUGGACAUAUCAAAUUUUUCA |
| | | AGAGCCAUUUAAAAAUCUGAAAACAGGAAAAUAUGCAAAAACGAGGGGUG |
| | | CCCACACUAAUGAUGUAAAACAAUUAACAGAGGCAGUGCAAAAAAUAGCC |
| | | AAUGAAAGCAUAGUAAUAUGGGGAAAGAUUCCUAAAUUUAAAUUACCCAU |
| | | ACAAAAAGAAACAUGGGAAACAUGGUGGACAGAGUAUUGGCAAGCCACCU |
| | | GGAUUCCUGAGUGGGAGUUUGUCAAUACCCCUCCCUUAGUGAAAUUAUGG |
| | | UACCAGUUAGAAAAAGAACCCAUAGUAGGAGCAGAAACUUUCUAUGUAGA |
| | | UGGGGCAGCUAACAGGGAGACUAAAUUAGGAAAAGCAGGAUAUGUUACUA |
| | | GCAGAGGAAGGCAAAAAGUUGUCUCCCUAACAGACACAACAAAUCAGAAA |
| | | ACUGAGUUACAAGCAAUUCACCUAGCUUUGCAGGAUUCAGGAUUAGAAGU |
| | | AAACAUAGUAACAGACUCACAAUAUGCAUUAGGAAUCAUUCAAGCACAAC |
| | | CAGAUAAAAGUGAAUCAGAGUUAGUCAGUCAAAUAAUAGAACAGCUAAUA |
| | | AAAAAGGAAAAAGUCUACCUGGCAUGGGUACCAGCACACAAAGGAAUUGG |
| | | AGGAAAUGAACAGGUAGAUAAAUUAGUCAGUGCUGGAAUCAGGAGAGUAC |
| | | UAUUUCUAGAUGGAAUAGAGAAGGCCCAAGAAGAACAUGAGAAAUAUCAU |
| | | AAUAAUUGGAGAGCAAUGGCUAGUGAAUUUAACCUGCCAGCUGUAGUAGC |
| | | AAAAGAGAUAGUAGCCUGCUGUGAUAAGUGCCAGGUAAAAGGAGAAGCCA |
| | | UGCAUGGACAAGUAGACUGCAGUCCAGGAAUAUGGCAACUAGAUUGUACA |
| | | CAUUUAGAAGGAAAAGUUAUCCUGGUAGCAGUUCAUGUAGCCAGUGGAUA |
| | | UAUAGAAGCAGAGGUUAUUCCAGCAGAGACAGGACAGGAAACAGCAUACU |
| | | UUAUUUUAAAAUUAGCAGGAAGAUGGCCAGUAAAAACAAUACAUACAGAC |
| | | AAUGGCAGUAAUUUCACCAGUACUACGGUUAAGGCCGCCUGUUGGUGGGC |
| | | AGGGAUCAAGCAGGAAUUUGGCAUUCCCUACAAUCCCCAAAGUCAAGGAG |
| | | UAGUAGAAUCUAUGAAUAAAGAAUUAAAGAAAAUUAUAGAACAAGUAAGA |
| | | GAUCAGGCUGAACAUCUUAAGACAGCAGUACAAAUGGCAGUAUUCAUUCA |
| | | CAAUUUUAAAAGAAAAGGGGGGAUUGGGGGGUACAGUGCAGGGGAAAGAA |
| | | UAGUAGACAUAAUAGCAUCAGACAUACAAACUAAAGAACUACAAAAACAA |
| | | AUUACAAAAAUUCAAAAUUUUCGGGUUUAUUACAGGGACAGCAGAGAUCC |
| | | ACUUUGGAAAGGACCAGCAAAGCUUCUUUGGAAAGGUGAAGGGGCAGUAG |
| | | UAAUACAAGAUAAGAGUGACAUAAAAGUAGUGCCAAGAAGAAAAGCAAAG |
| | | AUUAUCAGGGAUUAUGGAAAACAGAUGGCAGGUGAUGAUUGUGUGGCAAG |
| | | UAGACAGGAUGAGGAUUAGAACAUGGAAAAGUUUAGUAAAACACCAUAUG |
| | | UAUGUUUCAAAGAAAGCUAAGGGAUGGUUUUAUAGACAUCACUAUGAAAG |
| | | CACUCAUCCAAGAAUAAGUUCAGAAGUACAUAUCCCACUAGGGGAUGCUA |
| | | GCUUGGUAGUAACAACAUAUUGGGGUCUACAUACAGGAGAAAGAGACUGG |
| | | CAUUUGGGUCAGGGAGUCUCCAUAGAAUGGAGGAAAAGGAGAUACAGCAC |
| | | ACAAGUAGACCCUGACCUAGCAGACCAACUAACUCAUCUGUACUACUUUG |
| | | AUUGUUUUUCAGAAUCUGCUAUAAGAAAUGCCAUAUUAGGACAUAGAGUU |
| | | AGUCCUAGGUGUGAAUAUCAAGCAGGACAUAACAAGGUAGGAUCUCUACA |
| | | GUACUUGGCACUAGCAGCAUUAGUAACACCAAGAAAGAUAAAGCCACCUU |
| | | UGCCUAGUGUUGCGAAACUGACAGAGGACAGAUGGAACAAGUCCCACAAG |
| | | ACCAAGGGCCACAGAGGGAGCCAUACAAUGAAUGGACACUAAAGCUUUUA |
| | | GAGGAGCUUAAGAAUGAAGCUGUCAGACAUUUCCCUAGACCAUGGCUUCA |
| | | UGGCCUAGGGCAAUAUAUCUAUGAAACUUAUGAGGAUACUUGGGCAGGAG |
| | | UGGAAGCCAUAAUAAGAAUUCUGCAACAAUUGCUGCUUAUUCAUUUCAGA |
| | | AUUGGGUGUCAACAUAGCAGAAUAGGCAUUAUUCGACAGAGGAGAACAAG |
| | | AAAUGGAGCCAGUAGAUCCUAGACUAGAGCCCUGGAAGCAUCCAGGAAGU |
| | | CAGCCUAAGACUGCCUGUACCAAUUGCUAUUGCAAAAAGUGUUGCUUGCA |
| | | UUGCCAAGUUUGCUUCAUAACAAAAGGCUUAGGCAUCUCCUAUGGCAGGA |
| | | AGAAGCGGAAAAAGCGACGAAGAUCUCCUCAACACAGUCAGACUGAUCAA |
| | | GCUUCUCUAUCAAAGCAGUAAGUAGUACAUGUAAUGCAACCUUUAGUAAU |
| | | AUUAGCAAUAGUAGCAUUAGUAGUAGCACUAAUAAUAGUCAUAGUUGUAU |
| | | GGUCCAUUGUAUUAAUAGAAUAUAGAAAAAUAUUAAGACAAAAGAAAAUA |
| | | GACAGGUUAAUUGAUAGAAUAAGAGAAAAAGCAGAAGACAGUGGCAAUGA |
| | | GAGUGAUGGGGAUCAGGAAGAAUUAUCAGCACUUGUGGAAAGGCGGCACC |
| | | UUGCUCCUUGGGAUAUUGAUGAUCUGUAGUGCUGCAGAACAAUUGUGGGU |
| | | CACAGUCUAUUAUGGGGUACCUGUGUGGAAAGAAGCAAACACCACUCUAU |
| | | UUUGUGCAUCAGAUGCUAAAGCAUAUGAUACAGAGGUACAUAAUGUUUGG |
| | | GCCACACAUGCCUGUGUACCCACAGACCCCAACCCACAAGAAAUACUAUU |
| | | GGAAAAUGUGACAGAAGAUUUUAACAUGUGGAAAAAUAACAUGGUAGAAC |
| | | AGAUGCAUGAGGAUAUAAUCAGUUUAUGGGAUCAAAGUCUAAAGCCAUGU |
| | | GUAAAAUUAACCCCACUCUGUGUUACUUUACAUUGCACUGAUUUGAAGAA |
| | | UGGUACUAAUUUGAAGAAUGGUACUAAAAUCAUUGGGAAAUCAAUAAGAG |
| | | GAGAAAUAAAAAACUGCUCUUUCAAUGUCACCAAAAACAUAAUAGAUAAG |
| | | GUGAAAAAAGAAUAUGCGCUUUUCUAUAGACAUGAUGUAGUACCAAUAGA |
| | | UAGGAAUAUUACUAGCUAUAGGUUAAUAAGUUGUAACACCUCAACCCUUA |
| | | CACAGGCCUGUCCAAAGGUAUCCUUUGAGCCAAUUCCCAUACAUUAUUGU |
| | | GCCCCGGCUGGUUUUGCGAUUCUAAAAUGUAAAGAUAAGAAGUUCAAUGG |
| | | AACGGGACCAUGUACAAAUGUCAGUACAGUACAAUGUACACAUGGAAUUA |
| | | GGCCAGUAGUAUCAACUCAACUGCUGUUAAAUGGAAGUCUAGCAGAAGAA |
| | | GAGGUAGUAAUUAGAUCUAGCAAUUUCACGGACAAUGCUAAAAUCAUAAU |
| | | AGUACAGCUGAAUGAAACUGUAGAAAUUAAUUGUACAAGACCCAACAACA |
| | | AUACAAGAAAAGGGAUAACUCUAGGACCAGGGAGAGUAUUUUAUACAACA |
| | | GGAAAAAUAGUAGGAGAUAUAAGAAAAGCACAUUGUAACAUUAGUAAAGU |
| | | AAAAUGGCAUAACACUUUAAAAAGGGUAGUUAAAAAAUUAAGAGAAAAAU |
| | | UUGAAAAUAAAACAAUAAUCUUUAAUAAAUCCUCAGGGGGGGACCCAGAA |
| | | AUUGUAAUGCACAGCUUUAAUUGUGGAGGGGAAUUUUUCUACUGUAAUAC |
| | | AAAAAAACUGUUUAAUAGUACUUGGAAUGGUACUGAAGGGUCAUAUAACA |
| | | UUGAAGGAAAUGACACUAUCACACUCCCAUGCAGAAUAAAACAAAUUAUA |
| | | AACAUGUGGCAGGAAGUAGGAAAAGCAAUGUAUGCCCCUCCCAUCAGUGG |
| | | ACAAAUUUGGUGCUCAUCAAAUAUUACAGGGCUGCUACUAACAAGAGAUG |
| | | GUGGUAAGAACAGCAGCACCGAAAUCUUCAGACCUGGAGGAGGAGAUAUA |
| | | AGGGACAAUUGGAGAAGUGAAUUAUAUAAAUAUAAAGUAGUAAGAGUUGA |
| | | ACCAUUAGGAAUAGCACCCACCAAGGCAAAAAGAAGAGUGGUGCAGAGAG |
| | | AAAAAAGAGCAGUAGGAAUAGGAGCUGUGUUCCUUGGGUUCUUGGGAGCA |
| | | GCAGGAAGCACUAUGGGCGCAGCGUCAAUAACGCUGACGGUACAGGCCAG |
| | | ACAAUUAUUGUCUGGUAUAGUGCAACAGCAGAACAAUUUGCUGAGGGCUA |
| | | UUGAAGCGCAACAGCAUAUGUUGCAACUCACAGUCUGGGGCAUCAAGCAG |
| | | CUCCAGGCAAGAGUCCUGGCUGUGGAAAGAUACCUACAGGAUCAACAGCU |
| | | CCUGGGGAUUUGGGGUUGCUCUGGAAAACUCAUCUGCACCACUACUGUGC |
| | | CUUGGAAUACUAGUUGGAGUAAUAAAUCUCUGGAUACAAUUUGGGGUAAC |
| | | AUGACCUGGAUGCAGUGGGAAAAAGAAAUUAACAAUUACACAGGCUUAAU |
| | | AUACAACUUGAUUGAAGAAUCGCAGAACCAACAAGAAAAGAAUGAACAAG |
| | | AAUUAUUGGCAUUAGAUAAAUGGGCAAGUUUGUGGAAUUGGUUUAACAUA |
| | | UCAAACUGGCUGUGGUAUAUAAAAAUAUUCAUAAUGAUAGUAGGAGGCUU |
| | | GAUAGGUUUAAGAAUAGUUUUCAGUGUACUUUCUAUAGUGAAUAGAGUUA |
| | | GGCAGGGAUACUCACCAUUAUCGUUUCAGACCCGCUUCCCAGCCUCGAGG |
| | | GGACCCGACAGGCCCGAAGGAAUCGAAGAAGAAGGUGGAGACAGAGACAG |
| | | AGACAGAUCCAGUCCAUUAGUGGAUGGAUUCUUAGCAAUCAUCUGGGUCG |
| | | ACCUGCGGAGCCUGUUCCUCUUCAGCUACCACCGCUUGAGAGACUUACUC |
| | | UUGAUUGUAACGAGGAUUGUGGAACUUCUGGGACGCAGGGGGUGGGAACU |
| | | CCUCAAAUACUUGUGGAAUCUCCUGCAGUAUUGGAGUCAGGAACUAAAGA |
| | | AUAGUGCUGUUAGCUUGCUUAACGCCACAGCCAUAGCAGUAGGUGAGGGA |
| | | ACAGAUAGAAUUAUAGAAAUAUUACAAAGAGCUGGUAGAGCUAUUCUCAA |
| | | CAUACCUACGAGAAUAAGACAGGGCUUAGAAAGGGCUUUGCUAUAAGCUU |
| | | AUGGGUGGAGCUAUUUCCAUGAGGCGGUCCAGGCCGUCUGGAAAUCUGUA |
| | | CGAAAGACUCUUGCGGGCGCGUGGGGAGACUUAUGGAAAACUCUUAGGAG |
| | | AGGUAAAAGAUGGAUACUCGCAAUCCCCAGGAGGAUUAGACAAGGGCUUG |
| | | AGCUCACUCUCUUGUGAGGGACAAAAAUACAAUCAGGGACAGUAUAUGAA |
| | | UACUCCAUGGAGAAACCCAGCUAAAGAGAGAGAAAAAUUAGCAUACAGAA |
| | | AACAAAAUAUGGAUGAUAUAGAUAAGGAAGAUGAUGACUUGGUAGGGGUA |
| | | UCAGUGAGGCCAAAAGUUCCCCUAAGAACAAUGAGUUACAAAUUGGCAAU |
| | | AGACAUGUCUCAUUUUAUAAAAGAAAAGGGGGGACUGGAAGGGAUUUAUU |
| | | ACAGUGCAAGAAGACAUAGAAUCUUAGACAUAUACUUAGAAAAGGAAGAA |
| | | GGCAUCAUACCAGAUUGGCAGGAUUACACCUCAGGACCAGGAAUUAGAUA |
| | | CCCAAAGACAUUUGGCUGGCUAUGGAAAUUAGUCCCUGUAAAUGUAUCAG |
| | | AUGAGGCACAGGAGGAUGAGGAGCAUUAUUUAACGCAUCCAGCUCAAACU |
| | | UCCCAGUGGGAUGACCCUUGGGGAGAGGUUCUAGCAUGGAAGUUUGAUCC |
| | | AACUCUGGCCUACACUUAUGAGGCAUAUGUUAGAUACCCAGAAGAGUUUG |
| | | GAAGCAAGUCAGGCCUGUCAGAGGAAGAGGUUAAAAGAAGGCUAACCGCA |
| | | AGAGGCCUUCUUAACAUGGCUGACAAGAAGGAAACUCGCUGAAUUCGAGC |
| | | UAUCUACAGGGGACUUUCCGCUGGGGACUUUCCAGGGAGGCGUGGCCUGG |
| | | GCGGGACCGGGGAGUGGCGAGCCCUCAGAUGCUGCAUAUAAGCAGCCGCU |
| | | UUUGCCUGUACUGGGUCUCUCUAGUUAGACCAGAUCUGAGCCUGGGAGCU |
| | | CUCUGGCUAGCUGAGAACCCACUGCUUAGGCCUCAAUAAAGCUUGCCUUG |
| | | AGUGCUGUAAGUAGUGUGUGCCCGUCUGUUGUGUGACUCUGGUAACUAGA |
| | | GAUCCCUCAGACCAUUUUAGUCAGUGUGGAAAAUCUCUAGCA |
| SEQ ID | clone 1.26 | UGGAAGGGAUUUAUUACAGUGCAAGAAGACAUAGAAUCUUAGACAUAUAC |
| NO: 55 | RNA | UUAGAAAAGGAAGAAGGCAUCAUACCAGAUUGGCAGGAUUACACCUCAGG |
| | | ACCAGGAAUUAGAUACCCAAAGACAUUUGGCUGGCUAUGGAAAUUAGUCC |
| | | CUGUAAAUGUAUCAGAUGAGGCACAGGAGGAUGAGGAGCAUUAUUUAAUG |
| | | CAUCCAGCUCAAACUUCCCAGUGGGAUGACCCUUGGGGAGAGGUUCUAGC |
| | | AUGGAAGUUUGAUCCAACUCUGGCCUACACUUAUGAGGCAUACGUUAGAU |
| | | ACCCAGAAGAGUUUGGAAGCAAGUCAGGCCUGUCAGAGGAAGAGGUUAAA |
| | | AGAAGGCUAACCGCAAGAGGCCUUCUUAACAUGGCUGACAAGAAGGAAAC |
| | | UCGCUGAAUUCGAGCUAUCUACAGGGGACUUUCCGCUGGGGACUUUCCAG |
| | | GGAGGCGUGGCCUGGGCGGGACCGGGGAGUGGCGAGCCCUCAGAUGCUGC |
| | | AUAUAAGCAGCCGCUUUUGCCUGUACUGGGUCUCUCUAGUUAGACCAGAU |
| | | CUGAGCCUGGGAGCUCUCUGGCUAGCUGAGAACCCACUGCUUAAGCCUCA |
| | | AUAAAGCUUGCCUUGAGUGCUUUAAGUAGUGUGUGCCCGUCUGUUGUGUG |
| | | ACUCUGGUAACUAGAGAUCCCUCAGACCAUUUUAGUCAGUGUGGAAAAUC |
| | | UCUAGCAGUGGCGCCCGAACAGGGACCGGAAAGCGAAAGAGAAACCAGAG |
| | | AAGCUCUCUCGACGCAGGACUCGGCUUGCUGAAGCGCGCACGGCAAGCGG |
| | | CGAGGGGCAGCGACCGGUGAGUACGCUAAAAAUUUUGACUAGCGGAGGCU |
| | | AGAAGGAGAGAGAUGGGUGCGAGAGCGUCAGUAUUAAGCGGGGGAAAAUU |
| | | GGAUGCAUGGGAAAAAAUUCGGUUACGGCCAGGAGGAAAGAAAAAAUAUA |
| | | GACUAAAACAUCUAGUAUGGGCAAGCAGGGAGCUAGAACGAUUUGCACUU |
| | | AAUCCUGGCCUUUUAGAGACAUCAGAUGGCUGUAAACAAAUAAUAGGACA |
| | | GCUACAACCAGCUAUCCGGACAGGAUCAGAAGAACUUAGAUCAUUAUUUA |
| | | AUACAGUAGCAACCCUCUAUUGUGUACAUGAAAGGAUAGAGGUAAAAGAC |
| | | ACCAAGGAAGCUUUAGAGAAGAUAGAGGAAGAGCAAAACAAAAGUAAGAA |
| | | AAAAGCACAGCAAGCAGCAGCUGACACAGGACACAGCAAUCAGGUCAGCC |
| | | AAAAUUACCCUAUAGUGCAGAACAUCCAGGGGCAAAUGGUACAUCAGGCC |
| | | CUAUCACCUAGAACUUUAAAUGCGUGGGUAAAAGUAGUAGAAGAGAAGGC |
| | | UUUUAGCCCAGAAGUAAUACCCAUGUUUUCAGCAUUAUCAGAAGGAGCCA |
| | | CCCCACAAGAUUUAAACACCAUGCUAAACACAGUGGGGGGACAUCAAGCA |
| | | GCCAUGCAAAUGUUAAAAGAGACCAUCAAUGAGGAAGCUGCAGAAUGGGA |
| | | UAGAGUGCAUCCAGUGCAUGCAGGGCCUAUUGCACCAGGCCAGAUGAGAG |
| | | AACCAAGGGGAAGUGACAUAGCAGGAACUACUAGUACCCUUCAGGAACAA |
| | | AUAGGAUGGAUGACACAUAAUCCACCUAUCCCAGUAGGAGAAAUCUAUAA |
| | | AAGAUGGAUAAUCCUGGGAUUAAAUAAAAUAGUAAGAAUGUAUAGCCCUA |
| | | CCAGCAUUCUGGACAUAAGACAAGGACCAAAGGAACCCUUUAGAGACUAU |
| | | GUAGACCGGUUCUAUAAAACCCUAAGAGCCGAGCAAGCUACACAGGAGGU |
| | | AAAAAAUUGGAUGACAGAAACCUUGUUGGUCCAAAAUGCGAACCCAGAUU |
| | | GUAAAACUAUUUUAAAAGCAUUGGGACCAGCAGCCACACUAGAAGAAAUG |
| | | AUGACAGCAUGUCAGGGAGUGGGAGGACCCGGCCAUAAAGCAAGAGUUUU |
| | | GGCUGAAGCAAUGAGCCAAGUAACAAAUUCAGCUACCAUAAUGAUGCAGA |
| | | GAGGCAAUUUUAGGAACCAAAGAAAAACUGUUAAGUGUUUCAAUUGUGGC |
| | | AAAGAAGGGCACAUAGCCAAAAAUUGCAGGGCUCCUAGGAAAAAGGGCUG |
| | | UUGGAAAUGUGGAAAGGAAGGACACCAAAUGAAAGAUUGUACUGAGAGAC |
| | | AGGCUAAUUUUUUAGGGAAGAUCUGGCCUUCCCACAAGGGAAGGCCACGA |
| | | AAUUUUCUUCAGAGCAGACCAGAGCCAACAGCCCCAUCAGAAGAGAGCGU |
| | | CAAGUUUGGAGAAGAGACAACAACUCCCUCUCAGAAGCAGGAGCCGAUAG |
| | | ACAAGGAACUGUAUCCUUUAACUUCCCUCAGAUCACUCUUUGGCAACGAC |
| | | CCCUCGUCACAAUAAAGAUAGGGGGGCAACUAAAGGAAGCUCUAUUAGAU |
| | | ACAGGAGCAGAUGAUACAGUAUUAGAAGACAUGGAUUUGCCAGGAAGAUG |
| | | GAAACCAAAAAUGAUAGGGGGAAUUGGAGGUUUUAUCAAAGUAAGACAGU |
| | | ACGAUCAGAUACCCAUAGAUAUCUGUGGACAUAAAGCUGUAGGUACAGUA |
| | | UUAGUAGGACCUACACCUGUCAACAUAAUUGGAAGAAAUCUGUUGACUCA |
| | | GAUUGGUUGCACUUUAAAUUUUCCCAUUAGUCCUAUUGAAACUGUACCAG |
| | | UAAAAUUAAAGCCAGGAAUGGAUGGCCCAAAAGUCAAACAAUGGCCAUUG |
| | | ACAGAAGAAAAAAUAAAAGCAUUAGUAGAAAUUUGUACAGAAAUGGAAAA |
| | | GGAAGGAAAGAUUUCAAAAAUUGGGCCUGAAAAUCCAUACAAUACUCCAG |
| | | UAUUUGCCAUAAAGAAAAAAGACAGUACUAAAUGGAGAAAAUUAGUAGAU |
| | | UUCAGAGAACUUAAUAGGAAAACUCAAGACUUCUGGGAAGUUCAAUUAGG |
| | | AAUACCACAUCCCGCAGGGUUAAAAAAGAAAAAAUCAGUAACAGUACUGG |
| | | AUGUGGGUGAUGCAUAUUUUUCAGUUCCCUUAGAUAAAGACUUCAGGAAG |
| | | UAUACUGCAUUUACCAUACCUAGUAUAAACAAUGAGACACCAGGGAUUAG |
| | | AUAUCAGUACAAUGUGCUUCCACAGGGAUGGAAAGGAUCACCAGCAAUAU |
| | | UCCAAAGUAGCAUGACAAAAACCUUAGAGCCUUUUAGAAAACAAAAUCCA |
| | | GACAUAAUUAUCUAUCAAUACAUGGAUGAUUUGUAUGUAGGAUCUGACUU |
| | | AGAAAUAGGGCAGCAUAGAACAAAAAUAGAGGAACUGAGACAACAUCUGU |
| | | UAAAGUGGGGAUUUACCACACCAGACAAAAAACAUCAGAAAGAACCUCCA |
| | | UUCCUUUGGAUGGGUUAUGAACUCCAUCCUGAUAAAUGGACAGUACAGCC |
| | | UAUAGUGCUGCCAGAAAAAGACAGCUGGACUGUCAAUGACAUACAGAAGU |
| | | UAGUGGGAAAAUUAAAUUGGGCAAGUCAAAUUUAUGCAGGGAUUAAAGUA |
| | | AAGCAAUUAUGUAAACUCCUUAGGGGAACCAAAGCACUUACAGAAGUAAU |
| | | ACCACUAACAAAAGAAGCAGAGCUAGAACUGGCAGAAAACAGGGAGAUUU |
| | | UAAAGGAACCAGUACAUGGAGUGUAUUAUGACCCAUCAAAAGACUUAAUA |
| | | GUAGAAAUACAGAAGCAGGGGCAAGGCCAAUGGACAUAUCAAAUUUUUCA |
| | | AGAGCCAUUUAAAAAUCUGAAAACAGGAAAAUAUGCAAAAACGAGGGGUG |
| | | CCCACACUAAUGAUGUAAAACAAUUAACAGAGGCAGUGCAAAAAAUAGCC |
| | | AAUGAAAGCAUAGUAAUAUGGGGAAAGAUUCCUAAAUUUAAAUUACCCAU |
| | | ACAAAAAGAAACAUGGGAAACAUGGUGGACAGAGUAUUGGCAAGCCACCU |
| | | GGAUUCCUGAGUGGGAGUUUGUCAAUACCCCUCCCUUAGUGAAAUUAUGG |
| | | UACCAGUUAGAAAAAGAACCCAUAGUAGGAGCAGAAACUUUCUAUGUAGA |
| | | UGGGGCAGCUAACAGGGAGACUAAAUUAGGAAAAGCAGGAUAUGUUACUA |
| | | GCAGAGGAAGGCAAAAAGUUGUCUCCCUAACAGACACAACAAAUCAGAAA |
| | | ACUGAGUUACAAGCAAUUCACCUAGCUUUGCAGGAUUCACGAUUAGAAGU |
| | | AAACAUAGUAACAGACUCACAAUAUGCAUUAGGAAUCAUUCAAGCACAAC |
| | | CAGAUAAAAGUGAAUCAGAGUUAGUCAGUCAAAUAAUAGAACAGCUAAUA |
| | | AAAAAGGAAAAAGUCUACCUGGCAUGGGUACCAGCACACAAAGGAAUUGG |
| | | AGGAAAUGAACAGGUAGAUAAAUUAGUCAGUGCUGGAAUCAGGAGAGUAC |
| | | UAUUUCUAGAUGGAAUAGAGAAGGCCCAAGAAGAACAUGAGAAAUAUCAU |
| | | AAUAAUUGGAGAGCAAUGGCUAGUGAAUUUAACCUGCCAGCUGUAGUAGC |
| | | AAAAGAGAUAGUAGCCUGCUGUGAUAAGUGCCAGGUAAAAGGAGAAGCCA |
| | | UGCAUGGACAAGUAGACUGCAGUCCAGGAAUAUGGCAACUAGAUUGUACA |
| | | CAUUUAGAAGGAAAAGUUAUCCUGGUAGCAGUUCAUGUAGCCAGUGGAUA |
| | | UAUAGAAGCAGAGGUUAUUCCAGCAGAGACAGGACAGGAGACAGCAUACU |
| | | UUAUUUUAAAAUUAGCAGGAAGAUGGCCAGUAAAAACAAUACAUACAGAC |
| | | AAUGGCAGUAAUUUCACCAGUACUACGGUUAAGGCCGCCUGUUGGUGGGC |
| | | AGGGAUCAAGCAGGAAUUUGGCAUUCCCUACAAUCCCCAAAGUCAAGGAG |
| | | UAGUAGAAUCUAUGAAUAAAGAAUUAAAGAAAAUUAUAGAACAAGUAAGA |
| | | GAUCAGGCUGAACAUCUUAAGACAGCAGUACAAAUGGCAGUAUUCAUUCA |
| | | CAAUUUUAAAAGAAAAGGGGGGAUUGGGGGGUACAGUGCAGGGGAAAGAA |
| | | UAGUAGACAUAAUAGCAUCAGACAUACAAACUAAAGAACUACAAAAACAA |
| | | AUUACAAAAAUUCAAAAUUUUCGGGUUUAUUACAGGGACAGCAGAGAUCC |
| | | ACUUUGGAAAGGACCAGCAAAGCUUCUUUGGAAAGGUGAAGGGGCAGUAG |
| | | UAAUACAAGAUAAGAGUGACAUAAAAGUAGUGCCAAGAAGAAAAGCAAAG |
| | | AUUAUCAGGGAUUAUGGAAAACAGAUGGCAGGUGAUGAUUGUGUGGCAAG |
| | | UAGACAGGAUGAGGAUUAGAACAUGGAAAAGUUUAGUAAAACACCAUAUG |
| | | UAUGUUUCAAAGAAAGCUAAGGGAUGGUUUUAUAGACAUCACUAUAAAAG |
| | | CACUCAUCCAAGAAUAAGUUCAGAAGUACAUAUCCCACUAGGGGAUGCUA |
| | | GCUUGGUAGUAACAACAUAUUGGGGUCUACAUACAGGAGAAAGAGACUGG |
| | | CAUUUGGGUCAGGGAGUCUCCAUAGAAUGGAGGAAAAGGAGAUACAGCAC |
| | | ACAAGUAGACCCUGACCUAGCAGACCAACUAAUUCAUCUGUACUACUUUG |
| | | AUUGUUUUUCAGAAUCUGCUAUAAGAAAUGCCAUAUUAGGACAUAGAGUU |
| | | AGUCCUAGGUGUGAAUAUCAAGCAGGACAUAACAAGGUAGGAUCUCUACA |
| | | GUACUUGGCACUAGCAGCAUUAGUAACACCAAGAAAGAUAAAGCCACCUU |
| | | UGCCUAGUGUUGCGAAACUGACAGAGGACAGAUGGAACAAGUCCCACAAG |
| | | ACCAAGGGCCACAGAGGGAGCCAUACAAUGAAUGGACACUAAAGCUUUUA |
| | | GAGGAGCUUAAGAAUGAAGCUGUCAGACAUUUCCCUAGACCAUGGCUUCA |
| | | UGGCCUAGGGCAAUAUAUCUAUGAAACUUAUGAGGAUACUUGGGCAGGAG |
| | | UGGAAGCCAUAAUAAGAAUUCUGCAACAAUUGCUGCUUAUUCAUUUCAGA |
| | | AUUGGGUGUCAACAUAGCAGAAUAGGCAUUAUUCGACAGAGGAGAACAAG |
| | | AAAUGGAGCCAGUAGAUCCUAGACUAGAGCCCUGGAAGCAUCCAGGAAGU |
| | | CAGCCUAAGACUGCCUGUACCAAUUGCUAUUGCAAAAAGUGUUGCUUGCA |
| | | UUGCCAAGUUUGCUUCAUAACAAAAGGCUUAGGCAUCUCCUAUGGCAGGA |
| | | AGAAGCGGAAAAAGCGACGAAGAUCUCCUCAACACAGUCAGACUGAUCAA |
| | | GCUUCUCUAUCAAAGCAGUAAGUAGUACAUGUAAUGCAACCUUUAGUAAU |
| | | AUUAGCAAUAGUAGCAUUAGUAGUAGCACUAAUAAUAGUCAUAGUUGUAU |
| | | GGUCCAUUGUAUUAAUAGAAUAUAGAAAAAUAUUAAGACAAAAGAAAAUA |
| | | GACAGGUUAAUUGAUAGAAUAAGAGAAAAAGCAGAAGACAGUGGCAAUGA |
| | | GAGUGAUGGGGAUCAGGAAGAAUUAUCAGCACUUGUGGAAAGGGGGCACC |
| | | UUGCUCCUUGGGAUAUUGAUGAUCUGUAGUGCUGCAGAACAAUUGUGGGU |
| | | CACAGUCUAUUAUGGGGUACCUGUGUGGAAAGAAGCAAACACCACUCUAU |
| | | UUUGUGCAUCAGAUGCUAAGGCAUAUGAUACAGAGGUACAUAAUGUUUGG |
| | | GCCACACAUGCCUGUGUACCCACAGACCCCAACCCACAAGAAAUACUAUU |
| | | GGAAAAUGUGACAGAAGAUUUUAACAUGUGGAAAAAUAACAUGGUAGAAC |
| | | AGAUGCAUGAGGAUAUAAUCAGUUUAUGGGAUCAAAGUCUAAAGCCAUGU |
| | | GUAAAAUUAACCCCACUCUGUGUUACUUUACAUUGCACUGAUUUGAAGAA |
| | | UGGUACUAAUUUGAAGAAUGGUACUAAAAUCAUUGGGAAAUCAAUAAGAG |
| | | GAGAAAUAAAAAACUGCUCUUUCAAUGUCACCAAAAACAUAAUAGAUAAG |
| | | GUGAAAAAAGAAUAUGCGCUUUUCUAUAGACAUGAUGUAGUACCAAUAGA |
| | | UAGGAAUAUUACUAGCUAUAGGUUAAUAAGUUGUAACACCUCAACCCUUA |
| | | CACAGGCCUGUCCAAAGGUAUCCUUUGAGCCAAUUCCCAUACAUUAUUGU |
| | | GCCCCGGCUGGUUUUGCGAUUCUAAAAUGUAAAGAUAAGAAGUUCAAUGG |
| | | AACGGGACCGUGUACAAAUGUCAGUACAGUACAAUGUACACAUGGAAUUA |
| | | GGCCAGUAGUAUCAACUCAACUGCUGUUAAAUGGAAGUCUAGCAGAAGGA |
| | | GAGGUAGUAAUUAGAUCUAGCAAUUUCACGGACAAUGCUAAAAUCAUAAU |
| | | AGUACAGCUGAAUGAAGCUGUAGAAAUUAAUUGUACAAGACCCAACAACA |
| | | AUACAAGAAAAGGGAUAACUCUAGGACCAGGGAGAGUAUUUUAUACAACA |
| | | GGAAAAAUAGUAGGAGAUAUAAGAAAAGCACAUUGUAACAUUAGUAAAGU |
| | | AAAAUGGCAUAACACUUUAAAAAGGGUAGUUAAAAAAUUAAGAGAAAAAU |
| | | UUGAAAAUAAAACAAUAAUCUUUAAUAAAUCCUCAGGGGGGGACCCAGAA |
| | | AUUGUAAUGCACAGCUUUAAUUGUGGAGGGGAAUUUUUCUACUGUAAUAC |
| | | AAAAAAACUGUUUAAUAGUACUUGGAAUGGUACUGAAGGGUCAUAUAACA |
| | | UUGAAGGAAAUGACACUAUCACACUCCCAUGCAGAAUAAAACAAAUUAUA |
| | | AACAUGUGGCAGGAAGUAGGAAAAGCAAUGUAUGCCCCUCCCAUCAGUGG |
| | | ACAAAUUUGGUGCUCAUCAAAUAUUACAGGGCUGCUACUAACAAGAGAUG |
| | | GUGGUAAGAACAGCAGCACCGAAAUCUUCAGACCUGGAGGAGGAGAUAUA |
| | | AGGGACAAUUGGAGAAGUGAAUUAUAUAAAUAUAAAGUAGUAAGAGUUGA |
| | | ACCAUUAGGAAUAGCACCCACCAAGGCAAAAAGAAGAGUGGUGCAGAGAG |
| | | AAAAAAGAGCAGUGGGAAUAGGAGCUGUGUUCCUUGGGUUCUUGGGAGCA |
| | | GCAGGAAGCACUAUGGGCGCAGCGUCAAUAACGCUGACGGUACAGGCCAG |
| | | ACAAUUAUUGUCUGGUAUAGUGCAACAGCAGAACAAUUUGCUGAGGGCUA |
| | | UUGAAGCGCAACAGCAUAUGUUGCAACUCACAGUCUGGGGCAUCAAGCAG |
| | | CUCCAGGCAAGAGUCCUGGCUGUGGAAAGAUACCUACAGGAUCAACAGCU |
| | | CCUGGGGAUUUGGGGUUGCUCUGGAAAACUCAUCUGCACCACUACUGUGC |
| | | CUUGGAAUACUAGUUGGAGUAAUAAAUCUCUGGAUACAAUUUGGGGUAAC |
| | | AUGACCUGGAUGCAGUGGGAAAAAGAAAUUAACAAUUACACAGGCUUAAU |
| | | AUACAACUUGAUUGAAGAAUCGCAGAACCAACAAGAAAAGAAUGAACAAG |
| | | AAUUAUUGGCAUUAGAUAAAUGGGCAAGUUUGUGGAACUGGUUUAACAUA |
| | | UCAAACUGGCUGUGGUAUAUAAAAAUAUUCAUAAUGAUAGUAGGACGCUU |
| | | GAUAGGUUUAAGAAUAGUUUUCAGUGUACUUUCUAUAGUGAAUAGAGUUA |
| | | GGCAGGGAUACUCACCAUUAUCGUUUCAGACCCGCUUCCCAGCCUCGAGG |
| | | GGACCCGACAGGCCCGAAGGAAUCGAAGAAGAAGGUGGAGACAGAGACAG |
| | | AGACAGAUCCAGUCCAUUAGUGGAUGGAUUCUUAGCAAUCAUCUGGGUCG |
| | | ACCUGCGGAGCCUGUUCCUCUUCAGCUACCACCGCUUGAGAGACUUACUC |
| | | UUGAUUGUAACGAGGAUUGUGGAACUUCUGGGACGCAGGGGGUGGGAACU |
| | | CCUCAAAUACUUGUGGAAUCUCCUGCAGUAUUGGAGUCAGGAACUAAAGA |
| | | AUAGUGCUGUUAGCUUGCUUAACGCCACAGCCAUAGCAGUAGGUGAGGGA |
| | | ACAGAUAGAAUUAUAGAAAUAUUACAAAGAGCUGGUAGAGCUAUUCUCAA |
| | | CAUACCUACGAGAAUAAGACAGGGCUUAGAAAGGGCUUUGCUAUAAGCUU |
| | | AUGGGUGGAGCUAUUUCCAUGAGGCGGUCCAGGCCGUCUGGAAAUCUGUA |
| | | CGAGAGACUCUUGCGGGCGCGUGGGGAGACUUAUGGAAAACUCUUAGGAG |
| | | AGGUAAAAGAUGGAUACUCGCAAUCCCCAGGAGGAUUAGACAAGGGCUUG |
| | | AGCUCACUCUCUUGUGAGGGACAAAAAUACAAUCAGGGACAGUAUAUGAA |
| | | UACUCCAUGGAGAAACCCAGCUAAAGAGAGAGAAAAAUUAGCAUACAGAA |
| | | AACAAAAUAUGGAUGAUAUAAAUAAGGAAGAUGAUGACUUGGUAGGGGUA |
| | | UCAGUGAGGCCAAAAGUUCCCCUAAGAACAAUGAGUUACAAAUUGGCAAU |
| | | AGACAUGUCUCAUUUUAUAAAAGAAAAGGGGGGACUGGAAGGGAUUUAUU |
| | | ACAGUGCAAGAAGACAUAGAAUCUUAGACAUAUACUUAGAAAAGGAAGAA |
| | | GGCAUCAUACCAGAUUGGCACGAUUACACCUCAGGACCAGGAAUUAGAUA |
| | | CCCAAAGACAUUUGGCUGGCUAUGGAAAUUAGUCCCUGUAAAUGUAUCAG |
| | | AUGAGGCACAGGAGGAUGAGGAGCAUUAUUUAAUGCAUCCAGCUCAAACU |
| | | UCCCAGUGGGAUGACCCUUGGGGAGAGGUUCUAGCAUCGAAGUUUGAUCC |
| | | AACUCUGGCCUACACUUAUGAGGCAUAUGUUAGAUACCCAGAAGAGUUUG |
| | | GAAGCAAGUCAGGCCUGUCAGAGGAAGAGGUUAAAAGAAGGCUAACCGCA |
| | | AGAGGCCUUCUUAACAUGGCUGACAAGAAGGAAACUCGCUGAAUUCGAGC |
| | | UAUCUACAGGGGACUUUCCGCUGGGGACUUUCCAGGGAGGCGUGGCCUGG |
| | | GCGGGACCGGGGAGUGGCGAGCCCUCAGAUGCUGCAUAUAAGCAGCCGCU |
| | | UUUGCCUGUACUGGGUCUCUCUAGUUAGACCAGAUCUGAGCCUGGGAGCU |
| | | CUCUGGCUAGCUGAGAACCCACUGCUUAAGCCUCAAUAAAGCUUGCCUUG |
| | | AGUGCUUUAAGUAGUGUGUGCCCGUCUGUUGUGUGACUCUGGUAACUAGA |
| | | GAUCCCUCAGACCAUUUUAGUCAGUGUGGAAAAUCUCUAGCA |
| SEQ ID | clone | UGGAAGGGAUUUAUUACAGUGCAAGAAGACAUAGAAUCUUAGACAUAUAC |
| NO: 56 | P8A26 RNA | UUAGAAAAGGAAGAAGGCAUCAUACCAGAUUGGCAGGAUUACACCUCAGG |
| | | ACCAGGAAUUAGAUACCCAAAGACAUUUGGCUGGCUAUGGAAAUUAGUCC |
| | | CUGUAAAUGUAUCAGAUGAGGCACAGGAGGAUGAGGAGCAUUAUUUAAUG |
| | | CAUCCAGCUCAAACUUCCCAGUGGGAUGACCCUUGGGGAGAGGUUCUAGC |
| | | AUGGAAGUUUGAUCCAACUCUGGCCUACACUUAUGAGGCAUAUGUUAAAU |
| | | ACCCAGAAGAGUUUGGAAGCAAGUCAGGCCUGUCAGAGGAAGAGGUUAGA |
| | | AGAAGGCUAACCGCAAGAGGCCUUCUUAACAUGGCUGACAAGAAGGAAAC |
| | | UCGCUGAAUUCGAGCUAUCUACAGGGGACUUUCCGCUGGGGACUUUCCAG |
| | | GGAGGCGUGGCCUGGGCGGGACCGGGGAGUGGCGAGCCCUCAGAUGCUGC |
| | | AUAUAAGCAGCCGCUUUUGCCUGUACUGGGUCUCUCUAGUUAGACCAGAU |
| | | CUGAGCCUGGGAGCUCUCUGGCUAGCUGAGAACCCACUGCUUAAGCCUCA |
| | | AUAAAGCUUGCCUUGAGUGCUUUAAGUAGUGUGUGCCCGUCUGUUGUGUG |
| | | ACUCUGGUAACUAGAGAUCCCUCAGACCAUUUUAGUCAGUGUGGAAAAUC |
| | | UCUAGCAGUGGCGCCCGAACAGGGACCGGAAAGCGAAAGAGAAACCAGAG |
| | | AAGCUCUCUCGACGCAGGACUCGGCUUGCUGAAGCGCGCACGGCAAGCGG |
| | | CGAGGGGCAGCGACCGGUGAGUACGCUAAAAAUUUUGACUAGCGGAGGCU |
| | | AGAAGGAGAGAGAUGGGUGCGAGAGCGUCAGUAUUAAGCGGGGGAAAAUU |
| | | GGAUGCAUGGGAAAAAAUUCGGUUACGGCCAGGAGGAAAGAAAAAAUAUA |
| | | GACUAAAACAUCUAGUAUGGGCAAGCAGGGAGCUAGAACGAUUUGCACUU |
| | | AAUCCUGGCCUUUUAGAGACAUCAGAUGGCUGUAAACAAAUAAUAGGACA |
| | | GCUACAACCAGCUAUCCGGACAGGAUCAGAAGAACUUAGAUCAUUAUUUA |
| | | AUACAGUAGCAACCCUCUAUUGUGUACAUGAAAGGAUAGAGGUAAAAGAC |
| | | ACCAAGGAAGCUUUAGAGAAGAUAGAGGAAGAGCAAAACAAAAGUAAGAA |
| | | AAAAGCACAGCAAGCAGCAGCUGACACAGGACACAGCAAUCAGGUCAGCC |
| | | AAAAUUACCCUAUAGUGCAGAACAUCCAGGGGCAAAUGGUACAUCAGGCC |
| | | CUAUCACCUAGAACUUUAAAUGCGUGGGUAAAAGUAGUAGAAGAGAAGGC |
| | | UUUUAGCCCAGAAGUAAUACCCAUGUUUUCAGCAUUAUCAGAAGGAGCCA |
| | | CCCCACAAGAUUUAAACACCAUGCUAAACACAGUGGGGGGACAUCAAGCA |
| | | GCCAUGCAAAUGUUAAAAGAGACCAUCAAUGAGGAAGCUGCAGAAUGGGA |
| | | UAGAGUGCAUCCAGUGCAUGCAGGGCCUAUUGCACCAGGCCAGAUGAGAG |
| | | AACCAAGGGGAAGUGACAUAGCAGGAACUACUAGUACCCUUCAGGAACAA |
| | | AUAGGAUGGAUGACACAUAAUCCACCUAUCCCAGUAGGAGAAAUCUAUAA |
| | | AAGAUGGAUAAUCCUGGGAUUAAAUAAAAUAGUAAGAAUGUAUAGCCCUA |
| | | CCAGCAUUCUGGACAUAAGACAAGGACCAAAGGAACCCUUUAGAGACUAU |
| | | GUAGACCGGUUCUAUAAAACCCUAAGAGCCGAGCAAGCUACACAGGAGGU |
| | | AAAAAAUUGGAUGACAGAAACCUUGUUGGUCCAAAAUGCGAACCCAGAUU |
| | | GUAAAACUAUUUUAAAAGCACUGGGACCAGCAGCUACACUAGAAGAAAUG |
| | | AUGACAGCAUGUCAGGGAGUGGGAGGACCCGGCCAUAAAGCAAGAGUUUU |
| | | GGCUGAAGCAAUGAGCCAAGUAACAAAUUCAGCUACCAUAAUGAUGCAGA |
| | | GAGGCAAAUUUAGGAACCAAAGAAAAACUGUUAAGUGUUUCAAUUGUGGC |
| | | AAAGAAGGGCACAUAGCCAAAAAUUGCAGGGCUCCUAGGAAAAAGGGCUG |
| | | UUGGAAAUGUGGAAAGGAAGGACACCAAAUGAAAGAUUGUACUGAGAGAC |
| | | AGGCUAAUUUUUUAGGGAAGAUCUGGCCUUCCCACAAGGGAAGGCCAGGA |
| | | AAUUUUCUUCAGAGCAGACCAGAGCCAACAGCCCCAUCAGAAGAGAGCGU |
| | | CAGGUUUGGAGAGGAGACAACAACUCCCUCUCAGAAGCAGGAGCCGAUAG |
| | | ACAAGGAACUGUAUCCUUUAACUUCCCUCAGAUCACUCUUUGGCAACGAC |
| | | CCCUCGUCACAAUAAAGAUAGGGGGGCAACUAAAGGAAGCUCUAUUGGAU |
| | | ACAGGAGCAGAUGAUACAGUAUUAGAAGACAUGGAUUUGCCAGGAAGAUG |
| | | GAAACCAAAAAUGAUAGGGGGAAUUGGAGGUUUUAUCAAAGUAAGACAGU |
| | | AUGAUCAGAUACCCAUAGAUAUCUGUGGACAUAAAGCUGUAGGUACAGUA |
| | | UUAGUAGGACCUACACCUGUCAACAUAAUUGGAAGAAAUCUGUUGACUCA |
| | | GAUUGGUUGCACUUUAAAUUUUCCCAUUAGUCCUAUUGAAACUGUACCAG |
| | | UAAAAUUAAAGCCAGGAAUGGAUGGCCCAAAAGUCAAACAAUGGCCAUUG |
| | | ACAGAAGAAAAAAUAAAAGCAUUAGUAGAAAUUUGUACAGAAAUGGAAAA |
| | | GGAAGGAAAGAUUUCAAAAAUUGGGCCUGAAAAUCCAUACAAUACUCCAG |
| | | UAUUUGCCAUAAAGAAAAAAGACAGUACUAAAUGGAGAAAAUUAGUAGAU |
| | | UUCAGAGAACUUAAUAGGAAAACUCAAGACUUCUGGGAAGUUCAAUUAGG |
| | | AAUACCACAUCCCGCAGGGUUAAAAAAGAAAAAAUCAGUAACAGUACUGG |
| | | AUGUGGGUGAUGCAUAUUUUUCAGUUCCCUUAGAUAAAGACUUCAGGAAG |
| | | UAUACUGCAUUUACCAUACCUAGUAUAAACAAUGAGACACCAGGGAUUAG |
| | | AUAUCAGUACAAUGUGCUUCCACAGGGAUGGAAAGGAUCACCAGCAAUAU |
| | | UCCAAAGUAGCAUGACAAAAACCUUAGAGCCUUUUAGAAAACAAAAUCCA |
| | | GACAUAAUUAUCUAUCAAUACAUGGAUGAUUUGUAUGUAGGAUCUGACUU |
| | | AGAAAUAGGGCAGCAUAGAACAAAAAUAGAGGAACUGAGACAACAUCUGU |
| | | UGAAGUGGGGAUUUACCACACCAGACAAAAAACAUCAGAAAGAACCUCCA |
| | | UUCCUUUGGAUGGGUUAUGAACUCCAUCCUGAUAAAUGGACAGUACAGCC |
| | | UAUAGUGCUGCCAGAAAAAGACAGCUGGACUGUCAAUGACAUACAGAAGU |
| | | UAGUGGGAAAAUUAAAUUGGGCAAGUCAAAUUUAUGCAGGGAUUAAAGUA |
| | | AAGCAAUUAUGUAAACUCCUUAGGGGAACCAAAGCACUUACAGAAGUAAU |
| | | ACCACUAACAAAAGAAGCAGAGCUAGAACUGGCAGAAAACAGGGAGAUUC |
| | | UAAAGGAACCAGUACAUGGAGUGUAUUAUGACCCAUCAAAAGACUUAAUA |
| | | GUAGAAAUACAGAAGCAGGGGCAAGGCCAAUGGACAUAUCAAAUUUUUCA |
| | | AGAGCCAUUUAAAAAUCUGAAAACAGGAAAAUAUGCAAAAACGAGGGGUG |
| | | CCCACACUAAUGAUGUAAAACAAUUAACAGAGGCAGUGCAAAAAAUAGCC |
| | | AAUGAAAGCAUAGUAAUAUGGGGAAAGAUUCCUAAAUUUAAAUUACCCAU |
| | | ACAAAAAGAAACAUGGGAAACAUGGUGGACAGAGUAUUGGCAAGCCACCU |
| | | GGAUUCCUGAGUGGGAGUUUGUCAAUACCCCUCCCUUAGUGAAAUUAUGG |
| | | UACCAGUUAGAAAAAGAACCCAUAGUAGGAGCAGAAACUUUCUAUGUAGA |
| | | UGGGGCAGCUAACAGGGAGACUAAAUUAGGAAAAGCAGGAUAUGUUACUA |
| | | GCAGAGGAAGGCAAAAAGUUGUCUCCCUAACAGACACAACAAAUCAGAAA |
| | | ACUGAGUUACAAGCAAUUCACCUAGCUUUGCAGGAUUCAGGAUUAGAAGU |
| | | AAACAUAGUAACAGACUCACAAUAUGCAUUAGGAAUCAUUCAAGCACAAC |
| | | CAGAUAAAAGUGAAUCAGAGUUAGUCAGUCAAAUAAUAGAACAGCUAAUA |
| | | AAAAAGGAAAAAGUCUACCUGACAUGGAUACCAGCACACAAAGGAAUUGG |
| | | AGGAAAUGAACAGGUAGAUAAAUUAGUCAGUGCUGGAAUCAGGAGAGUAC |
| | | UAUUUCUAGAUGGAAUAGAGAAGGCCCAAGAAGAACAUGAGAAAUAUCAU |
| | | AGUAAUUGGAGAGCAAUGGCUAGUGAAUUUAACCUGCCAGCUGUAGUAGC |
| | | AAAAGAAAUAGUAGCCUGCUGUGAUAAGUGCCAGGUAAAAGGAGAAGCCA |
| | | UGCAUGGACAAGUAGACUGCAGUCCAGGAAUAUGGCAACUAGAUUGUACA |
| | | CAUUUAGAAGGAAAAGUUAUCCUGGUAGCAGUUCAUGUAGCCAGUGGAUA |
| | | UAUAGAAGCAGAGGUUAUUCCAGCAGAGACAGGACAGGAAACAGCAUACU |
| | | UUAUUUUAAAAUUAGCAGGAAGAUGGCCAGUAAAAACAAUACAUACAGAC |
| | | AAUGGCAGUAAUUUCACCAGUACUACGGUUAAGGCCGCCUGUUGGUGGGC |
| | | AGGGAUCAAGCAGGAAUUUGGCAUUCCCUACAAUCCCCAAAGUCAAGGAG |
| | | UAGUAGAAUCUAUGAAUAAAGAAUUAAAGAAAAUUAUAGAACAAGUAAGA |
| | | GAUCAGGCUGAACAUCUUAAGACAGCAGUACAAAUGGCAGUAUUCAUUCA |
| | | CAAUUUUAAAAGAAAAGGGGGGAUUGGGGGGUACAGUGCAGGGGAAAGAA |
| | | UAGUAGACAUAAUAGCAUCAGACAUACAAACUAAAGAACUACAAAAACAA |
| | | AUUACAAAAAUUCAAAAUUUUCGGGUUUAUUACAGGGACAGCAGAGAUCC |
| | | ACUUUGGAAAGGACCAGCAAAGCUUCUUUGGAAAGGUGAAGGGGCAGUAG |
| | | UAAUACAAGAUAAGAGUGACAUAAAAGUAGUGCCAAGAAGAAAAGCAAAG |
| | | AUUAUCAGGGAUUAUGGAAAACAGAUGGCAGGUGAUGAUUGUGUGGCAAG |
| | | UAGACAGGAUGAGGAUUAGAACAUGGAAAAGUUUAGUAAAACACCAUAUG |
| | | UAUGUUUCAAAGAAAGCUAAGGGAUGGUUUUAUAGACAUCACUAUGAAAG |
| | | CACUCAUCCAAGAAUAAGUUCAGAAGUACAUAUCCCACUAGGGGAUGCUA |
| | | GCUUGGUAGUAACAACAUAUUGGGGUCUACAUACAGGAGAAAGAGACUGG |
| | | CAUUUGGGUCAGGGAGUCUCCAUAGAAUGGAGGAAAAGGAGAUACAGCAC |
| | | ACAAGUAGACCCUGACCUAGCAGACCAACUAAUUCAUCUGUACUACUUUG |
| | | AUUGUUUUUCAGAAUCUGCUAUAAGAAAUGCCAUAUUAGGACAUAGAGUU |
| | | AGUCCUAGGUGUGAAUAUCAAGCAGGACAUAACAAGGUAGGAUCUCUACA |
| | | GUACUUGGCACUAGCAGCAUUAGUAACACCAAGAAAGAUAAAGCCACCUU |
| | | UGCCUAGUGUUGCGAAACUGACAGAGGACAGAUGGAACAAGUCCCACAAG |
| | | ACCAAGGGCCACAGAGGGAGCCAUACAAUGAAUGGACACUAGAGCUUUUA |
| | | GAGGAGCUUAAGAAUGAAGCUGUCAGACAUUUCCCUAGACCAUGGCUUCA |
| | | UGGCCUAGGACAAUAUAUCUAUGAAACUUAUGAGGAUACUUGGGCAGGAG |
| | | UGGAAGCCAUAAUAAGAAUUCUGCAACAAUUGCUGCUUAUUCAUUUCAGA |
| | | AUUGGGUGUCAACAUAGCAGAAUAGGCAUUAUUCGACAGAGGAGAACAAG |
| | | AAAUGGAGCCAGUAGAUCCUAGACUAGAGCCCUGGAAGCAUCCAGGAAGU |
| | | CAGCCUAAGACUGCCUGUACCAAUUGCUAUUGCAAAAAGUGUUGCUUGCA |
| | | UUGCCAAGUUUGCUUCAUAACAAAAGGCUUAGGCAUCUCCUAUGGCAGGA |
| | | AGAAGCGGAAAAAGCGACGAAGAUCUCCUCAACACAGUCAGACUGAUCAA |
| | | GCUUCUCUAUCAAAGCAGUAAGUAGUACAUGUAAUGCAACCUUUGGUAAU |
| | | AUUAGCAAUAGUAGCAUUAGUAGUAGCACUAAUAAUAGUCAUAGUUGUAU |
| | | GGUCCAUUGUAUUAAUAGAAUAUAGAAAAAUAUUAAGACAAAAGAAAAUA |
| | | GACAGGUUAAUUGAUAGAAUAAGAGAAAGAGCAGAAGACAGUGGCAAUGA |
| | | GAGUGAUGGGGAUCAGGAAGAAUUAUCAGCACUUGUGGAAAGGGGGCACC |
| | | UUGCUCCUUGGAAUAUUGAUGAUCUGUAGUGCUGCAGAACAAUUGUGGGU |
| | | CACAGUCUAUUAUGGGGUACCUGUGUGGAAAGAAGCAAACACCACUCUAU |
| | | UUUGUGCAUCAGAUGCUAAAGCAUAUGAUACAGAGGUACAUAAUGUUUGG |
| | | GCCACACAUGCCUGUGUACCCACAGACCCCAACCCACAAGAAAUACUAUU |
| | | GGAAAAUGUGACAGAAGAUUUUAACAUGUGGAAAAAUAACAUGGUAGAAC |
| | | AGAUGCAUGAGGAUAUAAUCAGUUUAUGGGAUCAAAGUCUAAAGCCAUGU |
| | | GUAAAAUUAACCCCACUCUGUGUUACUUUACAUUGCACUGAUUUGAAGAA |
| | | UGGUACUAAUUUGAAGAAUGGUACUAAAAUCAUUGGGAAAUCAAUGAGAG |
| | | GAGAAAUAAAAAACUGCUCUUUCAAUGUCACCAAAAACAUAAUAGAUAAG |
| | | GUGAAAAAAGAAUAUGCGCUUUUCUAUAGACAUGAUGUAGUACCAAUAGA |
| | | UAGGAAUAUUACUAGCUAUAGGUUGAUAAGUUGUAACACCUCAACCCUUA |
| | | CACAGGCCUGUCCAAAGGUAUCCUUUGAGCCAAUUCCCAUACAUUAUUGU |
| | | GCCCCGGCUGGUUUUGCGAUUCUAAAAUGUAAAGAUAAGAAGUUCAAUGG |
| | | AACGGGACCAUGUACAAAUGUCAGUACAGUACAAUGUACACAUGGAAUUA |
| | | GGCCAGUAGUAUCAACUCAACUGCUGUUAAAUGGAAGUCUAGCAGAAGAA |
| | | GAGGUAGUAAUUAGAUCUAGCAAUUUCACGGACAAUGCUAAAAUCAUAAU |
| | | AGUACAGCUGAAUGAAACUGUAGAAAUUAAUUGUACAAGACCCAACAACA |
| | | AUACAAGAAAAGGGAUAACUCUAGGACCAGGGAGAGUAUUUUAUACAACA |
| | | GGAAAAAUAGUAGGAGAUAUAAGAAAAGCACAUUGUAACAUUAGUAAAGU |
| | | AAAAUGGCAUAACACUUUAAAAAGGGUAGUUGAAAAAUUAAGAGAAAAAU |
| | | UUGAAAAUAAAACAAUAAUCUUUAAUAAAUCCUCAGGGGGGGACCCAGAA |
| | | AUUGUAAUGCACAGCUUUAAUUGUGGAGGGGAAUUUUUCUACUGUAAUAC |
| | | AAAAAAACUGUUUAAUAGUACUUGGAAUGGUACUGAAGGGUCAUAUAACA |
| | | UUGAAGGAAAUGACACUAUCACACUCCCAUGCAGAAUAAAACAAAUUAUA |
| | | AACAUGUGGCAGGAAGUAGGAAAAGCAAUGUAUGCCCCUCCCAUCAGUGG |
| | | ACAAAUUUGGUGCUCAUCAAAUAUUACAGGGCUGCUACUAACAAGAGAUG |
| | | GUGGUAAGAACAGCAGCACCGAAAUCUUCAGACCUGGAGGAGGAGAUAUA |
| | | AGGGACAAUUGGAGAAGUGAAUUAUAUAAAUAUAAAGUAGUAAGAGUUGA |
| | | ACCAUUAGGAAUAGCACCCACCAAGGCAAAGAGAAGAGUGGUGCAGAGAG |
| | | AAAAAAGAGCAGUGGGAAUAGGAGCUGUGUUCCUUGGGUUCUUGGGAGCA |
| | | GCAGGAAGCACUAUGGGCGCAGCGUCAAUAACGCUGACGGUACAGGCCAG |
| | | ACAAUUAUUGUCUGGUAUAGUGCAACAGCAGAACAAUUUGCUGAGGGCUA |
| | | UUGAAGCGCAACAGCAUAUGUUGCAACUCACAGUCUGGGGCAUCAAGCAG |
| | | CUCCAGGCAAGAGUCCUGGCUGUGGAAAGAUACCUACAGGAUCAACAGCU |
| | | CCUGGGGAUUUGGGGUUGCUCUGGAAAACUCAUCUGCACCACUACUGUGC |
| | | CUUGGAAUACUAGUUGGAGUAAUAAAUCUCUGGAUACAAUUUGGGGUAAC |
| | | AUGACCUGGAUGCAGUGGGAAAAAGAAAUUAACAAUUACACAGGCUUAAU |
| | | AUACAACUUGAUUGAAGAAUCGCAGAACCAACAAGAAAAGAAUGAACAAG |
| | | AAUUAUUGGCAUUAGAUAAAUGGGCAAGUUUGUGGAAUUGGUUUAACAUA |
| | | UCAAACUGGCUGUGGUAUAUAAAAAUAUUCAUAAUGAUAGUAGGAGGCUU |
| | | GAUAGGUUUAAGAAUAGUUUUCAGUGUACUUUCUAUAGUGAAUAGAGUUA |
| | | GGCAGGGAUACUCACCAUUAUCGUUUCAGACCCGCUUCCCAGCCUCGAGG |
| | | GGACCCGACAGGCCCGAAGGAAUCGAAGAAGAAGGUGGAGACAGAGACAG |
| | | AGACAGAUCCAGUCCAUUAGUGGAUGGAUUCUUAGCAAUCAUCUGGGUCG |
| | | ACCUGCGGAGCCUGUUCCUCUUCAGCUACCACCGCUUGAGAGACUUACUC |
| | | UUGAUUGUAACGAGGAUUGUGGAACUUCUGGGACGCAGGGGGUGGGAACU |
| | | CCUCAAAUACUUGUGGAAUCUCCUGCAGUAUUGGAGUCAGGAACUAAAGA |
| | | AUAGUACUGUUAGCUUGCUUAACGCCACAGCCAUAGCAGUAGGUGAGGGA |
| | | ACAGAUAGGAUUAUAGAAAUAUUACAAAGAGCUGGUAGAGCUAUUCUCAA |
| | | CAUACCUACGAGAAUAAGACAGGGCUUAGAAAGGGCUUUGCUAUAAGCUU |
| | | AUGGGUGGAGCUAUUUCCAUGAGGCGGUCCAGGCCGUCUGGAGAUCUGUA |
| | | CGAGAGACUCUUGCGGGCGCGUGGGGAGACUUAUGGGAGACUCUUAGGAG |
| | | AGGUGGAAGAUGGAUACUCGCAAUCCCCAGGAGGAUUAGACAAAGGCUUG |
| | | AGCUCACUCUCUUGUGAGGGACAGAAAUACAAUCAGGGACAGUAUAUGAA |
| | | UACUCCAUGGAGAAACCCAGCUAAAGAGAAAGAAAAAUUAGCAUACAGAA |
| | | AACAAAAUAUGAAUGAUAUAAAUAAGGAAGAUGAUAACUUGGUAGGGGUA |
| | | UCAGUGAGGCCAAAAGUUCCCCUAAGAACAAUGAGUUACAAAUUGGCAAU |
| | | AGACAUGUCUCAUUUUAUAAAAGAAAAGGGGGGACUGGAAGGGAUUUAUU |
| | | ACAGUGCAAGAAGACAUAGAAUCUUAGACAUAUACUUAGAAAAGGAAGAA |
| | | GGCAUCAUACCAGAUUGGCAGGAUUACACCUCAGGACCAGGAAUUAGAUA |
| | | CCCAAAGACAUUUGGCUGGCUAUGGAAAUUAGUCCCUGUAAAUGUAUCAG |
| | | AUGAGGCACAGGAGGAUGAGGAGCAUUAUUUAAUGCAUCCAGCUCAAACU |
| | | UCCCAGUGGGAUGACCCUUGGGGAGAGGUUCUAGCAUGGAAGUUUGAUCC |
| | | AACUCUGGCCUACACUUAUGAGGCAUAUGUUAAAUACCCAGAAGAGUUUG |
| | | GAAGCAAGUCAGGCCUGUCAGAGGAAGAGGUUAGAAGAAGGCUAACCGCA |
| | | AGAGGCCUUCUUAACAUGGCUGACAAGAAGGAAACUCGCUGAAUUCGAGC |
| | | UAUCUACAGGGGACUUUCCGCUGGGGACUUUCCAGGGAGGCGUGGCCUGG |
| | | GCGGGACCGGGGAGUGGCGAGCCCUCAGAUGCUGCAUAUAAGCAGCCGCU |
| | | UUUGCCUGUACUGGGUCUCUCUAGUUAGACCAGAUCUGAGCCUGGGAGCU |
| | | CUCUGGCUAGCUGAGAACCCACUGCUUAAGCCUCAAAUAAAGCUUGCCUUG |
| | | AGUGCUUUAAGUAGUGUGUGCCCGUCUGUUGUGUGACUCUGGUAACUAGA |
| | | GAUCCCUCAGACCAUUUUAGUCAGUGUGGAAAAUCUCUAGCA |

What is claimed is:

1. A chimeric or recombinant nucleic acid comprising a polynucleotide sequence that has at least about 95% sequence identity to at least one polynucleotide sequence selected from the group of SEQ ID NO:1 to SEQ ID NO:7 or a complementary polynucleotide sequence thereof.

2. The chimeric or recombinant nucleic acid of claim 1, wherein said chimeric or recombinant nucleic acid produces a chimeric or recombinant human immunodeficiency virus type 1 (HIV-1) variant that exhibits enhanced replication in macaque monkey cells compared to the replication of an HIV-1 virus in macaque monkey cells.

3. The chimeric or recombinant nucleic acid of claim 2, wherein a first polynucleotide subsequence comprising an HIV-1 nef gene has been replaced with a second polynucleotide subsequence comprising a simian immunodeficiency virus (SW) nef gene.

4. The chimeric or recombinant nucleic acid of claim 1, wherein the polynucleotide sequence comprises a polynucleotide sequence selected from the group of SEQ ID NOS:1-7 or a complementary polynucleotide sequence thereof.

5. A chimeric or recombinant nucleic acid comprising a polynucleotide sequence selected from the group of:
(a) a polynucleotide sequence that encodes nine polypeptides, said nine polypeptides comprising the amino acid sequences of SEQ ID NO:8 to SEQ ID NO:16, or a complementary polynucleotide sequence thereof;
(b) a polynucleotide sequence that encodes nine polypeptides, said nine polypeptides comprising the amino acid sequences of SEQ ID NO:17, SEQ ID NO:9, SEQ ID NO:18, SEQ ID NO:11, SEQ ID NO:12, SEQ ID NO:13, SEQ ID NO:14, SEQ ID NO:15, and SEQ ID NO:19, or a complementary polynucleotide sequence thereof;
(c) a polynucleotide sequence that encodes nine polypeptides, the nine polypeptides comprising the amino acid sequences of SEQ ID NO:8, SEQ ID NO:20, SEQ ID NO:10, SEQ ID NO:1, SEQ ID NO:12, SEQ ID NO:13, SEQ ID NO:14, SEQ ID NO:14, and SEQ ID NO:21, respectively, or a complementary polynucleotide sequence thereof;
(d) a polynucleotide sequence that encodes nine polypeptides, the nine polypeptides comprising the amino acid sequences of SEQ ID NO:22, SEQ ID NO:23, SEQ ID NO:10, SEQ ID NO:11, SEQ ID NO:12, SEQ ID NO:24, SEQ ID NO:14, SEQ ID NO:25, and SEQ ID NO:26, or a complementary polynucleotide sequence thereof;
(e) a polynucleotide sequence that encodes nine polypeptides, the nine polypeptides comprising the amino acid sequences of SEQ ID NO:27, SEQ ID NO:9, SEQ ID NO:28, SEQ ID NO:11, SEQ ID NO:12, SEQ ID NO:13, SEQ ID NO:14, SEQ ID NO:29, and SEQ ID NO:30, or a complementary polynucleotide sequence thereof;
(f) a polynucleotide sequence that encodes nine polypeptides, the nine polypeptides comprising the amino acid sequences of SEQ ID NO:8, SEQ ID NO:9, SEQ ID NO:31, SEQ ID NO:11, SEQ ID NO:12, SEQ ID NO:13, SEQ ID NO:14, SEQ ID NO:32, and SEQ ID NO:33, or a complementary polynucleotide sequence thereof; and
(g) a polynucleotide sequence that encodes nine polypeptides, the nine polypeptides comprising the amino acid sequences of SEQ ID NO:34, SEQ ID NO:35, SEQ ID NO:10, SEQ ID NO:36, SEQ ID NO:12, SEQ ID NO:13, SEQ ID NO:37, SEQ ID NO:38, and SEQ ID NO:39, or a complementary polynucleotide sequence thereof.

6. The chimeric or recombinant nucleic acid of claim 1, wherein the polynucleotide sequence comprises a deoxyribonucleic acid (DNA) polynucleotide sequence.

7. A chimeric or recombinant nucleic acid comprising a polynucleotide sequence comprising from at least about nucleic acid residue 530 to at least about nucleic acid residue 9859 of the chimeric or recombinant nucleic acid of claim 1, wherein each thymine residue is replaced by a uracil residue, or a complementary polynucleotide sequence thereof.

8. A chimeric or recombinant nucleic acid comprising a polynucleotide sequence of the chimeric or recombinant nucleic acid of claim 1, wherein each thymine is replaced by a uracil, or a complementary polynucleotide sequence thereof.

9. A chimeric or recombinant RNA polynucleotide sequence comprising from at least about nucleic acid residue 530 to at least about nucleic acid residue 9859 of a polynucleotide sequence selected from the group of SEQ ID NO:1 to SEQ ID NO:7, wherein each thymine is replaced by a uracil, or a complementary polynucleotide sequence thereof.

10. A chimeric or recombinant nucleic acid comprising a polynucleotide sequence comprising a polynucleotide sequence selected from the group of SEQ ID NO:1 to SEQ ID NO:7, wherein each thymine is replaced by a uracil, or a complementary sequence of said polynucleotide sequence.

11. A chimeric or recombinant ribonucleic acid (RNA) comprising an RNA polynucleotide sequence transcribed from at least about nucleic acid residue 530 to at least about nucleic acid residue 9859 of a nucleic acid comprising a polynucleotide sequence selected from the group of SEQ ID NO:1 to SEQ ID NO:7, or a complementary sequence of said RNA polynucleotide sequence.

12. A chimeric or recombinant nucleic acid comprising a polynucleotide sequence comprising from at least about nucleic acid residue 863 to at least about nucleic acid residue 4305 of the chimeric or recombinant nucleic acid of claim 1.

13. The chimeric or recombinant nucleic acid of claim 12, comprising a polynucleotide sequence comprising from at least about nucleic acid residue 785 to at least about nucleic acid residue 4305 of the chimeric or recombinant nucleic acid of claim 1.

14. A chimeric or recombinant nucleic acid comprising a nucleotide sequence of an HIV-1 virus in which a nucleotide subsequence corresponding to a gag coding region, a pro coding region, and a reverse transcriptase (RT) coding region of the HIV-1 nucleotide sequence is replaced by the chimeric or recombinant nucleic acid of claim 12.

15. The chimeric or recombinant nucleic acid of claim 14, wherein the HIV-1 virus comprises DH12.

16. A chimeric or recombinant nucleic acid comprising a polynucleotide sequence comprising from at least about nucleic acid residue 863 to at least about nucleic acid residue 2365 of the chimeric or recombinant nucleic acid of claim 1.

17. A chimeric or recombinant nucleic acid comprising a nucleotide sequence of an HIV-1 virus in which a nucleotide subsequence corresponding to the gag coding region gene of the HIV-1 virus nucleotide sequence is replaced by the chimeric or recombinant nucleic acid of claim 16.

18. The chimeric or recombinant nucleic acid of claim 17, wherein the HIV-1 virus comprises DH12.

19. A chimeric or recombinant nucleic acid comprising a polynucleotide sequence comprising from at least about nucleic acid residue 2158 to at least about nucleic acid residue 4305 of the chimeric or recombinant nucleic acid of claim 1.

20. A chimeric or recombinant nucleic acid comprising an HIV-1 virus polynucleotide sequence in which the nucleotide subsequence corresponding to a protease and reverse transcriptase coding regions of the HIV-1 virus nucleotide sequence is replaced by the chimeric or recombinant nucleic acid of claim 19.

21. The chimeric or recombinant nucleic acid of claim 20, wherein the HIV-1 virus comprises DH12.

22. A vector comprising the chimeric or recombinant nucleic acid of claim 1.

23. The vector of claim 22, wherein the vector comprises a plasmid, a cosmid, or a phage, or encodes a virus or virus-like particle (VLP).

24. The vector of claim 22, wherein the vector comprises an expression vector.

25. A cell comprising the vector of claim 22.

26. A cell comprising at least one chimeric or recombinant nucleic acid of claim 1.

27. The cell of claim 26, wherein the cell expresses at least one polypeptide encoded by the at least one nucleic acid.

28. A composition comprising at least one nucleic acid of claim 1 and an excipient.

29. The composition of claim 28, wherein the excipient is a pharmaceutically acceptable excipient.

30. A modified, recombinant, or chimeric HIV-1 virus comprising at least one chimeric or recombinant nucleic acid of claim 1.

31. A modified, recombinant, or chimeric HIV-1 virus produced by expression or translation of at least one chimeric or recombinant nucleic acid of claim 1 in a population of primate cells.

32. A modified, recombinant, or chimeric HIV-1 virus produced by expression or translation of an RNA nucleic acid in a population of primate cells, said RNA nucleic acid comprising from at least about nucleic acid residue 530 to at least about nucleic acid residue 9859 of a chimeric or recombinant nucleic acid of claim 1, wherein each thymine is replaced by a uracil, or a complementary sequence thereof.

33. A modified, recombinant, or chimeric HIV-1 virus comprising an RNA polynucleotide sequence, said RNA polynucleotide sequence comprising from at least about nucleic acid residue 530 to at least about nucleic acid residue 9859 of a chimeric or recombinant nucleic acid of claim 1, wherein each thymine is replaced by a uracil, or a complementary sequence thereof.

34. A modified, recombinant, or chimeric HIV-1 virus comprising an RNA polynucleotide sequence, said RNA polynucleotide sequence comprising from at least about nucleic acid residue 530 to at least about nucleic acid residue 9859 of a chimeric or recombinant nucleic acid of SEQ ID NO:1 to SEQ ID NO:7, wherein each thymine is replaced by a uracil, or a complementary sequence thereof.

35. The modified, recombinant, or chimeric HIV-1 virus of claim 33, wherein said virus replicates in macaque monkey cells in vivo or in vitro.

36. The modified, recombinant, or chimeric HIV-1 virus of claim 35, wherein the macaque monkey cells comprise pig-tailed macaque monkey cells.

37. The modified, recombinant, or chimeric HIV-1 virus of claim 36, wherein the modified, recombinant, or chimeric HIV-1 virus exhibits enhanced replication in a population of macaque monkey cells compared to replication of an HIV-1 virus in a population of macaque monkey cells.

38. The modified, recombinant, or chimeric HIV-1 virus of claim 37, wherein the modified, recombinant, or chimeric HIV-1 virus grows to a higher titer in a population of macaque monkey cells than does the HIV-1 virus in a population of macaque monkey cells.

39. The modified, recombinant, or chimeric HIV-1 virus of claim 37, wherein the modified, recombinant, or chimeric HIV-1 virus replicates for a longer period of time in a population of macaque monkey cells than does the HIV-1 virus in a population of macaque monkey cells.

40. The modified, recombinant, or chimeric HIV-1 virus of claim 37, wherein the modified, recombinant, or chimeric HIV-1 virus grows at a faster rate in a population of macaque monkey cells than does the HIV-1 virus in a population of macaque monkey cells.

41. The modified, recombinant, or chimeric HIV-1 virus of claim 33, wherein the modified, recombinant, or chimeric HIV-1 virus exhibits replication in vivo in a macaque monkey.

42. The modified, recombinant, or chimeric HIV-1 virus of claim 34, wherein the modified, recombinant, or chimeric HIV-1 virus exhibits replication in vivo in a macaque monkey.

43. The modified, recombinant, or chimeric HIV-1 virus of claim 42, wherein the macaque monkey comprises a pig-tailed macaque monkey.

44. The modified, recombinant, or chimeric HIV-1 virus of claim 42, wherein the modified, recombinant, or chimeric HIV-1 virus exhibits enhanced replication in vivo in the pig-tailed macaque monkey compared to replication of an HIV-1 virus in vivo in said pig-tailed macaque monkey.

45. A cell comprising the modified, recombinant, or chimeric HIV-1 virus of claim 33.

46. The cell of claim 45, wherein the cell comprises a primate cell.

47. The cell of claim 46, wherein the primate cell comprises a human cell.

48. The cell of claim 46, wherein the primate cell comprises a macaque monkey cell.

49. A composition comprising the modified, recombinant, or chimeric HIV-1 virus of claim 33 or claim 34 and an excipient.

50. The composition of claim 49, wherein the excipient is a pharmaceutically acceptable excipient.

51. A non-human primate comprising or infected with the modified, recombinant, or chimeric HIV-1 virus of claim 33.

52. The non-human primate of claim 51, wherein the non-human primate is a macaque monkey.

53. A non-human primate comprising at least one nucleic acid of claim 1.

54. The non-human primate of claim 53, wherein the non-human primate is a macaque monkey.

55. The non-human primate of claim 51, wherein the macaque monkey is a pig-tailed macaque monkey.

56. The non-human primate of claim 52, wherein the modified, recombinant, or chimeric HIV-1 virus replicates for a longer period of time in the macaque monkey than does an HIV-1 virus in the macaque monkey.

57. The non-human primate of claim 52, wherein the macaque monkey comprising the modified, recombinant, or chimeric HIV-1 virus exhibits a decrease in a population of CD4+ T cells.

58. The non-human primate of claim 52, wherein the macaque monkey comprising the modified, recombinant, or chimeric HIV-1 virus exhibits an increase in viremia.

59. The non-human primate of claim 52, wherein the macaque monkey comprising the modified, recombinant, or chimeric HIV-1 virus exhibits at least one symptom of HIV-1 infection that is sustained for a longer period of time than the period of time the symptom of HIV-1 infection lasts in a macaque monkey comprising an HIV-1 virus.

60. The non-human primate of claim 52, wherein the macaque monkey comprising the modified, recombinant, or chimeric HIV-1 virus exhibits at least one symptom of HIV infection.

61. The non-human primate of claim 52, wherein the macaque monkey comprising the modified, recombinant, or chimeric HIV-1 virus exhibits at least one symptom associated with acquired immunodeficiency disease syndrome (AIDS).

62. A method for producing a non-human mammalian cell comprising a modified or chimeric HIV-1 virus that exhibits enhanced replication in a non-human mammalian cell compared to replication of an HIV-1 virus in said non-human mammalian cell, said method comprising administering to the non-human mammalian cell in vitro or in vivo a modified or chimeric HIV-1 virus comprising the nucleic acid of claim 1.

63. The non-human mammalian cell produced by the method of claim 62.

64. The non-human mammalian cell of claim 63, wherein the mammalian cell is a macaque monkey cell.

65. A method for producing a non-human mammalian cell comprising a modified or chimeric HIV-1 virus that exhibits enhanced replication in a non-human mammalian cell compared to replication of an HIV-1 virus in said non-human mammalian cell, said method comprising administering to the non-human mammalian cell the modified, recombinant, or chimeric HIV-1 virus of claim 33.

66. A method for producing a macaque monkey comprising a modified or chimeric HIV-1 virus that exhibits enhanced replication in a macaque monkey cell compared to replication of an HIV-1 virus in said macaque monkey cell, said method comprising administering to a population of cells of the macaque monkey a modified, recombinant, or chimeric HIV-1 virus comprising the nucleic acid of claim 1 in an amount sufficient to cause an HIV infection in said population of cells of the macaque monkey.

67. The method of claim 66, wherein at least one symptom of HIV infection is produced in the macaque monkey or in a population of cells of the macaque monkey.

68. A macaque monkey produced by the method of claim 67.

69. A method for producing a macaque monkey comprising or infected with a modified, recombinant, or chimeric HIV-1 virus that exhibits enhanced replication in a macaque monkey cell compared to replication of an HIV-1 virus in said macaque monkey cell, said method comprising administering to a population of cells of the macaque monkey the modified, recombinant, or chimeric HIV-1 virus of claim 33 in an amount sufficient to cause an HIV infection in said population of cells of the macaque monkey.

70. A chimeric or recombinant polypeptide comprising an amino acid sequence selected from the group of:
(a) an amino acid sequence that has at least about 95% sequence identity to at least one sequence from the group of SEQ ID NOS:8, 17, 22, 27, and 34;
(b) an amino acid sequence that has at least about 95% sequence identity to at least one amino acid sequence of the group of SEQ ID NOS:9, 20, 23, and 35;
(c) SEQ ID NOS:10, 18, 28, and 31;
(d) SEQ ID NOS:11 and 36;
(e) SEQ ID NO:12;
(f) SEQ ID NOS:13 and 24;
(g) SEQ ID NOS:14 and 37;
(h) SEQ ID NOS:15, 25, 29, 32, and 38; and
(i) SEQ ID NOS:16, 19, 21, 26, 30, 33, and 39.

71. A composition comprising the chimeric or recombinant polypeptide of claim 70 and an excipient.

72. The composition of claim 71, wherein the excipient is a pharmaceutically acceptable excipient.

73. A cell-culture derived progeny of the modified, recombinant, or chimeric HIV-1 virus of claim 33.

74. The cell-culture derived progeny of claim 73, wherein the progeny exhibits replication in a macaque monkey and the macaque monkey comprising the progeny exhibits at least one symptom of HIV infection.

75. An evolved virus produced by passaging a viral isolate at least one time through macaque monkey cells, tissue, or blood, wherein the viral isolate comprises the modified, recombinant, or chimeric virus of claim 33.

76. The evolved virus of claim 75, wherein a macaque monkey comprising said evolved virus develops at least one symptom of HIV infection.

77. The evolved virus of claim 75, wherein said evolved virus exhibits enhanced replication in macaque monkey cells, tissue, or blood compared to replication in macaque monkey cells, tissue, or blood of the modified, recombinant, or chimeric virus prior to a first passage.

78. A method for producing an evolved HIV-1 virus that replicates and causes at least one symptom of HIV infection in a macaque monkey, the method comprising passaging a viral isolate comprising a virus in vivo by one or more successive passages through macaque monkey cells, tissue, or blood, wherein prior to the first passage the virus comprised a modified, recombinant, or chimeric virus of claim 1.

79. A method for producing a macaque monkey exhibiting at least one symptom of HIV infection, said method comprising administering to the macaque monkey a viral isolate comprising a virus that has been passaged in vivo at least one time through macaque monkey cells, tissue or blood prior to administration to the macaque monkey, wherein prior to the first passage the comprised a modified, recombinant, or chimeric nucleic acid of claim 1.

80. A method of producing a further modified or recombinant nucleic acid comprising mutating or recombining the chimeric or recombinant nucleic acid of claim 1.

81. The method of claim 80, comprising recursively recombining the chimeric or recombinant nucleic acid with one or more additional nucleic acids.

82. A further modified or recombinant nucleic acid produced by the method of claim 80, wherein a modified, recombinant, or chimeric HIV-1 virus comprising said further modified or recombinant nucleic acid exhibits enhanced replication in non-human mammalian cells.

83. The further modified or recombinant nucleic acid produced by the method of claim 82, wherein the enhanced replication in non-human mammalian cells comprises an ability to replicate at a greater rate or for a longer period in vitro in macaque monkey cells or in vivo in a macaque monkey compared to an HIV-1 virus ability to replicate in vitro in macaque monkey cells or in vivo in a macaque monkey.

84. The method of claim 80, wherein the mutating or recombining is performed in vitro.

85. The method of claim 80, wherein the mutating or recombining is performed in vivo.

86. The method of claim 80, comprising producing at least one library of further modified or recombinant nucleic acids, said library comprising at least one nucleic acid, wherein a modified, recombinant, or chimeric HIV-1 virus comprising said at least one nucleic acid exhibits enhanced replication or improved tropism in non-human mammalian or non-human primate cells.

87. The library produced by the method of claim 86.

88. A population of cells comprising the library of claim 87.

89. A method of screening for an agent that inhibits HIV infection in a non-human primate, said method comprising:
(a) administering a test agent to a first non-human primate;
(b) administering a modified, recombinant, or chimeric HIV-1 virus comprising the nucleic acid of claim 1 to the first non-human primate in an amount sufficient to cause HIV infection;
(c) administering the modified, recombinant, or chimeric HIV-1 virus comprising the nucleic acid of claim 1 to a second non-human primate in said amount;
(d) monitoring a level of HIV infection and/or an appearance of at least one AIDS-associated symptom in each of the first non-human primate and the second non-human primate, wherein a decrease in the level of HIV infection and/or a delay or absence of the appearance of at least one AIDS-associated symptom in the first non-human primate as compared to the level of HIV infection and/or the appearance of at least one AIDS-associated symptom in the second non-human primate indicates that the test agent inhibits HIV infection.

90. The method of claim 89, wherein the first and second non-human primates are macaque monkeys.

91. The method of claim 90, wherein the first and second non-human primates are pig-tailed macaque monkeys.

92. A method for screening for an agent that treats HIV infection said method comprising:
(a) providing a first macaque monkey and a second macaque monkey, each of which comprises the macaque monkey produced by the method of claim 66;
(b) administering a test agent to the first macaque monkey;
(c) monitoring a level of HIV infection and/or an appearance of at least one AIDS-associated symptom in each of the first and second macaque monkeys, wherein a decrease in the level of HIV infection and/or a delay or absence of the appearance of at least one AIDS-associated symptom in the first macaque monkey as compared to the level of HIV infection and/or the appearance of at least one AIDS-associated symptom in the second macaque monkey indicates that the test agent treats HIV infection.

93. A method of screening for an agent that inhibits HIV infection, said method comprising:
(a) administering a test agent to a first population of primate cells;
(b) administering a modified, recombinant, or chimeric HIV-1 virus comprising the nucleic acid of claim 1 to the first population of primate cells in an amount sufficient to cause HIV infection;
(c) administering the modified, recombinant, or chimeric HIV-1 virus comprising the nucleic acid of claim 1 to a second population of primate cells in the amount;
(d) monitoring a level of HIV infection in each of the first population of primate cells and the second population of primate cells, wherein a decrease in the level of HIV infection in the first population of primate cells as compared to the level of HIV infection in the second population of primate cells indicates that the test agent inhibits HIV infection.

94. The method of claim 93, wherein the primate cells are human cells.

95. The method of claim 93, wherein the primate cells are macaque monkey cells.

96. The method of claim 95, wherein the macaque monkey cells are pig-tailed macaque monkey cells.

97. The method of claim 95, wherein the method is performed in vitro.

98. The method of claim 95, wherein the method is performed ex vivo.

99. A method for screening for an agent that treats HIV infection, said method comprising:
(a) providing a first population of macaque monkey cells and a second population of macaque monkey cells, said first and second population of cells comprising one or more cells of claim 48 or one or more cells comprising the cell-culture derived progeny of claim 73;
(b) administering a test agent to the first population of macaque monkey cells;
(c) monitoring a level of HIV infection in the first and second populations of macaque monkey cells, wherein a decrease in the level of HIV infection in the first population of macaque monkey cells as compared to the level of HIV infection in the second population of macaque monkey cells indicates that the test agent inhibits HIV infection.

100. The method of claim 99, the first and second populations of macaque monkey cells are pig-tailed macaque monkey cells.

101. A chimeric nucleic acid that encodes a chimeric or modified HIV-1 virus that exhibits replication in macaque monkey cells, wherein said nucleic acid comprises a polynucleotide sequence of an HIV-1 virus in which a first nucleotide subsequence of the HIV-1 polynucleotide sequence, said nucleotide subsequence comprising a gag coding sequence, a pro coding sequence, and a reverse transcriptase sequence, is substituted with a first chimeric nucleotide subsequence of the nucleic acid sequence of any of SEQ ID NOS:1-7, said first chimeric nucleotide subsequence comprising a chimeric gag coding sequence, chimeric a pro coding sequence, and chimeric reverse transcriptase sequence.

102. The chimeric nucleic acid of claim 101, wherein the first chimeric nucleotide subsequence comprises from at least about nucleic acid residue 785 to at least about nucleic acid residue 4305 of a nucleic acid sequence selected from the group of SEQ ID NOS:1-7.

103. The chimeric nucleic acid of claim 102, wherein the first chimeric nucleotide subsequence comprises from at least about nucleic acid residue 785 to at least about nucleic acid residue 4305 of the nucleic acid sequence of SEQ ID NO:1.

104. The chimeric nucleic acid of claim 101, wherein the HIV-1 virus comprises DH12.

105. The chimeric nucleic acid of claim 101, wherein a second nucleotide subsequence of the HIV-1 polynucleotide sequence corresponding to an HIV-1 nef coding region is substituted with a nucleotide subsequence corresponding to a modified simian immunodeficiency virus (SIV) nef region or gene, said modified SIV nef region or gene comprising a SIV nef polynucleotide sequence in which a first codon encoding an arginine (R) amino acid residue at position 17 of the SIV nef amino acid sequence is substituted with a second codon encoding a tyrosine (Y) amino acid residue and a third codon encoding a glutamine (Q) amino acid residue at position 18 of the SIV nef amino acid sequence has been replaced with a fourth codon encoding a glutamic acid (E) amino acid residue.

106. The chimeric nucleic acid of claim 101, wherein the modified or chimeric virus exhibits enhanced replication in macaque monkey cells.

107. The chimeric or recombinant nucleic acid of claim 106, wherein the macaque monkey cells are pig-tailed macaque monkey cells.

108. The chimeric or recombinant nucleic acid of claim 105, wherein each thymine is substituted with a uracil.

109. A polypeptide encoded by the chimeric or recombinant nucleic acid of claim 101.

110. A modified or chimeric HIV-1 virus comprising the chimeric nucleic acid of claim 101.

111. A chimeric or recombinant nucleic acid that encodes a modified or chimeric HIV-1 virus that exhibits replication in macaque monkey cells, said nucleic acid comprising a polynucleotide sequence of an HIV-1 virus in which a nucleotide subsequence of the HIV-1 virus polynucleotide sequence, said nucleotide subsequence comprising the int, vif, vpr, rev, tat, vpu, and env coding regions is substituted with a chimeric or recombinant nucleotide subsequence comprising a chimeric or recombinant int, vif, vpr, rev, tat, vpu, and env coding regions of a nucleic acid sequence selected from the group of SEQ ID NOS:1-7.

112. The chimeric or recombinant nucleic acid of claim 111, wherein the chimeric or recombinant nucleotide subsequence comprising the chimeric or recombinant int, vif, vpr, rev, tat, vpu, and env coding regions comprises from at least about nucleic acid residue 4306 to at least about nucleic acid residue 8547 of a nucleic acid sequence selected from the group of SEQ ID NOS:1-7.

113. The chimeric or recombinant nucleic acid of claim 112, wherein the chimeric or recombinant nucleotide subsequence comprising the chimeric or recombinant int, vif, vpr, rev, tat, vpu, and env coding regions comprises from at least about nucleic acid residue 4306 to at least about nucleic acid residue 8547 of SEQ ID NO:1.

114. The chimeric or recombinant nucleic acid of claim 111, wherein the HIV-1 virus comprises DH12.

115. The chimeric or recombinant nucleic acid of claim 111, wherein a nucleotide subsequence corresponding to an HIV-1 nef coding region is substituted with a nucleotide subsequence corresponding to a modified SIV nef coding region, said modified SIV nef coding region comprising a SIV nef polynucleotide sequence in which a codon encoding an arginine (R) amino acid residue at position 17 of the encoded SIV nef amino acid sequence is substituted with a codon encoding a tyrosine (Y) amino acid residue and a codon encoding a glutamine (Q) amino acid residue at position 18 of the encoded SIV nef amino acid sequence is substituted with a codon encoding a glutamic acid (E) amino acid residue.

116. The chimeric or recombinant nucleic acid of claim 111, wherein the modified or chimeric HIV-1 virus exhibits enhanced replication in macaque monkey cells in vivo.

117. The chimeric or recombinant nucleic acid of claim 116, wherein the macaque monkey cells are pig-tailed macaque monkey cells.

118. The chimeric or recombinant nucleic acid of claim 111, wherein each thymine is substituted with a uracil.

119. A polypeptide encoded by the chimeric or recombinant nucleic acid of claim 118.

120. A modified, recombinant, or chimeric HIV-1 virus comprising the chimeric or recombinant nucleic acid of claim 111.

121. A chimeric or recombinant nucleic acid that encodes a recombinant or chimeric HIV-1 that exhibits replication in macaque monkey cells, said nucleic acid comprising a polynucleotide sequence of an HIV-1 virus in which a nucleotide subsequence of the polynucleotide sequence that comprises a nef-LTR (long-term repeat) coding region is substituted with a chimeric or recombinant nucleotide subsequence comprising a chimeric or recombinant nef-LTR coding region of a nucleic acid selected from the group of SEQ ID NOS:17.

122. The chimeric or recombinant nucleic acid of claim 121, wherein the chimeric or recombinant nucleotide subsequence comprising the chimeric or recombinant nef-LTR coding region comprises from at least about nucleic acid residue 8548 to at least about nucleic acid residue 9942 of a polynucleotide sequence selected from the group of SEQ ID NOS:1-7.

123. The chimeric or recombinant nucleic acid of claim 122, wherein the chimeric or recombinant nucleotide subsequence comprising the chimeric or recombinant nef-LTR coding region comprises from at least about nucleic acid residue 8548 to at least about nucleic acid residue 9942 of SEQ ID NO:1.

124. The chimeric or recombinant nucleic acid of claim 121, wherein the HIV-1 virus comprises DH12.

125. The chimeric or recombinant nucleic acid of claim 121, wherein a nucleotide subsequence corresponding to an HIV-1 nef coding region is substituted with a nucleotide sequence corresponding to a modified SIV nef coding region, said modified SIV nef coding region comprising a SIV nef nucleotide sequence in which a codon encoding an arginine (R) amino acid residue at position 17 of the SIV nef amino acid sequence is substituted with a codon encoding a tyrosine (Y) amino acid residue, and a codon encoding a glutamine (Q) amino acid residue at position 18 of the SIV nef amino acid sequence is substituted with a codon encoding a glutamic acid (E) amino acid residue.

126. The chimeric or recombinant nucleic acid of claim 121, wherein the encoded modified or chimeric HIV-1 virus exhibits enhanced replication in macaque monkey cells in vivo.

127. The chimeric or recombinant nucleic acid of claim 126, wherein the macaque monkey cells are pig-tailed macaque monkey cells.

128. The chimeric or recombinant nucleic acid of claim 121, wherein each thymine is replaced by a uracil in the polynucleotide sequence.

129. A polypeptide encoded by the chimeric or recombinant nucleic acid of claim 128.

130. A modified or chimeric HIV-1 virus comprising the chimeric or recombinant nucleic acid of claim 128.

131. A chimeric or recombinant nucleic acid that encodes a modified or chimeric HIV-1 virus that exhibits replication in macaque monkey cells, said nucleic acid comprising an RNA genome corresponding to a polynucleotide sequence selected from the group of SEQ ID NOS:17 in which each thymine residue is replaced by a uracil residue, wherein a first nucleotide subsequence of the first RNA genome comprising a first gag gene, a first protease gene, and a first reverse transcriptase (RT) gene is replaced by a second nucleotide subsequence comprising a second gag gene, a second protease gene, and a second reverse transcriptase gene of a second RNA genome of an HIV-1 virus.

132. The chimeric or recombinant nucleic acid of claim 131, wherein the first nucleotide subsequence comprising the first gag gene, the first protease gene, and the first reverse transcriptase gene of the first RNA genome comprises from at least about nucleic acid residue 863 to at least about nucleic acid residue 4305 of the polynucleotide sequence selected from the group of SEQ ID NOS:1-7 wherein each thymine is replaced by a uracil.

133. The chimeric or recombinant nucleic acid of claim 132, wherein the first nucleotide subsequence comprising the first gag gene, the first protease gene, and the first reverse transcriptase gene of the first RNA genome comprises from at least about nucleic acid residue 785 to at least about nucleic acid residue 4305 of the polynucleotide sequence selected from the group of SEQ ID NOS:1-7 wherein each thymine residue is replaced by a uracil residue.

134. The chimeric or recombinant nucleic acid of claim 131, wherein the HIV-1 virus comprises DH12.

135. The chimeric or recombinant nucleic acid of claim 131, wherein the HIV-1 virus comprises DH12 and the second nucleotide subsequence comprising the second gag gene, the second pro gene, and the second reverse transcriptase gene of the second RNA genome of DH12 comprises from at least about nucleic acid residue 710 to at least about nucleic acid residue 4223 of the DH12 polynucleotide sequence.

136. A chimeric or recombinant nucleic acid that encodes a modified or chimeric HIV-1 virus that exhibits replication in macaque monkey cells, said nucleic acid comprising a recombinant DNA polynucleotide sequence corresponding to nucleic acid residues 530 to 9859 of a first polynucleotide sequence selected from the group of SEQ ID NOS:1-7, wherein a first nucleotide subsequence of the first DNA polynucleotide sequence comprising a first gag gene, a first protease gene, and a first reverse transcriptase gene is replaced by a second nucleotide subsequence comprising a second gag gene, a second protease gene, and a second reverse transcriptase gene of an HIV-1 virus DNA polynucleotide sequence.

137. The chimeric or recombinant nucleic acid of claim 131, wherein the modified or chimeric HIV-1 virus exhibits enhanced replication in macaque monkey cells in vivo.

138. The chimeric or recombinant nucleic acid of claim 137, wherein the macaque monkey cells are pig-tailed macaque monkey cells.

139. A polypeptide encoded by the chimeric or recombinant nucleic acid of claim 131.

140. A modified or chimeric HIV-1 virus comprising the chimeric or recombinant nucleic acid of claim 131.

141. A chimeric or recombinant nucleic acid that encodes a modified or chimeric HIV-1 virus variant that exhibits replication in macaque monkey cells, said nucleic acid comprising an RNA polynucleotide sequence comprising from about nucleic acid residue 530 to about nucleic acid residue 9859 of a polynucleotide sequence selected from the group of SEQ ID NOS:1-7 in which each thymine is substituted with a uracil, wherein a first nucleotide subsequence of said RNA sequence comprising first integrase (int), vif, vpr, rev, tat, vpu, and envelope (env) coding regions or genes of said RNA sequence is replaced by a second nucleotide subsequence comprising second int, vif, vpr, rev, tat, vpu, and env genes of an HIV-1 virus polynucleotide sequence.

142. The chimeric or recombinant nucleic acid of claim 141, wherein the first RNA sequence comprises from at least about nucleic acid residue 4306 to at least about nucleic acid residue 8547 of a polynucleotide sequence from the group of SEQ ID NOS:1-7 in which each thymine is substituted with a uracil.

143. The chimeric or recombinant nucleic acid of claim 141, wherein the HIV-1 virus comprises DH12.

144. The chimeric or recombinant nucleic acid of claim 141, wherein the HIV-1 virus comprises DH12 and the second polynucleotide subsequence comprising the int, vif, vpr, rev, tat, vpu, and envelope genes of the DH12 polynucleotide sequence comprises from at least about nucleic acid residue 4224 to at least about nucleic acid residue 8465 of the DH12 polynucleotide sequence, wherein each thymine is replaced by a uracil.

145. A chimeric or recombinant nucleic acid that encodes a modified or chimeric HIV-1 virus variant that exhibits replication in macaque monkey cells, said nucleic acid comprising a DNA polynucleotide sequence comprising from about nucleic acid residue 530 to about nucleic acid residue 9859 of a polynucleotide sequence selected from the group of SEQ ID NOS:1-7, wherein a first polynucleotide subsequence of said DNA polynucleotide sequence comprising first int, vif, vpr, rev, tat, vpu, and env genes is replaced by a second polynucleotide subsequence comprising second int, vif, vpr, rev, tat, vpu, and env genes of an HIV-1 virus polynucleotide sequence.

146. The chimeric or recombinant nucleic acid of claim 141 or 145, wherein the modified or chimeric HIV-1 virus exhibits enhanced replication in macaque monkey cells in vivo.

147. The chimeric or recombinant nucleic acid of claim 146, wherein the macaque monkey cells are pig-tailed macaque monkey cells.

148. A polypeptide encoded by the chimeric or recombinant nucleic acid of claim 141 or 145.

149. A modified or chimeric HIV-1 virus comprising the chimeric or recombinant nucleic acid of claim 141 or 145.

150. A chimeric or recombinant nucleic acid that encodes a modified or chimeric HIV-1 virus variant that exhibits replication in macaque monkey cells, said nucleic acid comprising a RNA sequence comprising from about nucleic acid residue 530 to about nucleic acid residue 9859 of a polynucleotide selected from the group of SEQ ID NOS:1-7 wherein each thymine is replaced by a uracil, and wherein a first polynucleotide subsequence of said RNA sequence comprising a first nef-LTR gene is replaced by a second polynucleotide subsequence comprising a second nef-LTR gene of an HIV-1 virus polynucleotide sequence.

151. The nucleic acid of claim 150, wherein the first nef-LTR gene of the RNA sequence comprises from at least about nucleic acid residue 8548 to at least about nucleic acid residue 9942 of the polynucleotide sequence from the group of SEQ ID NOS:1-7, wherein each thymine is replaced by a uracil.

152. The nucleic acid of claim 150, wherein the first nef-LTR gene of the RNA sequence comprises from at least about nucleic acid residue 8548 to at least about nucleic acid residue 9942 of SEQ ID NO:1, wherein each thymine is replaced by a uracil.

153. The nucleic acid of claim 150, wherein the HIV-1 virus comprises DH12.

154. The chimeric or recombinant nucleic acid of claim 150, wherein the HIV-1 virus comprises DH12 and the second polynucleotide subsequence comprising the second nef-LTR gene of the DH12 polynucleotide sequence comprises from at least about nucleic acid residue 8466 to at least about nucleic acid residue 9704 of the DH12 polynucleotide sequence.

155. A chimeric or recombinant nucleic acid that encodes a modified or chimeric virus that exhibits replication in macaque monkey cells, said nucleic acid comprising a DNA polynucleotide sequence comprising from about nucleic acid residue 530 to about nucleic acid residue 9859 of a polynucleotide sequence selected from the group of SEQ ID NOS:1-7, wherein a first polynucleotide subsequence of said DNA sequence comprising a first nef-LTR gene is replaced by a second polynucleotide subsequence comprising a second nef-LTR gene of an HIV-1 virus polynucleotide sequence.

156. The chimeric or recombinant nucleic acid of claim 150 or 155, wherein a third polynucleotide subsequence corresponding to an HIV-1 nef gene is replaced with a fourth polynucleotide subsequence corresponding to a modified SIV nef gene, said modified SIV nef gene comprising a SIV nef polynucleotide sequence in which a first codon encoding an arginine (R) amino acid residue at position 17 of the SIV nef amino acid sequence is replaced with a second codon encoding a tyrosine (Y) amino acid residue and a third codon encoding a glutamine (Q) amino acid residue at position 18 of the SIV nef amino acid sequence has been replaced with a fourth codon encoding a glutamic acid (E) amino acid residue.

157. The chimeric or recombinant nucleic acid of claim 150 or 155, wherein the modified or chimeric HIV-1 virus exhibits enhanced replication in macaque monkey cells in vivo.

158. The chimeric or recombinant nucleic acid of claim 157, wherein the macaque monkey cells are pig-tailed macaque monkey cells.

159. A polypeptide encoded by the chimeric or recombinant nucleic acid of claim 150 or 155.

160. A modified, recombinant, or chimeric HIV-1 virus comprising the chimeric or recombinant nucleic acid of claim 150 or 155.

161. A chimeric or recombinant nucleic acid comprising a polynucleotide sequence that, but for the degeneracy of the genetic code, hybridizes under stringent conditions over substantially the entire length of the chimeric or recombinant nucleic acid of claim 1.

162. A chimeric or recombinant nucleic acid comprising a polynucleotide sequence that, but for the degeneracy of the genetic code, hybridizes under stringent conditions over substantially the entire length of a nucleic acid comprising a polynucleotide sequence selected from the group of SEQ ID NOS:1-7, or a complementary polynucleotide sequence thereof.

163. A chimeric or recombinant polypeptide comprising an amino acid sequence that has at least about 95% amino acid sequence identity to at least one sequence from the group of SEQ ID NOS:8-39, or to a polypeptide fragment thereof, wherein a virus comprising such polypeptide fragment that exhibits enhanced replication in macaque monkey cells compared to the replication of a WT HIV-1 virus in macaque monkey cells

164. A chimeric or recombinant nucleic acid that encodes a polypeptide having at least about 95% amino acid sequence identity to at least one amino acid sequence selected from the group of SEQ ID NOS:8-39, or that encodes a polypeptide fragment thereof, wherein a virus comprising such polypeptide fragment that exhibits enhanced replication in macaque monkey cells compared to the replication of a WT HIV-1 virus in macaque monkey cells.
